(19) United States
(12) Patent Application Publication
(10) Pub. No.: US 2010/0249030 A1
(43) Pub. Date: Sep. 30, 2010

(54) ANTI-MITOTIC AGENT AND AURORA KINASE INHIBITOR COMBINATION AS ANTI-CANCER TREATMENT

(75) Inventor: Andrea Dawn Basso-Porcaro, Guttenberg, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/670,762

(22) PCT Filed: Jul. 28, 2008
(86) PCT No.: PCT/US08/09108

§ 371 Date: Feb. 23, 2010

Related U.S. Application Data
(60) Provisional application No. 60/953,087, filed on Jul. 31, 2007.
(60) Provisional application No. 61/023,985, filed on Jan. 28, 2008.

(51) Int. Cl. A61K 31/4985; A61K 38/38; A61K 31/427; A61K 31/5377; A61P 35/00; A61K 31/55; A61K 31/541; A61K 31/4365; A61K 31/4184; A61K 31/495

(52) U.S. Cl. 514/12; 514/365; 514/249; 514/233.2; 514/217.05; 514/228.5; 514/291; 514/395; 514/254.02; 514/253.1

(57) ABSTRACT

The present invention relates to a method of treating cancer by pretreatment with anti-mitotic agents followed by at least one aurora kinase inhibitor. Extensive illustrations are provided for the antimitotic agents and aurora kinase inhibitors that are useful in the inventive treatment.

### FIELD OF THE INVENTION

The present invention relates to a method of treating cancer by pretreatment with anti-mitotic agents followed by aurora kinase inhibitors. This application claims priority from U.S. provisional patent application Ser. No. 60/953,087 filed Jul. 31, 2007.

### BACKGROUND OF THE INVENTION

Taxanes such as paclitaxel and docetaxel and vinca alkaloids target microtubules, which are responsible for distribution of duplicated sister chromatids to each of the daughter cells. Disruption of microtubules can inhibit cell division and induce apoptosis.

KSP inhibitors interfere with function of mitotic kinesins and therefore disrupt normal mitosis and blocks cell division. Mitotic Kinesin-KSP, known as Eg5, is required for centrosome separation. Cells in which KSP function is inhibited, arrest in mitosis with un-separated centrosomes. (Blangy et al., Cell 1995, 83: 1159-1169, Heald, R., Cell 2000, 102, 399). Mitotic arrest leads to growth inhibition of tumor cells. (Kaiser et al., J. Biol. Chem. 1999, 274: 18925-18931).

Like KSP inhibitor, Cenp-E inhibitors inhibit centrosome separation by inhibiting centrosome associated protein E. resulting in cell cycle arrest with bipolar mitotic spindles and misaligned chromosomes.

Ispinesib and Monastrol are kinesin spindle protein inhibitors. They cause cell cycle arrest by disrupting a kinesin-related motor protein that is necessary for formation of a bipolar spindle. (Mayer et al, Science 1999, Vol. 286, No. 5441, 971-974).

Aurora kinases (Aurora-A, Aurora-B, Aurora-C) are serine/threonine protein kinases that have been implicated in human cancer, such as colon, breast and other solid tumors. In various human cancers over expression of Aurora A and/or Aurora 13 has been observed. In some cases this is a result of gene amplification. Over expression of Aurora kinases correlates with poor survival prognosis.

Aurora kinases are involved in phosphorylation events that regulate the cell cycle. Misregulation of the cell cycle can lead to cellular proliferation and other abnormalities. Aurora A regulates centromere maturation, mitotic entry, bipolar spindle assembly, and chromosome alignment. Aurora B regulates chromatin remodeling, kinetochore-spindle attachment, and cytokinesis. Aurora C expression is limited and its function is thought to be similar to that of Aurora B.

Small molecules that inhibit Aurora kinases have been developed as potential anti-cancer agents. VX-680 (Harrington 2004) and AT9283 are dual Aurora A/B inhibitors. AZD1152 is an Aurora B selective inhibitor (Morlock 2006) and MLN8054 is a reported Aurora A specific inhibitor (Manfredi 2007). Compounds that inhibit Aurora B result in induction of endoreduplication, cells continue through the cell cycle without undergoing cytokinesis and accumulate DNA with >4N content (where 2N DNA represents cells in G1 and 4N represents cell in mitosis).

Reference is also made to R. R. Hoover. and M. W. Harding, Journal of Clinical Oncology, 2007 American Society of Clinical Oncology Annual Meeting Proceedings Part I. Vol. 25, No. 18S (June 20 Supplement), 2007: 14069, which discusses the in vitro use of a reversible kinase inhibitor (MK-0457) that targets Aurora A, B and C in sequential or simultaneous combination with the microtubule targeted agent, Taxotere, on cancer cell lines. It states that synergy occurs with sequential and not simultaneous exposure to both compounds in the short-term viability assays and synergy occurred with simultaneous exposure in the long-term survival assays.

Reference is made to Aurora inhibition in combination with taxanes in Proceedings from the American Association for Cancer Research 2007 Annual Meeting (Apr. 18, 2007). P Phatak et al. (abstract No. 5746) discusses the schedule dependent synergy of a non-specific aurora kinase inhibitor (VE-465), which is similar to MK-0457, which is currently in clinical trials, and paclitaxel in non-small cell lung cancer. It states that VE-465 and paclitaxel exhibit schedule dependent synergy that results in enhanced cell kill with dependence on the cell line. Additionally, Abstract No. 4357 concluded VX-680 and taxotere combination showed enhanced cell growth inhibition and Abstract No. 1819 concluded VE-645 and taxol combination showed enhanced cell growth inhibition. Abstract No. 4359 and No. 3263 concluded AZD1152 in combination with taxol showed enhanced cell growth inhibition.

References have been made to selective Aurora A inhibition and taxanes. Tanaka et al. (Clinical Cancer Research 2007; 13:1331) showed Aurora A siRNA in combination with Taxotere resulted in enhanced apoptosis. Hata et al (Cancer Research 2005; 65: 2899) demonstrated Aurora A siRNA in combination with taxanes showed enhanced cytotoxicity. Ohkubo M et al disclosed in a patent combination of novel aminopyridines having selective Aurora A inhibitory effects with Taxol had enhanced cell growth inhibition.

Yang et al (International Journal of Cancer 2006; 119:2304) found that ectopic expression of Aurora A rendered cells resistant to anti-cancer agents such as Taxol. Similarly, Anand et al (Cancer Cell 2003; 3:51) found elevated Aurora A expression causes resistance to apoptosis induced by Taxol. Anand et al (WO05/002571, filed Jan. 13, 2005) disclosed that over-expression of Aurora A mediates resistance to anti-cancer agents and the resistance can be reduced by inhibiting Aurora kinases or predicted by measuring the expression or activity of Aurora kinases within the cell.

### SUMMARY OF THE INVENTION

This invention provides a pharmaceutical composition for treating or ameliorating cancer comprising at least one first compound, which is an anti-mitotic agent and at least one second compound, which is an aurora kinase inhibitor.

This invention further provides a method of treating or ameliorating cancer comprising administration to a mammal in need of such treatment an amount of at least one first compound, which is an anti-mitotic agent followed by an amount of at least one second compound, which is an aurora kinase inhibitor. The following sections describe the antimitotic agent and the aurora kinase inhibitor in more details.

Non-limiting examples of anti-mitotic agents useful in this invention include:

Taxanes, paclitaxel, docetaxel, Cenp-E Inhibitor (such as for example GSK-923295), Abraxane, Epothilone, Monastrol, and KSP inhibitors. Non-limiting examples of useful KSP inhibitors include Ispinesib SB-715992 (Cytokinetics), as well as the compounds of Formulas A-D shown in paragraphs a-d below:

a. A compound represented by the structural Formula A:

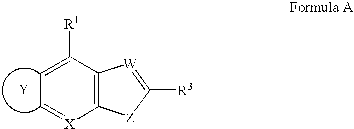

or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²)
X is N or N-oxide;
Z is S, S(═O) or S(═O)₂;
   R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
   each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
   each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
   each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
   or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
   each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
   each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
   each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF₃, —OCF₃, —NR¹⁰C(O)OR¹¹, and —NR¹⁰C(O)R⁴⁰;
   or two R⁸ groups, when attached to the same carbon atom, are optionally taken together with the carbon atom to which they are attached to form a C═O or a C═S group;
   each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
   each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
   each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
   each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
   R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹.
      Such compounds of Formula A in (a) are disclosed in WO 2006/098962, filed Mar. 7, 2006, the content of which is incorporated herein by reference in its entirety;

b. A compound represented by the structural Formula B:

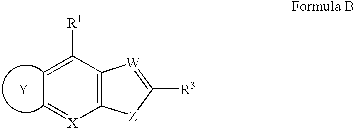

or a pharmaceutically acceptable salt, solvate or ester thereof, wherein:

ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;

each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;

or two R²s on the same carbon atom are optionally taken together with the carbon atom to which they are attached to form a C═O, a C═S or an ethylenedioxy group;

R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)CR⁴⁰R⁴¹)₁₋₅—C(O)—NR⁴R⁵,

### —C(O)N(R⁷)(CR⁴⁰R⁴¹)₁₋₅—OR⁷, —C(S)NR⁷(CH₂)₁₋₅NR⁴R⁵, and

—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;

each of R⁴ and R⁵ is independently selected from the group consisting of H, alkyl, cycloalkyl, cycloalkenyl, heterocyclyl, heterocyclenyl, aryl, heteroaryl,

—OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;

each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;

or two R⁸ groups, when attached to the same carbon, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰) is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,

—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)NR⁴OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

R⁴⁰ and R⁴¹ can be the same or different, each being independently selected from the group consisting of H, alkyl, aryl, heteroaryl, heterocyclyl, heterocyclenyl, cycloalkyl and cycloalkenyl;

each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰,

—CN, —NR¹⁰R¹¹, —C(O)OR¹⁰, —C(O)OR¹⁰, —C(O)NR¹⁰R¹¹, —CF₃, —OCF₃, —N(R¹⁰)C(O)R¹¹, and —NR¹⁰C(O)OR¹¹;

with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹. Such compounds of Formula B in (b) are disclosed in WO2006/098961, filed Mar. 7, 2006, the content of which is incorporated herein by reference in its entirety;

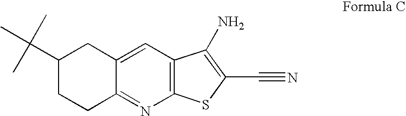

c.

or a pharmaceutically acceptable salt, solvate, or ester thereof.
Such compounds of Formula C in (c) are disclosed in U.S. Pub. No. 2006/0258699, filed Mar. 7, 2006, the content of which is incorporated herein by reference in its entirety; and

d. A compound of Formula D

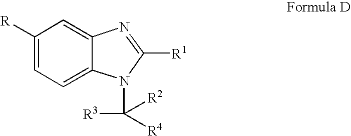

or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl),
   S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl.
   Compounds represented in the present application by Formula (d) are disclosed in PCT US2008/006472, filed May 21, 2008, the content of which is incorporated herein by reference in its entirety;

Non-limiting examples of suitable aurora kinase inhibitors useful in this invention include the compounds represented by Formulas E-K shown below in paragraphs e-k:

e. A compound represented by the structural Formula E:

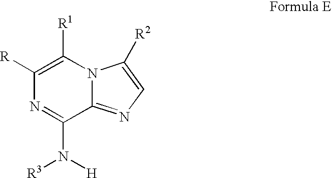

or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
   —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
   —NR⁵R⁶ (wherein the R⁵ and R⁶, together with the N of said
   —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
   —CH₂OR⁵, —S(O)R⁵, —S(O₂)R⁵, —CN, —CHO, —SR⁵, —C(O)OR⁵, —C(O)R⁵, heteroaryl and heterocyclyl;
      R³ is H, alkyl, cycloalkyl, heterocyclyl, aryl or heteroaryl, wherein:
   said alkyl shown above for R³ can be unsubstituted or substituted with one or more moieties which can be the same or different each moiety being independently selected from the group consisting of —OR⁵, alkoxy, heteroaryl, and —NR⁵R⁶;
   said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
      —S(O₂)R⁵; and
   said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
      R⁵ is H, alkyl, aminoalkyl, aryl, heteroaryl, heterocyclyl or cycloalkyl; and
      R⁶ is H, alkyl, aryl, arylalkyl, heteroaryl, heterocyclyl or cycloalkyl;
      further wherein in any —NR⁵R⁶ in Formula I, said R⁵ and R⁶ can optionally be joined together with the N of said —NR⁵R⁶ to form a cyclic ring.
      Such compounds of Formula E in (e) are disclosed in WO2007/058942 filed Nov. 8, 2006, the content of which is incorporated herein by reference in its entirety;

f. A compound represented by the structural formula:

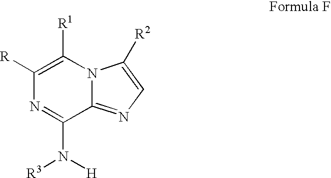

or a pharmaceutically acceptable salt, solvate, ester or prodrug thereof, wherein:

R is selected from the group consisting of H, halogen, aryl, heteroaryl, cycloalkyl, arylalkyl, heterocyclyl, heterocyclylalkyl, alkenyl, alkynyl,

### —C(O)R⁷,

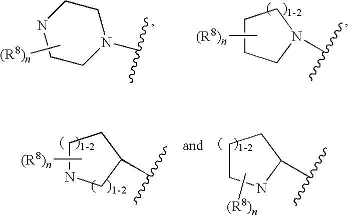

wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷,
—S(O₂)NR⁵R⁶, —N(R⁵)S(O₂)R⁷, —N(R⁵)C(O)R⁷ and —N(R⁵)C(O)NR⁵R⁶;

R¹ is H, halogen or alkyl;

R² is alkyl;

R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₘ—NR⁸, —(CHR⁵)—CH(aryl)₂,

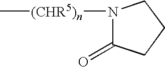

and

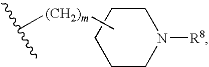

wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and

### —N(R⁵)C(O)NR⁵R⁶;

R⁵ is H or alkyl;

R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and

### —N(R⁵)C(O)NR⁵R⁶;

R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,

—S(O₂)NR⁵R⁶, —N(R⁵)S(O₂)R⁷, —N(R⁵)C(O)R⁷ and —N(R⁵)C(O)NR⁵R⁶;

R⁸ is selected from the group consisting of R⁶, —C(O)NR⁵R⁶,

—S(O₂)NR⁵R⁶, —C(O)R⁷, —C(O₂)R⁶, —S(O₂)R⁷ and —(CH₂)-aryl;

R⁹ is selected from the group consisting of halogen, CN, NR⁵R⁶,

—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;

m is 0 to 4;

n is 1-4; and

p is 0-3.

Such compounds of Formula F in (f) are disclosed in WO2004/026877, filed Sep. 19, 2003, the content of which is incorporated herein by reference in its entirety;

g. A compound selected from the compounds of the formulas:

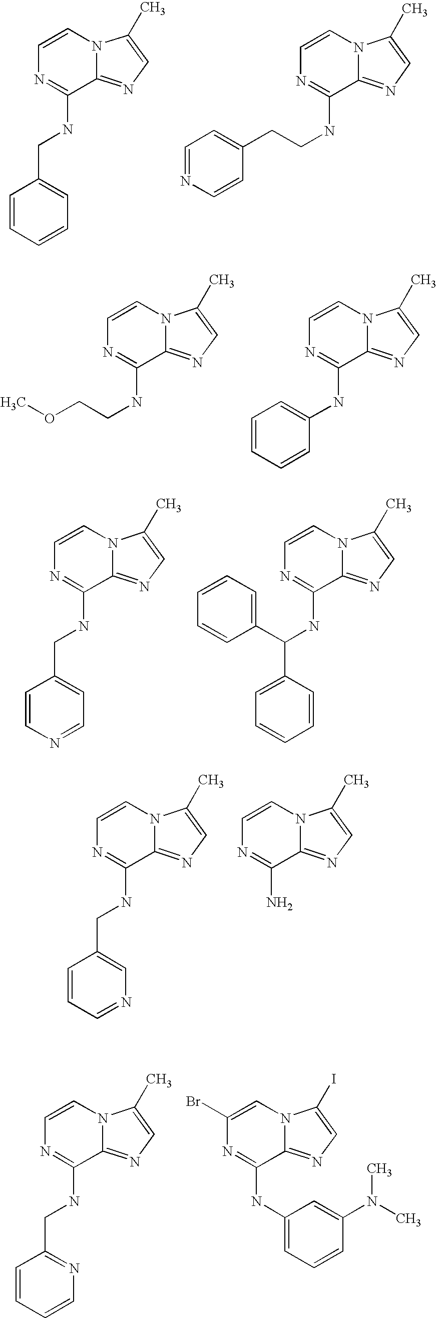

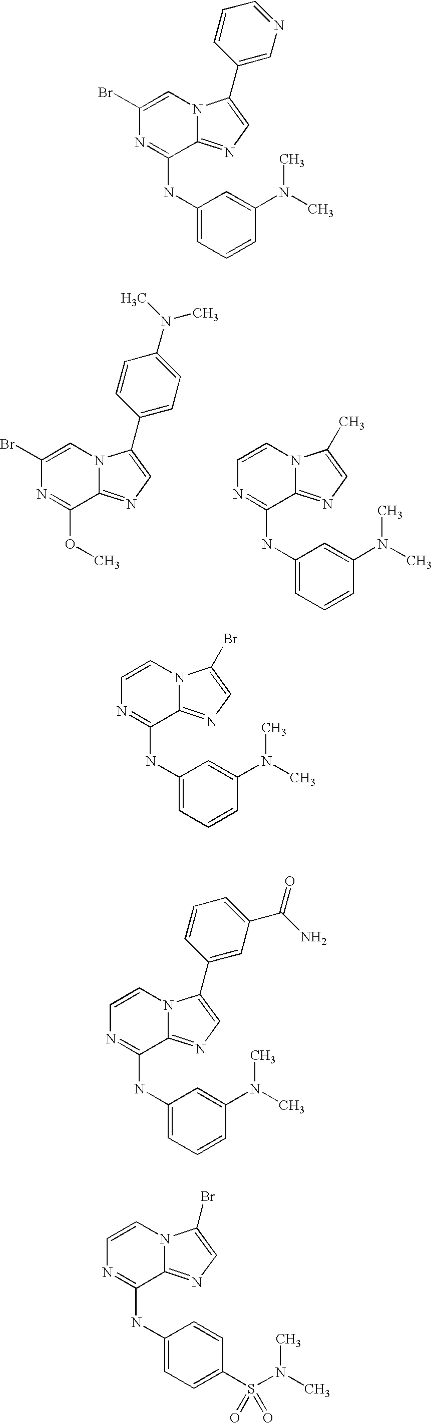

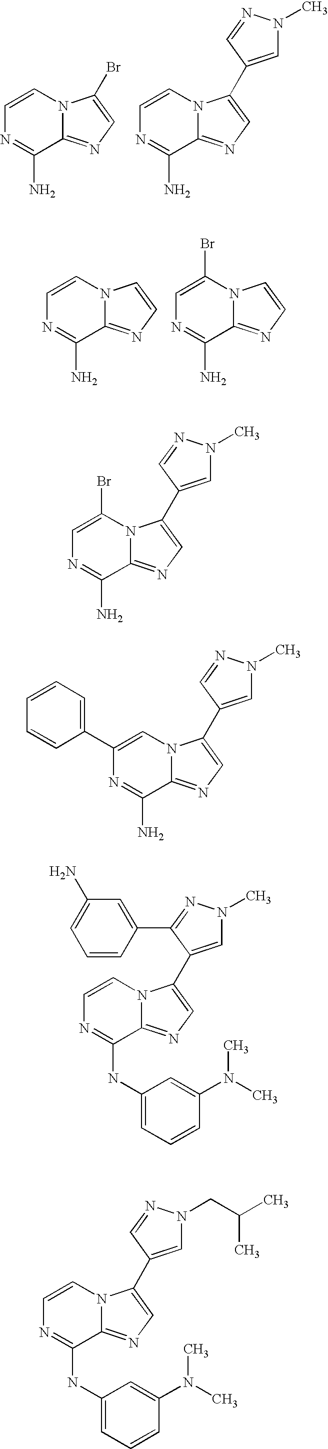

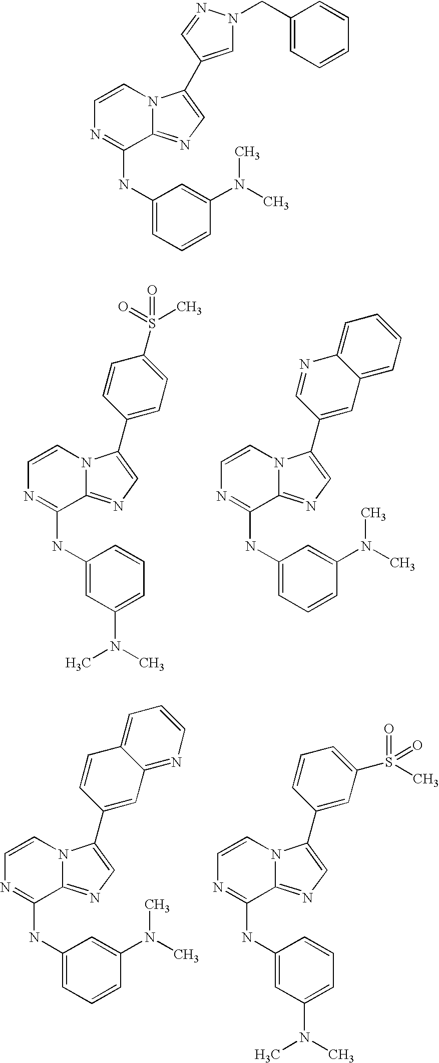

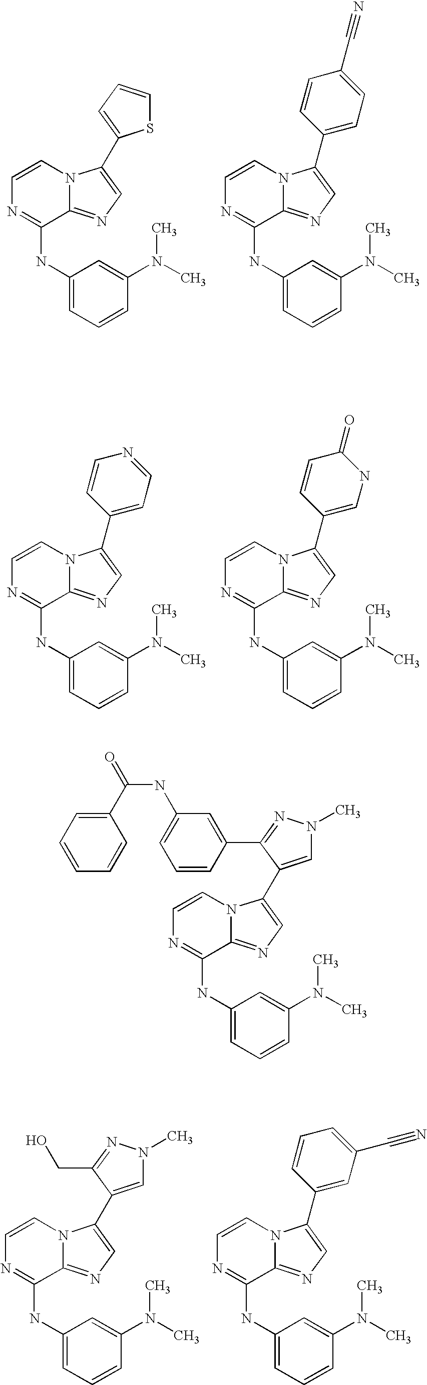

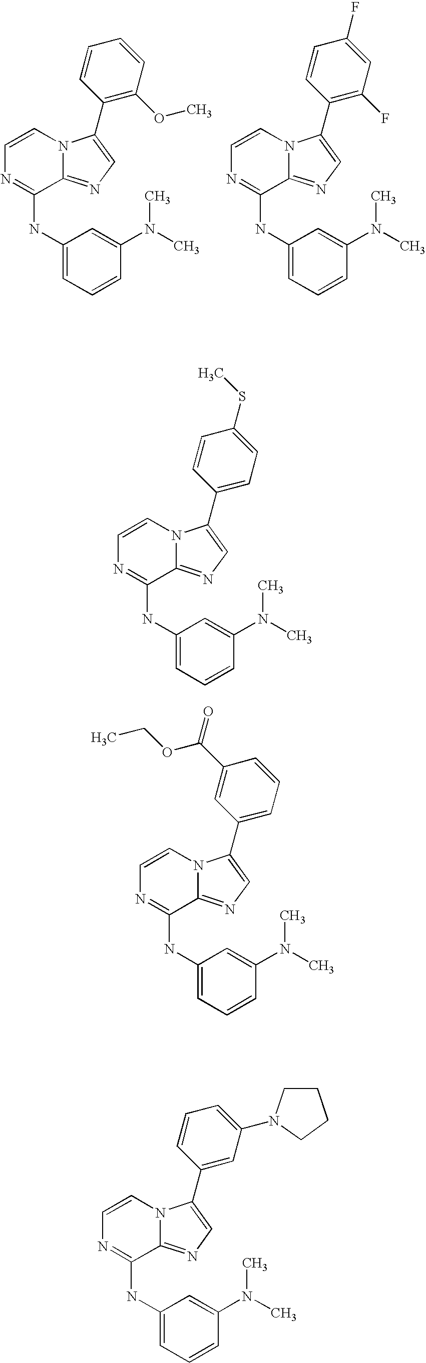

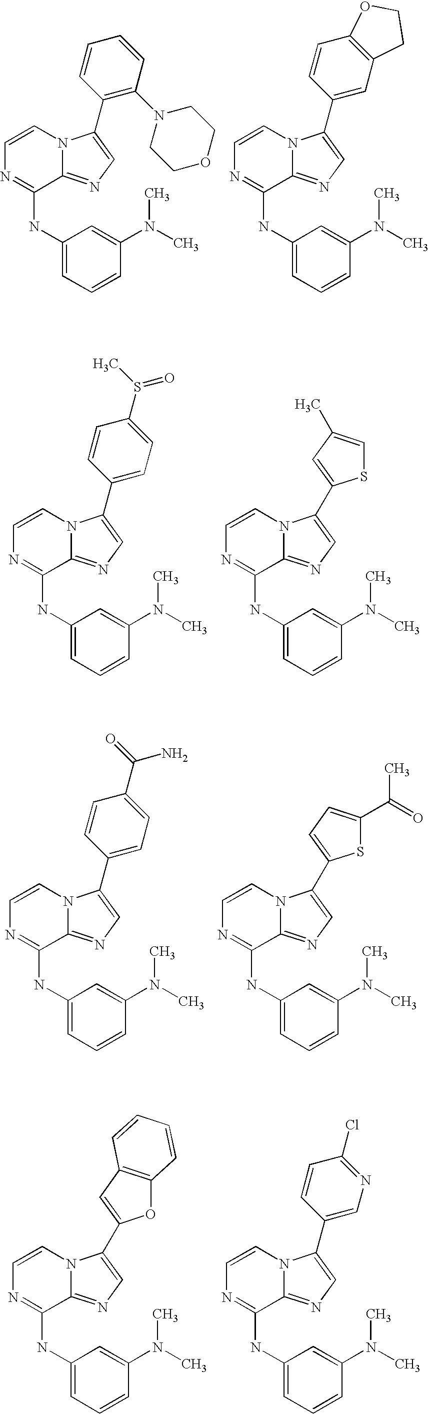

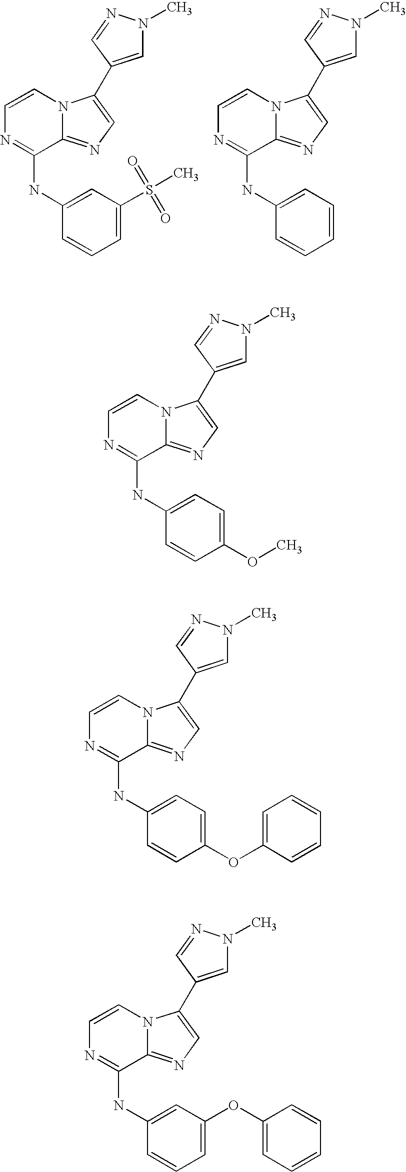

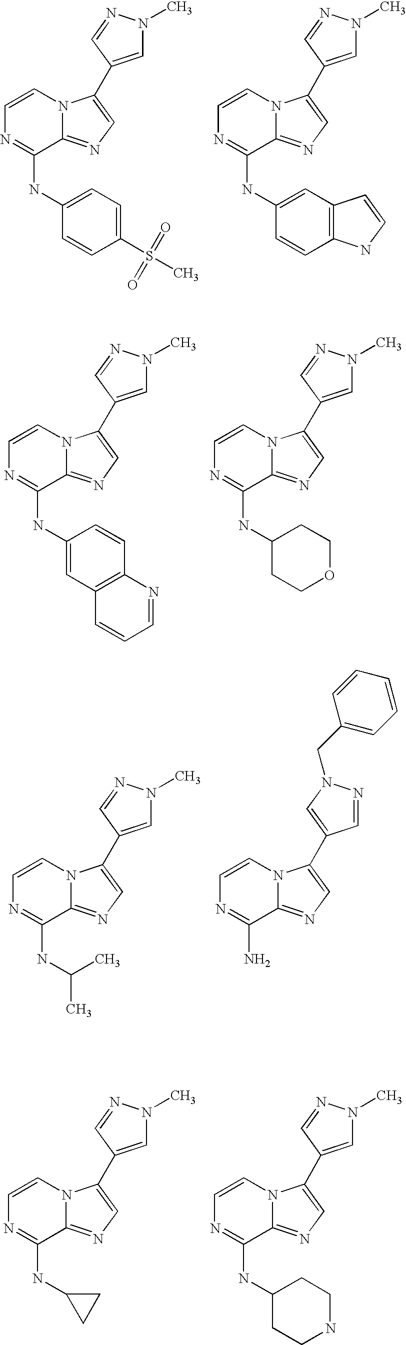

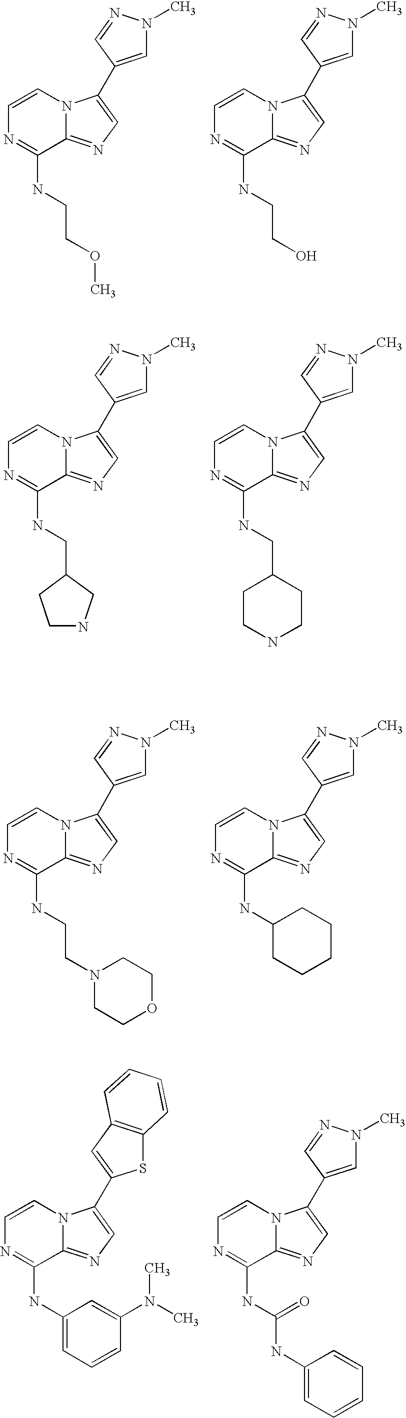

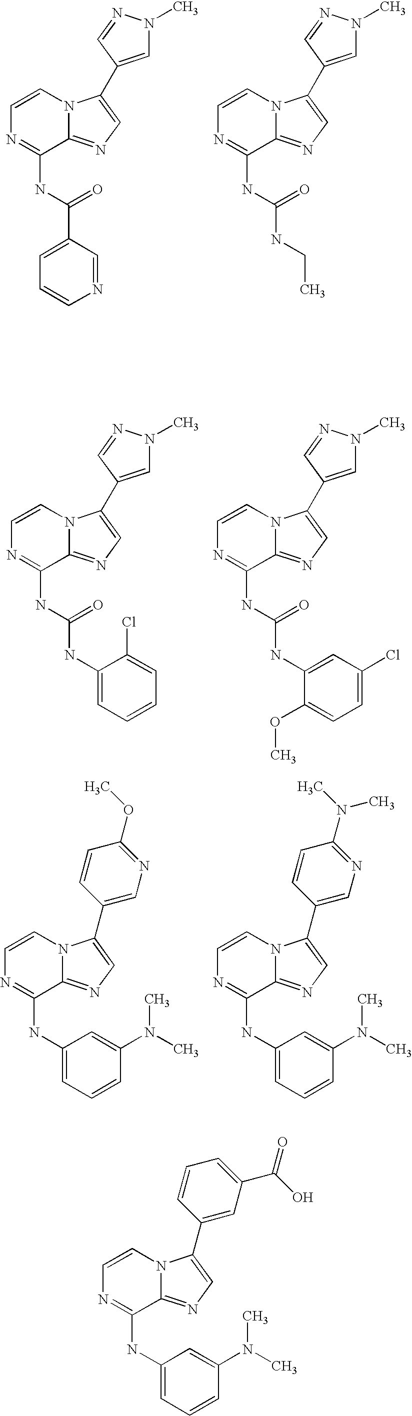

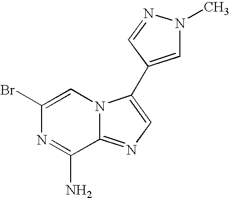

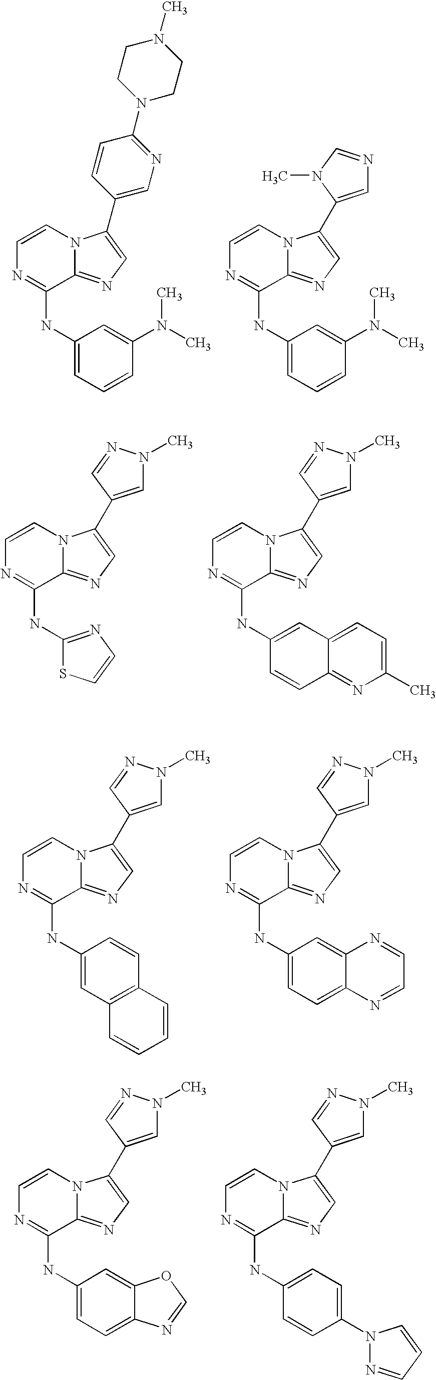

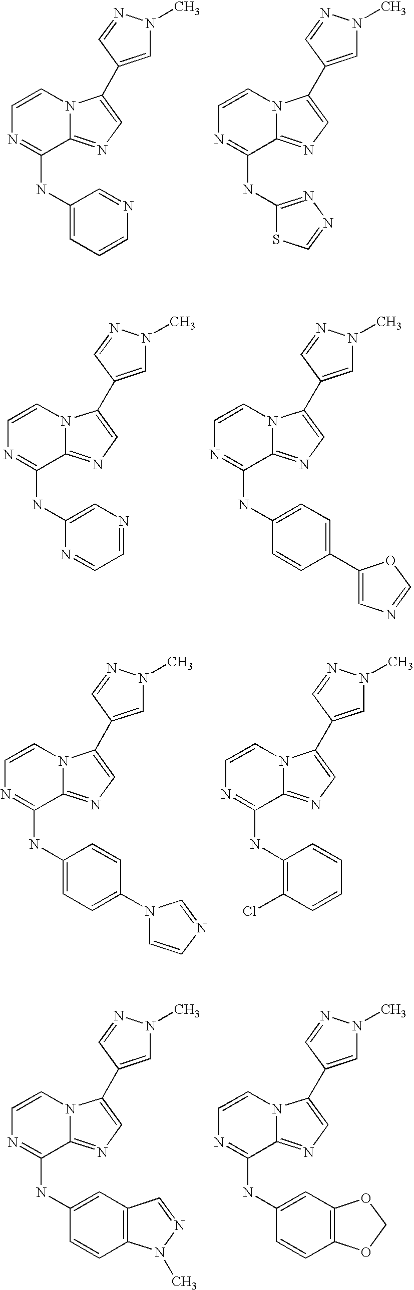

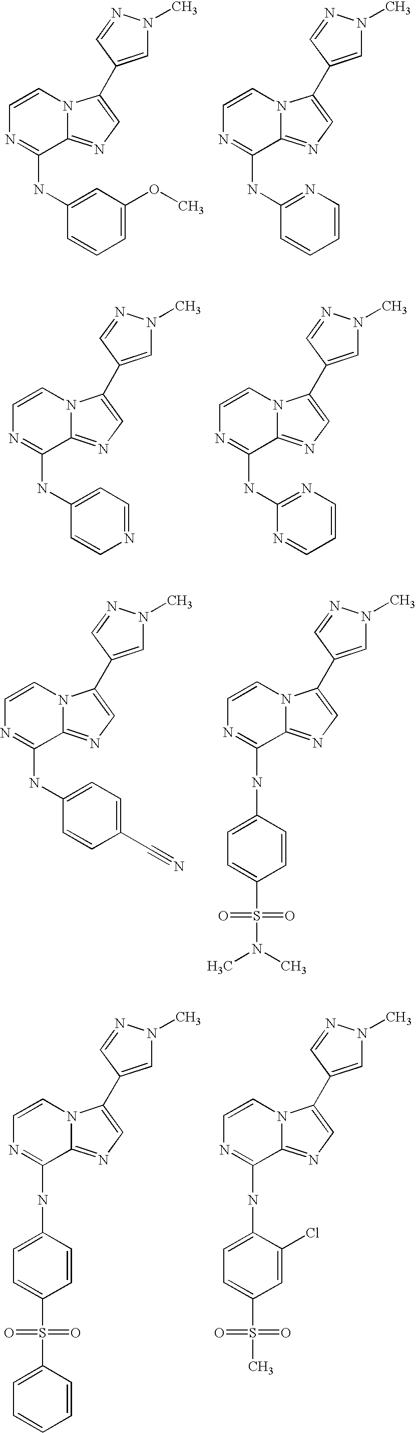

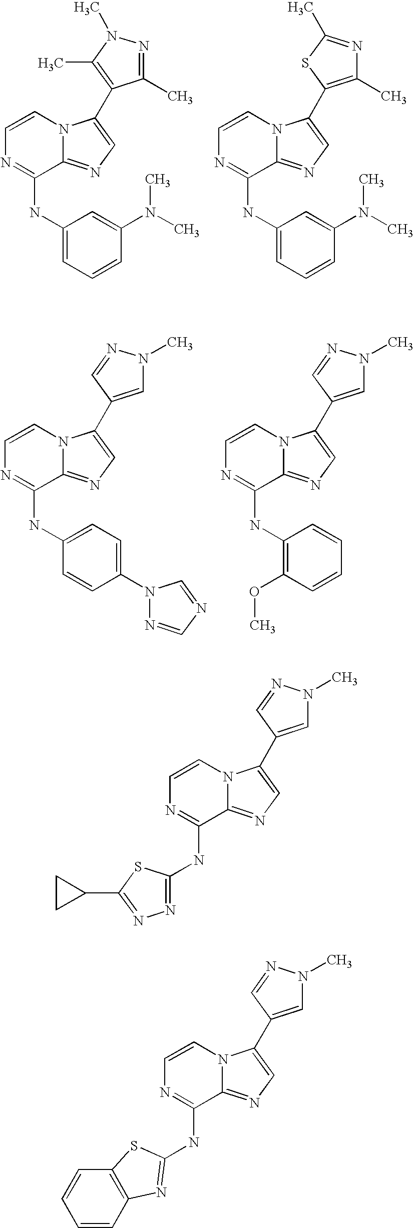

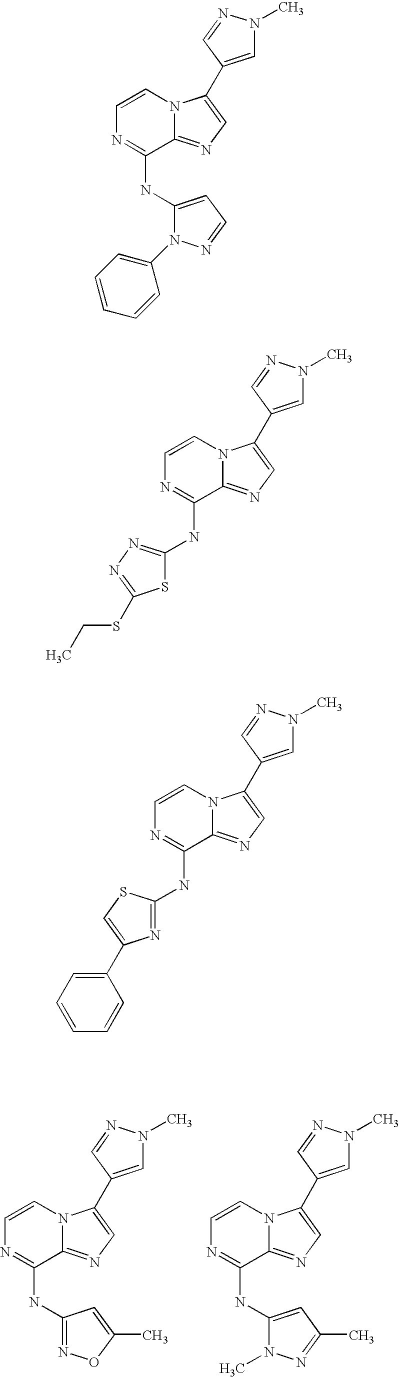

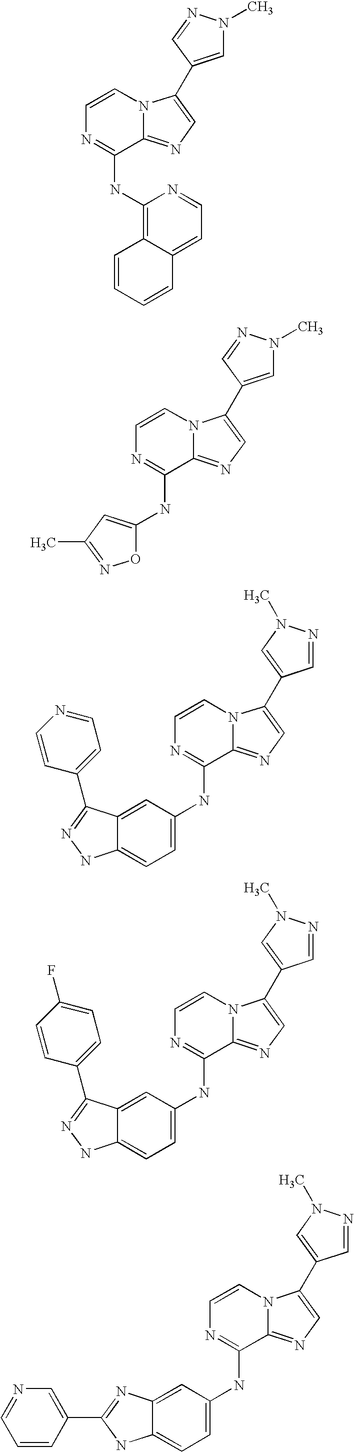

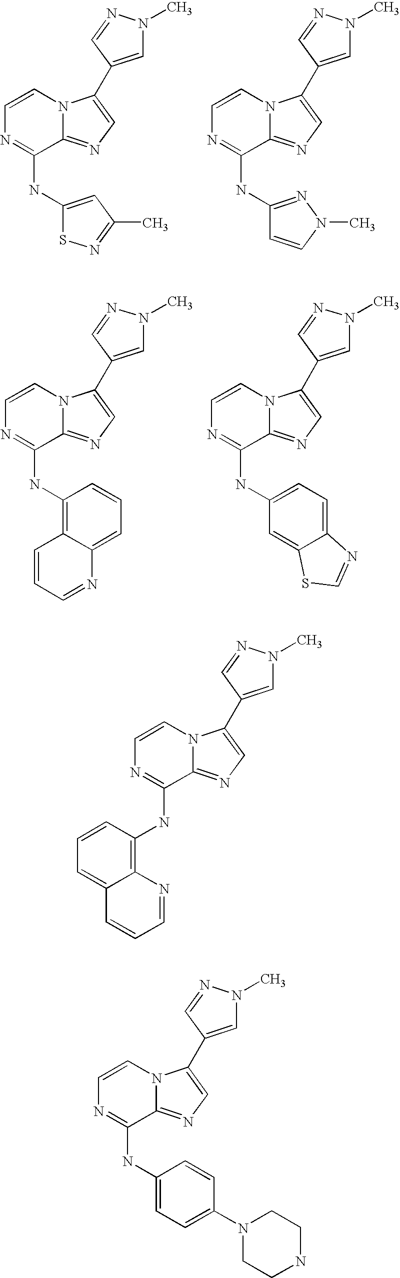

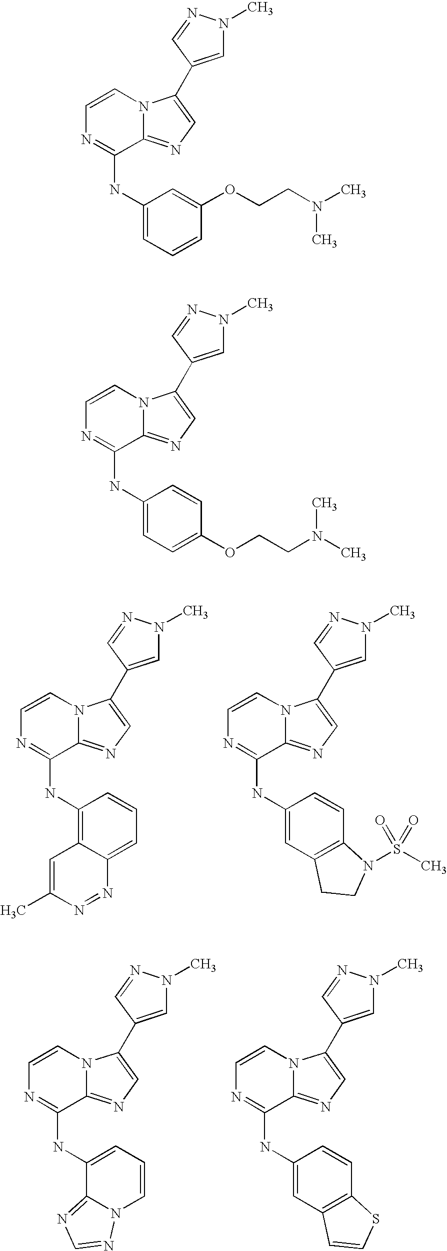

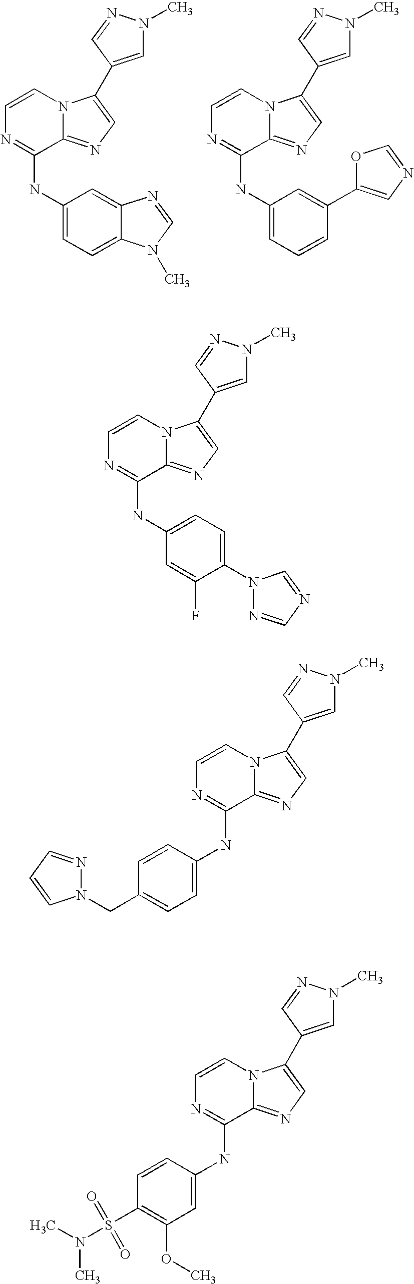

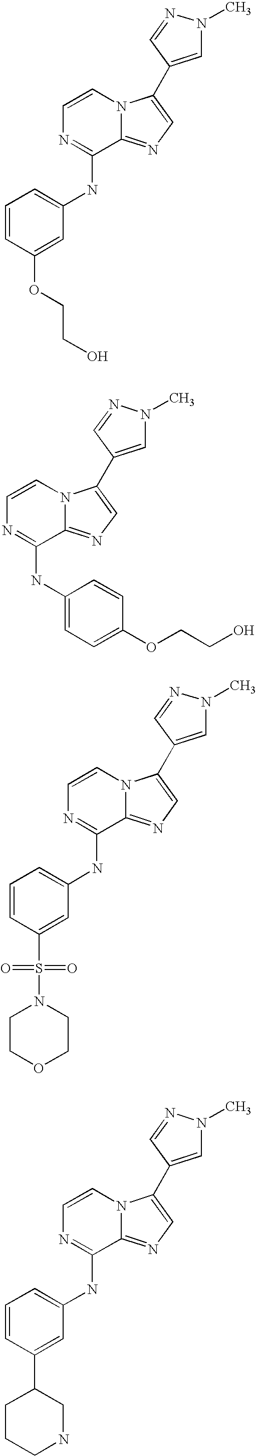

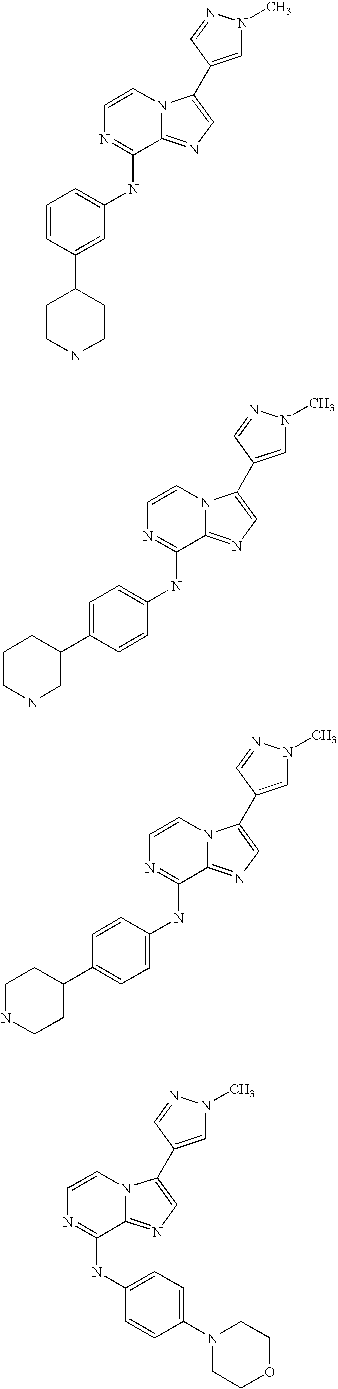

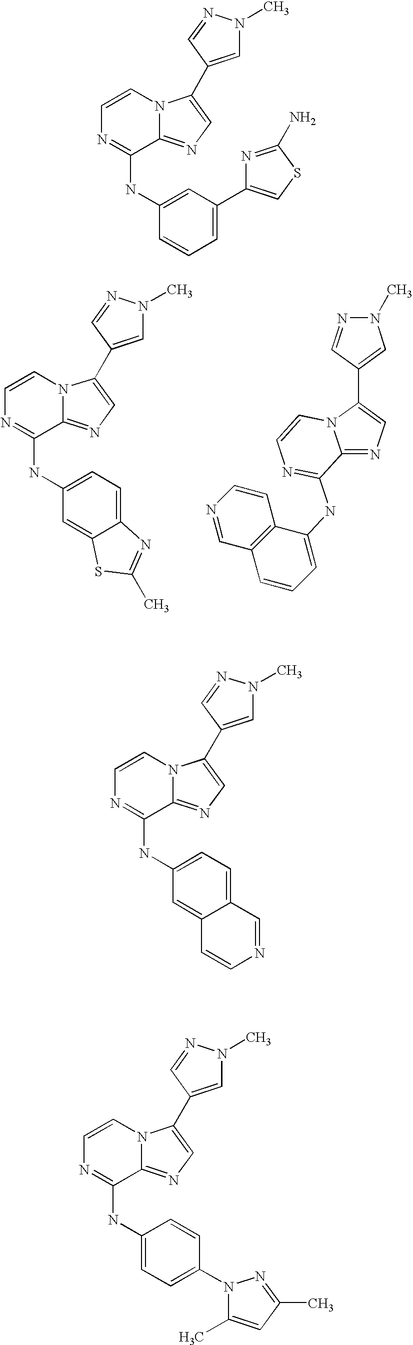

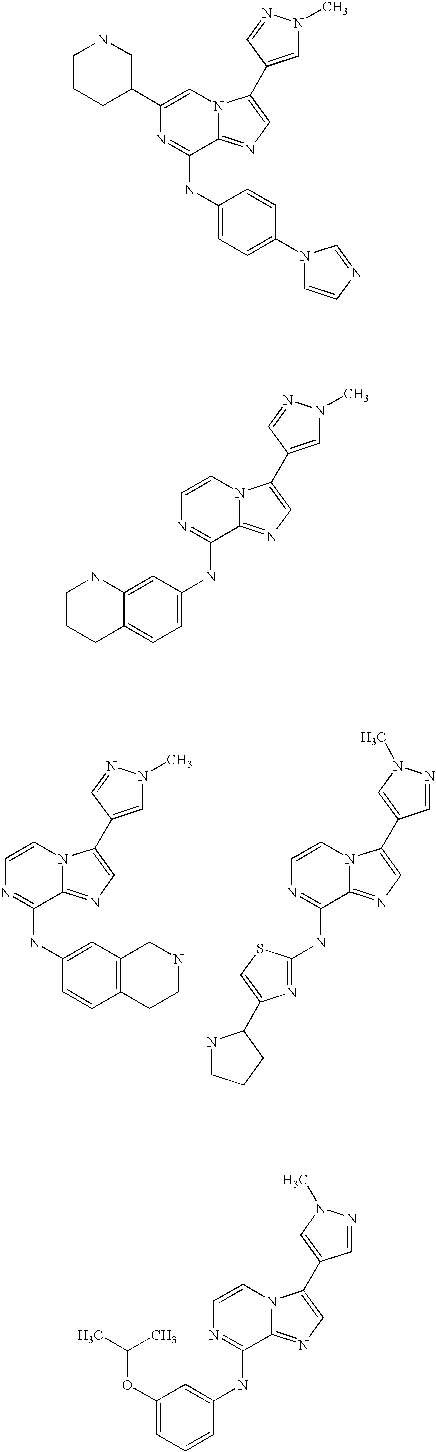

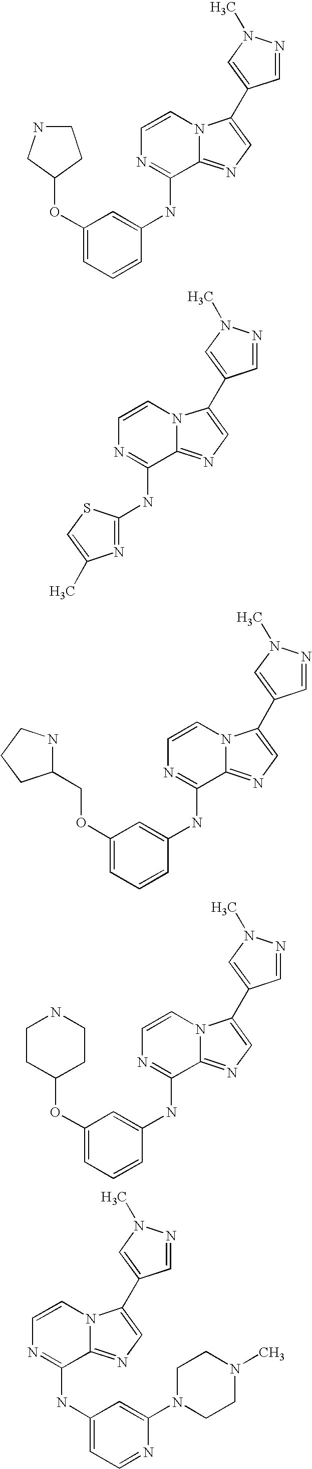

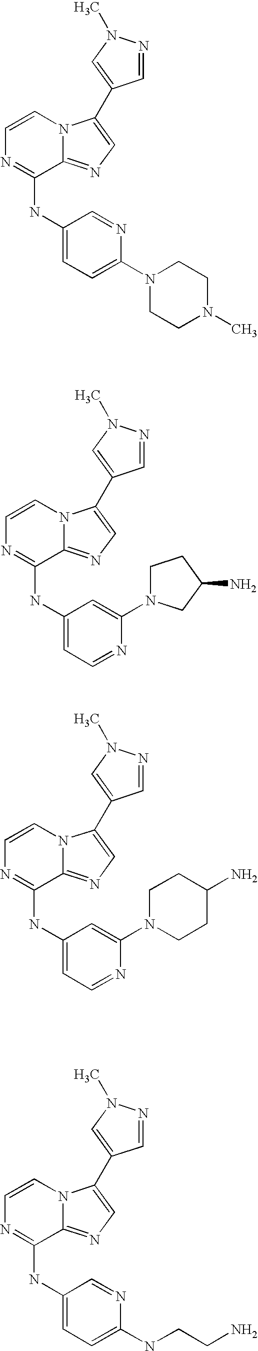

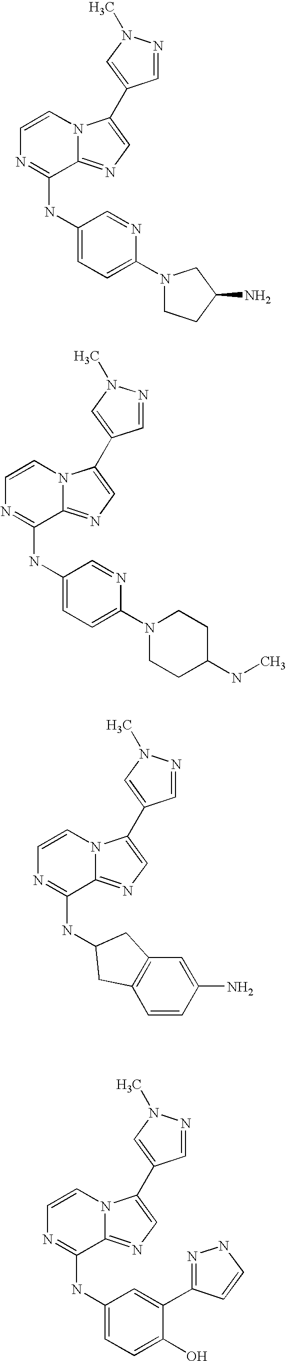

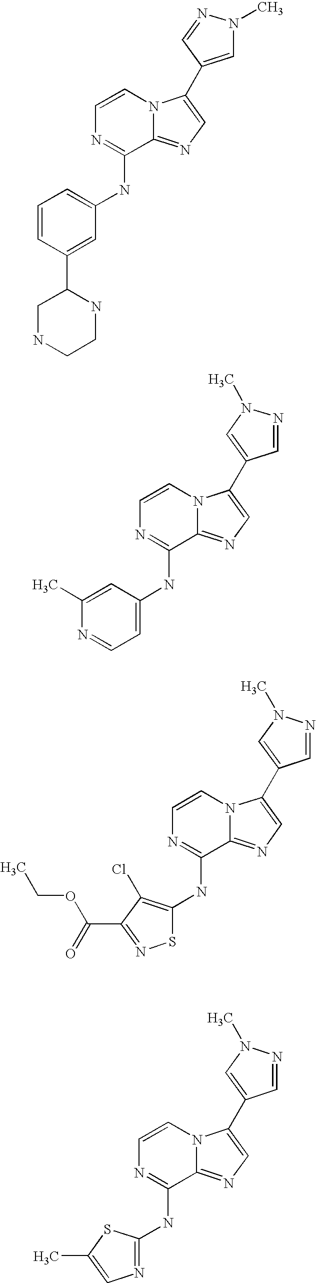

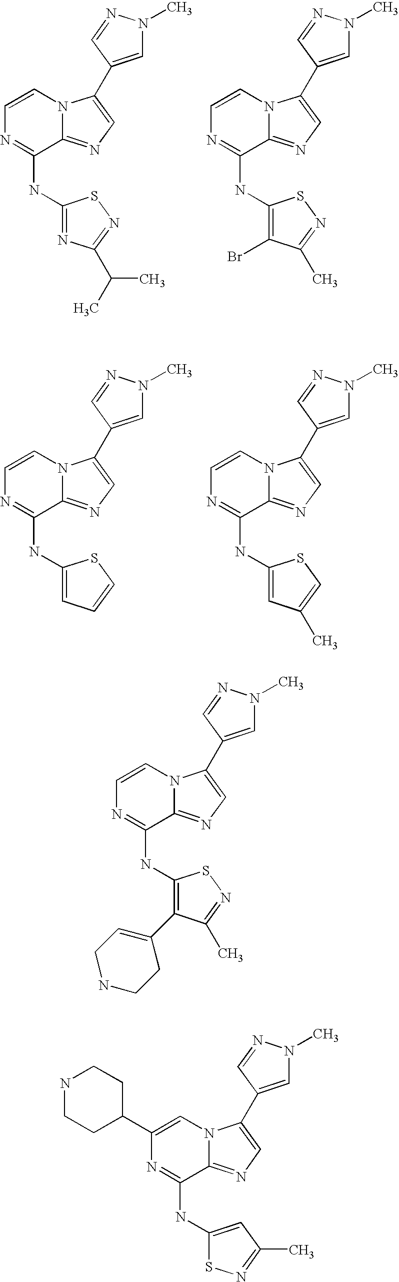

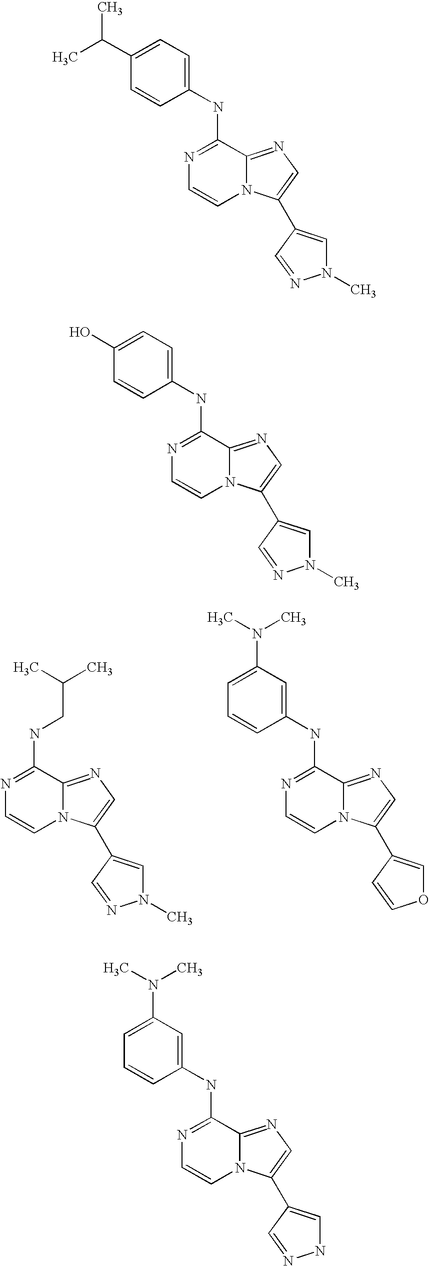

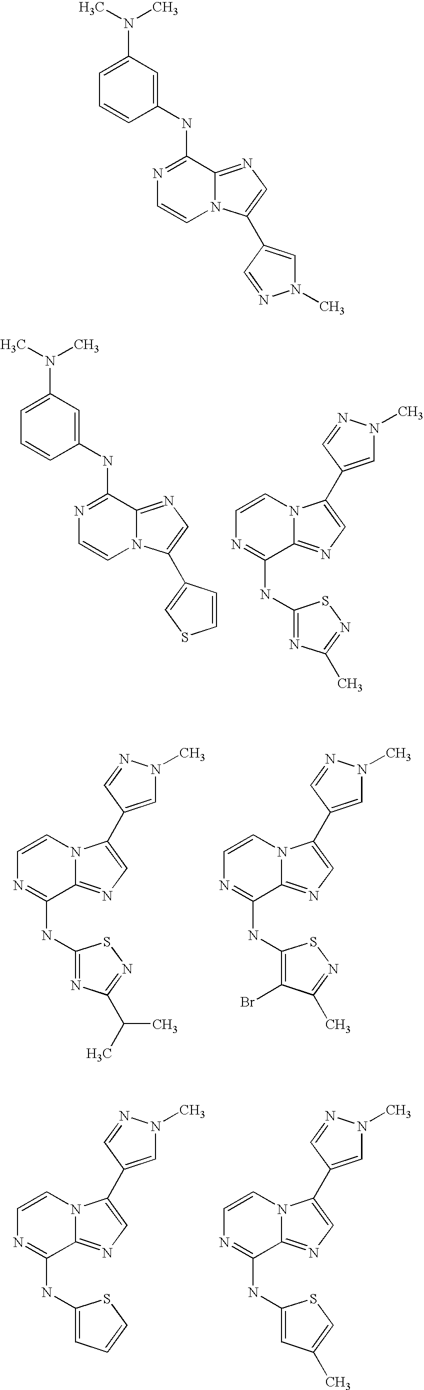

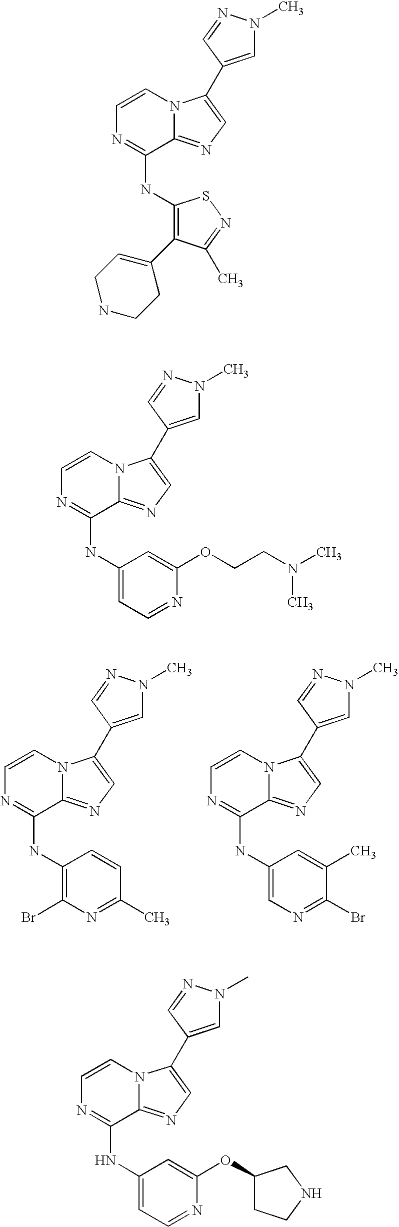

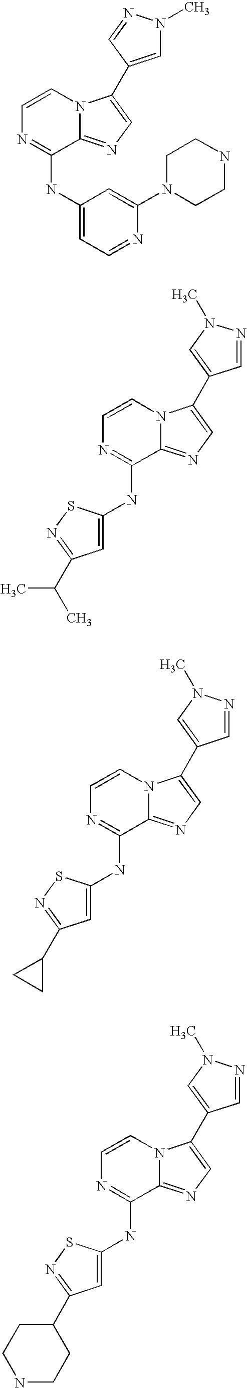

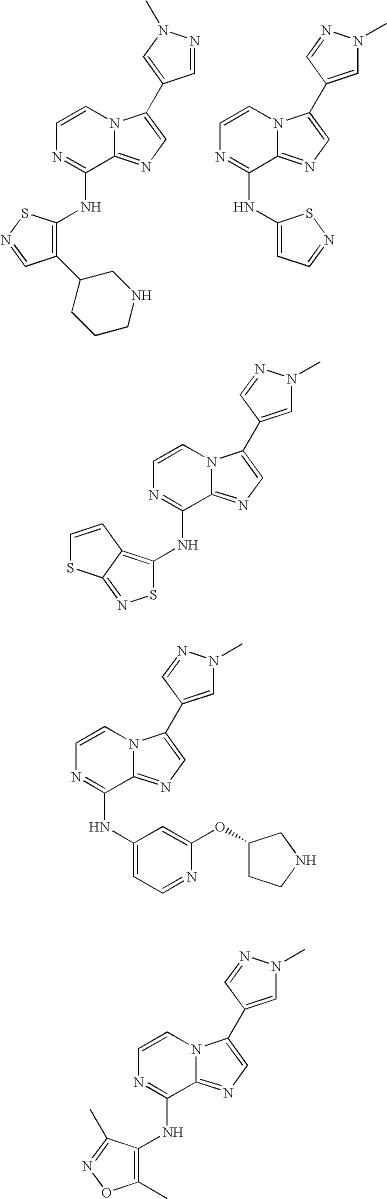

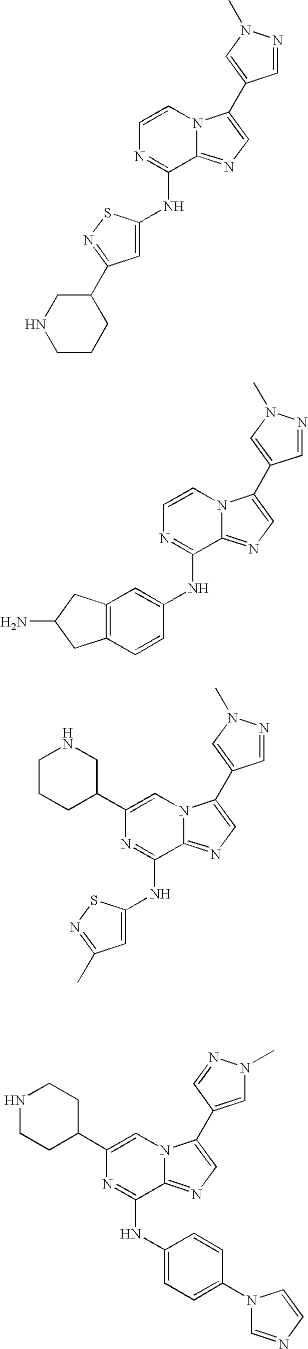

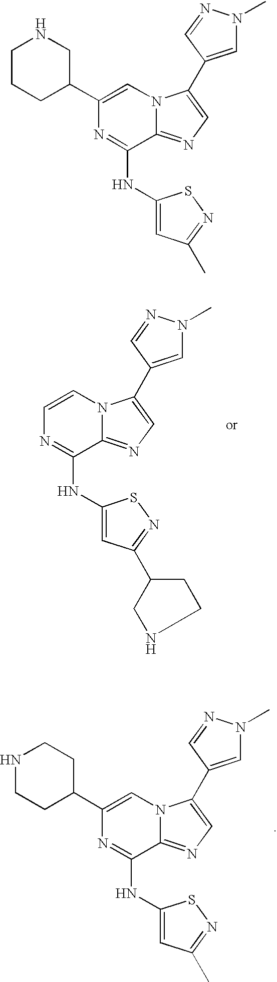

or a pharmaceutically acceptable salt, solvate, ester or prodrug thereof.
Such compounds of Formula G in (g) are disclosed in WO2007/058873, filed Nov. 8, 2006, the content of which is incorporated herein by reference in its entirety;

h. A compound of the formula:

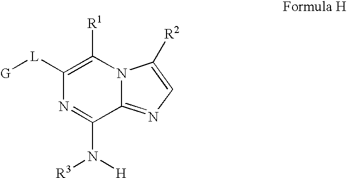

or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷,

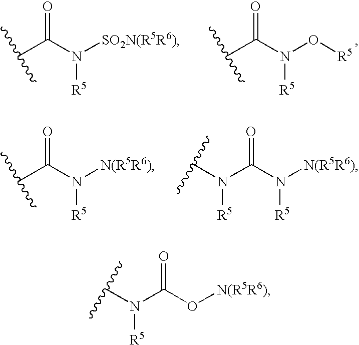

alkyl substituted with 1-6 R⁹ groups which groups can be the same or different with each R⁹ being independently selected,

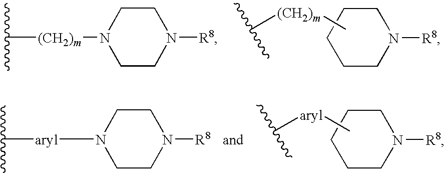

wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,

### —N(R⁵)C(O)R⁷ and —N(R⁵)C(O)NR⁵R⁶;

R³ is selected from the group consisting of H, alkyl, aryl, heteroaryl, heterocyclyl, aralkyl, heteroarylalkyl, cycloalkylalkyl, heterocyclylalkyl,

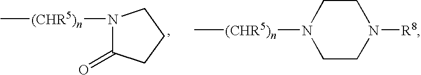

—(CHR⁵)ₙ—OR⁶, —S(O₂)R⁶, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₘ—NR⁸, and

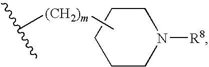

wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
   —S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
   —C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
   m is 0 to 4; and
   p is 0-3.
      Such compounds of Formula H in (h) are disclosed in WO 2008/054749, filed Oct. 29, 2007, the entire contents of which are incorporated herein by reference thereto;

i. A compound of Formula I:

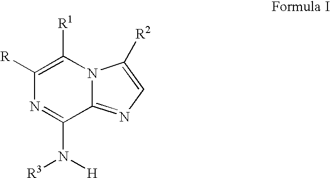

or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
   —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
   —NR⁵R⁶ (wherein the R⁵ and R⁶, together with the N of said
   —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
   —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
   X is selected from the group consisting of —NR⁵R⁶, —OR⁵, —SO—R⁵, —SR⁵, SO₂R⁵, heteroaryl, heterocyclyl and aryl, wherein said heteroaryl or aryl can be unsubstituted or substituted with one or more moieties, independently selected from the group consisting of —O-alkyl, alkyl, halo, or NR⁵R⁶;
   R⁷ and R⁸ are each independently hydrogen, alkyl, heterocyclyl, aryl, heteroaryl or cycloalkyl;
   R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cyclenyl, heterocyclylalkoxyl, —S-alkylheterocyclyl, heterocyclyl, heterocyclenyl, alkylN(alkyl)₂, alkylNH(alkyl), alkylN(alkenyl)₂, -alkylN(alkoxyl)₂, -alkyl-SH, hydroxyalkyl, trihaloalkyl, dihaloalkyl, monohaloalkyl, wherein each of said alkyl, alkenyl, alkoxyalkyl, -alkyl-S-alkyl, aminoalkyl, aryl, heteroaryl, heterocyclenyl, heterocycloalkyl, cycloalkyl, cyclenyl, heterocyclylalkoxyl, —S-alkylheterocyclyl, heterocyclyl, heterocyclenyl, alkylN(alkyl)₂, alkylNH(alkyl), alkylN(alkenyl)₂, -alkylN(alkoxyl)₂, -alkyl-SH, hydroxyalkyl, trihaloalkyl, dihaloalkyl, monohaloalkyl can be unsubstituted or substituted with one or more moieties independently selected from the group consisting of alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
   R⁶ is selected from the group consisting of hydrogen, alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, aminoalkyl, -alkyl-OC(O)alkyl, -alkylOC(O)cycloalkyl, -alkylOC(O)aryl, -alkylOC(O)aralkyl, -alkylOC(O)NR⁵aryl, -alkylOC(O)NR⁵alkyl, -alkylOC(O)NR⁵heterocyclyl, -alkylOC(O)NR⁵heteroaryl -alkylOC(O)NR⁵cycloalkyl, -alkylOC(O)heterocyclyl, alkylC(O)OH, alkylC(O)Oalkyl, -alkylC(O)Ocycloalkyl, -alkylC(O)Oaryl, -alkylC(O)Oaralkyl, -alkylC(O)ONR⁵aryl, -alkylC(O)ONR⁵alkyl, -alkylC(O)ONR⁵heterocyclyl, -alkylC(O)ONR⁵heteroaryl -alkylC(O)ONR⁵cycloalkyl, -alkylC(O)Oheterocyclyl, alkylC(O)OH, and alkylC(O)Oalkyl wherein each of said aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, -alkyl-OC(O)alkyl, -alkylOC(O)cycloalkyl, -alkylOC(O)aryl, -alkylOC(O)aralkyl, -alkylOC(O)NR⁵aryl, -alkylOC(O)NR⁵alkyl, -alkylOC(O)NR⁵heterocyclyl, -alkylOC(O)NR⁵heteroaryl -alkylOC(O)NR⁵cycloalkyl, -alkylOC(O)heterocyclyl, alkylC(O)OH, alkylC(O)Oalkyl, -alkylC(O)Ocycloalkyl, -alkylC(O)Oaryl, -alkylC(O)Oaralkyl, -alkylC(O)ONR⁵aryl, -alkylC(O)ONR⁵alkyl, -alkylC(O)ONR⁵heterocyclyl, -alkylC(O)ONR⁵heteroaryl -alkylC(O)ONR⁵cycloalkyl, -alkylC(O)Oheterocyclyl, alkylC(O)OH, and alkylC(O)Oalkyl, can be unsubstituted or substituted with one or more moieties independently selected from the group consisting of alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, aminoalkyl, amino, aminodialkyl, aminocycloalkyl, halo, trihaloalkyl, dihaloalkyl, and monohaloalkyl;
   further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cyclenylalkyl, cycloalkylalkyl, heteroarylalkyl, heterocyclenylalkyl, heterocycloalkylalkyl, heteroaryl, aryl, cyclenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, heterocyclenyl, halo, trihaloalkyl, dihaloalkyl, CN and monohaloalkyl.
      Such compounds of Formula I in (i) are disclosed in PCT US 2008/007295 filed Jun. 11, 2008;

j. A compound having the formula:

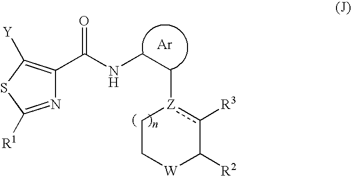

or a pharmaceutically acceptable salt, solvate, ester, prodrug or stereoisomer thereof, wherein the dashed lines indicate an optional and additional bond, and wherein:

R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;

R² is —H, -alkyl, —NH₂ or —CH₂NH₂;

R³ is —H, -alkyl, —NH₂ or —CH₂NH₂;

each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;

R⁵ is —H, -alkyl, -aryl, -heteroaryl, —NHOH;

R⁶ is —H, -alkyl or —CF₃;

R⁷ is —H, —OH, —C₁-C₆ alkyl, —O—(C₁-C₆ alkyl), or —CF₃;

R⁸ is —H, alkyl, aryl, heterocyclyl, heteroaryl or cycloalkyl;

Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen

W is —NH— or —C(R⁴)₂—, wherein both R⁴ groups and the carbon atom to which they are attached can combine to form a five to seven membered heterocyclyl or heteroaryl group;

Y is —H, -halo, -alkyl or —CN;

Z is —CR⁷— or —N—, when the optional additional bond is absent, and —C— when the optional additional bond is present;

n is an integer ranging from 0 to 2;

Such compounds of Formula J in (j) are disclosed in WO 2008/054749, filed Oct. 29, 2007; and

(k) A compound of Formula K:

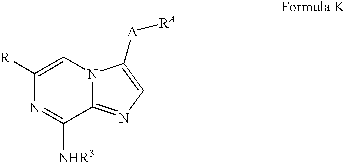

or a pharmaceutically acceptable salt, solvate, ester, prodrug or stereoisomer thereof, wherein:

R is H, halo or alkyl;

R³ is heteroaryl-X, wherein X is heterocyclylalkyl- wherein said heterocyclyl can be unsubstituted or optionally substituted with 1-4 alkyl moieties;

A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;

R^{A} is —(CH₂)₁₋₄-heteroaryl,

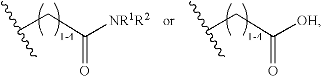

wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
   R¹ and R² together with the N to which each is attached, form a heterocyclic group selected from the group consisting of

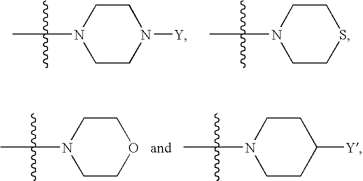

wherein
Y is alkoxyalkyl, hydroxyalkyl, dialkylaminoalkyl or alkyl, further wherein Y′ is hydroxyl.
   Such compounds of Formula K in paragraph (k) are disclosed in U.S. provisional patent application, 61/024,010, filed Jan. 28, 2008.

### BRIEF DESCRIPTION OF THE DRAWINGS

FIG. 1 is a FACS (Flow Cytometric Analysis) analysis of HCT-116 colon cancer cells. Cells were treated with 1000 nM Aurora kinase inhibitor (Compound X) (WO 2008/057512, filed Nov. 6, 2007, Example 4-3 and Claim 70 and it is represented in Formula H and Table 2, column 2, row 16 of the present application) for the indicated times, at which time, drug was washed off and replaced with new media. FACS was analyzed after a total of 72 hours. Exposure for less than 24 hours were insufficient to induce endoreduplication (>4N DNA content), while drug exposure for 24, 48, or 72 hours resulted in the accumulation of cells that underwent endoreduplication.

FIG. 2 is a FACS analysis of HCT-116 colon cancer cells treated with 1000 nM Aurora kinase inhibitor (Compound X) for the indicated times, at which time, drug was washed off and replaced with new media. FACS was analyzed after a total of 24 hours. Twenty four hour treatment was sufficient to induce endoreduplication, however less exposure time was insufficient to induce endoreduplication

FIG. 3 is a FACS analysis of HCT-116 colon cancer cells treated for 16 hours with DMSO. The cells were then exposed to DMSO or 1000 nM Aurora kinase inhibitor (Compound X) for 4, 8, or 24 hours at which time the media was changed. All cells were analyzed at the end of 24-hours.

FIG. 4 is a FACS analysis of HCT-116 colon cancer cells treated for 16 hours with 5 nM taxotere. Cells were then exposed to DMSO or 1000 nM Aurora kinase inhibitor (Compound X) for 4, 8, and 24 hours at which time the media was changed. All cells were analyzed at the end of 24-hours. Taxotere followed by Aurora kinase inhibitor (Compound X) induced endoreduplication. Endoreduplication was observed even when Aurora kinase inhibitor (Compound X) exposure was as little as 4 hours.

FIG. 5 is a FACS analysis of HCT-116 colon cancer cells pre-treated with nocodazole for 16 hours. Cells were then exposed to DMSO or 1000 nM Aurora kinase inhibitor (Compound X) for 4, 8, and 24 hours at which time the media was changed. All cells were analyzed at the end of 24-hours. Nocodazole followed by 24 hour exposure to Aurora kinase inhibitor (Compound X) induced endoreduplication, however a 4- or 8-hour exposure to Aurora inhibitor (Compound X) was insufficient to induce endoreduplication.

FIG. 6 is a FACS analysis of HCT-116 colon cancer cells treated with an Aurora kinase inhibitor (Compound X) given at the same time as taxotere, 4 hour exposure was not sufficient to induce endoreduplication and 24 hour exposure was needed to induce endoreduplication.

FIG. 7 is a FACS analysis of HCT-116 colon cancer cells treated for 16 hours with 10 nM Ispinesib (KSP inhibitor). Cells were then exposed to DMSO or 1000 nM Aurora kinase inhibitor (Compound X) for 4, 8, and 24 hours at which time the media was changed. All cells were analyzed at the end of 24-hours. Ispinesib followed by Aurora kinase inhibitor (Compound X) induced endoreduplication. Endoreduplication was observed even when Aurora kinase inhibitor (Compound X) exposure was as little as 4 hours.

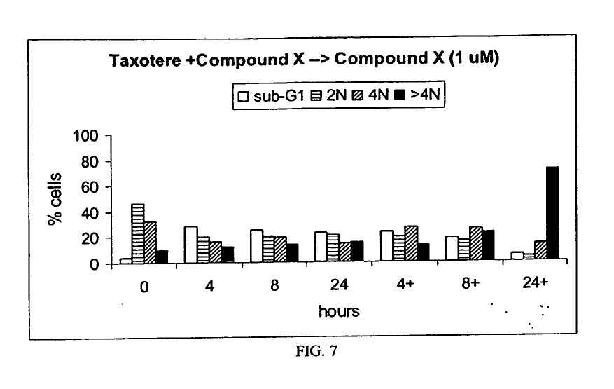

FIG. 8 is a FACS (Flow Cytometric Analysis) analysis of HCT-116 colon cancer cells. Cells were treated with 25 nM Aurora kinase inhibitor (Compound Z) (PCT US2008/007295, filed Jun. 11, 2008, Example 76-2 and Claim 25, row 7, column 4 and it is represented in Table 13, compound 76-2 of the present application) for the indicated times, at which time, drug was washed off and replaced with new media. FACS was analyzed after a total of 72 hours. Exposure for less than 24 hours were insufficient to induce endoreduplication (>4N DNA content), while drug exposure for 24, 48, or 72 hours resulted in the accumulation of cells that underwent endoreduplication.

FIG. 9 is a FACS analysis of HCT-116 colon cancer cells treated with 25 nM Aurora kinase inhibitor (Compound Z) for the indicated times, at which time, drug was washed off and replaced with new media. FACS was analyzed after a total of 24 hours. Twenty four hour treatment was sufficient to induce endoreduplication, however less exposure time was insufficient to induce endoreduplication

FIG. 10 is a FACS analysis of HCT-116 colon cancer cells treated for 16 hours with DMSO. The cells were then exposed to DMSO or 25 nM Aurora kinase inhibitor (Compound Z) for 4, 8, or 24 hours at which time the media was changed. All cells were analyzed at the end of 24-hours.

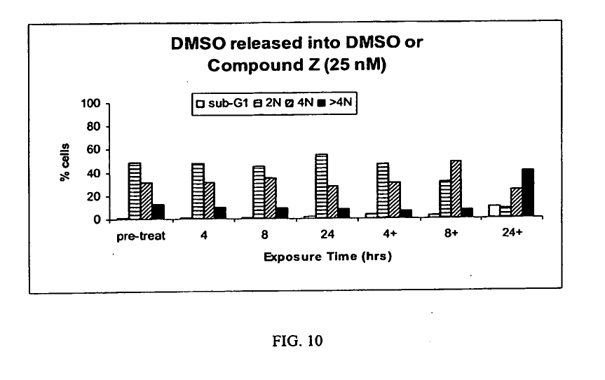

FIG. 11 is a FACS analysis of HCT-116 colon cancer cells treated for 16 hours with 5 nM taxotere. Cells were then exposed to DMSO or 25 nM Aurora kinase inhibitor (Compound Z) for 4, 8, and 24 hours at which time the media was changed. All cells were analyzed at the end of 24-hours. Taxotere followed by Aurora kinase inhibitor (Compound Z) induced endoreduplication. Endoreduplication was observed even when Aurora kinase inhibitor (Compound Z) exposure was as little as 4 hours.

FIG. 12 is a FACS analysis of HCT-116 colon cancer cells treated with an Aurora kinase inhibitor (Compound Z) given at the same time as taxotere, 4 hour exposure was not sufficient to induce endoreduplication and 24 hour exposure was needed to induce endoreduplication.

FIG. 13 is a FACS analysis of HCT-116 colon cancer cells treated for 16 hours with 10 nM Ispinesib (KSP inhibitor). Cells were then exposed to DMSO or 25 nM Aurora kinase inhibitor (Compound Z) for 4, 8, and 24 hours at which time the media was changed. All cells were analyzed at the end of 24-hours. Ispinesib followed by Aurora kinase inhibitor (Compound Z) induced endoreduplication. Endoreduplication was observed even when Aurora kinase inhibitor (Compound Z) exposure was as little as 4 hours.

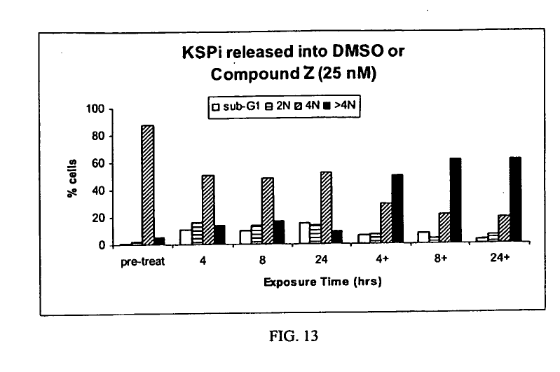

FIG. 14 is a FACS analysis of HCT-116 colon cancer cells treated with an Aurora kinase inhibitor (Compound Z) given at the same time as Ispinesib (KSP inhibitor) taxotere, 4 hour exposure was not sufficient to induce endoreduplication and 24 hour exposure was needed to induce endoreduplication.

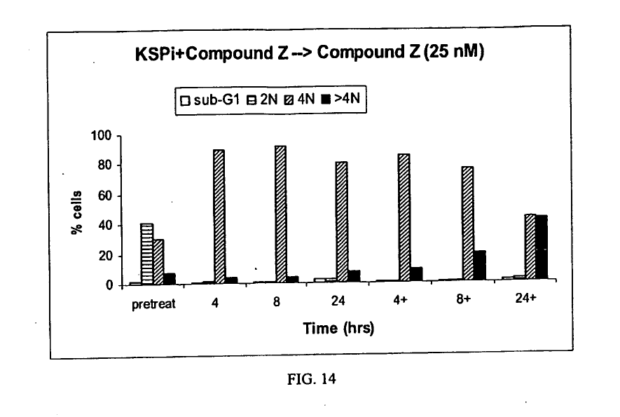

Similar to the above-described Figures (Drawings), other experimental results were obtained when, for example, the Aurora kinase inhibitor was changed from Compound X or Z to other aurora kinase inhibitors disclosed elsewhere in this present application.

### DETAILED DESCRIPTION

The method of treatment using sequential administration of at least one anti-mitotic agent followed by at least one aurora kinase inhibitor described herein to a subject in need thereof is exemplified below.

In one embodiment, the present invention discloses a pharmaceutical composition for treating or ameliorating cancer comprising at least one anti-mitotic agent selected from the group consisting of Taxanes, paclitaxel, docetaxel, Cenp-E Inhibitor (such as for example GSK-923295 from Glaxo Smithkline), Abraxane, Epothilone, Monastrol, as well as KSP inhibitors such as, for example, Ispinesib SB-715992 (from Cytokinetics, South San Francisco, Calif.) and the compounds of Formulas A-D in paragraphs a-d below:

a. A compound represented by the structural Formula A:

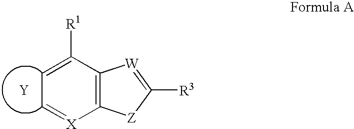

or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
   each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
   or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
   each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
   each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
   each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰) NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰)C(O)OR¹¹, and —NR¹⁰C(O)R⁴⁰;
   or two R⁸ groups, when attached to the same carbon atom, are optionally taken together with the carbon atom to which they are attached to form a C═O or a C═S group;
   each R⁹ is independently selected from the group consisting of H, alkyl, alkoxy, OH, CN, halo, —(CR¹⁰R¹¹)⁰⁻⁴NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
   each R¹⁰) is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
   each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
   each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
   R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
      (see WO 2006/098962, filed Mar. 7, 2006);

b. A compound represented by the structural Formula B:

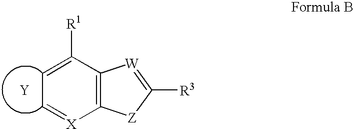

or a pharmaceutically acceptable salt, solvate or ester thereof, wherein:

ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;

W is N or C(R¹²);

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;

each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;

or two R²s on the same carbon atom are optionally taken together with the carbon atom to which they are attached to form a C═O, a C═S or an ethylenedioxy group;

R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷) NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵,

### —C(O)N(R⁷)(CR⁴⁰R⁴¹)₁₋₅—C(O)—NR⁴R⁵,

—C(O)N(R⁷)(CR⁴⁰R⁴¹)₁₋₅—OR⁷, —C(S)NR⁷(CH₂)₁₋₅NR⁴R⁵, and —C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;

each of R⁴ and R⁵ is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;

each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰) NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;

or two R⁸ groups, when attached to the same carbon, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R^{H} alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,

—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)NR⁴R⁵, —NR⁴C(S)R⁵, —NR⁴C(O)OR⁷, NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

R⁴⁰ and R⁴¹ can be the same or different, each being independently selected from the group consisting of H, alkyl, aryl, heteroaryl, heterocyclyl, heterocyclenyl, cycloalkyl and cycloalkenyl;

each R⁴² is independently selected from the group consisting of halo, alkyl, cycloalkyl, heterocyclyl, aryl, heteroaryl, —NO₂, —(Cᵣ—C₆ alkyl)-OR¹⁰,

### —CN, —NR¹⁰R¹¹, —C(O)R¹⁰, —C(O)OR¹⁰, —C(O)NR¹⁰R¹¹, —CF₃, —OCF₃,

### —N(R¹⁰)C(O)R¹¹,and —NR¹⁰C(O)OR¹¹;

with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹; (see WO2006/098961, filed Mar. 7, 2006);

c.

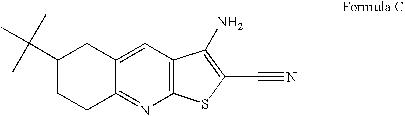

or a pharmaceutically acceptable salt, solvate, or ester thereof;
(see U.S. Pub. No. 2006/0258699, filed Mar. 7, 2006) and;

d. A compound of Formula D

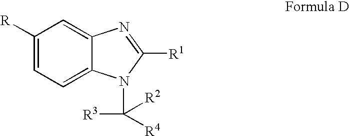

or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O))NH(cycloalkyl), —C(═O)N(alkyl)₂, —C(O)H, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
   Compounds represented in the present application by Formula (d) are disclosed in PCT US2008/006472, filed May 21, 2008;
   further comprising one or more of a second compound, wherein said second compound is an aurora kinase inhibitor selected from the group consisting of compounds represented by Formulas E-K:

e. A compound represented by the structural Formula E:

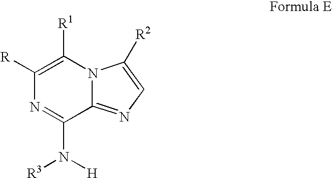

or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
   —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
   —NR⁵R⁶ (wherein the R⁵ and R⁶, together with the N of said
   —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
   —CH₂OR⁵, —S(O)R⁵, —S(O₂)R⁵, —CN, —CHO, —SR⁵, —C(O)OR⁵, —C(O)R⁵, heteroaryl and heterocyclyl;
      R³ is H, alkyl, cycloalkyl, heterocyclyl, aryl or heteroaryl, wherein:
   said alkyl shown above for R³ can be unsubstituted or substituted with one or more moieties which can be the same or different each moiety being independently selected from the group consisting of —OR⁵, alkoxy, heteroaryl, and —NR⁵R⁶;
   said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
      —S(O₂)R⁵; and
   said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
      R⁵ is H, alkyl, aminoalkyl, aryl, heteroaryl, heterocyclyl or cycloalkyl; and
      R⁶ is H, alkyl, aryl, arylalkyl, heteroaryl, heterocyclyl or cycloalkyl;
      further wherein in any —NR⁵R⁶ in Formula I, said R⁵ and R⁶ can optionally be joined together with the N of said —NR⁵R⁶ to form a cyclic ring;
      (see WO07/058,942 filed Nov. 8, 2006);

f. A compound represented by the structural formula:

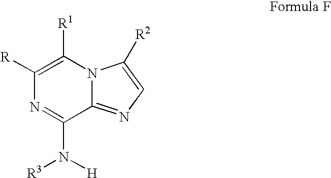

or a pharmaceutically acceptable salt, solvate, ester or prodrug thereof, wherein:

R is selected from the group consisting of H, halogen, aryl, heteroaryl, cycloalkyl, arylalkyl, heterocyclyl, heterocyclylalkyl, alkenyl, alkynyl,

### —C(O)R⁷,

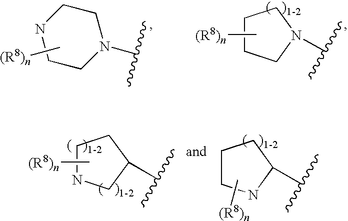

wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;

R¹ is H, halogen or alkyl;

R² is alkyl;

R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₙ—NR⁸, —(CHR⁵)ₙ—CH(aryl)₂,

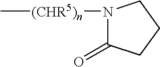

and

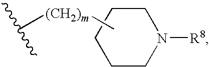

wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;

R⁵ is H or alkyl;

R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and

### —N(R⁵)C(O)NR⁵R⁶;

R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,

—S(O₂)NR⁵R⁶, —N(R⁵)S(O₂)R⁷, —N(R⁵)C(O)R⁷ and —N(R⁵)C(O)NR⁵R⁶;

R⁸ is selected from the group consisting of R⁶, —C(O)NR⁵R⁶,

—S(O₂)NR⁵R⁶, —C(O)R⁷, —C(O₂)R⁶, —S(O₂)R⁷ and —(CH₂)-aryl;

R⁹ is selected from the group consisting of halogen, CN, NR⁵R⁶,

—C(O₂)R⁶, —C(O)NR⁵R⁶, —OR⁶, —C(O)R⁷, —SR⁶, —S(O₂)R⁷, —S(O₂)NR⁵R⁶,

### —N(R⁵)S(O₂)R⁷, —N(R⁵)C(O)R⁷ and —N(R⁵)C(O)NR⁵R⁶;

m is 0 to 4;

n is 1-4; and

p is 0-3;

(see WO2004/026877, filed Sep. 19, 2003);

g. A compound selected from the compounds of the formulas:

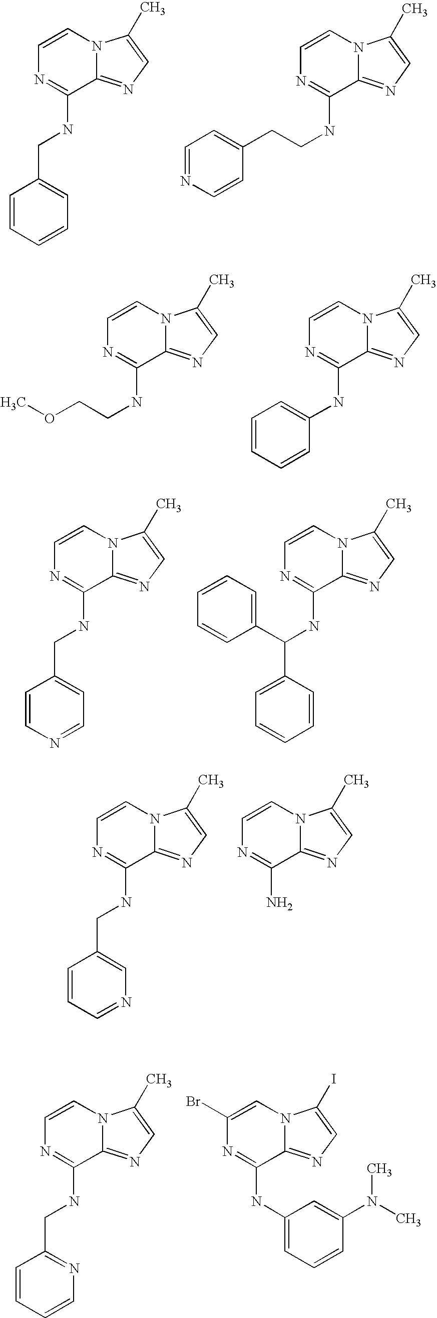

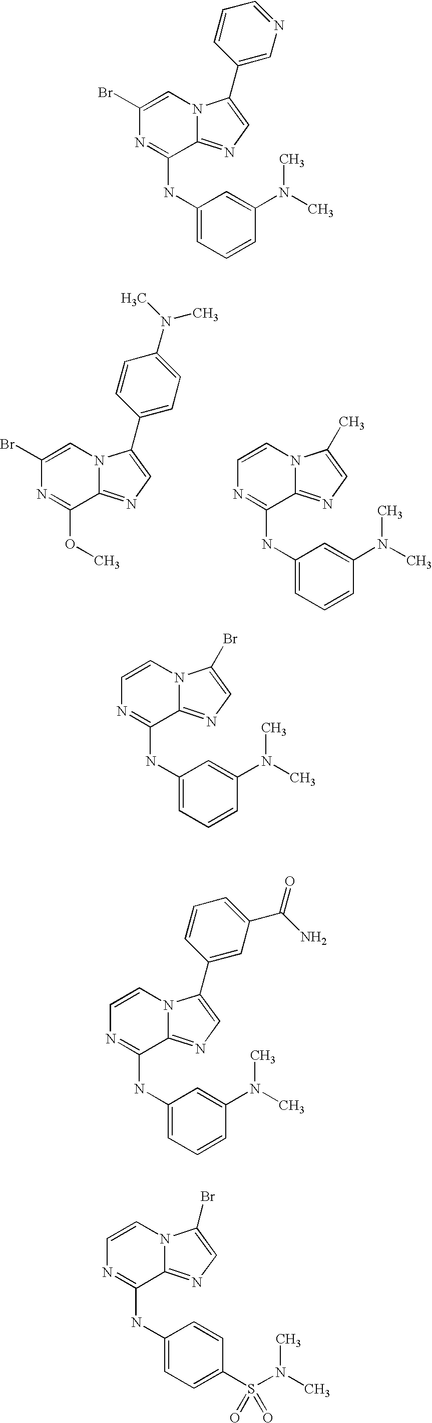

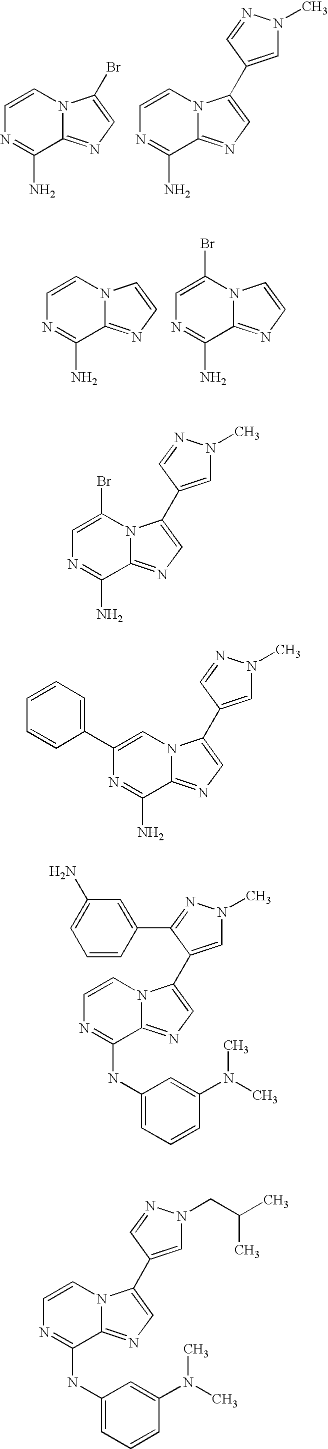

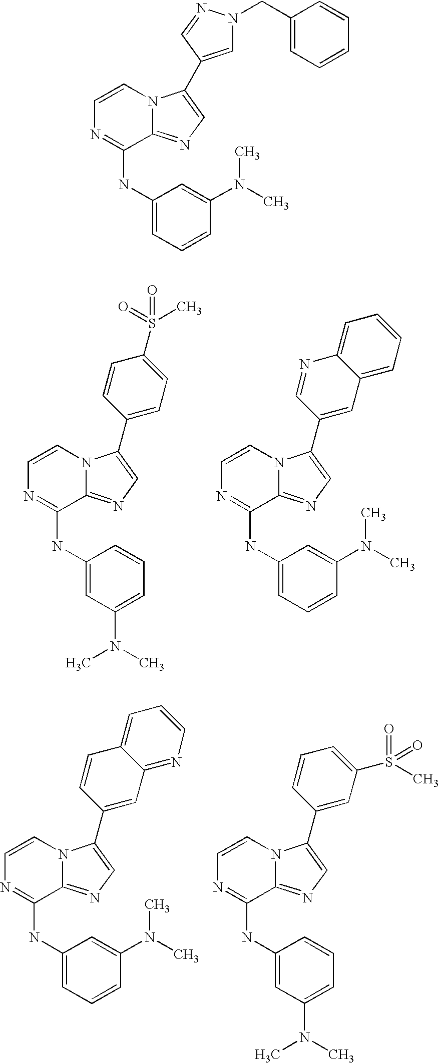

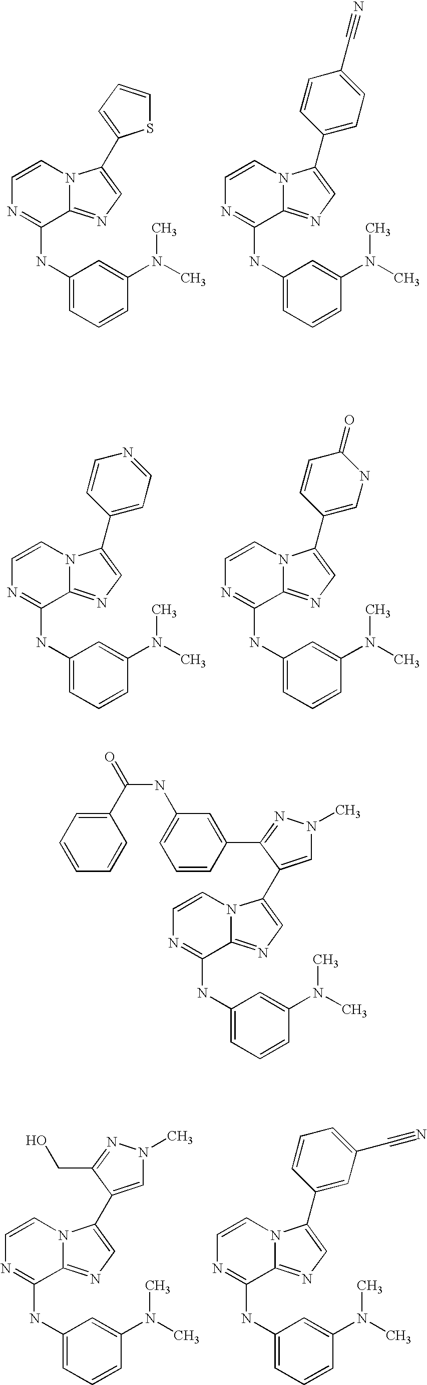

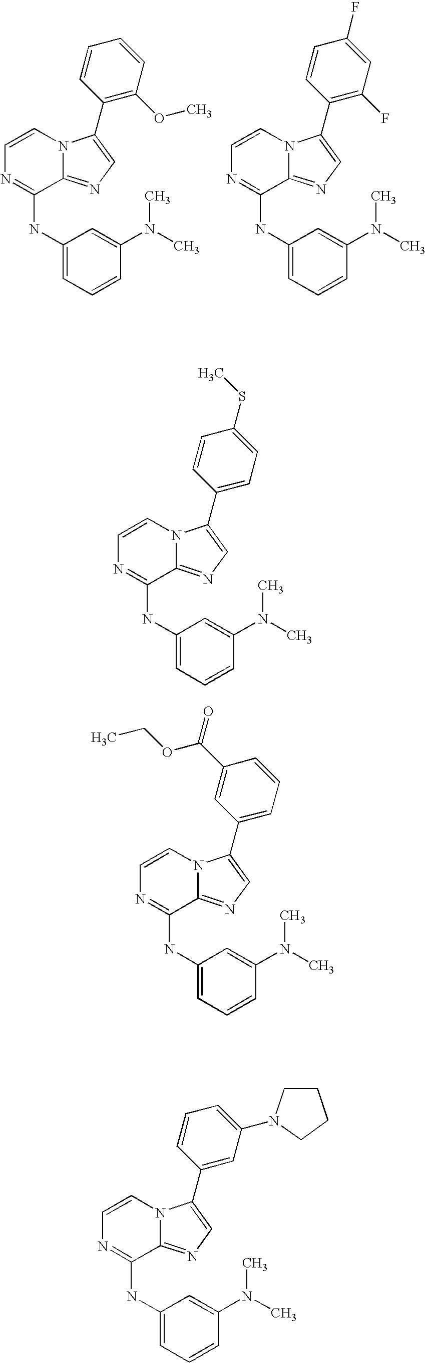

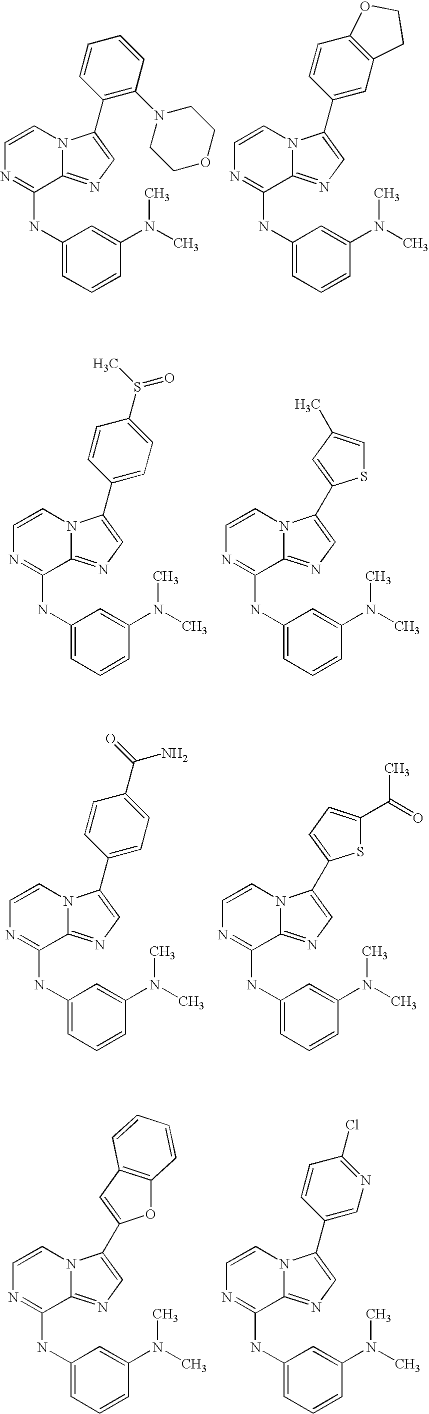

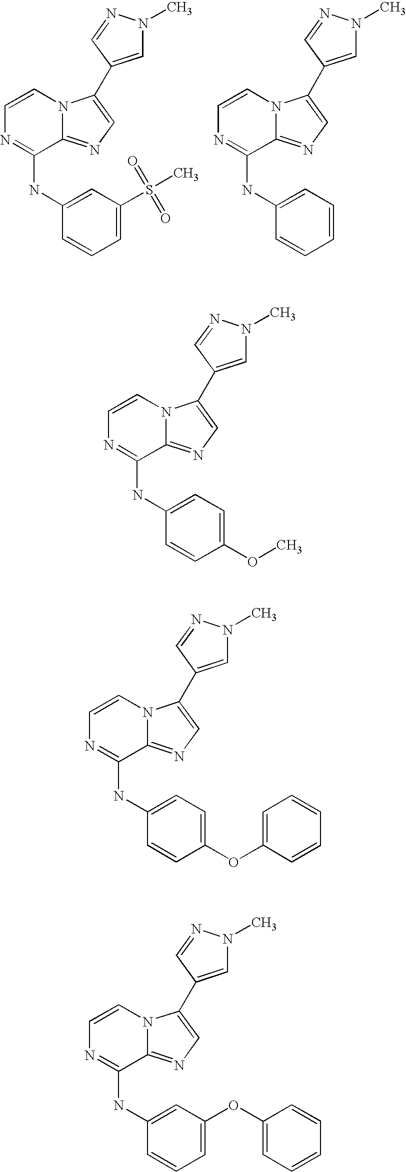

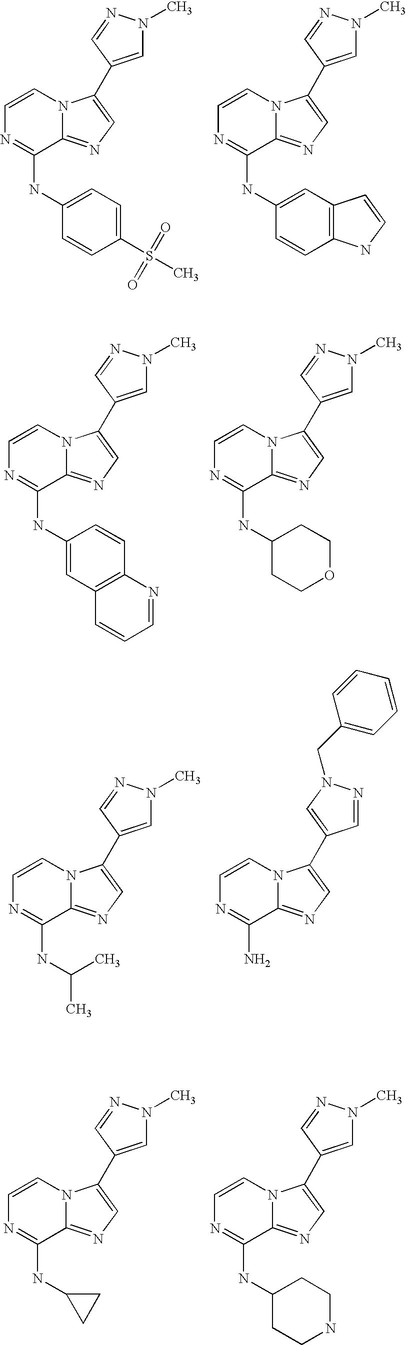

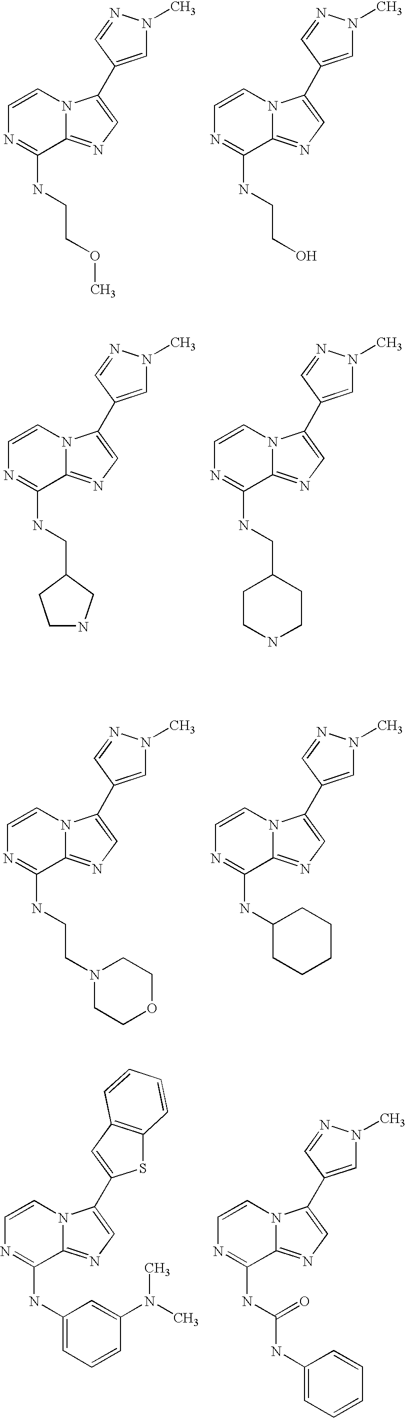

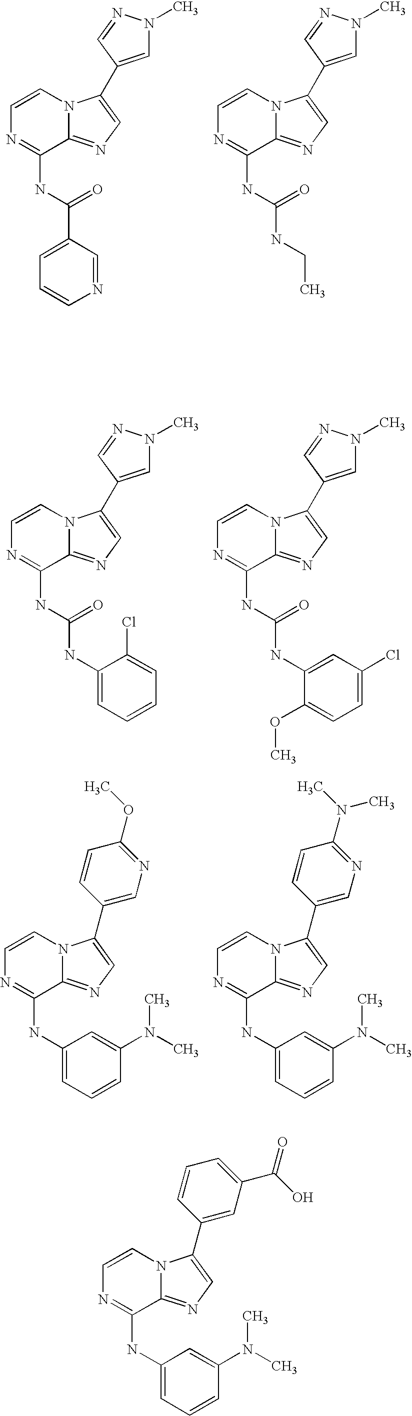

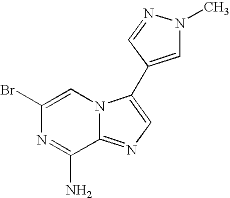

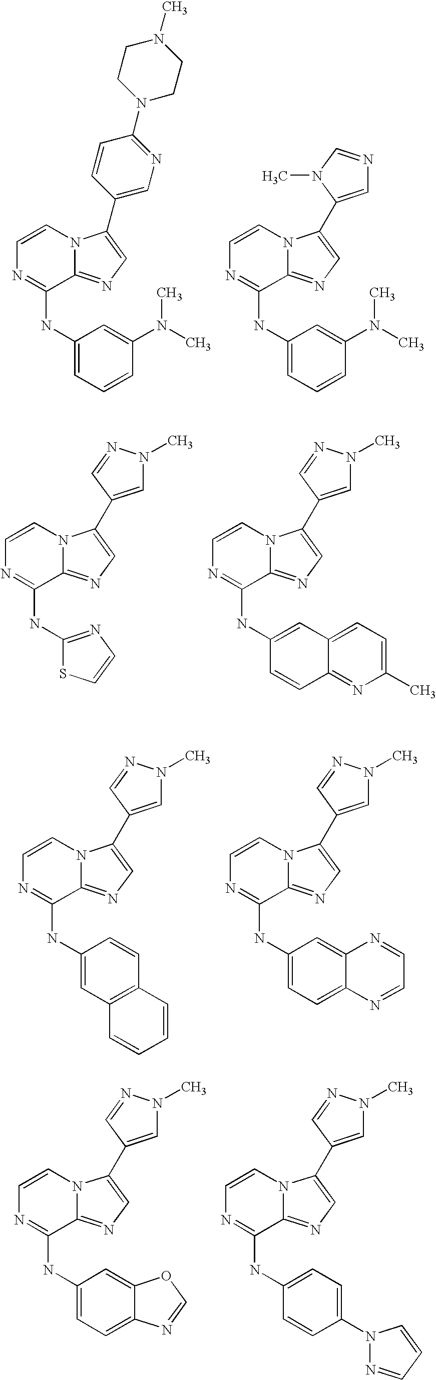

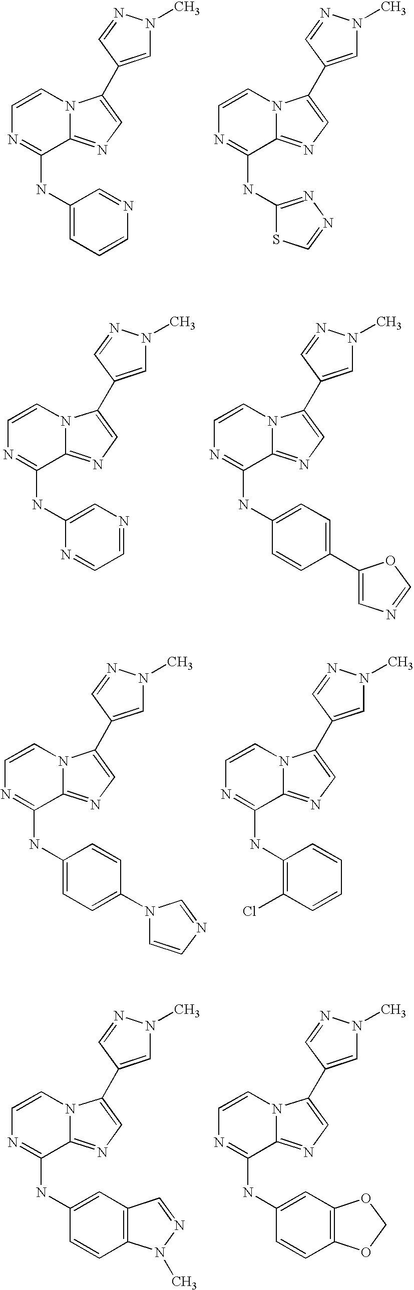

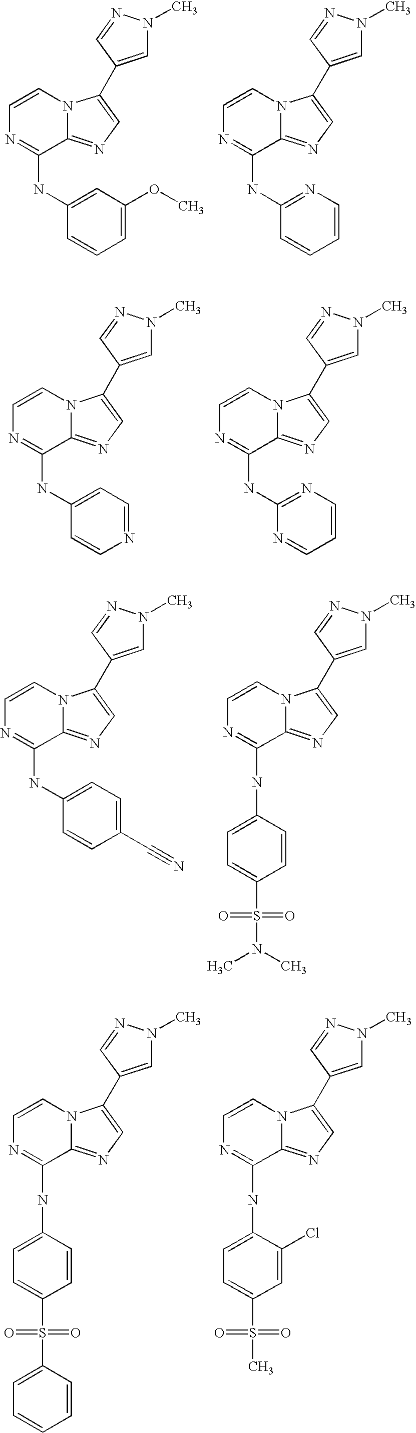

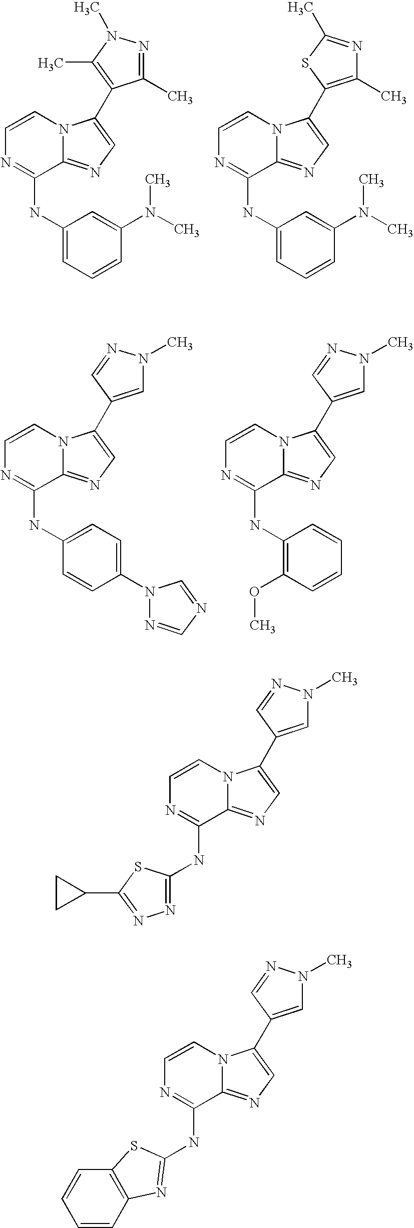

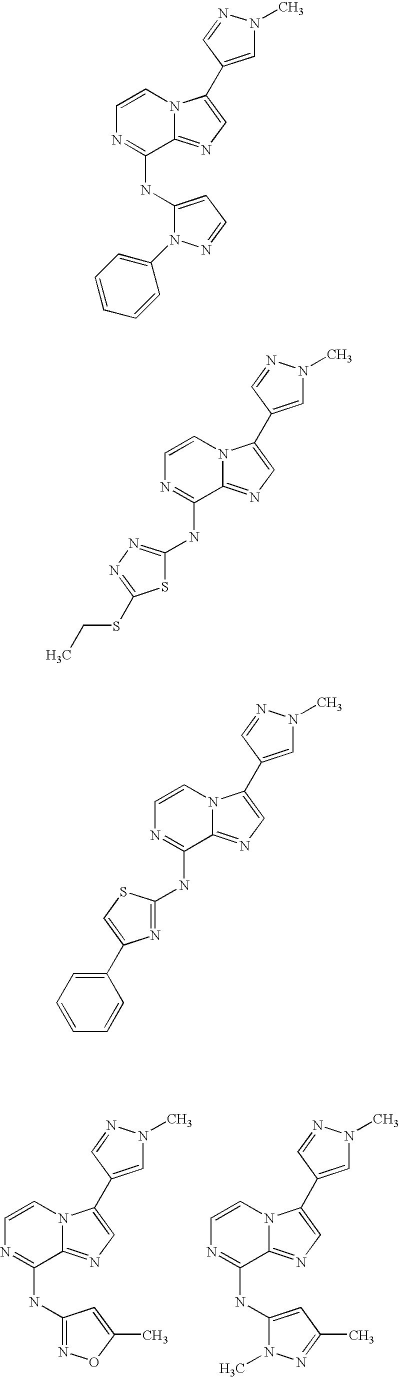

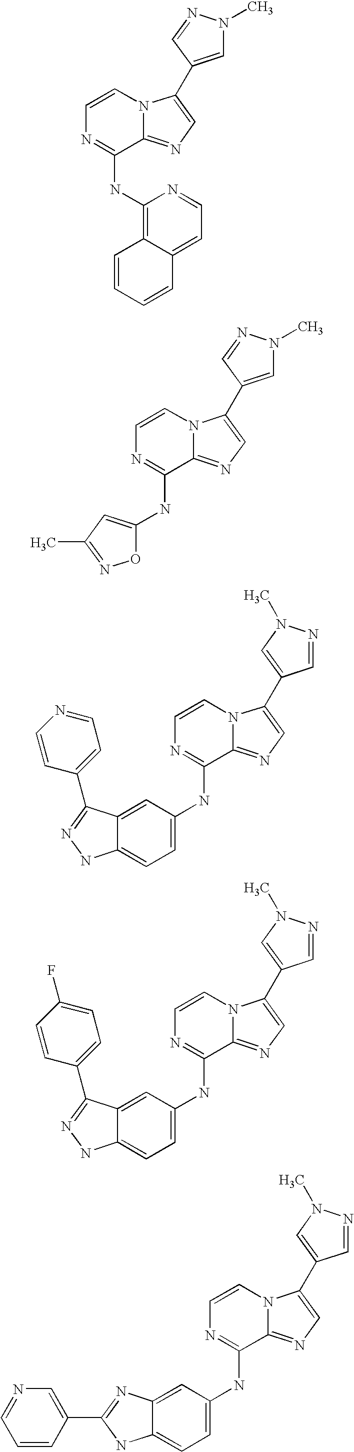

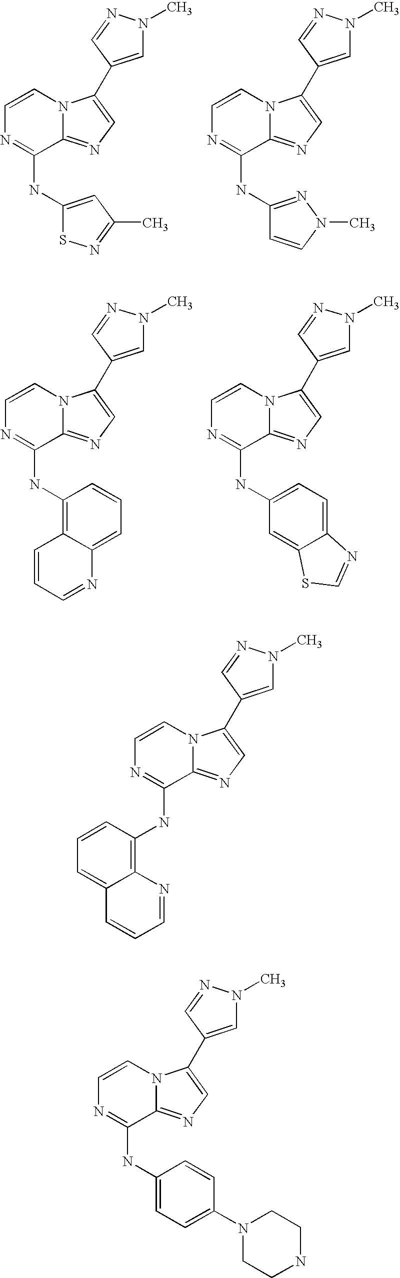

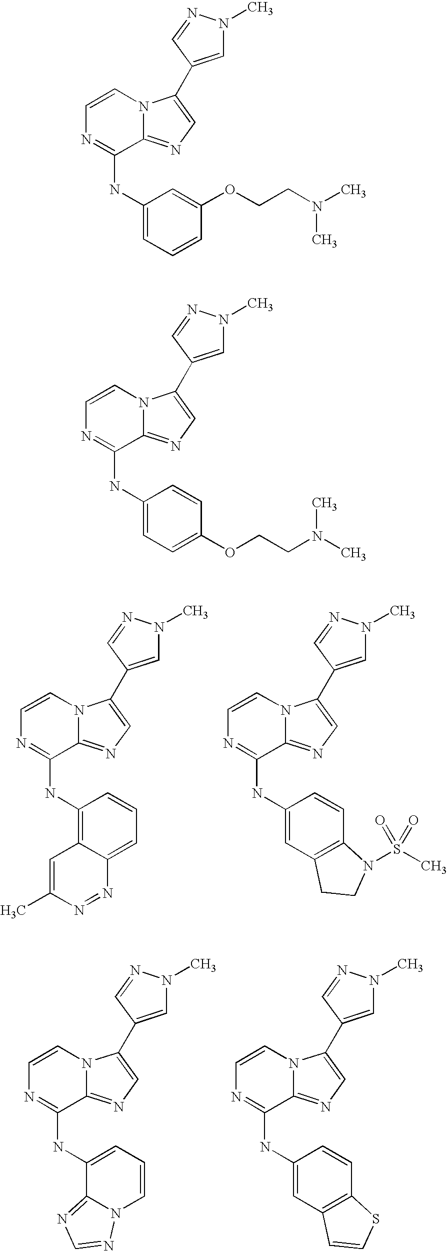

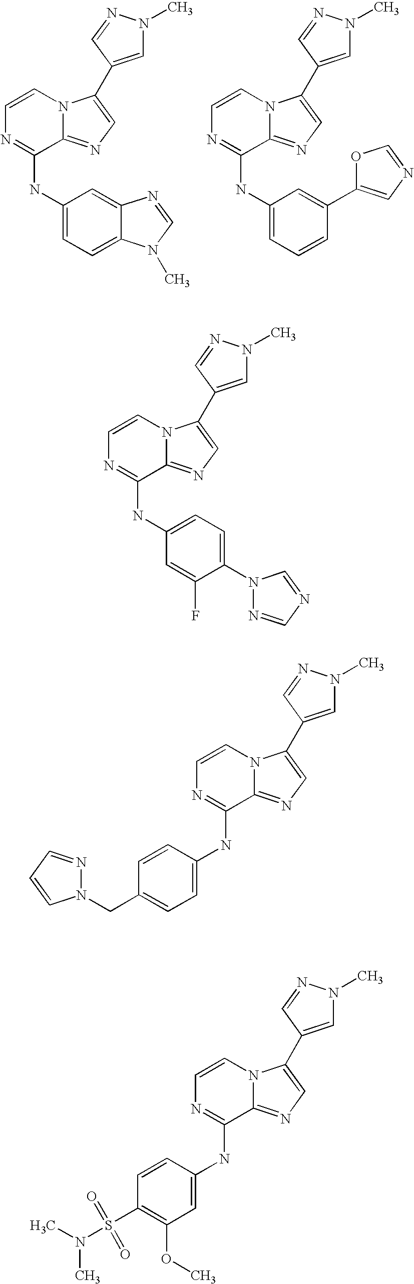

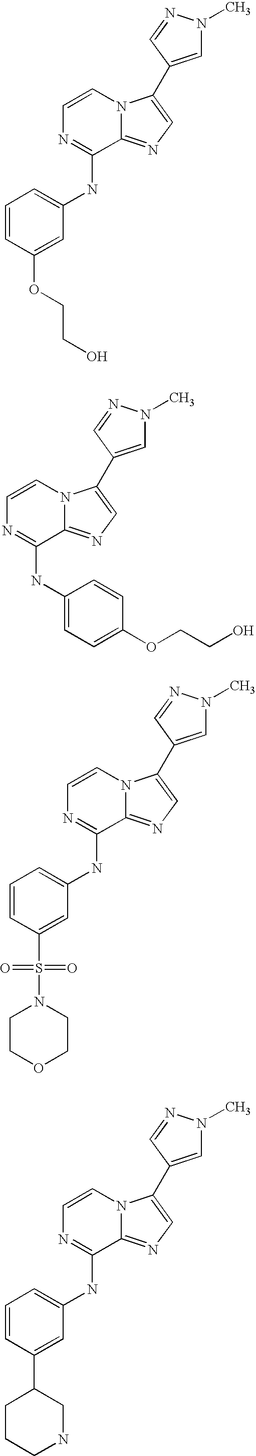

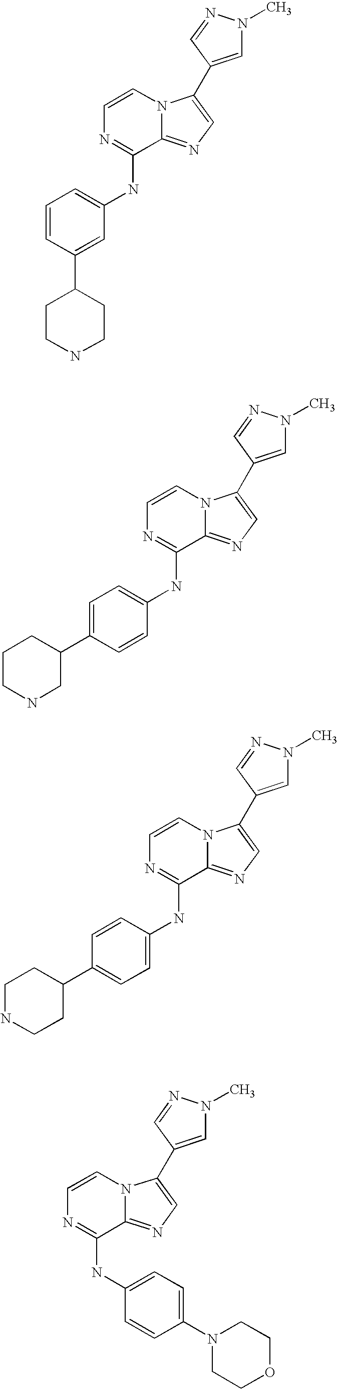

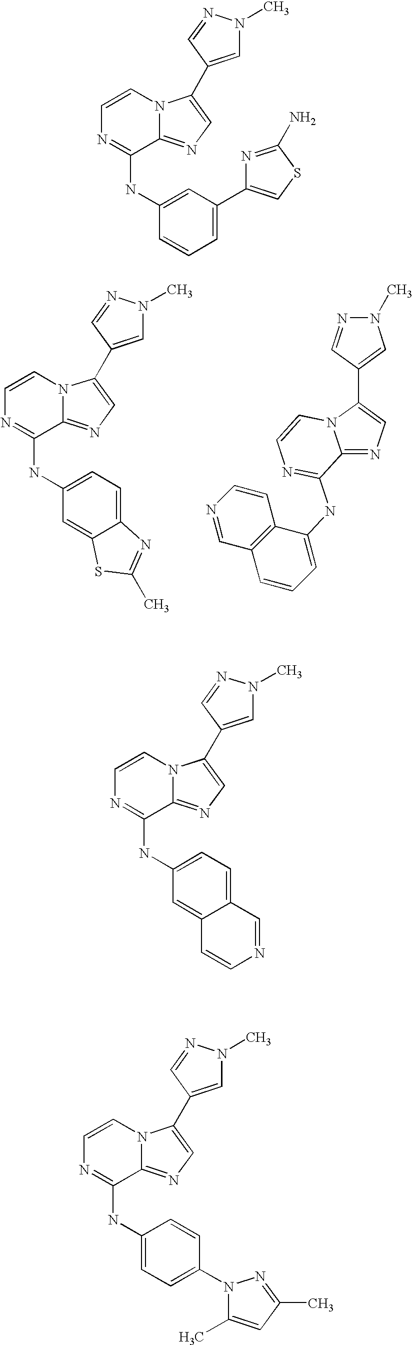

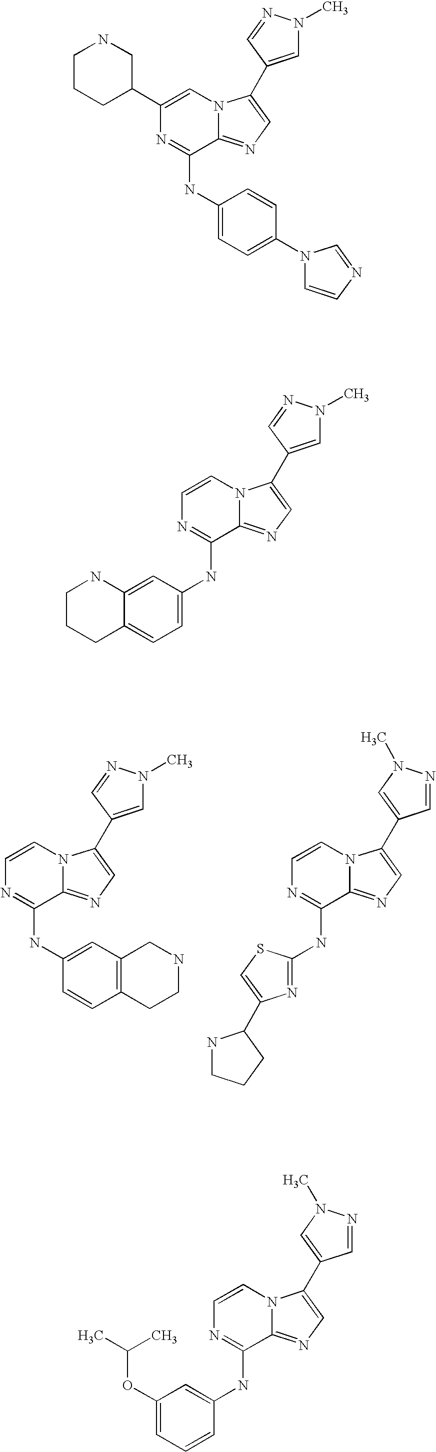

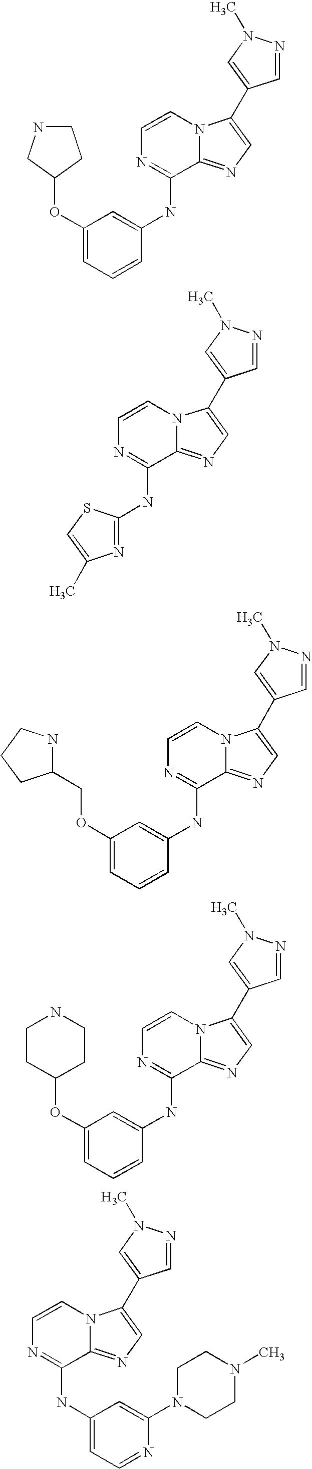

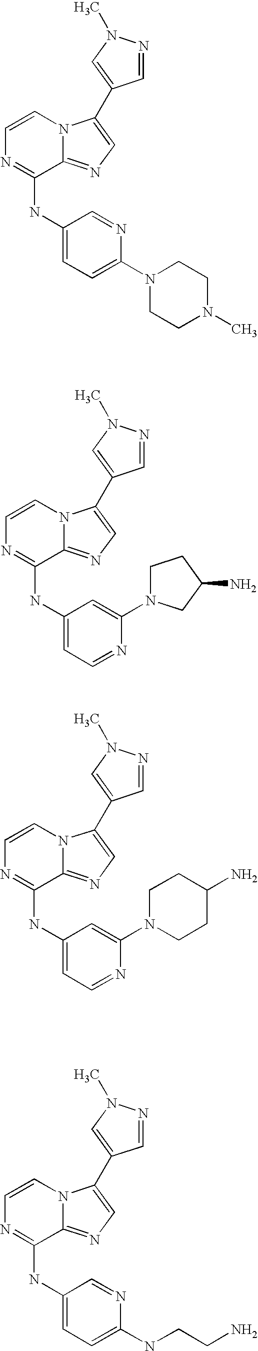

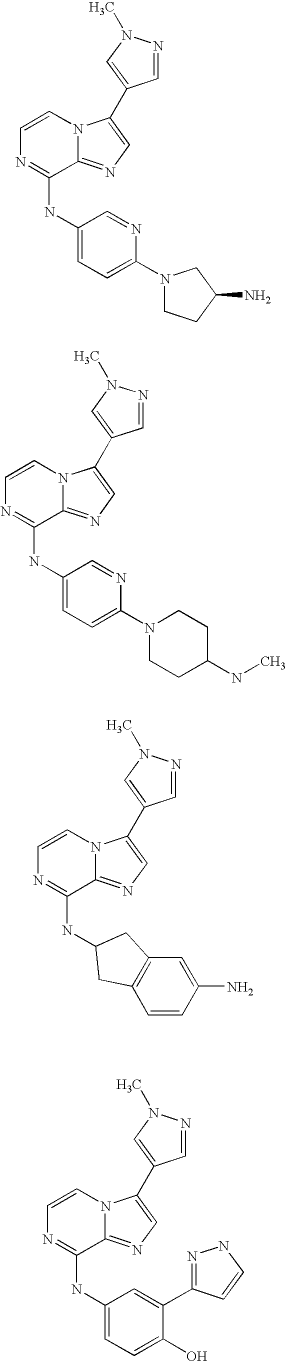

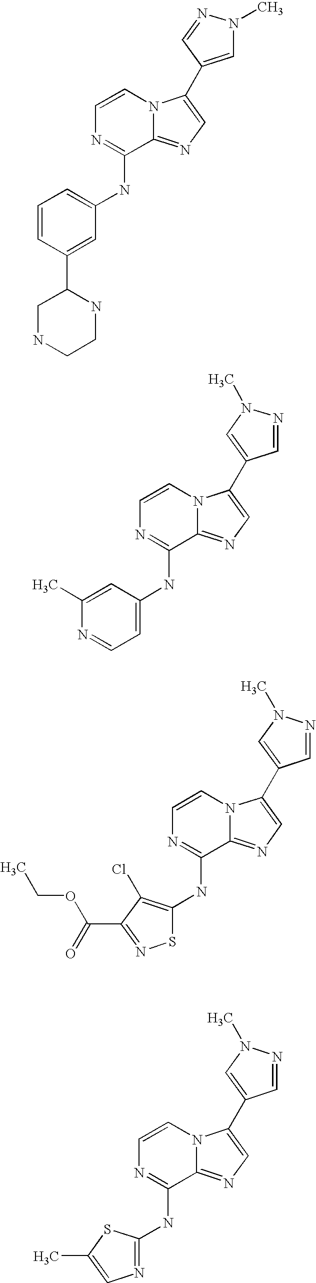

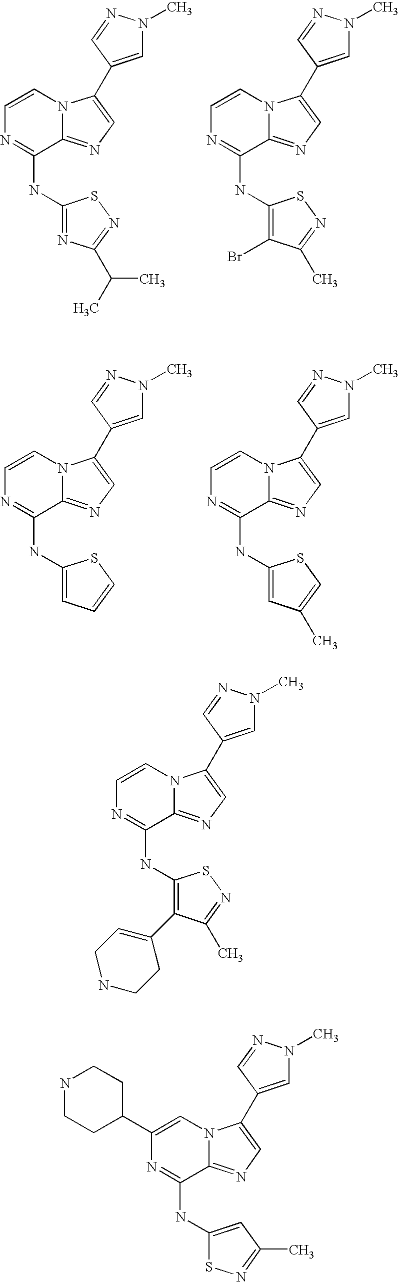

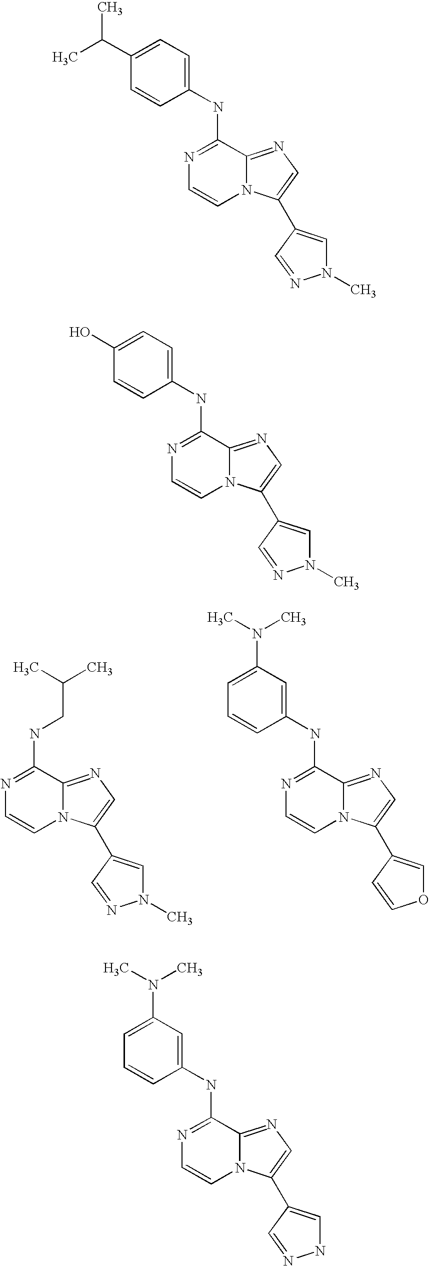

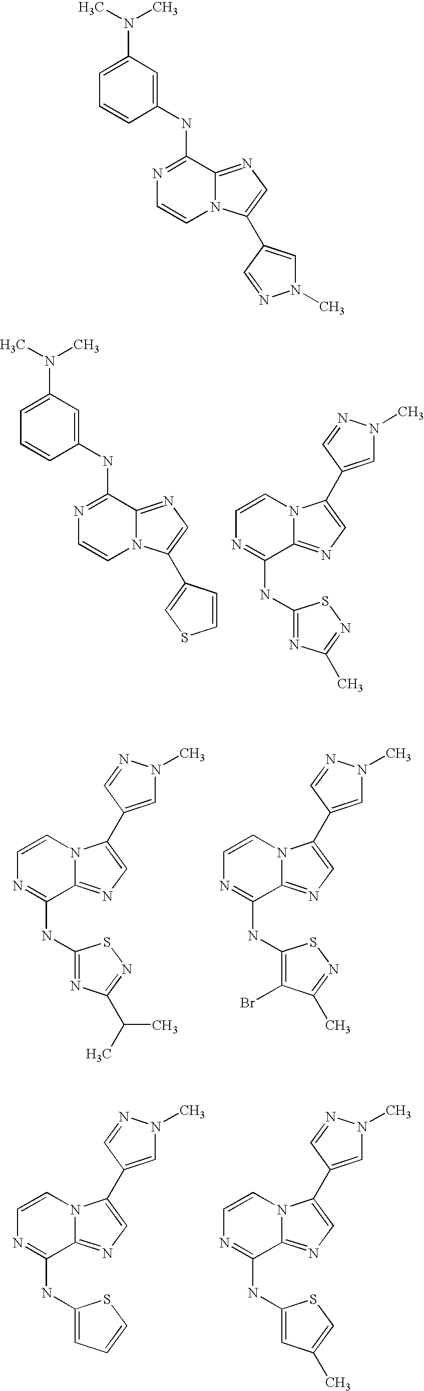

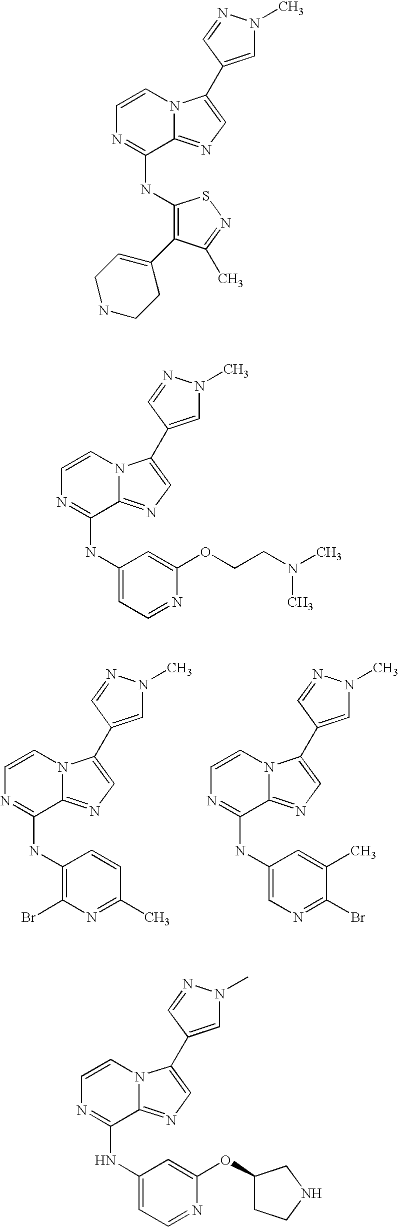

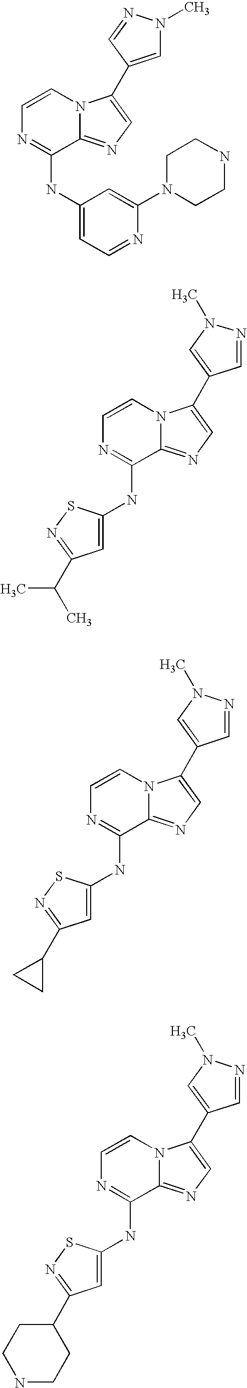

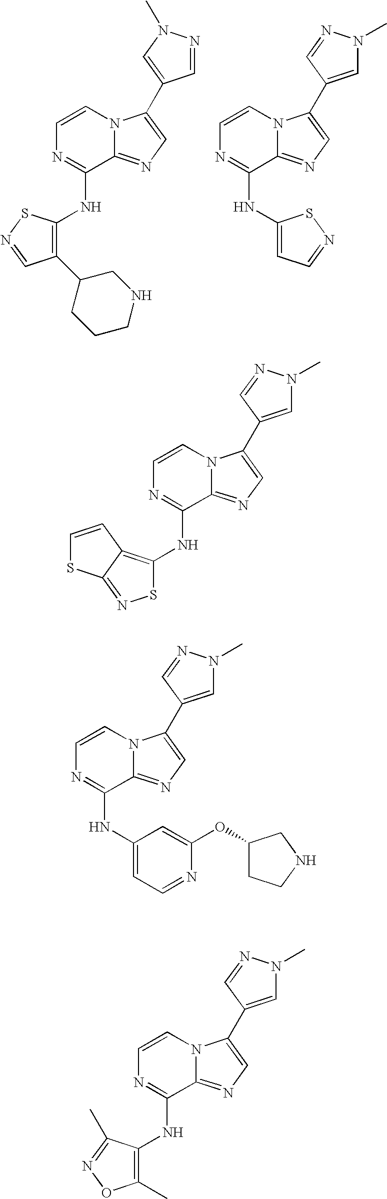

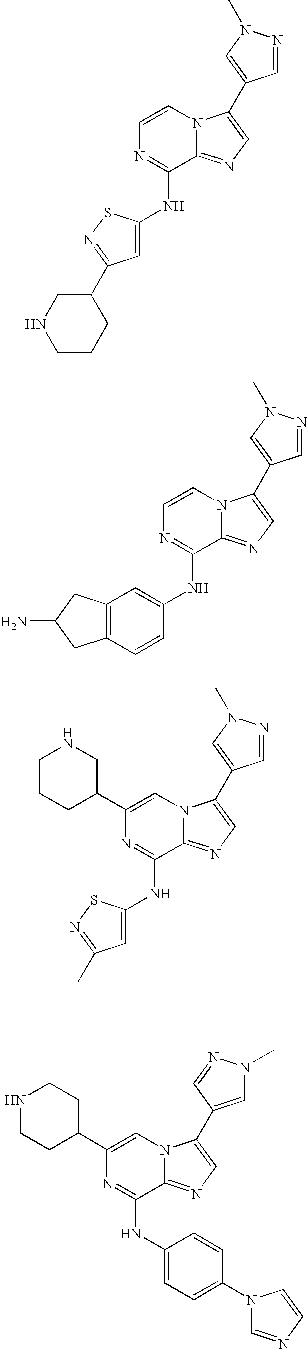

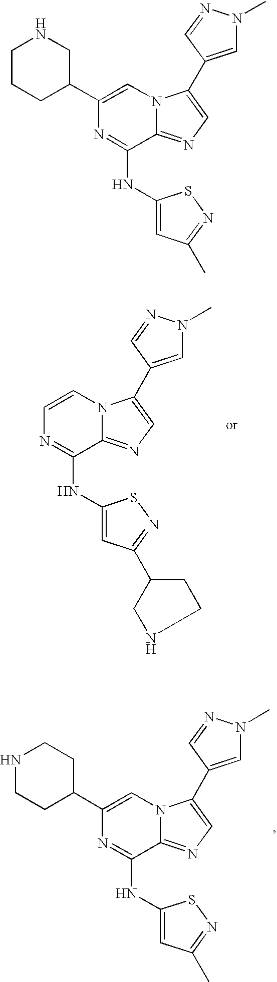

or a pharmaceutically acceptable salt, solvate, ester or prodrug thereof,
(see, WO2007/058873, filed Nov. 8, 2006);

h. A compound of the formula:

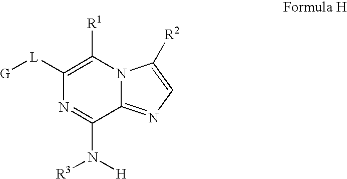

or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷,

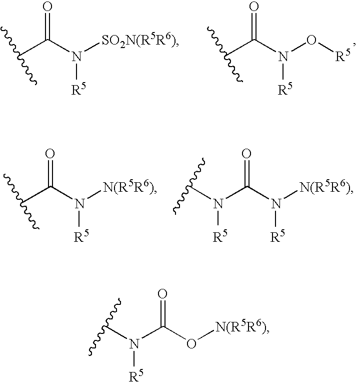

alkyl substituted with 1-6 R⁹ groups which groups can be the same or different with each R⁹ being independently selected,

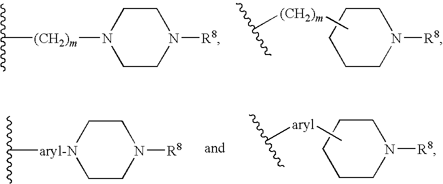

wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,

### —N(R⁵)C(O)R⁷ and —N(R⁵)C(O)NR⁵R⁶;

R³ is selected from the group consisting of H, alkyl, aryl, heteroaryl, heterocyclyl, aralkyl, heteroarylalkyl, cycloalkylalkyl, heterocyclylalkyl,

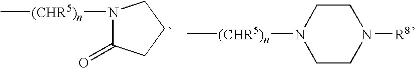

—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₘ—NR⁸, and

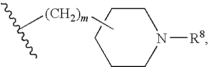

wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,

—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,

—C(O₂)R⁶, —C(O)NR⁵R⁶, —OR⁶, —C(O)R⁷, —SR⁶, —S(O₂)R⁷, —S(O₂)NR⁵R⁶,

—N(R⁵)S(O₂)R⁷, —N(R⁵)C(O)R⁷ and —N(R⁵)C(O)NR⁵R⁶;

m is 0 to 4; and

p is 0-3;

(see, WO 2008/054749, filed Oct. 29, 2007);

i. A compound of Formula I:

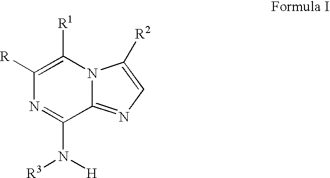

or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
   —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
   —NR⁵R⁶ (wherein the R⁵ and R⁶, together with the N of said
   —NR⁵R⁶, form a heterocyclyl ring), —S(O)R⁵, —S(O₂)R⁵, —CN, —CHO, —SR^{S}, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR^{S}, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
   X is selected from the group consisting of, —NR⁵R⁶, —OR⁵, —SO—R⁵ and —SR⁵,
   R⁷ and R⁸ are each independently hydrogen or alkyl;
   R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl, alkylN(alkyl)₂, alkylNH(alkyl), alkylN(alkenyl)2, -alkylN(alkoxyl)₂, -alkyl-SH and hydroxyalkyl, wherein each of said aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalky;
   R⁶ is selected from the group consisting of hydrogen, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl, wherein each of said aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkylSH, alkoxyl, —S-alkyl, hydroxyalkyl, and aminoalkyl;
   further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cycloalkenylalkyl, cycloalkylalkyl, heteroarylalkyl, heterocyclenylalkyl, heterocycloalkylalkyl, heteroaryl, aryl, cycloalkenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, and heterocyclenyl;
      (see, WO 2008/057512, filed Nov. 6, 2007 and Serial No. PCT US2008/007295, filed Jun. 11, 2008);

j. A compound having the formula:

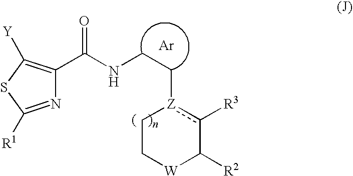

or a pharmaceutically acceptable salt, solvate, ester, prodrug or stereoisomer thereof, wherein the dashed lines indicate an optional and additional bond, and wherein:

R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;

R² is —H, -alkyl, —NH₂ or —CH₂NH₂;

R³ is —H, -alkyl, —NH₂ or —CH₂NH₂;

each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;

R⁵ is —H, -alkyl, -aryl, -heteroaryl, —NHOH;

R⁶ is —H, -alkyl or —CF₃;

R⁷ is —H, —OH, —C₁-C₆ alkyl, —O—(C₁-C₆ alkyl), or —CF₃;

R⁸ is —H, alkyl, aryl, heterocyclyl, heteroaryl or cycloalkyl;

Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)(R⁸)₂, —NHC(O)R⁸, —CF₃, —CN or NO₂, and such that when Ar is tetrahydronaphthylene, R¹ and R² cannot both be hydrogen

W is —NH— or C(R⁴)₂—, wherein both R⁴ groups and the carbon atom to which they are attached can combine to form a five to seven membered heterocyclyl or heteroaryl group;

Y is —H, -halo, -alkyl or —CN;

Z is —CR⁷— or —N—, when the optional additional bond is absent, and —C— when the optional additional bond is present;

n is an integer ranging from 0 to 2.

Compounds represented in the present application by paragraph (j) are disclosed in WO 2008/054749, filed Oct. 29, 2007.
(see, WO 2008/054749, filed Oct. 29, 2007); and

(k) A compound of Formula K:

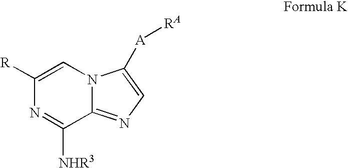

or a pharmaceutically acceptable salt, solvate, ester, prodrug or stereoisomer thereof, wherein:

R is H, halo or alkyl;

R³ is heteroaryl-X, wherein X is heterocyclylalkyl- wherein said heterocyclyl can be unsubstituted or optionally substituted with 1-4 alkyl moieties;

A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;

R^{A} is —(CH₂)₁₋₄-heteroaryl,

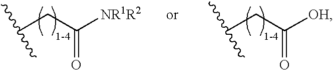

wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
   R¹ and R² together with the N to which each is attached, form a heterocyclic group selected from the group consisting of

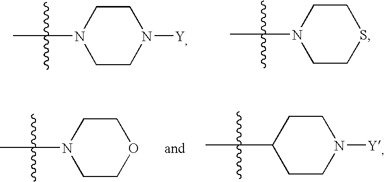

wherein
Y is alkoxyalkyl, hydroxyalkyl, dialkylaminoalkyl or alkyl, further wherein Y′ is hydroxyl.
   Compounds represented in the present application by Formula k are disclosed in U.S. Appln. No. 61/024,010, filed Jan. 28, 2008.

In another embodiment, the KSP inhibitor useful in the practice of this invention is represented by Formula AI:

AI. A compound represented by the structural Formula AI:

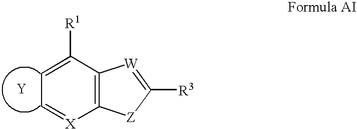

or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
   R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
   each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
   each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
   each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
   or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
   each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
   each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
   each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰) NR¹⁰R¹¹, and —NR¹⁰)C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰C(O)OR¹¹, and —NR¹⁰)C(O)R⁴⁰;
   or two R⁸ groups, when attached to the same carbon atom, are optionally taken together with the carbon atom to which they are attached to form a C═O or a C═S group;
   each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
   each R¹⁰) is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
   each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
   each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₅—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
   R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
   with the proviso that the compound of Formula AI excludes any one of the following:

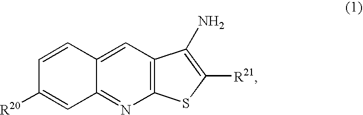

wherein R²⁰ is H, —CH₃ or —OCH₃ and R²¹ is —C(O)CH₃, —C(O)CH═CH-phenyl or —C(O)CH═CH-(4-methoxyphenyl);

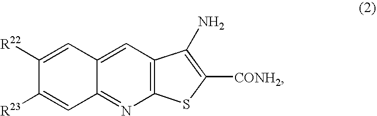

wherein R²² and R²³ are independently H or methoxy;

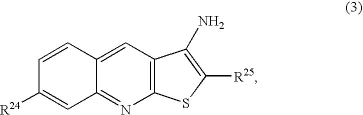

wherein R²⁴ is methyl, methoxy or —Cl and R²⁵ is —CONH₂ or —CO₂Et;

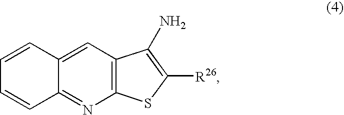

wherein R²⁶ is —CO₂Me, —CO₂Et, —CO₂H, —C(O)-phenyl, —C(O)-p-methylphenyl, —C(O)-p-bromophenyl, —C(O)CH₃, —CN, —C(O)NH-phenyl, —C(O)NH-p-methoxyphenyl, —C(O)NHNH₂, —C(O)NH-p-chlorophenyl,

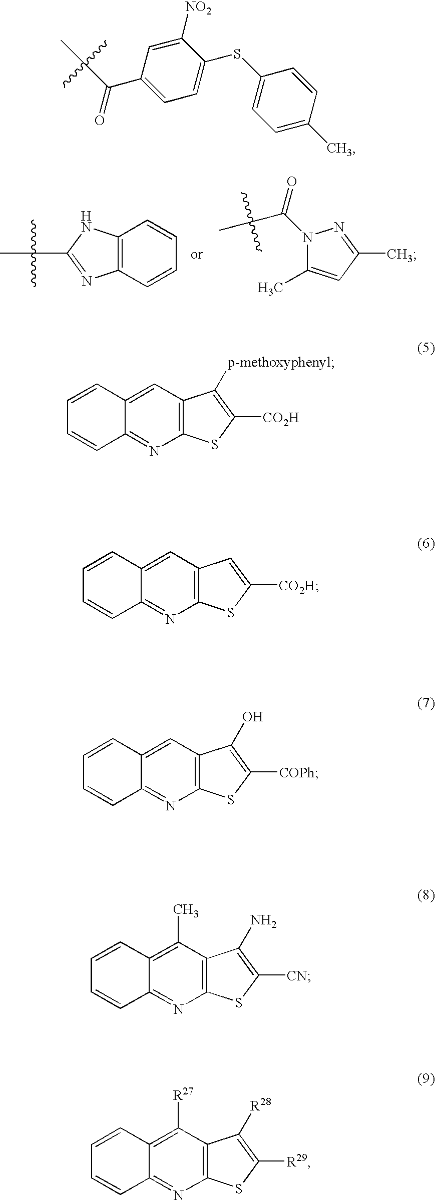

wherein:
R²⁷ is H, —OH, —OCH₃ or —OCH(CH₃)₂,
R²⁸ is —OH, —OCH₂CN or —OC(O)NH(CH₂)₅CN, and
R²⁹ is —C(O)OCH(CH₃)₂ or —C(O)O-cyclohexyl;

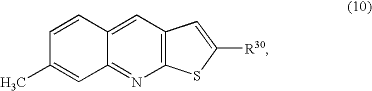

wherein:
R³⁰ is

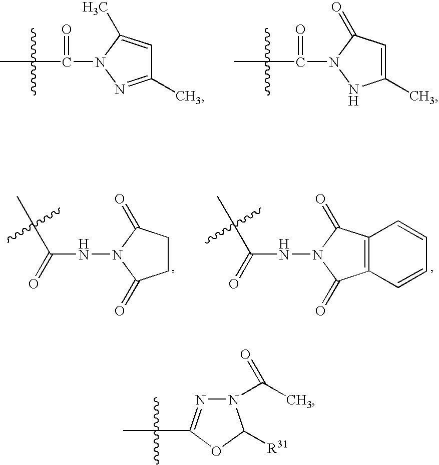

—CO₂CH₃, —CO₂C₂H₅, —C(O)NH₂, —C(O)NHNH₂, or —C(O)NHCH₃ and R³¹ is C₆H₅, p-OHC₆H₄ or p-CH₃C₆H₄;

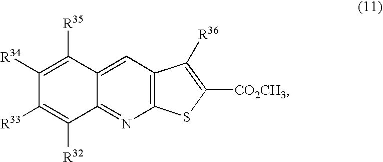

wherein:
R³² is H or NO₂,
R³³ and R³⁴ are independently H, —OCH₃ or —OC₂H₅,
R³⁵ is H or —OCH₃, and
R³⁶ is H, CH₃ or C₆H₅;

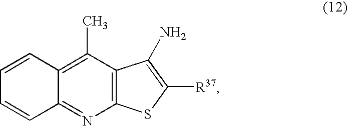

wherein:
R³⁷ is —CO₂Me, —CO₂Et, —CO₂H, —C(O)NH₂, —C(O)NHNH₂, —CN, —C(O)NH-p-methoxyphenyl, —C(O)NH-(2-pyridyl) or

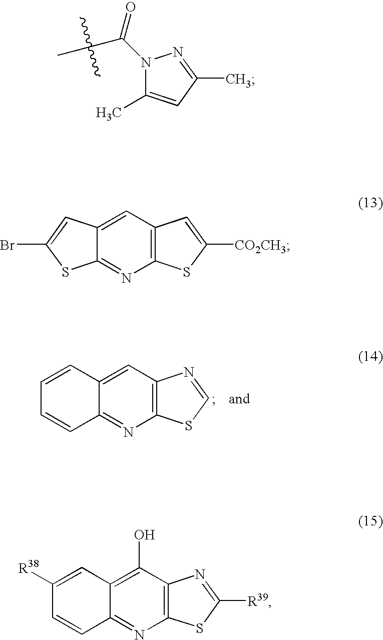

wherein R³⁸ is H, methyl or CF₃ and R³⁹ is SMe, SOMe, SO₂Me, Cl, NH(CH₂)NEt₂, or N—(N′-methyl)piperazinyl.
   Compounds represented in the present application by Formula AI are disclosed in WO 2006/098962, filed Mar. 7, 2006.

In another embodiment, the KSP inhibitor useful in the practice of this invention is represented by Formula BI:

BI. A compound represented by the structural Formula BI:

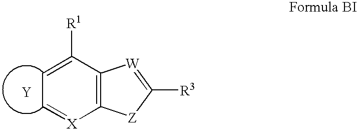

or a pharmaceutically acceptable salt, solvate or ester thereof, wherein:

ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;

W is N or C(R¹²)

X is N or N-oxide;

Z is S, S(═O) or S(═O)₂;

R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;

each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵,—C(S)NR⁷NR⁴R⁵, —C(S)NR⁴OR⁷, —C(O)SR⁷, —NR⁴R⁵, —NR⁴C(O)R⁵, —NR⁴C(S)R⁵, NR⁴C(S)R⁵, —NR⁴C(O)OR⁷, —NR⁴C(S)OR⁷, —OC(O)NR⁴R⁵, OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;

or two R² moieties on the same carbon atom are optionally taken together with the carbon atom to which they are attached to form a C═O, a C═S or an ethylenedioxy group;

R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵,

### —C(O)N(R⁷)(CR⁴⁰R⁴¹)₁₋₅—OR⁷, —C(S)NR⁷(CH₂)₁₋₅NR⁴R⁵, and

—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;

each of R⁴ and R⁵ is independently selected from the group consisting of H, alkyl, cycloalkyl, cycloalkenyl, heterocyclyl, heterocyclenyl, aryl, heteroaryl, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;

or R⁴ and R⁵, when attached to the same nitrogen atom, are optionally taken together with the nitrogen atom to which they are attached to form a 3-6 membered heterocyclic ring having 0-2 additional heteroatoms selected from N, O or S;

each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;

each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;

each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —C(O)R¹⁰R¹¹, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring; or two R⁸ groups, when attached to the same carbon, are optionally taken together with the carbon atom to which they are attached to form a C═O or a C═S group;

each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;

each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;

each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,

—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀NR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;

R⁴⁰ and R⁴¹ can be the same or different, each being independently selected from the group consisting of H, alkyl, aryl, heteroaryl, heterocyclyl, heterocyclenyl, cycloalkyl and cycloalkenyl;

each R⁴² is independently selected from the group consisting of halo, alkyl, cycloalkyl, heterocyclyl, aryl, heteroaryl, —NO₂, —(C₁-C₆ alkyl)-OR¹⁰,

### —CN, —NR¹⁰R¹¹, —C(O)R¹⁰, —C(O)OR¹⁰, —C(O)NR¹⁰R¹¹, —CF₃, —OCF₃,

### —N(R¹⁰)C(O)R¹¹, and —NR¹⁰C(O)OR¹¹;

with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;

with the further proviso that the compound of Formula (31) is other than any of the following compounds (1)-(9) shown immediately below:

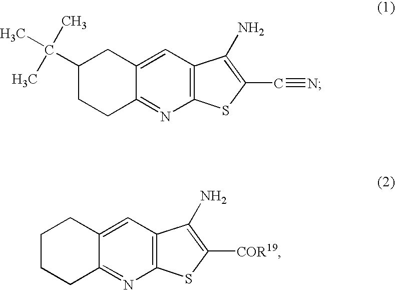

wherein:

### R¹⁹ is —NHOH, —OMe, —OEt, —O-n-propyl, or —O-i-propyl;

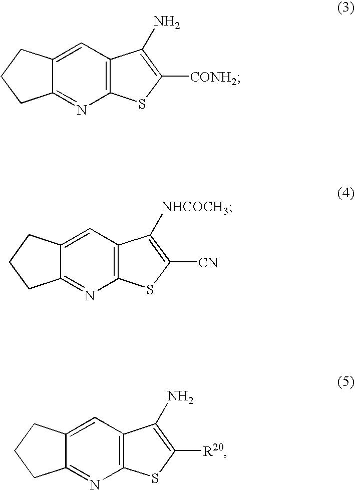

wherein:

R²⁰ is —CN, —C(O)C₆H₅, —CO₂C₂H₅, —CO₂H, or —C(O)NH₂;

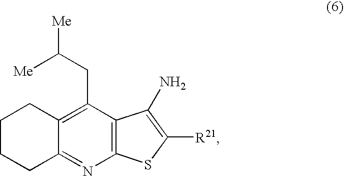

wherein:

R²¹ is 4-ClC₆H₄C(O)— or 4-PhC₆H₄C(O)—;

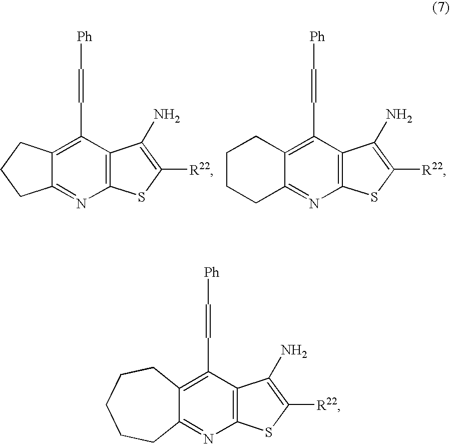

wherein:

R²² is —CN, —C(O)CH₃ or —CO₂C₂H₅;

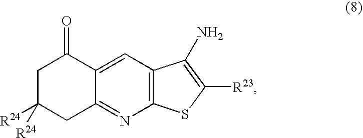

wherein:

R²³ is —C(O)NH₂, —C(O)NHPh, or benzoyl and R²⁴ is H or methyl;

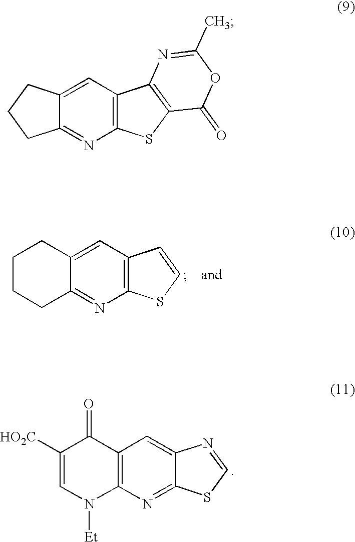

Compounds represented in the present application by Formula BI are disclosed in WO2006/098961, filed Mar. 7, 2006.

In another embodiment, the KSP inhibitor useful in the practice of this invention is represented by Formula DI:

DI: A compound of Formula DI

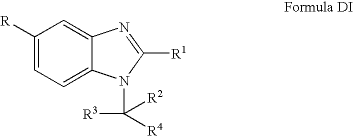

or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(O)H(alkyl), —C(═O)NH(cycloalkyl), —C(O)N(alkyl)₂, —C(═O)OH, —C(═O)Oalkyl, —C(═O)heterocyclyl, —C(═O)NH(aryl), wherein when each of said cycloalkyl, aryl, heterocyclyl, hetetoaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of allyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(O)₂NH(alkyl), S(═O)₂NH(cycloalkyl), —S(═O)₂N(alkyl)₂, —S(═O)₂heterocyclyl, —S(O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
with the proviso that:
(1) when R is —C(═O)Oalkyl, R¹ is NH(aryl) wherein said aryl is optionally substituted, R² and R³ are both H, and R⁴ is phenyl, then said R⁴ phenyl is substituted with at least one haloalkyl group;
(2) when R is —C(═O)Oalkyl, R¹ is NH(aryl) or NH(alkyl), wherein the “alkyl” and “aryl” portions of said R¹ are independently optionally substituted, R² and R³ are both H, and R⁴ is optionally substituted heteroaryl, then said R⁴ heteroaryl is other than thiophenyl and furanyl;
(3) when R is —C(═O)Oalkyl wherein said alkyl is optionally substituted, and R¹ is NH(aryl) wherein said aryl is unsubstituted or substituted with at least one substituent selected from the group consisting of halo, nitro, and alkyl, then R⁴ is other than unsubstituted cycloalkyl;
(4) when R is —C(═O)Oalkyl wherein said alkyl is optionally substituted, R¹ is —NH₂, and —CR²R³ is absent, then R⁴ is other than optionally substituted cycloalkyl;
(5) when R is —C(═O)NH₂, then R¹ is —NH(alkyl) or —N(alkyl)(aryl), wherein each of said alkyl and aryl are independently optionally substituted;
(6) when R is —COOH or —C(═O)Oalkyl, wherein said alkyl is optionally substituted, R¹ is —NH(alkyl), or —N(alkyl)₂ wherein the “alkyl” portion of each of said R¹ is optionally substituted, and —CR²R³ is absent, then R⁴ is other than unsubstituted cycloalkyl;
(7) when R is —C(═O)Oalkyl wherein said alkyl is optionally substituted, R¹ is —NH₂, NH(alkyl), or NH(aryl), wherein the “alkyl” and “aryl” portion each of said R¹ is optionally independently substituted, then R⁴ is other than unsubstituted alkyl or alkyl substituted with at least one moiety selected from the group consisting of alkoxy, —N(alkyl)₂, heterocyclyl, and heteroaryl;
(8) when R is —C(═O)Oalkyl wherein said alkyl is optionally substituted, R¹ is —NH₂, and —C(R²)(R³)— is absent, then R⁴ is other than optionally substituted cycloalkyl, cycloalkenyl, heterocyclenyl, or heteroaryl;
(9) when R is —C(═O)Oalkyl, wherein said alkyl is optionally substituted, R¹ is —NH(heterocyclyl) wherein said heterocyclyl is optionally substituted, R² and R³ are both H, then R⁴ is other than optionally substituted heteroaryl;
(10) when R is —C(═O)Oalkyl, wherein said alkyl is optionally substituted, R² and R³ are both H, and R⁴ is optionally substituted aryl or heteroaryl, then R¹ is other than —NH₂ or —NH(alkyl) wherein said alkyl is optionally substituted;
(11) when R is —C(═O)OH, —C(R²)(R³)— is absent, and R⁴ is optionally substituted cycloalkyl, then R¹ is other than —NHC(═O)alkyl, —NH(aryl), or —N(alkyl)(aryl) wherein the “alkyl” and “aryl” portions of said R¹ are optionally independently substituted;
(12) when R is —C(═O)OH, R¹ is —NH₂, and R² and R³ are both H, then R⁴ is other than optionally substituted aryl;
(13) when R is —C(═O)Oalkyl wherein said alkyl is optionally substituted, R¹ is optionally substituted heterocyclyl or heteroaryl, R² and R³ are both H, and R⁴ is aryl, then said R⁴ aryl is substituted with at least one haloalkyl group;
(14) when R is —C(O)OH, R¹ is optionally substituted heterocyclyl, R² and R³ are both H, and R⁴ is aryl, then said R⁴ aryl is substituted with at least one haloalkyl group; or
(15) when R is H or halo, and R⁴ is aryl, then said R⁴ aryl is substituted with at least one haloalkyl group,
(16) when R is H, R² and R³ are both H, R¹ is —NH(aryl), and R⁴ is aryl substituted with at least one haloalkyl group, then said “aryl” of R¹ is substituted with group(S) other than halo or haloalkyl;
(17) when R is —C(═O)NH(aryl) or —C(═O)N(alkyl)(aryl), then R¹ is other than optionally substituted heterocyclyl; or
(18) when R is H, R¹ is other than —NH₂ or —NH(heterocyclyl.
   Compounds represented in the present application by Formula DI are disclosed in PCT U.S. 2008/007295, filed Jun. 11, 2008.

In another embodiment, the present invention discloses a method of treating or ameliorating cancer comprising administering sequentially to a mammal in need of such treatment an amount of at least one first compound, wherein said first compound is an anti-mitotic agent selected from the group consisting of Taxanes, paclitaxel, docetaxel, Cenp-E Inhibitor (such as for example GSK-923295), Abraxane, Epothilone, Monastrol, and KSP inhibitors such as Ispinesib SB-715992 (Cytokinetics), and compounds of Formulas A-D as described above; followed by administering at least one second compound, wherein said second compound is an aurora kinase inhibitor selected from the group consisting of compounds represented by Formulas E-K as described above.

In another embodiment, the present invention discloses a method of inducing endoreduplication in cancer cells comprising pretreatment of cancer cells with at least one anti-mitotic agent, wherein said anti-mitotic agent is selected from the group consisting of Taxanes, paclitaxel, docetaxel, Cenp-E Inhibitor (such as for example GSK-923295), Abraxane, Epothilone, Monastrol, and KSP inhibitors such as Ispinesib SB-715992 (Cytokinetics) and compounds represented by Formulas A-D as described above; with subsequent exposure to aurora kinase inhibitor for about 1-12 hours, for example about 4 hours, wherein said aurora kinase inhibitor is selected from the group of compounds represented by Formulas E-K as described above.

In another embodiment, the present invention results in endoreduplication of the cancer cells, which results in death of the cancer cells, which include (but is not limited to) the following:

tumor of the bladder, breast (including BRCA-mutated breast cancer), colorectal, colon, kidney, liver, lung (including small cell lung cancer and non-small cell lung cancer), head and neck, esophagus, bladder, gall bladder, ovary, pancreas, stomach, cervix, thyroid, prostate, and skin, including squamous cell carcinoma;

leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma and Burkett's lymphoma;

chronic lymphocytic leukemia (“CLL”),

acute and chronic myelogenous leukemia, myelodysplastic syndrome and promyelocytic leukemia;

fibrosarcoma, rhabdomyosarcoma;

mantle cell lymphoma, myeloma;

astrocytoma, neuroblastoma, glioblastoma, malignant glial tumors, hepatocellular carcinoma, gastrointestinal stromal tumors (“GIST”) and schwannomas;

melanoma, multiple myeloma, seminoma, teratocarcinoma, osteosarcoma, xenoderoma pigmentosum, keratoctanthoma, thyroid follicular cancer and Kaposi's sarcoma.

Furthermore, the present invention results in endoreduplication of the cancer cells, which include more preferably, colon cancer cells, breast cancer cells, lung cancer cells, prostrate cancer cells and ovarian cancer cells.

In another embodiment, administration of the combination treatment of at least one anti-mitotic agent followed by at least one of the aurora kinase inhibitors of the present invention can be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The combination treatment of the present invention may also be useful in inhibiting tumor angiogenesis and metastasis.

In another embodiment, cancer cells are pretreated with at least one anti-mitotic agent preferably for about 1 to 72 hours.

In another embodiment, cancer cells are pretreated with at least one anti-mitotic agent preferably for about 12 to 72 hours.

In another embodiment, cancer cells are pretreated with at least one anti-mitotic agent more preferably, for about 12 to 24 hours.

In another embodiment, cancer cells are pretreated with at least one anti-mitotic agent most preferably for about 16 hours.

In another embodiment, cancer cells are pretreated with at least one anti-mitotic agent most preferably for about 4 hours.

In another embodiment, cancer cells are pretreated with at least one anti-mitotic agent most preferably for about 2 hours.

In another embodiment, cancer cells are pretreated with at least one anti-mitotic agent most preferably for about 1 hour.

In another embodiment, cancer cells are exposed to said aurora kinase inhibitor for about 1-72 hours after the antimotic agent has been administered.

In another embodiment, cancer cells are exposed to said aurora kinase inhibitor for about 2-24 hours after the antimotic agent has been administered.

In another embodiment, cancer cells are exposed to said aurora kinase inhibitor preferably for about 1-12 hours after the antimotic agent has been administered.

In another embodiment, cancer cells are exposed to said aurora kinase inhibitor more preferably, for about 1-6 hours after the antimotic agent has been administered.

In another embodiment, cancer cells are exposed to said aurora kinase inhibitor more preferably, for about 1-4 hours after the antimotic agent has been administered.

In another embodiment, cancer cells are exposed to said aurora kinase inhibitor more preferably, for about 2-4 hours after the antimotic agent has been administered.

In another embodiment, cancer cells are exposed to said aurora kinase inhibitor most preferably for about 2 hours after the antimotic agent has been administered.

In another embodiment, cancer cells are exposed to said aurora kinase inhibitor most preferably for about 4 hours after the antimotic agent has been administered.

In another embodiment, cancer cells are exposed to said aurora kinase inhibitor for about 1-4 hours after the antimotic agent has been administered to said cancer cells for about 1-16 hours.

In another embodiment, cancer cells are exposed to said aurora kinase inhibitor for about 1-4 hours after the antimotic agent has been administered to said cancer cells for about 2-16 hours.

In another embodiment, cancer cells are exposed to said aurora kinase inhibitor for about 1-4 hours after the antimotic agent has been administered to said cancer cells for about 2 hours.

In another embodiment, cancer cells are exposed to said aurora kinase inhibitor for about 1-4 hours after the antimotic agent has been administered to said cancer cells for about 4 hours.

In another embodiment, cancer cells are exposed to said aurora kinase inhibitor for about 1 hour after the antimotic agent has been administered to said cancer cells for about 1 hours.

In another embodiment, cancer cells are exposed to said aurora kinase inhibitor for about 2 hours after the antimotic agent has been administered to said cancer cells for about 2 hours.

In another embodiment, cancer cells are exposed to said aurora kinase inhibitor for about 4 hours after the antimotic agent has been administered to said cancer cells for about 4 hours.

In another embodiment, the anti-mitotic agents used in the present invention include Taxanes, paclitaxel, docetaxel, Cenp-E Inhibitor (such as for example GSK-923295), Abraxane, Epothilone, Monastrol, and KSP inhibitors such as Ispinesib SB-715992 (Cytokinetics), and compounds of Formulas A-D as described above.

In yet another embodiment, a method of retarding the growth of a tumor in vivo comprising the steps of (a) first administering to tumor in vivo at least one anti-mitotic agent selected from the group consisting of Taxanes, paclitaxel, docetaxel, Cenp-E Inhibitor (such as for example GSK-923295), Abraxane, Epothilone, Monastrol, and KSP inhibitors such as Ispinesib SB-715992 (Cytokinetics), and compounds represented by Formulas A-D as described above

and (b) subsequently administering to said tumor at least one aurora kinase inhibitor, wherein said aurora kinase inhibitor is selected from the group consisting of a compound represented by Formulas E-K as described above.

In another embodiment, the present invention provides a method of inducing polyploidy in cancer cells comprising the steps of (a) first administering to cancer cells vivo at least one anti-mitotic agent selected from the group consisting of Taxanes, paclitaxel, docetaxel, Cenp-E Inhibitor (such as for example GSK-923295), Abraxane, Epothilone, Monastrol, and KSP inhibitors such as Ispinesib SB-715992 (Cytokinetics), and compounds represented by Formulas A-D as described above and (b) subsequently administering to said cancer cells at least one aurora kinase inhibitor, wherein said aurora kinase inhibitor is selected from the group consisting of a compound represented by Formulas E-K as described above.

Another embodiment of the present invention provides a pharmaceutical composition and method of treatment wherein the compounds covered by Formulas A-D are selected from the group consisting of compounds listed in Table 1 shown immediately below:

TABLE 1

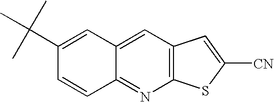

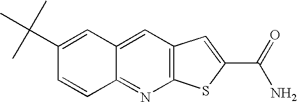

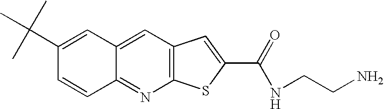

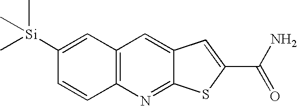

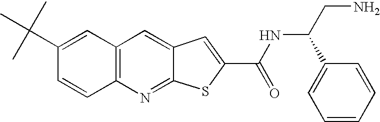

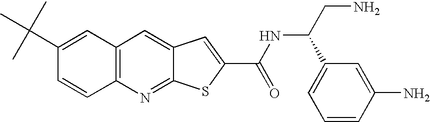

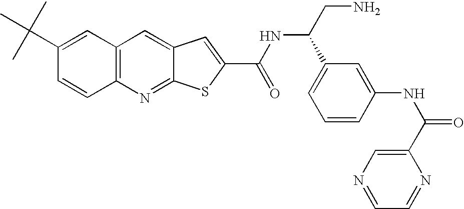

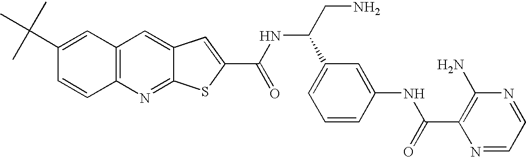

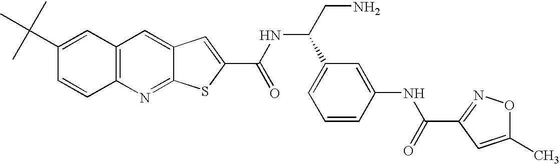

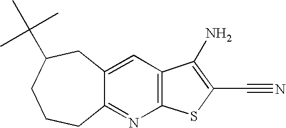

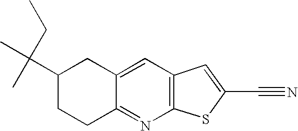

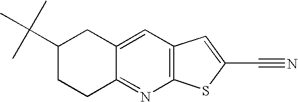

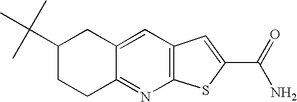

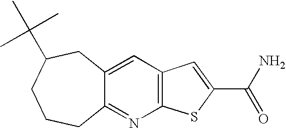

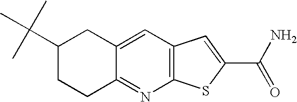

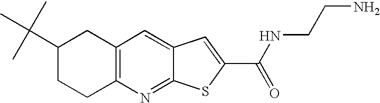

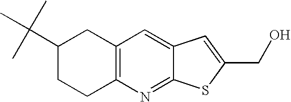

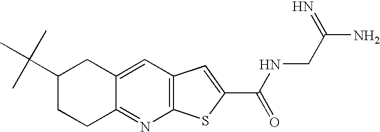

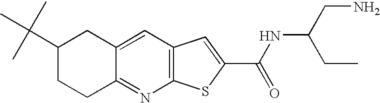

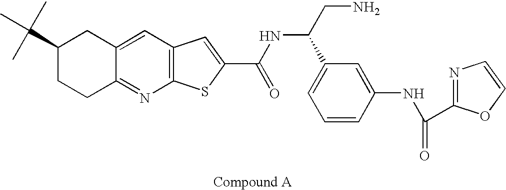

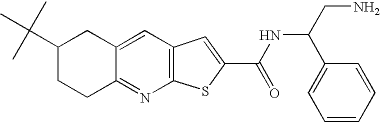

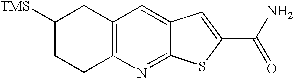

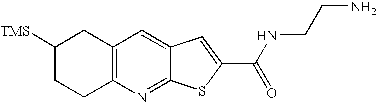

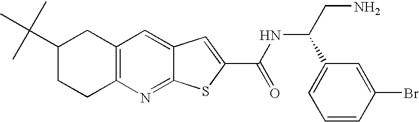

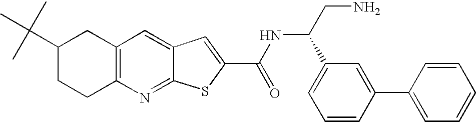

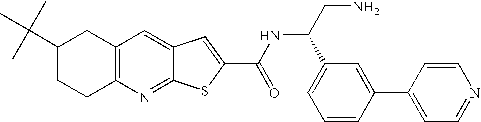

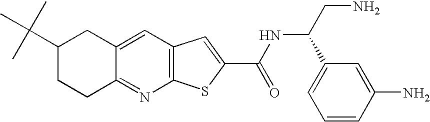

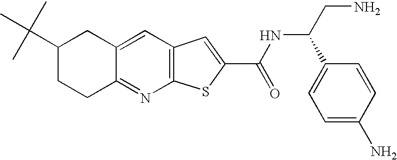

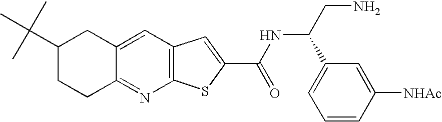

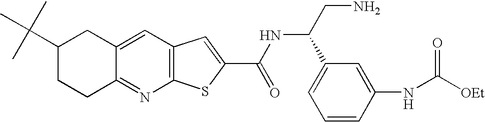

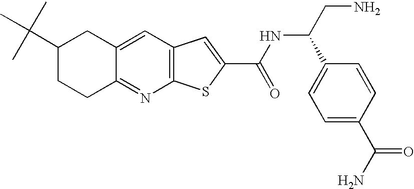

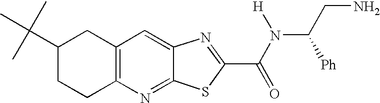

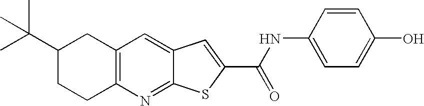

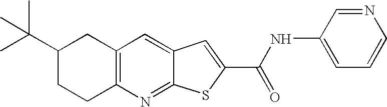

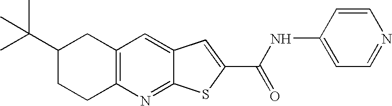

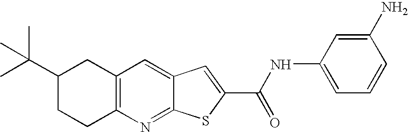

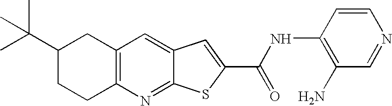

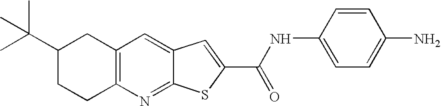

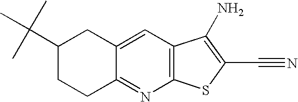

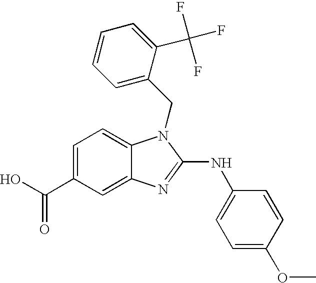

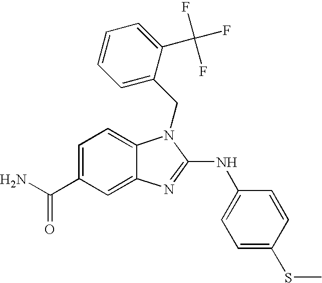

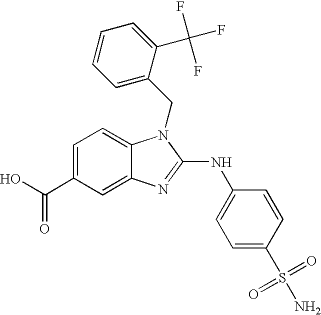

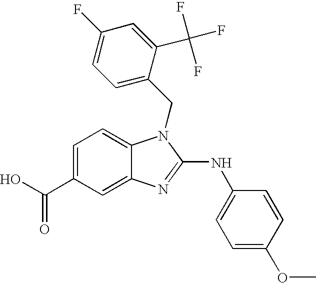

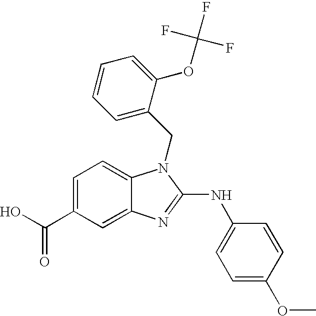

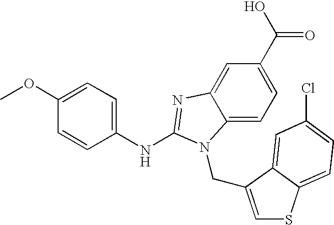

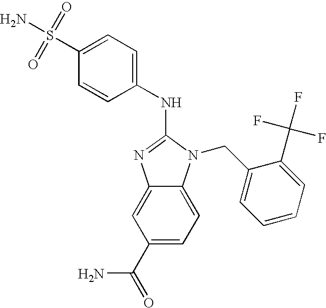

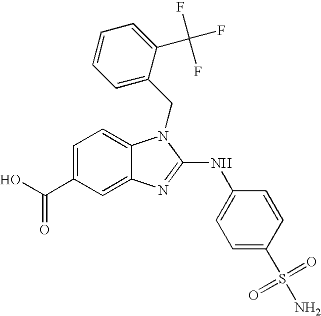

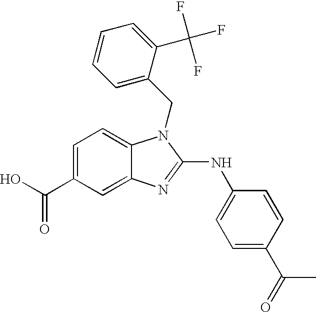

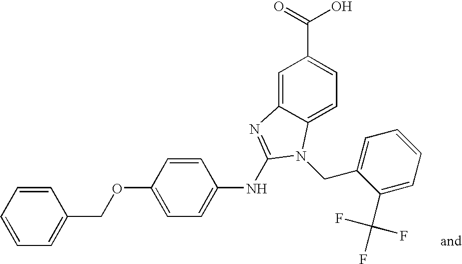

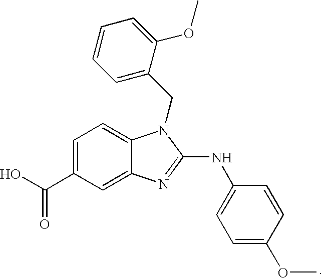

or a pharmaceutically acceptable salt or solvate thereof. The compounds of Table 1 above are disclosed in the following references: WO 2006/098962, filed Mar. 7, 2006, WO2006/098961, filed Mar. 7, 2006, U.S. Pub. No. 2006/0258699, filed Mar. 7, 2006, and U.S. Appln. No. 60/939,963, filed May 24, 2007.

In yet another embodiment, the present invention provides a pharmaceutical composition and method of treatment as described earlier wherein the compounds covered by Formulas E-K are selected by the group of compounds listed in Table 2 shown immediately below:

TABLE 2

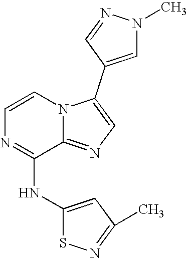

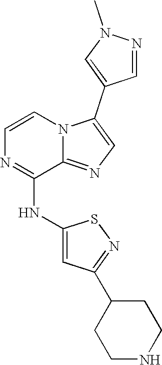

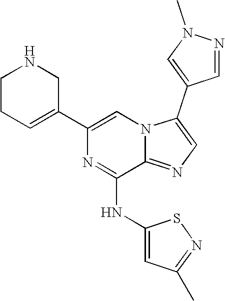

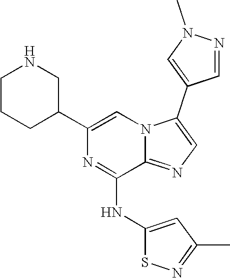

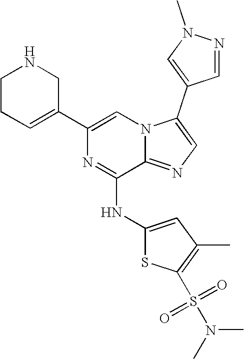

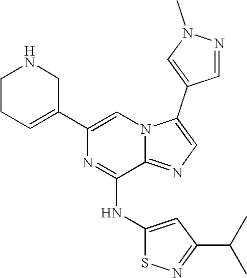

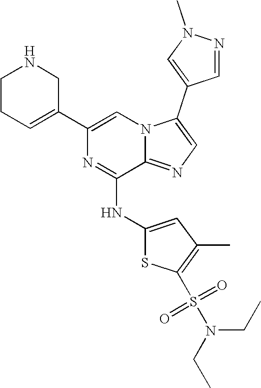

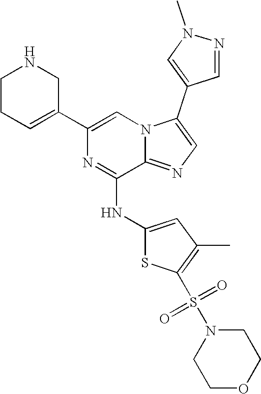

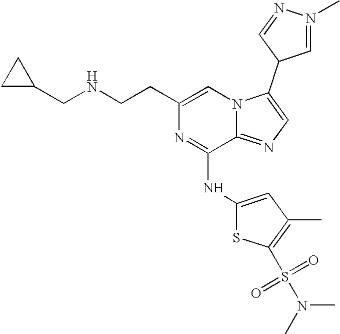

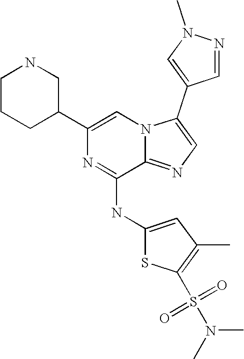

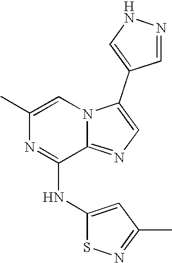

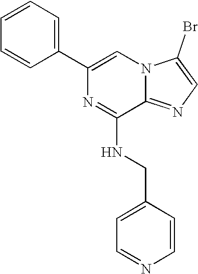

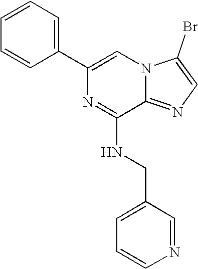

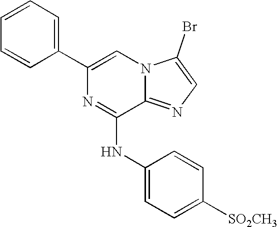

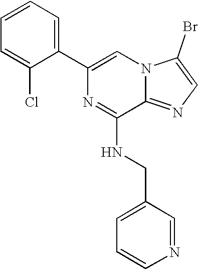

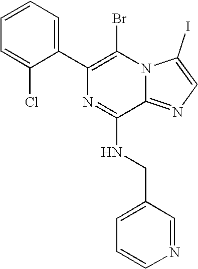

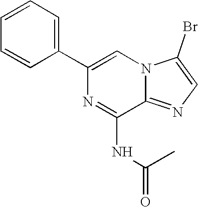

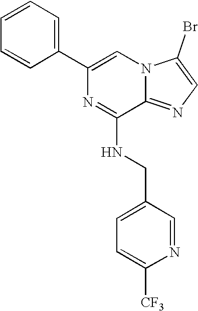

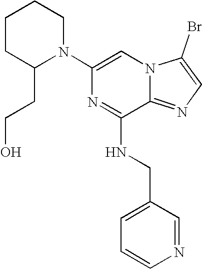

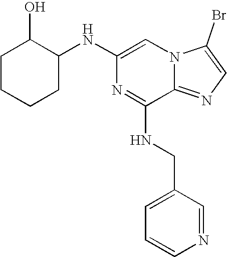

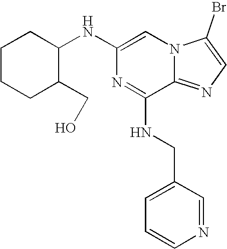

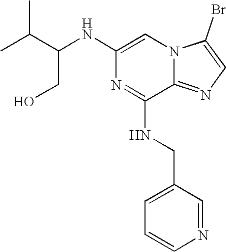

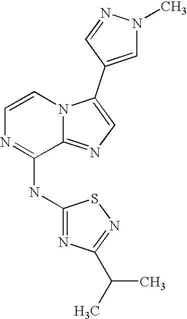

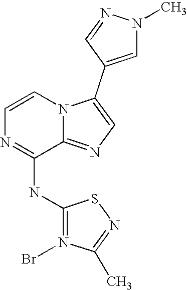

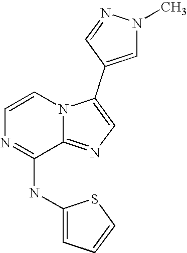

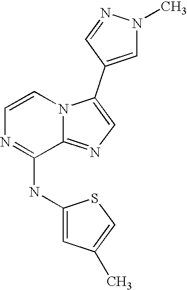

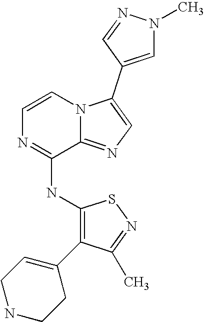

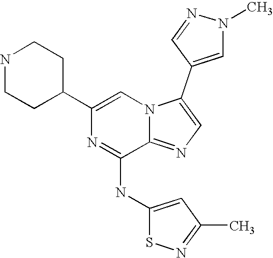

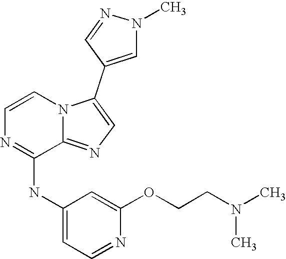

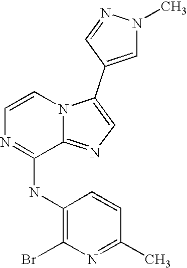

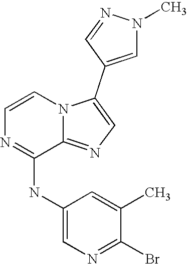

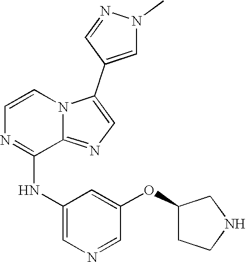

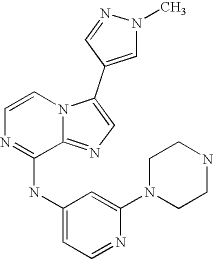

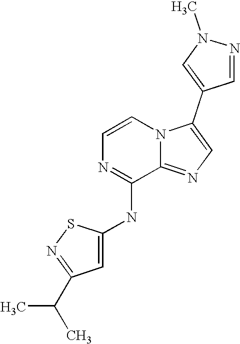

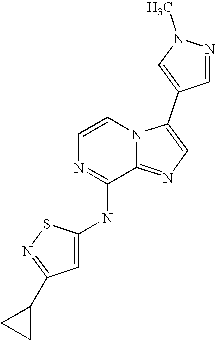

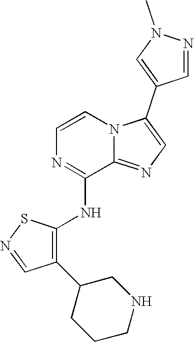

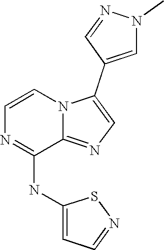

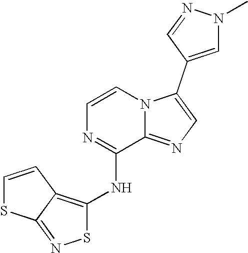

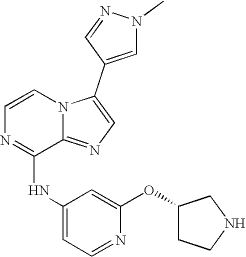

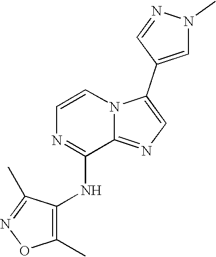

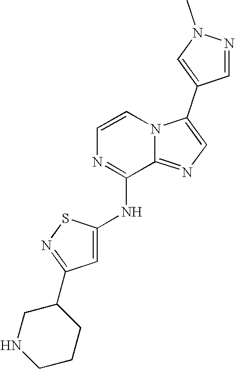

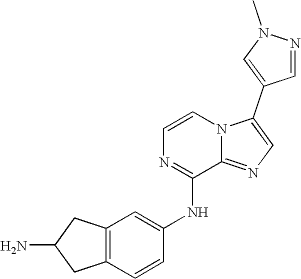

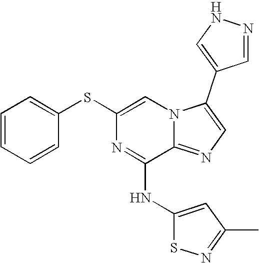

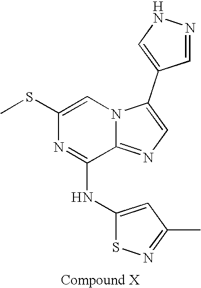

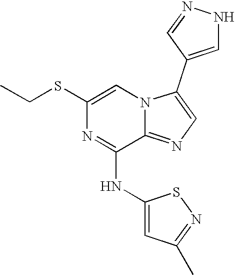

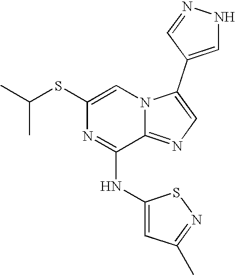

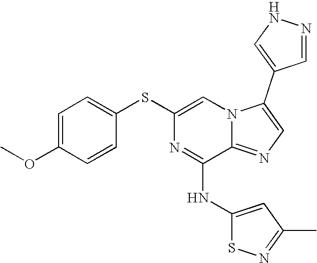

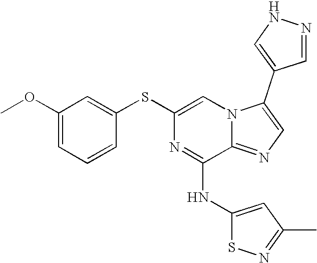

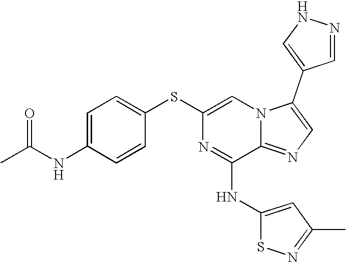

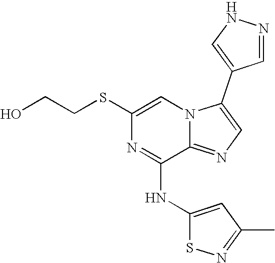

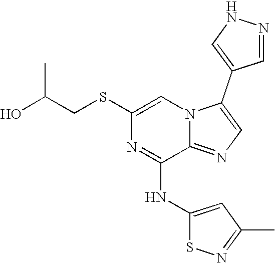

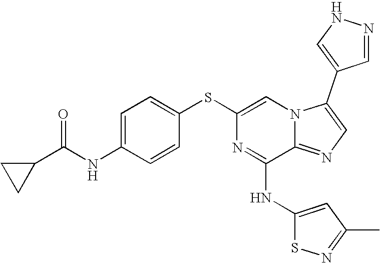

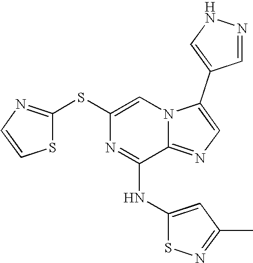

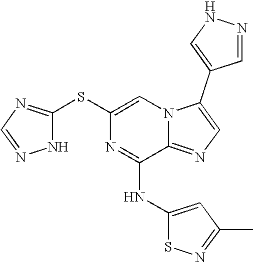

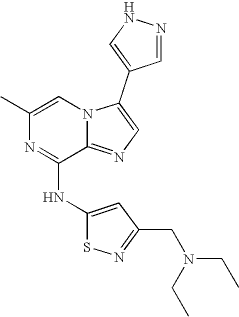

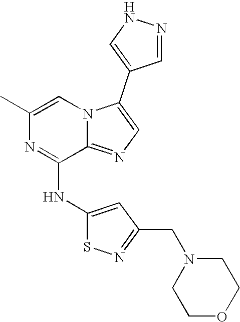

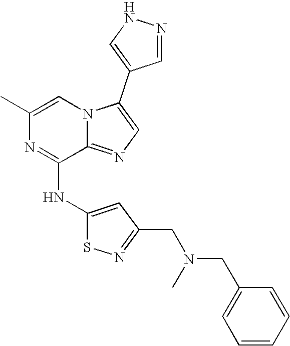

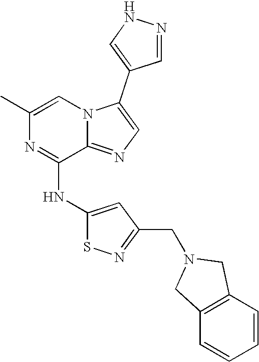

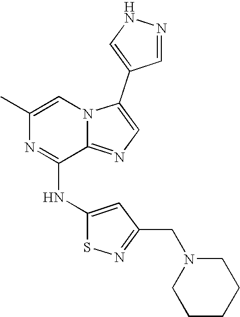

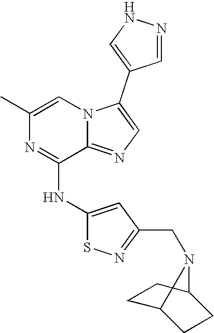

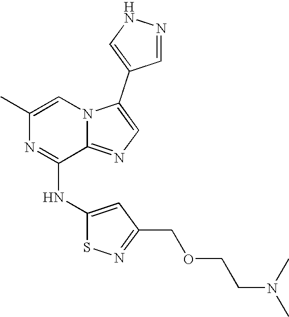

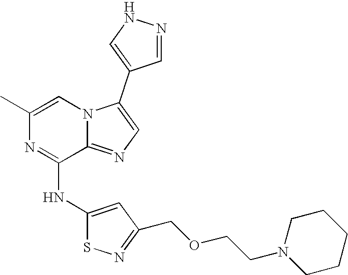

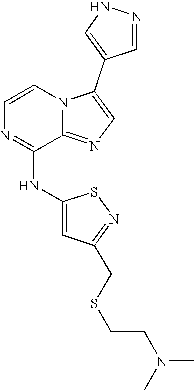

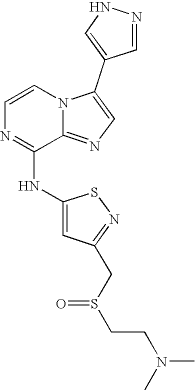

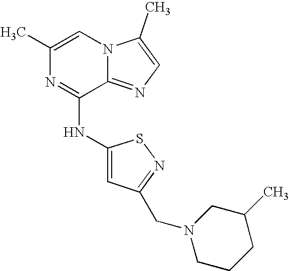

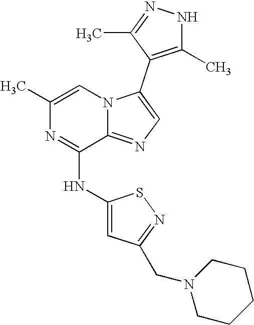

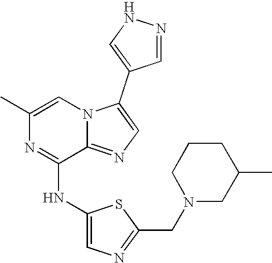

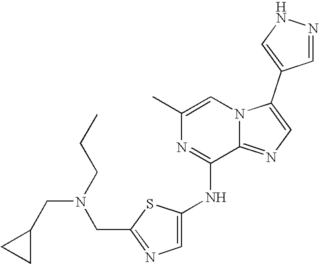

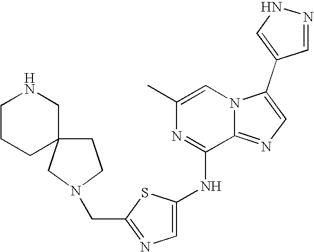

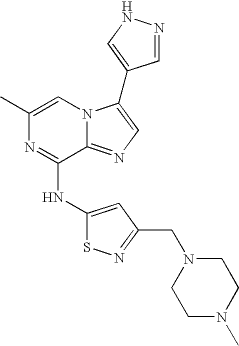

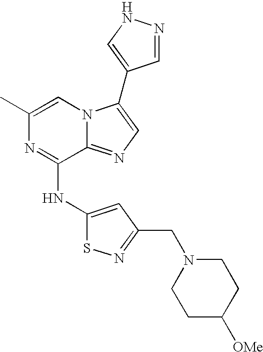

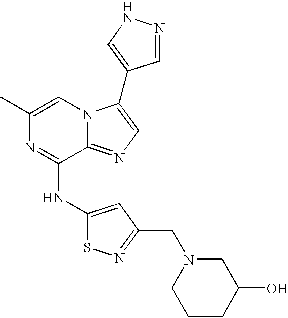

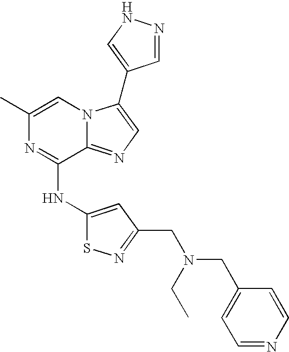

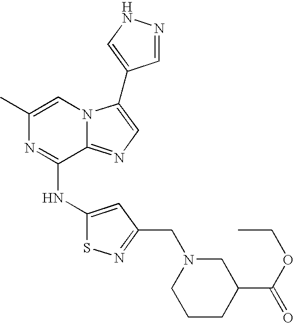

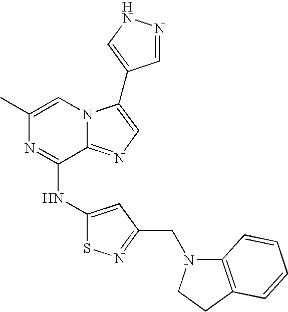

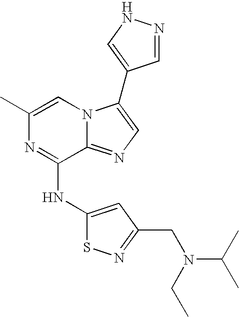

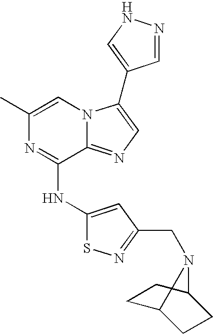

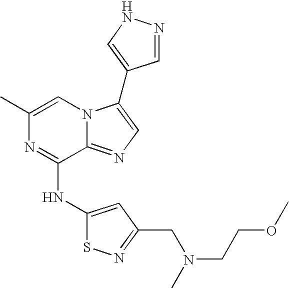

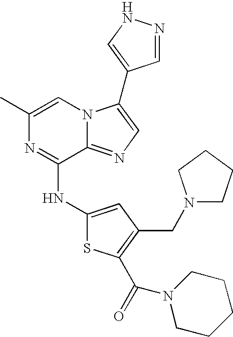

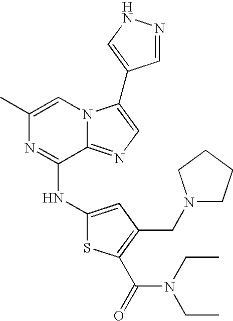

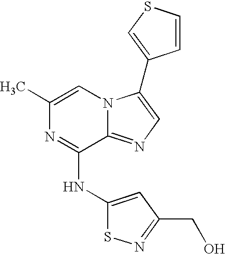

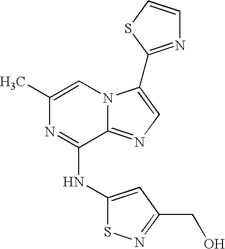

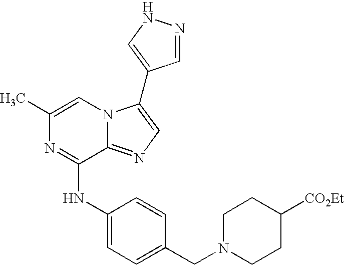

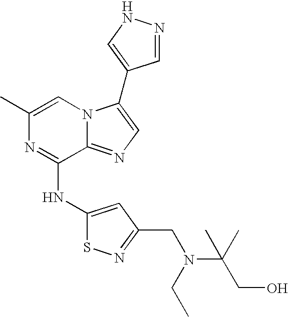

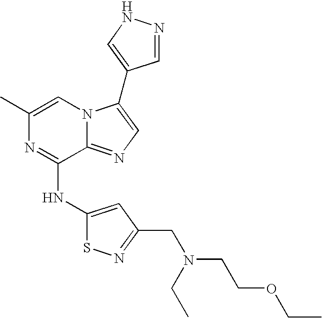

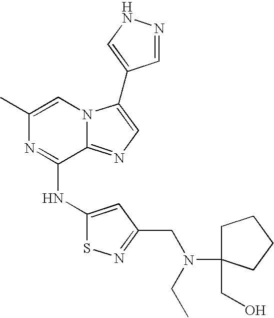

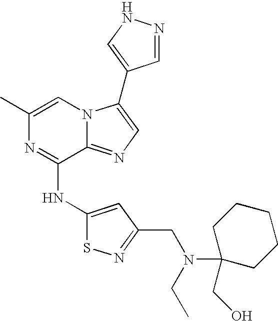

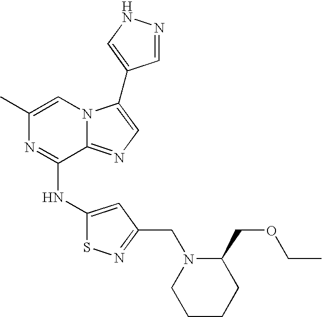

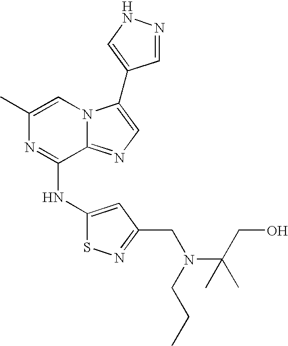

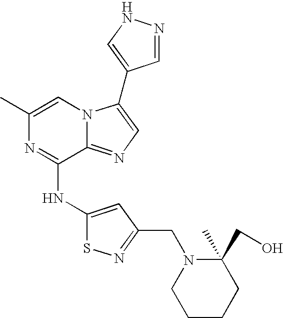

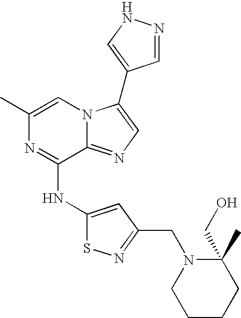

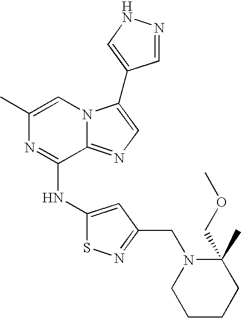

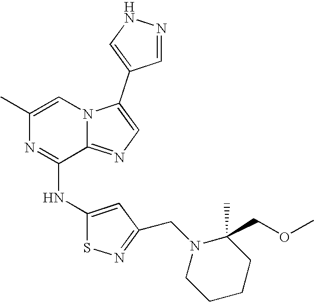

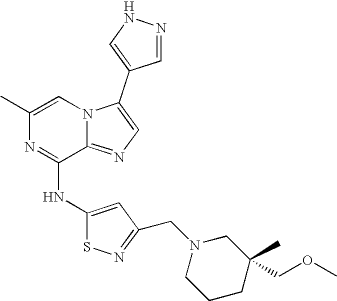

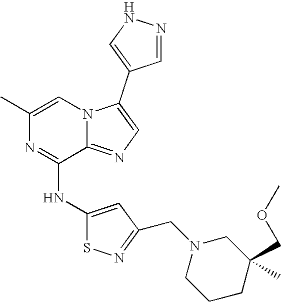

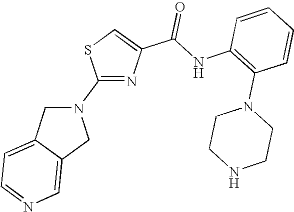

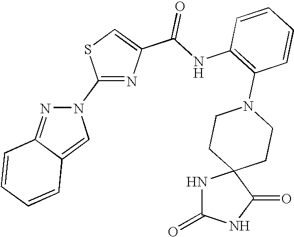

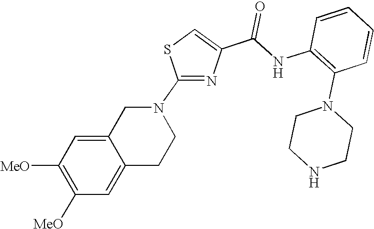

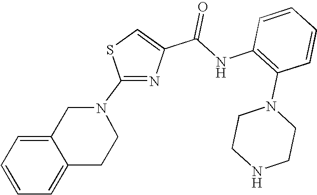

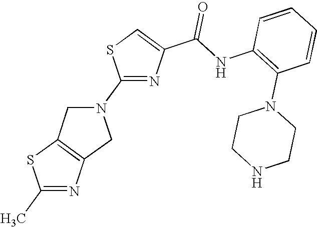

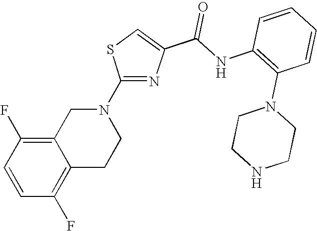

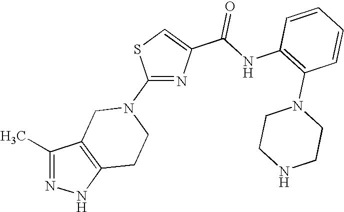

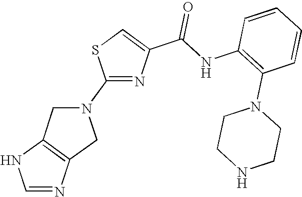

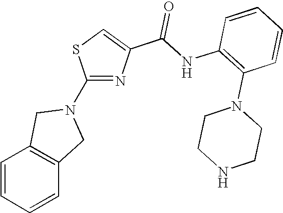

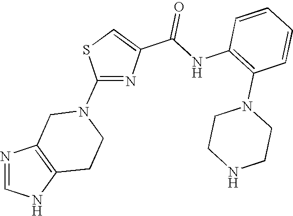

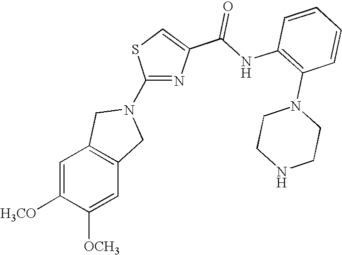

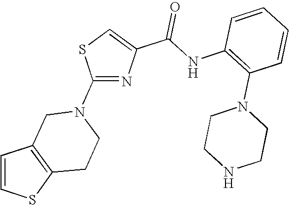

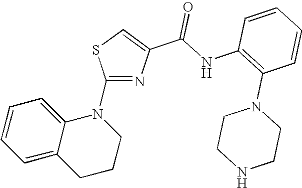

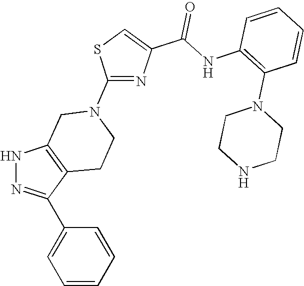

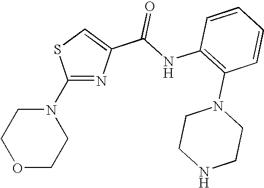

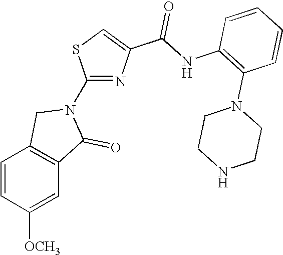

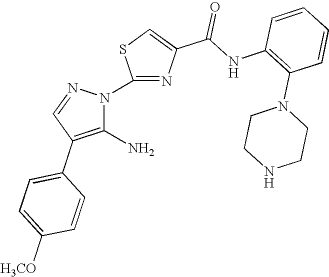

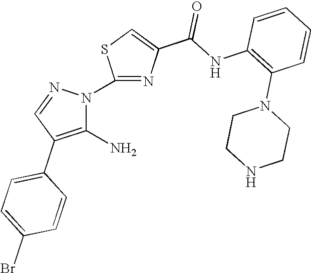

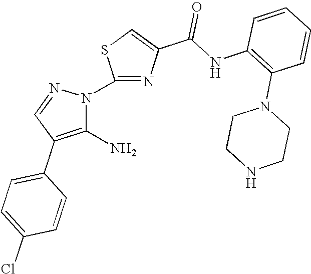

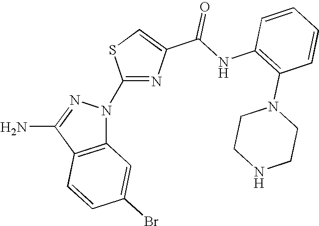

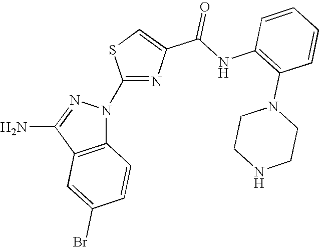

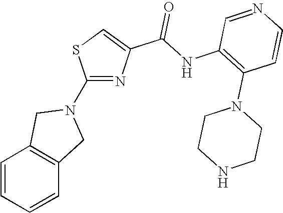

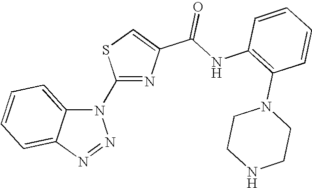

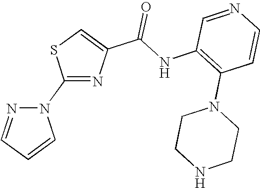

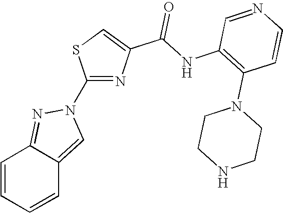

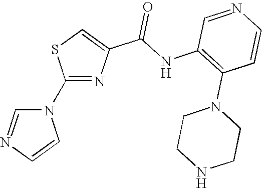

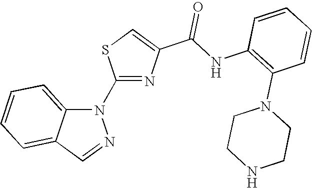

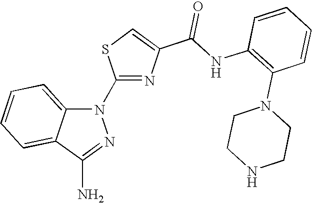

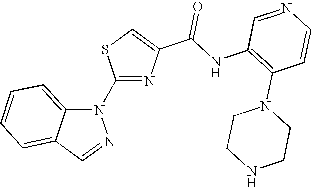

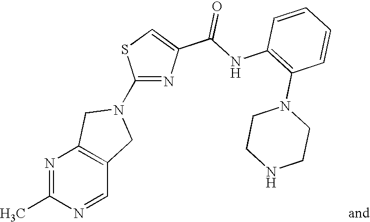

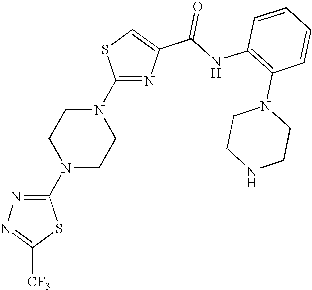

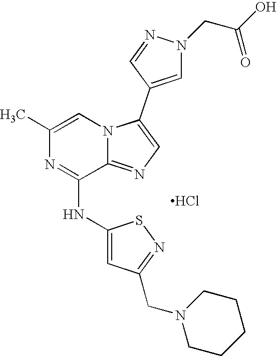

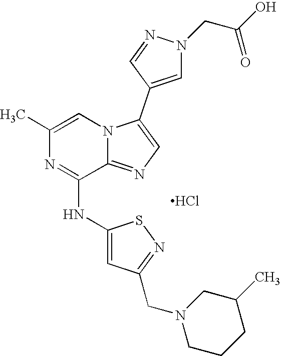

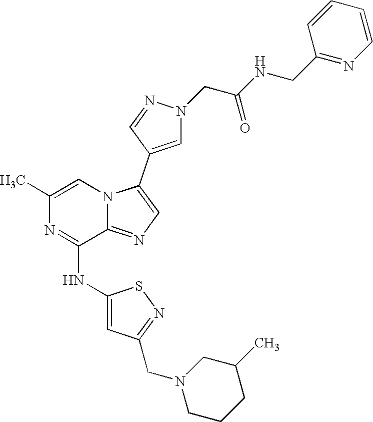

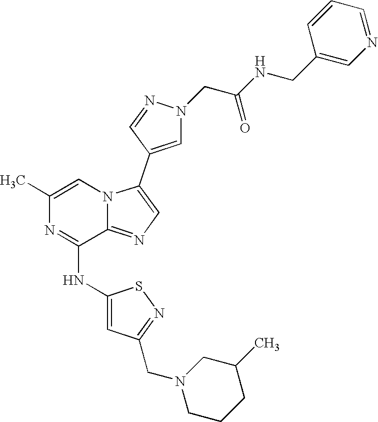

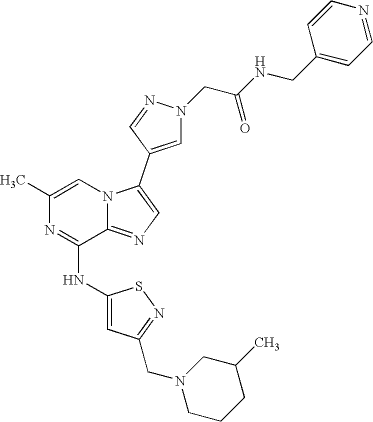

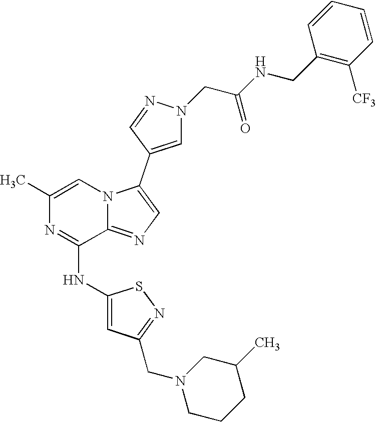

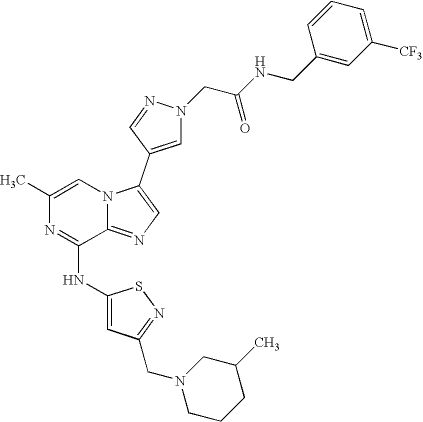

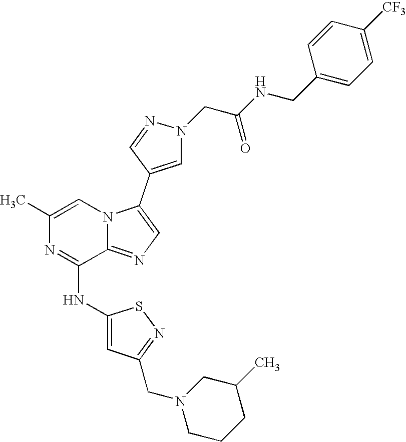

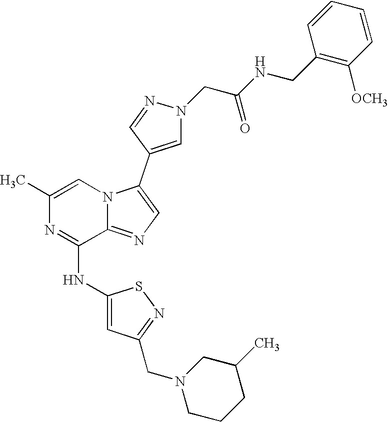

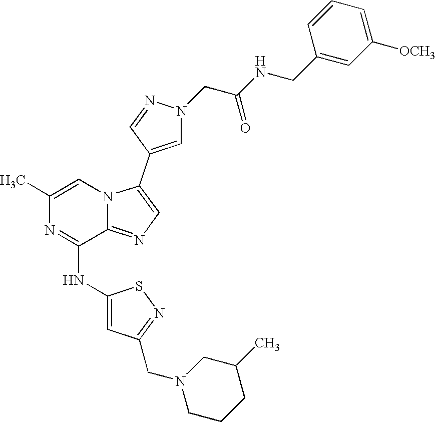

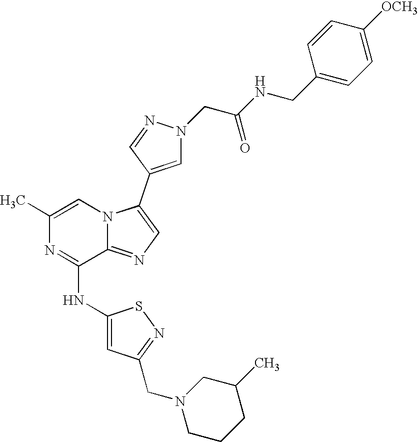

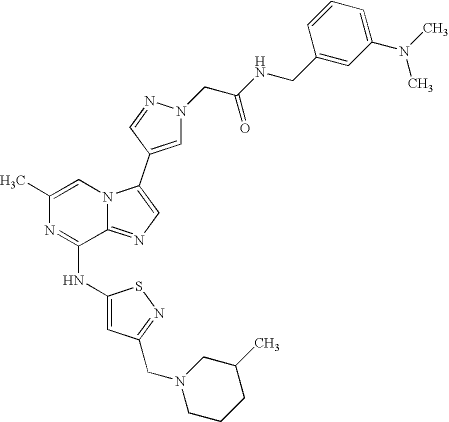

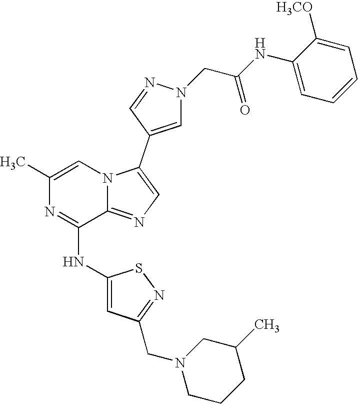

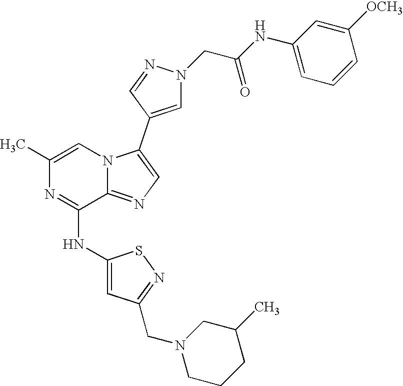

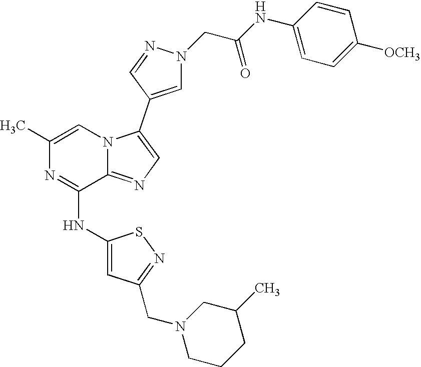

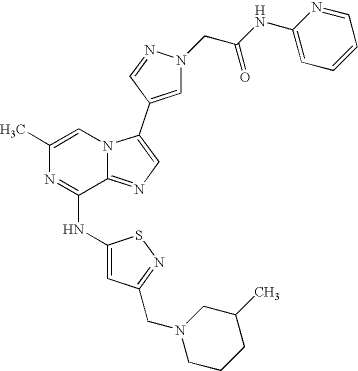

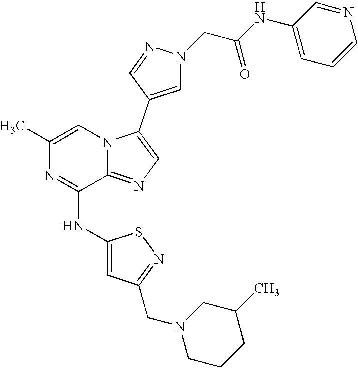

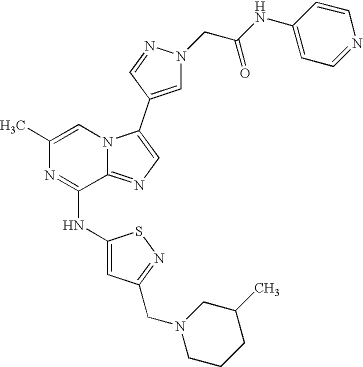

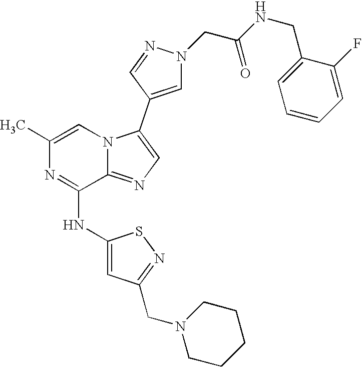

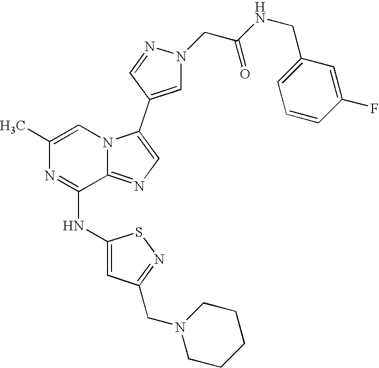

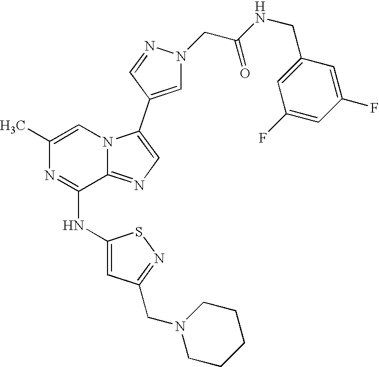

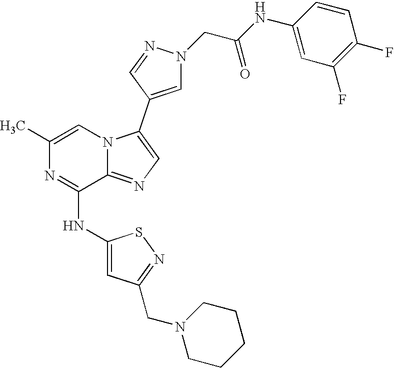

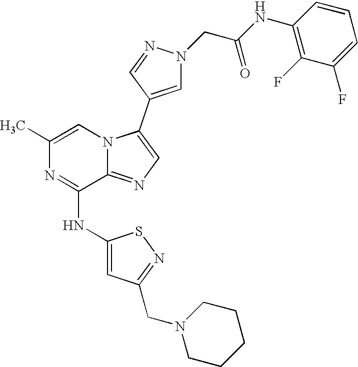

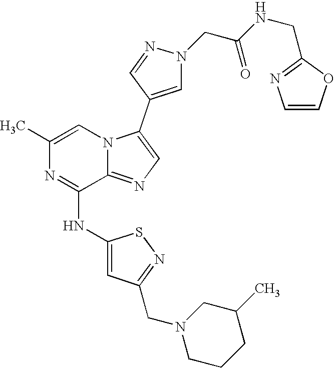

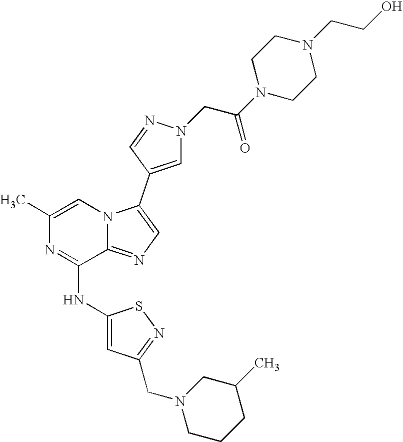

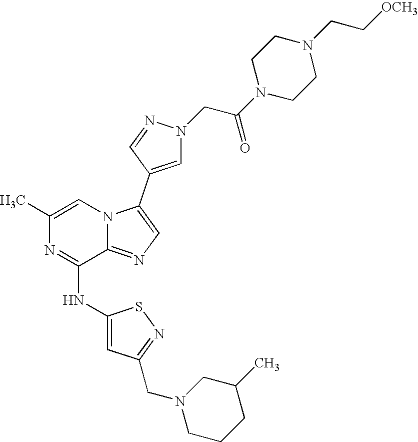

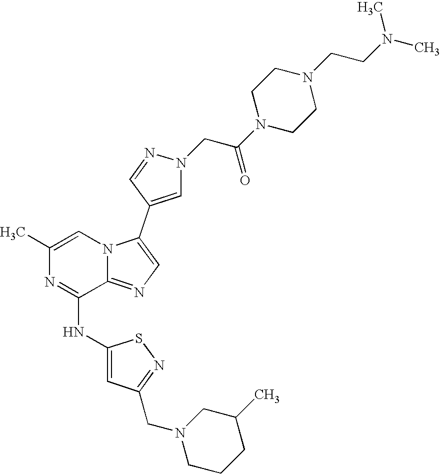

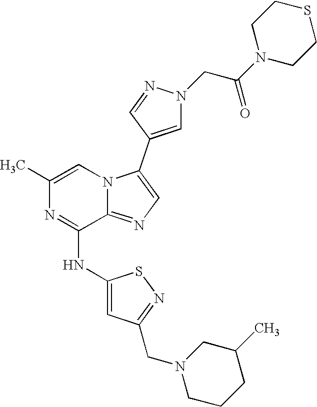

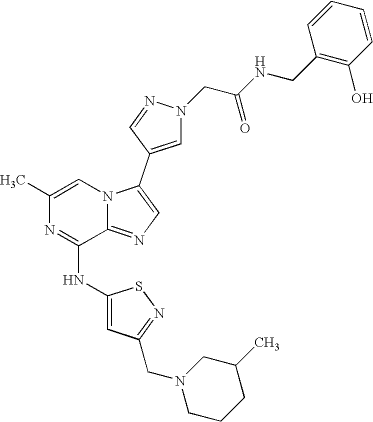

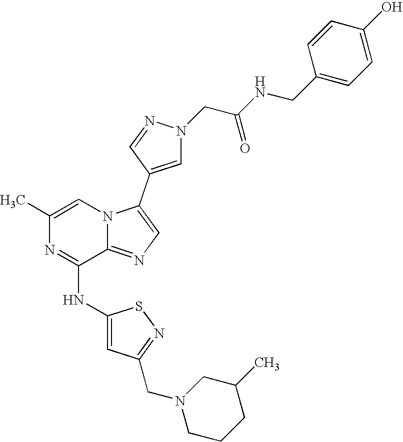

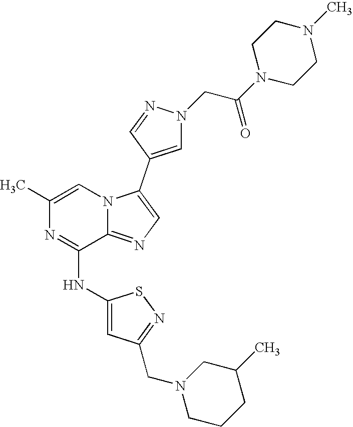

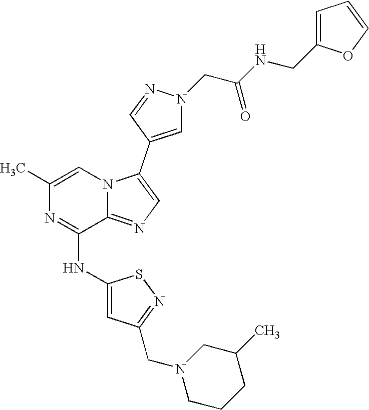

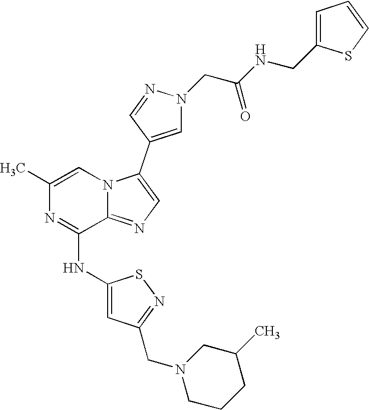

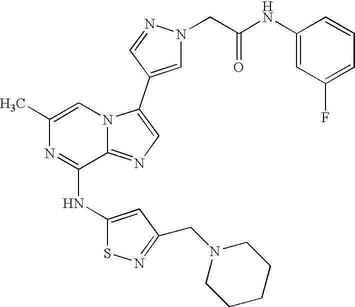

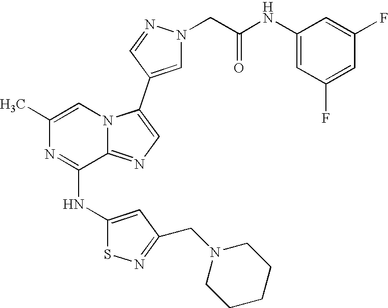

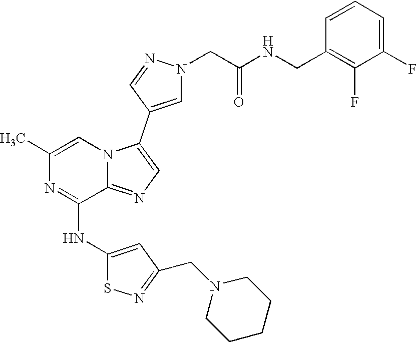

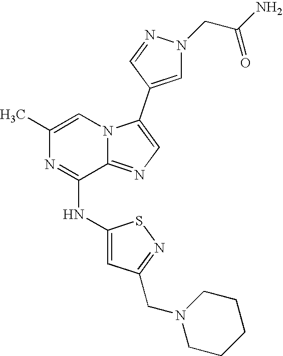

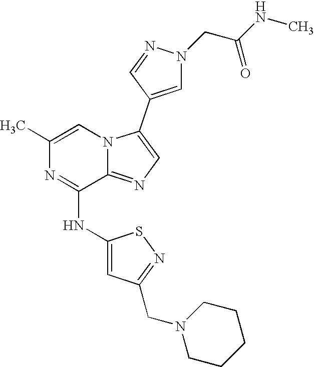

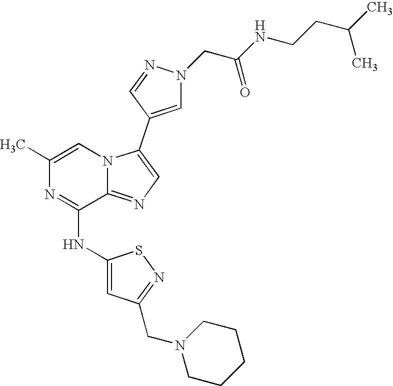

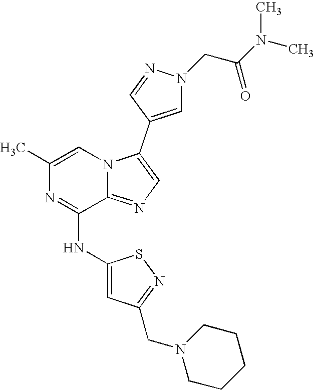

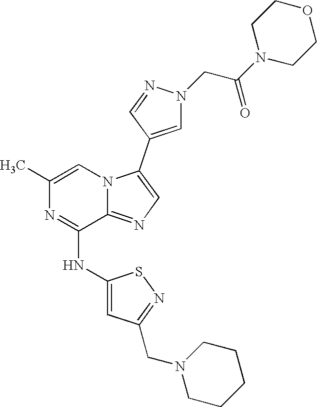

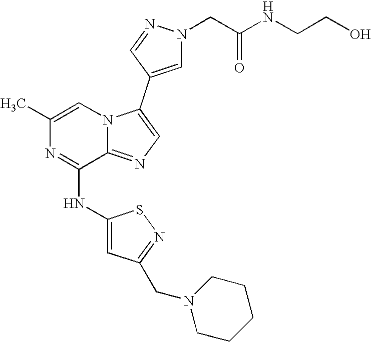

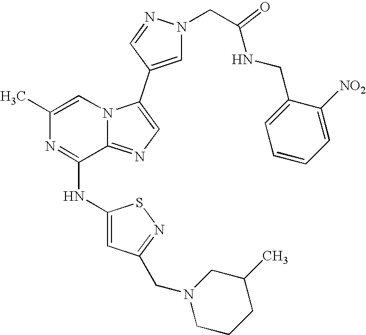

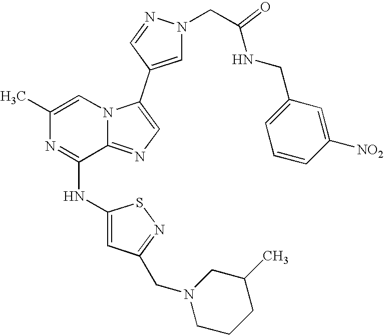

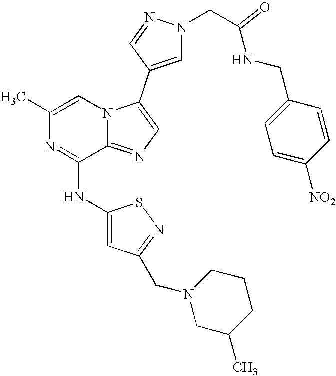

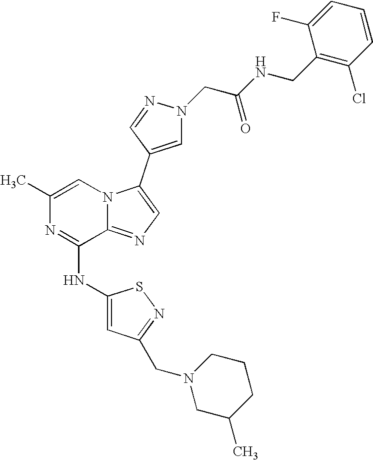

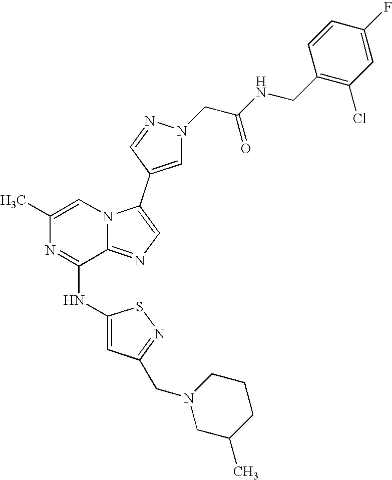

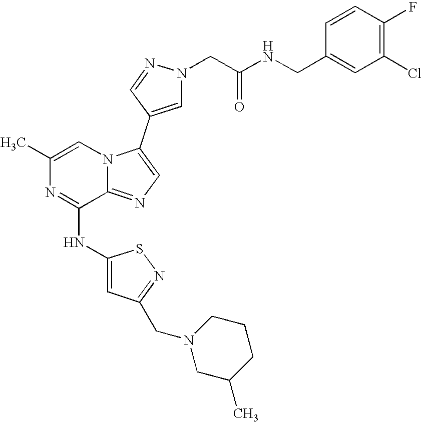

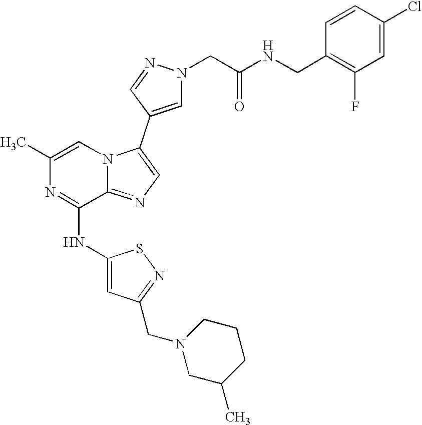

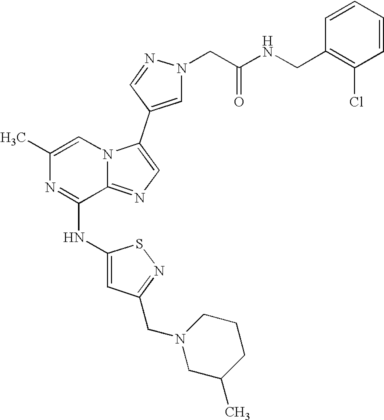

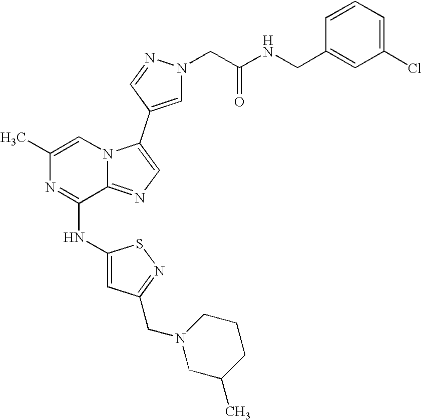

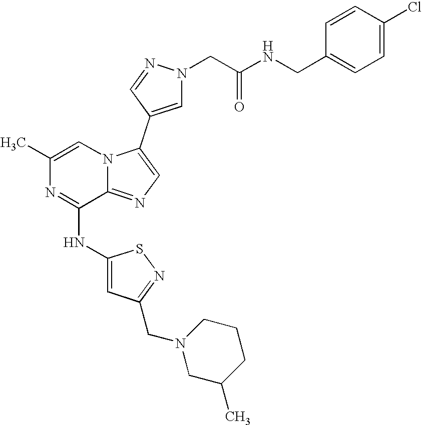

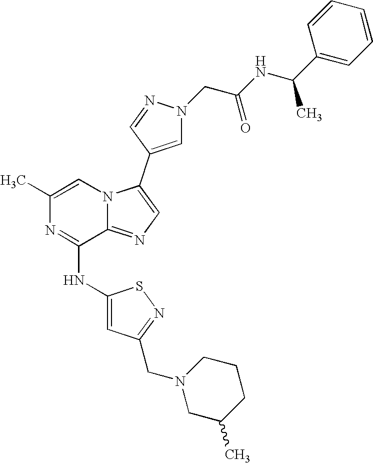

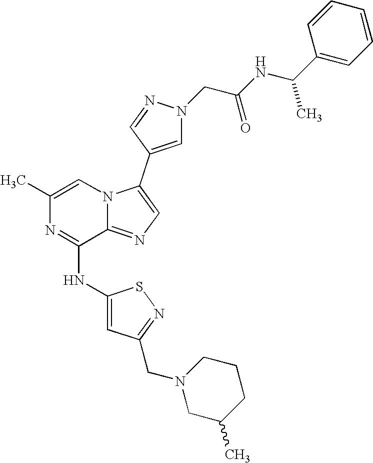

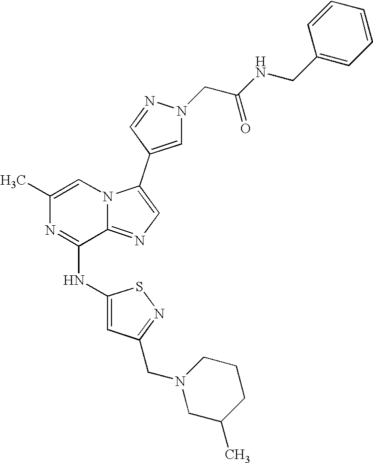

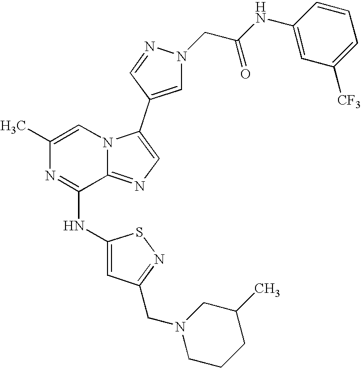

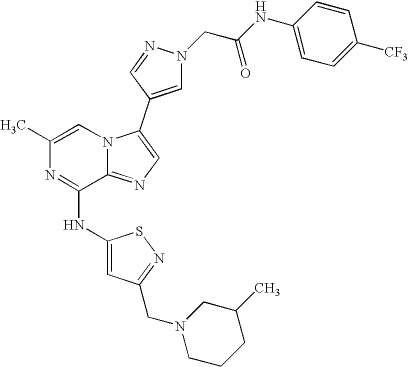

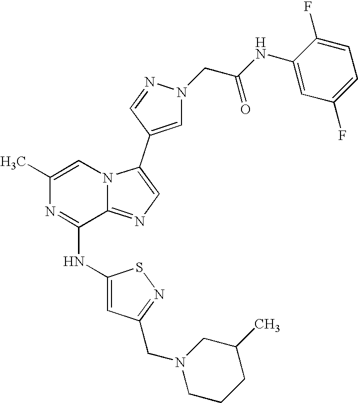

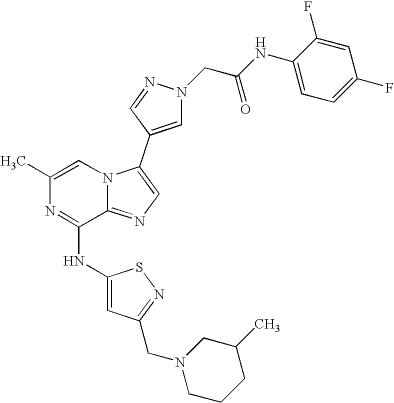

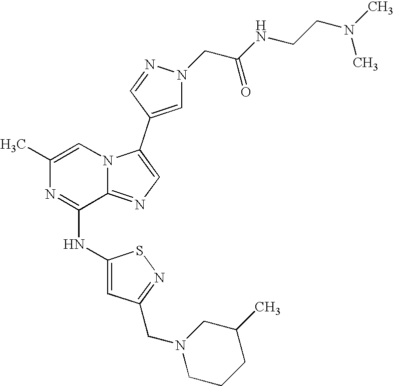

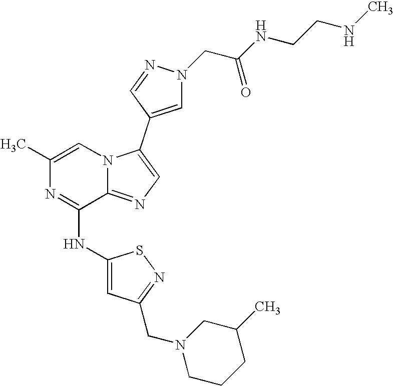

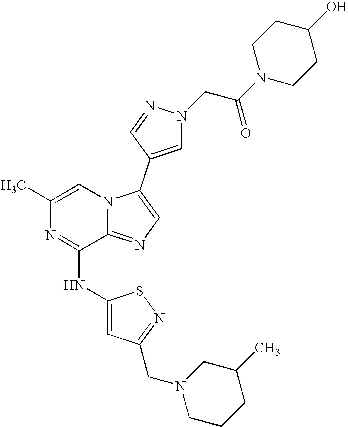

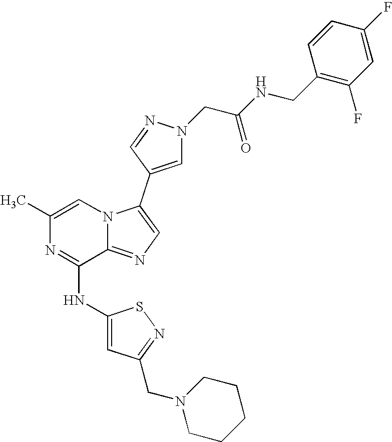

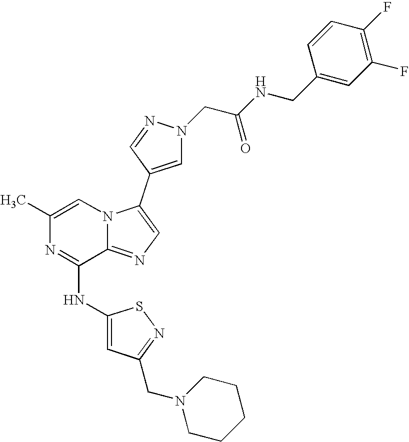

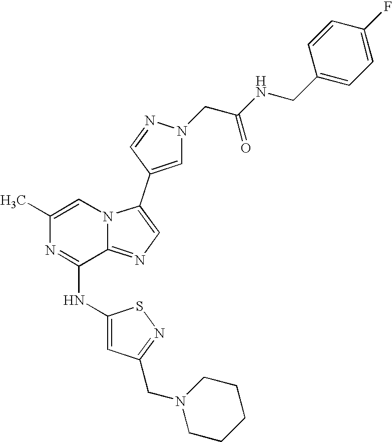

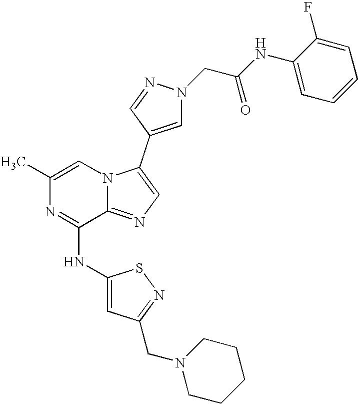

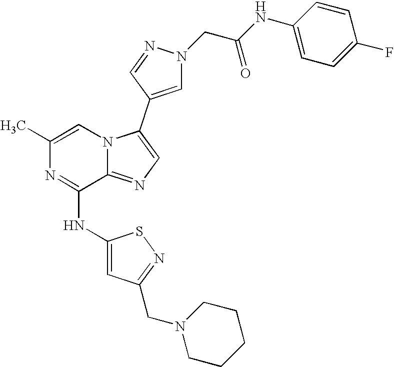

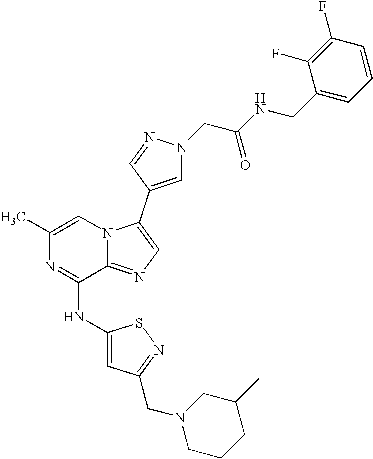

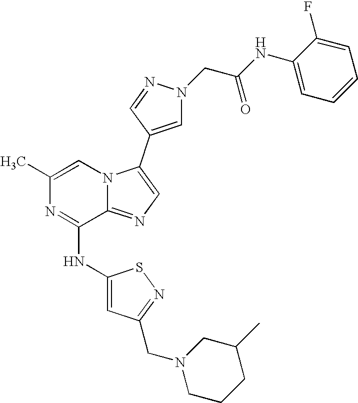

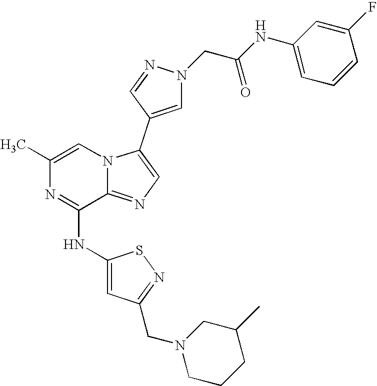

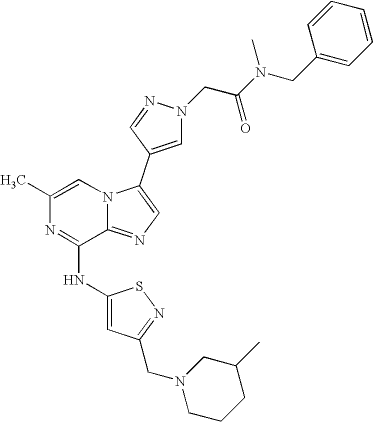

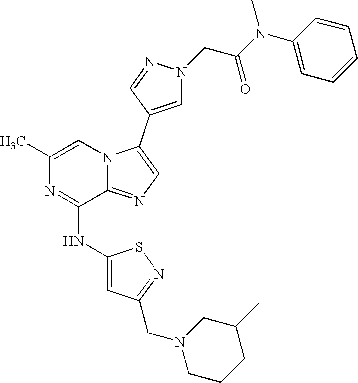

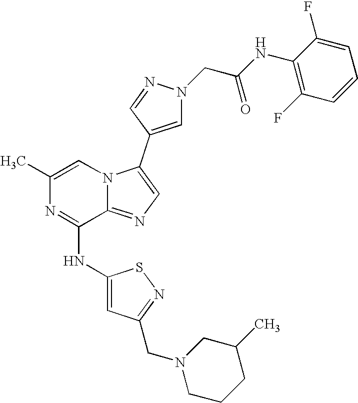

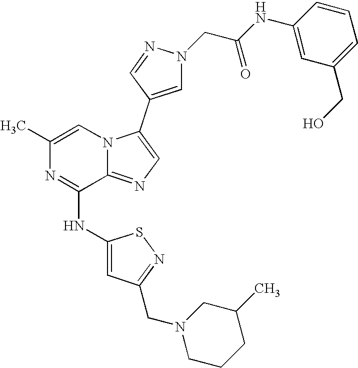

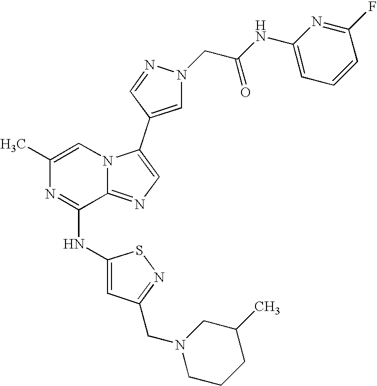

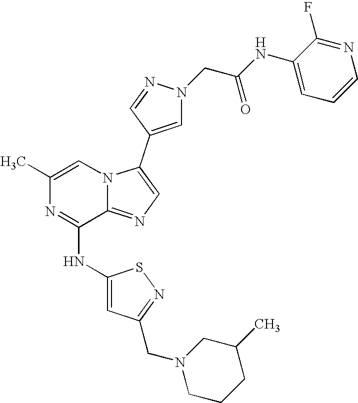

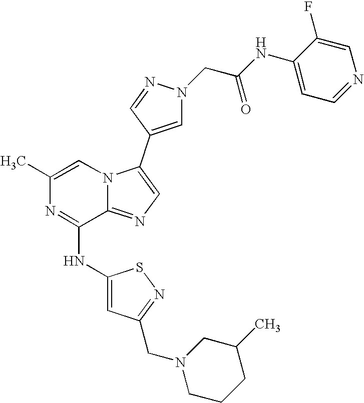

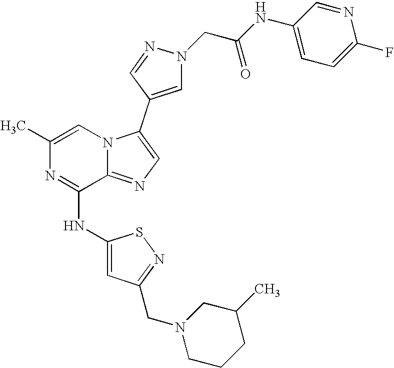

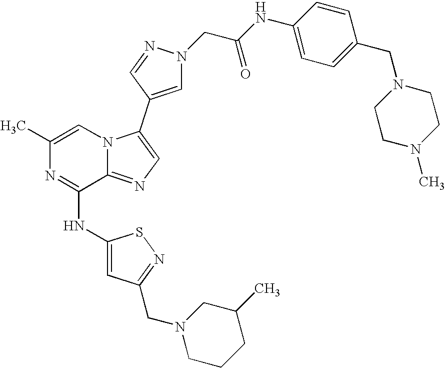

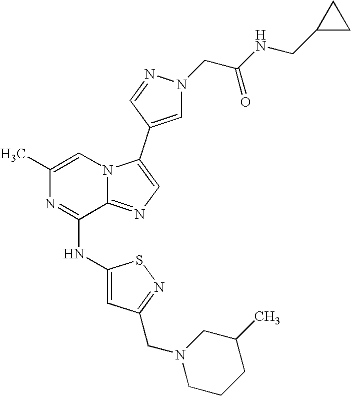

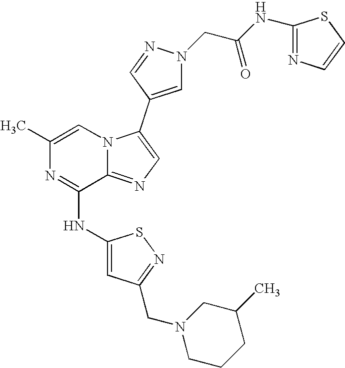

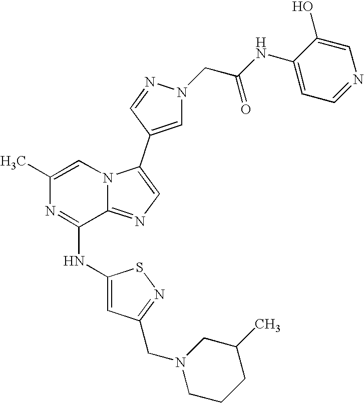

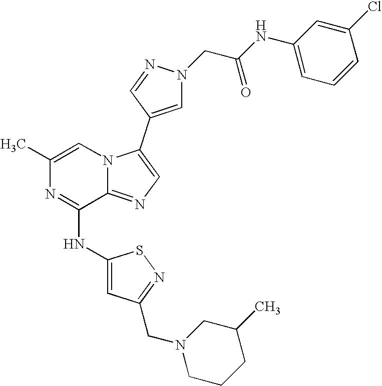

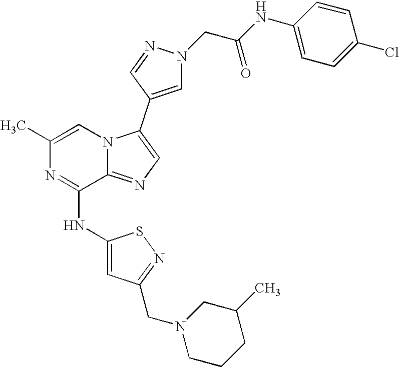

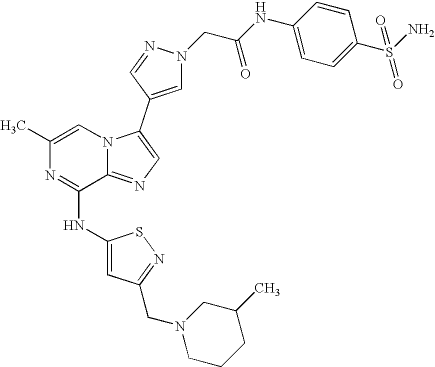

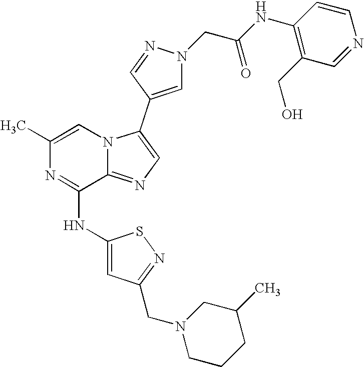

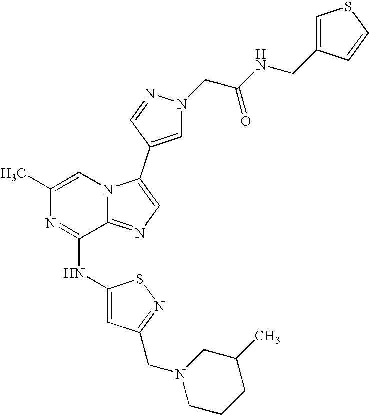

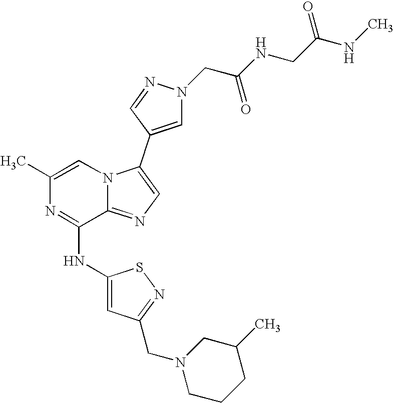

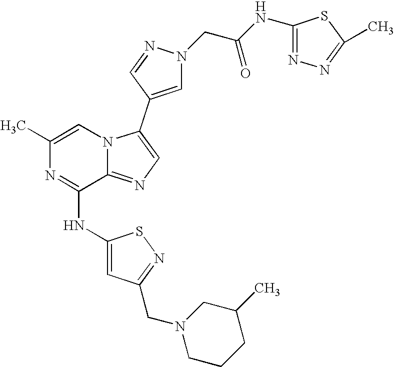

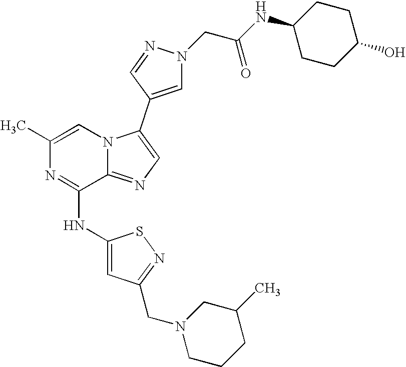

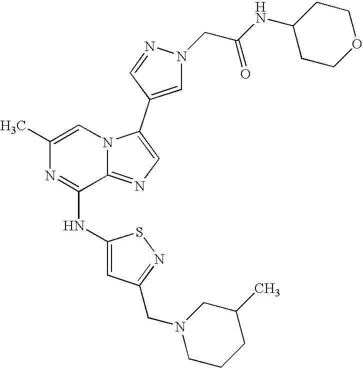

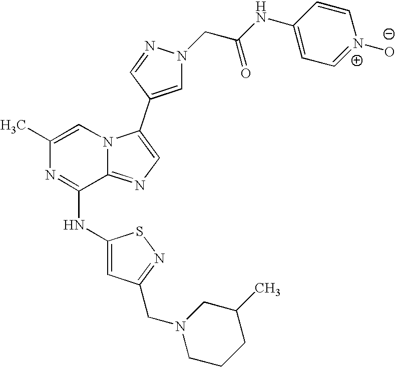

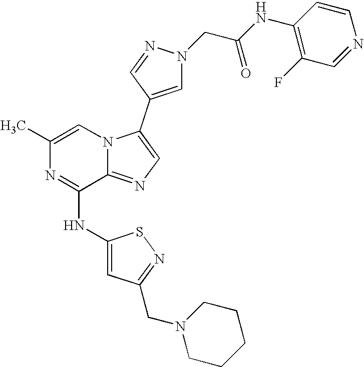

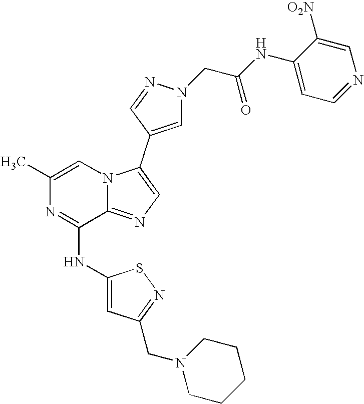

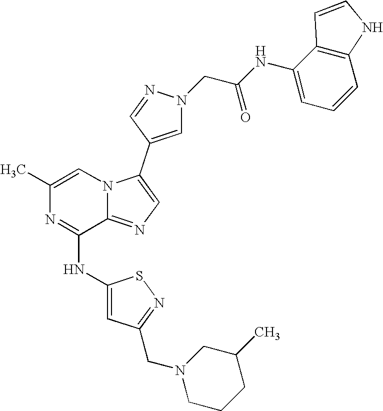

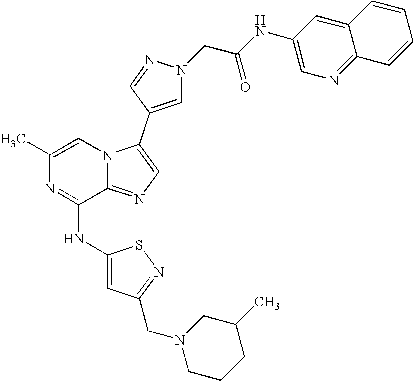

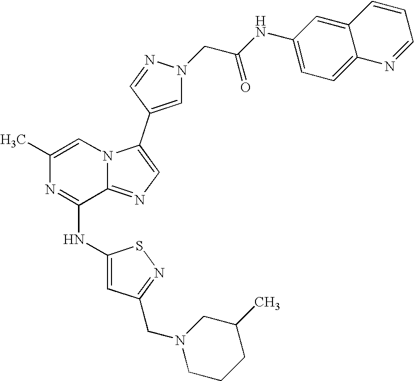

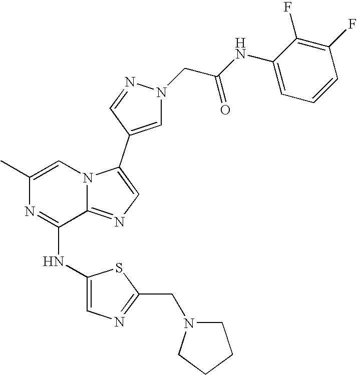

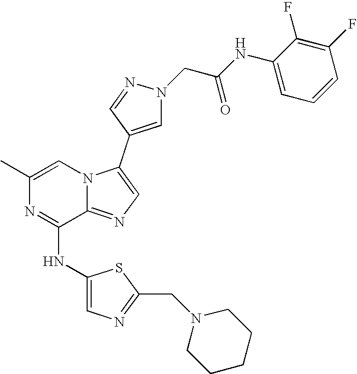

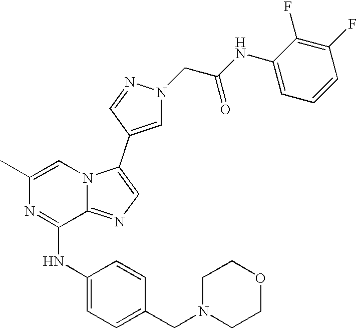

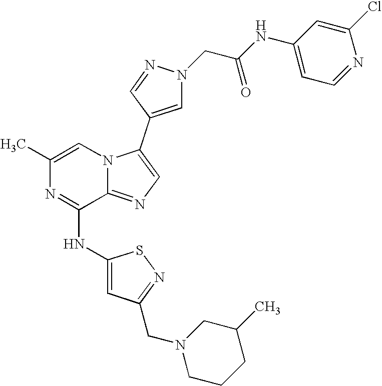

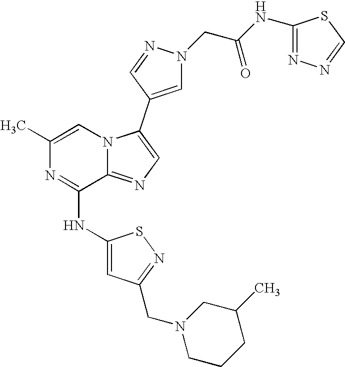

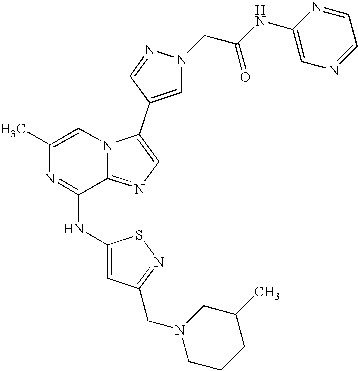

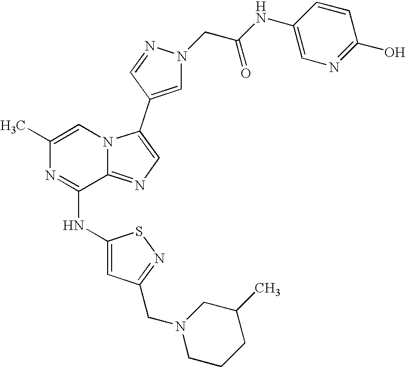

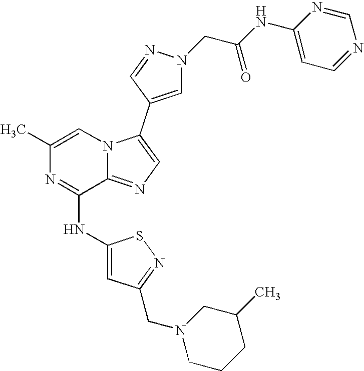

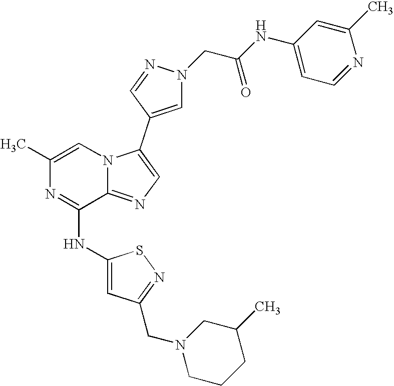

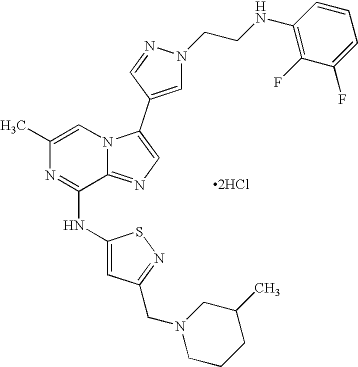

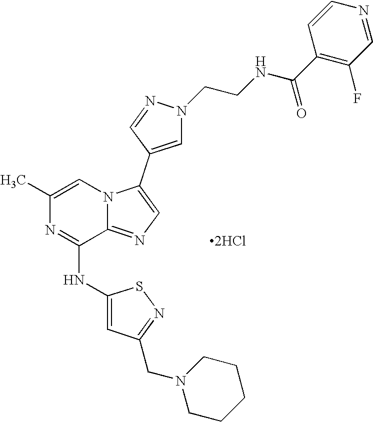

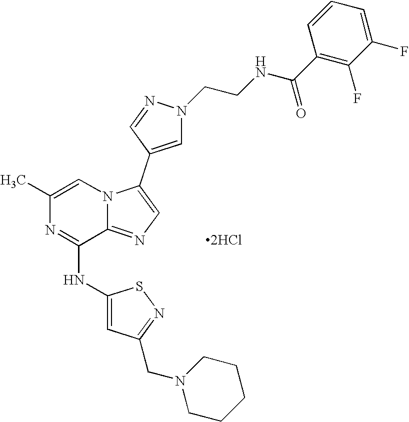

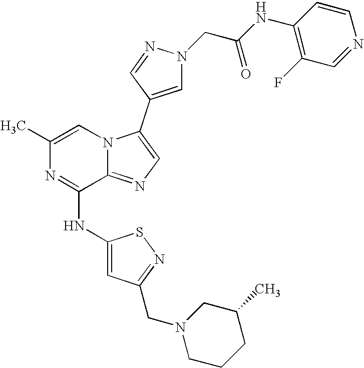

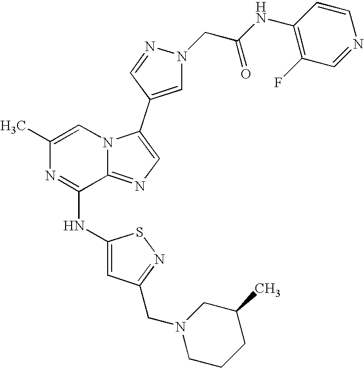

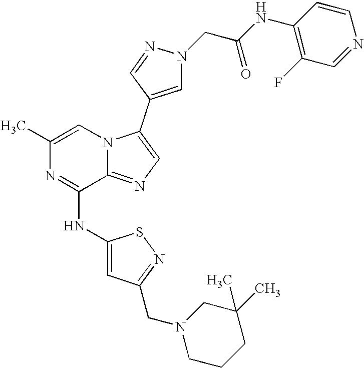

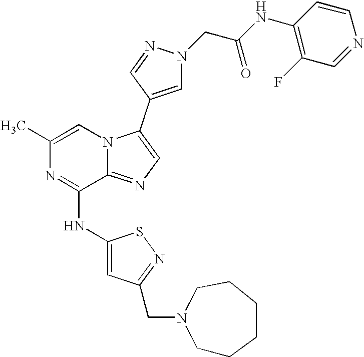

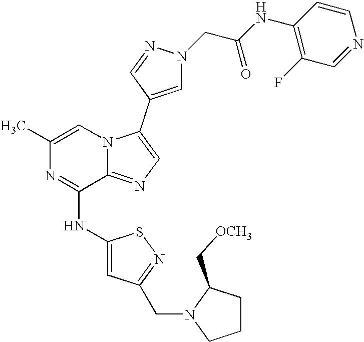

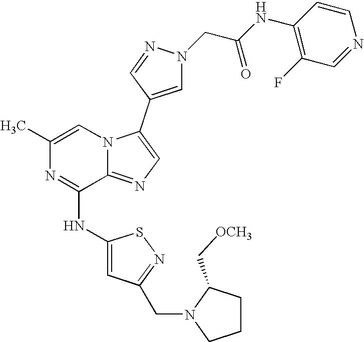

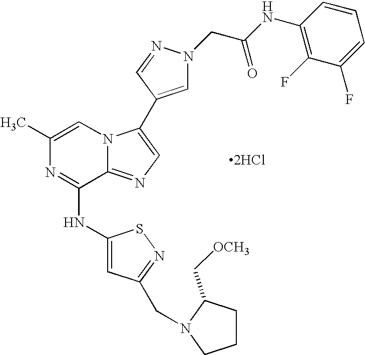

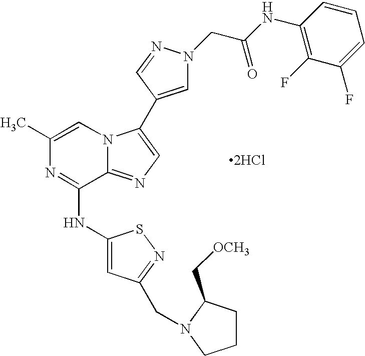

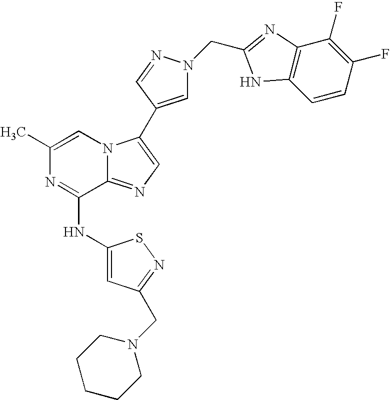

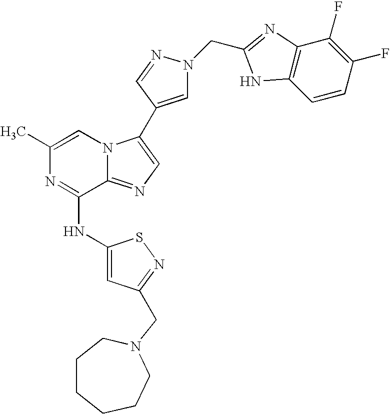

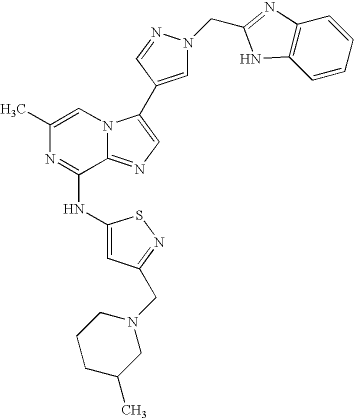

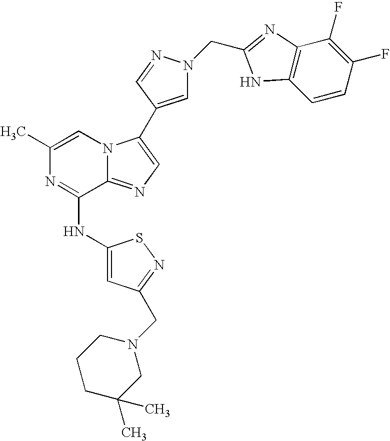

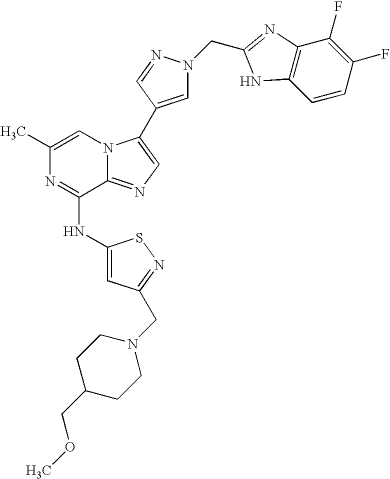

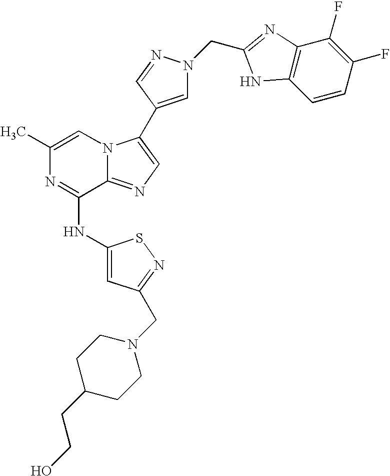

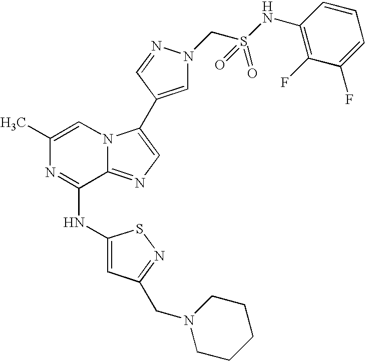

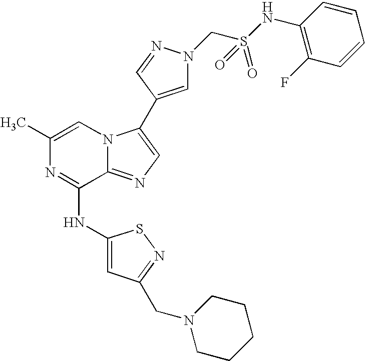

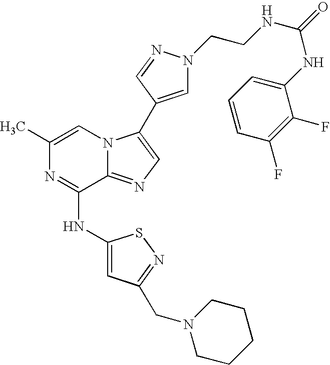

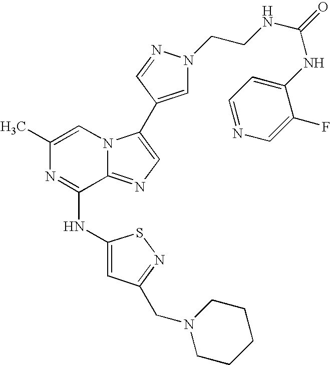

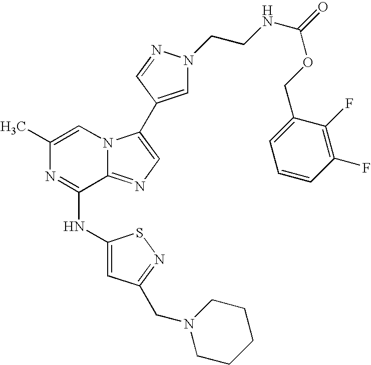

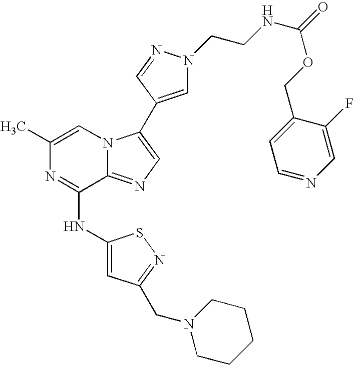

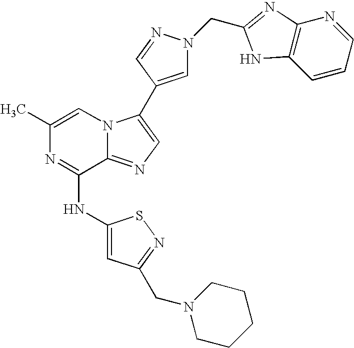

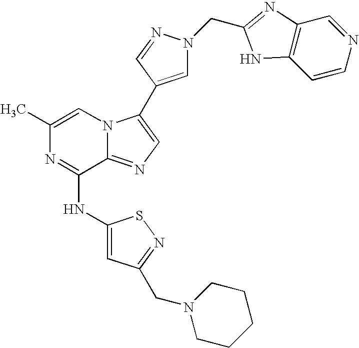

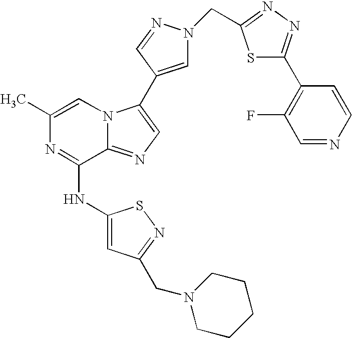

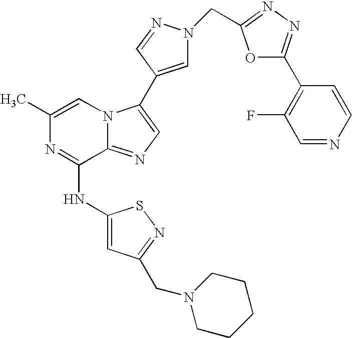

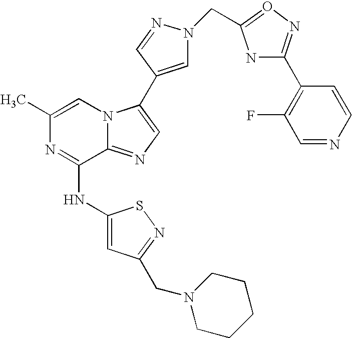

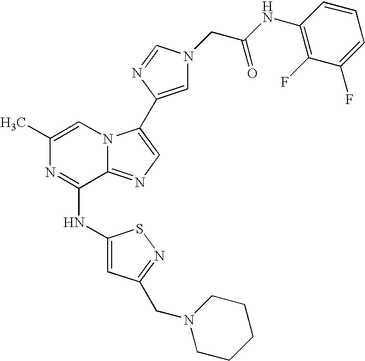

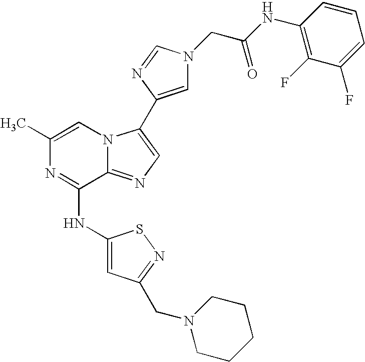

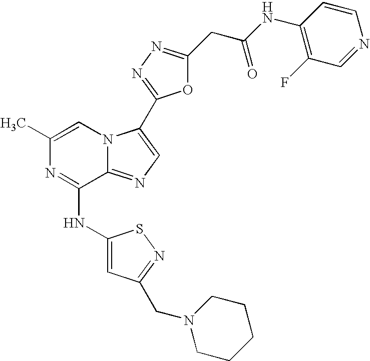

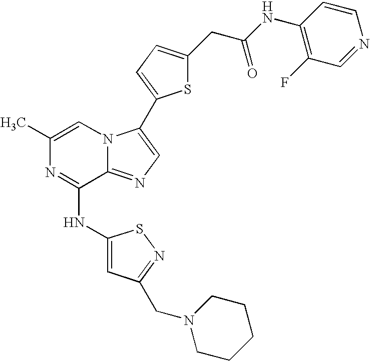

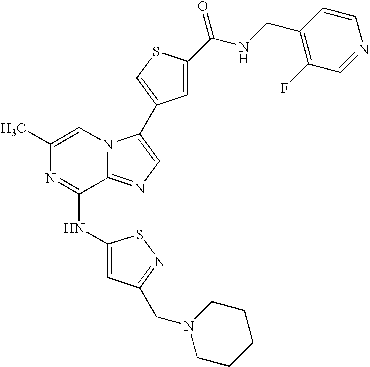

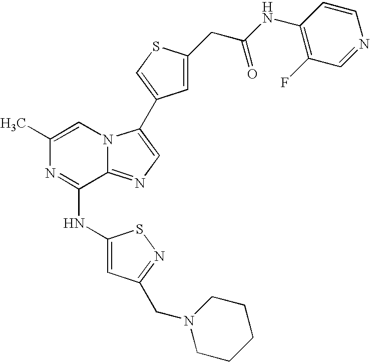

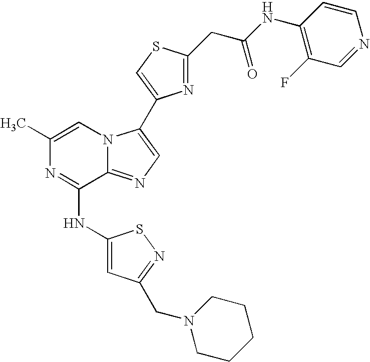

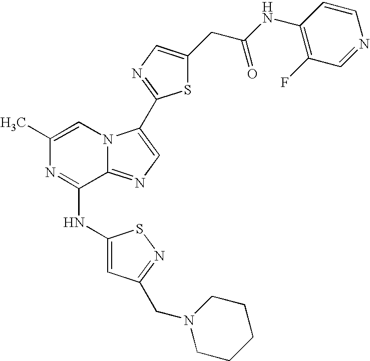

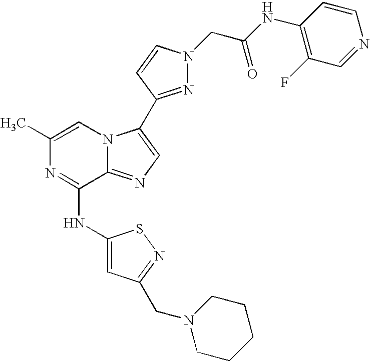

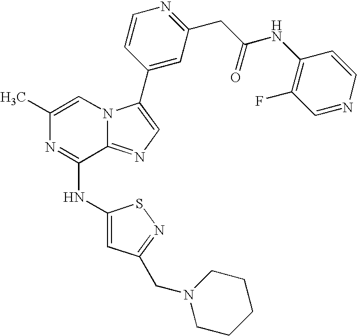

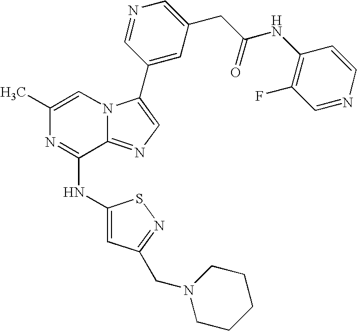

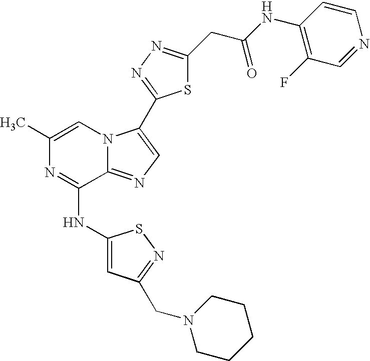

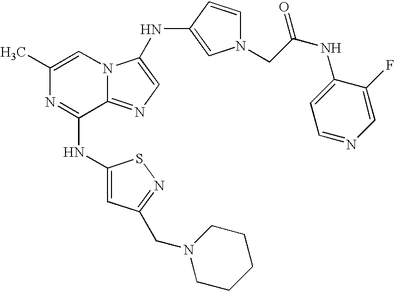

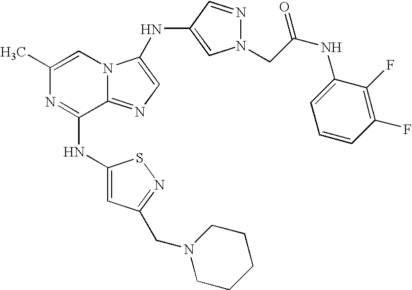

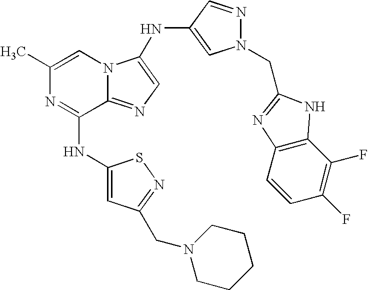

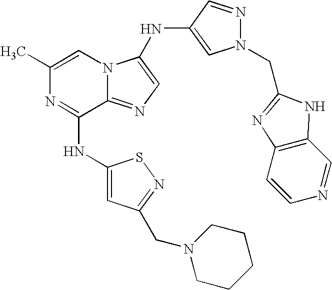

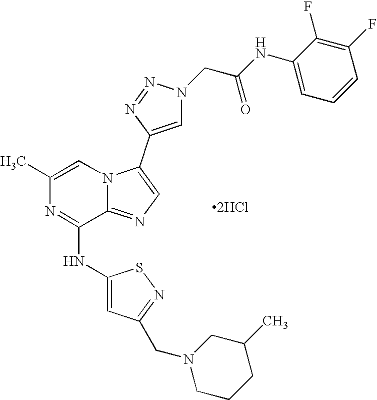

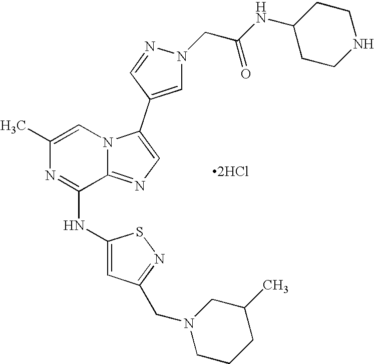

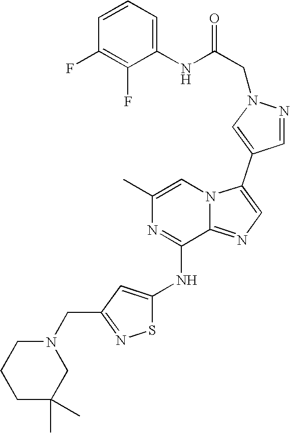

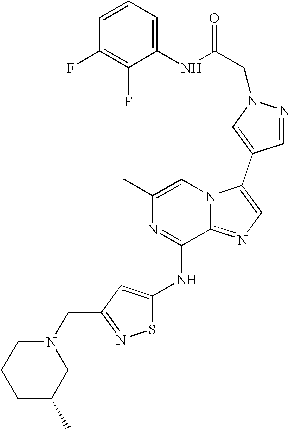

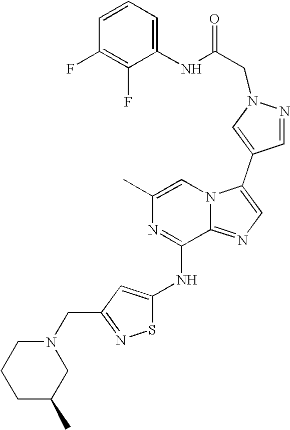

or pharmaceutically acceptable salts, solvates, esters or prodrugs thereof. The compounds listed in Table 2 above are disclosed in the following references WO2007/058942 filed Nov. 8, 2006, WO2004/026877, filed Sep. 19, 2003, WO2007/058873, filed Nov. 8, 2006, U.S. Appln. No. 60/943,999, filed Jun. 14, 2007, U.S. Application Serial No. 60/987,932, filed Nov. 14, 2007 and U.S. Appln. No. 60/855,421, filed Oct. 31, 2006, as well as from U.S. provisional patent application Ser. No. ______(with Attorney Docket No. OC06760L01), filed of even date herewith.

As used above, and throughout this disclosure, the following terms, unless otherwise indicated, shall be understood to have the following meanings, including any possible substitutions of the stated groups or moieties:

“Antimitotic agent” is a compound that inhibits cell growth by stopping cell division. These compounds specifically inhibit a cell in mitosis. These include compounds that target the cell's microtubules, centrosome, or spindles.

“Endoreduplication” is the process in a cell where there is an accumulation of polyploidy or aneuploidy DNA as a result of cell cycle rounds in the absence of cytokinesis (physical cell division).

“Polyploidy” is a cell possessing more DNA than two complete sets of chromosomes.

“Aneuploidy” is a cell containing abnormal chromosome content.

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more, lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl),

—NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more, lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more, lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Bridged cyclic ring” is a hydrocarbon ring such as cycloalkyl, cycloalkenyl, or aryl or heteroatom containing ring such as, heterocyclyl, heterocyclenyl, or heteroaryl as described herein, that contains a bridge, which is a valence bond or an atom or an unbranched chain of atoms connecting two different parts of the ring. The two tertiary carbon atoms connected through the bridge are termed “bridgeheads”.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contain at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, amide, —CHO, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogen on two adjacent carbon atoms (one H on each carbon) on a ring system. Examples of such moiety are methylene dioxy, ethylenedioxy, —C(CH₃)₂— and the like which form moieties such as, for example:

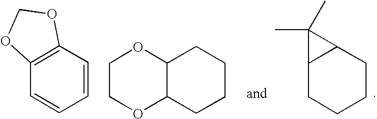

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogen on the same carbon atom on a ring system. Example of such moiety is pyrrolidone:

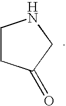

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogen on the same carbon atom on a ring system. Example of such moiety is pyrrolidinone:

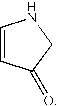

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

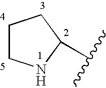

there is no —OH attached directly to carbons marked 2 and 5.

It should also be noted that tautomeric forms such as, for example, the moieties:

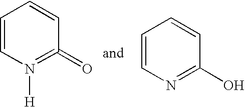

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Spiro ring systems” have two or more rings linked by one common atom. Preferred spiro ring systems include spiroheteroaryl, spiroheterocyclenyl, spiroheterocyclyl, spirocycloalkyl, spirocyclenyl, and spiroaryl. The spiro ring systems can be optionally substituted by one or more ring system substituents, wherein “ring system substituent” is as defined above. Non-limiting examples of suitable spiro ring systems include

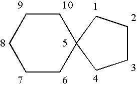

spiro[4.5]decane,

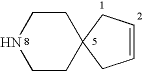

8-azaspiro[4.5]dec-2-ene, and

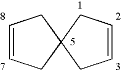

spiro[4.4]nona-2,7-diene.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogen on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(S) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in any of Formulas A-J, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of any of Formulas A-J or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of any of Formulas A-J or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of any of Formulas A-J contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of any of Formulas A-J incorporates an amine functional group, a prodrug can be formed by the replacement of a hydrogen atom in the amine group with a group such as, for example, R-carbonyl, RO-carbonyl, NRR′-carbonyl where R and R¹ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of Solvates is Generally Known. Thus, for Example, M. Caira et al, J. Pharmaceutical Sci., 93(3), 601-611 (2004) describes the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of any of Formulas A-J can form salts which are also within the scope of this invention. Reference to a compound of any of Formulas A-J herein is understood to include reference to salts thereof, unless otherwise indicated. The term “salt(S)”, as employed herein, denotes acidic salts formed with inorganic and/or organic acids, as well as basic salts formed with inorganic and/or organic bases. In addition, when a compound of any of Formulas A-J contains both a basic moiety, such as, but not limited to a pyridine or imidazole, and an acidic moiety, such as, but not limited to a carboxylic acid, zwitterions (“inner salts”) may be formed and are included within the term “salt(S)” as used herein. Pharmaceutically acceptable (i.e., non-toxic, physiologically acceptable) salts are preferred, although other salts are also useful. Salts of the compounds of any of Formulas A-J may be formed, for example, by reacting a compound of any of Formulas A-J with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄allyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of any of Formulas A-J, and salts, solvates, esters and prodrugs thereof, may exist in their tautomeric form (for example, as an amide or imino ether). All such tautomeric forms are contemplated herein as part of the present invention.

The compounds of any of Formulas A-J may contain asymmetric or chiral centers, and, therefore, exist in different stereoisomeric forms. It is intended that all stereoisomeric forms of the compounds of any of Formulas A-J as well as mixtures thereof, including racemic mixtures, form part of the present invention. In addition, the present invention embraces all geometric and positional isomers. For example, if a compound of any of Formulas A-J incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of any of Formulas A-J may be atropisomers (e.g., substituted biaryls) and are considered as part of this invention. Enantiomers can also be separated by use of chiral HPLC column.

It is also possible that the compounds of any of Formulas A-J may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of any of Formulas A-J incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of any of Formulas A-J (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of any of Formulas A-J can generally be prepared by following procedures analogous to those disclosed in the Schemes and/or in the Examples herein below, by substituting an appropriate isotopically labeled reagent for a non-isotopically labeled reagent.

Polymorphic forms of the compounds of any of Formulas A-J, and of the salts, solvates, esters and prodrugs of the compounds of any of Formulas A-J, are intended to be included in the present invention.

More specifically, the combination treatment of the present invention comprising first administration of the anti-mitotic agent described above, followed by administration of the compounds of any of Formulas E-K can be useful in the treatment of a variety of cancers, including (but not limited to) the following:

tumor of the bladder, breast (including BRCA-mutated breast cancer), colorectal, colon, kidney, liver, lung (including small cell lung cancer and non-small cell lung cancer), head and neck, esophagus, bladder, gall bladder, ovary, pancreas, stomach, cervix, thyroid, prostate, and skin, including squamous cell carcinoma;

leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma and Burkett's lymphoma;

chronic lymphocytic leukemia (“CLL”),

acute and chronic myelogenous leukemia, myelodysplastic syndrome and promyelocytic leukemia;

fibrosarcoma, rhabdomyosarcoma;

mantle cell lymphoma, myeloma;

astrocytoma, neuroblastoma, glioblastoma, malignant glial tumors, hepatocellular carcinoma, gastrointestinal stromal tumors (“GIST”) and schwannomas;

melanoma, multiple myeloma, seminoma, teratocarcinoma, osteosarcoma, xenoderoma pigrnentosum, keratoctanthoma, thyroid follicular cancer and Kaposi's sarcoma.

The combination treatment of the present invention can be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The combination treatment of the present invention may also be useful in inhibiting tumor angiogenesis and metastasis.

Non-limiting examples of suitable anti-mitotic agent is selected from the group consisting of a taxotere (docetaxel), taxol (paclitaxel), KSP inhibitors (such as, for example, those of Formulas A-D described herein or Ispinesib SB-715992 (Cytokinetics), and centrosome associated protein E (“CENP-E”) inhibitor (e.g., GSK-923295), ABRAXANE® (Abraxis BioScience Inc and AstraZeneca Pharmaceuticals), and epothilone including epothilone A, B or D.

At least one aurora kinase inhibitor of any of Formulas E-K should be administered following administration of at least one anti-mitotic agent or when a combination formulation of at least one anti-mitotic agent and at least one aurora kinase inhibitor is administered, the aurora kinase inhibitor would be time released after the release of the anti-mitotic agent. Such techniques are within the skills of persons skilled in the art as well as attending physicians.

Accordingly, in an aspect, this invention includes combinations comprising an amount of one or more anti-mitotic agents listed above and an amount of at least one compound of any of Formulas E-K, or a pharmaceutically acceptable salt, solvate, ester or prodrug thereof, wherein the combination also includes a time release agent that releases the aurora kinase inhibitor after the antimitotic agent, in amounts and at a time that results in a desired therapeutic effect.

Another aspect of the present invention is a method of inhibiting one or more Aurora kinases in a patient in need thereof, comprising administering to the patient, a therapeutically effective amount of at least one compound of any of Formulas E-K or a pharmaceutically acceptable salt, solvate, ester or prodrug thereof following administration of one or more anti-mitotic agents described above.

Another aspect of the present invention is a method of treating, or slowing the progression of, a disease associated with one or more Aurora kinases in a patient in need thereof, comprising administering a therapeutically effective amount of at least one compound of any of Formulas E-K or a pharmaceutically acceptable salt, solvate, ester or prodrug thereof following administration of an anti-mitotic agent described above.

Yet another aspect of the present invention is a method of treating one or more diseases associated with Aurora kinase, comprising administering to a mammal in need of such treatment an amount of a first compound, which is an anti-mitotic agent or a pharmaceutically acceptable salt, solvate, ester or prodrug thereof; and an amount of at least one second compound, the second compound being a compound of any of Formulas E-K, wherein the amounts of the first compound and the second compound result in a therapeutic effect.

Another aspect of the present invention is a method of treating, or slowing the progression of, a disease associated with one or more Aurora kinases in a patient in need thereof, comprising administering an anti-mitotic agent followed by a therapeutically effective amount of a pharmaceutical composition comprising in combination at least one pharmaceutically acceptable carrier and at least one compound according to any of Formulas E-K, or a pharmaceutically acceptable salt, solvate, ester or prodrug thereof.

In the above methods, the Aurora kinase to be inhibited can be Aurora B and/or Aurora A/B, A/B/C, or B/C.

The pharmacological properties of the pharmaceutical compositions which comprise separately at least one anti-mitotic agent and at least one compound of any of Formulas E-K, or a pharmaceutically acceptable salt, solvate, ester or prodrug of said compound and at least one pharmaceutically acceptable carrier may be confirmed by a number of pharmacological assays. The exemplified pharmacological assays which are described herein below have been carried out with pharmaceutical compositions according to the invention.

This invention is also directed to pharmaceutical compositions which comprise separately at least one anti-mitotic agent and at least one compound of any of Formulas E-K, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally or intravenously.

Also contemplated are delivery methods that are combinations of the above-noted delivery methods. Such methods are typically decided by those skilled in the art.

Preferably, the pharmaceutical preparation is in a unit dosage form. In such form, the preparation is subdivided into suitably sized unit doses containing appropriate quantities of the active components, e.g., an effective amount of antimotic agent and an effective amount of aurora kinase inhibitor of Formula E-J, to achieve the desired purpose.

Another aspect of this invention is a kit comprising a separately a therapeutically effective amount of at least one anti-mitotic agent and at least one compound of any of Formulas E-K, or a pharmaceutically acceptable salt, solvate, ester or prodrug of said compound and a pharmaceutically acceptable carrier, vehicle or diluent.

### SYNTHETIC EXAMPLES

The synthesis of compounds of paragraphs (d), (h), (i), (j) and (k), which are unpublished as of the filing date of the present patent application, is shown below, as reported respectively in U.S. provisional patent application Ser. Nos. 60/939,963, 60/858,244, 60/987,932 (and 60/943,999), 60/855,421 and Attorney Docket No. OC06760L01 (filed of even date herewith).

The synthesis of the compounds of paragraph (d) as described in U.S. application Ser. No. 60/939,963 is shown below.

### Method A

Standard operating procedure (SOP) For Solid Phase Synthesis of Benzimidazoles

### 4-Fluoro-3-nitrobenzoyl chloride

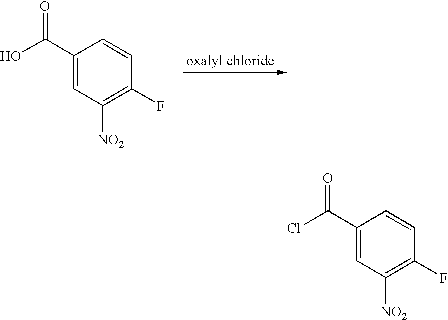

4-Fluoro-4-nitrobenzoic acid (277 mg, 1.5 mmol) was dissolved in DCM (5 mL) and oxalyl chloride was added (630 μL, 5 eq.) followed by 2 μL DMF. The solution was stirred for 1 h at rt. then concentrated to give 1.5 mmol of acid chloride which is stored under argon and must be used in the next step fresh.

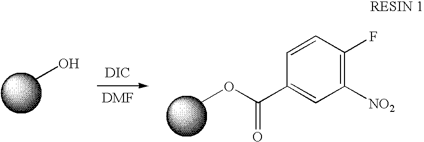

To Wang resin (3.0 mmol) was added DMF (30 mL) and 4-fluoro-3-nitrobenzoic acid (1.66 g, 9.0 mmol), DIC (1.4 mL, 9.0 mmol) and DMAP (80 mg). The mixture was stirred overnight at rt. and filtered. The resin was washed with DMF (4×), i-PrOH, DCM (3× each, Et₂O and dried in vacuo.

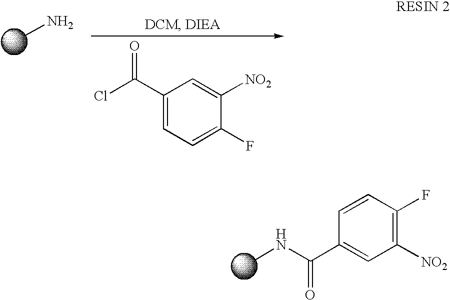

To Rink AM resin (Novabiochem, 0.4 mmol) was added a mixture of piperidine and DMF (6 mL, 50%) and the mixture was shaken for 45 min and filtered. The resin was thoroughly washed with DMF, i-PrOH, DCM (3× each), Et₂O and dried.

To the resin was added DCM (5 mL), DIEA (315 μL, 1.8 mmol) and freshly prepared 4-fluoro-3-nitrobenzoyl chloride (1.5 mmol) in 3 mL DCM. The mixture was stirred overnight at rt. then filtered. The resin was washed with DCM, i-PrOH, DCM (3× each), Et₂O and dried.

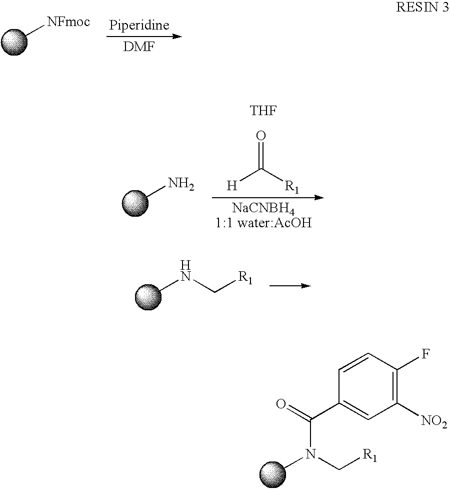

To Rink AM resin (Novabiochem, 0.4 mmol) was added a mixture of piperidine and DMF (6 mL, 50%) and the mixture was shaken for 45 min and filtered. The resin was thoroughly washed with DMF, i-PrOH, DCM (3× each), Et₂O and dried.

To the resin was added THF (8 mL), a mixture of aldehydes (0.4 mmol total) followed by a mixture of acetic acid and deionized water (50%, 800 μL). The mixture was shaken for 5 min and then NaCNBH₃ in THF was added (1M, 400 μL). The mixture was shaken for 3 hours at it and filtered (handle waste separately due to cyanoborohydride). The resin was washed with THF, H₂O, MeOH, THF, DCM (3× each), Et₂O and dried. To the resin was added DCM (5 mL), DIEA (315 μL, 1.8 mmol) and freshly prepared 4-fluoro-3-nitrobenzoyl chloride (1.5 mmol) in 3 mL DCM. The mixture was stirred overnight at rt. then filtered. The resin was washed with DCM, i-PrOH, DCM (3× each), Et₂O and dried.

### 4-(R)Amino-3-nitrobenzoic acid analogs

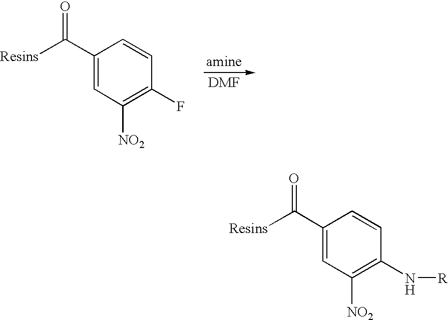

To resin (0.1 mmol) was added 5% DIEA in DMF (1 mL) and the amine (2.3 eq.) and the mixture was shaken overnight at rt. To vessels that contain hindered or slow reacting building blocks, a fast reacting building block from the same building block set was added (5 eq) and the mixture was shaken for 3 h at rt (capping). The resin was filtered off, washed with DMF, i-PrOH, DCM (3× each), Et₂O and dried.

### 4-(R)Amino-3-aminobenzoic acid analogs

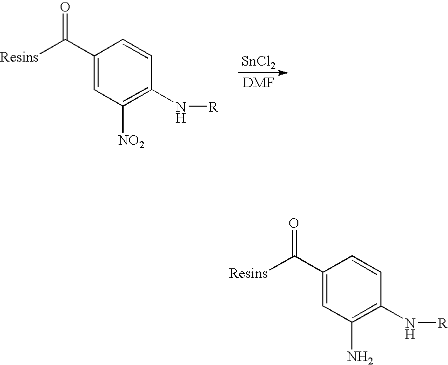

(Fresh anhydrous tin(II)-chloride and DMF are used for this step)

To resin (0.1 mmol) was added a 2M solution of tin(II)-chloride (fresh, anhydrous) in DMF (anhydrous, 1 mL) and the mixture was shaken overnight at it. The resin was filtered off, washed with DMF, i-PrOH, DCM (3× each), Et₂O and dried.

### Benzimidazole Analogs

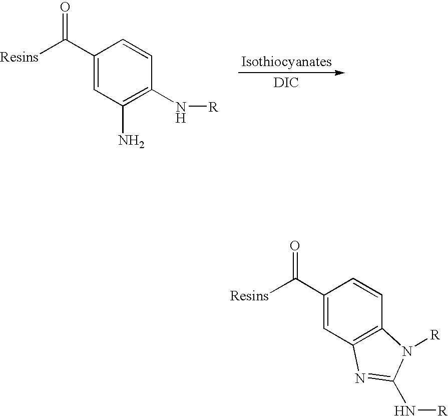

To resin (0.1 mmol) was added DMF (0.8 mL) and isothiocyanate (0.5 mmol) and the mixture was shaken for 1 day at rt. DIC (79 μL, 0.5 mmol) was injected and the mixture was shaken overnight at rt. The resin was filtered off, washed with DMF, i-PrOH, DCM (3× each), Et₂O and dried.

### Cleavage

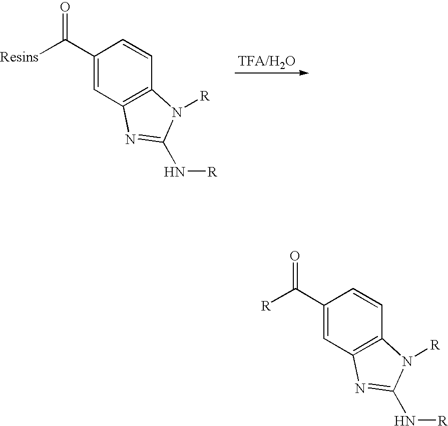

To the resin (0.1-0.2 mmol) was added TFA/H₂O (95:5, 1 mL) and the mixture was stirred for 1 h at rt. The resin was filtered and washed with acetonitrile (2×2 mL). The filtrate was concentrated in vacuo to yield the product.

### Method B

Similar reaction sequence and conditions to above protocol, however reaction was carried out in solution.

### Method C

Compounds were further modified after the cleavage step in Method A.

The synthesis of the compounds of paragraph (h) as described in U.S. application Ser. No. 60/858,244 is shown below.

### Synthesis of Compounds (h):

### Preparative Example 1

To a suspension of potassium carbonate (5.85 g, 1.5 equiv) and 4-(4,4,5,5-tetramethyl-1,3,2-dioxaborolan-2-yl)-1h-pyrazole (5.48 g, 1.0 equiv) in

NMP (50 mL) at rt was added SEMCl (5.2 mL, 1.05 equiv) dropwise (mildly exothermic). The resulting mixture was allowed to stir an additional 45 min at rt. The reaction was diluted with ethyl acetate, rinsed with water (2×), brine and dried (sodium sulfate). Filtration and concentration afforded the title compound that was used without purification. MH⁺=325.

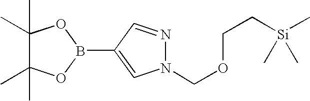

### Preparative Example 2

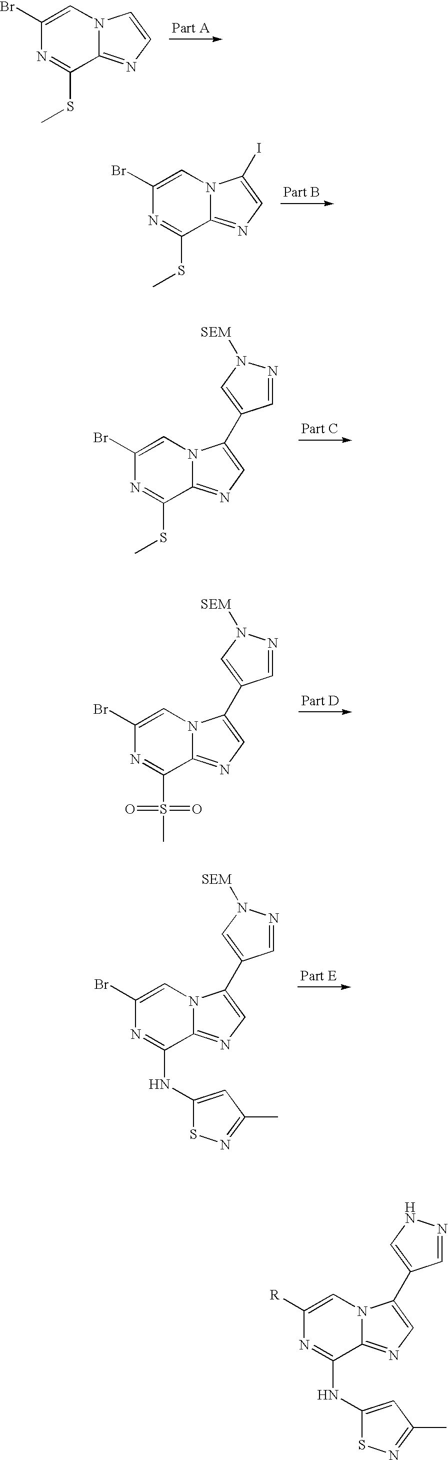

### Part A

To a solution of bromide (US2006/0106023) (2.00 g, 8.19 mmol) in DMF (50 mL) was added N-iodosuccinimide (1.84 g, 8.19 mmol). The reaction mixture was stirred at 60° C. for 16 h. The mixture was cooled to rt and concentrated. Purification by column chromatography (SiO₂, 40% ethyl acetate/hexanes) afforded compound 4 as a white solid 2.30 g (76%). ¹H-NMR (400 MHz, DMSO-d₆) δ 8.3 (s, 1H), 7.8 (s, 1H), 2.6 (s, 3H). MH⁺=371.

### Part B

A flask was charged with iodide from Part A (1.83 g, 1.00 equiv), boronate from Preparative Example 1 (2.08 g, 1.3 equiv), PdCl₂(dppf) (0.4 g, 0.1 equiv) and potassium phosphate monohydrate (3.4 g, 3.0 equiv). After purging the flask with argon, 1,4-dioxane (50 mL) and water (5 mL) were added and the resulting mixture was heated at 40° C. overnight (23 h). The reaction was cooled to rt. EtOAc was added to the reaction mixture and filtered through Celite. After concentration the residue was purified by column chromatography (silica gel, 25% EtOAc/hexane) to give the title compound (46%).

### Part C

To a solution of compound from Part B (1.02 g, 1.0 equiv) in DCM (10 mL) was added m-CPBA (1.1 g, 77%, 2.05 equiv) in one portion. The resulting mixture was stirred at rt for 30 min. The mixture was concentrated and then partitioned between EtOAc and water. The organic layer was washed with NaHCO₃ (sat. aq., twice), brine and dried (Na₂SO₄). After concentration, the crude product compound 6 was used in the next step directly without further purification.

### Part D

To a solution of 5-amino-3-methylisothiazole hydrochloride (0.135 g, 1.4 equiv) in DMSO (9 mL) at it was added NaH (0.11 g of 60% dispersion in oil, 3.0 equiv) in one portion. After ca. 10 min, compound from Part C (0.30 g, 1.00 equiv) was added in one portion. After 15 min at rt, the reaction was quenched with sat. aq. ammonium chloride and then extracted with ethyl acetate (×2). The combined organic layers were washed with water (×2), brine and, dried (sodium sulfate). The crude residue was purified by chromatography affording the title compound (0.18 g. 56%). MH⁺=506.

### Part E

A mixture of bromide from preparative example 3, Part D (30 mg, 0.059 mmol, 1 equiv), sodium methanethiolate (1.4 equiv), PdCl₂(dppf) (0.07 equiv), sodium t-butoxide (1.1 equiv) in 1,2-dimethoxyethane (1 mL) was stirred at 85° C. under Ar for 16 hours. The reaction mixture was cooled to room temperature, filtered through Celite and the filtrate concentrated. The residue was taken back up in ethyl acetate and washed with water, brine, dried over anhydrous sodium sulfate and concentrated to afford the crude residue. MH⁺=488. A solution of crude compound in THF (1 mL) was treated with 4N HCl in dioxane solution (1 mL) at 60° C. for 10 min. The solvent was removed and the residue was purified by Prep-LC., Conversion to a hydrochloric salt afforded the title compound.

The compounds in shown immediately below in Table A were prepared by essentially the same procedure as in preparative example 4.

TABLE A

| | | Exact | MS m/z | HPLC |
|---|---|---|---|---|
| Example | Compound | mass | (MH)⁺ | MS t_{R} |
| 4-1 | 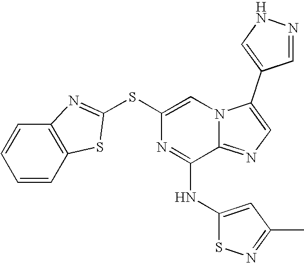 | 462 | 463 | 1.45 |
| 4-2 | 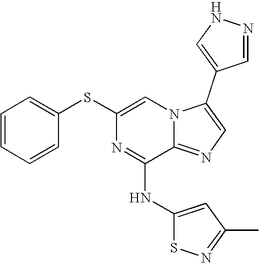 | 405 | 406 | 1.38 |
| 4-3 | 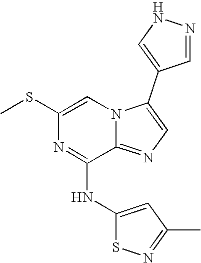 | 343 | 344 | 1.12 |
| 4.4 | 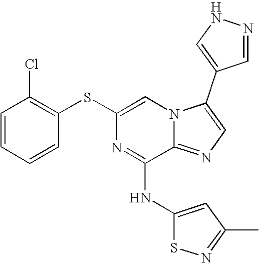 | 439.04 | 440.0 | 3.75 |
| 4.5 | 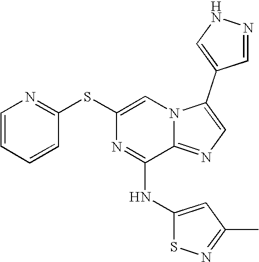 | 406.08 | 407.0 | 2.78 |
| 4-6 | 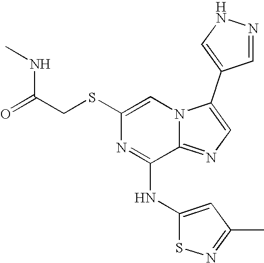 | 400.09 | 401.0 | 2.43 |
| 4-7 | 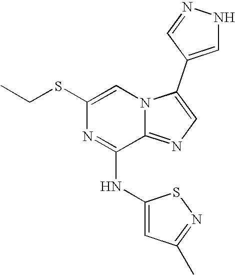 | 357.08 | 358.1 | 3.17 |
| 4-8 | 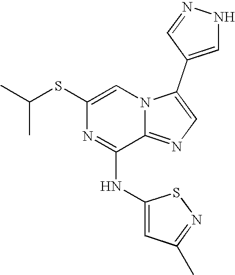 | 371.1 | 372.1 | 3.41 |
| 4.9 | 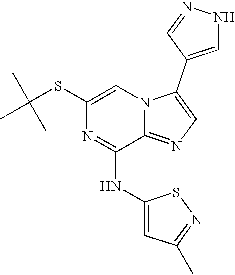 | 385.1 | 386.1 | 3.48 |
| 4-10 | 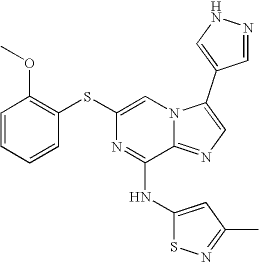 | 435.09 | 436.1 | 3.40 |
| 4-11 | 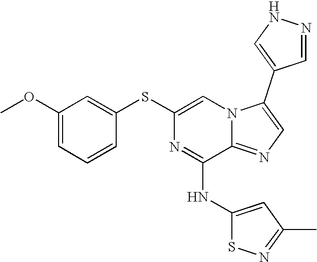 | 435.09 | 436.1 | 3.54 |
| 4-12 | 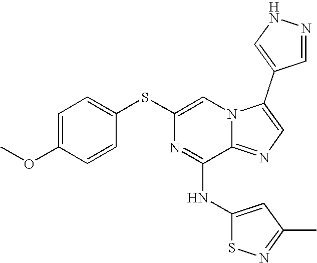 | 435.09 | 436.1 | 3.61 |
| 4-13 | 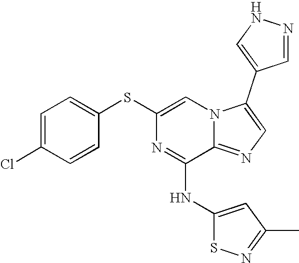 | 439.04 | 440.0 | 3.91 |
| 4-14 | 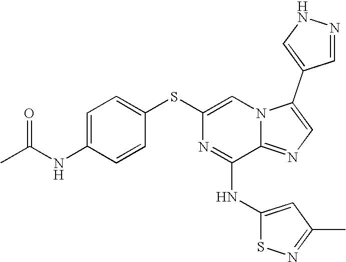 | 462.1 | 463.1 | 3.07 |
| 4-15 | 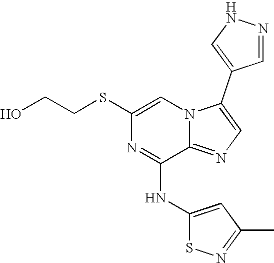 | 373.08 | 374.0 | 2.44 |
| 4-16 | 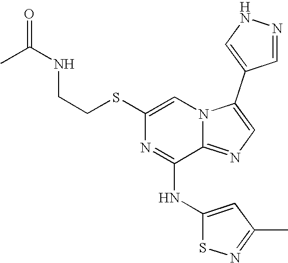 | 414.1 | 415.1 | 2.53 |
| 4-17 | 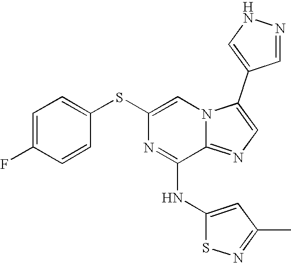 | 423.07 | 424.1 | 3.63 |
| 4-18 | 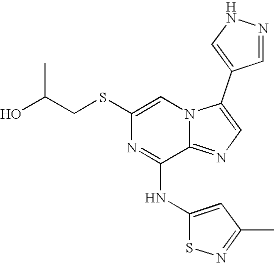 | 387.09 | 388.1 | 2.64 |
| 4-19 | 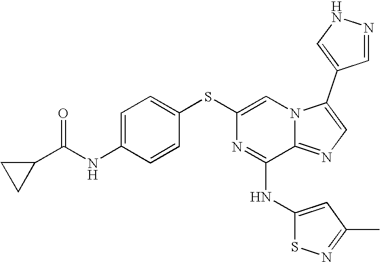 | 488.12 | 489.1 | 3.44 |
| 4-20 | 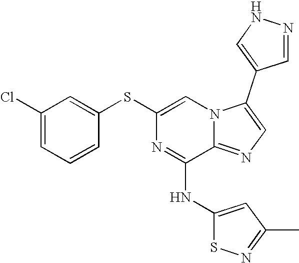 | 439.04 | 440.0 | 3.88 |
| 4-21 | 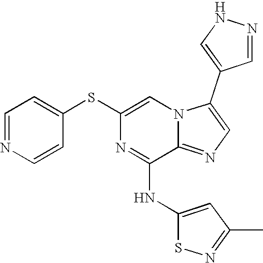 | 406.08 | 407.0 | 2.36 |
| 4-22 | 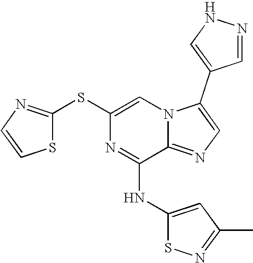 | 412.03 | 413.0 | 2.89 |
| 4-23 | 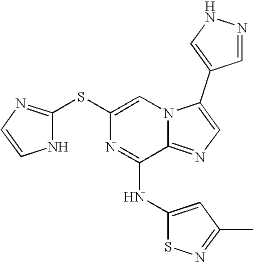 | 395.07 | 396.1 | 2.07 |
| 4-24 | 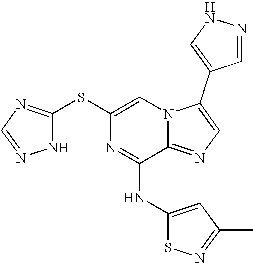 | 396.07 | 397.1 | 2.28 |
| 4-25 | 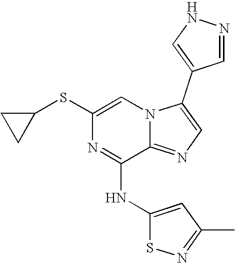 | 369.1 | 370.1 | |
| 4-26 | 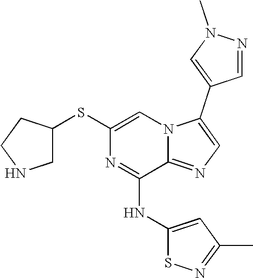 | 412.1 | 413.1 | 2.5 |
| 4-27 | 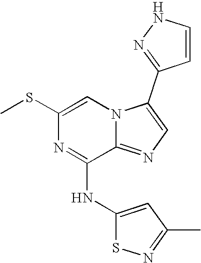 | 343.1 | 344.1 | 1.38 |
| 4-28 | 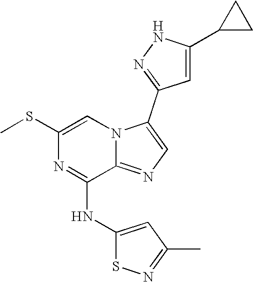 | 383.1 | 384.1 | 1.48 |
| 4-29 | 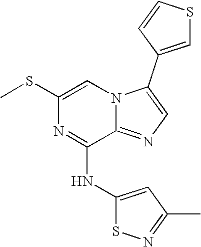 | 395.0 | 396.0 | 5.63 |
| 4-30 | 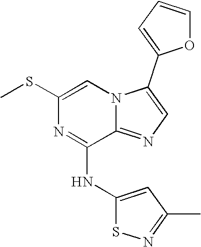 | 343.1 | 344.1 | 5.5 |
| 4-31 | 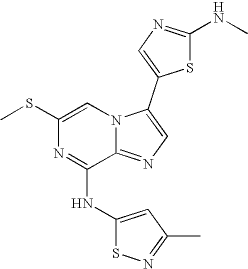 | 389.1 | 390.1 | 3.8 |
| 4-32 | 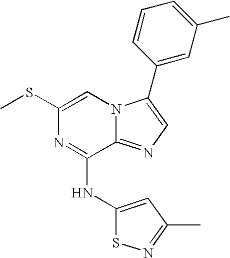 | 367.1 | 367.1 | 6.2 |
| 4-33 | 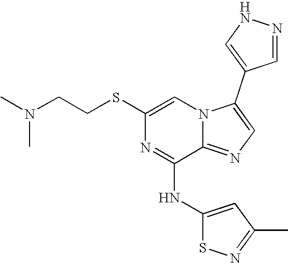 | 400.1 | 401.1 | 0.85 |
| 4-34 | 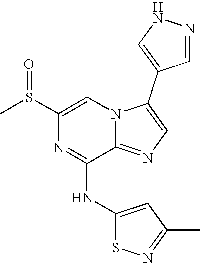 | 359.1 | 360.1 | 2.6 |
| 4-35 | 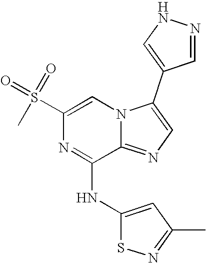 | 375.1 | 376.1 | 2.6 |

### Synthesis of Compounds of (i):

The synthesis of the compounds of paragraph (i) as described in U.S. applications, Ser. No. 60/943,999 filed Jun. 14, 2007 and Ser. No. 60/987,932, filed Nov. 14, 2007 is shown below.

### Example 1

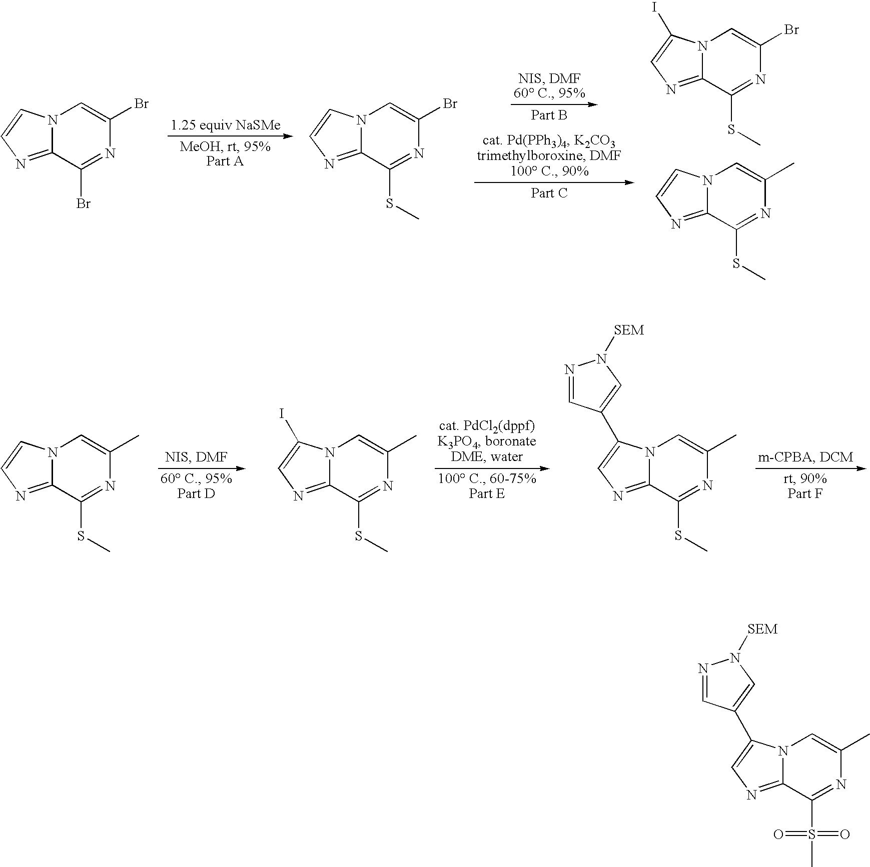

Part A: Prepared according to US20060106023 (A1).

Part B: To a solution of compound from Example 1, Part A (2.00 g, 8.19 mmol) in DMF (50 mL) was added N-iodosuccinimide (1.84 g, 8.19 mmol). The reaction mixture was stirred at 60 C for 16 hours. The mixture was cooled to 25 C and concentrated. The residue was dissolved in DCM with a small amount of methanol and then loaded on the column. Purification by column chromatography (SiO₂, 40% ethyl acetate/hexanes) afforded compound 4 as a white solid 2.30 g (76%). ¹H-NMR (400 MHz, DMSO-d₆) δ 8.3 (s, 1H), 7.8 (s, 1H), 2.6 (s, 3H). HPLC-MS t_{R}=1.87 Min (UV₂₅₄ₙₘ). Mass calculated for formula C₇H₅BrIN3S 370.01, observed LC/MS m/z 370.9 (M+H).

Part C: A suspension of bromide from Part B (45.6 g), Pd(PPh3)₄ (10.8 g), potassium carbonate (77.4 g), trimethylboroxine (46.9 g) and potassium carbonate (77.4 g) in DMF (410 mL) was heated overnight under nitrogen at 105 C. After cooling, the mixture was diluted with ethyl acetate (1 L), washed with brine (2×500 mL), dried (magnesium sulfate), filtered, concentrated and purified by chromatography on silica gel. The title compound was obtained as a pale yellow solid (21.4 g, 64%).

Part D: To a DMF (400 mL) solution of compound from Example 1, Part C (21.8 g) was added N-iodosuccinimide (26.9 g) and the resulting mixture was heated overnight at 60 C. The mixture was concentrated and water (400 mL) was added. After stirring 1 hr at rt, saturated sodium carbonate was added (250 mL) and subsequently stirred an additional 30 min at rt. The mixture was filtered, washed with water, methanol (100 mL) and the filter cake was dried overnight under vacuum. A brown solid was obtained (31.4 g, 87%).

Part E: A flask was charged with iodide from Part D (1.00 equiv), Bpin-compound 5a (1.3 equiv), PdCl2(dppf) (0.1 equiv) and potassium phosphate monohydrate (3.0 equiv). After purging the flask with argon, 1,4-dioxane (50 mL) and water (5) were added and the resulting mixture was heated at 80 C overnight (23 h). The reaction was cooled to room temperature. EtOAc was added to the reaction mixture and filtered through Celite. After concentration the residue was purified by column chromatography (silica gel, 25% EtOAc/hexane) to give the title compound.

Part F: To a solution of compound from Example 1, Part E (1.0 equiv) in DCM (10 mL) was added m-CPBA (2.05 equiv) in one portion. The resulting mixture was stirred at room temperature for 30 min. The mixture was concentrated and then partitioned between EtOAc and water. The organic layer was washed with NaHCO₃ (sat. aq., twice), brine and dried (Na₂SO₄). After concentration, the title compound was obtained and used in the next step directly without further purification.

### Example 2

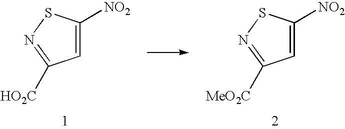

To a solution of 1¹ (1.04 g, 5.98 mmol) in 20 mL of DMF, was added K₂CO₃ (2.48 g, 17.9 mmol) and MeI (1.27 g, 8.96 mmol). The reaction was stirred at room temperature overnight. It was diluted with 200 mL of 50% EtOAc/hexanes and washed with water (200 mL) and brine (100 mL). The organic was concentrated. To the residue was added 20 mL of hexanes. The solid was collected by filtration to give 2. The filtrate was concentrated and purified by column eluting with 25% EtOAc/hexanes to give additional amount of 2. The combined yield of 2 is 1.05 g. ¹H NMR (400 MHz, CDCl₃) δ 8.37 (s, 1H), 4.05 (s, 3H).

### Example 3

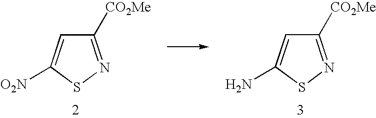

To a solution of 2 (260 mg, 1.38 mmol) in 6 mL of AcOH was added iron powder (774 mg, 13.8 mmol). The reaction mixture was heated at 70-75° C. for 12 min. The mixture was cooled to room temperature and then added 20 mL of MeOH. The resulting mixture was filtered through celite (the celite was rinsed with additional amount MeOH). The filtrate was concentrated to remove most of AcOH. To the residue was added 15 mL of 20% MeOH/CH₂Cl₂ followed by 20 mL of saturated aqueous NaHCO₃. The mixture was stirred until it stops bobbling. The mixture was extracted by EtOAc (60 mL×2), dried over Na₂SO₄, and then concentrated. To the residue was added 5 mL of ether followed by 5 mL of hexanes.

The solid was collected by filtration to give 160 mg of crude 3 which contains a little acylated amine but pure enough for the sulfone displacement reaction. The filtrate was purified by column with 20% of AcOEt/CH₂Cl₂ to give additional 30 mg of 3. ¹H NMR (400 MHz, CDCl₃) δ 6.85 (s, 1H), 4.61 (brs, 2H), 3.92 (s, 3H).

### Example 4

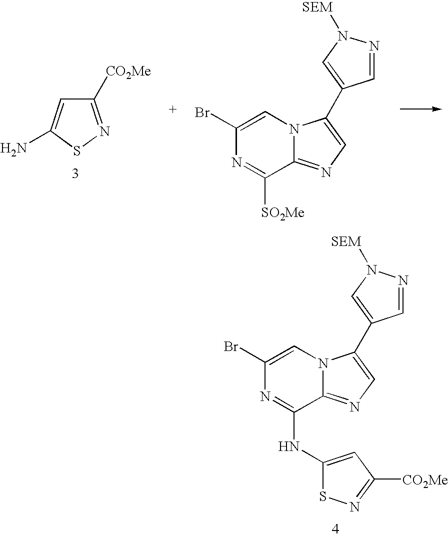

To a solution of compound 3 (89 mg, 0.56 mmol) and 6-bromo-8-methanesulfonyl-3-[1-(2-trimethylsilanyl-ethoxymethyl)-1H-pyrazol-4-yl]-imidazo[1,2-a]pyrazine (258 mg, 0.55 mmol) in 2 mL of DMF, was added NaH (60% dispersion in oil, 44 mg, 1.1 mmol). The reaction was stirred at room temperature for 15 min. It was quenched with 5 mL of saturated aqueous NH₄Cl and diluted with 30 mL of water. The solid was collected by filtration, washed with water and MeOH. It was dried under vacuum to give 255 mg of compound 4. ¹H NMR (400 MHz, DMSO-d₆) δ 8.85 (s, 1H), 8.22 (s, 1H), 8.15 (s, 1H), 7.96 (s, 1H), 7.62 (s, 1H), 5.50 (s, 2H), 3.85 (s, 3H), 3.60 (t, 2H), 1.83 (t, 2H), 0.00 (s, 9H).

### Example 5

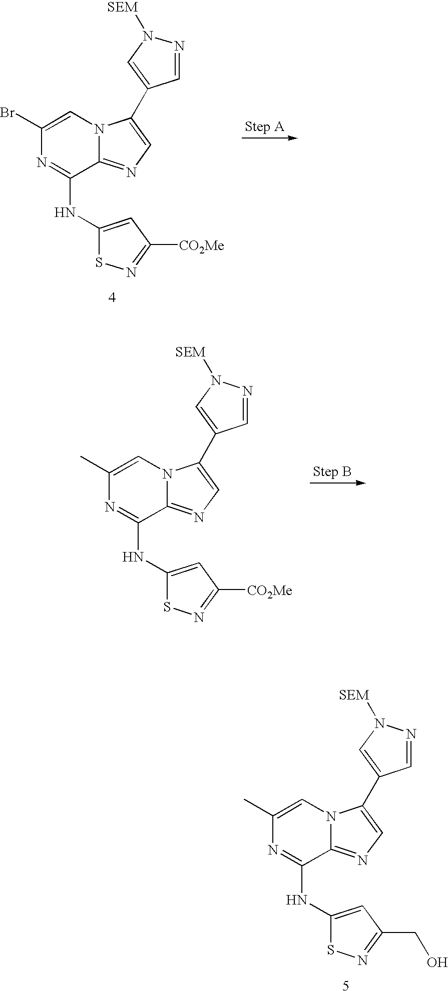

Step A: To a solution of 4 (76 mg, 0.14 mmol) in 6 mL of THF was added Pd(PPh₃)₄ (16 mg, 0.014 mmol) and 0.35 mL of MeZnCl (2 M solution in THF, 0.69 mmol). The reaction was stirred at 80° C. for 20 min. It was cooled to room temperature and quenched by adding 0.5 mL of MeOH. It was diluted with 30 mL of CH₂Cl₂ and washed with 20 mL of 0.5 N aqueous HCl solution. The solvent was removed under vacuum. The residue was purified by flash chromatography eluting with 5% MeOH/CH₂Cl₂ to give 50 mg of 5-{6-methyl-3-[1-(2-trimethylsilanyl-ethoxymethyl)-1H-pyrazol-4-yl]-imidazo[1,2-a]pyrazin-8-ylamino}-isothiazole-3-carboxylic acid methyl ester contaminated by a small amount of triphenylphosphine oxide.

Step B: The above crude material was dissolved in 5 mL of THF. To the solution was added 0.5 mL of LiBHEt₃ (1 M solution in THF). The reaction was stirred at room temperature for 30 min. It was quenched by adding 5 mL of saturated aqueous NH₄Cl solution. The mixture was extracted by 30 mL of CH₂Cl₂. The organic was concentrated and purified by flash chromatography eluting with 5% MeOH/CH₂Cl₂ to give 25 mg of compound 5. NMR (400 MHz, CDCl₃) δ 7.90 (s, 1H), 7.82 (s, 1H), 7.60 (s, 1H), 7.48 (s, 1H), 6.90 (s, 1H), 5.55 (s, 2H), 4.75 (brs, 2H), 3.65 (t, 2H), 2.50 (s, 3H), 1.00 (t, 2H), 0.00 (s, 9H).

### Example 6

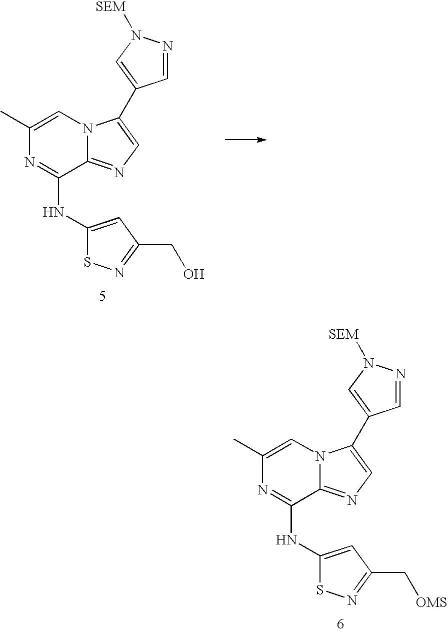

To a solution of compound 5 (200 mg, 0.438 mmol) in 20 mL of THF, was added triethylamine (0.21 mL, 1.5 mmol) and methanesulfonylchloride (0.10 mL, 1.3 mmol). The reaction was stirred at room temperature for 30 min. It was quenched by adding 1 mL of MeOH. The solution was diluted by 30 mL of CH₂Cl₂, washed consecutively with 15 mL of 2

N aqueous HCl, water, and brine. The solvent was removed under vacuum to give 230 mg of crude compound 6 which was used in further transformations without further purification.

### Example 7

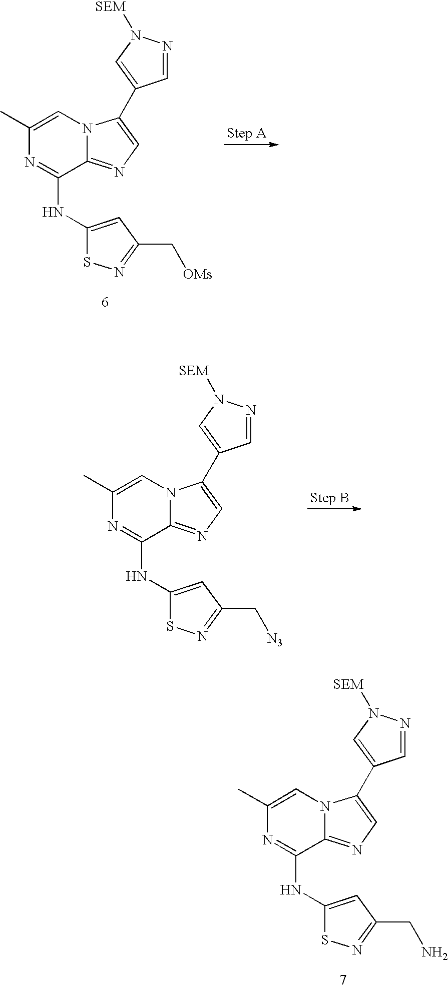

Step A: Mixture of compound 6 (17 mg, 0.032 mmol) and sodium azide (15 mg, 0.23 mmol) in 1 mL of DMF was heated at 70° C. for 3 h. It was cooled to room temperature and added 10 mL of water. The resulting solid was collected by filtration and purified by flash chromatography eluting with 5% MeOH/CH₂Cl₂ to give 12 mg of (3-azidomethyl-isothiazol-5-yl)-{6-methyl-3-[1-(2-trimethylsilanyl-ethoxymethyl)-1H-pyrazol-4-yl]-imidazo[1,2-a]pyrazin-8-yl}-amine.

Step B: The above material was dissolved in 3 mL of MeOH. To the solution was added 15 mg of 10% wt. Pd/C. The mixture was stirred under H₂ (1 atm) for 1 h. It was filtered through celite. The filtrate was concentrated under vacuum to give 12 mg of compound 7. NMR (400 MHz, CDCl₃) δ 7.88 (s, 1H), 7.80 (s, 1H), 7.60 (s, 1H), 7.47 (s, 1H), 6.86 (s, 1H), 5.55 (s, 2H), 4.00 (brs, 2H), 3.65 (t, 2H), 2.50 (s, 3H), 1.00 (t, 2H), 0.00 (s, 9H).

### Example 8

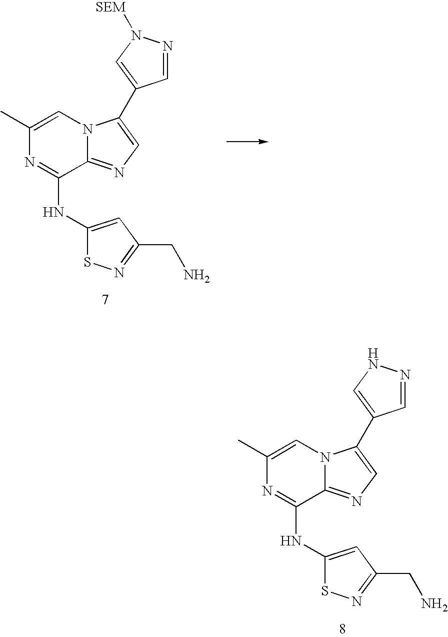

To a solution of compound 7 (12 mg, 0.026 mmol) in 2 mL of THF heated at 70° C., was added 0.5 mL of 4 N HCl in dioxane. To the resulting mixture was added MeOH until it became homogeneous. The reaction was stirred at 70° C. for 1 h and then cooled to room temperature. The solid was collected by filtration and washed with ether to give 9 mg of compound 8 as its HCl salt form. NMR (400 MHz, CD₃OD) δ 8.23 (s, 1H), 8.20 (s, 2H), 8.03 (s, 1H), 7.20 (s, 1H), 4.22 (s, 2H), 2.59 (s, 3H). HPLC-MS t_{R}=1.82 min (UV₂₅₄ₙₘ). Mass calculated for formula C₁₄H₁₄N₈S 326.1; observed MH⁺ (LCMS) 327.2 (m/z).

### Example 9

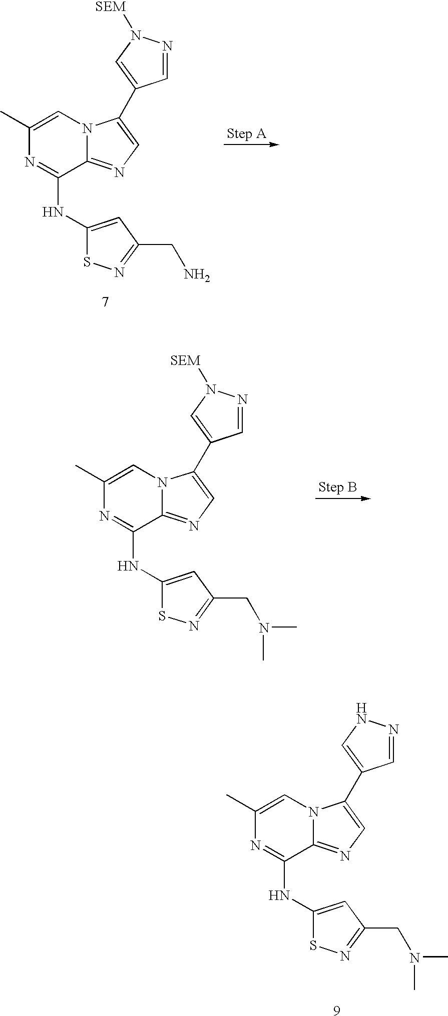

Step A: To a solution of compound 7 (9 mg, 0.02 mmol) in 1 mL of MeOH/CH₂Cl₂ (1:1), was added formaldehyde (40% wt. in water, 6 mg, 0.2 mmol). It was stirred at room temperature for 15 min when NaBH₄ (16 mg, 0.4 mmol) was added in two portions. The mixture was purified by flash chromatography eluting with NH4Cl (aq.)/MeOH/CH₂Cl₂ (1:5:190) to give 5 mg of (3-dimethylaminomethyl-isothiazol-5-yl)-{6-methyl-3-[1-(2-trimethylsilanyl-ethoxymethyl)-1H-pyrazol-4-yl]-imidazo[1,2-a]pyrazin-8-yl}-amine.

Step B: The above material was then dissolved in 2 mL of THF. The resulting solution was heated at 70° C. when 0.5 mL of 4N HCl in dioxane was added. To the resulting mixture was added 1 mL of MeOH. The reaction was stirred at 70° C. for 1 h and then cooled to room temperature. Most of the solvent was removed under vacuum. To the residue was added 5 mL of ether. The solid was collected by filtration, and washed with ether to give 5 mg of compound 9 as its HCl salt form. NMR (400 MHz, CD₃OD) δ 8.21 (s, 1H), 8.18 (s, 2H), 8.08 (s, 1H), 7.30 (s, 1H), 4.42 (s, 2H), 2.95 (s, 6H), 2.59 (s, 3H). HPLC-MS t_{R}=2.04 min (UV₂₅₄ₙₘ). Mass calculated for formula C₁₆H₁₈N₈S 354.1; observed MH⁺ (LCMS) 355.2 (m/z).

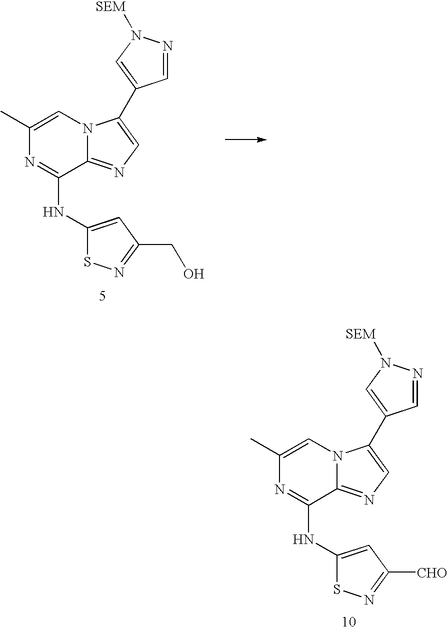

### Example 10

To a solution of compound 5 (2.50 g, 5.47 mmol) in 100 mL of THF, was added 0.3 mL of water followed by Dess-Martin periodinane (6.96 g, 16.4 mmol). The reaction was stirred at room temperature for 30 min. The solid was filtered off. The filtrate was diluted with 200 mL of CH₂Cl₂, and washed with 100 mL of saturated aqueous NH₄Cl solution. The organic was' dried over anhydrous Na₂SO₄ and then concentrated. To the residue was added 30 mL of acetonitrile. The solid was collected by filtration to give 2.05 g of compound 10. NMR (400 MHz, DMSO-d₆) δ 12.38 (s, 1H), 9.84 (s, 1H), 8.60 (s, 1H), 8.11 (s, 1H), 7.96 (s, 1H), 7.91 (s, 1H), 7.55 (s, 1H), 5.50 (s, 2H), 3.60 (t, 2H), 2.45 (s, 3H), 1.83 (t, 2H), 0.00 (s, 9H).

### Example 11

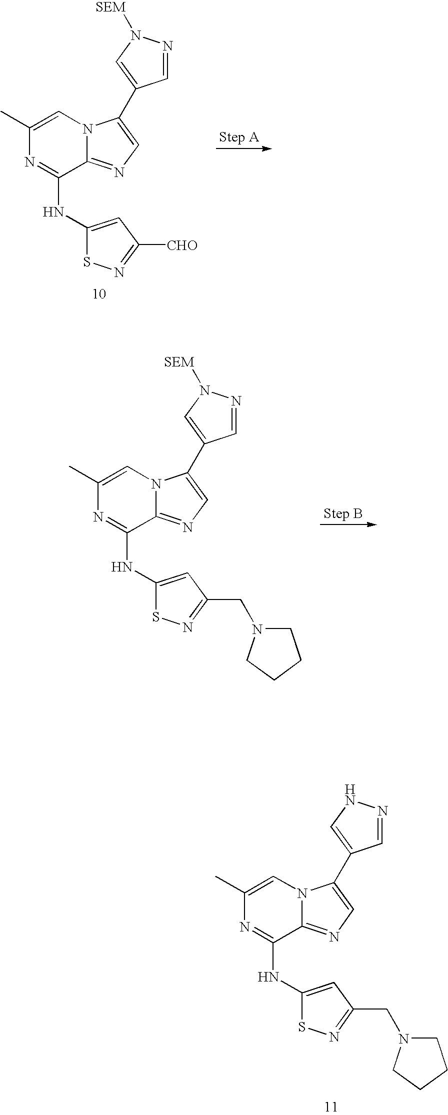

Step A: A solution of compound 10 (100 mg, 0.220 mmol) and pyrrolidine (156 mg, 2.20 mmol) in 14 mL of CH₂Cl₂ was stirred at room temperature for 20 min. To the solution was added two drops of acetic acid, followed by NaBH₄ (67 mg, 1.8 mmol). The resulting mixture was stirred at room temperature for 5 min when 3 mL of MeOH was added. The stirring was continued for additional 20 min. The reaction was quenched by adding 15 mL of saturated aqueous NaHCO₃ solution. After diluted with 20 mL of CH₂Cl₂, the organic was isolated. The solvent was removed under vacuum. The residue was purified by flash chromatography eluting with NH₄Cl(aq.)/MeOH/CH₂Cl₂ (1:10:190) to give 98 mg of {6-methyl-3-[1-(2-trimethylsilanyl-ethoxymethyl)-1H-pyrazol-4-yl]-imidazo[1,2-a]pyrazin-8-yl}-(3-pyrrolidin-1-ylmethyl-isothiazol-5-yl)-amine. NMR (400 MHz, CDCl₃) δ 7.88 (s, 1H), 7.81 (s, 1H), 7.60 (s, 1H), 7.48 (s, 1H), 6.96 (s, 1H), 5.55 (s, 2H), 3.80 (s, 2H), 3.65 (t, 2H), 2.70 (brs, 4H), 2.50 (s, 3H), 1.85 (brs, 4H), 0.96 (t, 2H), 0.00 (s, 9H).

Step B: To a solution of {6-methyl-3-[1-(2-trimethylsilanyl-ethoxymethyl)-1H-pyrazol-4-yl]-imidazo[1,2-a]pyrazin-8-yl}-(3-pyrrolidin-1-ylmethyl-isothiazol-5-yl)-amine (98 mg, 0.19 mmol) in 8 mL of THF heated at 70° C., was added 2 mL of 4 N HCl in dioxane. To the resulting mixture was added MeOH until it became homogeneous. The reaction was stirred at 70° C. for 1 h and then cooled to room temperature. To the mixture was added 3 mL of ether. The solid was collected by filtration and washed with ether to give 79 mg of compound 11 as its HCl salt form. NMR (400 MHz, CD₃OD) δ 8.18 (s, 2H), 8.13 (s, 1H), 8.00 (s, 1H), 7.22 (s, 1H), 4.50 (s, 2H), 3.62-3.68 (m, 2H), 3.06-3.15 (m, 2H), 2.58 (s, 3H), 1.95-2.22 (m, 4H). HPLC-MS t_{R}=2.03 min (UV₂₅₄ₙₘ). Mass calculated for formula C₁₈H₂₀N₈S 380.2; observed MH⁺ (LCMS) 381.2 (m/z).

### Example 12

By essentially the same procedure set forth in Example 11, only replacing pyrrolidine with other respective aliphatic amines in step A, compounds shown in column 2 of Table 1 shown immediately below were prepared.

TABLE 1

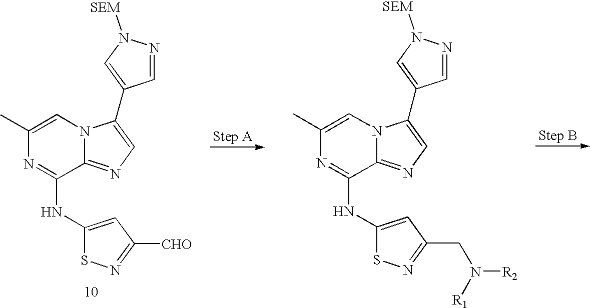

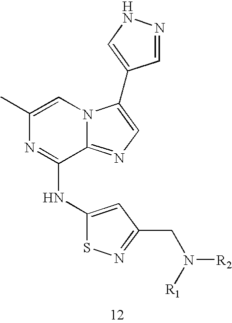

| | | | LCMS | |
|---|---|---|---|---|
| | | | MH⁺ | HPLC |
| Example | Column 2 | MW | m/z | MS t_{R} |
| 12-1 | 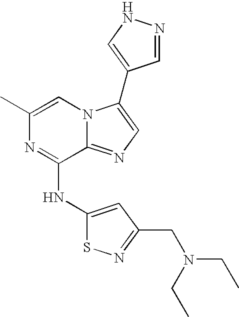 | 382.5 | 383.2 | 2.15 |
| 12-2 | 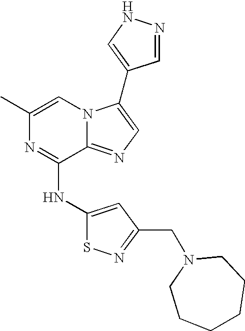 | 408.5 | 409.2 | 2.31 |
| 12-3 | 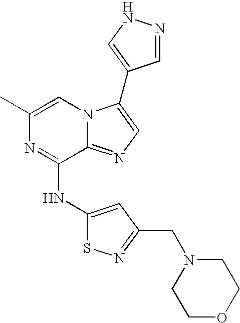 | 396.5 | 229.2 | 1.49 |
| 12-4 | 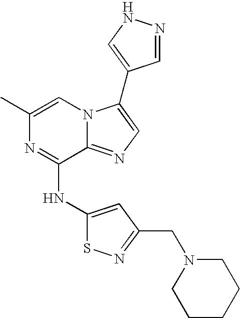 | 394.5 | 395.2 | 2.18 |
| 12-5 | 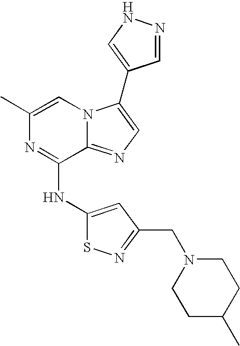 | 408.5 | 409.2 | 2.26 |
| 12-6 | 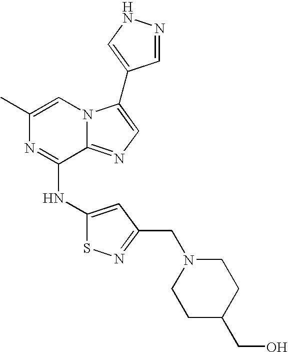 | 424.5 | 425.2 | 1.95 |
| 12-7 | 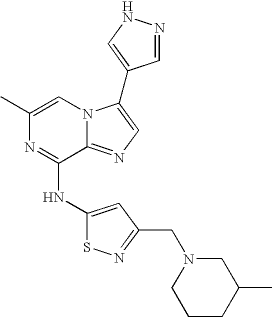 | 408.5 | 409.2 | 2.35 |
| 12-8 | 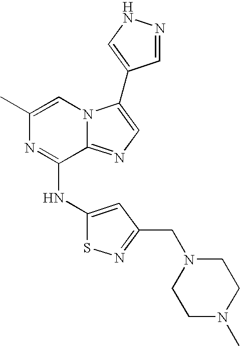 | 409.5 | 410.2 | 1.76 |
| 12-9 | 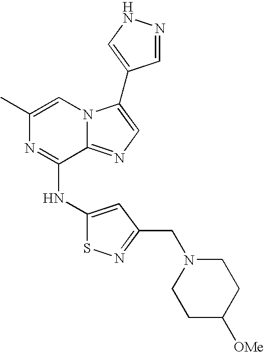 | 424.5 | 425.2 | 2.18 |
| 12-10 | 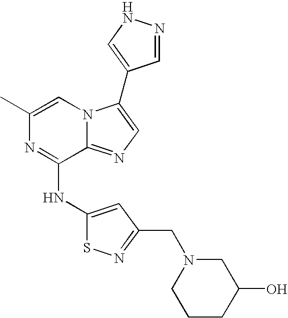 | 410.5 | 411.2 | 2.04 |
| 12-11 | 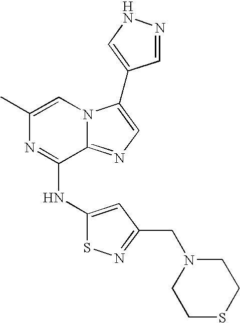 | 412.5 | 413.2 | 2.23 |
| 12-12 | 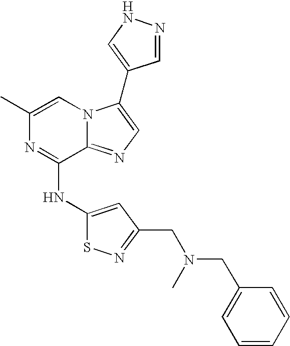 | 430.5 | 431.2 | 2.35 |
| 12-13 | 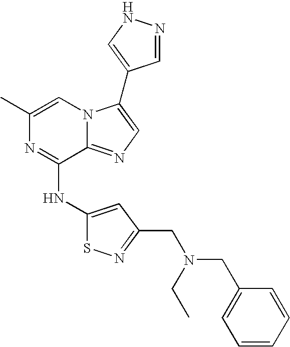 | 444.6 | 445.2 | 2.40 |
| 12-14 | 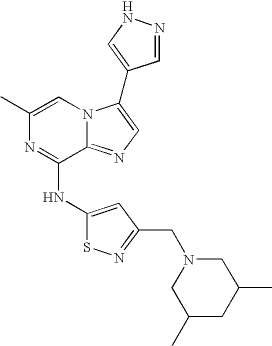 | 422.5 | 423.2 | 2.50 |
| 12-15 | 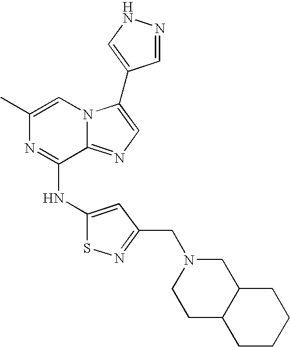 | 448.6 | 449.2 | 2.69 |
| 12-16 | 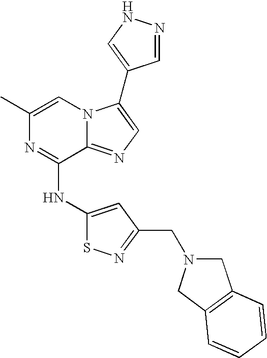 | 428.5 | 429.2 | 2.34 |
| 12-17 | 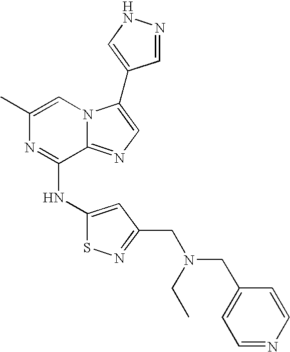 | 445.5 | 446.2 | 1.76 |
| 12-18 | 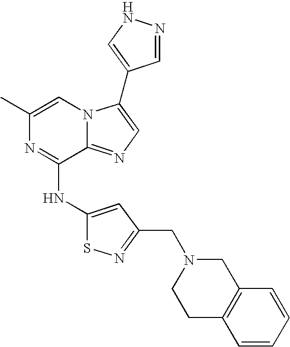 | 442.5 | 443.2 | 2.39 |
| 12-19 | 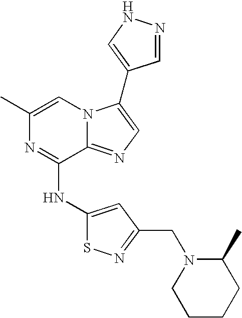 | 408.5 | 409.2 | 2.15 |
| 12-20 | 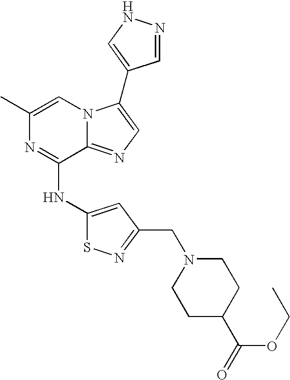 | 466.6 | 467.3 | 2.23 |
| 12-21 | 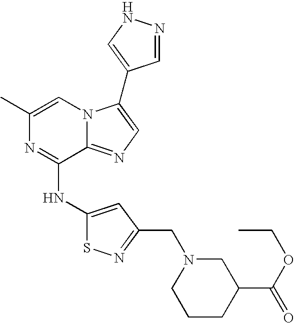 | 466.6 | 467.3 | 2.24 |
| 12-22 | 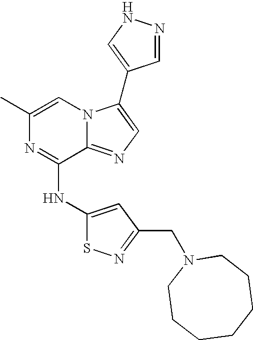 | 422.5 | 423.2 | 2.41 |
| 12-23 | 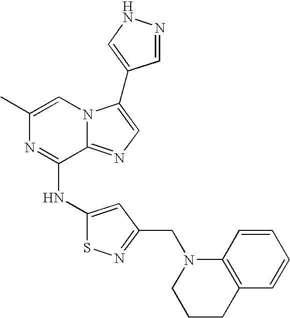 | 442.5 | 443.2 | 3.51 |
| 12-24 | 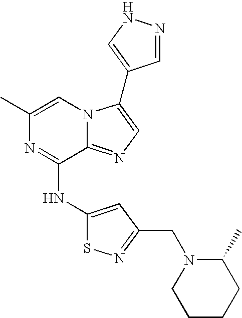 | 408.5 | 409.2 | 2.32 |
| 12-25 | 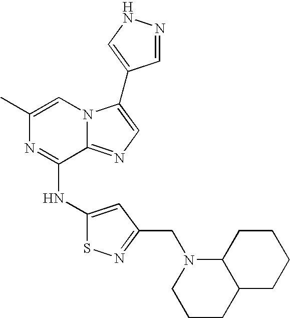 | 448.5 | 449.2 | 2.65 |
| 12-26 | 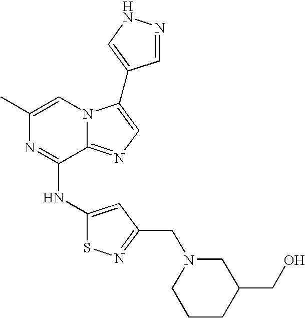 | 424.5 | 425.2 | 2.07 |
| 12-27 | 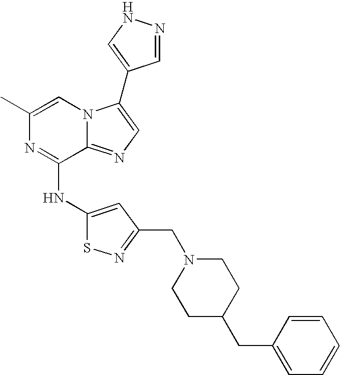 | 484.6 | 485.3 | 2.79 |
| 12-28 | 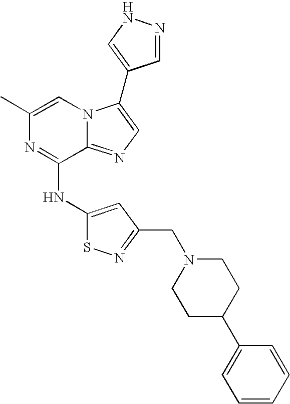 | 470.6 | 471.3 | 2.60 |
| 12-29 | 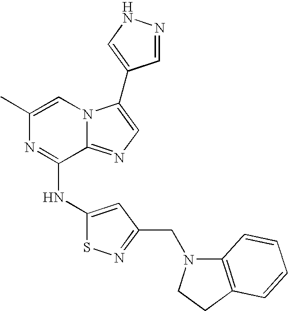 | 428.5 | 429.2 | 3.27 |
| 12-30 | 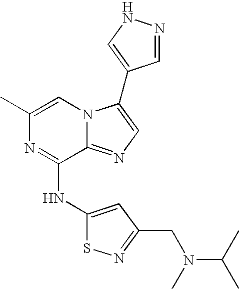 | 382.17 | 393.17 | 1.97 |
| 12-31 | 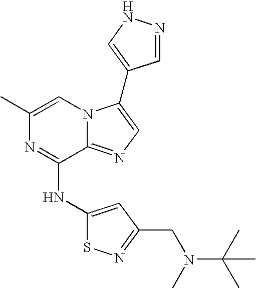 | 396.18 | 397.18 | 2.01 |
| 12-32 | 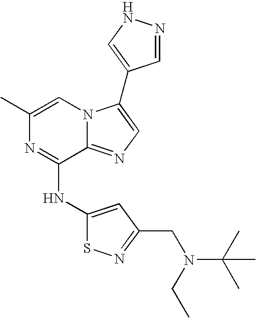 | 410.20 | 411.20 | 2.04 |
| 12-33 | 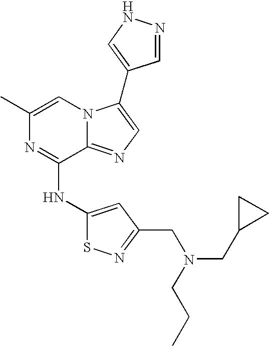 | 422.20 | 423.20 | 2.52 |
| 12-34 | 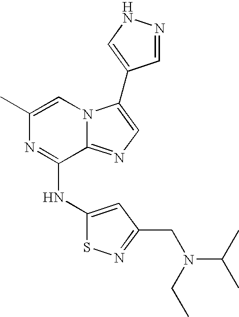 | 396.18 | 397.18 | 2.25 |
| 12-35 | 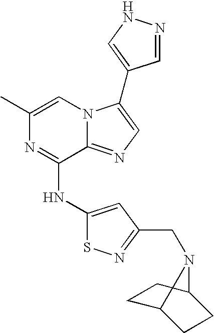 | 406.17 | 407.17 | 2.24 |
| 12-36 | 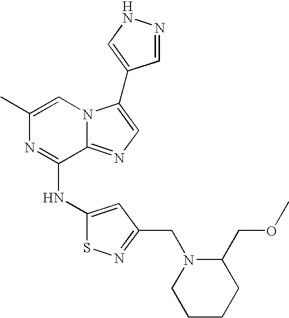 | 438.20 | 439.20 | 2.36 |
| 12-37 | 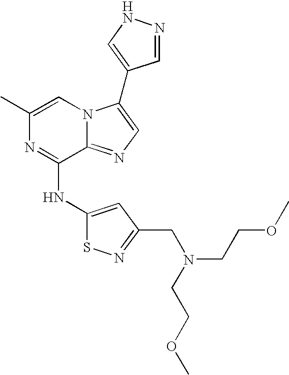 | 442.19 | 443.19 | 2.30 |
| 12-38 | 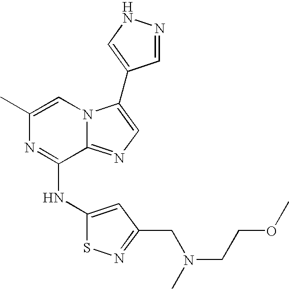 | 398.16 | 399.16 | 2.12 |

### Example 13

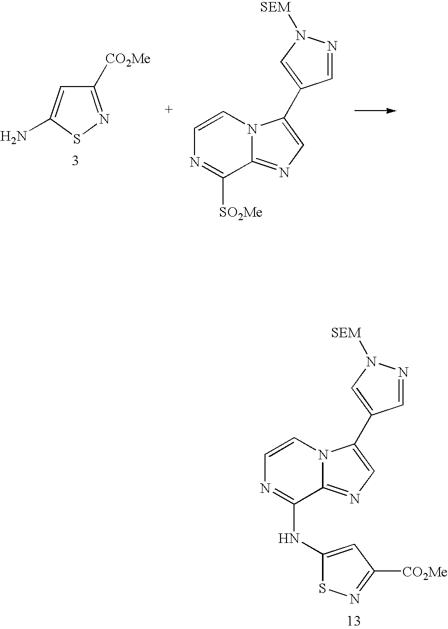

By essentially the same procedure set forth in Example 4, only replacing 6-bromo-8-methanesulfonyl-3-[1-(2-trimethylsilanyl-ethoxymethyl)-1H-pyrazol-4-yl]-imidazo[1,2-a]pyrazine with 8-methanesulfonyl-3-[1-(2-trimethylsilanyl-ethoxymethyl)-1H-pyrazol-4-yl]-imidazo[1,2-a]pyrazine, compound 13 was prepared. NMR (400 MHz, DMSO-d₆) δ 8.66 (s, 1H), 8.22 (d, 1H), 8.21 (s, 1H), 8.03 (s, 1H), 7.82 (d, 1H), 5.58 (s, 2H), 3.96 (s, 3H), 3.69 (t, 2H), 3.40 (s, 1H), 0.95 (t, 2H), 0.02 (s, 9H).

### Example 14

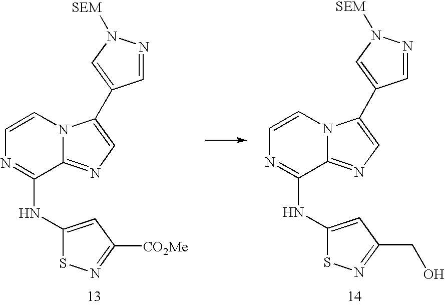

To a solution of compound 13 (830 mg, 1.76 mmol) in 50 mL of CH₂Cl₂ stirred at 0° C., was added 7.05 mL of LiBHEt₃ (1 M solution in THF). The reaction was stirred at room temperature for 10 min. It was quenched by adding saturated aqueous NH₄Cl solution. The organic was separated and washed with saturated aqueous NaHCO₃ solution. The solvent was removed under vacuum. To the residue was added 10 mL of MeOH. The solid was collected by filtration to give 530 mg of compound 14. NMR (400 MHz, CD₃OD) δ 8.38 (s, 1H), 7.98 (s, 1H), 7.97 (d, 2H), 7.78 (s, 1H), 7.69 (d, 2H), 7.15 (s, 1H), 5.55 (s, 2H), 4.62 (s, 2H), 3.66 (t, 2H), 0.92 (t, 2H), 0.00 (s, 9H).

### Example 15

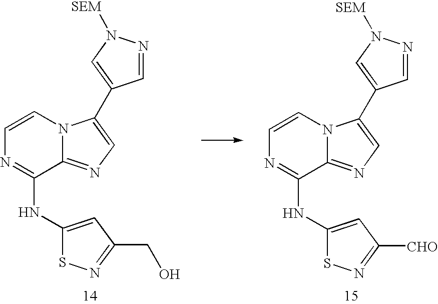

To a solution of compound 14 (258 mg, 0.582 mmol) in 20 mL of THF, was added 0.05 mL of water followed by Dess-Martin periodinane (740 mg, 1.75 mmol). The reaction was stirred at room temperature for 1.5 h. The solid was filtered off. The filtrate was diluted with 100 mL of CH₂Cl₂, and washed with water and brine. The solvent was removed under vacuum, the residue was purified by flash chromatography eluting with 5% of MeOH/CH₂Cl₂ to give 230 mg of compound 15.

### Example 16

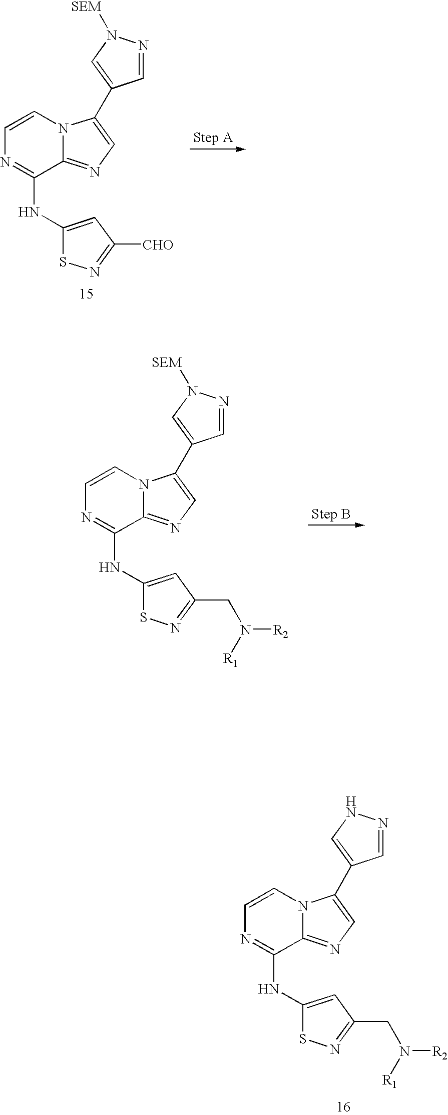

By essentially the same procedure set forth in Example 11, compounds shown in column 2 of Table 2 (shown immediately below) were prepared by replacing compound 10 with compound 15, and replacing pyrrolidine with other respective aliphatic amines in step A.

TABLE 2

| | | | LCMS | |
|---|---|---|---|---|
| | | | MH⁺ | HPLC |
| Example | Column 2 | MW | m/z | MS t_{R} |
| 16-1 | 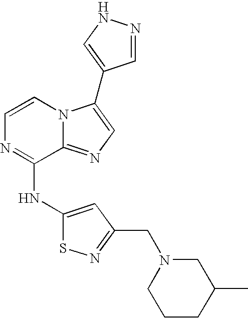 | 394.5 | 395.2 | 2.02 |
| 16-2 | 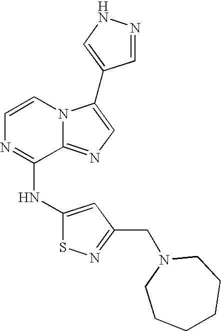 | 394.5 | 395.2 | 2.13 |
| 16-3 | 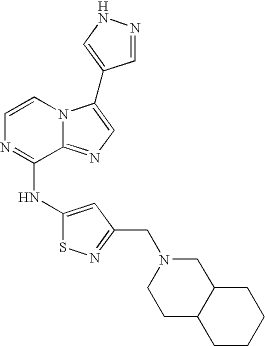 | 434.6 | 435.2 | 2.50 |
| 16-4 | 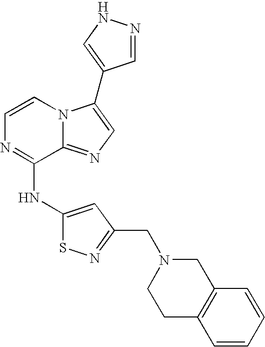 | 428.5 | 429.2 | 2.22 |
| 16-5 | 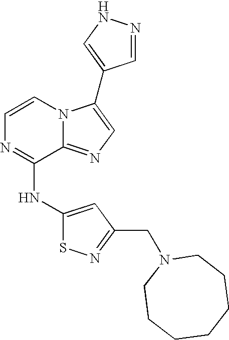 | 408.5 | 409.2 | 2.18 |
| 16-6 | 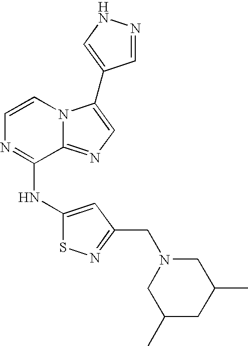 | 408.5 | 409.2 | 2.21 |
| 16-7 | 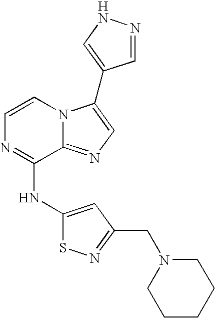 | 381.2 | 382.2 | 2.55 |
| 16-8 | 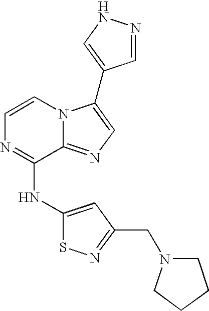 | 366.14 | 367.14 | 2.43 |

1) Walsh R. J. A.; Wooldridge, K. R. H. J. Chem. Soc. Perkin Trans. 1972, 1247.

### Example 17

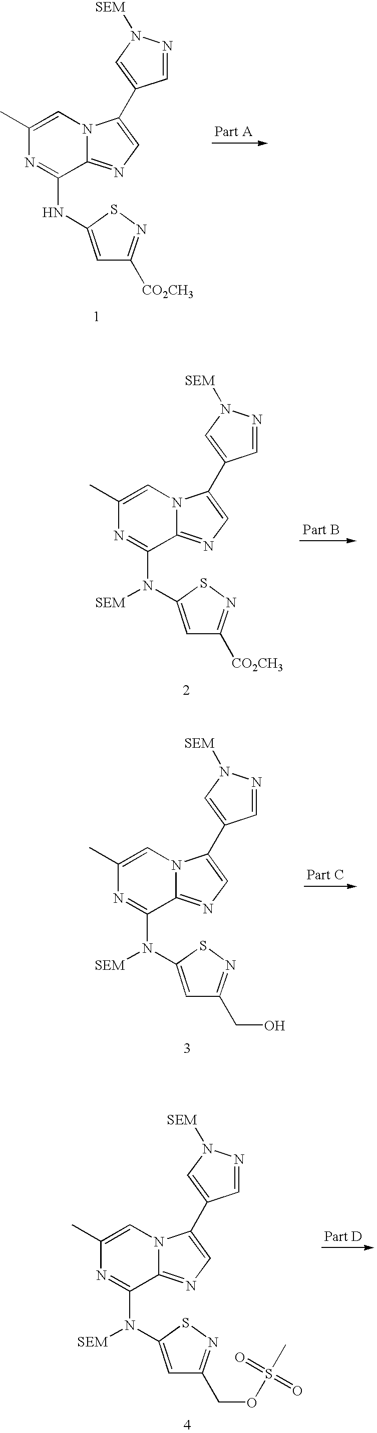

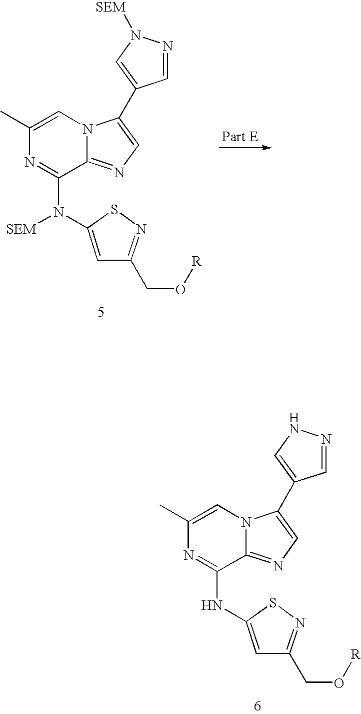

Part A: A solution of ester (2.38 g, 4.91 mmol, 1 equivalent) in DMF (40 mL) was treated with NaH (60% dispersion in oil, 1.5 equivalents) for 20 min at rt, at which time, the reaction mixture was cooled to −10 C and 2-(trimethylsilyl)ethoxymethyl chloride (0.87 mL, 1 equivalent) added to the reaction mixture. The resulting solution was allowed to slowly warm to rt and continued to stir at rt for a further 1 h. LC-MS analysis indicated the reaction was complete. The reaction was quenched with methanol (15 mL), diluted with ethyl acetate (300 mL) and washed with sat. sodium bicarbonate, water, brine, dried (sodium sulfate) and concentrated. Purification by column chromatography (SiO₂ 40% ethyl acetate/hexanes) afforded the desired product as a yellow solid 1.2 g (40%). HPLC-MS t_{R}=2.79 Min (UV₂₅₄ₘₙ). Mass calculated for formula C₂₇H₄₁N₇O₄SSi₂ 615.25, observed LC/MS m/z 616.2 (M+H).

Part B: To a solution of compound from Part A (1.2 g, 1.90 mmol, 1 equivalent) in THF (100 mL) was added superhydride solution (4 equivalents) at rt. The resulting solution was stirred at rt for 30 minutes at which time LC-MS analysis indicated the reaction was complete. The reaction was quenched with sat. aq. ammonium chloride and then extracted with dichloromethane (×2). The combined organic layers were dried (sodium sulfate) and concentrated. Purification by column chromatography (SiO₂ 60% ethyl acetate/hexanes) afforded alcohol as a clear oil (47%). HPLC-MS t_{R}=2.49 Min (UV₂₅₄ₙₘ). Mass calculated for formula C₂₆H₄₁N₇O₃SSi₂ 587.25, observed LC/MS m/z 588.3 (M+H).

Part C: A solution of alcohol from Part B (0.52 g, 0.88 mmol, 1 equivalent) in DCM (15 mL) was treated with triethylamine (1.5 equivalents) for 15 min at 0 C (ice-bath), at which time, methanesulfonyl chloride (1.2 equivalents) was added to the reaction at 0 C. The resulting solution was allowed to slowly warm to rt and continued to stir at rt for a further 3 h. LC-MS analysis indicated the reaction was complete. The reaction mixture was diluted with ethyl acetate (100 mL) and washed with water, brine, dried (anh. sodium sulfate) and concentrated to afford mesylate as a red/brown oil 0.59 g (100%) which was used without further purification. HPLC-MS t_{R}=2.66 min (UV₂₅₄ₙₘ). Mass calculated for formula C27H43N7O5S2Si2 665.23, observed LC/MS m/z 666.1 (M+H).

Part D: A solution of the respective alcohol (3 equivalents) in THF (1.5 mL) was treated with NaH (60% dispersion in oil, 2 equivalents) for 15 min at rt, at which time, mesylate from Part C (40 mg, 0.06 mmol, 1 equivalent) was added to the reaction mixture. After stirring at rt for 1 h, LC-MS analysis indicated the reaction was complete. The reaction was quenched with sat. aq. ammonium chloride and then extracted with ethyl acetate (twice). The combined organic layer was dried (sodium sulfate) and concentrated to afford crude ether, which was used without further purification.

Part E: A solution of compound from Part D in 1,4-dioxane (1 mL) was treated with 4N HCl in 1,4-dioxane solution (1 mL) at 60 C for 10 min at which time HPLC-MS indicated that the reaction was complete. The solvent was removed and the residue was purified by Prep-LC. Conversion to a hydrochloric salt afforded compounds listed in Table 3 (shown immediately below):

TABLE 3

| | | | LCMS | |
|---|---|---|---|---|
| | | | MH⁺ | HPLC |
| Example | Column 2 | MW | m/z | MS t_{R} |
| 17-1 | 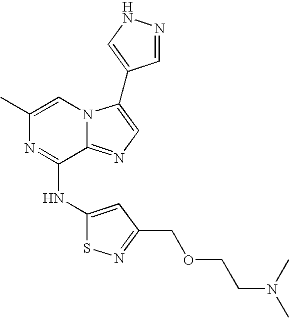 | 398.16 | 399.2 | 2.55 |
| 17-2 | 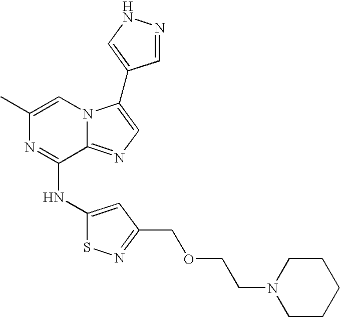 | 438.2 | 439.3 | 2.79 |
| 17-3 | 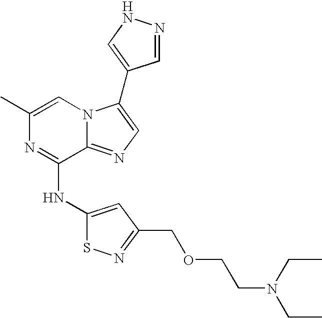 | 426.2 | 427.3 | 2.68 |
| 17-4 | 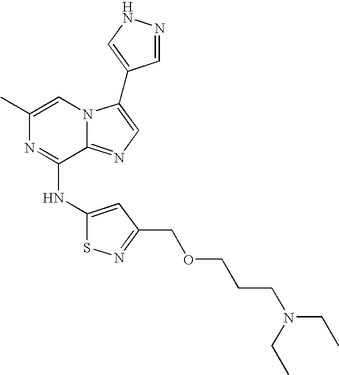 | 440.21 | 441.2 | 2.75 |
| 17-5 | 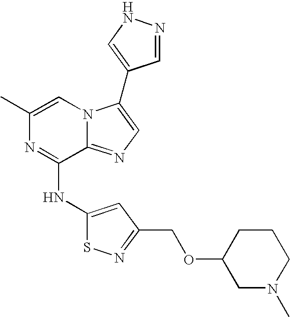 | 424.18 | 425.2 | 2.59 |
| 17-6 | 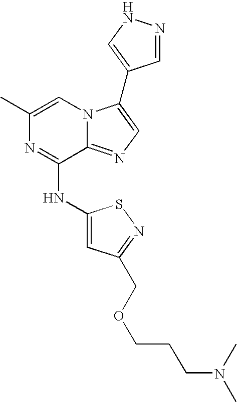 | 412.18 | 413.2 | 2.54 |
| 17-7 | 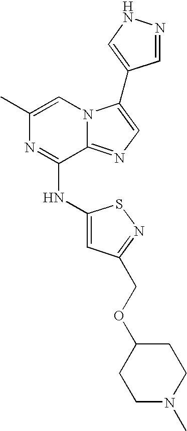 | 424.18 | 425.2 | 2.61 |

### Example 18

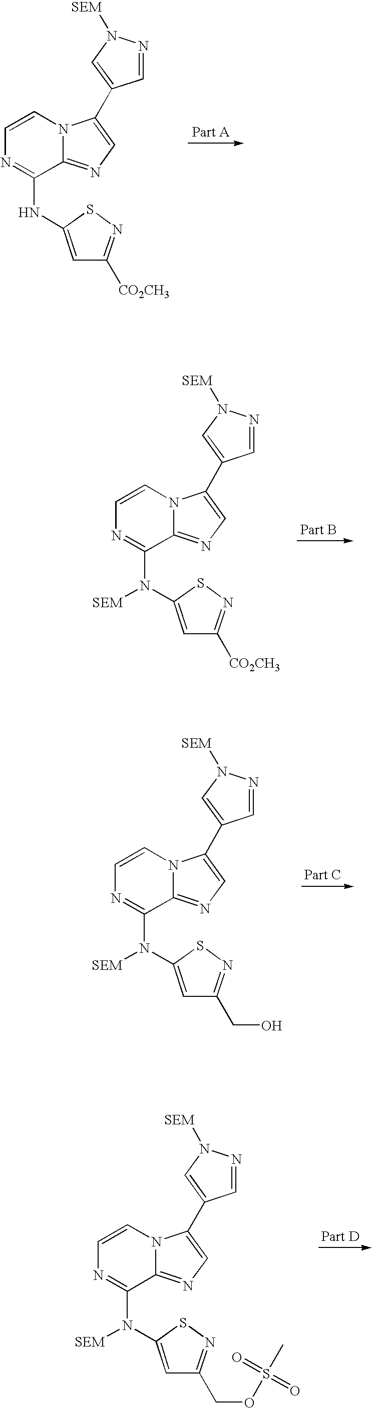

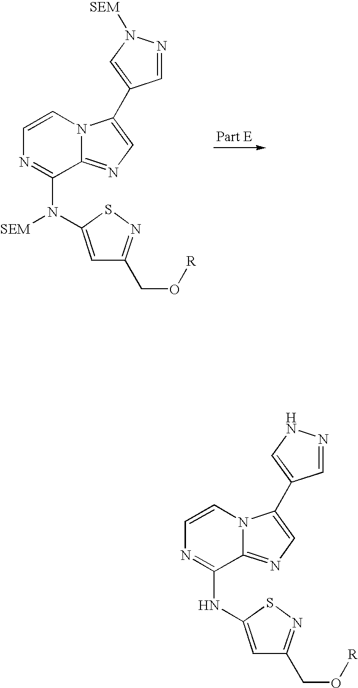

By essentially the same procedures given in Preparative Example 17, compounds given in Table 4 (shown immediately below) can be prepared.

TABLE 4

| Ex- | | | MS m/z | |
|---|---|---|---|---|
| am- | | Exact | (M + | HPLC |
| ple | Column 2 | mass | H) | MS t_{R} |
| 18-1 | 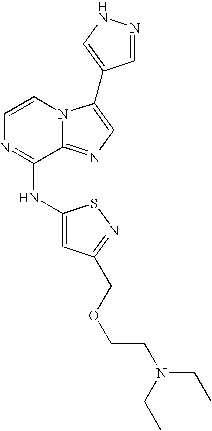 | 412.18 | 413.2 | 2.36 |
| 18-2 | 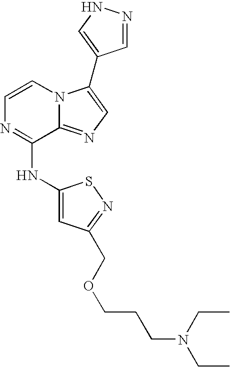 | 426.2 | 427.1 | 2.71 |
| 18-3 | 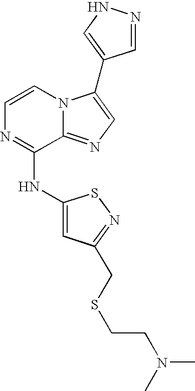 | 400.13 | 401.1 | 2.47 |
| 18-4 | 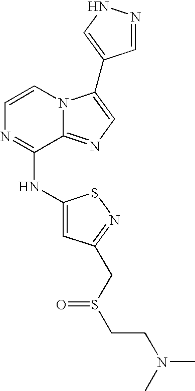 | 416.12 | 417.1 | 2.23 |

### Example 19

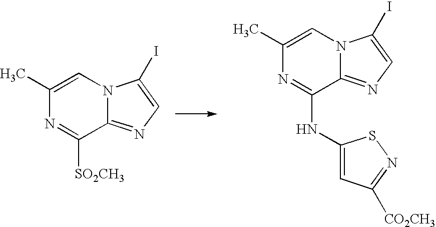

Example 19 was prepared in similar manner to Example 4. ¹H NMR (300 MHz, DMSO-d₆) δ 12.4 (bs, 1H), 7.81 (s, 1H), 7.75 (s, 1H), 7.59 (s, 1H), 3.85 (s, 3H), 2.49 (s, 3H).

### Example 20

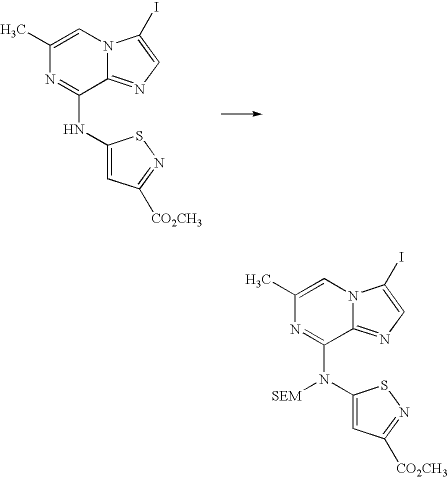

Example 20 was prepared in similar manner to Example 17, Part A. ¹H NMR (300 MHz, CDCl₃) δ 7.81 (s, 1H), 7.73 (s, 1H), 7.63 (s, 1H), 6.61 (s, 2H), 3.98 (s, 3H), 3.74 (t, J=8 Hz, 2H), 2.62 (s, 3H), 0.94 (t, J=8 Hz, 2H), −0.83 (s, 9H).

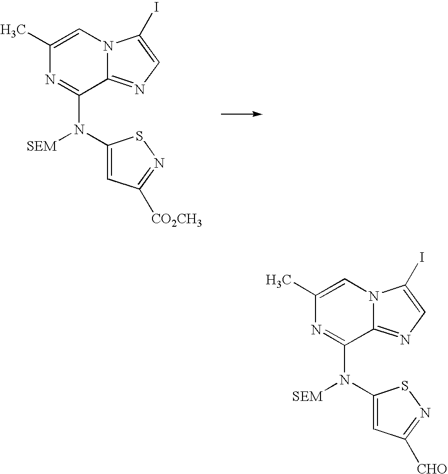

### Example 21

To a stirring solution of ester (2.40 g, 4.40 mmol) in tetrahydrofuran (96 mL) at −78° C. was added DIBAL-H (1M in dichloromethane, 11.0 mL, 11.0 mmol) dropwise. The mixture was stirred at −78° C. for 3 hours at which time thin layer chromatography (30% ethyl acetate/hexanes) indicated the reaction was complete. The mixture was quickly poured into stirring saturated aqueous sodium potassium tartrate and stirred at room temperature for 14 hours. The mixture was extracted with ethyl acetate (2×250 mL), the organic layers were combined, washed with brine (100 mL), dried over magnesium sulfate, filtered, and concentrated under reduced pressure affording compound 21 as a yellow solid 2.20 g (97%). ¹H NMR (300 MHz, CDCl₃) δ9.99 (s, 1H), 7.74 (s, 1H), 7.73 (s, 1H), 7.65 (s, 1H), 6.60 (s, 2H), 3.74 (t, J=8 Hz, 2H), 2.64 (s, 3H), 0.94 (t, J=8 Hz, 2H), −0.07 (s, 9H).

### Example 22

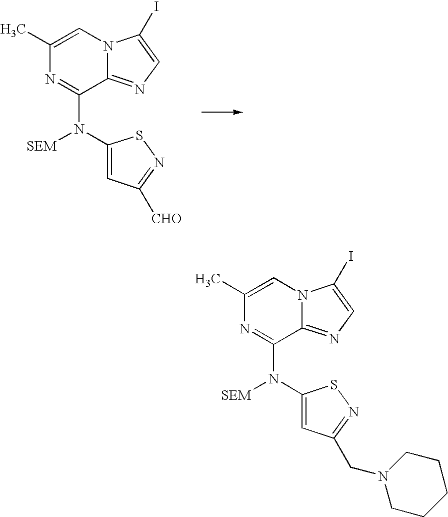

To a stirring solution of aldehyde (1.30 g, 2.52 mmol), piperidine (257 mg, 3.02 mmol), and acetic acid (150 μL, 2.52 mmol) in 1,2-dichloroethane (17 mL) at room temperature was added sodium triacetoxyborohydride (801 mg, 3.78 mmol) in one portion. The mixture was stirred at room temperature for 2 hours at which time thin layer chromatography (40% ethyl acetate/hexanes) indicated the reaction was complete. The reaction was quenched with 1N sodium hydroxide (25 mL) and stirred for 20 minutes. The mixture was extracted with chloroform (3×20 mL), the organic layers were combined, dried over sodium sulfate, filtered, and concentrated under reduced pressure. Afforded compound 22 as a yellow solid 1.35 g (92%). ¹H NMR (300 MHz, CDCl₃) δ7.70 (s, 1H), 7.59 (s, 1H), 7.23 (s, 1H), 6.58 (s, 2H), 3.72 (t, J=8 Hz, 2H), 3.62 (s, 2H), 2.58 (s, 3H), 2.47 (m, 4H), 1.58 (m, 6H), 0.93 (t, J=8 Hz, 2H), −0.086 (s, 9H).

### Example 23

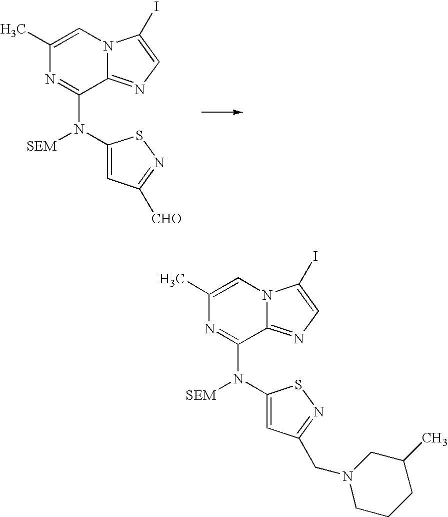

Example 23 was prepared in a similar manner to example 22 with the substitution of 3-methylpiperidine for piperidine. ¹H NMR (300 MHz, CDCl₃) δ 7.70 (s, 1H), 7.59 (s, 1H), 7.23 (s, 1H), 6.58 (s, 2H), 3.72 (t, J=8 Hz, 2H), 3.62 (s, 2H), 2.85 (m, 2H), 2.59 (s, 3H), 1.98 (m, 1H), 1.65 (m, 6H), 0.93 (t, J=8 Hz, 2H), 0.84 (d, J=6 Hz, 3H), −0.076 (s, 9H).

### Example 24

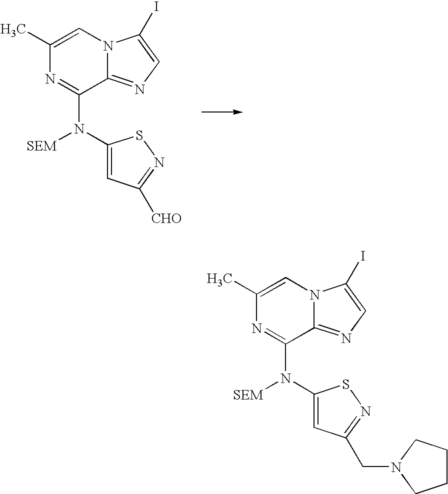

Example 24 was prepared in a similar manner to example 23 with the substitution of pyrrolidine for piperidine. ¹H NMR (300 MHz, CDCl₃) δ 7.70 (s, 1H), 7.59 (s, 1H), 7.23 (s, 1H), 6.58 (s, 2H), 3.77 (s, 2H), 3.72 (t, J=8 Hz, 2H), 2.61 (m, 4H), 2.59 (s, 3H), 1.81 (m, 4H), 0.92 (t, J=8 Hz, 2H), −0.90 (s, 9H).

### Example 25

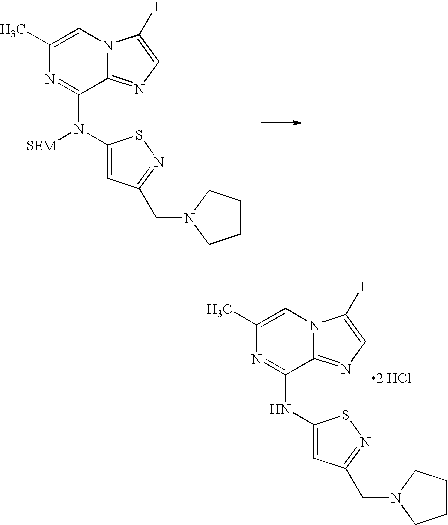

A solution of iodide from Example 24 (20 mg, 0.035 mmol) in 1,4-dioxane (1 mL) was treated with 4N HCl in 1,4-dioxane (1 mL). The mixture was sonicated at room temperature for 2.5 hours, at which time HPLC indicated the reaction was complete. The mixture was concentrated under reduced pressure and the resulting residue was purified by prep-HPLC and conversion to the hydrochloride salt afforded compound 25 as a white solid 15 mg (83%). ¹H NMR (300 MHz, CD₃OD) δ 7.88 (s, 1H), 7.79 (s, 1H), 7.17 (s, 1H), 4.51 (s, 2H), 3.71 (m, 2H), 3.22 (m, 2H), 2.57 (s; 3H), 2.07 (m, 4H). HPLC t_{R}=4.83 min (UV_{254 nm}). Mass calculated for formula C₁₅H₁₇IN₆S 440.03; observed MH⁺ (MS) 441.5 (m/z).

### Example 26

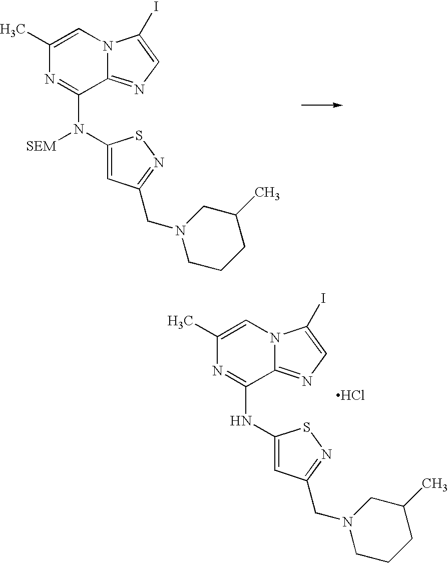

Example 26 was prepared in a similar manner to example 25. ¹H NMR (300 MHz, CD₃OD) δ 7.87 (s, 1H), 7.79 (s, 1H), 7.22 (s, 1H), 4.39 (s, 2H), 3.52 (m, 2H), 2.96 (m, 1H), 2.70 (m, 1H), 2.57 (s, 3H), 1.90 (m, 4H), 1.21 (m, 1H), 0.99 (d, J=6 Hz, 3H). HPLC t_{R}=5.06 min (UV₂₅₄ₙₘ). Mass calculated for C₁₇H₂₁IN₆S 468.3; observed MH⁺ (MS) 469.7 (m/z).

### Example 27

The compounds shown in column 2 of Table 5 (shown immediately below) were prepared as follows:

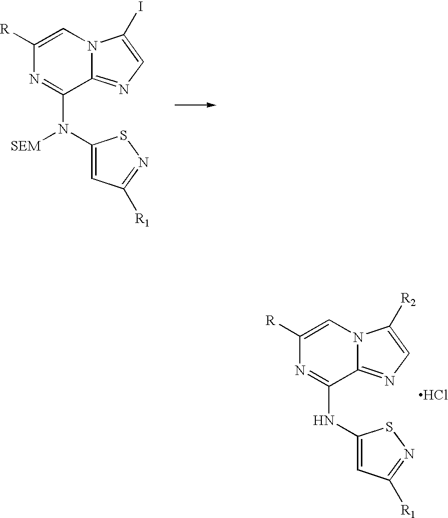

A flask containing the prepared aryl iodide scaffolds (compound from Example 22, 23, or 24, 1 equivalent), commercially available or readily prepared in 1 to 3 steps aryl/heteroaryl/alkyl boronic acid/ester/boroxine or aryl/heteroaryl/alkyl magnesium bromide or aryl/heteroaryl/alkyl zinc chloride (1.5-3 equivalents), potassium phosphate or potassium carbonate (2-3 equivalents) and Pd(PPh₃)₄ or PdCl₂dppf (0.05-0.10 equivalents) was evacuated, backfilled with nitrogen and repeated. 1,4-Dioxane or N,N-dimethylformamide or 1,2-dimethoxyethane (1-3 mL) was added and the mixture was stirred at 50-130° C. until reaction was complete as judged by thin layer chromatography (ethyl acetate/hexanes) or HPLC. The mixture was diluted with water (3-10 mL) and extracted with ethyl acetate (2-3×10-30 mL). The organic layers were combined, washed with brine (15-30 mL), dried over magnesium sulfate, filtered, concentrated, and purified by column chromatography (SiO₂, ethyl acetate/hexanes). The product obtained was dissolved in 1,4-dioxane (1 mL) and treated with 4 N HCl in 1,4-dioxane (1 mL) and sonicated at room temperature for 1-5 hours, at which time HPLC indicated the reaction was complete. The mixture was concentrated under reduced pressure and the resulting residue was purified by prep-HPLC and conversion to the hydrochloride salt afforded compounds 27-1 to 27-7.

TABLE 5

| Ex- | | | MS | |
|---|---|---|---|---|
| am- | | | MH⁺ | HPLC |
| ple | Column 2 | MW | m/z | t_{R} |
| 27-1 | 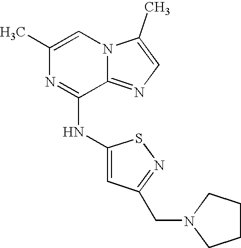 | 328.4 | 329.1 | 3.52 |
| 27-2 | 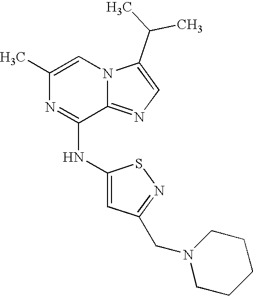 | 370.5 | 371.1 | 4.34 |
| 27-3 | 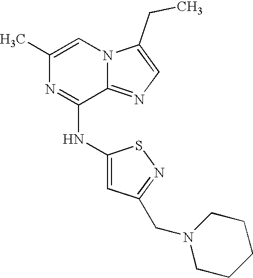 | 356.4 | 357.4 | 3.98 |
| 27-4 | 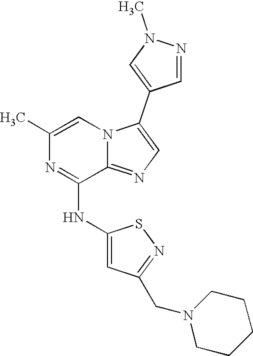 | 408.5 | 409.6 | 4.21 |
| 27-5 | 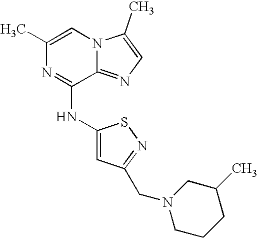 | 356.4 | 357.4 | 3.91 |
| 27-6 | 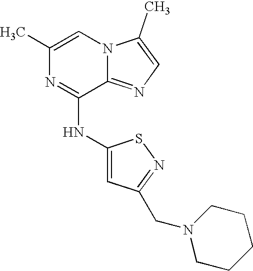 | 342.4 | 343.2 | 3.71 |
| 27-7 | 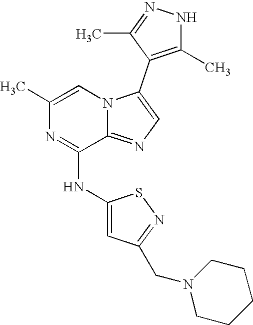 | 422.5 | 423.2 | 3.87 |

### Example 28

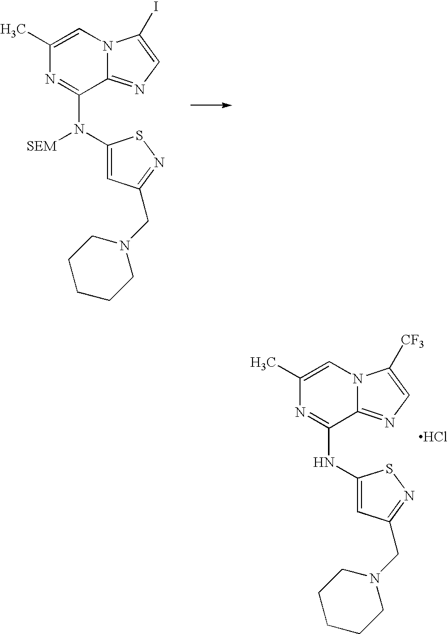

A mixture of iodide (60 mg, 0.103 mmol), trimethyl(trifluoromethyl)silane (44 mg, 0.308 mmol), copper iodide (73 mg, 0.385 mmol), potassium fluoride (15 mg, 0.257 mmol), and anhydrous DMF (1.0 mL) was degassed with nitrogen then heated at 80° C. in a sealed tube overnight. The mixture was cooled to room temperature, diluted with water (30 mL), and extracted with ethyl acetate (100 mL). The organic layer was dried over sodium sulfate, filtered, concentrated under reduced pressure, and purified by column chromatography (SiO₂, 90:10:0.25 methylene chloride/methanol/concentrated ammonium hydroxide). The resulting residue was dissolved in anhydrous 1,4-dioxane (1 mL) and 4 M HCl in dioxane (1 mL) was added. The resulting solution was sonicated at room temperature for 2 hours and concentrated under reduced pressure to dryness. Purification by preparative HPLC and conversion to the hydrochloride salt afforded the title compound as an off-white solid 3.1 mg (6%). ¹H NMR (300 MHz, CD₃OD) δ 8.07 (s, 1H), 7.85 (s, 1H), 7.24 (s, 1H), 4.40 (s, 2H), 3.61 (d, J=12.3 Hz, 2H), 3.07 (t, J=12.3 Hz, 2H), 2.55 (s, 3H), 1.75-2.03 (m, 5H), 1.56 (m, 1H). HPLC t_{R}=7.19 min. Mass calculated for formula C₁₇H₁₉F₃N₆S 396.13; observed MH⁺ 397.2 (m/z).

### Example 29

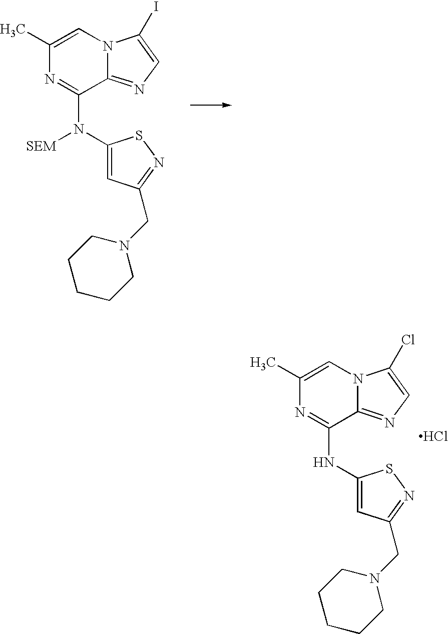

A mixture of iodide (100 mg, 0.171 mmol) in anhydrous THF (2.0 mL) was cooled to −78° C. and n-butyl lithium (2.5 M solution in hexanes, 89 μL, 0.222 mmol) was added dropwise. After stirring for 15 minutes a solution of hexachloroethane (45 mg, 0.188 mmol) in THF (1.0 mL) was added dropwise. After stirring the reaction at −78° C. for 30 minutes the solution was quenched with a saturated aqueous solution of ammonium chloride (3.0 mL) and warmed to room temperature. The reaction was concentrated under reduced pressure, extracted with ethyl acetate (50 mL) and the organic layer dried over sodium sulfate, filtered, concentrated under reduced pressure, and purified by column chromatography (SiO₂, 90:10:0.25 methylene chloride/methanol/concentrated ammonium hydroxide). The resulting residue was dissolved in anhydrous 1,4-dioxane (1 mL) and 4 M HCl in dioxane (1 mL) was added. The resulting solution was sonicated at room temperature for 2 hours and concentrated under reduced pressure to dryness. Purification by preparative HPLC and conversion to the hydrochloride salt afforded the title compound as an off-white solid 30 mg (40%). ¹H NMR (300 MHz, CD₃OD) δ 7.80 (s, 1H), 7.73 (s, 1H), 7.22 (s, 1H), 4.39 (s, 2H), 3.59 (d, J=12.3 Hz, 2H), 3.08 (t, J=12.3 Hz, 2H), 2.55 (s, 3H), 1.75-2.03 (m, 5H), 1.56 (m, 1H). HPLC t_{R}=4.79 min. Mass calculated for formula C₁₆H₂₁ClN₆S 362.11; observed MH⁺ 363.7 (m/z).

### Example 30

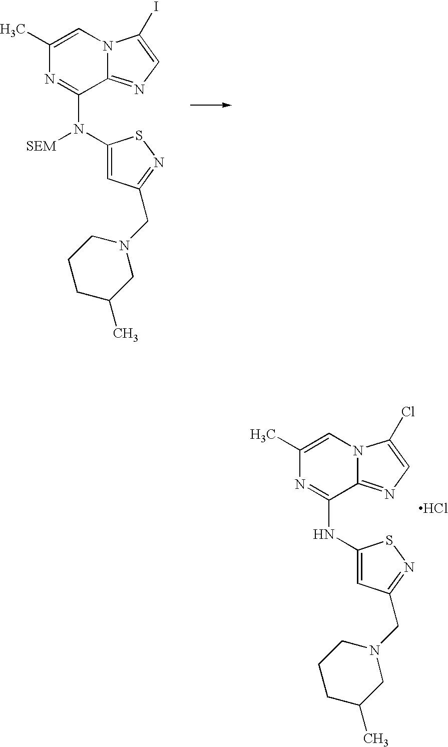

Example 30 was prepared in a similar manner as Example 29. ¹H NMR (300 MHz, CD₃OD) δ 7.77 (s, 1H), 7.68 (s, 1H), 7.20 (s, 1H), 4.39 (s, 2H), 3.47-3.67 (m, 2H), 2.97 (m, 1H), 2.71 (m, 1H), 2.55 (s, 3H), 1.77-2.01 (m, 4H), 1.20 (m, 1H), 1.00 (d, J=6.4 Hz, 3H). HPLC t_{R}=4.98 min. Mass calculated for formula C₁₇H₂₁ClN₆S 376.12; observed MH⁺ 377.6 (m/z).

### Example 31

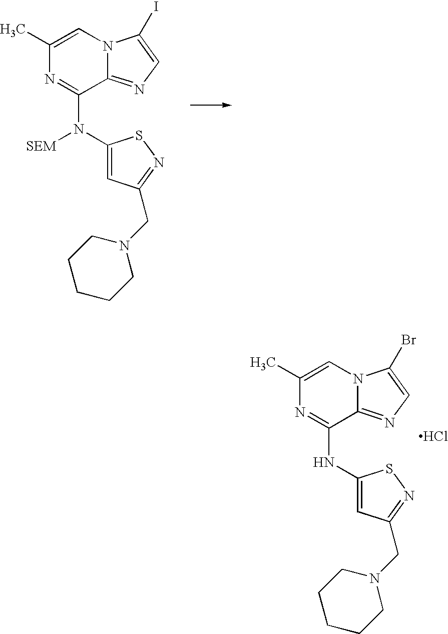

Example 31 was prepared in a similar manner to compound 29 with the substitution of tetrachlorodibromoethane for hexachloroethane. NMR (300 MHz, CD₃OD) δ 7.84 (s, 1H), 7.83 (s, 1H), 7.25 (s, 1H), 4.41 (s, 2H), 3.61 (d, J=12.3 Hz, 2H), 3.08 (t, J=12.3 Hz, 2H), 2.57 (s 3H), 1.77-2.03 (m, 5H), 1.55 (m, 1H). HPLC t_{R}=5.19 min. Mass calculated for formula C₁₆H₁₉BrN₆S 406.06; observed MH⁺ 407.4 (m/z).

### Example 32

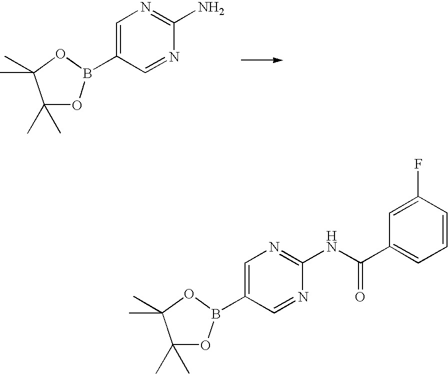

To a solution of aminopyrimidine (100 mg, 0.452 mmol) in anhydrous pyridine (2.0 mL) was added 3-fluorobenzoyl chloride (72 mg, 0.452 mmol). After stirring at room temperature overnight, the reaction was concentrated under reduced pressure, diluted with water (30 mL), and extracted with methylene chloride (100 mL). The organic layer was dried over sodium sulfate, filtered and concentrated to afford the title compound as an off-white solid 124 mg (80%). ¹H NMR (300 MHz, CDCl₃) δ 8.95 (s, 2H), 8.72 (s, 1H), 7.66-7.72 (m, 2H), 7.49 (m, 2H), 1.36 (s, 12H).

### Example 33

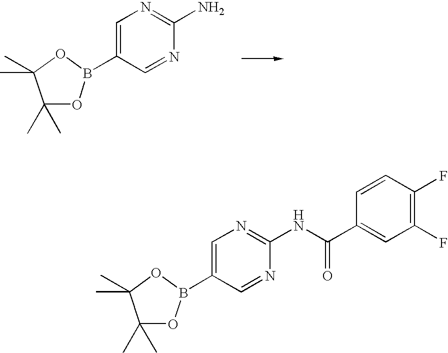

Prepared in a similar manner as compound 31 affording the title compound as an off-white solid 131 mg (80%). ¹H NMR (300 MHz, CDCl₃) δ 8.93 (s, 2H), 8.65 (s, 1H), 7.83 (m, 1H), 7.69 (m, 1H), 7.30 (m, 1H), 1.34 (s, 12H).

### Example 34

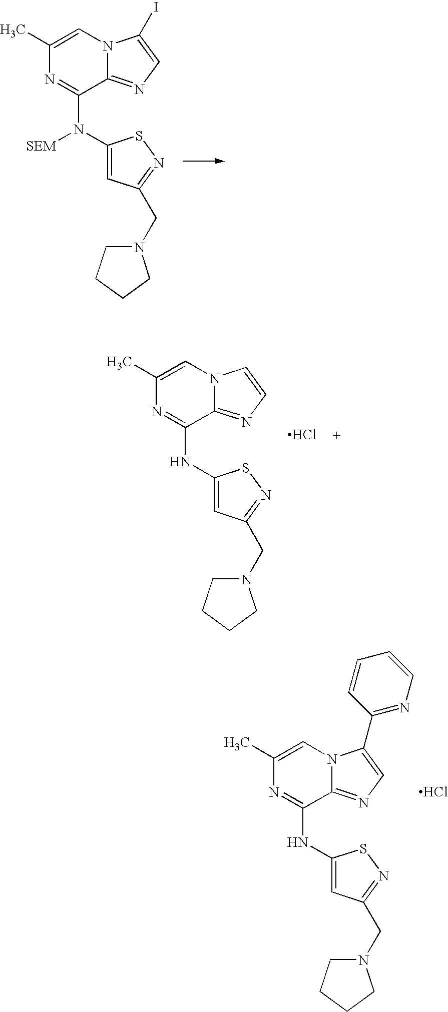

A mixture of iodide (60 mg, 0.103 mmol), tri-n-butyl(pyridyl)tin (57 mg, 0.154 mmol), dichloro[1,1′-bis(diphenylphosphino)ferrocene]palladium(II) dichloromethane adduct (8 mg, 0.0103 mmol), and potassium fluoride (18 mg, 0.309 mmol) in anhydrous 1,4-dioxane (1.0 mL) was degassed with nitrogen then heated at 85° C. in a sealed tube overnight. The mixture was cooled to room temperature, diluted with water (30 mL), and extracted with ethyl acetate (2×100 mL). The organic layer was then separated, dried over sodium sulfate, filtered, concentrated under reduced pressure to residue, and purified by column chromatography (SiO₂, 90:10:0.25 methylene chloride/methanol/concentrated ammonium hydroxide). The resulting residue was then dissolved in anhydrous 1,4-dioxane (1 mL) and 4 M HCl in dioxane (1 mL) was added. The resulting solution was sonicated at room temperature for 2 hours and then concentrated under reduced pressure to dryness. Purification by preparative HPLC and conversion to the hydrochloride salt afforded de-halogenated product 3.2 mg (10%) as an off-white solid: ¹H NMR (300 MHz, CD₃OD) δ 8.23 (s, 1H), 8.13 (s, 2H), 7.29 (s, 1H), 4.55 (s, 2H), 3.72 (br s, 2H), 3.29 (m, 2H), 2.61 (s, 3H), 2.05-2.18 (m, 4H). HPLC t_{R}=3.49 min. Mass calculated for formula C₁₅H₁₆N₆S 314.13; observed MH⁺ 315.2 (m/z).

Also afforded coupling product 5.0 mg (10%) as an off-white solid: ¹H NMR (300 MHz, CD₃OD) δ 8.95 (s, 1H), 8.86 (d, J=5.1 Hz, 1H), 8.50 (s, 1H), 8.28 (t, J=7.5 Hz, 1H), 8.18 (d, J=7.8 Hz, 1H), 7.70 (t, J=6.3 Hz, 1H), 7.26 (s, 1H), 4.54 (s, 2H), 3.74 (m, 2H), 3.29 (m, 2H), 1.99-2.30 (m, 4H). HPLC t_{R}=4.80 min. Mass calculated for formula C₂₀H₂₁N₇S 391.16; observed MH⁺ 392.5 (m/z).

### Example 35

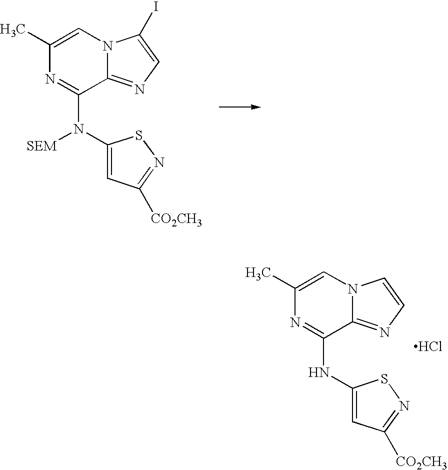

Sodium borohydride (3 mg, 0.073 mmol) was added to a room temperature suspension of iodide (15 mg, 0.037) in methanol (1 mL). The reaction was allowed to stir for 30 minutes then quenched with water (20 mL). The mixture was diluted with diethyl ether (20 mL) and the phases were allowed to separate. The organic layer was dried (sodium sulfate), filtered and concentrated to afford a protected de-halogenated intermediate. The reduced product (7 mg, 0.017 mmol) was subjected to the acidic conditions outlined previously to afford the title compound as a yellow solid 2 mg (17%). ¹H NMR (300 MHz, CD₃OD) δ 7.94 (s, 1H), 7.90 (s, 1H), 7.74 (s, 1H), 7.55 (s, 1H), 3.94 (s, 3H), 2.50 (s, 3H). HPLC t_{R}=4.67 min (UV₂₅₄ₙₘ). Mass calculated for formula C₁₂H₁₁N₅O₂S 289.06; observed MH⁻ (ESI MS) 288.0 (m/z).

### Example 36

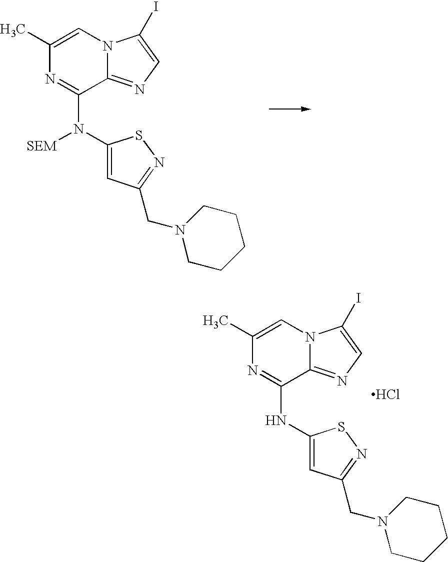

Example 36 was prepared in a similar manner to Example 31. ¹H NMR (300 MHz, CD₃OD) δ 7.72 (s, 1H), 7.69 (s, 1H), 7.15 (s, 1H), 4.38 (s, 2H), 3.69-3.52 (m, 2H), 3.17-2.96 (m, 2H), 2.54 (s, 3H), 2.06-1.70 (m, 5H), 1.65-1.44 (m, 1H). HPLC t_{R}=5.00 min (UV₂₅₄). Mass calculated for formula C₁₆H₁₉IN₆S 454.04; observed MH⁺ (ESI MS) 455.0 (m/z).

### Example 37

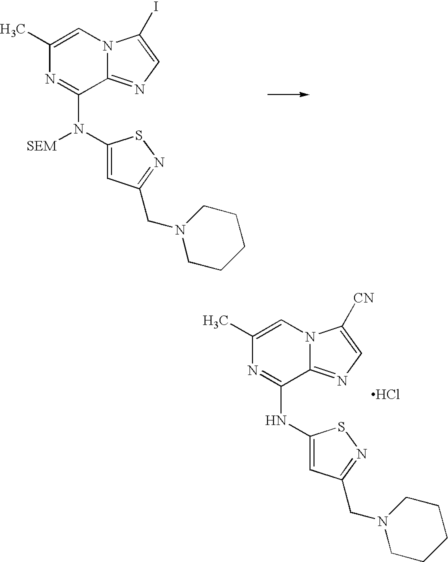

Pd₂(dba)₃ (5 mg, 0.005 mmol) was added to a room temperature solution of DPPF (6 mg, 0.103 mmol) in N,N′-dimethylformamide (1 mL) and stirred for 10 minutes. The mixture was then added to a solution of iodide (60 mg, 0.103 mmol), Zn(CN)₂ (12 mg, 0.103 mmol) in N,N′ dimethylformamide (4 mL). The reaction was heated to 150° C. in the microwave for 30 minutes, cooled to room temperature then concentrated to dryness. Purification of the resultant residue by flash chromatography (SiO₂; 12 g; 10% methanol in methylene chloride) afforded impure nitrile as a yellow solid. The impure nitrile (22 mg, 0.045 mmol) was dissolved in 2 N HCl (4 mL) without further purification. The resultant solution was sonicated at 45° C. for 2 hours. Upon completion, the reaction was concentrated to dryness. Purification of the resultant residue by prep-HPLC afforded the title compound as a white solid 8 mg (18%). ¹H NMR (300 MHz, CD₃OD) δ 8.22 (s, 1H), 7.93 (s, 1H), 7.22 (s, 1H), 4.40 (s, 2H), 3.73-3.52 (m, 2H), 3.20-2.97 (m, 2H), 2.55 (s, 3H), 2.06-1.71 (m, 5H), 1.66-1.42 (m, 1H). HPLC t_{R}=4.50 min (UV₂₅₄ₙₘ). Mass calculated for formula C₁₇H₁₉N₇S 353.14; observed MH⁺ (ESI MS) 354.3 (m/z).

### Example 38

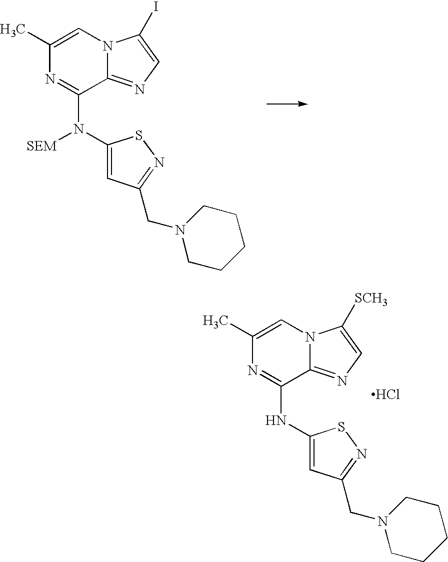

A mixture of iodide (70 mg, 0.12 mmol), Pd(dppf)Cl₂ (9 mg, 0.012 mmol), sodium tert-butoxide (35 mg, 0.36 mmol) and sodium thiomethoxide (17 mg, 0.24 mmol) was flushed with nitrogen then dissolved into 1,4-dioxane (5 mL). The solution was heated to 95° C. in the microwave for 90 minutes. The reaction was cooled to room temperature, diluted with ethyl acetate (100 mL), and filtered through celite. The organic layer was washed with water (50 mL) and brine (50 mL) then dried (sodium sulfate), filtered and concentrated to dryness. Purification of the resultant residue by prep-HPLC afforded the protected thiomethylether. The thiomethylether (35 mg, 0.07 mmol) was subjected to the reaction conditions outlined in example 110 to afford the title compound as a white solid 6 mg (11%). ¹H NMR (300 MHz, CD₃OD) δ 8.28 (s, 1H), 8.25 (s, 1H), 7.34 (s, 1H), 4.43 (s, 2H), 3.68-3.52 (m, 2H), 3.18-2.98 (m, 2H), 2.67 (s, 3H), 2.49 (s, 3H), 2.05-1.71 (m, 5H), 1.65-1.44 (m, 1H). HPLC t_{R}=4.74 min (UV₂₅₄ₙₘ). Mass calculated for formula C₁₇H₂₂N₆S₂ 374.13; observed MH⁺ (ESI MS) 375.3 (m/z).

### Example 39

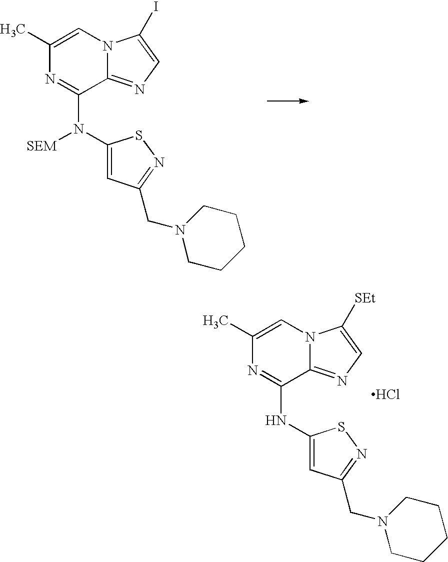

A combined mixture of iodide (70 mg, 0.12 mmol), sodium thioethoxide (20 mg, 0.24 mmol), Pd(dppf)Cl₂ (9 mg, 0.012 mmol) and sodium tert-butoxide (35 mg, 0.36 mmol) was flushed with nitrogen gas then dissolved into 1,4-dioxane (5 mL). The reaction was heated to 95° C. and stirred for 72 hours. The reaction was then cooled to room temperature, diluted with ethyl acetate (100 ml), and filtered through celite. The filtrate was washed with water (50 mL) and brine (50 mL) then dried (sodium sulfate), filtered and concentrated to dryness. Purification of the resultant residue by flash chromatography (SiO₂; 12 g; 0% to 10% methanol in methylene chloride) afforded a thioethylether intermediate as a yellow solid. The thioethylether (10 mg, 0.019 mmol) was subjected to the reaction conditions outlined in example 110 to afford the title compound as a white solid 2 mg (4%). ¹H NMR (300 MHz, CD₃OD) δ 8.32 (s, 1H), 8.28 (s, 1H), 7.35 (s, 1H), 4.43 (s, 2H), 3.68-3.55 (m, 2H), 3.17-3.01 (m, 2H), 2.90 (q, J=7.3 Hz, 2H), 2.67 (s, 3H), 2.07-1.71 (m, 5H), 1.65-1.42 (m, 1H), 1.28 (t, J=7.3 Hz, 3H). HPLC t_{R}=5.27 min (UV₂₅₄ₙₘ). Mass calculated for formula C₁₈H₂₄N₆S₂388.15; observed MH⁺ (ESI MS) 389.7 (m/z).

### Example 40

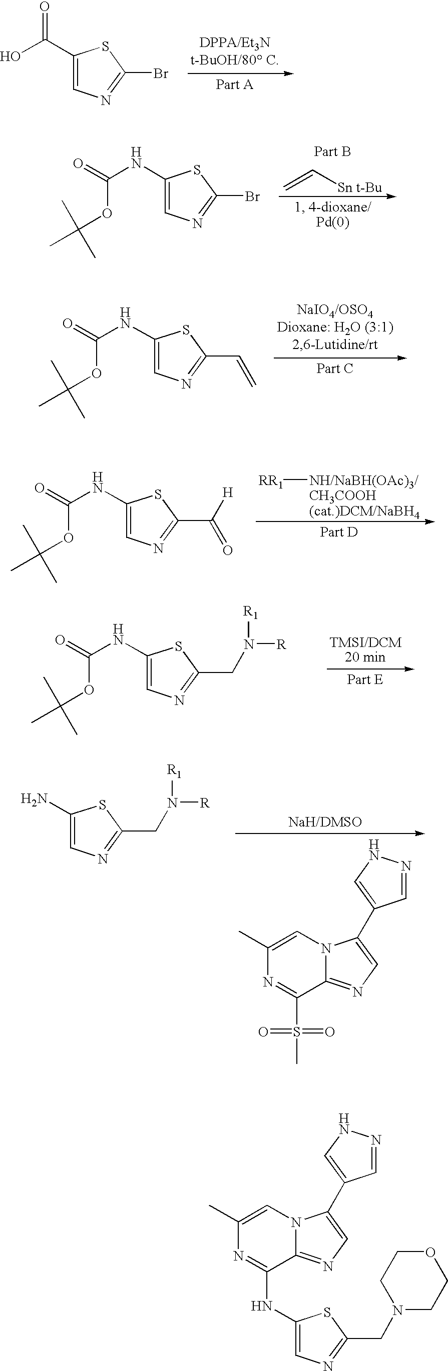

Part A: To a stirred solution of 2-Bromo-thiazole-5-carboxylic acid (2.0 g, 9.615 mmol) in t-Butanol (30 mL) and triethyl amine (1.5 mL, 10.57 mmol) was added diphenylphosphoryl azide (2.9 g, 10.57 mmol) and reaction mixture was heated to 80° C. and stirred for 12 hrs, LCMS showed the complete disappearance of the starting material. Reaction mixture was cooled to room temp., solvent removed under vacuum, water (100 mL) added and extracted with ethyl acetate (3×100 mL). Organic layer was washed with water, brine, dried over sodium sulfate and concentrated, crude material was passed through small pad of silica gel and resultant (2-Bromo-thiazol-5-yl)-carbamic acid tert-butyl ester (solid) was used as such in the next step, yield 2.5 g (90%). ¹H NMR (400 MHz, DMSO-d₆ δ 7.10 (s, 1H), 7.05 (s, 1H), 1.51 (s, 9H). Mass calculated for formula C₈H₁₁BrN₂O₂S 277.97; observed MH⁺ (LCMS) 279.0 (m/z).

Part B: To a stirred solution of (2-Bromo-thiazol-5-yl)-carbamic acid tert-butyl ester (2.5 g, 8.9928 mmol) in 1,4-dioxane (20.0 mL) were added tributy(vinyl)tin (2.9 mL, 9.892 mmol), 2,6-di-tert-butyl-4-methylphenol (cat. amt) and tetrakis(triphenyl phosphine) palladium(0) (506.0 mg, 0.4496 mmol). The reaction mixture was heated to 100° C. and stirred for 12 hrs, LCMS showed the complete disappearance of the starting material. Reaction mixture was cooled to room temp, filtered and solid washed with ethyl acetate, combined filtrate (organic solvent) was removed under vacuum, crude material was purified using biotage HPLC using hexane/ethyl acetate gradient 0.0 to 100% to yield (2-Vinyl-thiazol-5-yl)-carbamic acid tert-butyl ester (solid) 1.1 g (54%). ¹H NMR (400 MHz, CDCl₃ δ 7.27 (d, J=12.7 Hz, 2H), 7.19 (brs, 1H), 6.84-6.77 (m, 1H), 6.87 (d, J=17.0 Hz, 1H), 5.43 (d, J=10.5 Hz, 1H), 1.52 (s, 9H). Mass calculated for formula C₁₀H₁₄N₂O₂S 226.08; observed MH⁺ (LCMS) 227.1 (m/z).

Part C: To a stirred solution of (2-Vinyl-thiazol-5-yl)-carbamic acid tert-butyl ester (0.76 g, 2.857 mmol) in 1,4-dioxane:water (30:9 mL), were added sodium periodate (2.5 g, 11.43 mmol) osmium tetroxide (2.5% solution in 2-propanol) (0.5 mL) and 2,6-lutidine (0.663 mL, 5.714 mmol) and reaction mixture was stirred for 4 hrs, LCMS showed the almost disappearance of the starting material. Reaction mixture was diluted with water (100 mL) and extracted with ethyl acetate, organic layer was washed with water, brine, dried over sodium sulfate and concentrated under high vacuum to yield aldehyde 710 mg (92%). Crude product was used as such in the next reaction. ¹H NMR (400 MHz, DMSO-d₆ δ 11.45 (s, 1H), 9.76 (s, 1H), 7.58 (s, 1H), 1.40 (s, 9H). Mass calculated for formula C₉H₁₂N₂O₃S 228.06; observed MH⁺ (LCMS) 229.1 (m/z).

Part D: To a stirred solution of (2-Formyl-thiazol-5-yl)-carbamic acid tert-butyl ester (0.76 g, 2.857 mmol) in 1,2-dichloroethane (10 mL) were added Morpholine (250 mg, 1.1135 mmol) triacetoxysodium borohydride (472 mg, 2.227 mmol) and Cat amount acetic acid (three drops) and stirred for two hrs at room temp. To the reaction mixture was added sodium borohydride (126 mg, 3.3405 mmol) and stirred for one hrs. LCMS showed the disappearance of the starting material. Reaction mixture was diluted with water (100 mL) and extracted with dichloromethane, organic layer was washed with water, brine, dried over sodium sulfate and concentrated under high vacuum to yield (2-Morpholin-4-ylmethyl-thiazol-5-yl)-carbamic acid tert-butyl ester 298 mg (91%). Crude product was used as such in the next reaction. Mass calculated for formula C14H25N3O3S 315.43; observed MH⁺ (LCMS) 300.3 (m/z).

Part E: To a stirred solution of (2-Morpholin-4-ylmethyl-thiazol-5-yl)-carbamic acid tert-butyl ester (80.0 mg, 0.268 mmol) in dichloromethane (5 mL) was added iodotirmethylsilane (44 μL, 0.321 mmol) and stirred for 10 min. LCMS showed the disappearance of the starting material. Reaction mixture was diluted with water (10 mL), 1N aqueous NaOH solution (5 mL) and extracted with 10% 2-propanol in DCM (3×25 mL) and organic layer dried over sodium sulfate and concentrated under high vacuum to yield 2-Morpholin-4-ylmethyl-thiazol-5-ylamine 30.0 mg (56%). Crude product was used as such in the next reaction. Mass calculated for formula C8H13N30S 199.27; observed MH⁺ (LCMS) 200.1 (m/z).

Part F: To a stirred solution of 2-Morpholin-4-ylmethyl-thiazol-5-ylamine (30.0 mg, 0.151 mmol) in DMSO (2.5 mL) was added 8-Methanesulfonyl-6-methyl-3-(1H-pyrazol-4-yl)-imidazo[1,2-a]pyrazine (25.0 mg, 0.09045 mmol) followed by NaH 60% in mineral oil (48 mg, 1.206 mmol) and stirred for 30 min. LCMS showed the disappearance of the starting material. Reaction mixture was quenched 1:1 mixture of acetonitrile and saturated ammonium chloride (10 mL), and extracted with 10% 2-propanol in DCM (3×25 mL) and organic layer was concentrated under high vacuum to yield crude [6-Methyl-3-(1H-pyrazol-4-yl)-imidazo[1,2-a]pyrazin-8-yl]-(2-morpholin-4-ylmethyl-thiazol-5-yl)-amine which was subsequently purified by Agilent reverse phase HPLC using formic acid method to yield 10 mg (28%). HPLC-MS (10 min method) t_{R}=2.06 min (UV₂₅₄ₙₘ). Mass calculated for formula C₁₈H₂₀N₈OS 396.47; observed MH⁺ (LCMS) 397.5 (m/z).

The compounds shown in Table 6 (shown immediately below) were prepared using procedures described in Example 40.

TABLE 6

| | | | LCMS | |
|---|---|---|---|---|
| | | | MH⁺ | HPLC |
| Example | Column 2 | MW | m/z | MS t_{R} |
| 40-1 | 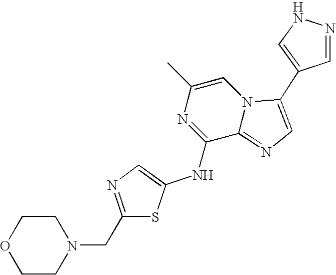 | 396.47 | 397.5 | 2.06 |
| 40-2 | 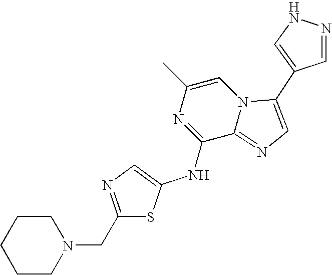 | 394.50 | 395.5 | 2.38 |
| 40-3 | 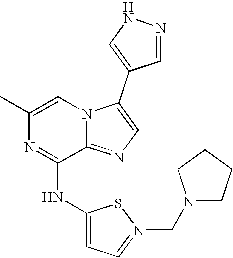 | 380.47 | 381.5 | 2.25 |
| 40-4 | 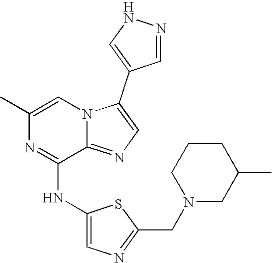 | 408.52 | 409.2 | 2.56 |
| 40-5 | 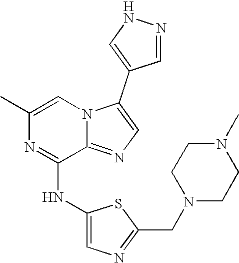 | 409.51 | 410.3 | 2.14 |
| 40-6 | 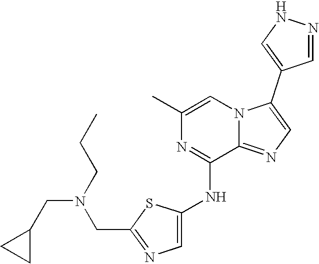 | 422.55 | 423.5 | 2.74 |
| 40-7 | 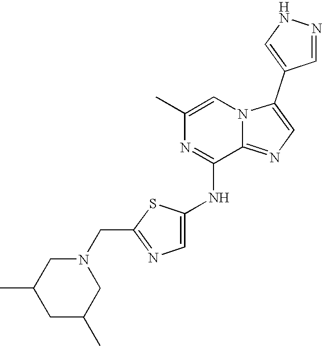 | 422.55 | 423.3 | 2.75 |
| 40-8 | 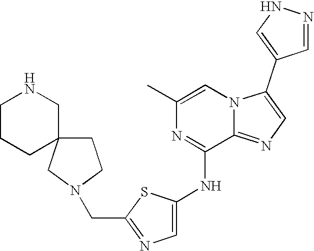 | 449.58 | 450.3 | 1.85 |
| 40-9 | 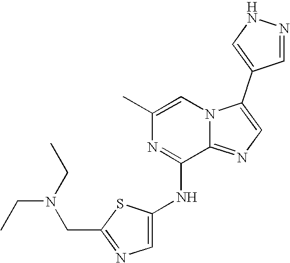 | 382.49 | 383.2 | 2.28 |
| 40-10 | 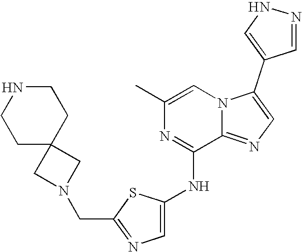 | 435.55 | 436.44 | 1.78 |
| 40-11 | 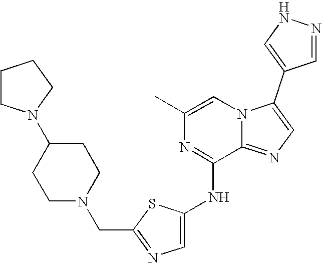 | 463.60 | 464.27 | 1.90 |
| 40-12 | 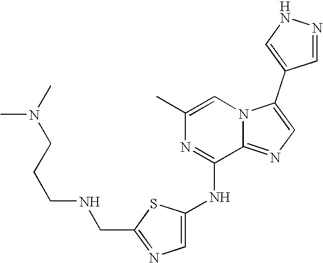 | 411.53 | 412.25 | 1.76 |
| 40-13 | 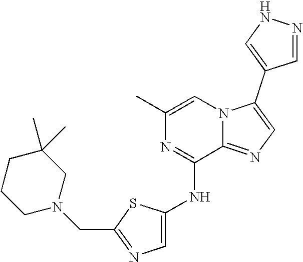 | 422.55 | 423.28 | 2.74 |

### Example 41

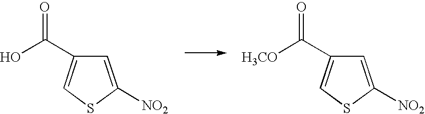

To a solution of 5-nitrothiophene-3-carboxylic acid (5.00 g, 28.88 mmol) in dimethylformamide (40 mL) was added potassium carbonate (11.98 g, 86.71 mmol) and iodomethane (2.70 mL, 43.37 mmol). The reaction mixture was stirred at RT for 16 hr. After the starting material had been consumed, the reaction was diluted with 50% ethyl acetate/hexanes (350 mL) and extracted with H₂O (350 mL). The organic layer was washed with brine (150 mL) and concentrated. Hexanes (50 mL) was added to the solid and concentrated again to yield 5.456 g (99%) of product. ¹H NMR (400 MHz) CDCl₃ δ 8.30 (s, 1H), 8.25 (s, 1H), 3.93 (s, 3H). Mass calculated for formula C₆H₅NO₄S 187.17; observed M4H⁺ (MS) 191.15 (m/z).

### Example 42

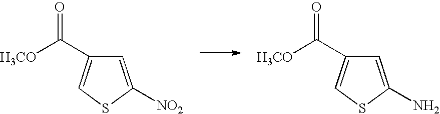

To a solution of the nitro-ester (1.006 g, 5.375 mmol) in TFA (15 mL), Fe powder (1.5135 g, 27.10 mmol) was slowly added to the round bottom flask. The reaction was heated to 60° C. for 45 min at which time TLC (1:1, ethyl acetate to hexanes) showed consumption of starting material. The reaction was diluted with ethyl acetate and the Fe was filtered off. The filtrate was neutralized with aqueous Na₂CO₃ and allowed to stir for 1 hr. The aqueous layer was extracted with EtOAc. The organic layer was washed with brine, dried over Na₂SO₄, and concentrated to give 0.701 g (83%) of a yellow solid. ¹H NMR (400 MHz) CD₃OD δ 7.29 (s, 1H), 6.44 (s, 1H), 3.79 (s, 3H). Mass calculated for formula C₆H₇NO₂S 157.19; observed MH⁺ (LCMS) 158.1 (m/z).

### Example 43

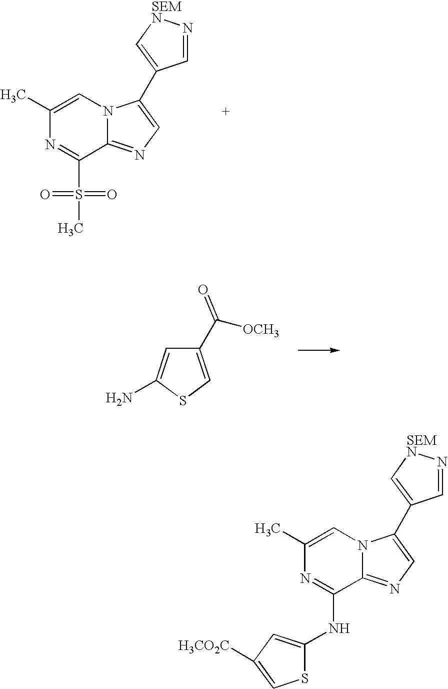

A solution of the sulfone (1.27 g, 3.11 mmol) and 2-aminothiophene-4-carboxylate methyl ester (0.701 g, 4.46 mmol) in DMF (35 mL) were treated with NaH (60% dispersion in oil, 0.402 g, 10.05 mmol) at room temperature until mass spectrometry and thin layer chromatography (50% ethyl acetate/hexanes) indicated that the reaction was complete. Saturated ammonium chloride (15 mL) and water (50 mL) were added to the reaction. The reaction was stirred for 10 minutes. The precipitated solid was collected via filtration to yield the desired product. ¹H NMR (400 MHz) CDCl₃δ 8.78 (s, 1H), 7.88 (s, 1H), 7.82 (s, 3H), 7.68 (s, 1H), 7.58 (s, 1H), 7.44 (s, 1H), 7.16 (s, 3H), 5.54 (s, 2H), 3.87 (s, 3H), 3.67 (t, 2H), 2.46 (s, 3H), 0.97 (t, 2H), 0.01 (s, 9H). Mass calculated for formula C₂₂H₂₈N₆O₃SSi 484.65; observed MH⁺ (MS) 485.1 (m/z).

### Example 44

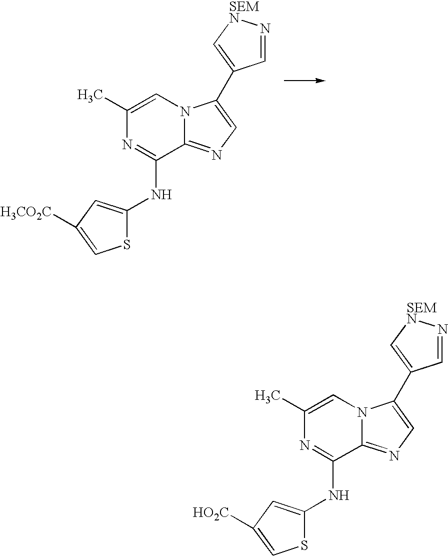

A solution of the ester prepared in Example 43 (0.565 g, 1.17 mmol) in THF (10 mL) and MeOH (3 mL) was treated with solid NaOH (9 pellets) followed by H₂O (5 mL). The reaction was stirred vigorously at room temperature for 16 hr. The THF and MeOH were removed in vacuo and the residue was extracted with EtOAc (3×20 mL). The aqueous phase was brought to a pH of 3-4 with aqueous HCl. The acidified aqueous phase was extracted with EtOAc (5×20 mL) and concentrated in vacuo to give 0.393 g (71%) of the desired carboxylic acid.

### Example 45

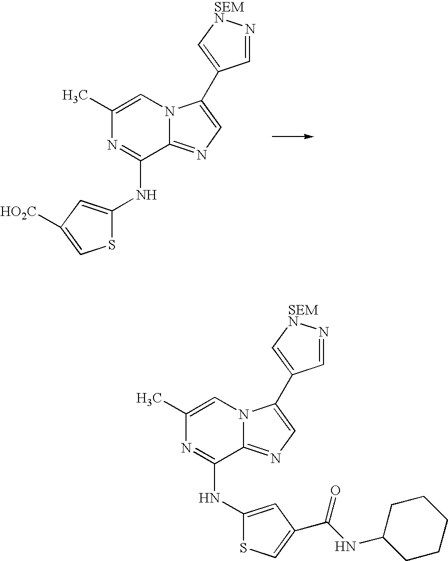

A solution of the carboxylic acid (prepared in Example 4) (0.054 g, 0.115 mmol) in DMF (3 mL) was treated with amine (0.03 mL, 0.262 mmol), NMM (0.07 mL, 0.637 mmol) and then HATU (0.141 g, 0.372 mmol). The reaction was stirred at room temperature for 16 hr. Water (15 mL) was added and the reaction was stirred for 10 minutes. The precipitated solid was collected via filtration to yield 0.038 g (60%) of the desired amide. ¹H NMR (400 MHz) CDCl₃ δ 9.13 (s, 1H), 7.86 (s, 1H), 7.80 (s, 3H), 7.54 (s, 1H), 7.41 (s, 1H), 7.31 (s, 1H), 7.08 (s, 3H), 5.83 (d, 1H), 5.53 (s, 2H), 3.93 (m, 1H), 3.67 (t, 2H), 2.44 (s, 3H), 1.70 (m, 10H), 0.96 (t, 2H), 0.00 (s, 9H). Mass calculated for formula C₂₇H₃₇N₇O₂SSi 551.78; observed MH⁺ (MS) 552.1 (m/z).

By essentially the same procedure set forth in Example 45, only substituting the amines shown in Column 2 of Table 7a (shown immediately below), the compounds in Column 3 were prepared:

TABLE 7a

| | | | | MH⁺ | LCMS |
|---|---|---|---|---|---|
| Example | Column 2 | Column 3 | MW | m/z | MS t_{R} |
| 45-1 | 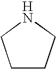 | 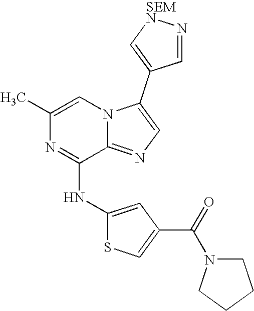 | 523.7 | 524.1 | |
| 45-2 | 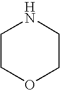 | 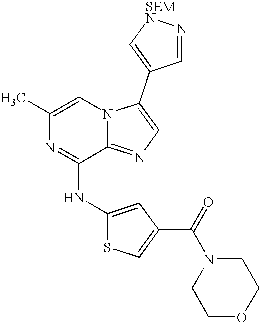 | 539.7 | 540.2 | |
| 45-3 | 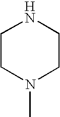 | 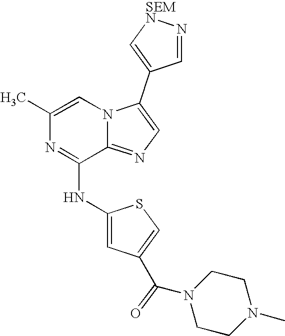 | 552.8 | 553.3 | |
| 45-4 | 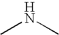 | 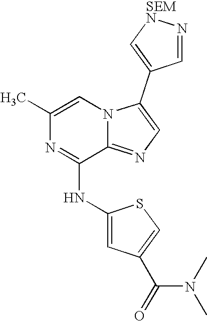 | 497.7 | 498.3 | 4.16 |
| 45-5 | 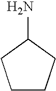 | 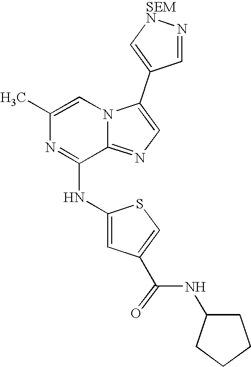 | 537.8 | 538.1 | |
| 45-6 | 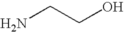 | 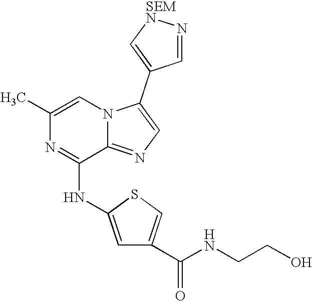 | 513.7 | 514.3 | |
| 45-7 | 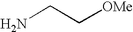 | 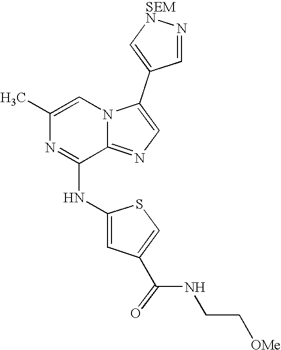 | 527.7 | 528.2 | |
| 45-8 | 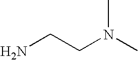 | 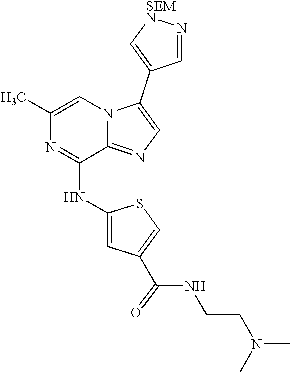 | 540.8 | 541.2 | |
| 45-9 | 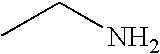 | 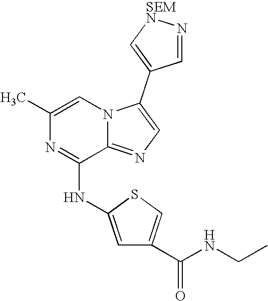 | 497.7 | 498.1 | |
| 45-10 | 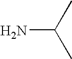 | 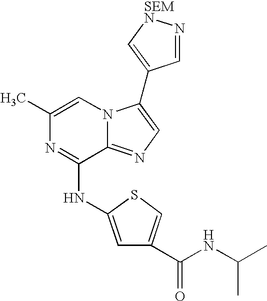 | 511.7 | 512.1 | |
| 45-11 | 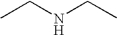 | 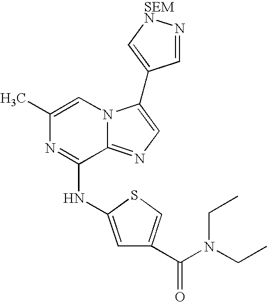 | 525.7 | 526.1 | |
| 45-12 | 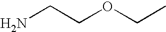 | 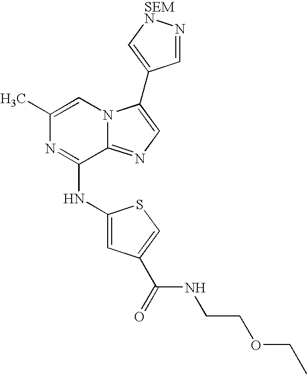 | 541.7 | 542.2 | |
| 45-13 | 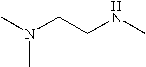 | 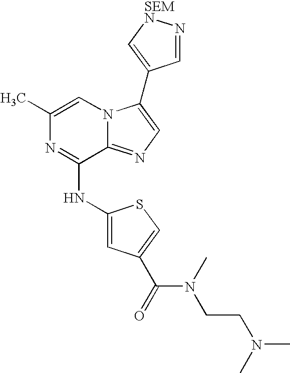 | 554.8 | 555.3 | |
| 45-14 | 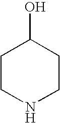 | 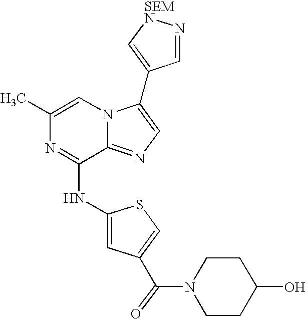 | 553.8 | 554.2 | |
| 45-15 | 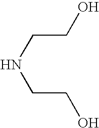 | 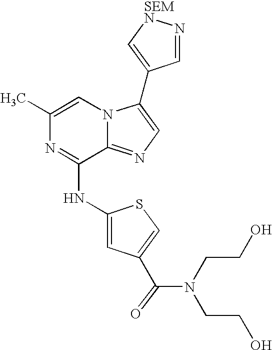 | 557.7 | 558.2 | |
| 45-16 | 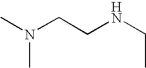 | 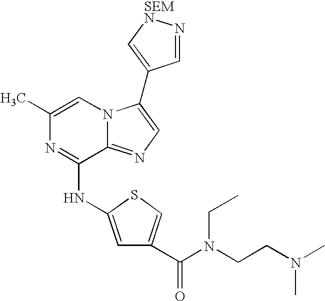 | 568.8 | 569.2 | |
| 45-17 | 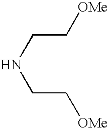 | 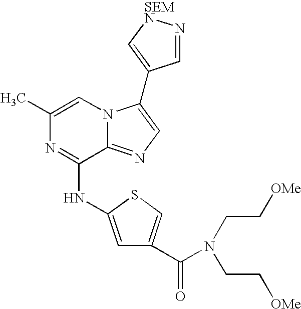 | 585.8 | 586.2 | |
| 45-18 | 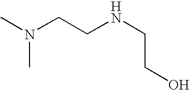 | 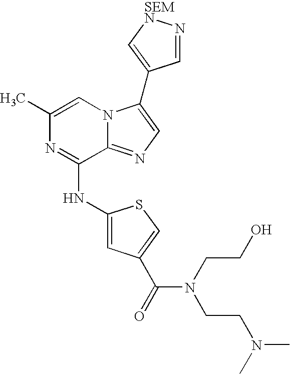 | 584.8 | 585.2 | |
| 45-19 | 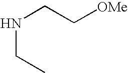 | 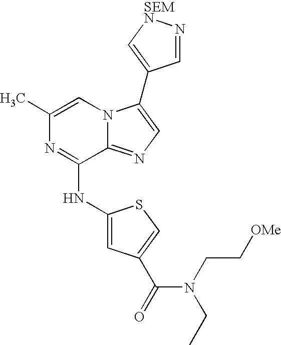 | 555.8 | 556.3 | 4.37 |

### Example 46

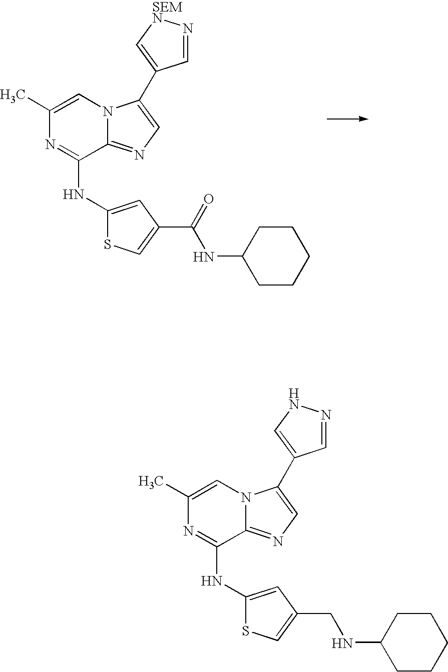

To a solution of the amide (0.038 g, 0.069 mmol) in dichloromethane (4 mL) was added lithium aluminum hydride (0.029 g, 0.775 mmol) and ethyl ether (0.8 mL). The reaction mixture was stirred at RT for 10 min then refluxed at 40° C. for 5 hr. The reaction was monitored by mass spectrometry. Upon consumption of the starting amide, the reaction was cooled to RT and quenched with H₂O (2 mL). The reaction was diluted with DCM and filtered. The filtrate was washed with H₂O (≦8 mL). The organic layer was concentrated to give 0.019 g (52%) of the amine. The above amine in THF was further treated with 4 N HCl/dioxane at 60° C. for 1 hr. Upon cooling to RT, Et₂O was added and the mixture was stirred for 10 min. The precipitated solid was collected giving 13.9 mg (96% yield) of the desired amine.

By essentially the same procedure set forth in Example 46, the following compounds shown in Column 2 of Table 7 (shown immediately below) were prepared:

TABLE 7

| | | | LCMS | |
|---|---|---|---|---|
| | | | MH⁺ | LCMS |
| Example | Column 2 | MW | m/z | MS t_{R} |
| 46-1 | 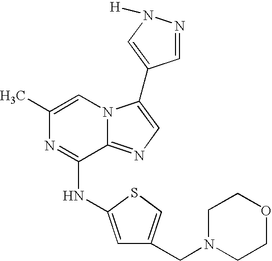 | 395.5 | 396.2 | 2.06 |
| 46-2 | 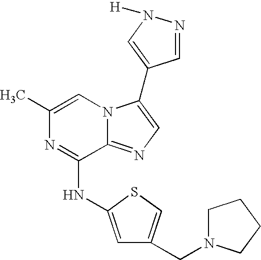 | 379.5 | 381.2 | 2.12 |
| 46-3 | 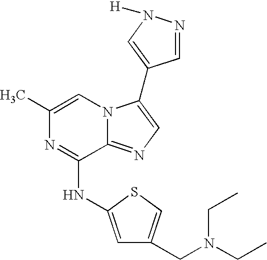 | 381.5 | 382.2 | 2.03 |
| 46-4 | 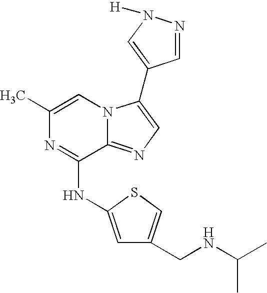 | 367.5 | 368.2 | 1.95 |
| 46-5 | 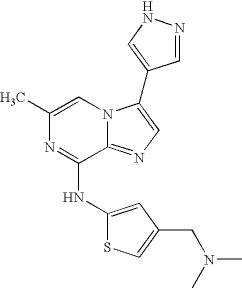 | 353.4 | 354.2 | 1.97 |
| 46-6 | 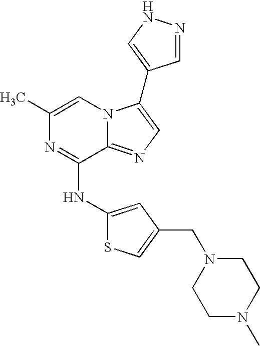 | 408.5 | 409.2 | 1.77 |
| 46-7 | 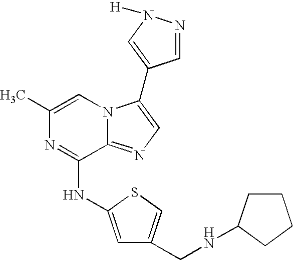 | 393.5 | 394.2 | 2.06 |
| 46-8 | 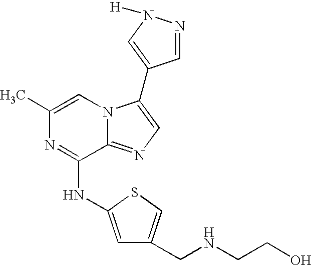 | 369.4 | 370.2 | 1.76 |
| 46-9 | 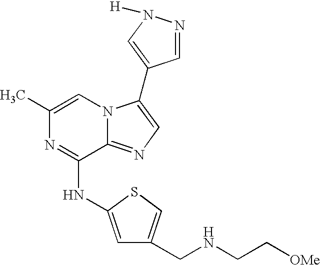 | 383.5 | 384.2 | 1.78 |
| 46-10 | 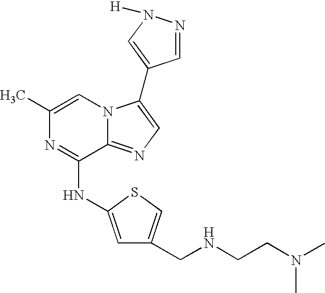 | 396.5 | 397.2 | 1.52 |
| 46-11 | 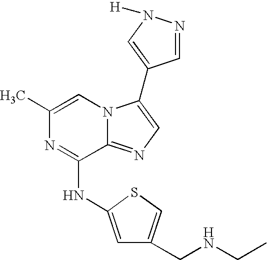 | 353.4 | 354.2 | 1.79 |
| 46-12 | 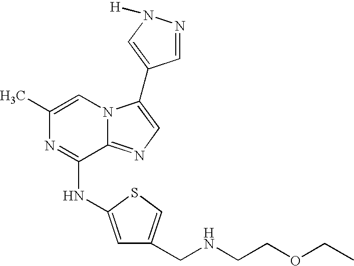 | 397.5 | 398.2 | 1.93 |
| 46-13 | 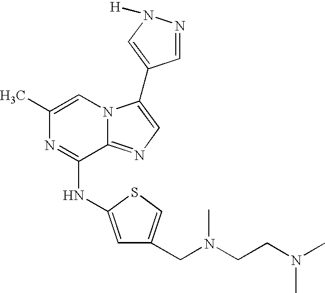 | 410.5 | 411.2 | 1.76 |
| 46-14 | 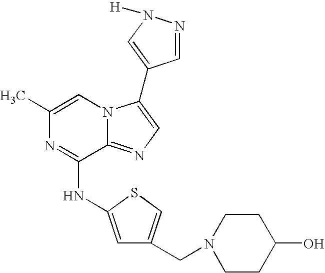 | 409.5 | 410.2 | 1.94 |
| 46-15 | 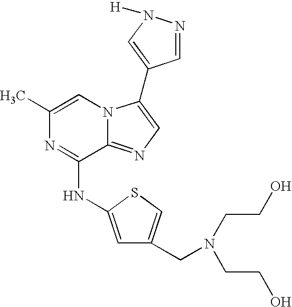 | 413.5 | 414.2 | 1.87 |
| 46-16 | 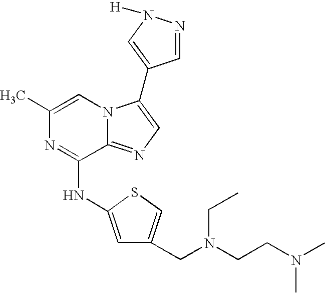 | 424.6 | 425.2 | 1.80 |
| 46-17 | 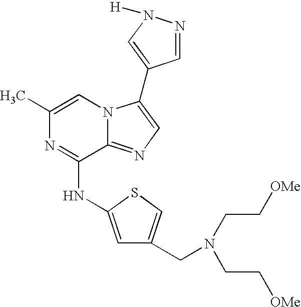 | 441.6 | 442.2 | 2.23 |
| 46-18 | 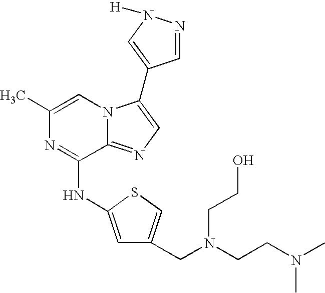 | 440.6 | 441.2 | 1.78 |

### Example 47

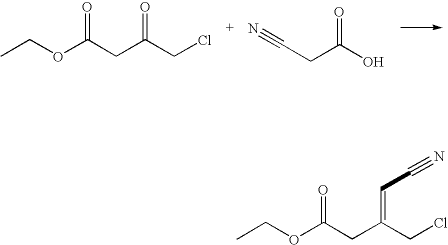

Ethyl 4-chloro-3-oxobutanoate (14.15 g, 86 mmol), cyanoacetic acid (8.00 g, 86 mmol), NH₄OAc (1.32 g, 17.2 mmol), AcOH (2.46 mL, 43 mmol), and benzene (40 mL) was stirred overnight at reflux with a Dean-Stark trap. The mixture was cooled to room temperature, diluted with EtOAc, washed with sat. NaHCO₃, brine, dried with Na₂SO₄, and concentrated to afford crude product 1 (9.29 g, 58%). HPLC-MS t_{R}=1.67 Min (UV₂₅₄ₙₘ). Mass calculated for M+ 187.0, observed LC/MS m/z 188.1 (M+H).

### Example 48

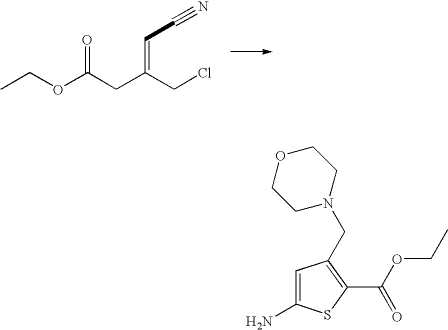

Morpholine (580 μL, 6.65 mmol) was added dropwise to a mixture of ethyl 3-(chloromethyl)-4-cyanobut-3-enoate (622 mg, 3.33 mmol) and S-flakes (116 mg, 3.63 mmol) in EtOH (5 mL). The reaction stirred at room temperature overnight. The mixture was concentrated. The mixture was diluted with EtOAc, washed with brine, dried with Na₂SO₄, and concentrated to afford crude product. Purification by Prep-LC afforded the title compound (182 mg, 20%). HPLC-MS t_{R}=0.80 Min (UV₂₅₄ₙₘ). Mass calculated for M+ 270.1, observed LC/MS m/z 271.1 (M+H).

### Example 49

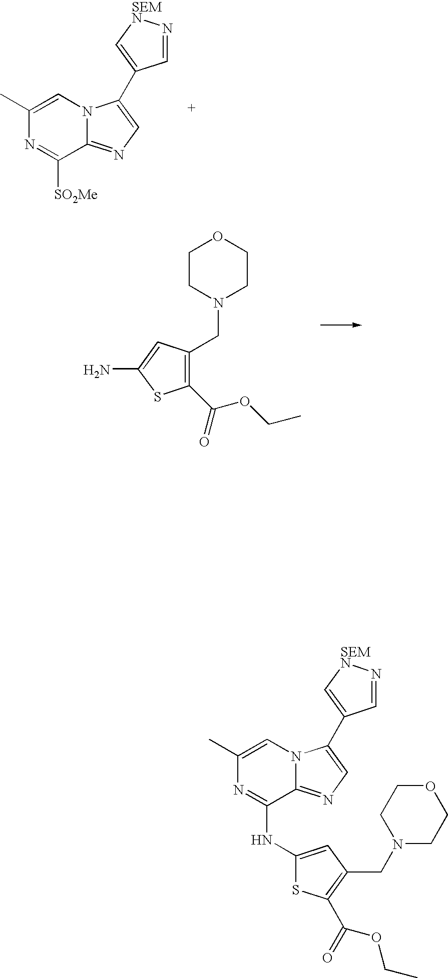

A solution of ethyl 5-amino-3-(morpholinomethyl)thiophene-2-carboxylate (61.0 mg, 0.225 mmol) and sulfone (71.0 mg, 0.173 mmol) in DMF (2 mL) was treated with NaH (60% dispersion in oil, 20.9 mg, 0.521 mmol) at room temperature. The mixture was stirred at room temperature until LCMS indicate the reaction was complete. The reaction mixture was diluted with EtOAc, washed with sat NH₄Cl, dried with Na₂SO₄, and concentrated to afford title compound. HPLC-MS t_{R}=1.79 Min (UV₂₅₄ₙₘ). Mass calculated for M+ 597.2, observed LC/MS m/z 598.3 (M+H).

### Example 50

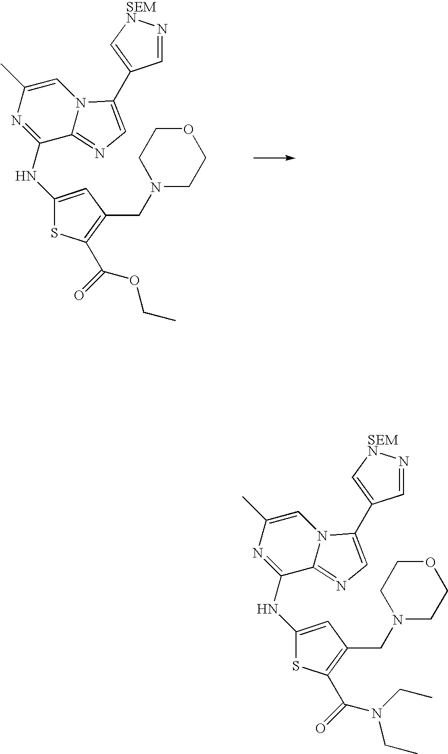

A solution of i-PrMgCl in THF (0.78 μL, 1.56 mmol) was added dropwise to a solution of crude compound from example 49 (104.2 mg, 0.173 mmol) and diethylamine (91 μL, 0.782 mmol) in THF (3 mL) at −20° C. The mixture was slowly warmed up to room temperature and stirred at this temperature until LCMS indicate the reaction was complete. The reaction mixture was cooled to 0° C. and quenched with Sat. NH₄Cl. The reaction mixture was extracted with EtOAc and the organic layer was dried with Na₂SO₄ and concentrated to afford crude product 4. HPLC-MS t_{R}=1.81 Min (UV₂₅₄ₙₘ). Mass calculated for M+ 624.3, observed LC/MS m/z 625.3 (M+H).

### Example 51

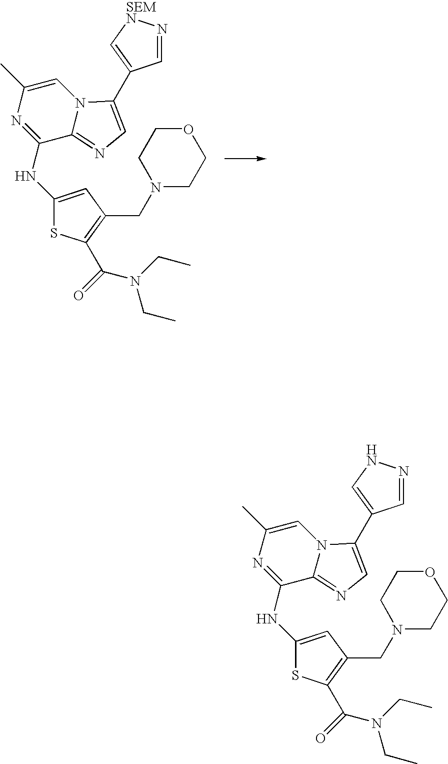

4N HCl in dioxane (1 mL) was added to crude compound 4 (17 mg, 0.027 mmol) at 0° C. The mixture was warmed to room temperature and stirred at this temperature until LCMS indicate the reaction was complete. Concentration and purification by Prep-LC and conversion to a hydrochloric salt afforded the title compound. HPLC-MS t_{R}=1.14 Min (UV₂₅₄ₙₘ). Mass calculated for M+ 494.2, observed LC/MS m/z 495.2 (M+H).

By essentially the same procedure, compounds given in Column 2 of Table 8 (shown immediately below) can be prepared.

TABLE 8

| Ex- | | | LCMS | |
|---|---|---|---|---|
| am- | | | MH⁺ | LCMS |
| ple | Column 2 | MW | m/z | MS t_{R} |
| 51-1 | 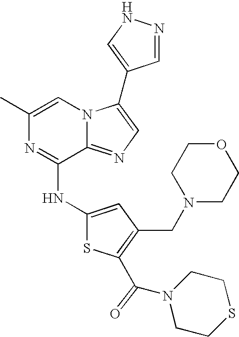 | 524.2 | 525.2 | 1.328 |
| 51-2 | 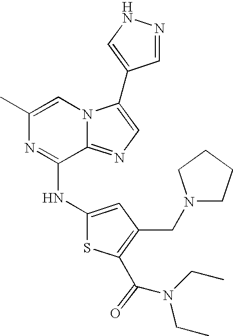 | 478.2 | 479.2 | 1.355 |
| 51-3 | 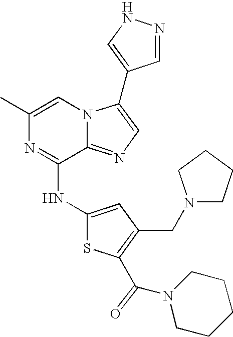 | 490.2 | 491.2 | 1.099 |

### Example 52

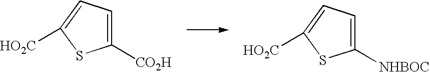

A solution of thiophene-2,5-dicarboxylic acid (2.73 g, 15.84 mmol), diphenylphosphoryl azide (3.41 mL g, 15.84 mmol) and triethylamine (4.4 mL, 31.68 mmol) in tert-butanol (80 mL) was heated at refluxing 5 h. The reaction mixture was cooled to room temperature and then concentrated to afford the crude title compound. HPLC-MS t_{R}=1.52 Min (UV₂₅₄ₙₘ). Mass calculated for M+ 243.0, observed LC/MS m/z 244.1 (M+H).

### Example 53

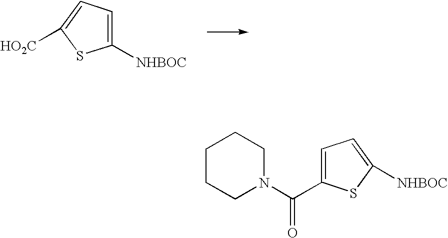

Et₃N (1261.6 μL, 9.05 mmol) was added at 0° C. to a mixture of 5-tert-Butoxycarbonylamino-thiophene-2-carboxylic acid (550 mg, 2.26 mmol), EDCI (1086 mg, 5.65 mmol), and piperidine (447 μL, 4.52 mmol) in DMF (6 ml). The reaction mixture was warmed up to room temperature and stirred at this temperature overnight. The mixture was diluted with EtOAc, washed with brine (2×), dried over Na₂SO₄ and concentrated to afford crude residue. Purification by Biotage afforded compound 2 (368 mg, 53%). HPLC-MS t_{R}=1.89 Min (UV₂₅₄ₙₘ). Mass calculated for M+ 310.1, observed LC/MS m/z 311.2 (M+H). HPLC-MS t_{R}=2.4 Min (UV₂₅₄ₙₘ).

### Example 54

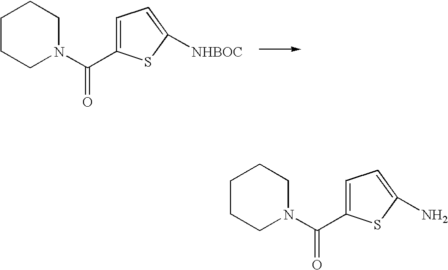

Compound from Example 53 (90 mg, 0.29 mmol) was stirred in 20% TFA/CH₂Cl₂ solution (5 mL) at room temperature for 1.5 hrs. The reaction mixture was concentrated to afford compound 3. The crude product was used without further purification. HPLC-MS t_{R}=1.16 Min (UV₂₅₄ₙₘ). Mass calculated for M+ 210.0, observed LC/MS m/z 211.1 (M+H).

### Example 55

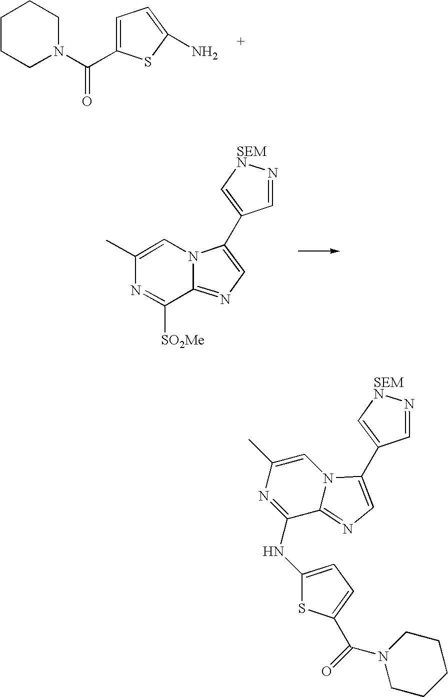

A solution of crude material from Example 54 and sulfone (98.0 mg, 0.241 mmol) in DMF (2 mL) was treated with NaH (60% dispersion in oil, 29.0 mg, 0.725 mmol) at room temperature. The mixture was stirred until LCMS indicate the reaction was complete. The reaction mixture was diluted with EtOAc, washed with sat NH₄Cl, dried with Na₂SO₄, and concentrated to afford crude product 4. Purification by Biotage afforded the title compound (82 mg, 63%). HPLC-MS t_{R}=2.30 Min (UV₂₅₄ₙₘ). Mass calculated for M+ 537.2, observed LC/MS m/z 538.2 (M+H).

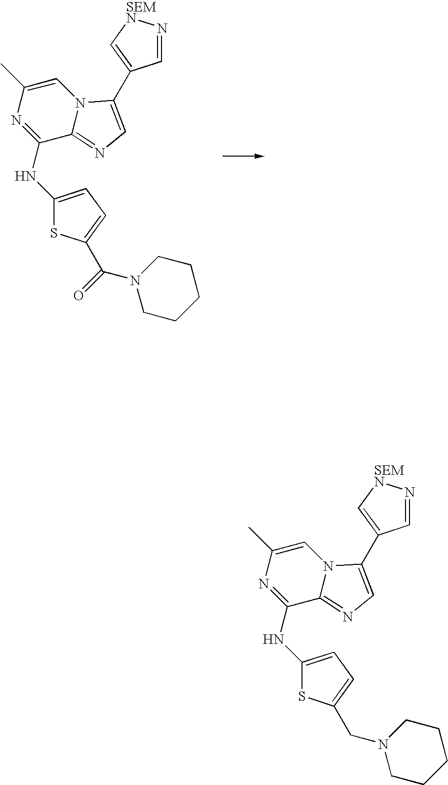

### Example 56

To a solution of the amide (47.6 mg, 0.089 mmol) in dichloromethane (5 mL) was added lithium aluminum hydride (39.9 mg, 1.0 mmol) and ethyl ether (1 mL). The reaction mixture was stirred at room temperature for 10 min then refluxed at 40° C. until LCMS indicate the reaction was complete. The reaction was cooled to room temperature and quenched with H₂O (0.5 mL). The reaction was diluted with dichloromethane, dried over Na₂SO₄ and concentrated to afford crude title compound. HPLC-MS t_{R}=1.52 Min (UV₂₅₄ₙₘ). Mass calculated for M+523.2, observed LC/MS m/z 524.2 (M+H).

### Example 57

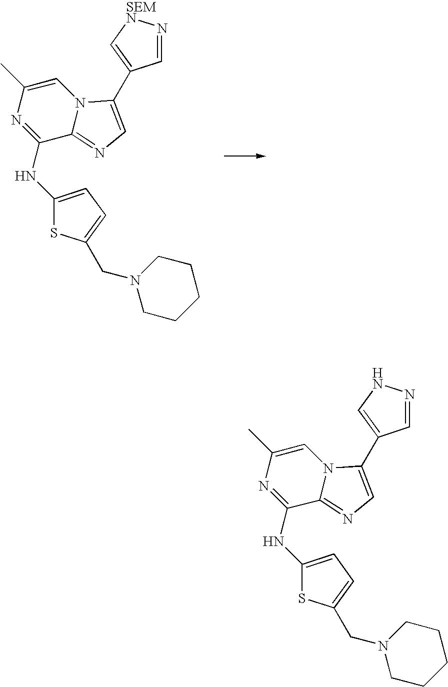

4N HCl in dioxane (2 mL) was added to crude compound from example 56 at 0° C. The mixture was warmed to room temperature and stirred at this temperature until LCMS indicate the reaction was complete. Concentration afforded crude title compound. Purification by Prep-LC and conversion to a hydrochloric salt afforded the title compound. HPLC-MS t_{R}=0.91 Min (UV₂₅₄ₙₘ). Mass calculated for M+ 393.1, observed LC/MS m/z 394.1 (M+H).

### Example 58

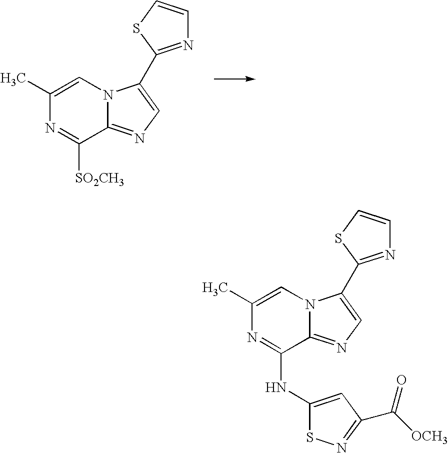

To a solution of 8-methanesulfonyl-6-methyl-3-thiazol-2-yl-imidazo[1,2-a]pyrazine (0.070 g; 0.24 mmol) and 5-amino-3-carbomethoxy-isothiazole (0.039 g; 0.25 mmol) in dimethyl formamide (DMF; 0.8 mL) was added sodium hydride (NaH; 60% in oil; 0.024 g). The reaction mixture was stirred at room temperature for 0.5 hr and then quenched with saturated aqueous solution of ammonium chloride (NH₄Cl). Diluted with more water and filtered. The filter cake was washed with water and hexanes. The filter cake was dried in vacuo to obtain the title compound as yellow solid (0.078 g; 87%). ¹H NMR (400 MHz, DMSO-d₆): 8.85 (s, 1H), 8.42 (s, 1H), 8.1 (s, 1H), 7.9 (s, 1H), 7.65 (s, 1H), 3.85 (s, 3H) 2.5 (s, 3H). HPLC-MS t_{R}=4.35 (UV₂₅₄ₙₘ). Mass Calc. for C₁₅H₁₂N₆O₂S₂ 372.04; obsd MH⁺ (LCMS) 373.2 (m/z).

### Example 59

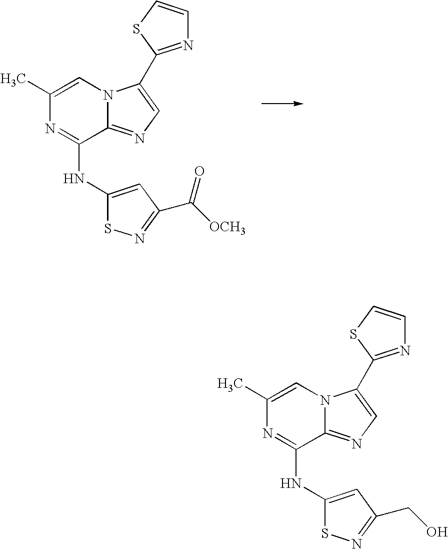

A solution of lithium triethylborohydride (Super Hydride; 1M in THF; 0.32 mL) was added dropwise to a solution of the methyl ester (0.03 g; 0.08 mmol) in dry THF (0.8 mL). After stirring at room temperature for 1.5 hr, the reaction mixture was quenched with saturated aqueous NH₄Cl solution (8 mL) and diluted with water. Small amount of the precipitated yellow solid was filtered and washed with water and ether. The solid was dried in vacuo to obtain ˜10 mg (36%) of the alcohol. ¹H NMR (400 MHz, DMSO-d₆): 8.8 (s, 1H), 8.4 (s, 1H), 8.1 (s, 1H), 7.9 (s, 1H), 7.2 (s, 1H), 5.4 (t, 1H). 4.5 (d, 2H), 2.5 (s, 3H). HPLC-MS t_{R}=2.98 (UV₂₅₄ₙₘ). Mass Calc. for C₁₄H₁₂N₆OS₂ 344.05; obsd MH⁺ (LCMS) 345.2 (m/z).

### Example 60

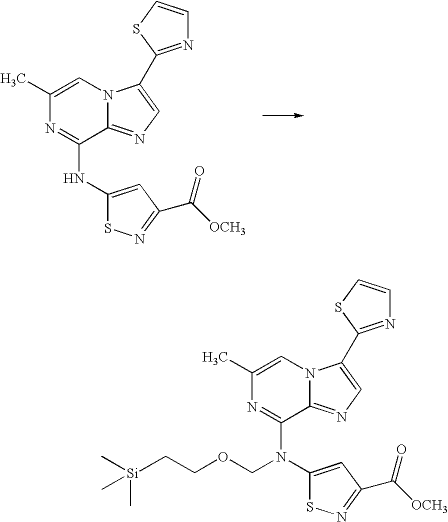

A solution of ester (0.113 g; 0.3 mmol) in DMF (1.5 mL) was treated with NaH (60% in oil; 0.03 g; 0.76 mmol) followed by 2-(Trimethylsilyl)ethoxymethyl chloride (SEM-Cl; 0.1 mL; 0.61 mmol). The reaction mixture was stirred at room temperature for 3 hr and quenched with saturated aqueous NH₄Cl and water. The precipitated yellow solid was collected by filtration, washed with water and dried. The title compound was obtained as yellow solid (0.142 g; 92%). ¹H NMR (400 MHz, CDCl₃): 9.1 (s, 1H), 8.1 (s, 1H), 7.98 (s, 1H), 7.8 (s, 1H), 7.4 (s, 1H), 6.65 (s, 2H), 4.0 (s, 3H), 3.78 (t, 2H), 2.65 (s, 3H), 1.0 (t, 2H), 0.0 (s, 9H). HPLC-MS t_{R}=5.98 (UV₂₄₅ₙₘ). Mass Calc. for C₂₁H₂₆N₆O₃S₂Si 502.13; obsd MH⁺ (LCMS) 503.3 (m/z).

### Example 61

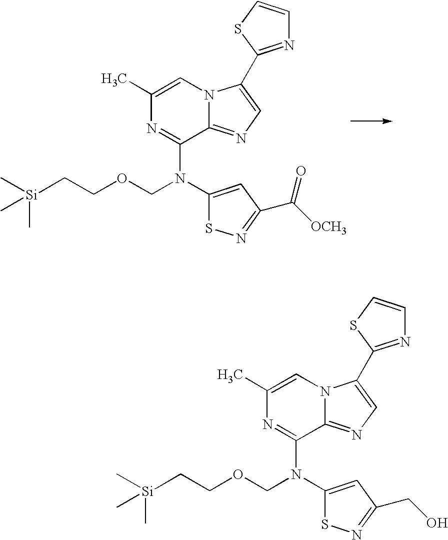

A solution of lithium triethylborohydride (Super Hydride; 1M in THF; 1 mL) was added dropwise to a solution of the methyl ester 2 in dry THF. After stirring at room temperature for 1 hr, the reaction mixture was quenched with saturated aqueous NH₄Cl solution (8 mL) and saturated aqueous solution of Rochelle salt. The organic product was extracted with dichloromethane (CH₂Cl₂), washed with water and brine. Concentration in vacuo gave ˜120 mg (100%) of the alcohol.

HPLC-MS t_{R}=4.22 (UV₂₅₄ₙₘ). Mass Calc. for C₂₀H₂₆N₆O₂S₂Si 474.13; obsd MH⁺ (LCMS) 475.3 (m/z).

### Example 62

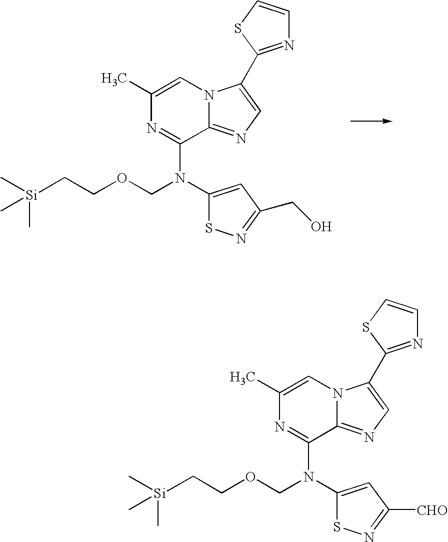

Dess-Martin periodinane (0.147 g; 0.35 mmol) was added to a solution of alcohol (0.11 g; 0.23 mmol) in dry THF and stirred at room temperature for 40-minutes. The reaction mixture was diluted with 30 mL of CH₂Cl₂, washed with saturated sodium bicarbonate (NaHCO₃) solution, water and dried. Concentration furnished a yellow solid which was re-dissolved in CH₂Cl₂ and filtered. The filtrate was concentrated to obtain 120 mg of crude title compound as a yellow solid which was used as is in the next step. ¹H NMR (400 MHz, CDCl₃): 10 (s, 1H), 9.1 (s, 1H), 8.1 (s, 1H), 7.98 (s, 1H), 7.78 (s, 1H), 7.4 (s, 1H), 6.6 (s, 2H), 3.78 (t, 2H), 2.65 (s, 3H), 1.0 (t, 2H), 0.0 (s, 9H). HPLC-MS t_{R}=6.14 (UV₂₅₄ₙₘ). Mass Calc. for C₂₀H₂₄N₆O₂S₂Si 472.12; obsd MH⁺ (LCMS) 473.3 (m/z).

### Example 63

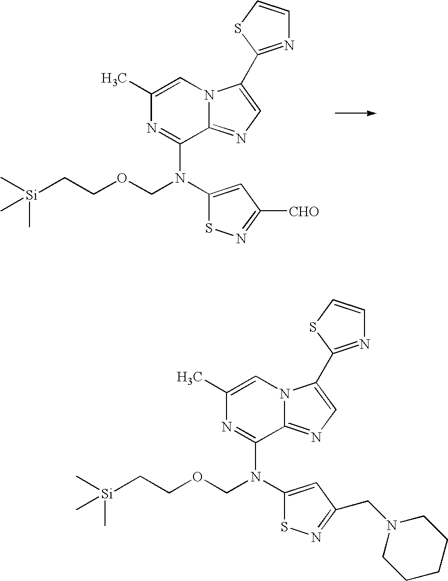

A solution of aldehyde (0.05 g; 0.1 mmol) and piperidine (0.05 mL; 0.5 mmol) in CH₂Cl₂ (1 mL) was treated with glacial acetic acid (AcOH; 1 drop) and stirred at room temperature (RT) for 3 hr. Solid sodium borohydride (NaBH₄; 0.016 g; 0.42 mmol) was added and the reaction mixture was cooled in an ice/brine bath (−5° C.) and methanol (0.2 mL) was added dropwise. After stirring at low temperature for 30 minutes, the reaction was quenched with saturated NH₄Cl and extracted into CH₂Cl₂. The organic extract was washed with saturated NH₄Cl, water and brine. Removal of solvent gave the crude product which was purified by preparative thin layer chromatography (Prep TLC) using CH₂Cl₂ with 4% CH₃OH and 1% ammonium hydroxide. The title compound was isolated as yellow film (25 mg; 45%). ¹H NMR (400 MHz, CDCl₃): 9.1 (s, 1H), 8.1 (s, 1H), 7.98 (s, 1H), 7.4 (s, 1H), 7.3 (s, 1H), 6.6 (s, 2H), 3.8 (t, 2H), 3.6 (s, 2H), 2.65 (s, 3H), 2.5 (br-s, 4H), 1.7 (br-s, 4H), 1.45 (br-s, 2H), 0.98 (t, 2H), 0.0 (s, 9H). HPLC-MS t_{R}=3.82 (UV₂₅₄ₙₘ). Mass Calc. for C₂₅H₃₅N₇OS₂Si 541.21; obsd MH⁺ (LCMS) 542.3 (m/z).

### Example 64

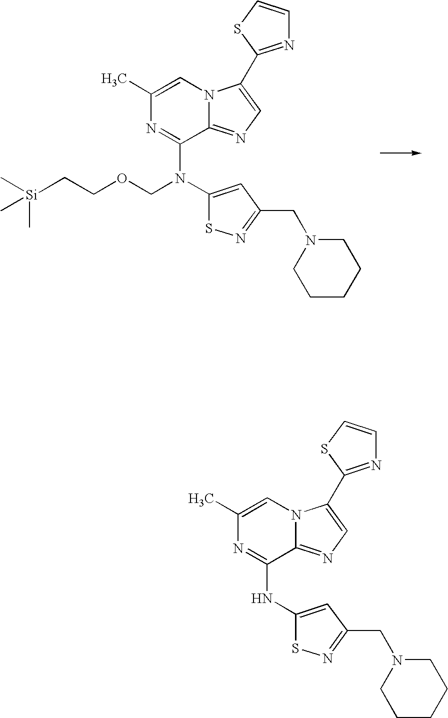

A solution of compound from Example 63 (0.013 g; 0.02 mmol) in 0.5 mL of THF was treated with HCl in dioxane (4M; 0.5 mL) and placed in an oil bath at 70° C. After heating for 30 min, a precipitate formed which dissolved upon adding 0.5 mL of methanol. The reaction mixture was heated at a bath temperature of 70° C. for an additional 1 hr. The contents of the reaction were cooled to RT and all the volatiles were removed on a rotary evaporator. The residue was suspended in THF and triturated with ether. The precipitate was collected by filtration, washed with ˜10 mL of ether and dried in air (0.5 hr) and in vacuo (16 hr) to furnish 10 mg (93%) of the title compound as a yellow solid. ¹H NMR (400 MHz, CD₃OD): 9.0 (s, 1H), 8.3 (s, 1H), 8.05 (s, 1H), 7.7 (s, 1H), 7.25 (s, 1H), 4.4 (s, 2H), 3.6 (d, 2H), 3.1 (t, 2H), 2.6 (s, 3H), 2.0 (d, 2H), 1.85 (t, 4H), 1.6 (t, 1H). HPLC-MS t_{R}=2.96 (UV₂₅₄ₙₘ). Mass Calc. for C₁₉H₂₁N₇S₂ 411.13; obsd MH⁺ (LCMS) 412.2 (m/z).

Compounds in the Table 9 (shown immediately below) were prepared as per the above-described examples:

TABLE 9

| Ex- | | | LCMS | |
|---|---|---|---|---|
| am- | | | MH⁺ | LCMS |
| ple | Structure | MW | m/z | MS t_{R} |
| 59-1 | 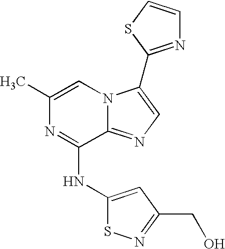 | 344.05 | 345.2 | 2.98 |
| 59-2 | 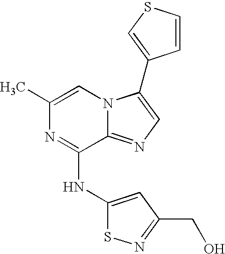 | 343.06 | 344.2 | 2.78 |
| 64-1 | 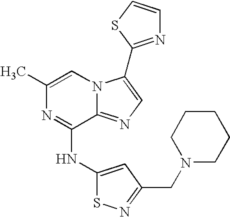 | 411.13 | 412.2 | 2.96 |
| 64-2 | 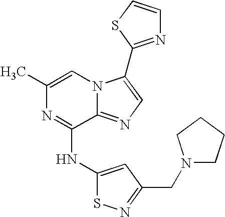 | 397.11 | 398.2 | 3.11 |
| 64-3 | 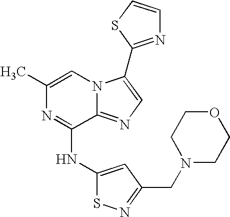 | 413.11 | 414.2 | 2.86 |
| 64-4 | 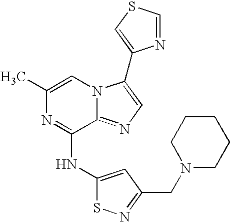 | 411.13 | 412.2 | 2.99 |
| 64-5 | 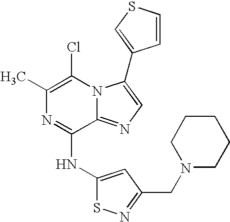 | 444.09 | 445.2 | 3.29 |

### Example 65

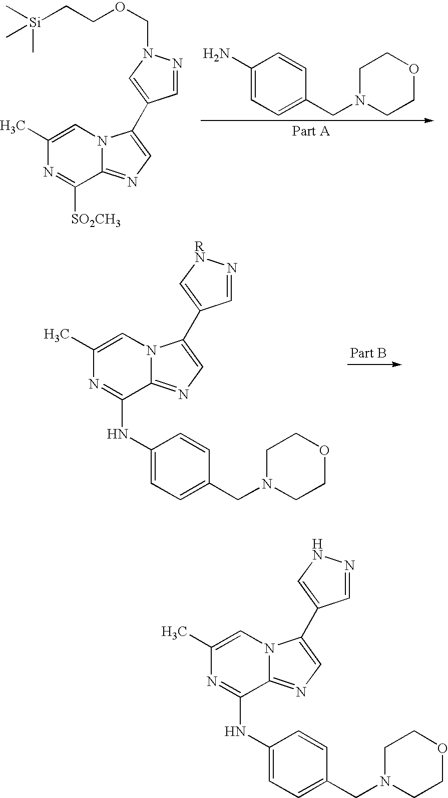

Part A: Lithium hexamethyldisilazide (1M in THF; 0.18 mL) was added to an amber solution of 4-morpholin-4-ylmethyl phenylamine (0.013 g; 0.068 mmol) and 8-methanesulfonyl-6-methyl-3-[1-(2-trimethylsilanyl-ethoxymethyl)-1H-pyrazol-4-yl]-imidazo[1, 2-α]pyrazine (0.025 g; 0.061 mmol) in 2 mL of THF at RT resulting in a burgundy solution. After stirring at RT for 20 minutes, the reaction mixture was quenched with saturated aqueous NH₄Cl solution. The contents were diluted with ethyl acetate and washed with water and brine. The crude material from the organic extract was purified by prep TLC (5% methanol-CH₂Cl₂) to obtain the title compound as pale yellow oil (0.025 g; 80%). NMR (400 MHz, CDCl₃): 8 (s, 1H), 7.9 (d, 2H), 7.85 (s, 1H), 7.8 (s, 1H), 7.5 (s, 1H), 7.4 (s, 1H), 7.35 (d, 2H), 5.55 (s, 2H), 3.75 (br-s, 4H), 3.7 (t, 2H), 3.5 (br-s, 2H), 2.5 (br-s, 2H), 2.4 (s, 3H), 1.6 (br-s, 2H), 0.95 (t, 2H), 0.0 (s, 9H). HPLC-MS t_{R}=3.05 (UV₂₅₄ₙₘ). Mass Calc. for C₂₇H₃₇N₇O₂Si 519.27; obsd MH⁺ (LCMS) 520.3 (m/z).

Part B: The compound from Part A (0.025 g; 0.048 mmol) was suspended in dry THF and treated with HCl in dioxane (4M; 1 mL) and heated in an oil bath set to 70° C. for 15 minutes when a white precipitate was formed. Methanol was added to dissolve some of the solid and the reaction mixture was continued to be heated for 45 minutes more. After cooling to RT, the volatiles were removed on the rotary evaporator. The residue was suspended in THF and the precipitated solid was collected by filtration, washed with ether and dried in vacuo overnight. The title compound was isolated as a beige solid (14 mg; 78%). All the analogues in Table 10 were similarly prepared.

TABLE 10

| | | | LCMS | |
|---|---|---|---|---|
| | | | MH⁺ | HPLC |
| Example | Structure | MW | (m/z) | MS t_{R} |
| 65-1 | 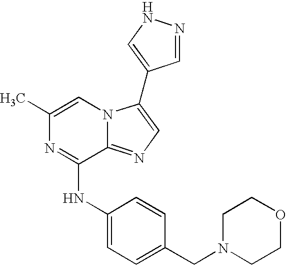 | 389.19 | 390.2 | 1.7 |
| 65-2 | 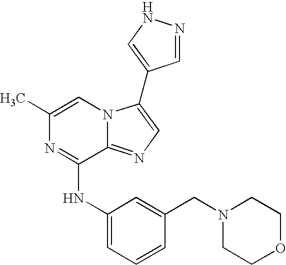 | 389.19 | 390.2 | 1.76 |
| 65-3 | 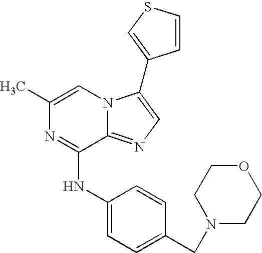 | 405.16 | 406.2 | 2.38 |
| 65-4 | 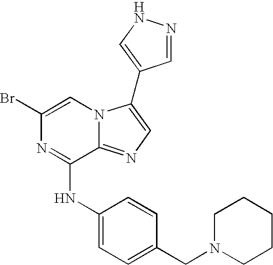 | 451.11 | 452.2 | 2.51 |
| 65-5 | 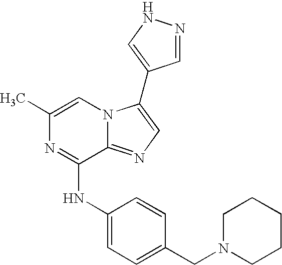 | 387.22 | 388.2 | 1.85 |
| 65-6 | 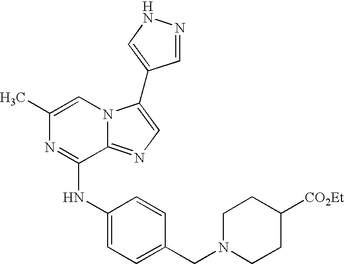 | 459.24 | 460.3 | 2.03 |
| 65-7 | 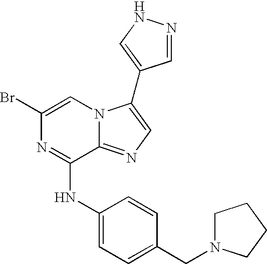 | 437.1 | 438.2 | 2.38 |
| 65-8 | 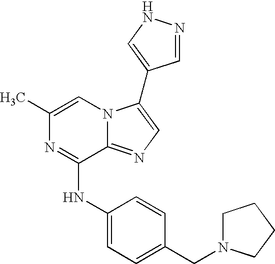 | 373.2 | 374.2 | 1.65 |
| 65-11 | 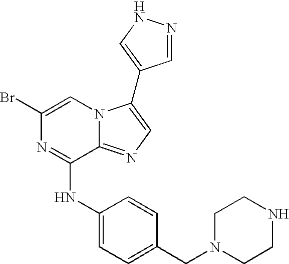 | 452.11 | 453.3 | 1.93 |
| 65-12 | 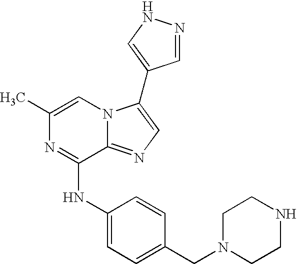 | 388.21 | 389.2 | 1.59 |
| 65-13 | 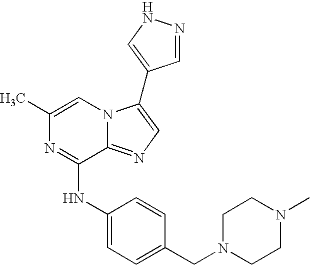 | 402.23 | 403.2 | 1.51 |
| 65-14 | 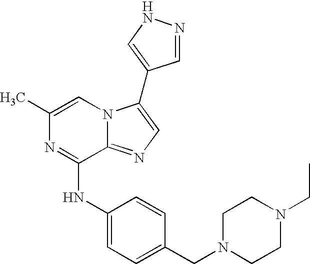 | 416.24 | 417.2 | 1.54 |
| 65-15 | 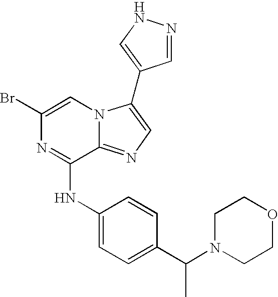 | 467.1 | 468.3 | 2.27 |
| 65-16 | 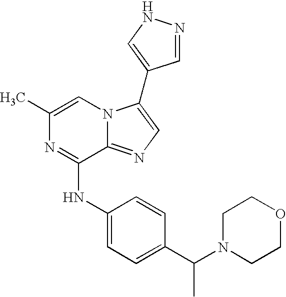 | 403.21 | 404.2 | 1.9 |
| 65-17 | 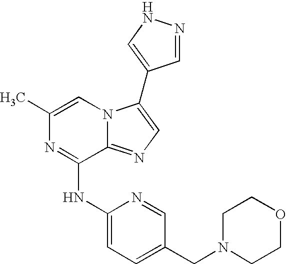 | 390.19 | 391.2 | 1.36 |

### Example 66

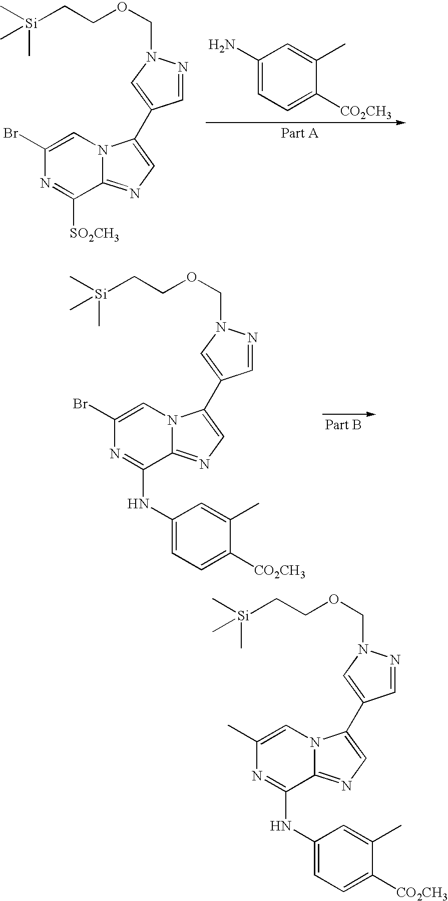

Part A: A solution of 4-Amino-2-methyl-benzoic acid methyl ester (0.33 g; 2 mmol; prepared from commercially available 4-nitro-2-methyl-benzoic acid) and 8-methanesulfonyl-6-bromo-3-[1-(2-trimethylsilanyl-ethoxymethyl)-1H-pyrazol-4-yl]-imidazo[1,2-a]pyrazine (0.472 g; 1.0 mmol) was treated with LiHMDS (1M in THF; 2 mL) at RT. The resulting burgundy solution was stirred at RT for 20 minutes and then quenched with saturated aqueous NH₄Cl solution. Standard work up as described for Example 65 and flash silicagel chromatography (25% EtOAc in CH₂Cl₂) provided the title compound as pale yellow foam (0.48 g; 86%). NMR (400 MHz, CDCl₃): 8.18 (s, 1H), 8 (d, 1H), 7.9 (s, 1H), 7.85 (d, 1H), 7.78 (s, 1H), 7.7 (s, 1H), 7.62 (s, 1H), 7.58 (s, 1H), 5.5 (s, 2H), 3.9 (s, 3H), 3.65 (t, 2H), 2.6 (s, 3H), 0.98 (t, 2H), 0.0 (s, 9H). Mass Calc. for C₂₄H₂₉BrN₆O₃Si 556.13; Obsd MH⁺ (CI-MS) 557/559 (m/z).

Part B: A solution of compound from Part A (0.48 g; 0.86 mmol) in 2 mL of dry THF was treated with a solution of dimethyl zinc (2M; 4 mL) dropwise. After the effervescence ceased, solid Pd(PPh₃)₄ was added and the reaction was flushed with nitrogen, fitted with a reflux condenser and heated in an oil bath at 65-70° C. After 0.5 hr, the reaction mixture had turned from yellow orange to deep red and after 4 more hours, it had become an opaque black. TLC (25% EtOAc-CH₂Cl₂) indicated the formation of a slightly more polar spot. The reaction was cooled to RT, quenched with saturated aqueous NH₄Cl solution and extracted with EtOAc. Flash silicagel chromatography of the crude material gave the 6-methyl title compound as yellow solid (0.38 g; 90%). NMR (400 MHz, CDCl₃): 8.1 (s, 1H), 8 (d, 1H), 7.9 (d, 1H), 7.85 (s, 1H), 7.8 (s, 1H), 7.7 (s, 1H), 7.58 (s, 1H), 7.4 (s, 1H), 5.5 (s, 2H), 3.9 (s, 3H), 3.65 (t, 2H), 2.65 (s, 3H), 2.4 (s, 3H), 0.98 (t, 2H), 0.0 (s, 9H). Mass Calc. for C₂₅H₃₂N₆O₃Si 492.23; Obsd MH⁺ (CI-MS) 493.11 (m/z).

### Example 67

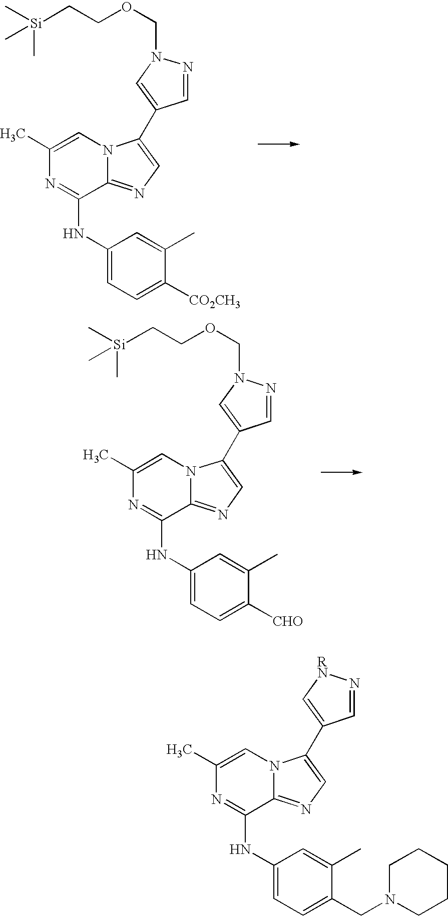

Part A: Ester was first reduced to the alcohol using LiBEt₃H in THF at RT and subsequently oxidized using Dess-Marin periodinane to the aldehyde as described previously. The reductive amination of aldehyde with various secondary amines was carried out provided SEM-protected title compound. Removal of the SEM protecting group was carried out under conditions described previously. In a similar manner, other tertiary amines listed in Table 11 (shown immediately below) were also prepared by the similar reaction scheme with the corresponding secondary amines followed by removal of the SEM protecting group.

TABLE 11

| | | | LCMS | |
|---|---|---|---|---|
| | | | MH⁺ | HPLC |
| Example | Structure | MW | (m/z) | MS t_{R} |
| 67-1 | 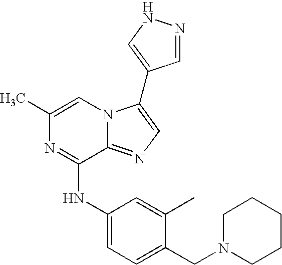 | 401.23 | 402.2 | 1.87 |
| 67-2 | 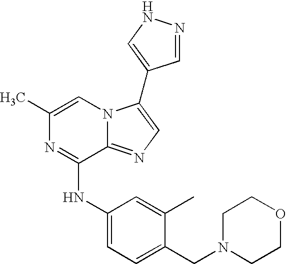 | 403.21 | 404.2 | 1.71 |
| 67-3 | 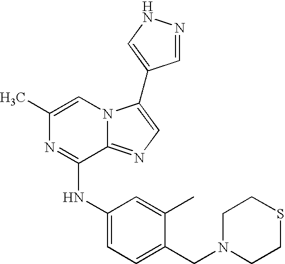 | 419.19 | 420.2 | 1.95 |
| 67-4 | 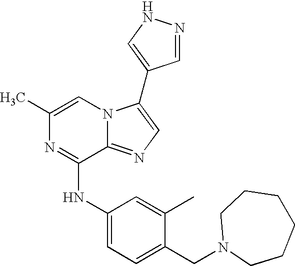 | 415.25 | 416.2 | 2.0 |

### Example 68

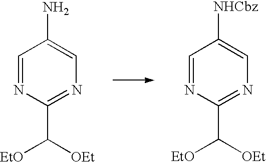

The substrate (1 g, 5.07 mmol) was dissolved in THF:H₂O (12 mL, 1:1, v/v) and treated with K₂CO₃ (1.4 g, 10.15 mmol) at room temperature. Then benzyl chloroformate (0.79 ml, 5.58 mmol) in THF (2 mL) was slowly added. The mixture was stirred for 16 h. It was diluted with ethyl acetate (25 mL). The two layers were separated, and the aqueous layer was extracted with ethyl acetate (2×25 mL). The combined organic layer was washed with brine (1×30 mL), dried (Na₂SO₄), filtered and evaporated under reduced pressure to give the crude product which was purified by column chromatography (SiO₂) eluting ethyl acetate-hexane.

### Example 69

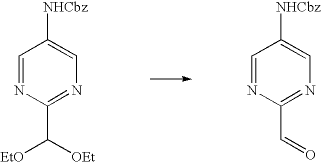

The substrate acetal (1.2 g, 3.64 g) was dissolved in acetone (20 mL), and treated with 1N aqueous HCl (2 mL) at room temperature, and the mixture was stirred for 7 h. Then acetone was evaporated off, and the residue was diluted with saturated aqueous NaHCO₃ (30 mL). The aqueous layer was extracted with ethyl acetate (2×30 mL). The combined organic layer was washed with brine (1×30 mL), dried (Na₂SO₄), filtered and evaporated under reduced pressure to give the crude product as solid which was used in the next step without any further purification.

### Example 70

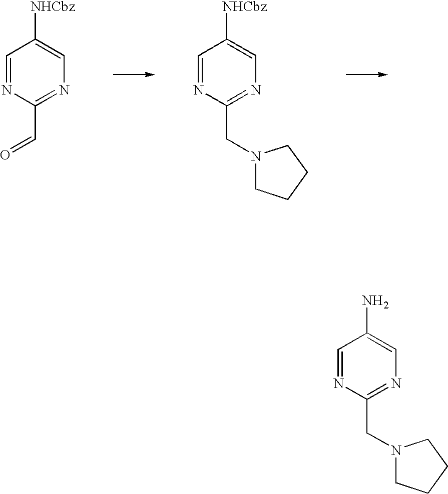

The substrate (1 eq.), amine (4 eq.), catalytic AcOH, NaB(OAc)₃H (2 eq.) in 1,2-dichloroethane was stirred at room temperature for 2 h. Then sodium borohydride (3 eq.) was added and the mixture was stirred for 30 min at which point LC-MS analysis indicate complete consumption of starting material to product. Then the reaction was quenched with 2N aqueous NaOH, and the mixture was stirred vigorously until two clear layer separated. The organic layer was washed with water, brine, dried (Na₂SO₄), filtered and concentrated under reduced pressure to give the product.

The crude product was hydrogenated in ethyl acetate using 10% Pd/C at 1 atmosphere hydrogen pressure. The catalyst was filtered off, and solvent was evaporated under reduce pressure to give the crude product.

### Example 71

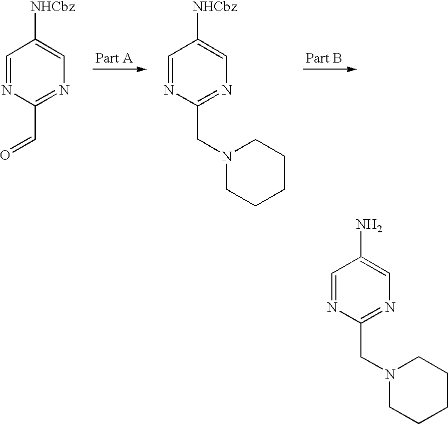

Part A: The substrate (1 eq.), amine (4 eq.), catalytic AcOH, NaB(OAc)₃H in 1,2-dichloroethane was stirred at room temperature for 2 h. Then sodium borohydride (3 eq.) was added and the mixture was stirred for 30 min at which point LC-MS analysis indicate complete consumption of starting material to product. Then the reaction was quenched with 2N aqueous NaOH, and the mixture was stirred vigorously until two clear layer separated. The organic layer was washed with water, brine, dried (Na₂SO₄), filtered and concentrated under reduced pressure to give the product.

Part B: The crude product was hydrogenated in ethyl acetate using 10% Pd/C at 1 atmosphere hydrogen pressure. The catalyst was filtered off, and solvent was evaporated under reduce pressure to give the crude product.

### Example 72

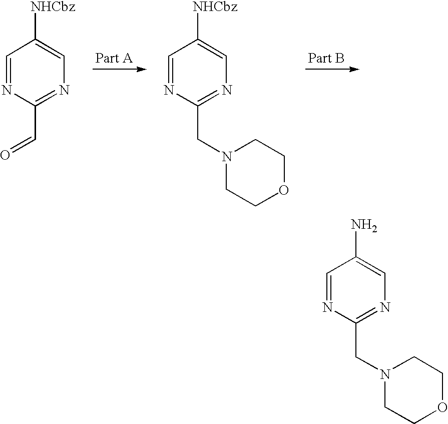

Part A: The substrate (1 eq.), amine (4 eq.), catalytic AcOH, NaB(OAc)₃H in 1,2-dichloroethane was stirred at room temperature for 2 h. Then sodium borohydride (3 eq.) was added and the mixture was stirred for 30 min at which point LC-MS analysis indicate complete consumption of starting material to product. Then the reaction was quenched with 2N aqueous NaOH, and the mixture was stirred vigorously until two clear layer separated. The organic layer was washed with water, brine, dried (Na₂SO₄), filtered and concentrated under reduced pressure to give the product.

Part B: The crude product was hydrogenated in ethyl acetate using 10% Pd/C at 1 atmosphere hydrogen pressure. The catalyst was filtered off, and solvent was evaporated under reduce pressure to give the crude product.

### Example 73

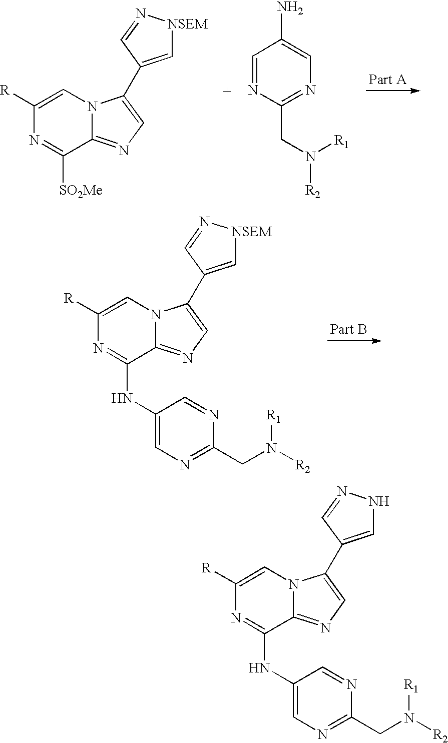

Part A: The substrate (1 eq.) and amine (1.5-2 eq.) was dissolved in DMSO under argon, and treated with NaH (5 eq., 60% dispersion in oil). After 30 min, LC-MS analysis indicated complete consumption of starting material. The reaction was quenched by addition of saturated aqueous NH₄Cl-acetonitrile (1:1, v/v). The two layers were separated, and the aqueous layer was extracted with ethyl acetate. The combined organic layer was washed with brine, dried (Na₂SO₄), filtered and concentrated under reduced pressure to give the crude product.

Part B: The substrate was dissolved in 4N HCl in dioxane, and stirred at room temperature for 30 min. The solvent was then evaporated, and the residue was purified by Prep-LC. Conversion to hydrochloride salt afforded the product as solid. The compounds in Table 12 (shown immediately below) were prepared accordingly:

TABLE 12

| | | | LCMS | HPLC MS |
|---|---|---|---|---|
| Example | Column | MW | MH⁺ m/z | t_{R} |
| 73-1 | 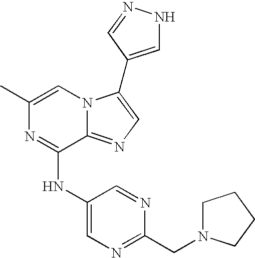 | 375.4 | 376.1 | 0.83 |
| 73-2 | 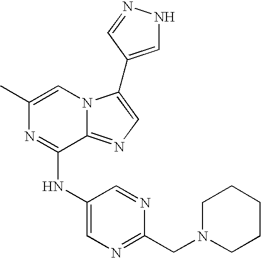 | 389.4 | 390.2 | 1.05 |
| 73-3 | 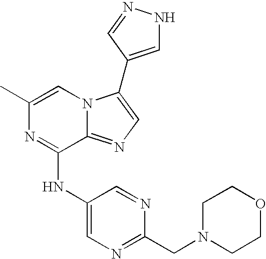 | 391.4 | 392.1 | 0.78 |
| 73-4 | 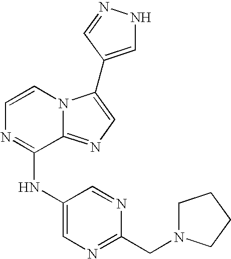 | 361.4 | 362.2 | 0.74 |
| 73-5 | 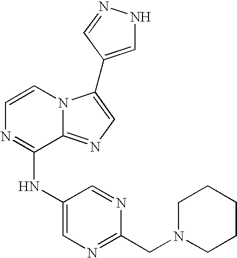 | 375.4 | 376.2 | 0.85 |
| 73-6 | 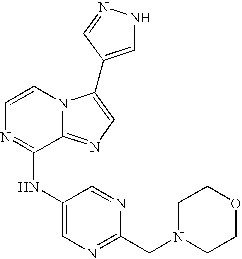 | 377.4 | 378.3 | 0.87 |

### Example 74

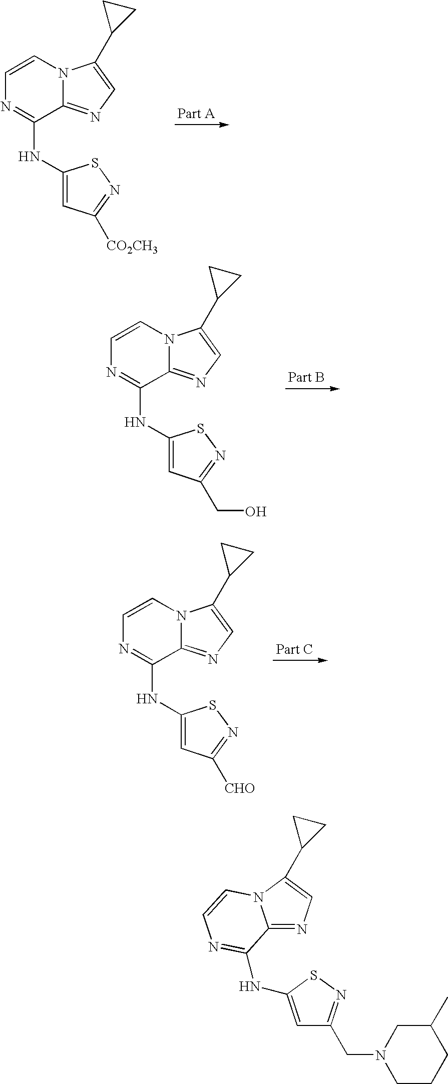

Part A: By essentially the same procedure as described for Example 1 and 13.

Part B: To a solution of compound from Example 74, Part A (0.16 g, 0.57 mmol) in THF (20 mL) and water (0.025 mL) was added Dess-Martin periodinane (3 equivalents). The resulting solution was stirred at rt for 1.5 h at which time LC-MS analysis indicated the reaction was complete. The reaction mixture was diluted with dichloromethane (75 mL), washed with water, dried (sodium sulfate) and concentrated. Purification by column chromatography (SiO₂ 10% methanol/DCM) afforded the title compound as a yellow solid 0.08 g (49%). HPLC-MS t_{R}=1.59 Min (UV₂₅₄ₙₘ). Mass calculated for formula C13H11N5OS 285.07, observed LC/MS m/z 286.1 (M+H).

Part C: To a solution of compound from Part B (30 mg, 0.105 mmol, 1 equivalent), 3-methylpiperidine (10 equivalents) in dichloromethane:methanol (5:1) (3 ml) was added acetic acid (1 drop). The resulting solution was stirred at rt for 30 minutes, and then sodium borohydride (8 equivalents) added to the reaction. The reaction mixture was stirred at rt for 1 hour at which time LC-MS analysis indicated the reaction was complete. The reaction was quenched with sat. aq. sodium bicarbonate and then extracted with dichloromethane (×2). The combined organic layer was dried (sodium sulfate) and concentrated. Purification by Prep-LC and conversion to a hydrochloric salt afforded the title compound. HPLC-MS t_{R}=3.73 Min (UV₂₅₄ₙₘ). Mass calculated for formula C₁₉H₂₄N₆S 368.18, observed LC/MS m/z 369.2 (M+H).

### Example 75

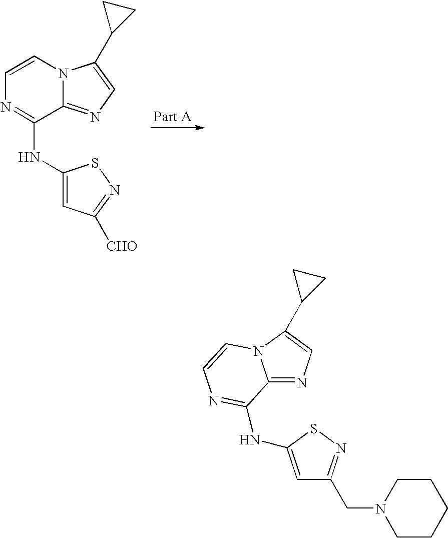

Part A: To a solution of compound 1 (30 mg, 0.105 mmol, 1 equivalent), piperidine (10 equivalents) in dichloromethane:methanol (5:1) (3 ml) was added acetic acid (1 drop). The resulting solution was stirred at rt for 30 minutes, and then sodium borohydride (8 equivalents) added to the reaction. The reaction mixture was stirred at rt for 1 h at which time LC-MS analysis indicated the reaction was complete. The reaction was quenched with sat. aq. sodium bicarbonate and then extracted with dichloromethane (×2). The combined organic layer was dried (sodium sulfate) and concentrated. Purification by Prep-LC and conversion to a hydrochloric salt afforded compound 2. HPLC-MS t_{R}=3.47 Min (UV₂₅₄ₘₙ). Mass calculated for formula C₁₈H₂₂N₆S 354.16, observed LC/MS m/z 355.1 (M+H).

### Example 76

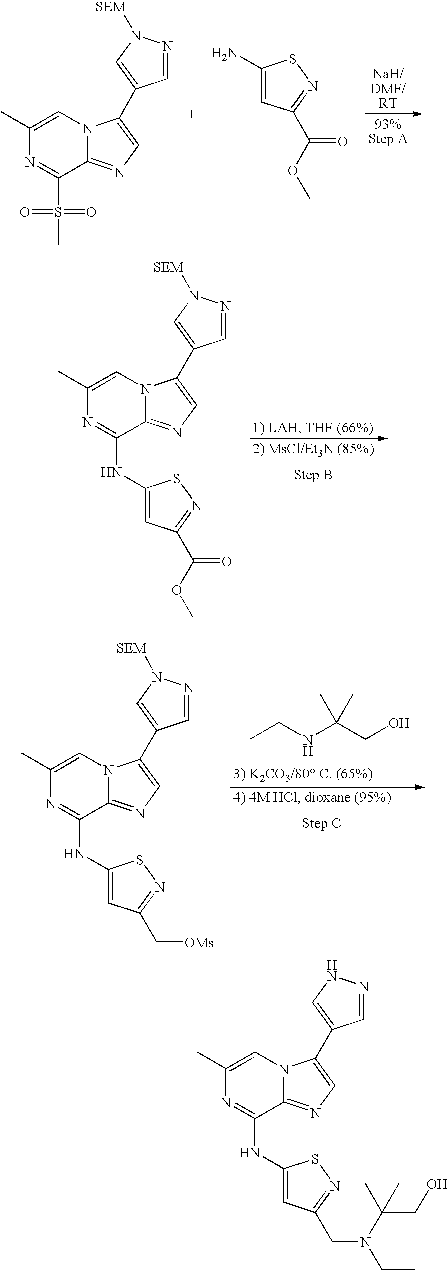

Step A: Sodium hydride (60% dispersion in mineral oil, 6.68 g, 3.40 equiv) was slowly added in one portion to a stirring mixture of compound sulfone (20.0 g, 1.00 equiv) and aminoisothiazole (11.5 g, 1.20 equiv, as HCl salt) in DMF (490 mL) at room temperature (with aid of a room temperature water bath). Reaction was allowed to stir for 1 hour at which time HPLC analysis indicated the reaction was complete. The reaction was carefully quenched with saturated aqueous sodium bicarbonate (200 mL) and then diluted with water (1 L). This mixture was stirred for 20 minutes at room temperature, and then the resulting precipitate was collected by filtration, washed with water (200 mL), and dried under high vacuum for 16 hours. The resulting waxy solid was dissolved in 1.8 L of 1:1 chloroform:methanol, dried over sodium sulfate, filtered, and concentrated under reduced pressure to give the title compound (22.3 g, 93%) as a dark yellow solid. ¹H NMR (300 MHz, DMSO-d₆) δ 12.3 (bs, 1H), 8.60 (s, 1H), 8.10 (s, 1H), 7.88 (s, 2H), 7.59 (s, 1H), 5.51 (s, 2H), 3.85 (s, 3H), 3.63 (d, J=8 Hz, 2H), 2.48 (s, 3H), 0.88 (d, J=8 Hz, 2H), −0.026 (s, 9H). Mass calculated for formula C₂₁H₂₇N₇O₃SSi 485.63; observed MH⁺ (MS) 486.6 (m/z).

Step B: A mixture of compound from Step A (4.27 g, 3.73 mmol) was dissolved in 180 mL of THF. The resulting solution was cooled to 0° C. and LiAlH₄ powder (2.6 g, 68.5 mmol) was carefully added. The cooling bath was removed and the reaction was stirred at RT under a N₂ atmosphere for 1.5 hr. The reaction was cooled to 0° C. and carefully quenched by the sequential addition of 2.6 mL of H₂O; 2.6 mL of 15% NaOH (aq); 7.8 mL H₂O. After stirring for 10 min, the reaction was filtered through a very thin pad of Celite (rinsing with THF, EtOAc and DCM). Concentration of the filtrate yielded a light yellow solid. Pure alcohol (2.66 g, 66% yield) was obtained via triturating with MeOH and used directly in the next step.

Step C: A mixture of compound from Step B (2.40 g, 4.49 mmol), amine (1.57 g, 13.46 mmol), and NaI (63.0 mg, 0.449 mmol) in 45 mL of THF was heated at 80° C. for 12 h. It was diluted with 200 mL of CH₂Cl₂, and washed with 100 mL of saturated aqueous NaHCO₃ solution, then with brine (100 mL). The solvent was removed under vacuum. The residue was purified by flash chromatography eluting with 5% to 10% MeOH/CH₂Cl₂ to give 1.68 g of the title compound. ¹H NMR (400 MHz, CDCl₃) δ 9.49 (brs, 1H), 7.89 (s, 1H), 7.82 (s, 1H), 7.60 (s, 1H), 7.49 (s, 1H), 6.86 (s, 1H), 5.54 (s, 2H), 3.79 (brs, 3H), 3.67 (t, J=8.3 Hz, 2H), 3.36 (s, 2H), 2.65-2.80 (m, 2H), 2.50 (s, 3H), 1.11 (s, 6H), 1.02 (t, J=7.1 Hz, 2H), 0.96 (t, J=8.2 Hz, 2H), 0.01 (s, 9H). To a solution of Sem-protected compound (2.0 g, 3.6 mmol) in 36 mL of MeOH/CH₂Cl₂ (1:1) stirred at 80° C., was added 36 mL of 4 N HCl in dioxane. The reaction was stirred at 80° C. for 30 min. After it was cooled to the room temperature, 120 mL of ether was added. The solid was collected by filtration, washed with ether and dried under vacuum to give 2.0 g of the title compound as its HCl salt form. Mass calculated for formula C₂₀H₂₆N₈OS 426.2; observed MH⁺ (LCMS) 427.2 (m/z).

Using essentially the same procedures as described for Example 76, the following compounds in Table 13 (shown immediately below) were prepared.

TABLE 13

| | | | LCMS | |
|---|---|---|---|---|
| | | | MH⁺ | HPLC |
| Example | Column 2 | EM | m/z | MS t_{R} |
| 76-1 | 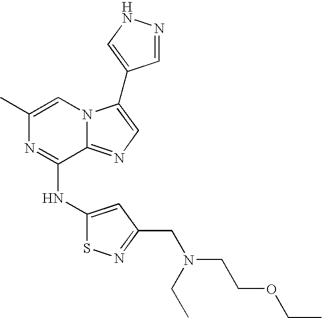 | 426.2 | 427.2 | 2.28 |
| 76-2 | 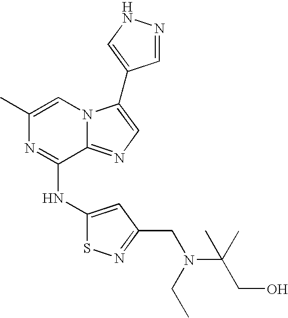 | 426.2 | 427.2 | 2.21 |
| 76-3 | 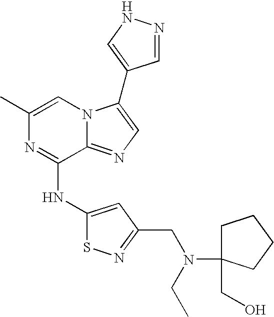 | 452.2 | 453.2 | 2.26 |
| 76-4 | 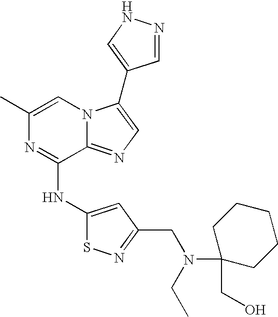 | 466.3 | 467.3 | 2.48 |
| 76-5 | 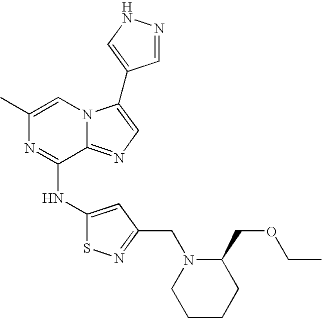 | 452.2 | 453.2 | 2.35 |
| 76-6 | 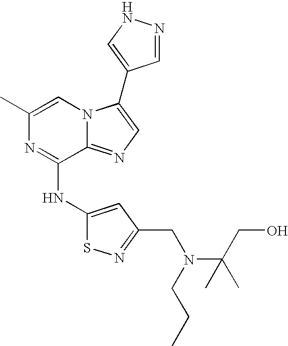 | 440.2 | 441.2 | 2.39 |
| 76-7 | 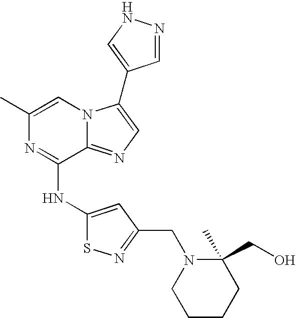 | 438.1 | 439.1 | 1.81 |
| 76-8 | 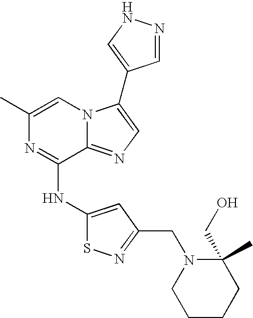 | 438.1 | 439.1 | 1.03 |
| 76-9 | 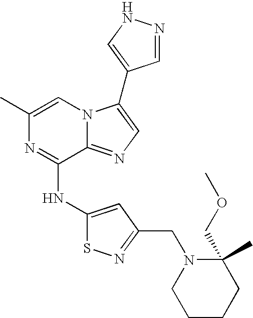 | 452.2 | 453.3 | 1.48 |
| 76-10 | 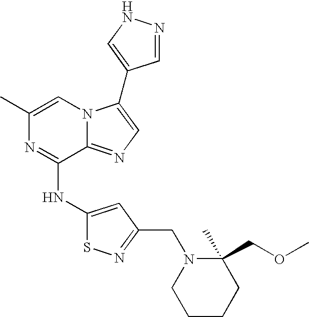 | 452.2 | 453.1 | 1.48 |
| 76-11 | 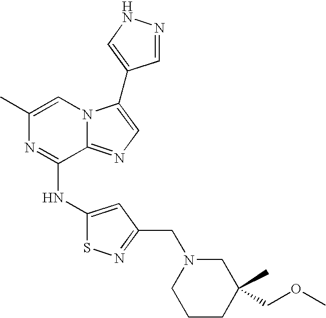 | 452.2 | 453.3 | 1.35 |
| 76-12 | 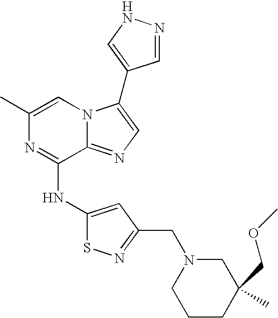 | 452.2 | 453.2 | 1.36 |

### Example 76-1

¹H NMR (400 MHz, CD₃OD) δ 8.24 (s, 1H), 8.23 (s, 2H), 8.07 (s, 1H), 7.35 (s, 1H), 4.57 (d, J=12.8 Hz, 2H), 3.81 (t, J=4.8, 2H), 3.57 (q, J=14.0, 6.8 Hz, 2H), 3.52 (m, 2H), 3.41 (m, 2H), 2.60 (s, 3H), 1.38 (t, J=7.2 Hz, 3H), 1.21 (t, J=6.8 Hz, 3H). HPLC-MS t_{R}=2.28 min (UV₂₅₄ₙₘ). Mass calculated for formula C₂₀H₂₆N₈OS 426.2; observed MH⁺ (LCMS) 427.2 (m/z).

### Example 76-2

¹H NMR (400 MHz, CD₃OD) δ 8.31 (s, 1H), 8.29 (s, 2H), 7.32 (s, 1H), 4.88 (d, 1H), 4.46 (d, J=16.1 Hz, 1H), 3.82 (d, J=12.3 Hz, 1H), 3.71 (d, J=12.3 Hz, 1H), 3.64 (m, 1H), 2.65 (s, 3H), 1.42 (s, 3H), 1.40 (s, 3H), 1.31 (t, J=7.1 Hz, 3H). HPLC-MS t_{R}=2.26 min (UV₂₅₄ₙₘ).

### Example 76-3

¹H NMR (400 MHz, CD₃OD) δ 8.16 (s, 2H), 8.13 (s, 1H), 7.99 (s, 1H), 7.25 (s, 1H), 4.72 (d, J=15.6 Hz, 1H), 4.53 (t, J=15.6, 1H), 3.66 (s, 2H), 3.61 (m, 1H), 3.40 (m, 1H), 2.57 (s, 3H), 1.33-0.95 (8H), 1.17 (t, J=6.8 Hz, 3H). HPLC-MS t_{R}=2.26 min (UV₂₅₄ₙₘ). Mass calculated for formula C₂₀H₂₆N₈OS 452.2; observed MH⁺ (LCMS) 453.2 (m/z).

### Example 76-4

¹H NMR (400 MHz, CD₃OD) δ 8.16 (s, 2H), 8.15 (s, 1H), 8.00 (s, 1H), 7.21 (s, 1H), 4.95 (d, J=16.0 Hz, 1H), 4.35 (d, J=16.8 Hz, 1H), 4.07 (d, J=12.4 Hz 1H), 3.82 (d, J=12.8 Hz 1H), 3.52 (m, 2H), 2.57 (s, 3H), 1.96-1.58 (10H), 1.26 (t, J=6.8 Hz, 3H). HPLC-MS t_{R}=2.48 min (UV₂₅₄ₙₘ). Mass calculated for formula C₂₀H₂₆N₈OS 466.3; observed MH⁺(LCMS) 467.3 (m/z).

### Example 76-5

¹H NMR (400 MHz, CD₃OD) δ 8.28 (s, 1H), 8.25 (s, 2H), 8.10 (s, 1H), 7.39 (s, 1H), 4.65 (d, J=14.0 Hz, 1H), 4.52 (d, J=10.8 Hz, 1H), 4.24 (d, J=14.0 Hz 1H), 3.85 (d, J=18.0 Hz 1H), 3.65-3.44 (4H), 2.65 (s, 3H), 1.97-1.58 (6H), 1.29 (t, J=7.2 Hz, 3H). HPLC-MS t_{R}=2.35 min (UV₂₅₄ₙₘ). Mass calculated for formula C₂₀H₂₆N₈OS 452.2; observed MH⁺ (LCMS) 453.2 (m/z).

### Example 76-6

¹H NMR (400 MHz, CD₃OD) δ 8.21 (s, 3H), 8.03 (s, 1H), 7.23 (s, 1H), 4.38 (d, J=5.6 Hz, 1H), 3.79 (d, J=5.4 Hz, 1H), 3.63 (d, J=12.4 Hz 1H), 3.53 (m, 1H), 3.10 (m, 1H), 2.58 (s, 3H), 1.34 (s, 6H), 0.87 (t, J=6.2 Hz, 3H). HPLC-MS t_{R}=2.39 min (UV₂₅₄ₙₘ). Mass calculated for formula C₂₀H₂₆N₈OS 440.2; observed MH⁺ (LCMS) 441.2 (m/z).

### Example 76-7

¹H-NMR (400 MHz, CD₃OD) δ 8.33 m (3H), 8.15 s (1H), 7.41 s (1H), 4.80 (d, 2H), 4.15 (d, 2H), 4.06 (d, 2H), 3.62 (d, 2H), 3.58 (m, 1H), 2.68 (d, 3H), 2.21 (m, 1H), 1.81 (m, 6H) and 1.45 (s, 3H). HPLC-MS t_{R}=1.80 Min (UV₂₅₄ₙₘ). Mass calculated for formula C21H26N8OS 438.55, observed LC/MS m/z 439.1 (M+H).

### Example 76-8

¹H-NMR (400 MHz, DMSO-d₆) δ 12.73 bs (1H), 9.2 bs (1H), 8.28 s (2H), 8.09 s (1H), 8.08 s (1H), 7.36 s (1H), 4.71 m (1H), 4.05 m (1H), 3.82 m (1H), 3.63 m (1H), 3.25 m (2H), 1.97 m (1H), 1.65 m (6H) and 1.30 s (3H).

### Example 76-9

¹H-NMR (400 MHz, DMSO-d₆) δ 8.28(1H), 8.25 (2H), 8.08 (1H), 7.32 (1H), 4.71 (1H), 4.08 (1H), 3.84 (1H), 3.52 (3H), 3.46 (1H), 2.63 (3H), 2.17 (2H), 1.87-1.73 (6

H), 1.45 (3H).

### Example 76-10

¹H-NMR (400 MHz, DMSO-d₆) δ 8.28 (1H), 8.25 (2H), 8.08 (1H), 7.32 (1H), 4.71 (1H), 4.08 (1H), 3.84 (1H), 3.52 (3H), 3.46 (1H), 2.63 (3H), 2.17 (2H), 1.87-1.73 (6 μl), 1.45 (3H).

### Example 76-11

¹HNMR (400 MHz, CD₃OD) δ 8.20 (s, 2H), 8.14 (s, 1H), 8.03 (s, 1H), 7.25 (s, 1H), 4.48 (d, 1H), 4.37 (d, 1H), 3.46 (s, 3H), 2.91-3.60 (m, 6H), 2.62 (s, 3H), 1.40-1.89 (m, 4H), 0.92 (s, 3H).

### Example 76-12

¹HNMR (400 MHz, CD₃OD) δ 8.20 (s, 2H), 8.14 (s, 1H), 8.03 (s, 1H), 7.25 (s, 1H), 4.48 (d, 1H), 4.37 (d, 1H), 3.46 (s, 3H), 2.91-3.60 (m, 6H), 2.62 (s, 3H), 1.40-1.89 (m, 4H), 0.92 (s, 3H).

### Example 77

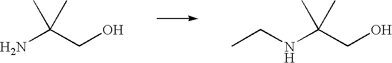

A mixture of iodoethane (52.5 g, 336.5 mmol) and 2-amino-2-methyl-1-propanol (30.0 g, 336.5 mmol) was stirred at 60° C. for 15 min. It was diluted with 500 mL of ether, and basified by adding 5 N aqueous NaOH until it reaches pH=10. The organic layer was separated. The aqueous layer was extracted with ether (500 mL×3). The combined organic was washed sequentially with 100 mL of water, and 100 mL of brine, then dried over anhydrous Na₂SO₄. The solvent was removed to provide 20 g of the crude product, which was purified by recrystallization in 150 mL of hexanes to give 13 g of a white solid. The solid was further purified by sublimation under reduced pressure to give 12 g of the title compound. ¹H NMR (400 MHz, CDCl₃) δ 3.28 (s, 2H), 2.54 (qt, J=7.1 Hz, 2H), 1.09 (t, J=7.0 Hz, 3H), 1.07 (s, 6H).

### Example 78

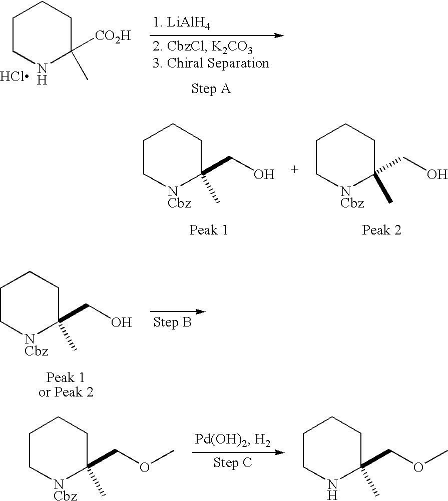

Step A: The substrate (10 g) was suspended in THF (200 mL). Then lithium aluminum hydride solution (110 mL, 2M in THF) was slowly added. The mixture was stirred at room temperature for 12 h. The solution was cooled to 0° C., and saturated aqueous Na₂SO₄ (200 mL) was slowly added. The mixture was filtered through Celite, and filter cake was washed with ethyl acetate (400 mL). The organic layer was washed with water (200 mL) and brine (200 mL). The organic layer was dried (anhydrous Na₂SO₄), filtered and evaporated to give the amino alcohol (6.9 g). The amino alcohol (6.9 g) was dissolved in THF (80 mL) and water (80 mL) at room temperature. The potassium carbonate (14.76 g) was added. Then benzyl chloroformate (8.28 mL) in THF (40 mL) was added dropwise. The mixture was stirred at room temperature for 30 min. Solvent was evaporated off under reduced pressure, and ethyl acetate (100 mL) was added. Two layers were separated, and the aqueous layer was extracted with ethyl acetate (2×100 mL). The combined organic layer was washed with brine (200 mL), dried (Na₂SO₄), filtered and evaporated under reduced pressure to give the crude product which was purified by column chromatography. The racemic aminoalcohol was chirally separated by SFC HPCL method. Then enantiomers corresponding to peak 1 and peak 2 were separately taken forward to prepare the corresponding building blocks.

Step B: The alcohol from Step A (1.936 g) was dissolved in dichloromethane (80 mL), and treated with proton sponge (8.32 g) at room temperature. Then trimethyloxonium tetrafluoroborate (5.69 g) was added. The mixture was stirred for 1 h. The reaction was quenched with saturated aqueous ammonium chloride solution (100 mL). The two layers were separated, and the aqueous layer was extracted with dichloromethane (2×100 mL). The combined organic layer was washed sequentially with hydrochloric acid (200 mL, 1 N), saturated sodium bicarbonate solution (200 mL), brine (200 mL), dried (Na₂SO₄), filtered and evaporated under reduced pressure to give the crude product which was purified by column chromatography.

Step C: The enantiomerically pure methyl ether from Step B in EtOH was treated with Pd(OH)₂ on carbon (20% wt) and stirred in hydrogen atmosphere at atmospheric pressure at room temperature for 2 h. The mixture was filtered off, and the filtrate was evaporated under reduced pressure to give the amine.

### Example 79

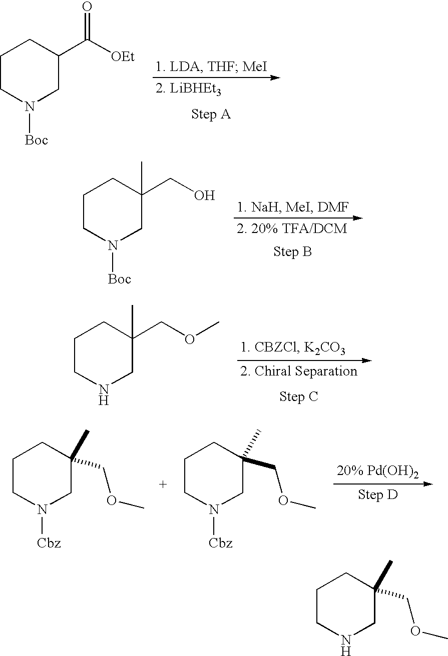

Step A: At −78° C., ester (6359 mg, 24.7 mmol) in THF (50 ml) was added dropwise to LDA (1.8 M in THF, 27.5 ml, 49.4 mmol) in THF (200 ml). The reaction mixture was slowly warmed up to room temperature and stirred at that temperature overnight. The reaction was cooled to 0° C. and quenched with saturated NH₄Cl solution. The mixture was diluted with H₂O and extracted with EtOAc (×2). The combined organic layer was dried over anhydrous Na₂SO₄ and concentrated. Purification by column chromatography afforded the title compound (6221 mg, 93%). LCMS t_{R}=2.27 Min. Mass calculated for, M+ 271.1, observed LC/MS m/z 216.1 (M+H—C₄H₈). To a solution of ester (4659 mg, 17.2 mmol) in THF (300 ml) was added LiBHEt₃ (69 ml, 1M in THF). The reaction was stirred at room temperature for 30 min. It was quenched by adding saturated NH₄Cl. The mixture was extracted with CH₂Cl₂. The combined organic layer was dried over anhydrous Na₂SO₄ and concentrated. Purification by column chromatography afforded the title compound (3032 mg, 77%). LCMS t_{R}=1.82 Min. Mass calculated for, M+ 229.1, observed LC/MS m/z 174.1 (M+H—C₄H₈).

Step B: NaH (1324 mg, 60% dispersion in mineral oil, 33.1 mmol) was added portion wise to a mixture of compound from Step A and MeI (3.3 ml, 52.9 mmol) in DMF (66 mL) at 0° C. The reaction mixture was slowly warmed up to room temperature and stirred at that temperature overnight. The reaction was cooled to 0° C. and quenched with saturated NH₄Cl solution. The mixture was diluted with H₂O and extracted with EtOAc (×2). The combined organic layer was dried over anhydrous Na₂SO₄ and concentrated. Purification by column chromatography afforded the title compound (2633 mg, 82%). LCMS t_{R}=2.32 Min. Mass calculated for, M+243.1, observed LC/MS m/z 188.1 (M+H—C₄H₈). A solution of compound 4 (901 mg, 3.71 mmol) was stirred in 20% TFA in CH₂Cl₂ (20 mL) at 0° C. for 30 min and then room temperature for 15 min. The reaction mixture was concentrated. The crude residue was used for example without further purification. LCMS t_{R}=0.26 Min. Mass calculated for, M+ 143.1, observed LC/MS m/z 144.1 (M+H).

Step C: CbzCl (604 μl, 4.08 mmol) in THF (1 ml) was added to a mixture of compound from Step B and K₂CO3 (1125 mg, 8.15 mmol) in THF (20 ml) and H₂O (20 ml) at 0° C. After stirring at room temperature for 30 min, the reaction mixture was extracted with EtOAc (×2). The combined organic layer was dried over anhydrous Na₂SO₄ and concentrated. Purification by column chromatography afforded the title compound (965 mg, 94%). LCMS t_{R}=2.28 Min (UV₂₅₄ₙₘ). Mass calculated for, M+ 277.1, observed LC/MS m/z 278.1 (M+H). The title amines were chirally separated utilizing a Gilson GX-281 liquid handling system with HPLC capabilities. Separation was accomplished with the following conditions: Chiral Technologies Chiral PAK AD column (5×50 cm; 20μ); flow=50 mL/min; 7.5% isopropanol in hexanes (isocratic); observed at 210 nm.

Step D: The enantiomerically pure isomers from Step C were dissolved in (1 mmol, 277 mg) in EtOH (6 ml) was mixed with 20% Pd(OH)₂ (51 mg) and stirred under H₂ balloon at room temperature for 2 h. Filtration through celite and concentration afforded the title compound, which was used for next step without further purification. LCMS t_{R}=0.26 Min. Mass calculated for, M+ 143.1, observed LC/MS m/z 144.1 (M+H).

The synthesis of the compounds in paragraph (j), as reported in U.S. provisional patent application Ser. No. 60/855,421, is shown below:

### Example 1,

### Preparation of Intermediate Compound JA

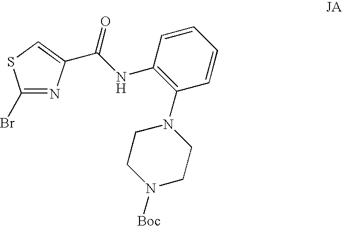

To a solution of 2-bromo-thiazole-4-carboxylic acid (2.0 mmol, 0.42 g), N,N-diisopropylethylamine (3.0 mmol, 0.52 mL) and HATU (2.0 mmol, 0.76 g) in DMF (10 mL) was added 4-(2-aminophenyl)-piperazine-1-carboxylic acid tert-butyl ester (2.0 mmol, 0.56 g). The reaction mixture was stirred at 80° C. for 3 h, and then concentrated in vacuo. The resulting residue was purified using flash column chromatography on silica gel (eluent: Hexane:EtOAc (4.5:1)) to provide Compound JA as a yellow solid (0.67 g, 72%). ¹H NMR (400 MHz, CDCl₃) □ 10.38 (s, 1H), 8.49 (dd, J=8.0, 1.2 Hz, 1H), 8.14 (s, 1H), 7.23-7.10 (m, 3H), 3.72 (br s, 4H), 2.89-2.87 (m, 4H), 1.50 (s, 9H). HPLC-MS RT=2.39 min, mass calculated for formula C₁₉H₂₃BrN₄O₃S 466.07, observed LCMS m/z 467.05 (M+H).

### Example 2

### Preparation of Compound 1

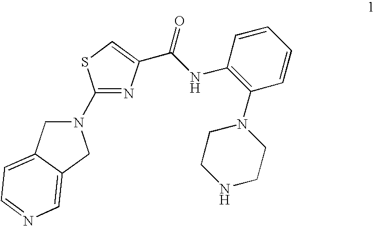

A solution of Compound JA (0.050 mmol, 23 mg), N,N-diisopropylethylamine (0.20 mmol, 35 □L) and 2,3-dihydro-1H-pyrrolo[3,4-c]pyridine (0.1 mmol) in DMF (1 mL) was irradiated using microwave for 15 minutes at a temperature of 180° C. The reaction mixture was then concentrated in vacuo, and to the resulting residue was added TFA (0.5 mL). The resulting solution was allowed to stir at room temperature for 10 minutes and was then concentrated in vacuo. The resulting residue was purified using reverse phase HPLC to provide Compound 1.

### Example 3

### Preparation of Compound 2

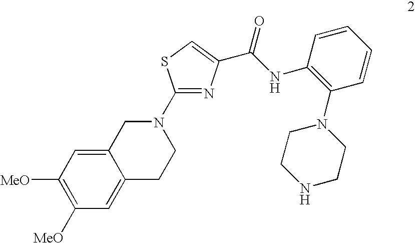

Using the method described in Example 2 and substituting 6,7-dimethoxy-1,2,3,4-tetrahydro-isoquinoline for 2,3-dihydro-1H-pyrrolo[3,4-c]pyridine, Compound 2 was prepared.

### Example 4

### Preparation of Compound 3

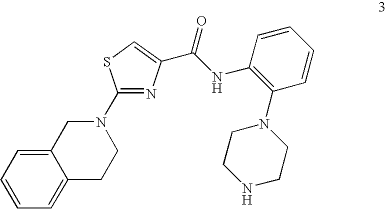

Using the method described in Example 2 and substituting 1,2,3,4-tetrahydro-isoquinoline for 2,3-dihydro-1H-pyrrolo[3,4-c]pyridine, Compound 3 was prepared.

### Example 5

### Preparation of Compound 4

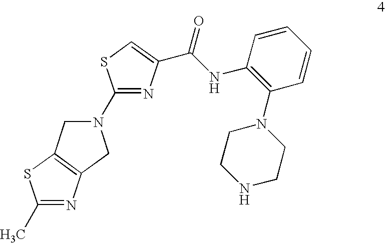

Using the method described in Example 2 and substituting 2-methyl-5,6-dihydro-4H-pyrrolo[3,4-d]thiazole for 2,3-dihydro-1H-pyrrolo[3,4-c]pyridine, Compound 4 was prepared.

### Example 6

### Preparation of Compound 5

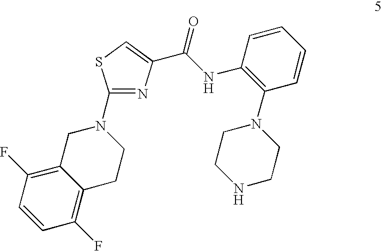

Using the method described in Example 2 and substituting 5,8-difluoro-1,2,3,4-tetrahydro-isoquinoline for 2,3-dihydro-1H-pyrrolo[3,4-c]pyridine, Compound 5 was prepared.

### Example 7

### Preparation of Compound 6

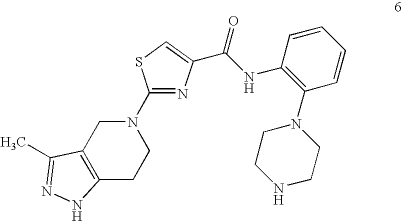

Using the method described in Example 2 and substituting 3-methyl-4,5,6,7-tetrahydro-1H-pyrazolo[4,3-c]pyridine for 2,3-dihydro-1H-pyrrolo[3,4-c]pyridine, Compound 6 was prepared.

### Example 8

### Preparation of Compound 7

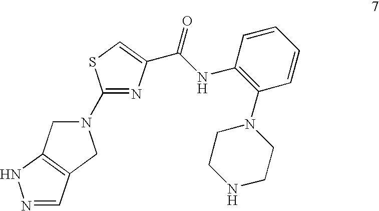

Using the method described in Example 2 and substituting 1,4,5,6-tetrahydro-pyrrolo[3,4-c]pyrazole for 2,3-dihydro-1H-pyrrolo[3,4-c]pyridine, Compound 7 was prepared.

### Example 9

### Preparation of Compound 8

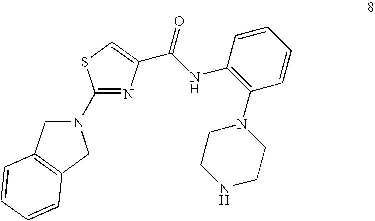

Using the method described in Example 2 and substituting 2,3-dihydro-1H-isoindole for 2,3-dihydro-1H-pyrrolo[3,4-c]pyridine, Compound 8 was prepared.

### Example 10

### Preparation of Compound 9

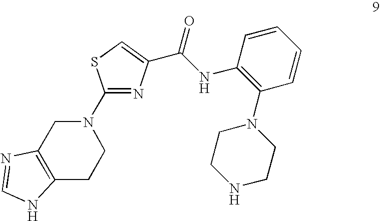

Using the method described in Example 2 and substituting 4,5,6,7-tetrahydro-1H-imidazo[4,5-c]pyridine for 2,3-dihydro-1H-pyrrolo[3,4-c]pyridine, Compound 9 was prepared.

### Example 11

### Preparation of Compound 10

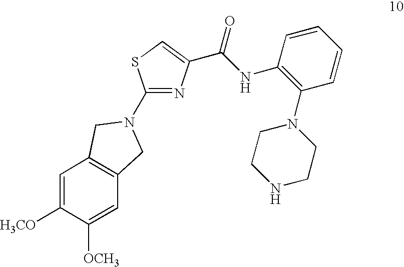

Using the method described in Example 2 and substituting 5,6-dimethoxy-2,3-dihydro-1H-isoindole for 2,3-dihydro-1H-pyrrolo[3,4-c]pyridine, Compound 10 was prepared.

### Example 12

### Preparation of Compound 11

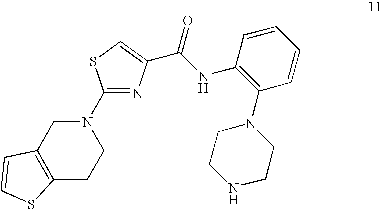

Using the method described in Example 2 and substituting 4,5,6,7-tetrahydro-1H-thieno[3,2-c]pyridine for 2,3-dihydro-1H-pyrrolo[3,4-c]pyridine, Compound 11 was prepared.

### Example 13

### Preparation of Compound 12

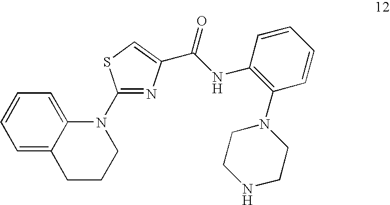

Using the method described in Example 2 and substituting 1,2,3,4-tetrahydroquinoline for 2,3-dihydro-1H-pyrrolo[3,4-c]pyridine, Compound 12 was prepared.

### Example 14

### Preparation of Compound 13

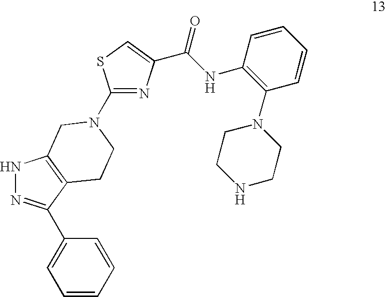

Using the method described in Example 2 and substituting 3-phenyl-4,5,6,7-tetrahydro-1H-pyrazolo[3,4-c]pyridine for 2,3-dihydro-1H-pyrrolo[3,4-c]pyridine, Compound 13 was prepared.

### Example 15

### Preparation of Compound 14

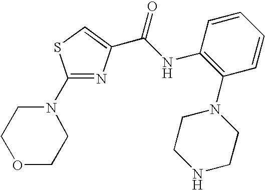

A tube containing a stir bar was charged with a solution of Compound JA (0.050 mmol, 23 mg), Pd₂(DBA)₃ (5.0 □mol, 4.6 mg), and X-Phos (0.010 mmol, 4.8 mg) in dioxane (1 mL). K₃PO₄ (0.10 mmol, 21 mg) was added to the solution and the resulting reaction was put under a nitrogen atmosphere. Morpholine (8.7 mg, 0.10 mmol) was added to the reaction mixture via a syringe under a N₂ atmosphere. The tube put into an oil bath at 100° C. and the reaction was allowed to stir at this temperature for about 15 hours, then cooled to room temperature. The reaction mixture was then diluted with acetonitrile (5 mL), the resulting solution was centrifuged for about 2 hours at a speed of about 1000 rpm, and the supernatant was collected and concentrated in vacuo. To the resulting residue was added TFA (0.5 mL) and the resulting solution was allowed to stand for 10 minutes, then concentrated in vacuo. The resulting residue was purified using reverse phase HPLC to provide Compound 14.

### Example 16

### Preparation of Compound 15

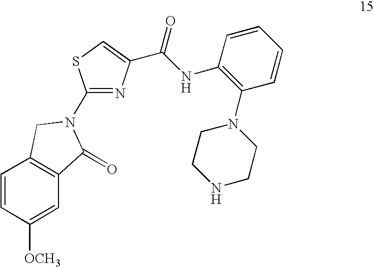

Using the method described in Example 15 and substituting 6-methoxy-2,3-dihydro-isoindol-1-one for morpholine, Compound 15 was prepared.

### Example 17

### Preparation of Compound 16

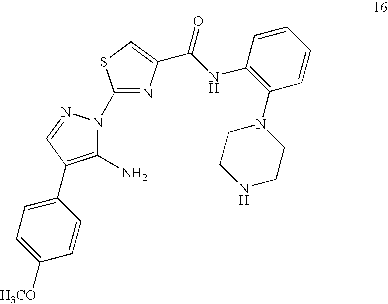

A tube containing a stir bar was charged with a solution of 4-(4-methoxy-phenyl)-2H-pyrazol-3-ylamine (0.10 mmol), Pd₂(DBA)₃ (5.0 □mol, 4.6 mg), Xantphos (0.010 mmol, 5.8 mg) and Compound JA (0.050 mmol, 23 mg) in dioxane (1 mL). To the solution was then added K₃PO₄ (0.10 mmol, 21 mg) and the reaction tube was flushed with N₂ then sealed tightly. The resulting reaction was heated to 100° C. and allowed to stir at this temperature for about 15 hours, then cooled to room temperature. The reaction mixture was then diluted with acetonitrile (5 mL), the resulting solution was centrifuged for about 2 hours at a speed of about 1000 rpm, and the supernatant was collected and concentrated in vacuo. To the resulting residue was added TFA (0.5 mL) and the resulting solution was allowed to stand for 10 minutes, then concentrated in vacuo. The resulting residue was purified using reverse phase HPLC to provide Compound 16.

### Example 18

### Preparation of Compound 17

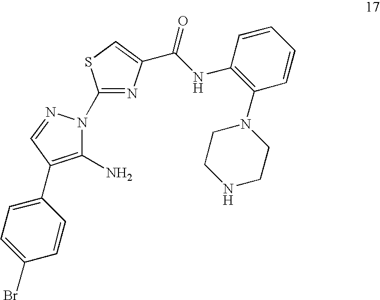

Using the method described in Example 17 and substituting 4-(4-bromo-phenyl)-2H-pyrazol-3-ylamine for 4-(4-methoxy-phenyl)-2H-pyrazol-3-ylamine, Compound 17 was prepared.

### Example 19

### Preparation of Compound 18

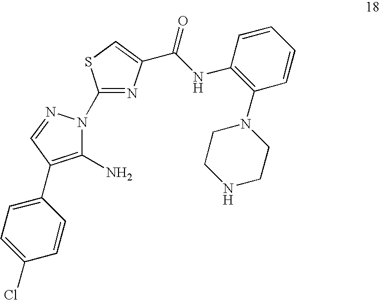

Using the method described in Example 17 and substituting 4-(4-chloro-phenyl)-2H-pyrazol-3-ylamine for 4-(4-methoxy-phenyl)-2H-pyrazol-3-ylamine, Compound 18 was prepared.

### Example 20

### Preparation of Compound 19

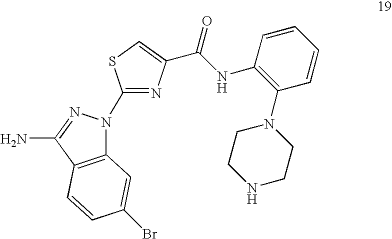

Using the method described in Example 17 and substituting 6-bromo-1H-indazol-3-1.0 ylamine for 4-(4-methoxy-phenyl)-2H-pyrazol-3-ylamine, Compound 19 was prepared.

### Example 21

### Preparation of Compound 20

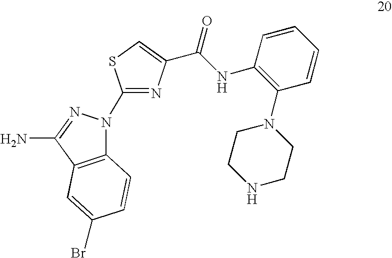

Using the method described in Example 17 and substituting 5-bromo-1H-indazol-3-ylamine for 4-(4-methoxy-phenyl)-2H-pyrazol-3-ylamine, Compound 20 was prepared.

### Example 22

### Preparation of Compound 21

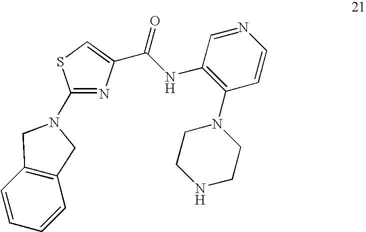

### Step 1—Synthesis of Intermediate Compound B

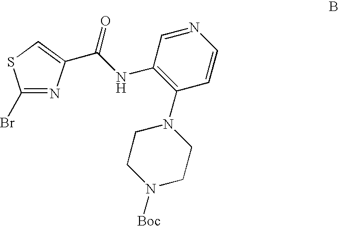

A solution of 4-chloro-3-nitro-pyridine (2.0 mmol, 0.32 g), triethylamine (3.0 mmol, 0.42 mL) and piperazine-1-carboxylic acid tert-butyl ester (2.5 mmol, 0.47 g) in dioxane (2 mL) was irradiated using microwave for 8 minutes at a temperature of 150° C. The solution was then cooled to room temperature and concentrated in vacuo and the resulting residue was purified using flash column chromatography on silica gel (eluent: ethyl acetate) to provide 4-(3-nitro-pyridin-4-yl)-piperazine-1-carboxylic acid tert-butyl ester as a yellow solid (633 mg, quantitative yield). NMR (400 MHz, CDCl₃) □ 8.87 (s, 1H), 8.40 (d, J=5.6 Hz, 1H), 6.87 (d, J=6.0 Hz, 1H), 3.68-3.56 (m, 4H), 3.32-3.18 (m, 4H), 1.48 (s, 9H).

The 4-(3-nitro-pyridin-4-yl)-piperazine-1-carboxylic acid tert-butyl ester (633 mg) was then diluted with MeOH/EtOAc (1:1, 10 mL) and to the resulting solution was added Pd on carbon (5% Pd). The resulting reaction mixture was stirred under a hydrogen atmosphere at room temperature for about 15 hours. The reaction mixture was filtered through a pad of celite and the filtrate was concentrated in vacuo to provide 4-(3-amino-pyridin-4-yl)-piperazine-1-carboxylic acid tert-butyl ester as a solid form. HPLC-MS RT=1.10 min, mass calculated for formula C₁₄H₂₂N₄O₂ 278.17, observed LCMS m/z 279.28 (M+H).

To a solution of 2-bromo-thiazole-4-carboxylic acid (0.78 mmol, 0.16 g), N,N-diisopropylethylamine (1.5 mmol, 0.26 mL) and HATU (0.78 mmol, 0.30 g) in DMF (10 mL) was added 4-(3-amino-pyridin-4-yl)-piperazine-1-carboxylic acid tert-butyl ester (0.78 mmol, 0.22 g). The reaction mixture was heated to 80° C. and allowed to stir at this temperature for about 15 hours, after which time the reaction mixture was cooled to room temperature and concentrated in vacuo. The resulting crude residue was purified using flash column chromatography on silica gel (eluent: ethyl acetate) to provide Compound B as a yellow solid. HPLC-MS RT=1.40 min, mass calculated for formula C₁₈H₂₂BrN₅O₃S 467.06, observed LCMS m/z 468.05 (M+H).

### Step 2—Synthesis of Compound 21

Using the method described in Example 2 and substituting Compound B for Compound A, and 2,3-dihydro-1H-isoindole for 2,3-dihydro-1H-pyrrolo[3,4-c]pyridine, Compound 21 was prepared.

### Example 23

### Preparation of Compound 22

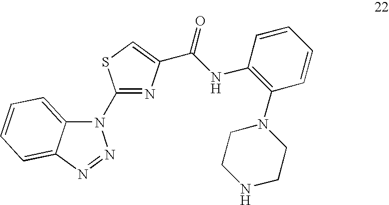

### Step 1—Synthesis of Compound C

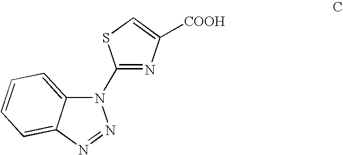

Benzotriazole (1.20 mmol, 143 mg), K₃PO₄ (1.5 mmol, 0.32 g), Pd₂(DBA)₃ (40.0 □mol, 36.6 mg), X-Phos (0.12 mmol, 57 mg) and 2-bromo-thiazole-5-carboxylic acid ethyl ester (1.00 mmol, 236 mg) were loaded into a Schlenk tube containing a stir bar. The Schlenk tube was capped with a rubber septum, evacuated and put under a nitrogen atmosphere. Toluene (2 mL) was added through the septum via a syringe, then the tube was sealed with a Teflon screw cap under a flow of nitrogen, and put into an oil bath at 100° C. The reaction was heated to 100° C. and allowed to stir at this temperature for about 15 hours, after which time, the reaction mixture was cooled to room temperature and filtered through a pad of celite. The filtrate was concentrated in vacuo and the resulting residue was purified using flash column chromatography on silica gel (eluent: Hexane/EtOAc (6:1)) to provide 2-benzotriazol-1-yl-thiazole-4-carboxylic acid ethyl ester as a white solid. ¹H NMR (400 MHz, CDCl₃) □ 8.57 (d, J=8.4 Hz, 1H), 8.14 (d, J=8.0 Hz, 1H), 8.06 (s, 1H), 7.73-7.68 (m, 1H), 7.54-7.49 (m, 1H), 4.46 (q, J=7.2 Hz, 2H), 1.45 (t, J=7.2 Hz, 3H).

2-benzotriazol-1-yl-thiazole-4-carboxylic acid ethyl ester was diluted with concentrated aqueous hydrochloric acid and the resulting solution was heated to reflux and allowed to stir at this temperature for about 15 hours. The reaction mixture was then cooled to room temperature and lyophilized to provide Compound C as an ammonium chloride salt.

### Step 2—Synthesis of Compound 22

To a solution of 2-benzotriazol-1-yl-thiazole-4-carboxylic acid (0.050 mmol, 14 mg), N,N-diisopropylethylamine (0.25 mmol, 44 □L) and HATU (0.050 mmol, 19 mg) in DMF (0.5 mL) was added 4-(2-aminophenyl)-piperazine-1-carboxylic acid tert-butyl ester (0.10 mmol, 28 mg). The reaction mixture was heated to 80° C. and allowed to stir at this temperature for about 15 hours, after which time the reaction mixture was cooled to room temperature and concentrated in vacuo. To the resulting solid residue was added TFA (0.5 mL) and the resulting solution was allowed to stand for 10 minutes, then was concentrated in vacuo. The resulting residue was purified using reverse phase HPLC to provide Compound 22.

### Example 24

### Preparation of Compound 23

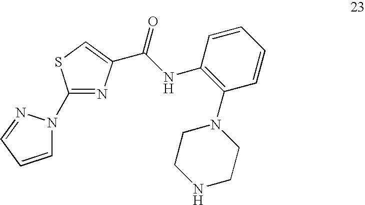

In 20 mL vial containing a stir bar (vial 1) is charged with a solution of 4-{2-[(2-bromo-thiazole-4-carbonyl)-amino]-phenyl}-piperazine-1-carboxylic acid tert-butyl ester acid (107 μmol, 50 mg) and 1,4-dioxane (1 mL). A second 20 mL vial containing a stir bar (vial 2) is charged with a solution of pyrazole (4 eq, 428 μmol, 29.1 mg) and 1,−4 dioxane (2 mL). To the solution in vial 2 is added NaH (60% dispersion in mineral oil, 4 eq, 428 μmmol, 17.2 mg). The resulting reaction is allowed to stir for 15 minutes, then is added to the solution in vial 1. Vial 1 is sealed and the resulting reaction inside vial 1 is heated to 100° C. and allowed to stir at this temperature for about 18 hours. LC/MS analysis confirmed disappearance of the starting material and the reaction mixture was concentrated in vacuo. The resulting crude residue was diluted with dichloromethane (2 mL), filtered through celite and the filtrate was purified using flash column chromatography on silica gel (eluent: gradient from 100% hexanes to 60% ethyl acetate in hexanes) provided an intermediate white solid product. ¹H NMR (400 MHz, CD₃CN) [symbol] 10.35-10.25 (br s, 1H), 8.49-8.46 (dd, J=8, 1.6 Hz, 1H), 8.39-8.37 (d, J=2.8 Hz, 1H), 8.04 (s, 1H), 7.84-7.83 (d, J=1.6 Hz, 1H), 7.30-7.27 (dd, J=8, 1.6 Hz, 1H), 7.25-7.20 (td, J=8, 1.6 Hz, 1H), 7.18-7.13 (td, J=8, 1.6 Hz, 1H), 3.70-3.63 (br t, J=4.8 Hz, 4H), 2.91-2.86 (m, J=4.8 Hz, 4H), 1.48 (s, 9H). The intermediate white solid product was diluted with a 9:1 solution of TFA:H₂O (2 mL). The resultant solution was shaken for 2 hours at room temperature and the reaction mixture was concentrated in vacuo. The resulting residue was purified using reverse-phase HPLC and lyophilized with aqueous HCl(1 M) to provide Compound 23 as a dihydrochloride salt (15.43 mg).

The following illustrative compounds of the invention were prepared using this method with appropriate reactants:

| | | LCMS | |
|---|---|---|---|
| | | MH⁺ | HPLC |
| No. | Structure | m/z | MS t_{R} |
| 118 | 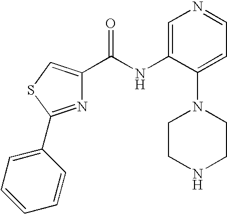 | 366.21 | 1.74 |
| 204 | 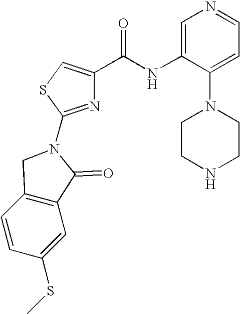 | 467.23 | 2.13 |
| 242 | 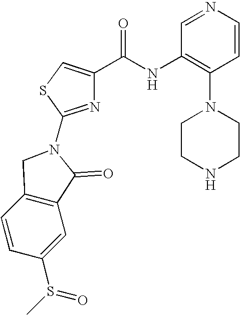 | 483.11 | 1.48 |
| 243 | 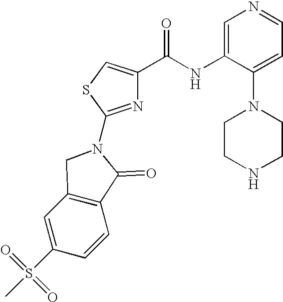 | 499.08 | 1.26 |
| 267 | 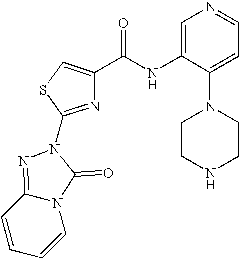 | 445.69 (M + Na) | 0.749 |
| 269 | 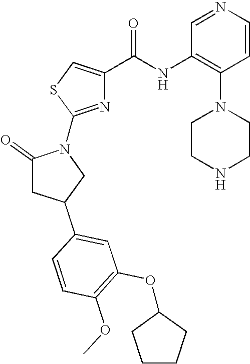 | 563.26 | 2.71 |
| 293 | 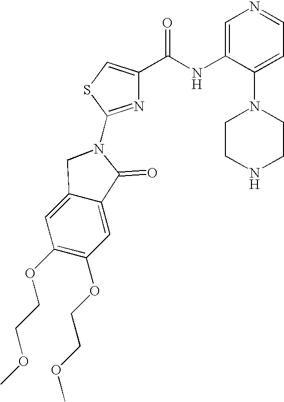 | 569.37 | 2.07 |
| 333 | 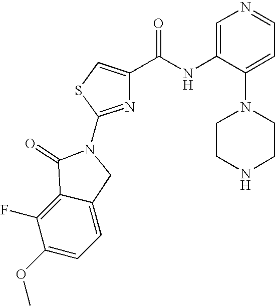 | 469.22 | 1.92 |

### EXAMPLE

### Preparation of Compound 24

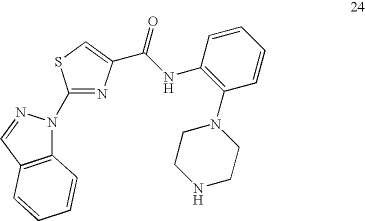

Using the method described in Example 24 and substituting indazole for pyrazole, Compound 24 was prepared as a dihydrochloride salt.

### Example 26

### Preparation of Compound 25

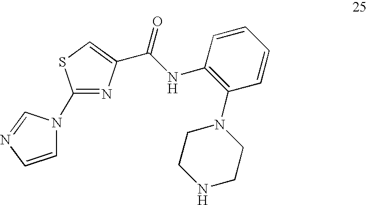

Using the method described in Example 24 and substituting imidazole for pyrazole, Compound 25 was prepared as a dihydrochloride salt.

### Example 27

### Preparation of Compound 26

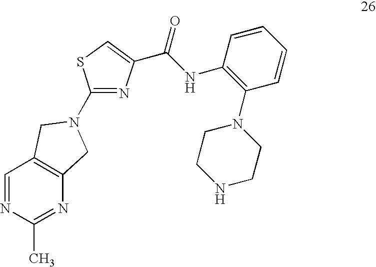

Using the method described in Example 24 and substituting 2-methyl-6,7-dihydro-5H-pyrrolo[3,4-d]pyrimidine dihydrochloride for pyrazole, Compound 26 was prepared as a dihydrochloride salt.

### Example 28

### Preparation of Compound 27

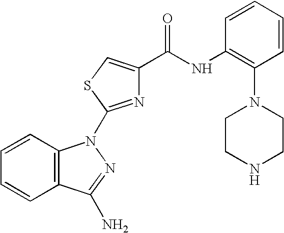

A 20 mL vial containing a stir bar was charged with 4-{2-[(2-bromo-thiazole-4-carbonyl)-amino]-phenyl}-piperazine-1-carboxylic acid tert-butyl ester (107 μmol, 50 mg), Pd₂(DBM)₃ (0.05 eq, 5.4 μmol, 4.9 mg), Xant-Phos (0.1 eq, 10.7 μmol, 6.2 mg), K₃PO₄ (2 eq, 214 μmol, 45.5 mg), 3-aminoindazole (2 eq, 214 μmol, 28.5 mg) and toluene (3 mL). The vial was flushed with argon, capped and sealed and then put in an oil bath at 140° C. The reaction was then allowed to stir at this temperature for about 18 hours. LC/MS confirms the presence of 2 products. The reaction mixture was concentrated in vacuo and the resulting residue was diluted with dichloromethane (2 mL) and filtered through celite. The filtrate was then purified using reverse-phase HPLC and the 2 separated products were characterized using LC/MS (the first product had a retention time=5.76 min, and m+1=520.24; the second product has a retention time=5.99 minutes, and m+1=520.35). The second product was diluted with a 9:1 mixture of TFA:H₂O (2 mL) and the resulting solution was shaken for 2 hours at room temperature. The reaction mixture was concentrated in vacuo and the resulting residue was purified using reverse-phase HPLC and lyophilized with aqueous HCl (1M) to provide Compound 27 as a dihydrochloride salt.

### Example 29

### Preparation of Compound 28

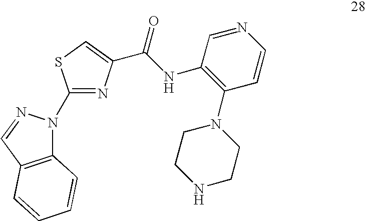

Using the method described in Example 24 and substituting 4-{3-[(2-Bromo-thiazole-4-carbonyl)-amino]-pyridin-4-yl}-1-Boc-piperazine for 4-{2-[(2-bromo-thiazole-4-carbonyl)-amino]-phenyl}-piperazine-1-carboxylic acid tert-butyl ester acid and indazole for pyrazole, Compound 28 was prepared as a dihydrochloride salt.

### Example 30

### Preparation of Compound 29

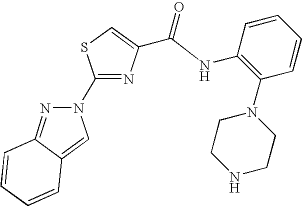

A 20 mL vial containing a stir bar (vial 1) was charged with a solution of 4-{2-[(2-bromo-thiazole-4-carbonyl)-amino]-phenyl}-piperazine-1-carboxylic acid tert-butyl ester acid (321 μmol, 150 mg). To this is added 2 mL 1,4-dioxane. A second 20 mL vial containing a stir bar (vial 2) was charged with a solution of indazole (3 eq, 963 μmol, 114 mg) and 4 mL 1,4-dioxane. To the solution in vial 2 was then added NaH (60% dispersion in mineral oil, 3 eq, 963 μmol, 38.5 mg). The resulting reaction was allowed to stir at room temperature for about 15 minutes, then the reaction mixture was added to the solution in vial 1. Vial 1 was then sealed, placed in an oil bath at 100° C. and the reaction mixture was allowed to stir at this temperature for about 5 hours. The reaction mixture was then concentrated in vacuo and the resulting residue was diluted with dichloromethane (2 mL) and filtered through celite. The resulting residue was purified using flash column chromatography on silica gel (eluent: gradient from 100% hexanes to 70% ethyl acetate in hexanes) provided a product which was collected and diluted with a 9:1 mixture of TFA:H₂O (3 mL) and the resulting solution was allowed to stir for 2 hours at room temperature. concentrated in vacuo and the resulting residue was purified by reverse-phase HPLC and shown to contain two products. The first product has a retention time of 3.73 minutes with a visible mass of m+1=405.23. This product was lyophilized with aqueous HCl to provide Compound 29 as a dihydrochloride salt (12.45 mg).

### Example 31

### Preparation of Compound 30

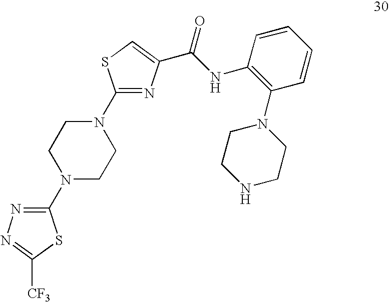

A 2 mL microwave vial was charged with a solution of 4-{2-[(2-bromo-thiazole-4-carbonyl)-amino]-phenyl}-piperazine-1-carboxylic acid tert-butyl ester (107 mmol, 50 mg) in acetonitrile (2 mL). To this solution was added a solution of 1-(5-Trifluoromethyl-[1,3,4]thiadiazol-2-yl)-piperazine (160 mmol, 38 mg) in DIEA (160 μmol, 28 μL) and the resulting reaction was microwaved at 180° C. for about 15 minutes. The reaction mixture was then concentrated in vacuo and the resulting residue was diluted with a 9:1 mixture of TFA:H₂O (2 mL) and the resulting solution was shaken for about 2 hours at room temperature. The reaction mixture was then concentrated in vacuo and the resulting residue was purified using reverse-phase HPLC and lyophilized with aqueous HCl to provide Compound 30 as a dihydrochloride salt (53.34 mg).

LCMS data and HPLC retention times for Illustrative Anilinopiperazine Derivatives are provided in the table below, wherein the compound numbers in Table 1 correspond to the compound numbering in the specification.

| | Observed LCMS | HPLC-MS |
|---|---|---|
| Compound | m/z (M + H) | retention time (min) |
| 1 | 407.28 | 2.56 |
| 2 | 480.33 | 4.06 |
| 3 | 420.40 | 4.24 |
| 4 | 427.23 | 3.72 |
| 5 | 456.29 | 4.37 |
| 6 | 424.28 | 2.93 |
| 7 | 396.29 | 3.27 |
| 8 | 406.26 | 4.13 |
| 9 | 410.29 | 2.36 |
| 10 | 466.26 | 3.91 |
| 11 | 426.26 | 4.11 |
| 12 | 420.27 | 4.21 |
| 13 | 486.22 | 3.82 |
| 14 | 374.25 | 3.28 |
| 15 | 450.12 | 3.72 |
| 16 | 476.32 | 3.75 |
| 17 | 524.26 | 4.11 |
| 18 | 480.32 | 4.05 |
| 19 | 498.22 | 3.84 |
| 20 | 498.25 | 3.81 |
| 21 | 407.28 | 2.70 |
| 22 | 406.21 | 3.48 |
| 23 | 355.33 | 3.20 |
| 24 | 405.28 | 3.89 |
| 25 | 355.27 | 2.31 |
| 26 | 422.27 | 3.28 |
| 27 | 420.10 | 3.45 |
| 28 | 406.12 | 2.31 |
| 29 | 405.23 | 3.73 |
| 30 | 525.21 | 3.85 |
| 31 | 487.5 | NA |

NA = not available

### Example 32

### Preparation of Compound 45

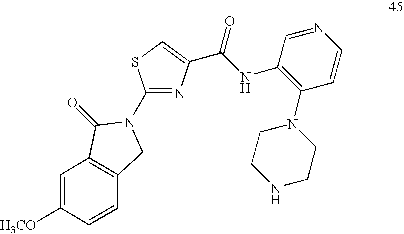

Using the method described in Example 24 and substituting 4-{3-[(2-Bromo-thiazole-4-carbonyl)-amino]-pyridin-4-yl}-1-Boc-piperazine for 4-{2-[(2-bromo-thiazole-4-carbonyl)-amino]-phenyl}-piperazine-1-carboxylic acid tert-butyl ester acid (compound B) and 6-methoxy-2,3-dihydro-isoindol-1-one, Compound 45 was prepared as a dihydrochloride salt.

### Example 33

### Preparation of Compound 43

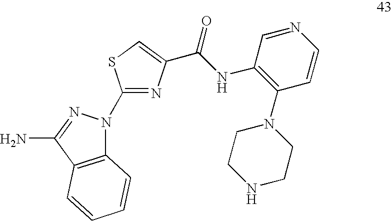

Using the method described in Example 24 and substituting 4-{3-[(2-Bromo-thiazole-4-carbonyl)amino]-pyridin-4-yl}-1-Boc-piperazine for 4-{2-[(2-bromo-thiazole-4-carbonyl)-amino]-phenyl}-piperazine-1-carboxylic acid tert-butyl ester acid and 3-amino indazole, Compound 43 was prepared as a trihydrochloride salt.

Using the same procedure and the appropriate reactants, the following compounds were prepared.

| | | LCMS | |
|---|---|---|---|
| | | MH⁺ | HPLC |
| No. | Structure | m/z | MS t_{R} |
| 84 | 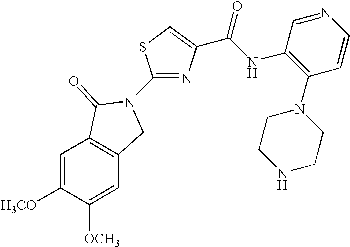 | 481.1 | 1.04 |
| 96 | 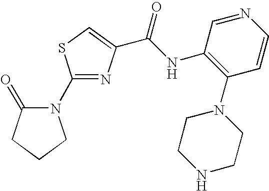 | 373.1 | 0.60 |
| 99 | 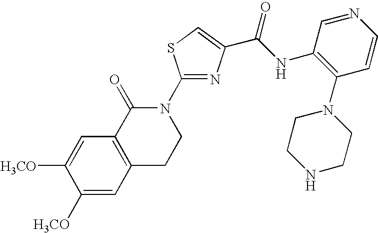 | 495.1 | 0.92 |
| 100 | 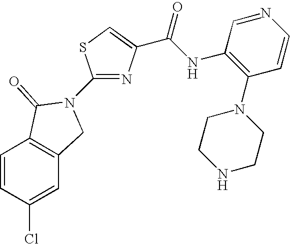 | 455.0 | 0.98 |
| 106 | 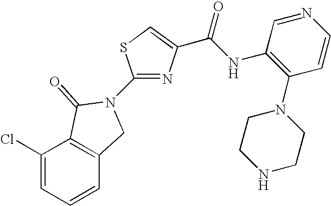 | 455.0 | 0.97 |
| 107 | 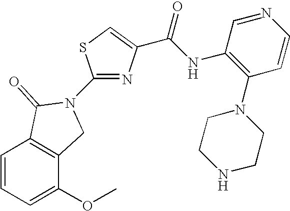 | 451.1 | 1.06 |
| 108 | 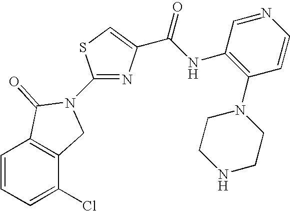 | 455.0 | 0.95 |
| 109 | 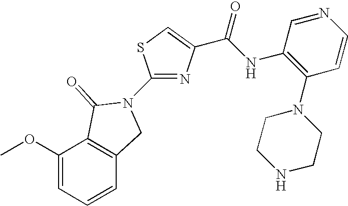 | 451.1 | 0.88 |
| 115 | 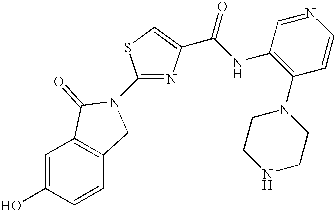 | 437.1 | 0.74 |
| 116 | 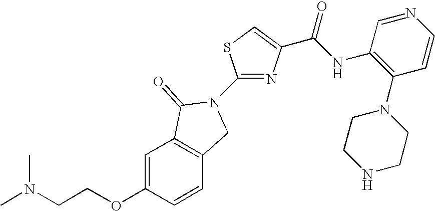 | 508.1 | 0.65 |
| 119 | 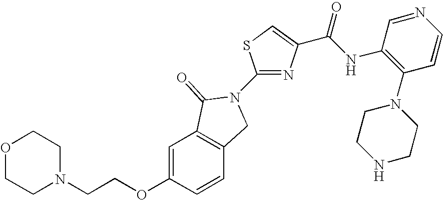 | 550.2 | 0.66 |
| 121 | 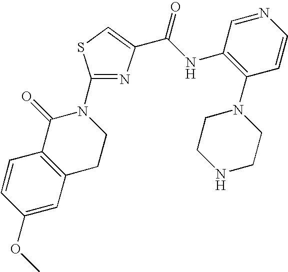 | 465.1 | 1.00 |
| 86 | 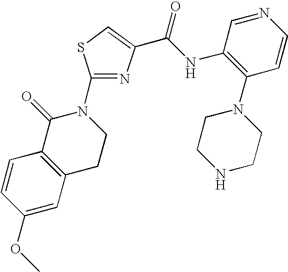 | 465.1 | 1.00 |
| 138 | 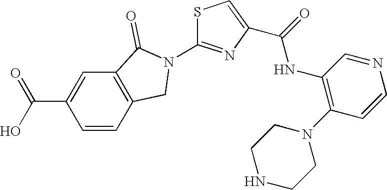 | 465.0 | 0.72 |
| 139 | 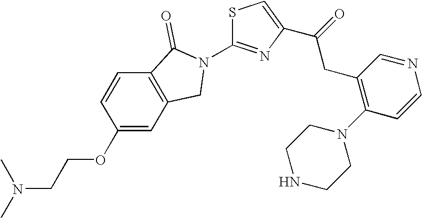 | 508.1 | 0.69 |
| 147 | 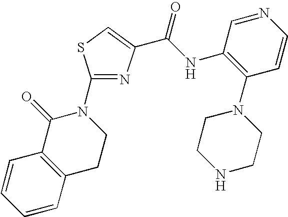 | 435.1 | 0.98 |
| 149 | 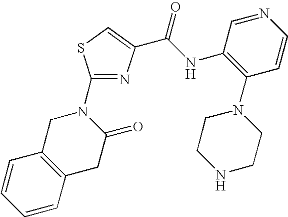 | 435.1 | 0.98 |
| 150 | 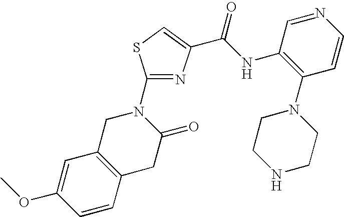 | 465.1 | 1.00 |
| 164 | 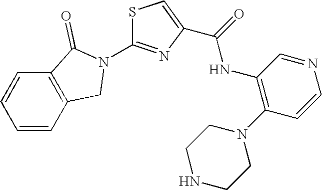 | 421.1 | 0.88 |
| 165 | 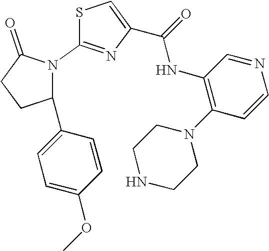 | 479.1 | 0.93 |
| 169 | 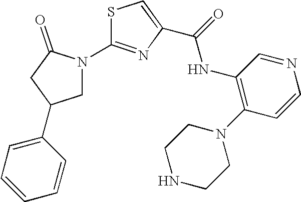 | 449.2 | 0.97 |
| 186 | 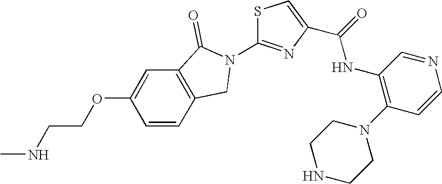 | 494.1 | 0.63 |
| 187 | 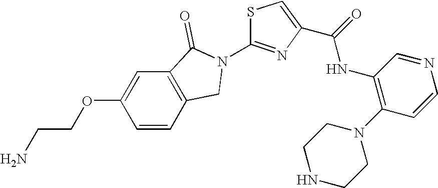 | 480.0 | 0.62 |
| 188 | 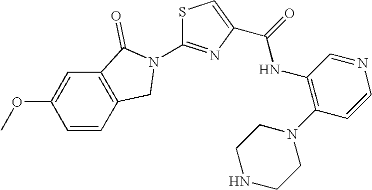 | 452.1 | 0.88 |
| 190 | 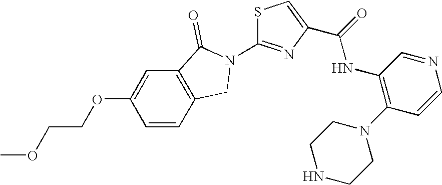 | 495.1 | 0.92 |
| 191 | 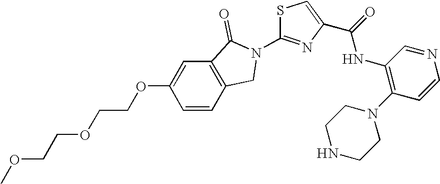 | 539.2 | 0.93 |
| 192 | 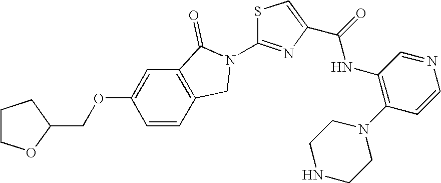 | 521.2 | 1.00 |
| 196 | 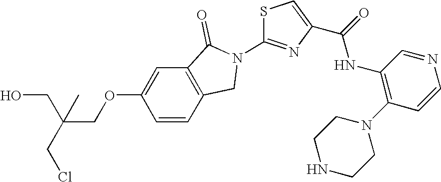 | 557.2 | 0.97 |
| 197 | 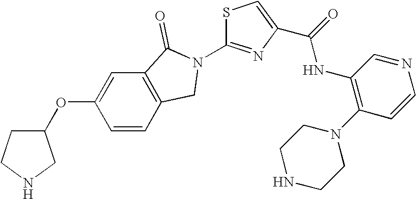 | 506.2 | 0.65 |
| 198 | 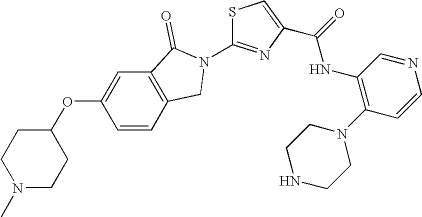 | 534.2 | 0.86 |
| 199 | 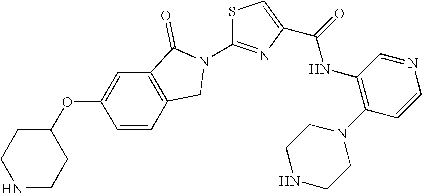 | 520.2 | 0.68 |
| 200 | 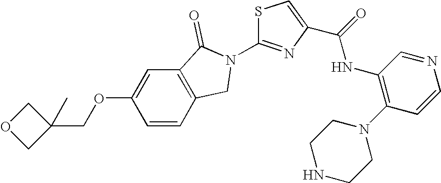 | 521.2 | 1.03 |
| 205 | 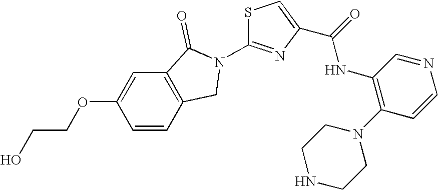 | 481.1 | 0.93 |
| 234 | 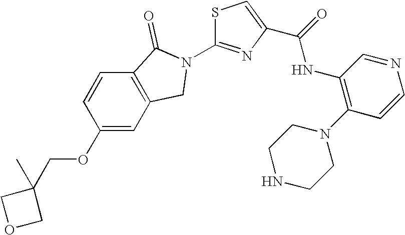 | 521.2 | 1.01 |
| 235 | 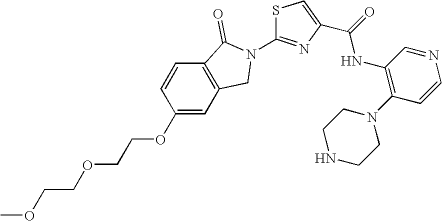 | 539.2 | 0.94 |
| 236 | 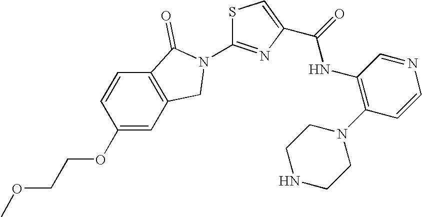 | 495.1 | 0.92 |
| 237 | 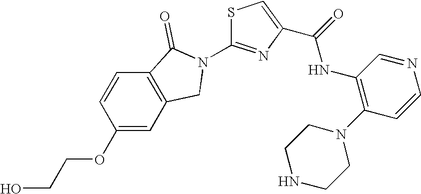 | 481.1 | 0.90 |
| 238 | 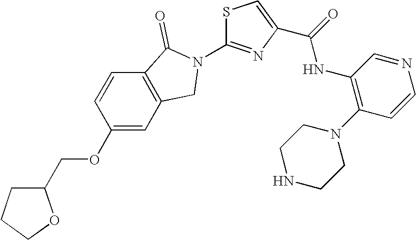 | 521.2 | 1.01 |
| 263 | 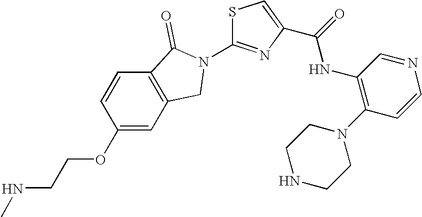 | 494.1 | 0.64 |
| 287 | 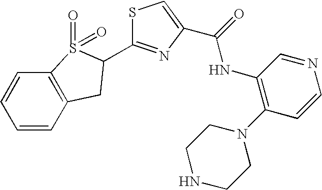 | 457.1 | 0.81 |

### Example 34

### Preparation of Intermediate Compound 34C

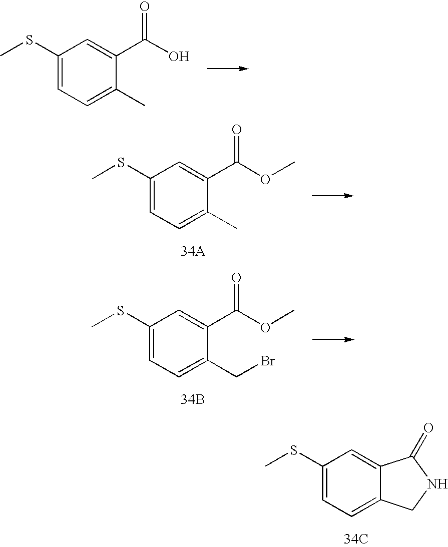

Into a solution of 2-Methyl-5-methylsulfanyl-benzoic acid (250 mg, 1.37 mmol) in 12 mL (1:1 benzene/methanol) mixture was added 2.74 mmol of (Trimethylsilyl)diazomethane. Reaction was stirred for 1.5 hours. Solvent was removed to yield 34A (2-Methyl-5-methylsulfanyl-benzoic acid methyl ester) as yellow oil which was used as is in subsequent step.

Into solution of 34A (1.034 g, 5.27 mmol) in 15 mL of carbon tetrachloride was added N-Bromosuccinimide (0.685 g, 3.85 mmol) and benzoyl peroxide (46.63 mg, 0.19 mmol). The reaction mixture was refluxed at 80° C. for 6 hours. Mixture was cooled and precipitate removed via filtration. Organic layer collected was concentrated under vacuo. Resulting crude compound 34B was dissolved in 7N NH₃ in methanol (20 mL) and heated to 85° C. in a sealed vessel for about 15 hours. Solvent was removed and crude was purified on flash silica column using ethyl acetate/hexane solvent system to provide 158 mg of compound 34A as a white powder. NMR(H¹) □ 2.51(3H), 4.30 (2H), 7.45-7.47 (m, 3H).

### Example 35

### Preparation of Intermediate Compound 35A

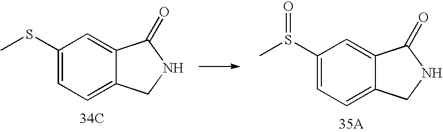

To solution of compound 34C (40 mg, 0.223 mmol) in 5 mL dichloromethane was added 3-chloroperoxybenzoic acid (55 mg, 0.223 mmol) and the reaction was stirred at room temperature for 3 hours. The reaction mixture was then cooled in an ice bath and the precipitated formed was removed via filtration. The filtrated was washed with water, dried over anhydrous sodium sulfate, filtered and concentrated in vacuo to provide compound 35A, which was used without further purification. NMR of crude indicate a shift of S—CH3 peak from 2.51 to 2.84 ppm

### Example 36

### Preparation of Intermediate Compound 36A

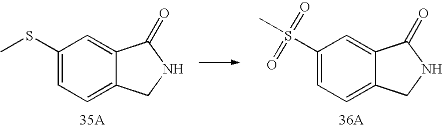

Using the method described in Example 35, compound 35A was converted to compound 36A.

### Example 37

### Preparation of Intermediate Compound 37A

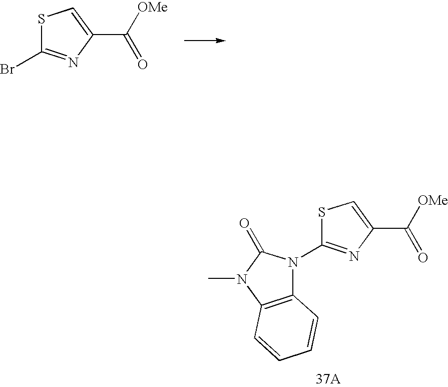

To a solution of 2-bromo-4-carbomethoxythiazole (1.5 g, 6.78 mmol) in dioxane (80 mL) at room temperature was added 1-methyl-2-benzimidazolone (1.0 g, 6.78 mmol) followed by CuI (0.13 g, 0.68 mmol), K₂CO₃ (1.0 g, 7.47 mmol), trans-N,N-dimethylcyclohexane (0.21 mL, 1.35 mmol). The mixture was degassed under house vacuum and filled with N₂ six times and heated to 90° C. The mixture was stirred for 12 hours, cooled to rt, and concentrated under reduced pressure. The crude product was purified using flash chromatography using a 20:1 mixture of CH₂Cl₂/MeOH to provide 1.8 g (92% yield) of the title compound as an off-white solid. LC-MS [M+H]=290.2; 98% purity.

### Example 38

### Preparation of Intermediate Compound 38A

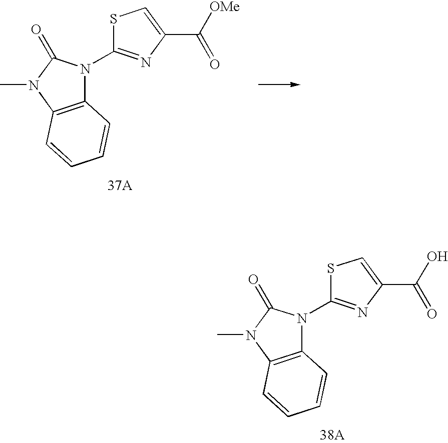

To a solution of compound 37A (0.18 g, 0.59 mmol) in THF (1.5 mL) at 0° C. was added dropwise a 1M solution of LiOH (1.18 mL). The resulting reaction was allowed to warm to room temperature and allowed to stir for 12 hours. The mixture was concentrated under reduced pressure and taken up in H₂O (2 mL). The mixture was treated with concentrated HCl until pH=4 was attained. The mixture was concentrated under reduced pressure to provide compound 38A (0.15 g, 92% yield) as an orange solid which was used without further purification. LC-MS [M+H]=276.2; 96% purity

### Example 39

### Preparation of Intermediate Compound 39A

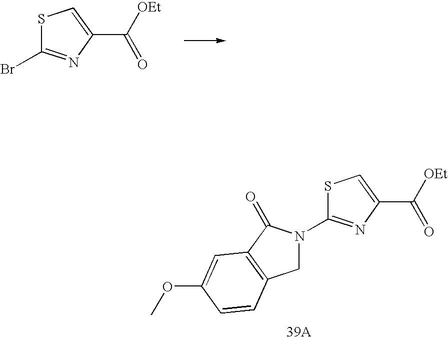

To a pressure tube charged with 2-bromo-4-carboethoxythiazole (2.5 g, 10.6 mmol) and a stir bar was added 6-methoxyisoindolin-1-one (2.1 g, 12.7 mmol), K₃PO₄ (4.9 g, 23.3 mmol), Pd₂(dba)₃ (0.58 g, 0.64 mmol), Xant-Phos (0.62 g, 1.1 mmol). Dioxane (20 mL) was added and N₂ was bubbled thru the solution for 10 min before the vessel was capped. The mixture stirred at 105° C. for 12 h and was cooled to rt. The mixture was filtered thru a pad of Celite and was washed with CH₂Cl₂/MeOH (20:1; 2×10 mL). The resulting filtrate was concentrated under reduced pressure and place under high vacuum. The crude product was purified using flash chromatography using a gradient from CH₂Cl₂ to 97:3 CH₂Cl₂/acetone to provide 3.1 g (91% yield) of compound 39A as a brown solid. LC-MS [M+H]=400.2; 98% purity.

### Example 40

### Preparation of Intermediate Compound 40A

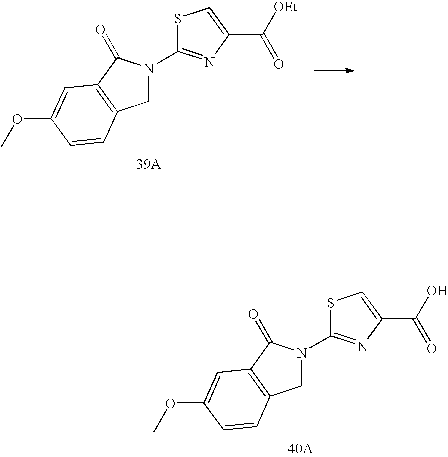

To a solution of the compound 39A (0.67 g, 2.1 mmol) in THF/MeOH/H₂O (2:2:1; 12.5 mL total) at room temperature was added LiOH.H₂O (97 mg, 2.3 mmol) in one portion. The resulting solution was stirred at 40° C. for 12, cooled to rt, and concentrated under reduced pressure. The crude material and taken up in H₂O (20 mL) and was treated with concentrated HCl until pH 3 was attained. The mixture was concentrated under reduced pressure to provide 0.58 g (95% yield) of compound 40A a pale white solid which was used without further purification. MS [M+H]=290.9

### Example 41

### Preparation of Intermediate Compound 41A

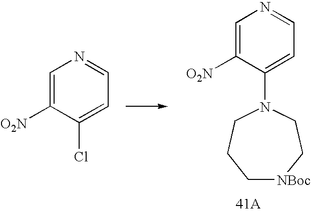

To a solution of 4-chloro-3-nitropyridine (2.0 g, 12.5 mmol) in dioxane (25 mL) was added DIPEA (3.2 mL, 18.7 mmol) followed by Boc-homopiperazine (3.0 g, 15.0 mmol). The resulting mixture was stirred at 110° C. for 12 hours, cooled to rt, and concentrated to dryness.

The mixture was partitioned between sat. aq NaHCO₃ (4 mL) and CH₂Cl₂ (15 mL) and the layers were separated. The aqueous layer was extracted with CH₂Cl₂ (2×15 mL) and the organic layers were combined. The organic layer was washed with brine (1×4 mL), dried (Na₂SO₄), filtered, and concentrated under reduced pressure. The crude product was purified using flash chromatography using a 50:1 mixture of CH₂Cl₂/MeOH as eluent to provide 3.7 g (93% yield) of compound 41A as a yellow solid. LC-MS [M+H]=323.2; 98% purity.

### Example 42

### Preparation of Intermediate Compounds 42A-42D

Intermediate compounds 42A-42D, shown in the table below, were prepared by reacting the indicated chloro derivatives with the indicated amines according to the method described in Example 41.

| | | | 1. Yield |
|---|---|---|---|
| | | | (%) |
| | | | 2. LC-MS |
| Chloro | | | (M + |
| derivative | Amine | Product | H) |
| 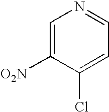 | 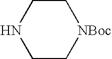 | 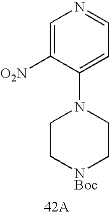 | 1. 98 2. 309.2 |
| 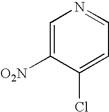 | 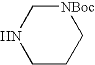 | 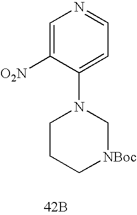 | 1. 80 2. 309.2 |
| 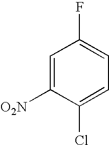 | 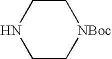 | 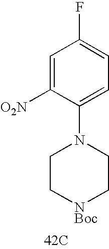 | 1. 94 2. 326.1 |
| 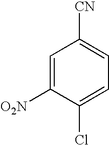 | 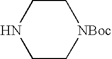 | 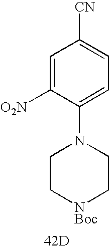 | 1 .98 2. 333.2 |

### Example 43

### Preparation of Intermediate Compound 43A

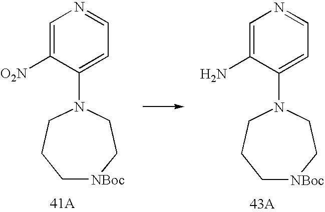

To a mixture of compound 41A (3.5 g, 11.1 mmol) in MeOH/EtOAc (1:1; 100 mL) at room temperature was added 5% Pd/C (1.2 g). The resulting mixture was degassed and filled with N₂ and finally with H₂ (balloon). The mixture was stirred for 12 h at room temperature and was purged to N₂. The reaction mixture was filtered through a pad of Celite which was washed with the MeOH/EtOAc (1:1; 3×25 mL). The resultant filtrate was concentrated under reduced pressure and placed under high vacuum to provide 3.2 g (99% yield) of compound 43A as a yellow semisolid. LC-MS [M+H]=293.2; 87% purity. This material was used without further purification.

### Example 44

### Preparation of Intermediate Compounds 44A-44C

Following the method described in Example 43, the indicated nitro derivatives were converted to the corresponding amino derivatives 44A-44C.

| | | 1. Yield (%) |
|---|---|---|
| Nitro derivative | Product | 2. LC-MS (M + H) |
| 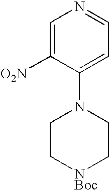 | 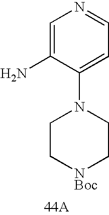 | 1. 98 2. 309.2 |
| 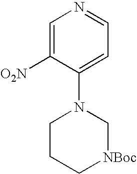 | 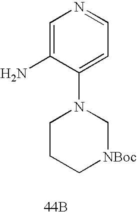 | 1. 80 2. 309.2 |
| 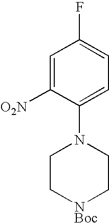 | 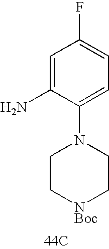 | 1. 94 2. 326.1 |

### Example 45

### Preparation of Intermediate Compound 45A

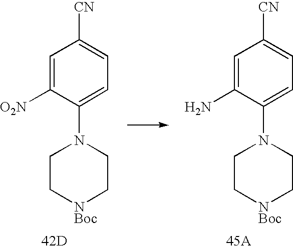

To a mixture of compound 42D (0.5 g, 1.5 mmol) in THF (15 mL) at room temperature was added ammonium formate (0.95 g, 15.1 mmol) followed by 10% Pd/C (50 mg). The resulting mixture heated to 65° C., stirred for 30 min, and was cooled to rt. The reaction mixture was filtered through a pad of Celite which was washed with the EtOH (2×5 mL) and CH₂Cl₂ (2×5 mL). The resultant filtrate was concentrated under reduced pressure and placed under high vacuum to provide 0.46 g (99% yield) of compound 45A as a yellow semisolid. LC-MS [M+H]=303.2; 99% purity. This material was used without further purification.

### Example 46

### Preparation of Intermediate Compound 46A

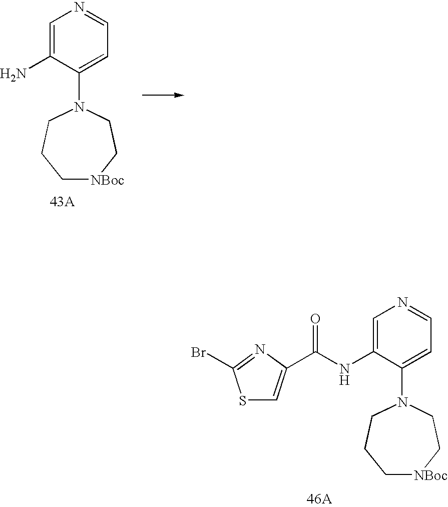

To a solution of compound 43A (1.1 g, 5.2 mmol) in DMF (10 mL) was added N,N-diisopropylethylamine (2.7 ml, 15.4 mmol) and HATU (2.2 g, 5.6 mmol) in DMF (10 mL) was added aniline (1.5 g, 5.2 mmol) from Preparative Example 10. The reaction mixture was stirred at room temperature for 72 h and then concentrated in vacuo. The crude residue was taken up in EtOAc (50 mL) and sat. aq NaHCO₃ (2 mL) was added. The layers were separated and the organic layer was washed with sat. aq. NaHCO₃ (1×2 mL) and brine (1×2 mL). The organic layer was dried (Na₂SO₄), filtered, and concentrated under reduced pressure. The crude product was purified using preparative thin-layer chromatography using a 40:1 mixture of CH₂Cl₂/MeOH as eluent to provide 1.9 g (75% yield) of compound 46A as a light yellow solid as the title compound. LC-MS [M+H]=483.2; 89% purity.

### Example 47

### Preparation of Intermediate Compounds 47A-47C

Following the method described in Example 46, utilizing 2-bromo-thiazole-4-carboxylic acid and the indicated amines, intermediate compounds 47A-47C were made.

| | | 1. Yield (%) |
|---|---|---|
| Amine | Product | 2. LC-MS (M + H) |
| 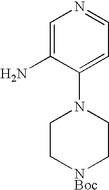 | 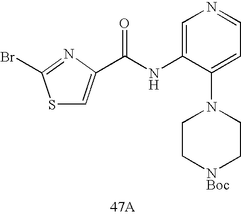 | 1. 63 2. 470.3 |
| 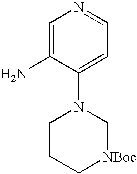 | 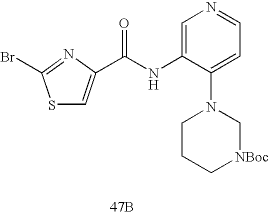 | 1. 35 2. 470.3 |
| 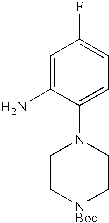 | 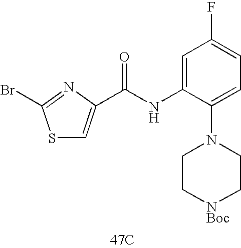 | 1. 67 2. 470.3 |

### Example 48

### Preparation of Intermediate Compound 48A

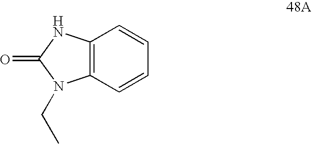

Compound 48A was prepared using the method described in US Patent Publication No. 2007/0072928.

### Example 49

### Preparation of Intermediate Compound 49A

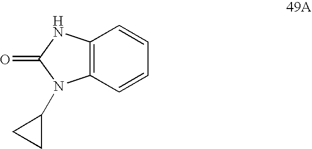

Compound 49A was prepared using the method described in US Patent Publication No. 2007/0072928.

### Example 50

### Preparation of Intermediate Compound 50A

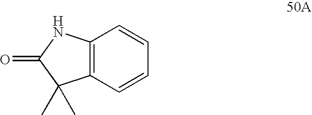

Compound 50A was prepared using the method described in J. Med. Chem. 1986, 29, 1832.

### Example 51

### Preparation of Intermediate Compound 51A

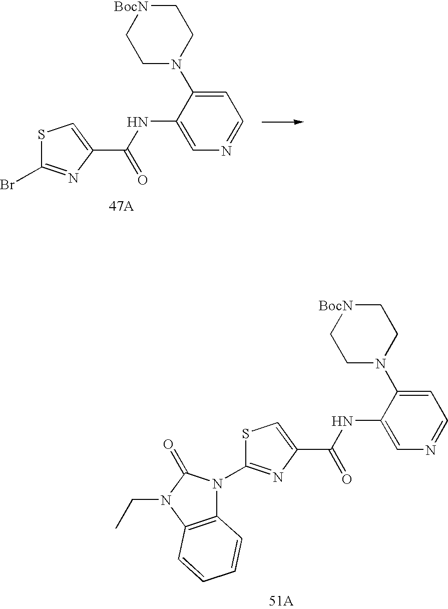

To a solution of the compound 47A (0.35 g, 0.75 mmol) was added compound 48A (0.12 g, 0.75 mmol), CuI (14 mg, 0.075 mmol), K₂CO₃ (114 mg, 0.83 mmol), and trans-N,N-dimethylcyclohexane (23 □L, 0.15 mmol). The mixture was degassed under house vacuum and filled with N₂ six times and heated to 90° C. The mixture was stirred for 12 hours, cooled to rt, and concentrated under reduced pressure. The crude product was taken up in EtOAc (2 mL) and filtered. The resultant solid was washed with EtOAc (2×2 mL) and H₂O (2×2 mL), then dried under high vacuum to provide 0.25 g (61% yield) of compound 51A as a tan solid. MS (M+H)=550.2.

### Example 52

### Preparation of Intermediate Compound 52A

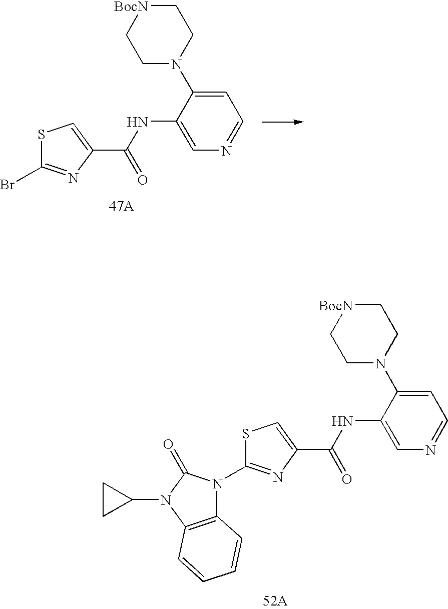

Compound 47A (0.35 g, 0.75 mmol) was reacted with compound 49A (0.12 g, 0.75 mmol) according to the method described in Example 51 to provide 0.28 g (66% yield) of compound 52A as a light yellow solid. MS (M+H)=562.3.

### Example 53

### Preparation of Intermediate Compound 53A

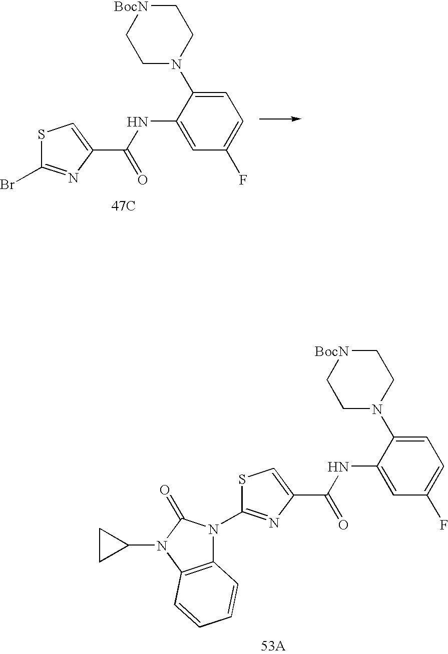

Compound 47C (0.35 g, 0.72 mmol) was reacted with compound 49A (0.13 g, 0.72 mmol) according to the method described in Example 51 to provide 0.28 g (66% yield) of compound 53A as a gray solid. MS (M+H)=579.1.

### Example 54

### Preparation of Intermediate Compound 54A

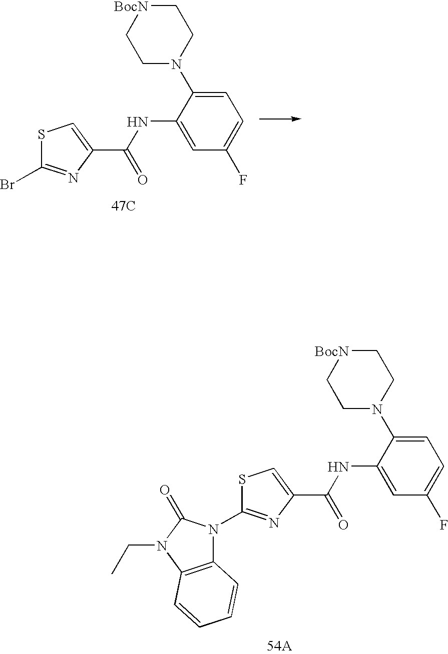

Compound 47C (0.35 g, 0.72 mmol) was reacted with compound 48A (0.12 g, 0.72 mmol) according to the method described in Example 51 to provide 0.29 g (71% yield) of compound 54A as an orange solid. MS (M+H)=567.1.

### Example 55

### Preparation of Intermediate Compound 55A

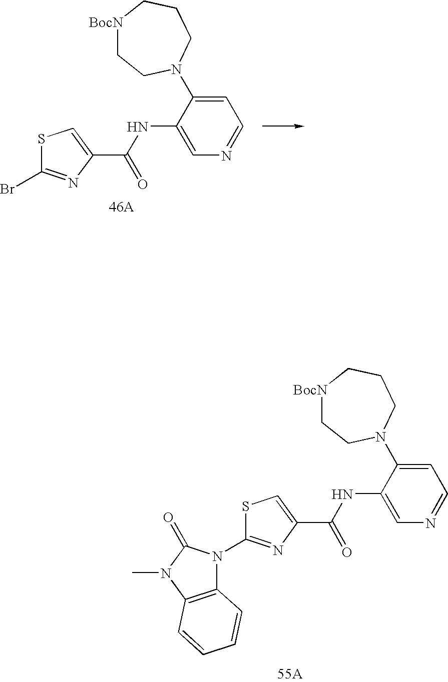

Compound 46A (0.10 g, 0.21 mmol) was reacted with 1-methyl-2-benzimidazolone (31 mg, 0.21 mmol) according to the method described in Example 51 to provide 0.11 g (95% yield) of compound 55A as an orange solid. LC-MS [M+H]=550.3; 99% purity.

### Example 56

### Preparation of Intermediate Compound 56A

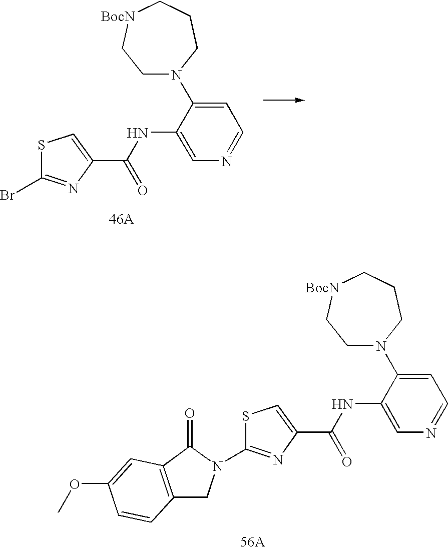

Compound 46A (0.25 g, 0.52 mmol) was reacted 6-methoxyisoindolin-1-one (93 mg, 0.57 mmol) according to the method described in Example 51 to provide 0.28 g (96% yield) of compound 56A as an orange solid. LC-MS [M+H]=565.3; 80% purity.

### Example 57

### Preparation of Intermediate Compound 57A

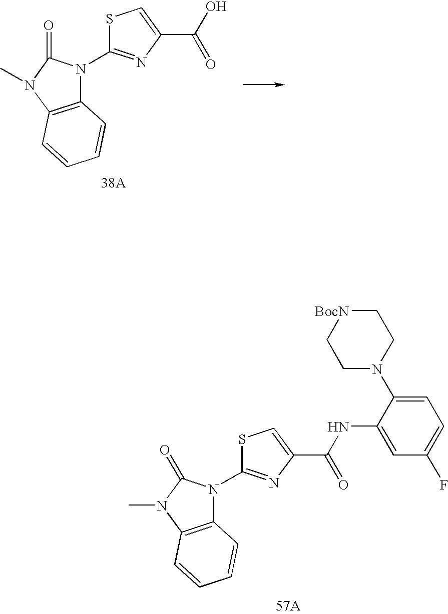

Compound 38A (0.15 g, 0.55 mmol) was reacted with compound 44C (0.12 g, 0.72 mmol) according to the method described in Example 58 below to provide 0.29 g (71% yield) of compound 57A as an orange solid. MS (M+H)=567.1.

### Example 58

### Preparation of Intermediate Compound 58A

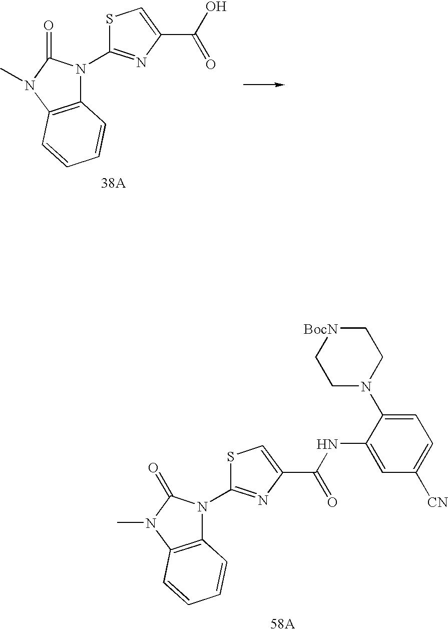

To a solution of compound 38A (0.10 g, 0.36 mmol) in CH₂Cl₂ (4 mL) at room temperature was added oxalyl chloride (61 □L, 0.72 mmol) followed by DMF (3 drops). The mixture was stirred for 1 h whereupon an addition portion of oxalyl chloride (61 □L, 0.72 mmol) and DMF (3 drops) were added. After an additional 1 hours, the mixture was concentrated under reduced pressure and redissolved in CH₂Cl₂ (3 mL). DIPEA (0.19 mL, 1.1 mmol) was added followed by addition of compound 45A (0.12 g, 0.42 mmol) and the mixture was stirred for 12 hours. The reaction mixture was concentrated to dryness and was partitioned between sat. aq NaHCO₃ (3 mL) and CH₂Cl₂ (10 mL). The layers were separated, the aqueous layer was extracted with CH₂Cl₂ (2×10 mL), and the organic layers were combined. The organic layer was washed with brine (1×4 mL), dried (Na₂SO₄), filtered, and concentrated under reduced pressure. The crude product was purified using flash chromatography using a 50:1 mixture of CH₂Cl₂/MeOH as eluent to provide 0.10 (50% yield) of compound 58A as a yellow semisolid. MS [M+H]=560.2.

### Example 59

### Preparation of Intermediate Compound 59A

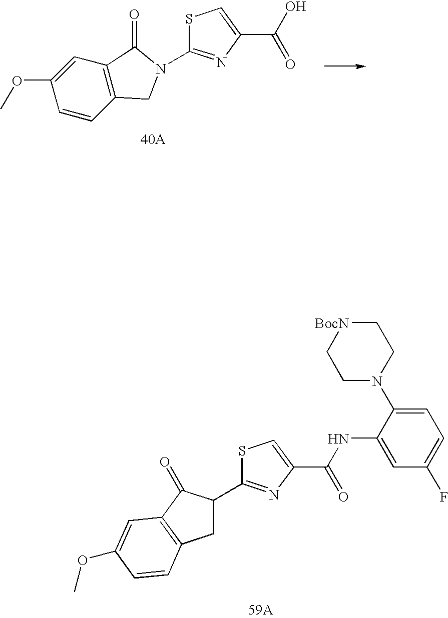

To a solution of compound 40A (0.15 g, 0.52 mmol) in DMF (2 mL) was added compound 44C (0.17 g, 0.57 mmol), followed by N-methylmorpholine (0.17 mL, 1.56 mmol) and PyBop (0.54 g, 1.1 mmol). The resulting mixture was stirred for 72 h at room temperature and concentrated under reduced pressure. The crude residue was taken up in EtOAc (8 mL) and sat. aq NaHCO₃ (3 mL) was added. The layers were separated and the aqueous layer was extracted with EtOAc (2×8 mL). The organic layers were combined and washed with brine (1×5 mL). The organic layer was dried (Na₂SO₄), filtered, and concentrated under reduced pressure. The crude product was purified using preparative thin-layer chromatography using a 2:1 mixture of hexanes/EtOAc as eluent to provide 40 mg (14% yield) of compound 59A a light yellow solid. LC-MS [M+H]=568.3; 89% purity.

### Example 60

### Preparation of Intermediate Compound 60A

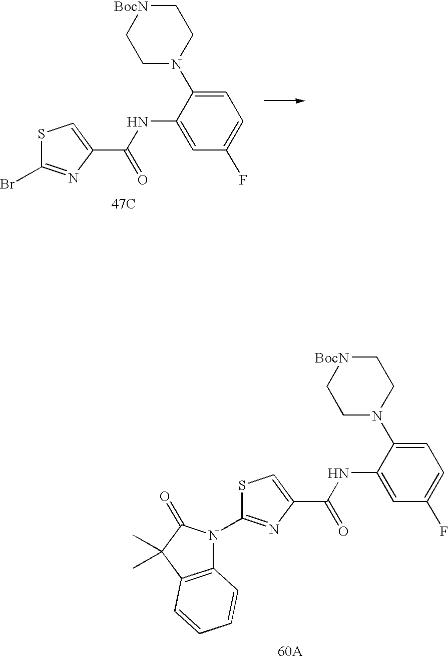

To a solution of compound 47C (0.30 g, 0.61 mmol) was added compound 50A (98 mg, 0.61 mmol), CuI (58 mg, 0.31 mmol), K₃PO₄ (263 mg, 1.24 mmol), and 1,2-trans-diamino cyclohexane (73 □L, 0.61 mmol). The mixture was diluted with dioxane (4 mL) and degassed under house vacuum and filled with N₂ six times. The reaction mixture was heated to 100° C., stirred for 12 hours, cooled to rt, and concentrated under reduced pressure. The crude product was taken up in a 20:1 mixture of CH₂Cl₂/MeOH (5 mL), filtered and concentrated under reduced pressure. The residue from the filtrate was purified using preparative thin-layer chromatography using a 3:1 mixture of hexanes/EtOAc as eluent to provide 125 mg (36% yield) of compound 60A as an off-white solid. LC-MS [M+H]=566.3; 98% purity.

### Example 61

### Preparation of Intermediate Compound 61A

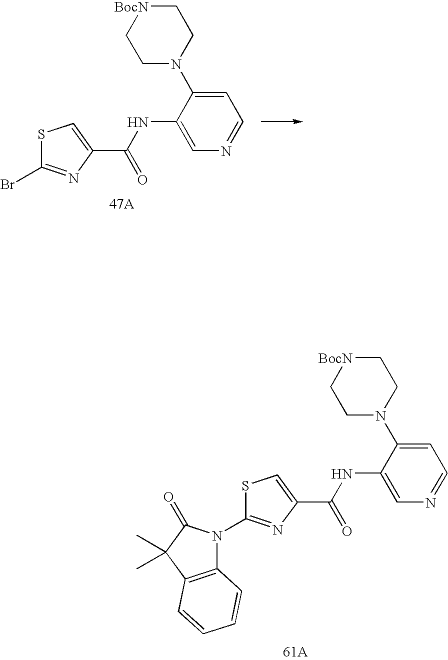

Using the method described in Example 60, compound 47A (0.35 g, 0.75 mmol) was reacted with compound 50A (0.12 g, 0.75 mmol) to provide 0.15 g (36% yield) of compound 61A as a brown solid. LC-MS [M+H]=549.3; 90% purity.

### Example 62

### Preparation of Compound 258

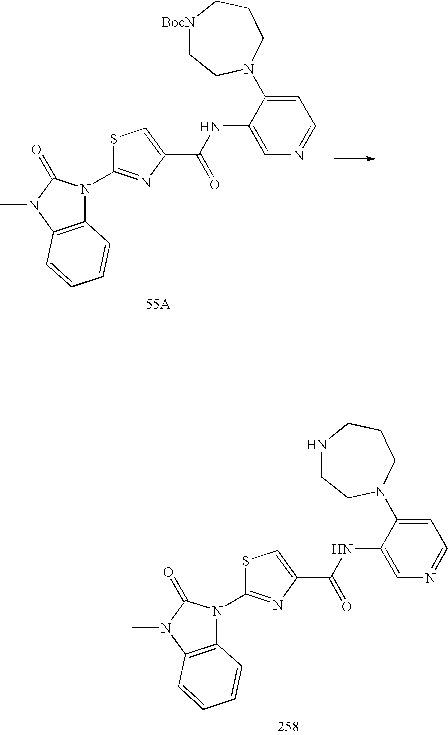

To a solution of compound 55A (42 mg, 0.08 mmol) in CH₂Cl₂ (3 mL) at room temperature was added TFA (1 mL). The mixture was stirred for 3 hours at room temperature and was concentrated under reduced pressure. The crude product was taken up in 7M NH₃ in MeOH (5 mL), stirred for 2 hours, and concentrated under reduced pressure. The crude product was purified using preparative thin-layer chromatography using a 12:1 mixture of CH₂Cl₂/MeOH (7M NH₃) as eluent to provide 29 mg (85% yield) of compound 258 as a pale yellow solid. mp 152-155° C., LC-MS [M+H]=450.1; 95% purity.

### Example 63

### Preparation of Compounds 229-232, 239, 256, 288, 301 and 313

Using the methods described in Examples 60 and 62, and utilizing the Boc adducts indicated, the following illustrative compounds of the invention were made.

| | | 1. Yield (%) |
|---|---|---|
| | | 2. LC-MS |
| Boc adduct | Product | 3. mp (° C.) |
| 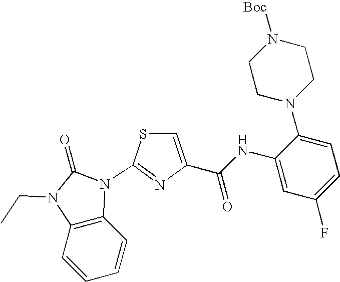 | 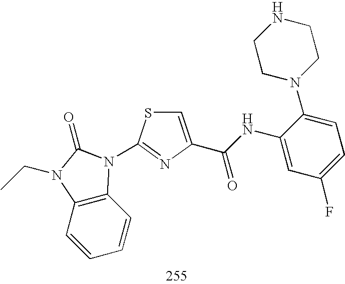 | 1. 90 2. 467.3 3. 242-244 |
| 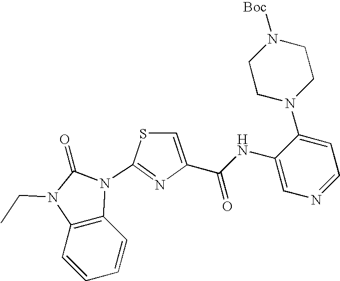 | 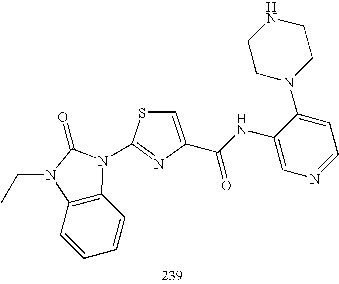 | 1. 73 2. 450.2 3. 172-175 |
| 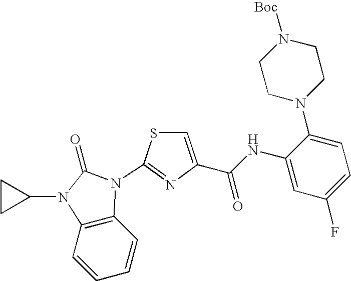 | 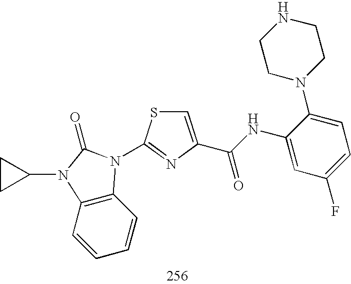 | 1. 92 2. 479.3 3. 220-223 |
| 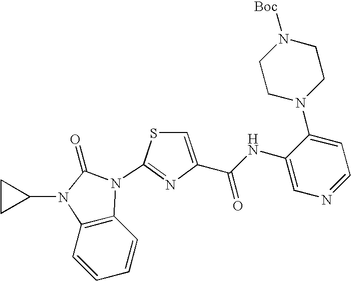 | 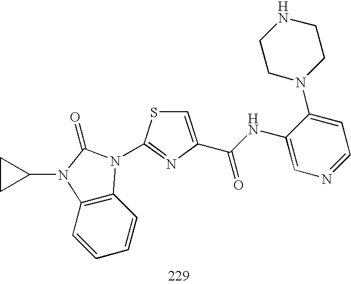 | 1. 67 2. 462.3 3. 180-183 |
| 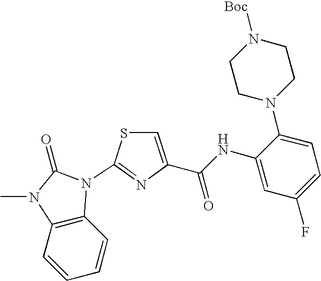 | 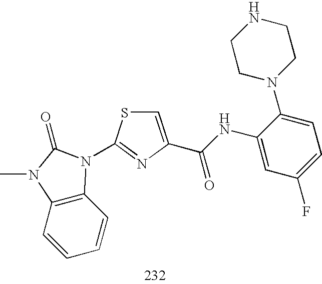 | 1. 62 2. 453.2 3. 140-142 |
| 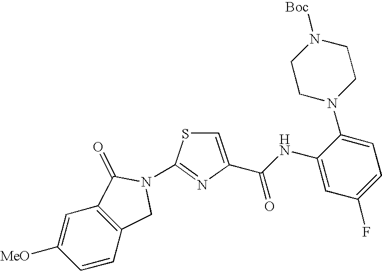 | 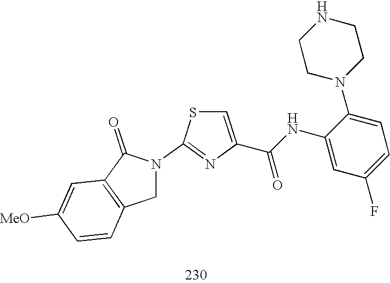 | 1. 86 2. 468.3 3. 168-171 |
| 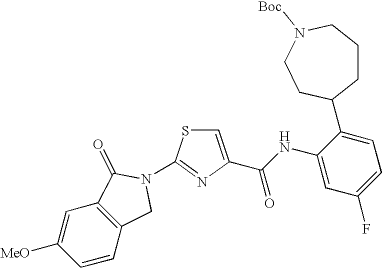 | 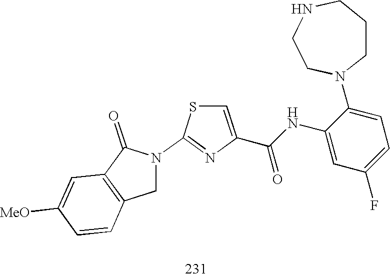 | 1. 90 2. 465.3 3. 133-135 |
| 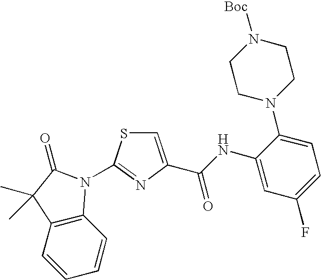 | 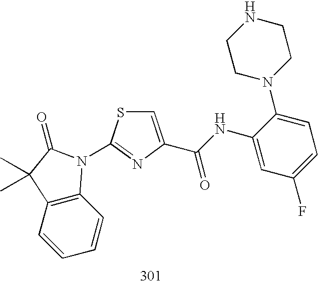 | 1. 87 2. 466.3 3. 167-170 |
| 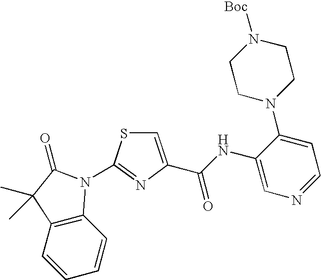 | 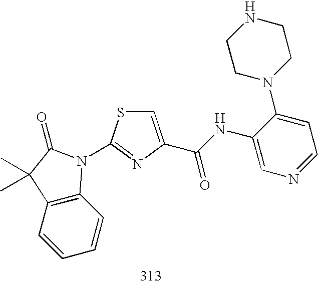 | 1. 92 2. 449.2 3. 200-205 |
| 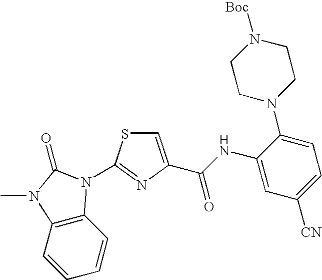 | 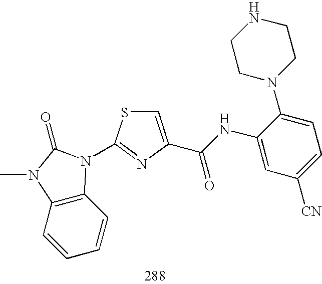 | 1. 57 2. 460.2 3. 168-171 |

### Example 64

### Preparation of Compound 64A

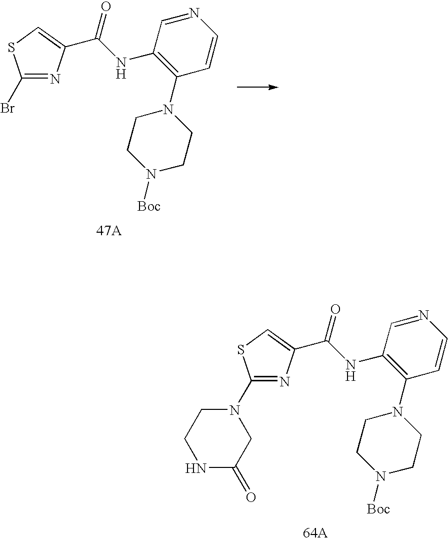

Using the methods described in Examples 60 and 62, compound 47A (0.15 g, 0.32 mmol) was reacted with piperazin-2-one (96 mg, 0.96 mmol) in dioxane (2 mL) to provide 84 mg (54% yield) of compound 64A as a light yellow solid. LC-MS [M+H]=488.3; 98% purity.

### Example 65

### Preparation of Compound 112

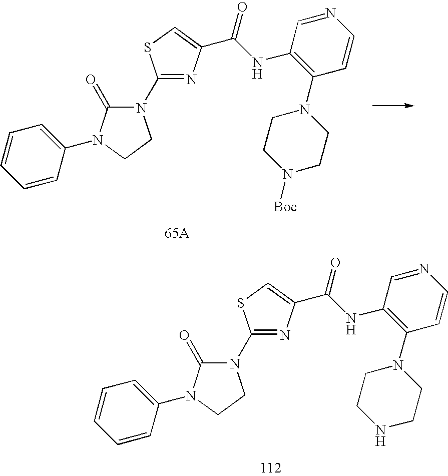

To a solution of compound 65A (0.13 g, 0.22 mmol) in CH₂Cl₂ (3 mL) at room temperature was added TFA (1 mL). The mixture was stirred for 2 hours at room temperature and was concentrated under reduced pressure. The crude product was taken up in 2M ammonia in MeOH (3 mL), stirred for 2 hours, then concentrated under reduced pressure. The crude product was purified using preparative thin-layer chromatography using a 11:1 mixture of CH₂Cl₂/MeOH (7M NH₃) as eluent to provide 63 mg (64% yield) of compound 112a pale yellow solid. mp 116-118° C., LC-MS [M+H]=450.2; 95% purity.

Using the method described above, the indicated Boc adducts were deprotected to provide illustrative compounds 96, 101 and 111 shown in the table below.

| | | 1. Yield (%) |
|---|---|---|
| | | 2. LC-MS |
| Boc adduct | Product | 3. mp (° C.) |
| 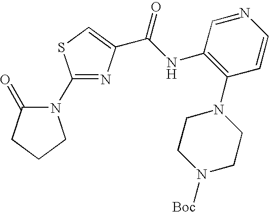 | 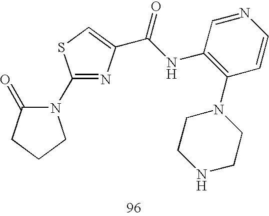 | 1. 45 2. 373.2 3. 122-424 |
| 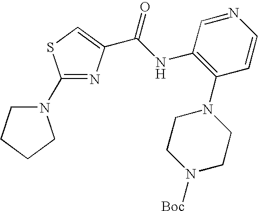 | 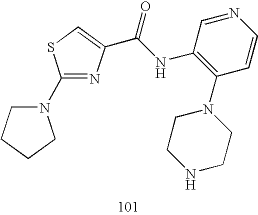 | 1. 71 2. 359.2 3. 179-181 |
| 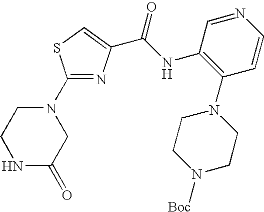 | 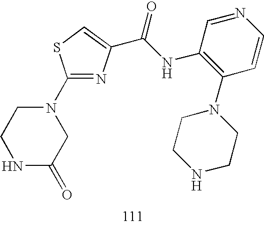 | 1. 77 2. 388.2 3. 215-218 |

### Example 66

### Preparation of Compound 82

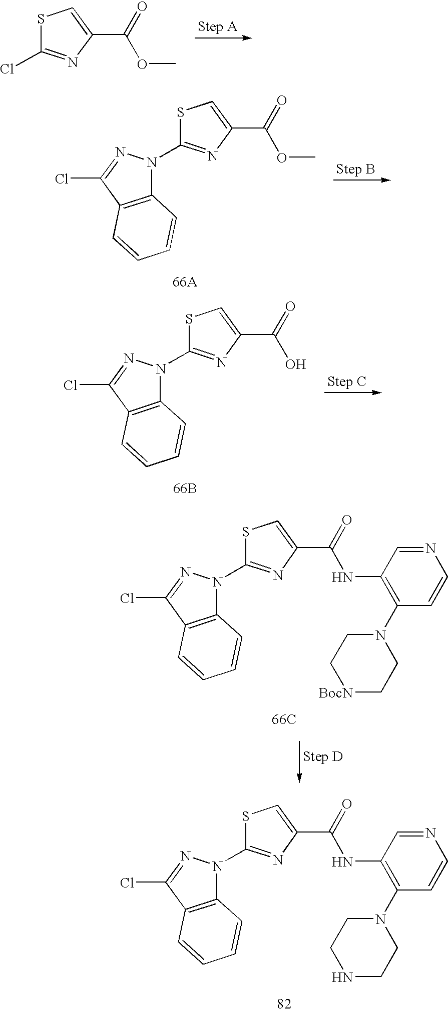

### Step A:

3-Chloroindazole (305 mg, 2.0 mmol) and 2-chlorothiazole (355 mg, 2.0 mmol) were taken up in DMF (20 mL). NaH (80 mg, 60% in oil, 2.0 mmol) was added carefully and the resulting mixture was heated to 60° C. and stirred for 3 hours. After cooling to room temperature, NH₄Cl (aq.) was added carefully and the resulting solution was extracted with EtOAc (60 mL×3). The organics was dried over Na₂SO₄, concentrated under vacuum, and purified using flash column chromatography on silica gel (EtOAc/Hexane=30:70) to provide compound 66A (503 mg) as brown solid. HPLC-MS t_{R}=2.24 min (UV₂₅₄ₙₘ); mass calculated for formula C₁₂H₈ClN₃O₂S 293.0, observed LCMS m/z 294.0 (M+H).

### Step B:

Compound 66A (503 mg, 1.7 mmol) was diluted with THF (10 mL) and to the resulting solution was added LiOH (1N, 3.0 mL). The mixture was stirred at room temperature for about 15 hours. The solvent was removed under vacuum, and the residue obtained was diluted with H₂O (5 mL). 1N HCl was added to adjust the pH to 5 and the solid formed was collected by filtration, then washed with water and air-dried to provide compound 66B, which was used in the next step without further purification. HPLC-MS t_{R}=1.71 min (UV₂₅₄ₙₘ); mass calculated for formula C₁₁H₆ClN₃O₂S 279.0, observed LCMS m/z 280.0 (M+H).

### Step C:

Compound 66C was synthesized from compound 66B using the method described in Example 59. HPLC-MS t_{R}=1.75 min (UV_{254 nm}); mass calculated for formula C₂₅H₂₆ClN₇O₃S 539.2, observed LCMS m/z 540.1 (M+H).

### Step D:

Compound 82 was prepared from compound 66C using the method described in Example 62. HPLC-MS t_{R}=1.10 min (UV_{254 nm}); mass calculated for formula C₂₀H₁₈ClN₇OS 439.1, observed LCMS m/z 440.0 (M+H).

### Example 67

### Preparation of Compound 78

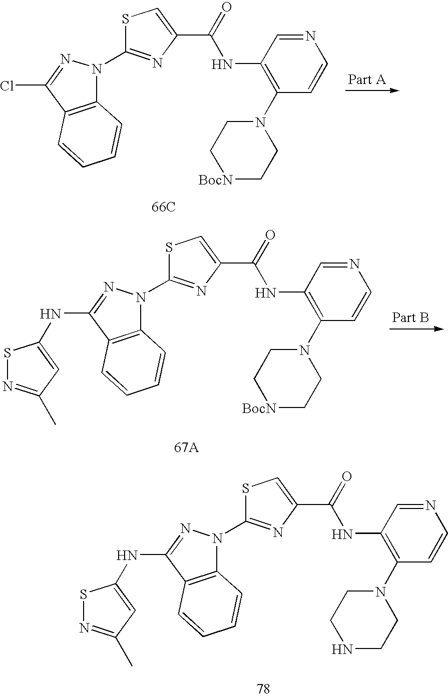

### Step A:

To a 25 ml round bottom flask charged with compound 66C (270 mg, 0.5 mmol), isothiazole HCl salt (300 mg, 2.0 mmol), Pd₂(dba)₃ (45 mg, 0.05 mmol), 2-di-t-butylphosphino-2′,4′,6′-tri-1-propyl-1,1-biphenyl (42 mg, 0.1 mmol) and K₃PO₄ (616 mg, 3.0 mmol) was added toluene (10 mL). The mixture was thoroughly degassed by alternately connected the flask to vacuum and Argon. This resulting mixture was then heated to 90° C. and stirred for about 15 hours, then was diluted by EtOAc (40 mL) and washed with brine. After concentration, the residue obtained was purified using Preparative liquid chromatography to provide compound 67A. HPLC-MS t_{R}=1.49 min (UV_{254 nm}); mass calculated for formula C₂₉H₃₁N₉O₃S₂ 617.2, observed LCMS m/z 618.1 (M+H).

### Step B:

Compound 78 was prepared by removing the Boc protecting group from compound 67A using the method described in Example 62. HPLC-MS t_{R}=0.97 min (UV_{254 nm}); mass calculated for formula C₂₄H₂₃N₉OS₂ 517.1, observed LCMS m/z 518.1 (M+H).

### Example 68

### Preparation of Compound 90

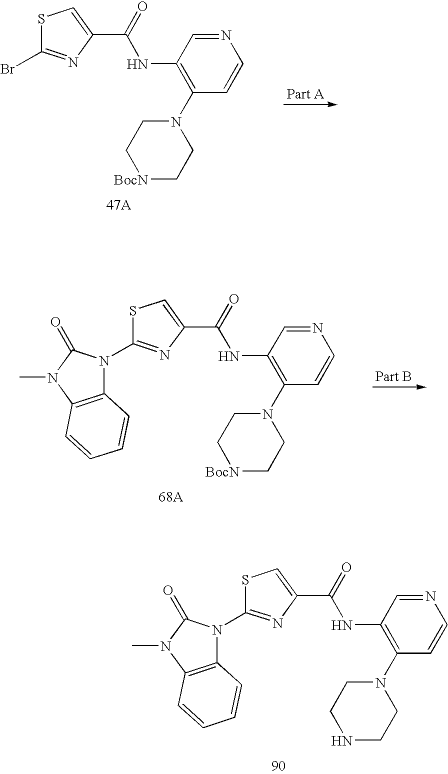

### Step A:

To a 25 mL round bottom flask charged with compound 47A (100 mg, 0.22 mmol), benzimidazolone (45 mg, 0.3 mmol), CuI (10 mg, 0.05 mmol), trans-1,2-dimethylaminocyclohexane (13 mg, 0.1) and K₂CO₃ (51 mg, 0.3 mmol) was added dioxane (10 mL). The mixture was thoroughly degassed by alternately connected the flask to vacuum and Argon. This resulting mixture was then heated at 80° C. for about 15 hours. After cooling to room temperature, the solvent was removed under vacuum. The residue obtained was diluted with H₂O (5 mL) and the crude solid compound 68A was collected by filtration and used directly in the next step without further purification. HPLC-MS t_{R}=1.42 min (UV_{254 nm}); mass calculated for formula C₂₆H₂₉N₇O₄S 535.2, observed LCMS m/z 536.2 (M+H).

### Step B:

Compound 90 was prepared by removing the Boc protecting group from compound 68A using the method described in Example 62. HPLC-MS t_{R}=0.83 min (UV_{254 nm}); mass calculated for formula C₂₁H₂₁N₇O₂S 435.1, observed LCMS m/z 436.1 (M+H).

Using the method described in Steps A and B above, and using the appropriate coupling partners in Step A, the following illustrative compounds were made.

| | MS | |
|---|---|---|
| | m/z | HPLC |
| Compound | (M + H) | MS t_{R} |
| 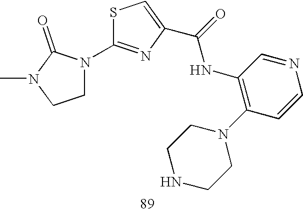 | 388.1 | 0.74 |
| 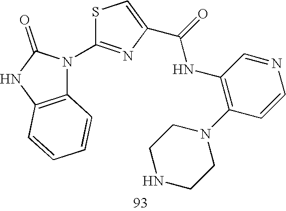 | 422.1 | 0.71 |
| 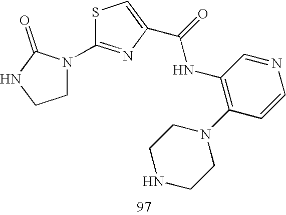 | 374.2 | 0.47 |
| 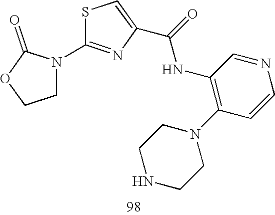 | 375.1 | 0.55 |
| 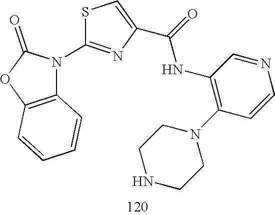 | 423.1 | 0.88 |
| 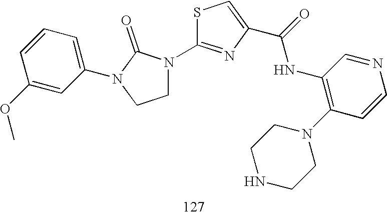 | 480.1 | 1.00 |
| 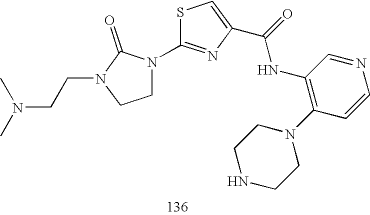 | 493.1 | 0.58 |
| 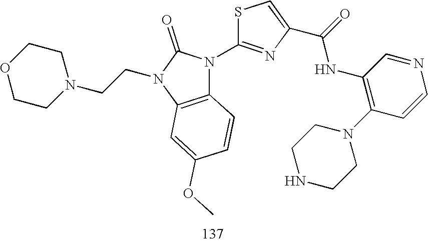 | 535.1 | 0.61 |
| 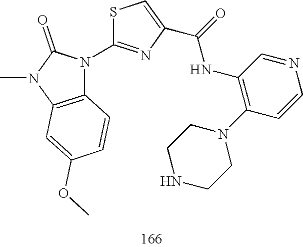 | 466.1 | 0.86 |
| 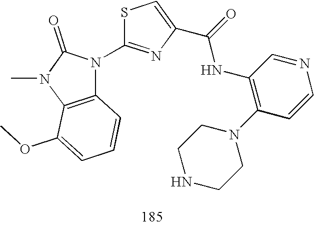 | 466.1 | 0.97 |
| 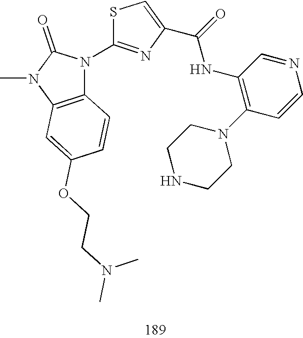 | 523.1 | 0.63 |
| 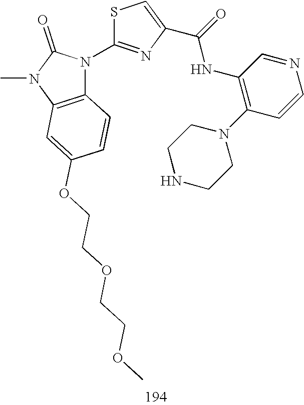 | 554.2 | 0.93 |
| 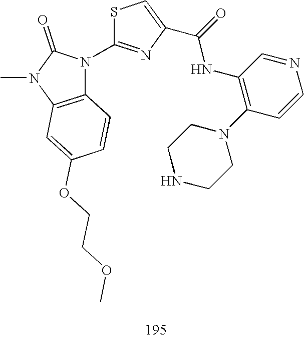 | 510.1 | 0.90 |
| 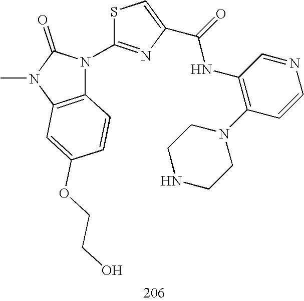 | 496.2 | 0.97 |
| 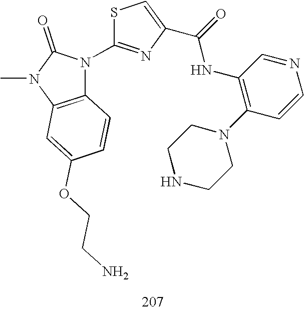 | 495.1 | 0.68 |
| 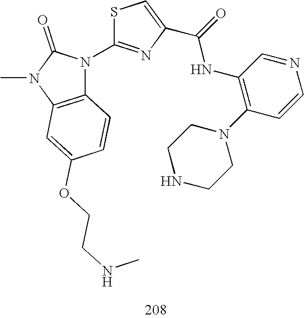 | 509.2 | 0.69 |
| 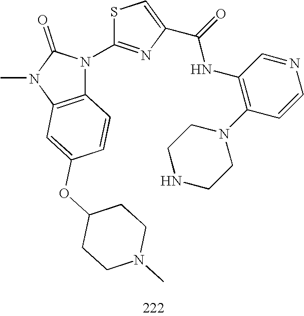 | 549.2 | 0.75 |
| 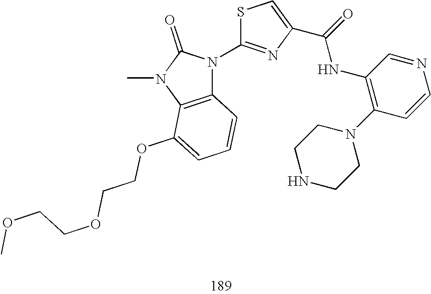 | 554.2 | 1.15 |
| 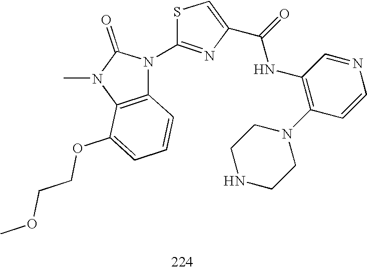 | 510.2 | 1.13 |
| 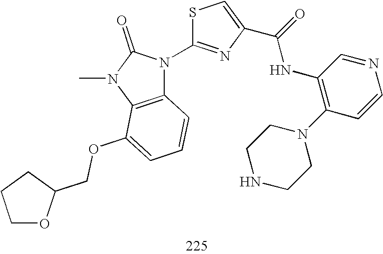 | 536.2 | 1.21 |
| 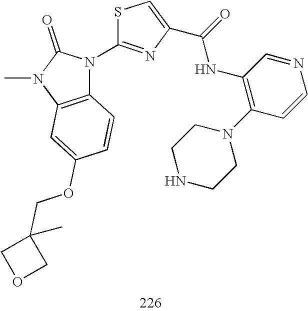 | 536.2 | 1.17 |
| 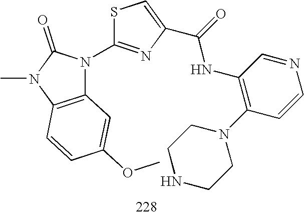 | 466.1 | 0.91 |
| 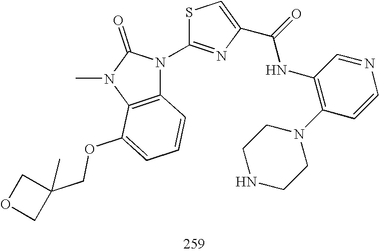 | 536.3 | 0.98 |
| 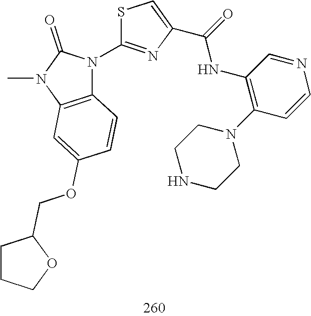 | 536.2 | 1.23 |
| 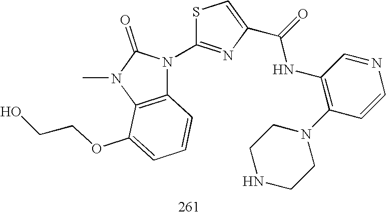 | 496.1 | 0.90 |
| 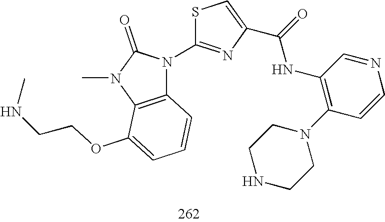 | 509.2 | 0.67 |
| 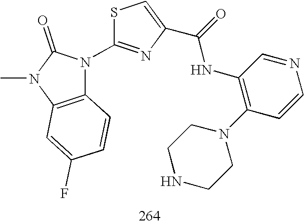 | 454.1 | 1.02 |
| 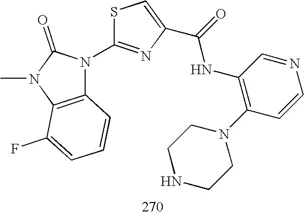 | 454.1 | 0.93 |
| 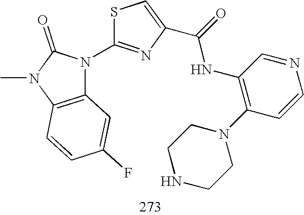 | 454.1 | 1.02 |
| 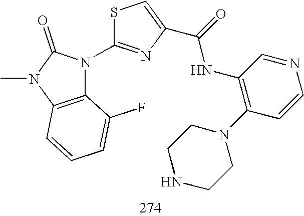 | 454.1 | 1.02 |
| 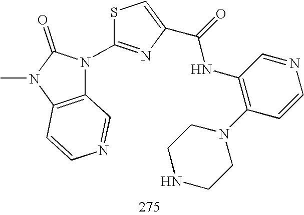 | 437.2 | 0.64 |
| 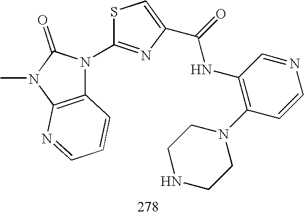 | 437.2 | 0.93 |
| 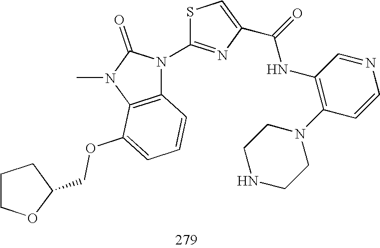 | 536.2 | 1.22 |
| 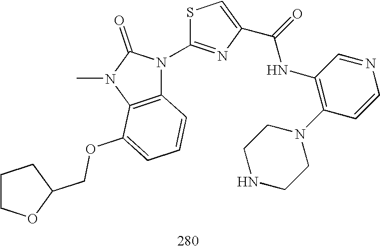 | 536.2 | 1.25 |
| 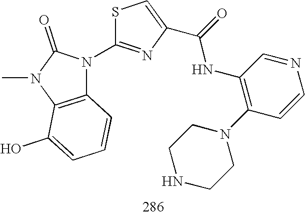 | 452.2 | 1.12 |
| 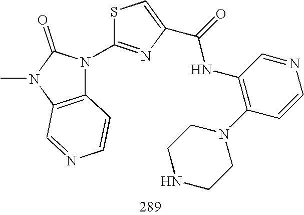 | 437.1 | 0.19 |
| 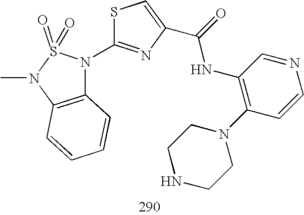 | 472.1 | 0.94 |
| 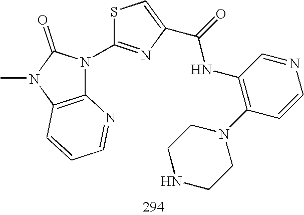 | 437.0 | 0.92 |
| 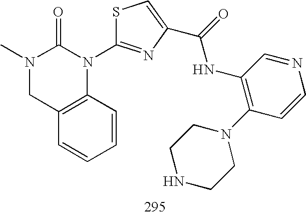 | 450.1 | 1.06 |
| 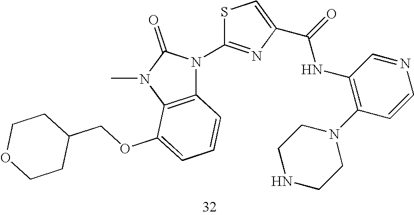 | 550.2 | 1.09 |
| 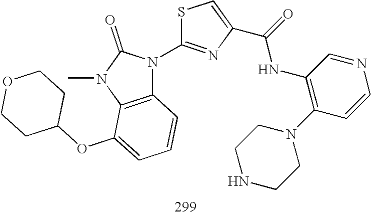 | 536.2 | 1.06 |
| 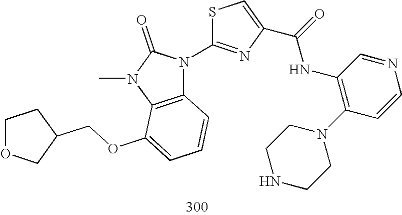 | 536.2 | 1.06 |
| 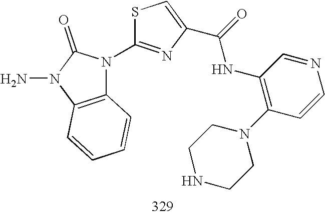 | 437.1 | 0.72 |
| 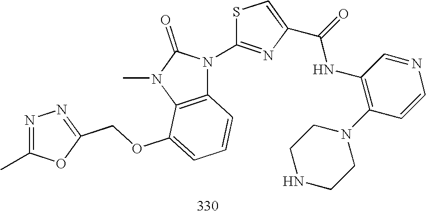 | 548.2 | 1.11 |
| 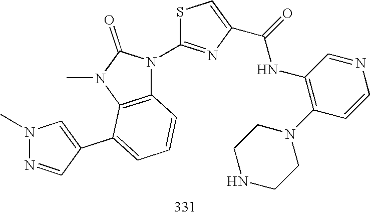 | 516.1 | 0.93 |
| 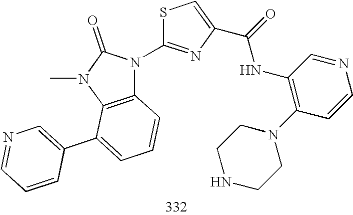 | 513.1 | 0.92 |

### Example 69

### Preparation of Compound 69B

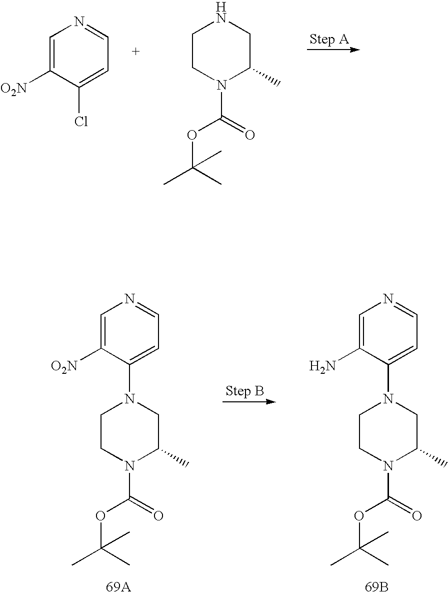

### Step A:

A solution of 4-chloro-3-nitro-pyridine (2.0 mmol, 0.32 g), diethylisopropyl amine (3.0 mmol, 0.52 mL) and 2(S)-methyl-piperazine-1-carboxylic acid tert-butyl ester (2.5 mmol, 0.50 g) in dioxane (2 mL) was irradiated using microwave for 20 minutes at a temperature of 120° C. The reaction mixture was concentrated in vacuo, and the resulting residue was purified using flash column chromatography on silica gel (eluent: ethyl acetate) to provide compound 69A as a yellow solid in quantitative yield. HPLC-MS RT=1.42 min, mass calculated for formula C₁₅H₂₂N₄O₄ 322.16, observed LCMS m/z 323.1 (M+H).

### Step B:

To a solution of compound 69A (600 mg) in Ethanol/EtOAc (1:1, 10 mL) was added Pd on carbon (5% Pd). The reaction mixture was stirred under a hydrogen atmosphere at room temperature for about 15 hours, then filtered through a pad of celite. The filtrate was concentrated. in vacuo to provide Compound 69B as a solid. HPLC-MS RT=1.10 min, mass calculated for formula C₁₅H₂₄N₄O₂ 292.19, observed LCMS m/z 293.20 (M+H).

Using the methods described in Steps A and B above, and using the appropriate reactants, the following intermediate compounds were made:

| | M + H | Retention |
|---|---|---|
| Compound | Observed | Time |
| 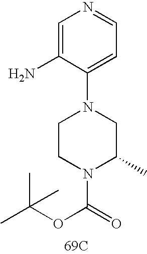 | 293.2 | 1.10 |
| 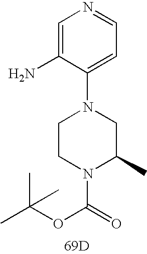 | 293.2 | 1.10 |
| 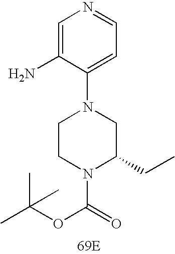 | 307.3 | 1.0 |
| 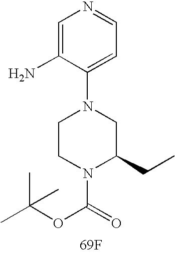 | 307.3 | 1.0 |
| 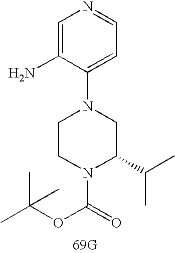 | 321.2 | 1.15 |
| 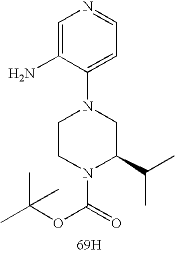 | 321.2 | 1.15 |
| 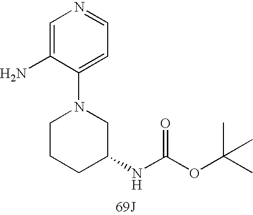 | 293.2 | 0.09 |
| 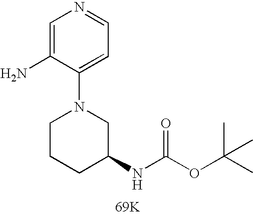 | 293.2 | 0.09 |
| 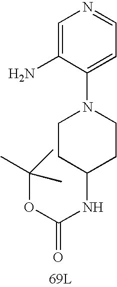 | 293.3 | 1.0 |
| 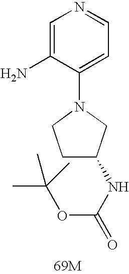 | 279.1 | 0.85 |
| 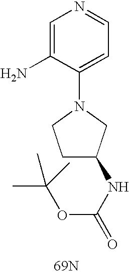 | 279.1 | 0.85 |
| 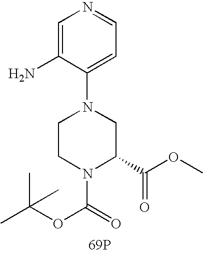 | 337.12 | 0.95 |
| 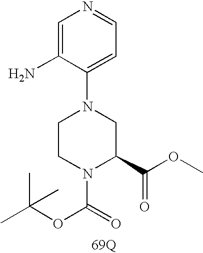 | 337.12 | 0.95 |
| 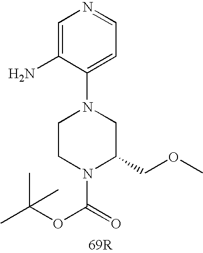 | 323.2 | 1.05 |

### Example 70

### Preparation of Compound 70B

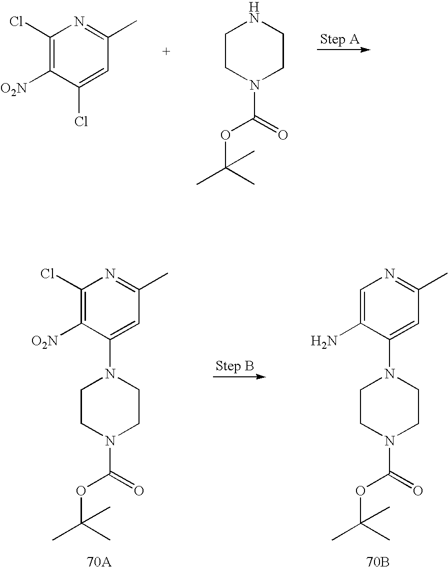

### Step A:

A solution of 2,4-dichloro-6-methyl-3-nitro-pyridine (2.0 mmol, 0.42 g), diethylisopropyl amine (3.0 mmol, 0.52 mL) and piperazine-1-carboxylic acid tert-butyl ester (2. mmol, 0.372 g) in dichloromethane (5 mL) was stirred at room temperature for 12 hours. The reaction mixture was concentrated in vacuo, and the resulting residue was purified using flash column chromatography on silica gel (eluent: Hexane and ethyl acetate) to provide compound 70A as a yellow solid in quantitative yield. HPLC-MS RT=2.1 min, mass calculated for formula C₁₅H₂₁ClN₄O₄ 356.13, observed LCMS m/z 357.1 (M+H).

### Step B:

To a solution of compound 70A (600 mg) in Ethanol/EtOAc (1:1, 10 mL) was added Pd on carbon (5% Pd). The reaction mixture was stirred under a hydrogen atmosphere at 40 psi for about 15 hours, then filtered through a pad of celite. The filtrate was concentrated. in vacuo to provide Compound 70B as a solid. HPLC-MS RT=1.10 min, mass calculated for formula C₁₅H₂₄N₄O₂ 292.19, observed LCMS m/z 293.20 (M+H).

Using the methods described in Steps A and B above, and using the appropriate reactants, the following intermediate compounds were made:

| | | | Re- |
|---|---|---|---|
| Starting | | M + H | tention |
| material | Product | Observed | Time |
| 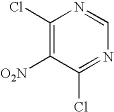 | 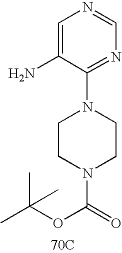 | 280.3 | 0.9 |
| 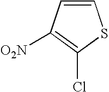 | 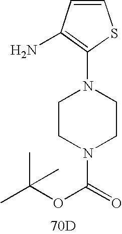 | 284.1 | 1.5 |
| 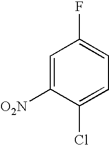 | 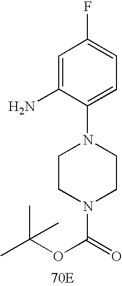 | 296.2 | 2.0 |
| 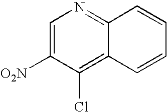 | 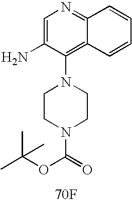 | 329.2 | 1.2 |

### Example 71

### Preparation of Compound 71D

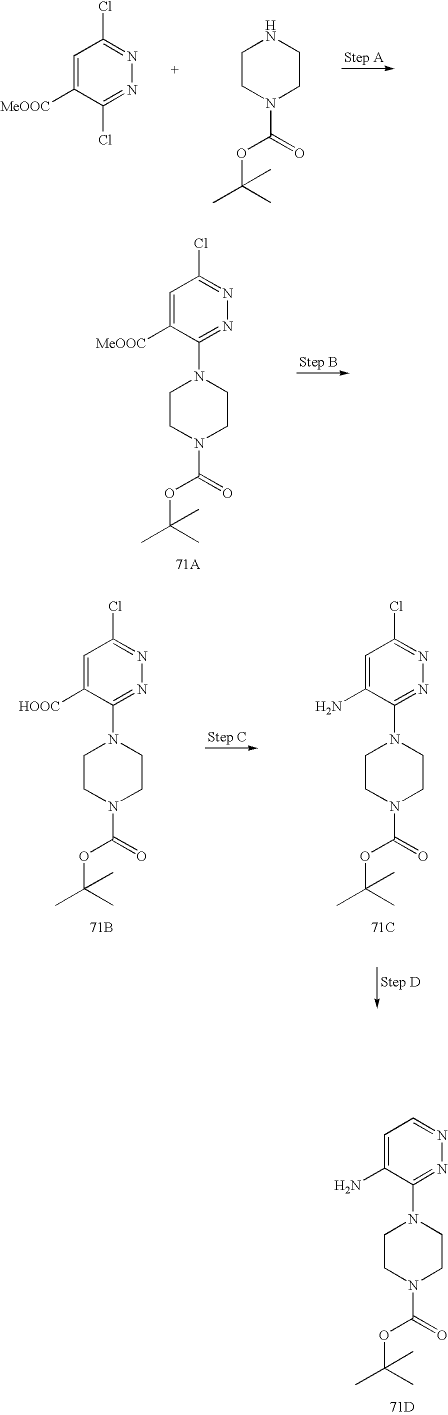

### Step A:

A solution of 3,6-dichloro pyridazine-4-carboxylic acid methyl ester (2.0 mmol, 0.41 g), diethylisopropyl amine (3.0 mmol, 0.52 mL) and piperazine-1-carboxylic acid tert-butyl ester (2 mmol, 0.37 g) in dioxane (2 mL) was irradiated using microwave for 20 minutes at a temperature of 80° C. The reaction mixture was concentrated in vacuo, and the resulting residue was purified using flash column chromatography on silica gel (eluent: ethyl acetate) to provide compound 71A as a yellow solid in quantitative yield. HPLC-MS RT=1.9 min, mass calculated for formula C₁₅H₂₁ClN₄O₄ 356.13, observed LCMS m/z 357.1 (M+H).

### Step B:

To a solution of compound 71A (2.0 mmol. 0.714 g) in 4 ml of THF was added 1N. LiOH solution in water 4 mL and stirred for about 15 hours at room temperature. THF is removed and acidified to pH to 2. Aqueous layer is extracted with ethyl acetate, washed with brine, dried over anhydrous sodium sulfate. Filtered and concentrated to get compound 71B. HPLC-MS RT=1.3 min, mass calculated for formula C₁₄H₁₉ClN₄O₄ 342.11, observed LCMS m/z 343.1 (M+H).

### Step C:

To a solution of Compound 71B (1 mmol, 0.34 g.) in DMF (5 mL) was added DPPA (1 mmol, 0.275 g.) and triethylamine (1.1 mmol, 0.16 mL) and stirred under Ar for 4 hours then added 1 ml of water and heated to 65° C. for 1 hour. Cooled to room temperature and pH was adjusted to 9 by adding potassium carbonate. Extracted with Ethyl acetate, washed with brine, dried over sodium sulfate, filtered and concentrated to obtain compound 71C. HPLC-MS RT=1.35 min, mass calculated for formula C₁₃H₂₀ClN₅O₂ 313.13, observed LCMS m/z 314.2 (M+H).

### Step D:

To a solution of compound 71C (150 mg) in Ethanol/EtOAc (1:1, 10 mL) was added Pd on carbon (5% Pd). The reaction mixture was stirred under a hydrogen atmosphere at 40 psi for about 15 hours, then filtered through a pad of celite. The filtrate was concentrated. in vacuo to provide Compound 71D as a solid. HPLC-MS RT=1.0 min, mass calculated for formula C₁₃H₂₁N5O₂ 279.17, observed LCMS m/z 280.30 (M+H).

### Example 72

### Preparation of Compound 72B

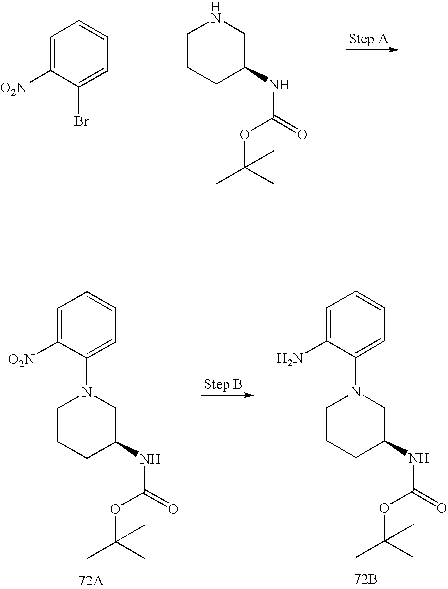

### Step A:

A solution of 1-bromo-2-nitro-benzene (2.0 mmol, 0.4 g), diethylisopropyl amine (3.0 mmol, 0.52 mL) and Compound (2.5 mmol, 0.50 g) in dimethylacetamide (2 mL) was irradiated using microwave for 30 minutes at a temperature of 200° C. The reaction mixture was concentrated in vacuo, and the resulting residue was purified using flash column chromatography on silica gel (eluent: ethyl acetate) to provide compound 72A as a solid. HPLC-MS RT=2.15 min, mass calculated for formula C₁₆H₂₃N₃O₄ 321.17, observed LCMS m/z 322.2 (M+H).

### Step B:

To a solution of compound 72A (400 mg) in Ethanol/EtOAc (1:1, 10 mL) was added Pd on carbon (5% Pd). The reaction mixture was stirred under a hydrogen atmosphere at room temperature for about 15 hours, then filtered through a pad of celite. The filtrate was concentrated in vacuo to provide Compound 72B as a solid. HPLC-MS RT=1.70 min, mass calculated for formula C₁₆H₂₅N3O₂ 291.19, observed LCMS m/z 292.20 (M+H).

Using the methods described in Steps A and B above, and utilizing the enantiomer of compound 72B, the following intermediate compound was made:

| | M + H | Retention |
|---|---|---|
| Compound | Observed | Time |
| 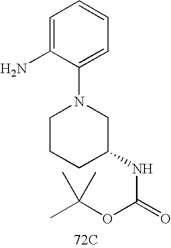 | 292.20 | 1.70 |

### Example 73

### Preparation of Intermediate Compounds 73A-73Q

Using the methods described in Example 46 above, and using the appropriate reactants, the following intermediate compounds were made:

| | M + H |
|---|---|
| Compound | Observed |
| 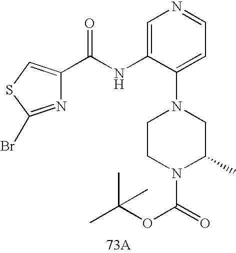 | 482.1 |
| 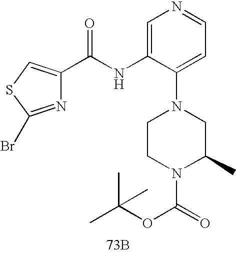 | 482.1 |
| 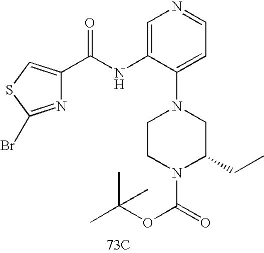 | 496.1 |
| 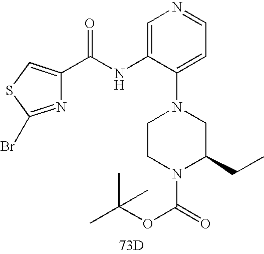 | 496.1 |
| 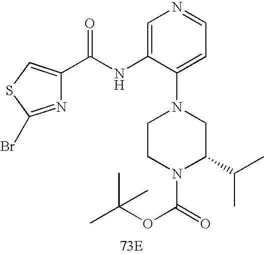 | 510.1 |
| 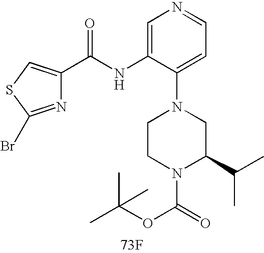 | 510.1 |
| 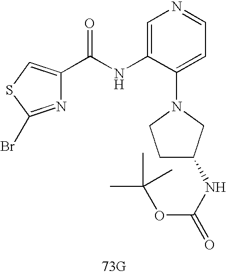 | 468.1 |
| 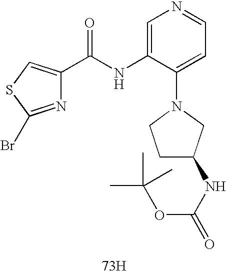 | 468.1 |
| 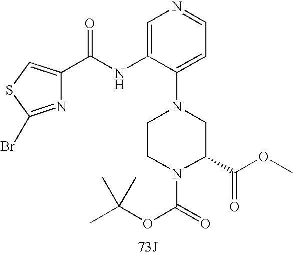 | 526.1 |
| 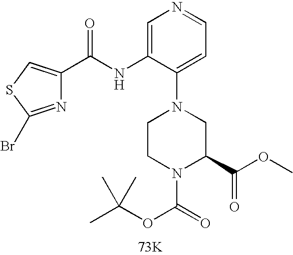 | 526.1 |
| 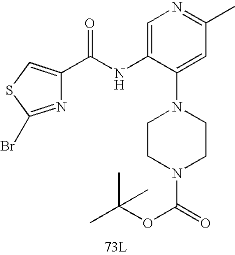 | 482.1 |
| 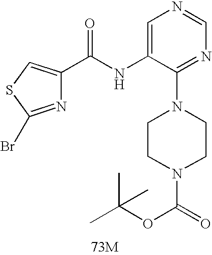 | 469.1 |
| 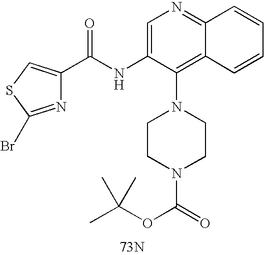 | 518.1 |
| 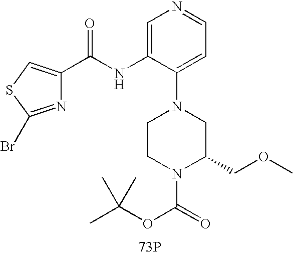 | 512.1 |
| 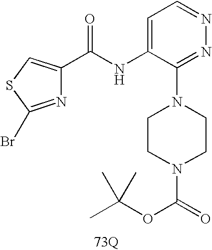 | 469.1 |

### Example 74

### Preparation of Compound 74A

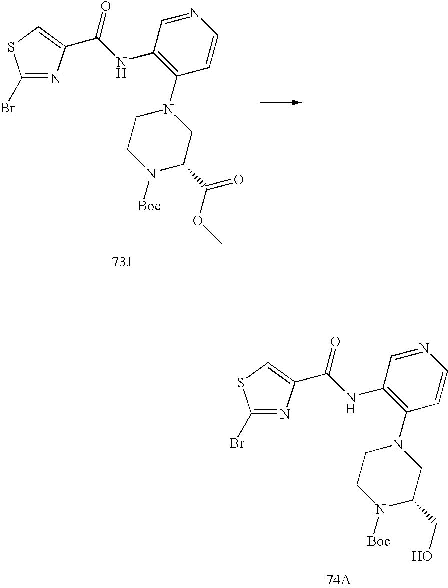

To a solution of compound 73J (0.2 mmol, 0.1 g.) in Ethanol 2 mL added sodium borohydride (0.8 mmol, 0.03 g.) and stirred for about 15 hours. Water is added and extracted with ethylacetate. Ethylacetate layer is washed with water, brine, dried over anhydrous sodium sulfate, filtered and concentrated in vacuo to provide product 75A. RT=1.10 min, mass calculated for formula C₁₉H₂₄BrN₅O₄S 497.07, observed LCMS m/z 498.1 (M+H).

Using this procedure, compound 74B was synthesized from compound 73K.

| | M + H |
|---|---|
| Compound | Observed |
| 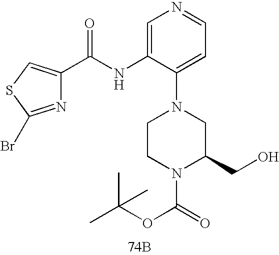 | 498.31 |

### Example 75

### Preparation of Compounds 65, 71, 75, 81, 83, 86, 87, 151-153, 201, 202, 240, 241, 257, 317, 320-322 and 324

The compounds depicted below were made using the method described in Example 51 and utilizing the appropriate reactants.

| | M + H | Retention |
|---|---|---|
| Compound | Observed | Time |
| 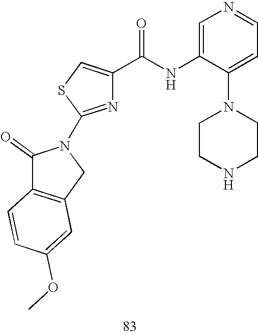 | 451.14 | 2.07 |
| 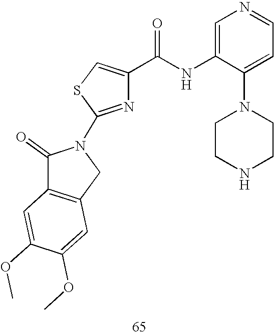 | 467.2 | 0.5 |
| 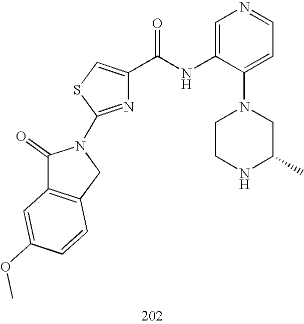 | 465.1 | 0.75 |
| 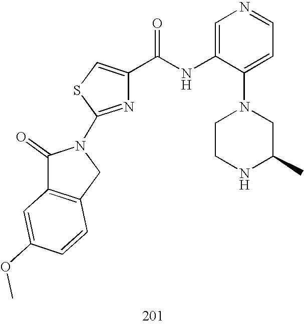 | 465.1 | 0.8 |
| 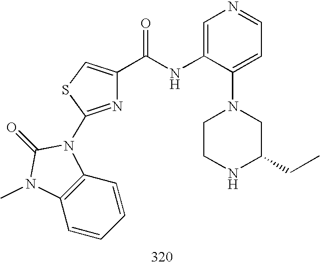 | 464.17 | 1.81 |
| 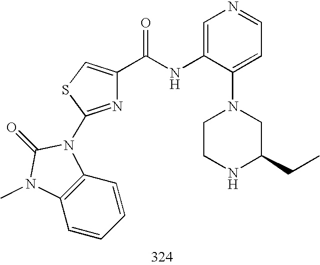 | 464.17 | 1.92 |
| 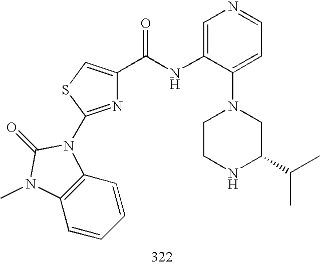 | 478.19 | 1.93 |
| 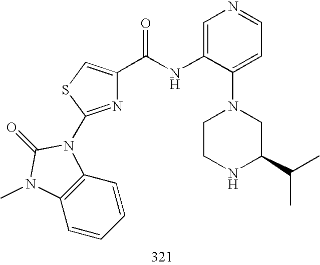 | 478.19 | 1.86 |
| 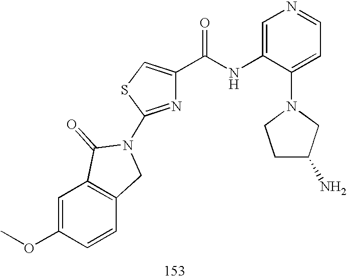 | 451.1 | 2.1 |
| 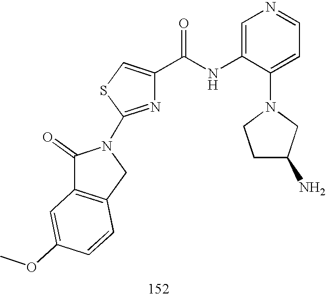 | 451.1 | 2.1 |
| 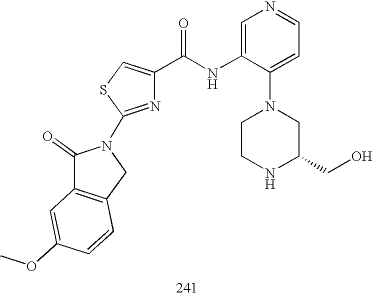 | 481.15 | 1.71 |
| 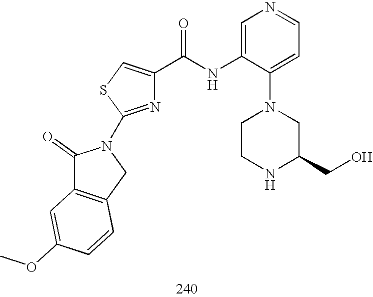 | 481.15 | 1.74 |
| 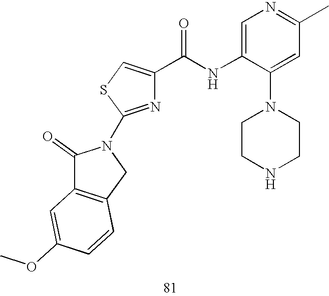 | 465.16 | 2.14 |
| 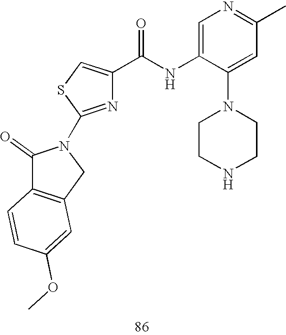 | 465.16 | 1.93 |
| 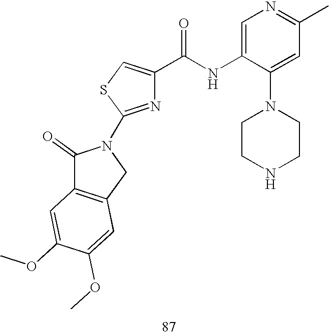 | 495.17 | 1.99 |
| 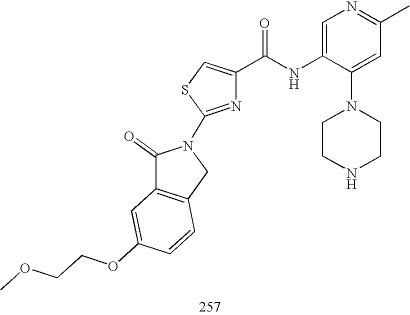 | 509.19 | 2.10 |
| 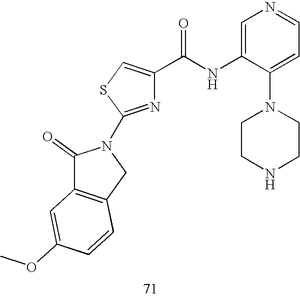 | 452.1 | 2.45 |
| 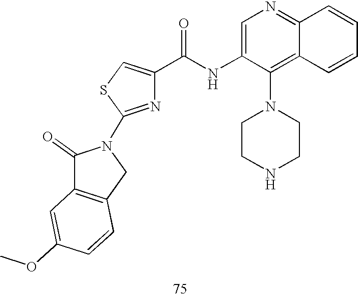 | 501.16 | 2.14 |
| 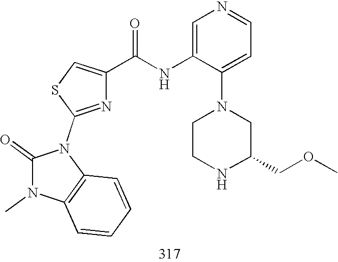 | 480.17 | 1.78 |
| 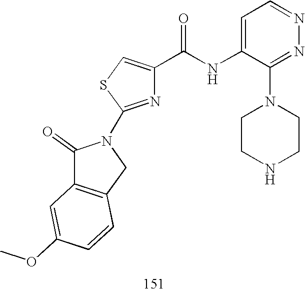 | 452.15 | 2.76 |

### Example 76

### Preparation of Compound 76A and 76B

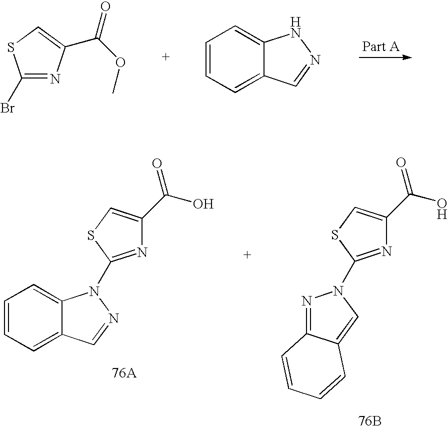

To a suspension of NaH (60% dispersion in oil, 10 mmol, 0.48 g.) in anhydrous dioxane (5 mL) was added a solution of indazole (10 mmol, 1.18 g) in dioxane (5 mL) and the resulting reaction was allowed to stir for 30 minutes. A solution of 2-bromo-thiazole-4-carboxylic acid methyl ester (10.0 mmol, 2.22 g) in dioxane (5 mL) was then added dropwise and the reaction mixture was heated to 100° C. for 4 hrs. The reaction mixture was cooled to room temperature, quenched with water, and the solution was adjusted to pH 2 using 1N HCl. The resulting basic solution was extracted with ethyl acetate and the organic phase was washed with water and brine, dried over anhydrous sodium sulfate, then filtered and concentrated. LCMS of the resulting residue showed two peaks for the acid indicating the formation of two regioisomers (Compounds 76A and 76B). HPLC-MS RT=1.35 and 1.45 min, mass calculated for formula C₁₁H₇N₃O₂S 245.03, observed LCMS m/z 246.1 (M+H).

### Example 77

### Preparation of Compounds 33, 40, 53, 59, 60, 76, 173, 176 and 179

The compounds depicted below were made by reacting compound 76A or 76B with the appropriate coupling partner using the method described in Steps C and D of Example 66.

| | M + H | Retention |
|---|---|---|
| Compound | Observed | Time |
| 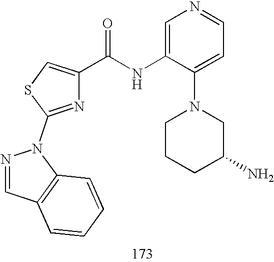 | 420.1 | 2.07 |
| 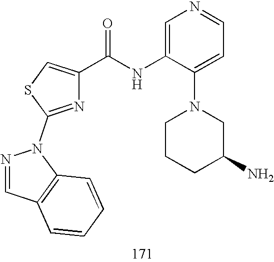 | 420.1 | 2.07 |
| 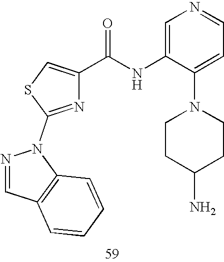 | 420.15 | 2.22 |
| 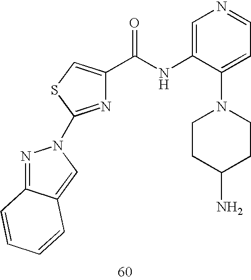 | 420.15 | 2.16 |
| 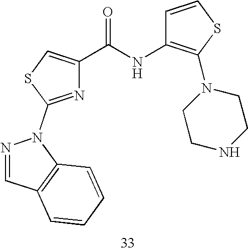 | 411.09 | 3.34 |
| 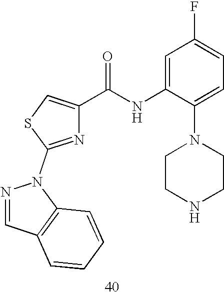 | 423.1 | 3.75 |
| 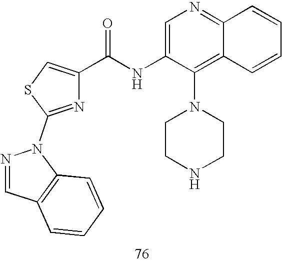 | 456.15 | 2.31 |
| 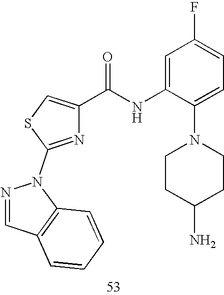 | 437.2 | 4.0 |
| 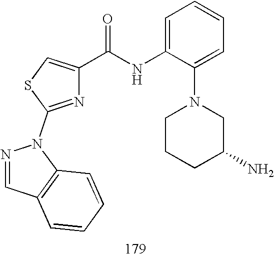 | 419.15 | 3.57 |
| 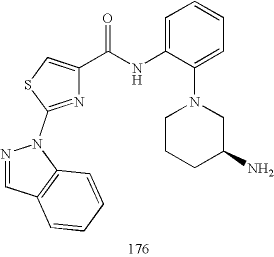 | 419.15 | 3.54 |

### Example 18

### Preparation of Compound 78B

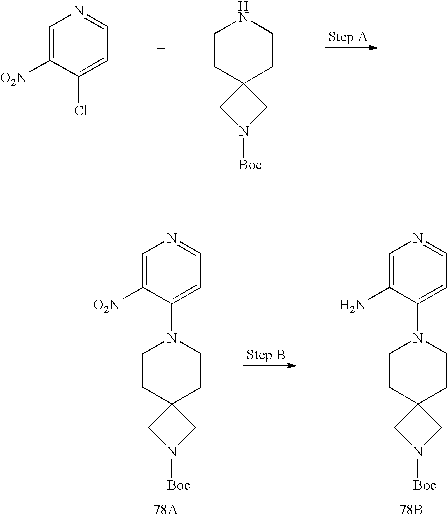

### Step A:

To a vial were added 4-chloro-3-nitro pyridine (2 mmol) and spirocyclic amine (2 mmol). The starting materials were dissolved in 4 mL of dichloromethane followed by the addition of DIPEA (6 mmol). The reaction was stirred at 60° for about 15 hours, then the reaction mixture was concentrated and using preparative liquid chromatography (0-5% methanol in ethyl acetate) to provide compound 78A. Recovered 1.6 mmol (80%) of 274. Mass calculated for formula C₁₇H₂₄N₄O₄ 348.18, observed LCMS m/z 348.20 (M+H).

### Step B:

To a round-bottom flask was added a solution of compound 78A in ethyl acetate. Next, Pd/C was added to the mixture. The flask was sealed with a septum and evacuated. The mixture was hydrogenated using a balloon for about 15 hours. Product was confirmed by LCMS. The Pd/C was filtered off using Celite and the filtrate was concentrated in vacuo to provide compound 78B in quantitative yield. Mass calculated for formula C₁₇H₂₆N₄O₂ 318.21, observed LCMS m/z 319.20 (M+H). LCMS calculated for 275: 318.21.

Using the methods described in Steps A and B above, and using the appropriate reactants, the following intermediate compounds were made:

| | | | Retention |
|---|---|---|---|
| | | | time |
| Starting Material | Product | M + 1 | (min) |
| 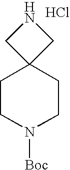 | 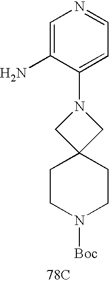 | 319.2 | 0.95 |
| 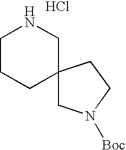 | 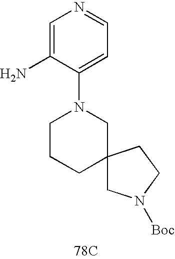 | 333.2 | 1.11 |
| 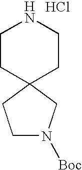 | 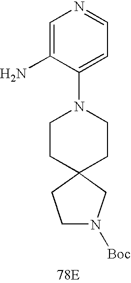 | 333.3 | 1.21 |
| 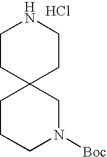 | 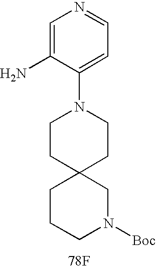 | 347.2 | 1.12 |
| 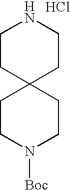 | 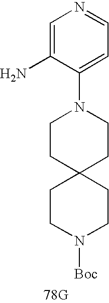 | 347.2 | 1.05 |
| 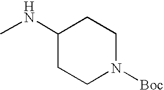 | 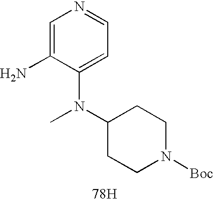 | 307.3 | 0.99 |

### Example 79

### Preparation of Compound 79A

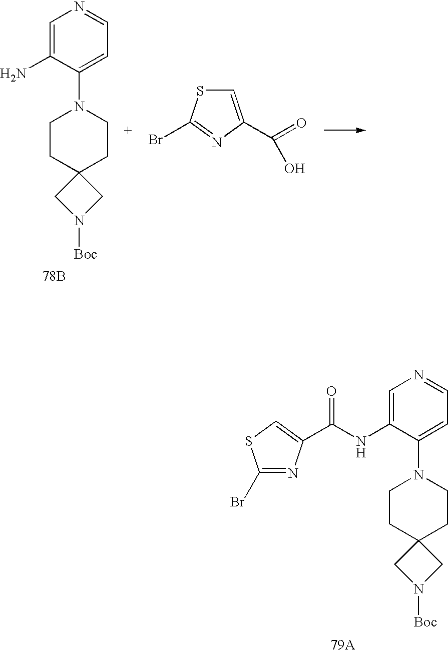

To a solution of 2-bromo thiazole-5 carboxylic acid (0.57 mmol) and HATU (0.68 mmol) in 1 mL of DMF, was added DIPEA (3 equivalents, 1.6 mmol) and the resulting reaction was stirred for 10 minutes at room temperature. To the reaction mixture was then added a solution of compound 78B (0.57 mmol) in 0.5 mL of DMF and the resulting reaction was stirred at room temperature for an additional 2 hours. The reaction mixture was then concentrated in vacuo and the residue obtained was purified using preparative liquid chromatography (5-10% methanol in dichloromethane) to provide 0.54 mmol (95%) of compound 79A.

### Example 80

### Preparation of Compound 221

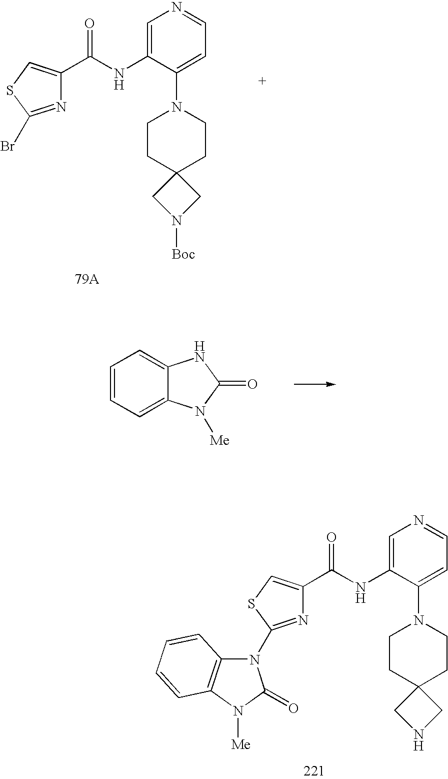

Compound 79A (0.15 mmol), 1-methyl-1,3-dihydro-benzimidazole-2-one (0.1 mmol), Pd₂ dba₃ (0.01 mmol), Xantphos (0.02 mmol), and K₃PO₄ (0.3 mmol) were placed in vial and diluted with dioxane (1 mL). The resulting solution was degassed and flushed with argon, then capped, and sonicated. The reaction was heated to 90° C. and allowed to stir at this temperature for 2 hours, then the reaction mixture was cooled to room temperature and diluted with ethyl acetate. The organic layer was sequentially washed with saturated NaHCO₃ (aq), brine, and water. The organic layer was then dried with sodium sulfate, filtered and concentrated in vacuo and the crude residue obtained was purified using preparative liquid chromatography (5-10% methanol in dichloromethane). The product obtained was then lyophilized and the solid material obtained was treated with excess 2M HCl in dioxane to provide compound 221.

Using the method described above, the following illustrative compounds were made:

| | | | Retention |
|---|---|---|---|
| | | | time |
| Compound | M+(m/z) | M + H | (minutes) |
| 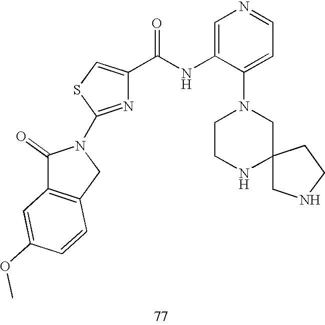 | 504.20 | 505.20 | 2.75 |
| 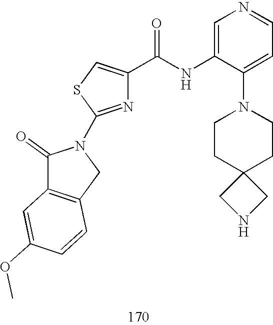 | 490.18 | 491.2 | 2.60 |
| 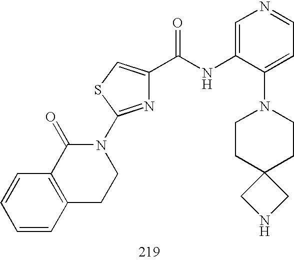 | 474.18 | 475.2 | 2.19 |
| 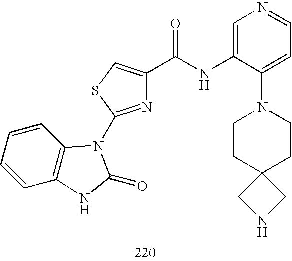 | 461.16 | 462.1 | 1.84 |
| 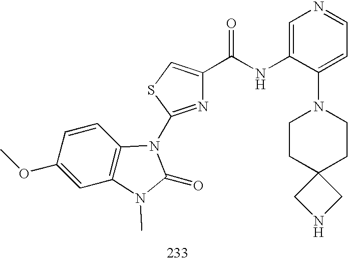 | 505.19 | 506.1 | 2.26 |
| 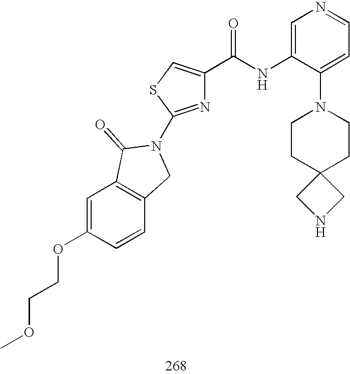 | 534.21 | 535.2 | 2.66 |
| 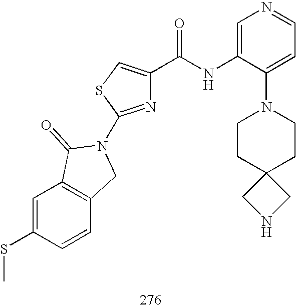 | 506.16 | 507.1 | 2.48 |
| 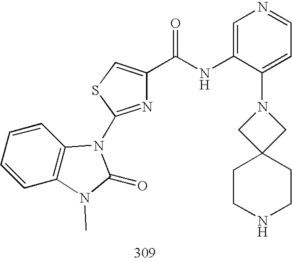 | 475.18 | 476.1 | 1.75 |
| 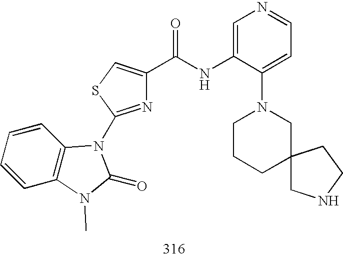 | 489.19 | 490.1 | 2.31 |
| 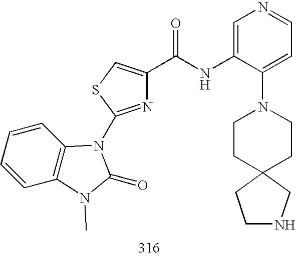 | 489.19 | 490.1 | 2.39 |
| 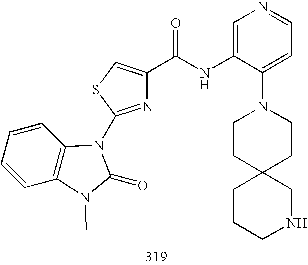 | 503.21 | 504.3 | 2.18 |
| 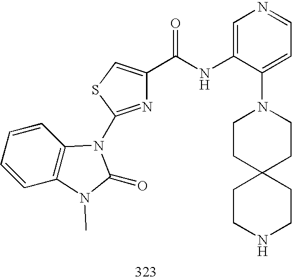 | 503.21 | 504.3 | 2.15 |

### Example 81

### Preparation of Compound 81B

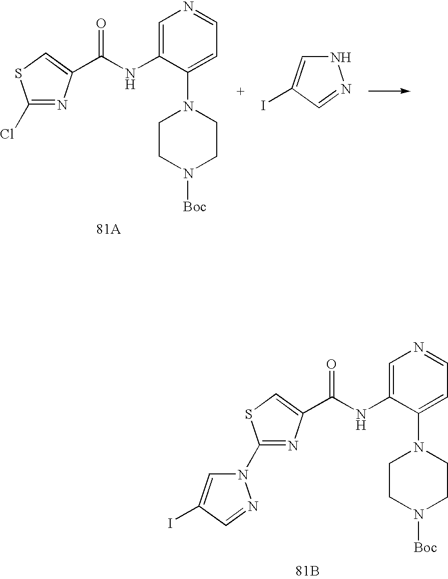

Using the method described in Example 24, compound 81A was reacted with 4-iodopyrazole to prepare compound 81B. Mass calculated for formula C₂₁H₂₄N₇O₂SI 581.0, observed LCMS m/z 582.20 (M+H).

### Example 82

### Preparation of Compounds 62, 63, 66, 70, 272, 277 and 283

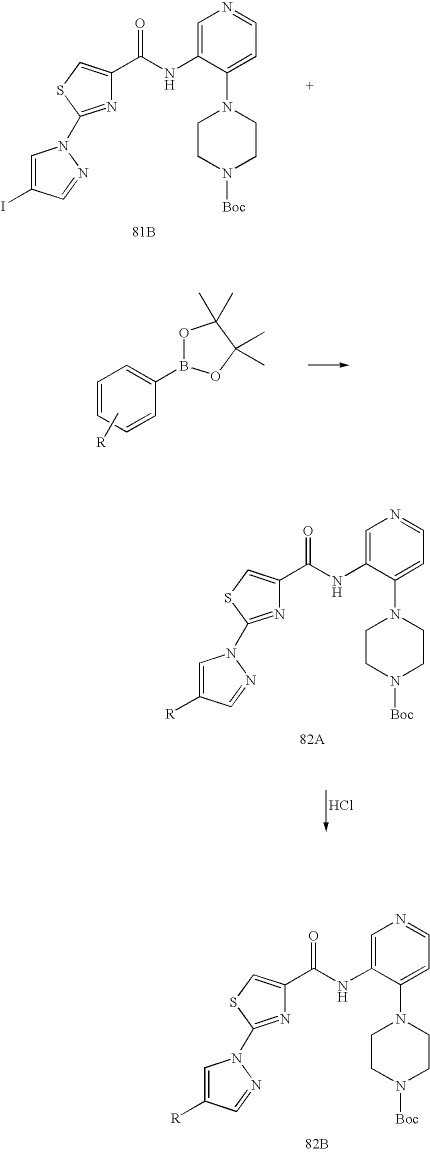

### General Procedure:

Compound 81B (0.30 mmol), a representative boronic acid or ester (0.34 mmol), Pd(dppf)Cl₂ (0.034 mmol), and K₃PO₄ (0.9 mmol) are dissolved in 2 mL of dioxane and 300 □L of water. The resulting solution is degassed and flushed with argon, then heated to 90° C. and allowed to stir at this temperature for about 2 hours. The reaction is then diluted with ethyl acetate and the organic phase is washed sequentially with saturated aqueous NaHCO₃ and water. The organic layer is then dried over Na₂SO₄, filtered and concentrated in vacuo. The residue obtained can be purified using preparative HPLC to provide a compound of formula 82A, which is subsequently lyophilized and the purified product is then treated with 4N HCl to remove the Boc group and afforded the desired product 82B.

Using this method, the following illustrative compounds were made.

| | M + H | Ret time |
|---|---|---|
| Compound | Observed | (min) |
| 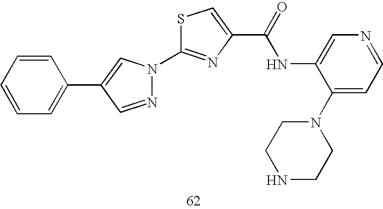 | 432.15 | 2.60 |
| 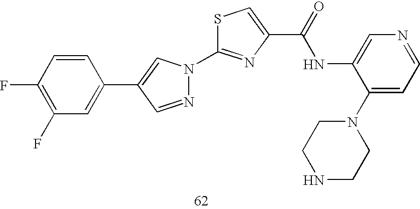 | 468.1 | 2.49 |
| 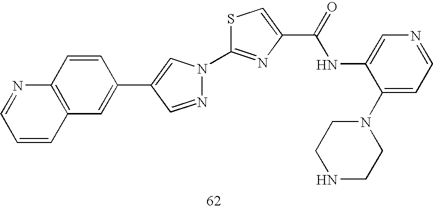 | 483.1 | 1.69 |
| 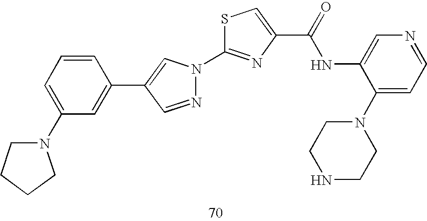 | 501.2 | 2.63 |
| 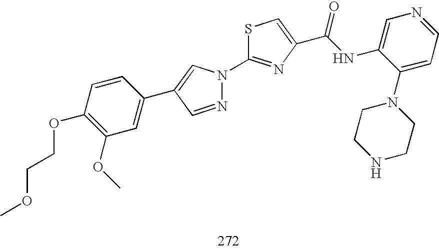 | 536.2 | 2.86 |
| 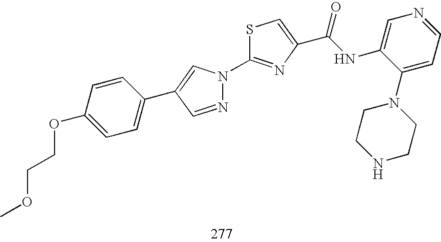 | 506.2 | 2.96 |
| 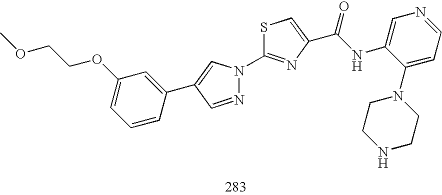 | 506.2 | 2.95 |

### Example 83

### Preparation of Compound 128

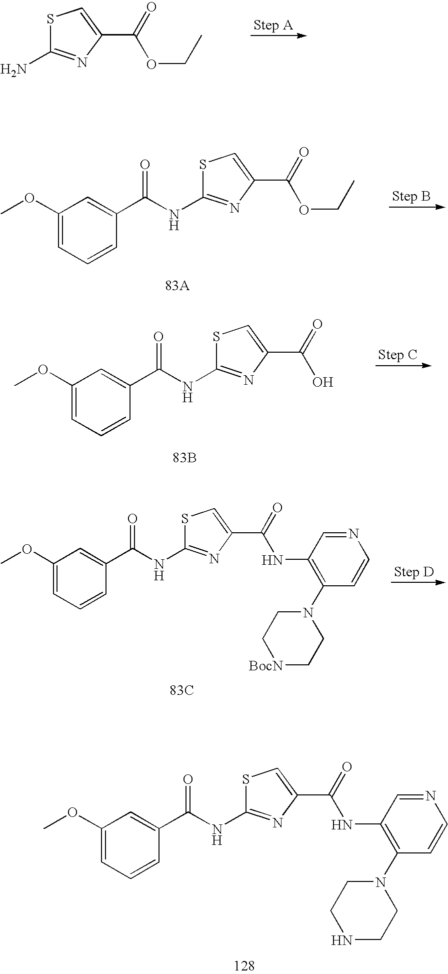

### Step A:

To the mixture of Methyl-2-amino-4-carboxylate (634 mg, 3.69 mmol), DIEA (0.7 mL, 4.0 mmol) and 3-methoxybenzoic acid (561 mg, 3.69 mmol) in DMF (10 mL) was added HATU (1.52 g, 4.0 mmol). The resulting mixture was stirred at room temperature for about 15 hours and water (60 mL) was added. The solid precipitated out was collected with filtration and washed with water, dried under air. The crude product 83A was used in the next step directly without any further purification. HPLC-MS t_{R}=1.71 min (UV_{254 nm}); mass calculated for formula C₁₄H₁₄N₂O₄S 306.1, observed LCMS m/z 307.1 (M+H).

### Step B:

The compound 83B was prepared by hydrolyzing compound 83A using the method described in Example 50 above. HPLC-MS t_{R}=1.45 min (UV_{254 nm}); mass calculated for formula C₁₂H₁₀N₂O₄S 278.0, observed LCMS m/z 279.1 (M+H).

### Step C:

Compound 83C was prepared by reacting compound 83B with the appropriate coupling partner according to the method described in Example 58 above. HPLC-MS t_{R}=1.50 min (UV_{254 nm}); mass calculated for formula C₂₆H₃₀N₆O₅S 538.2, observed LCMS m/z 539.2 (M+H).

### Step D:

Compound 128 was prepared by deprotecting compound 83C using the method described in Example 62 above. HPLC-MS t_{R}=0.94 min (UV_{254 nm}); mass calculated for formula C₂₁H₂₂N₆O₃S 438.1, observed LCMS m/z 439.1 (M+H).

The synthesis of compounds (k) from U.S. provisional patent application, Docket No. OC06760L01 filed of even date herewith, is described below:

### Example 1

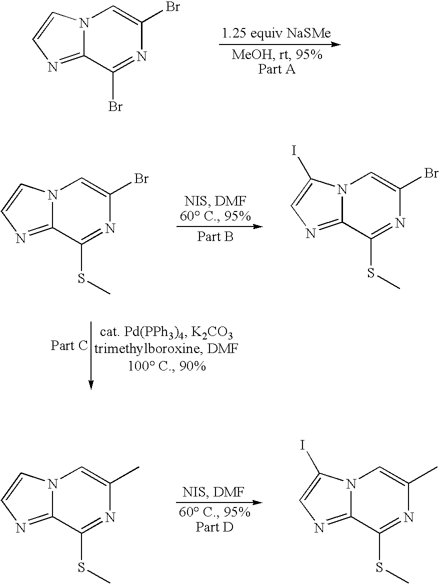

Part A: The title compound was prepared according to US20060106023 (A1).

Part B: To a solution of compound from Example 1, Part A (2.00 g, 8.19 mmol) in DMF (50 mL) was added N-iodosuccinimide (1.84 g, 8.19 mmol). The reaction mixture was stirred at 60 C for 16 hours. The mixture was cooled to 25 C and concentrated. The residue was dissolved in DCM with a small amount of methanol and then loaded on the column. Purification by column chromatography (SiO₂, 40% ethyl acetate/hexanes) afforded compound as a white solid 2.30 g (76%). ¹H-NMR (400 MHz, DMSO-d₆) δ 8.3 (s, 1H), 7.8 (s, 1H), 2.6 (s, 3H). HPLC-MS t_{R}=1.87 Min (UV₂₅₄ₙₘ). Mass calculated for formula C₇H₅BrIN3S 370.01, observed LC/MS m/z 370.9 (M+H).

Part C: A suspension of bromide from Part A (45.6 g), Pd(PPh3)4 (10.8 g), potassium carbonate (77.4 g), trimethylboroxine (46.9 g) and potassium carbonate (77.4 g) in DMF (410 mL) was heated overnight under nitrogen at 105 C. After cooling, the mixture was diluted with ethyl acetate (1 L), washed with brine (2×500 mL), dried (magnesium sulfate), filtered, concentrated and purified by chromatography on silica gel. The title compound was obtained as a pale yellow solid (21.4 g, 64%).

Part D: To a DMF (400 mL) solution of compound from Example 1, Part C (21.8 g) was added N-iodosuccinimide (26.9 g) and the resulting mixture was heated overnight at 60 C. The mixture was concentrated and water (400 mL) was added. After stirring 1 hr at rt, saturated sodium carbonate was added (250 mL) and subsequently stirred an additional 30 min at rt. The mixture was filtered, washed with water, methanol (100 mL) and the filter cake was dried overnight under vacuum. A brown solid was obtained (31.4 g, 87%).

### Example 2

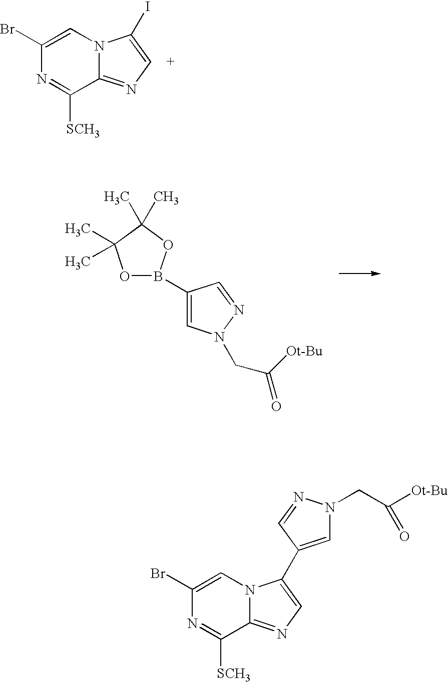

A solution of tert-butyl 2-(4-4,4,5,5-tetramethyl-1,3,2-dioxaborolan-2-yl)-1H-pyrazol-1-yl)acetate (318 mg, 1.03 mmol) in 1,4-dioxane (3 mL) and water (0.30 mL) was added to an argon degassed mixture of compound from Example 1, Part B (292 mg, 0.79 mmol), Pd(dppf)Cl₂ (58 mg, 0.079 mmol) and potassium phosphate (503 mg, 2.37 mmol). The reaction was heated to 40° C. and allowed to stir for 12 hours. The reaction was cooled to room temperature, filtered through Celite eluting with ethyl acetate then concentrated to dryness. Purification of the crude residue by flash chromatography (SiO₂, 12 g; 5% to 40% ethyl acetate in hexanes) afforded the title compound as a light brown solid 244 mg (73%). ¹H NMR (300 MHz, CDCl₃) δ 7.94 (s, 1H), 7.81 (s, 1H), 7.80 (s, 1H), 7.65 (s, 1H), 4.94 (s, 2H), 2.68 (s, 3H), 1.51 (s, 9H).

### Example 3

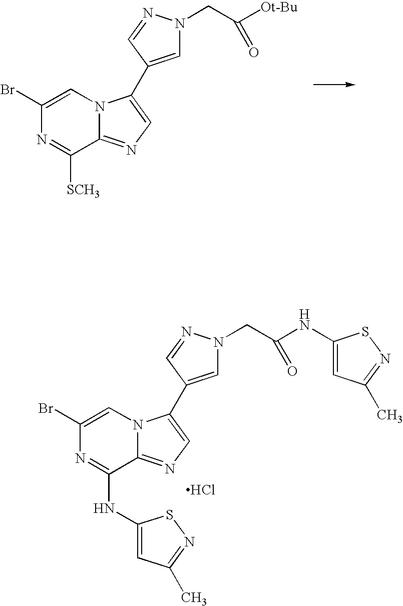

3-Chloroperoxybenzoic acid (204 mg, 1.18 mmol) was added to a room temperature solution of ester from Example 2 (244 mg, 0.58 mmol) in methylene chloride (3 mL). The reaction was allowed to stir for 1 hour. Upon completion, the reaction was concentrated to dryness then taken up into ethyl acetate (50 mL). The solution was washed with saturated aqueous sodium bicarbonate solution (50 mL) and brine (2×50 mL) then dried (sodium sulfate), filtered and concentrated to dryness to give 220 mg of crude sulfoxide. The crude sulfoxide (220 mg, 0.48 mmol) in dimethylsulfoxide (2.5 mL) was added to a premixed solution of sodium hydride (106 mg, 1.45 mmol) and 2-amino-4-methylisothiazole (78 mg, 0.68 mmol) in dimethylsulfoxide (2.5 mL). The reaction was stirred for 20 minutes then quenched with saturated aqueous ammonium chloride (50 mL). The aqueous layer was extracted with diethyl ether (2×50 mL) and ethyl acetate (2×50 mL). The combined organic layers were washed with brine (2×50 mL), dried (sodium sulfate), filtered and concentrated to dryness. Purification of the resultant residue by flash chromatography (SiO₂, 12 g; 5% to 40% ethyl acetate in methylene chloride) and then on prep-HPLC afforded the title compound as a yellow solid 2 mg (0.8%). ¹H NMR (300 MHz, CDCl₃) δ 8.35 (s, 1H), 8.32 (s, 1H), 8.02 (s, 1H), 7.93 (s, 1H), 7.17 (s, 1H), 6.90 (s, 1H), 5.37 (s, 2H), 2.63 (s, 3H), 2.46 (s, 3H). HPLC t_{R}=4.62 min (UV₂₅₄ₙₘ). Mass calculated for formula C₁₉H₁₆BrN₉OS₂ 529.01; observed MH⁺ (APCI MS) 531.1 (m/z).

### Example 4

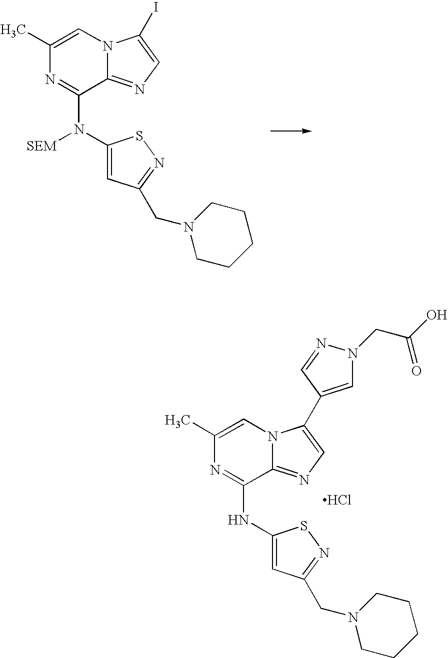

A solution of tert-butyl 2-(4-(4,4,5,5-tetramethyl-1,3,2-dioxaborolan-2-yl)-1H-pyrazol-1-yl)acetate (135 mg, 0.44 mmol) in 1,4-dioxane (3 mL) and water (0.30 mL) was added to a nitrogen flushed mixture of iodide (200 mg, 0.34 mmol), Pd(dppf)Cl₂ (25 mg, 0.034 mmol) and potassium phosphate (216 mg, 1.02 mmol). The reaction mixture was heated to 90° C. and allowed to stir for 12 hours. Upon completion, the reaction was allowed to cool to room temperature and then was concentrated to dryness. Purification of the resultant residue by flash chromatography (SiO₂; 12 g; 10% to 80% ethyl acetate in methylene chloride) afforded the desired coupled intermediate. Trifluoroacetic acid (1 mL) was added to a room temperature solution of the desired coupled ester (80 mg, 0.125 mmol) in methylene chloride (3 mL). The reaction was stirred for 12 hours then concentrated to dryness. Purification of the resultant residue by prep-HPLC afforded the title compound as a yellow solid 40 mg (45%). ¹H NMR (300 MHz, CD₃OD) δ 8.34 (s, 1H), 8.26 (s, 1H), 8.08 (s, 1H), 8.01 (s, 1H), 7.36 (s, 1H), 5.15 (s, 2H), 4.43 (s, 2H), 3.17-2.99 (m, 2H), 2.71-2.55 (m, 2H), 2.64 (s, 3H), 2.06-1.71 (m, 5H), 1.65-1.46 (m, 1H). HPLC t_{R}=3.82 min (UV₂₅₄ₙₘ). Mass calculated for formula C₂₁H₂₄N₈O₂S 452.17; observed MH⁺ (ESI MS) 453.1 (m/z).

### Example 5

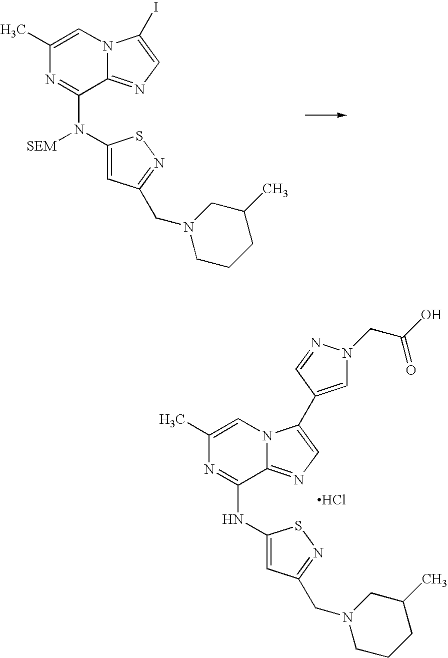

Example 5 was prepared in a similar manner to Example 4. ¹H NMR (300 MHz, CD₃OD) δ 8.34 (s, 1H), 8.26 (s, 1H), 8.08 (s, 1H), 8.01 (s, 1H), 7.36 (s, 1H), 5.15 (s, 2H), 4.43 (s, 2H), 3.17-2.99 (m, 2H), 2.71-2.55 (m, 2H), 2.64 (s, 3H), 2.06-1.71 (m, 5H), 1.65-1.46 (m, 1H). HPLC t_{R}=3.82 min (UV₂₅₄ₙₘ). Mass calculated for formula C₂₁H₂₄N₈O₂S 452.17; observed MH⁺ (ESI MS) 453.1 (m/z).

### Example 6

The compounds shown in column 2 of Table 1 (shown immediately below) were prepared as follows:

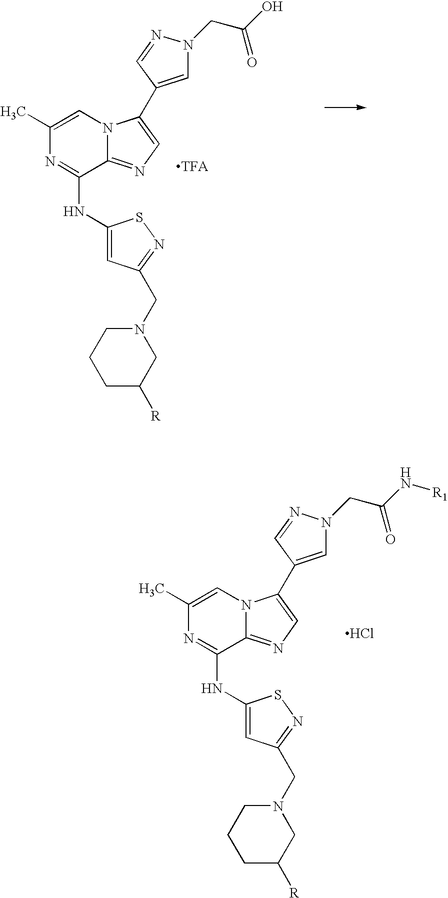

A mixture of acid (1 equivalent), the respective amine (1.5 equivalents), HATU (1.5 equivalents), and diisopropylethylamine (3 equivalents) in anhydrous DMF (500 μL) was stirred at room temperature for 2 hours. The reaction was then concentrated under reduced pressure, purified by preparative HPLC and conversion to the hydrochloride salt afforded compounds shown in Column 2 of Table 1 (shown immediately below):

TABLE 1

| | | | MS | |
|---|---|---|---|---|
| | | | MH⁺ | HPLC |
| Example | Column 2 | MW | m/z | t_{R} |
| 6-1 | 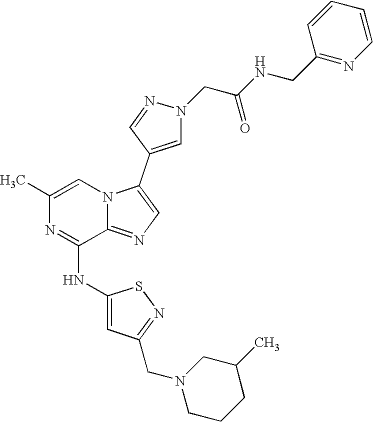 | 556.2 | 557.4 | 3.51 |
| 6-2 | 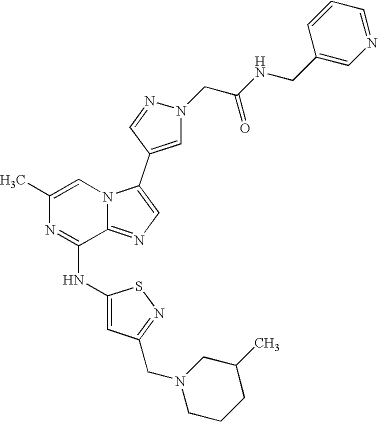 | 556.2 | 557.2 | 3.48 |
| 6-3 | 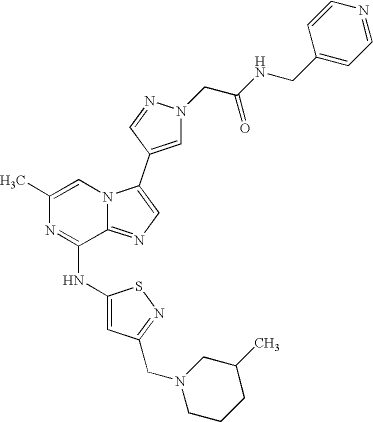 | 556.2 | 557.3 | 3.48 |
| 6-4 | 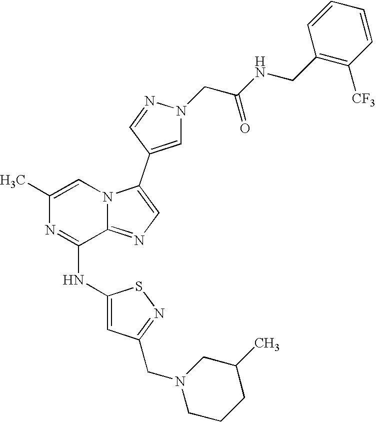 | 623.2 | 624.6 | 5.34 |
| 6-5 | 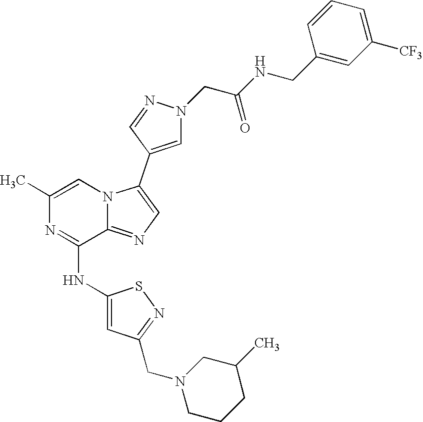 | 623.2 | 624.6 | 5.35 |
| 6-6 | 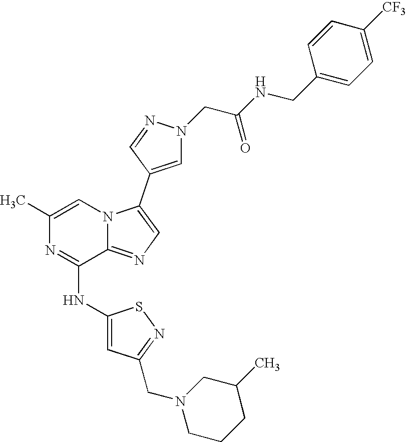 | 623.2 | 624.5 | 5.39 |
| 6-7 | 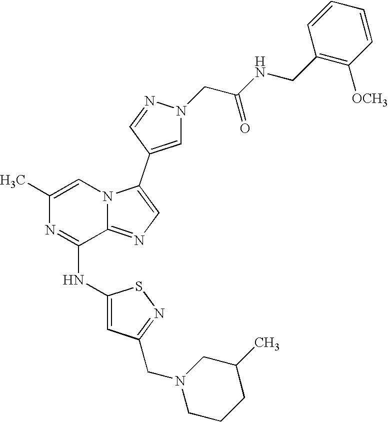 | 585.2 | 586.9 | 4.96 |
| 6-8 | 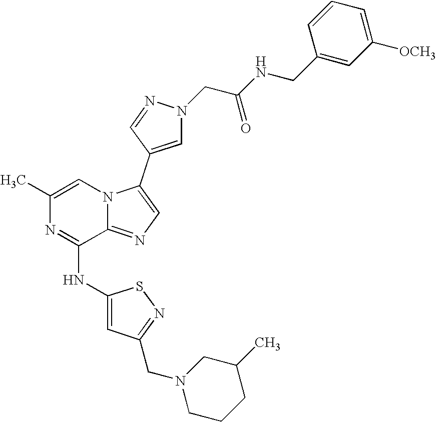 | 585.2 | 586.3 | 4.85 |
| 6-9 | 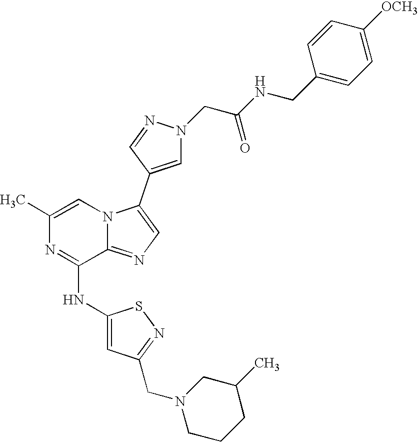 | 585.2 | 586.2 | 4.82 |
| 6-10 | 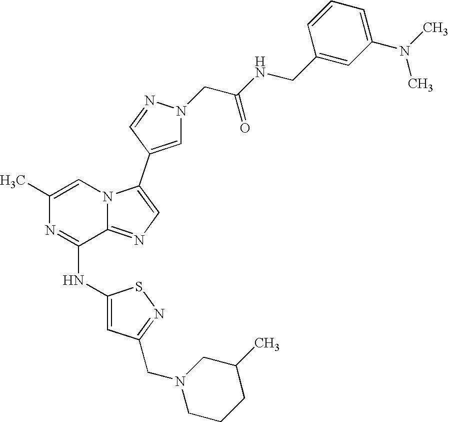 | 598.3 | 599.8 | 3.71 |
| 6-11 | 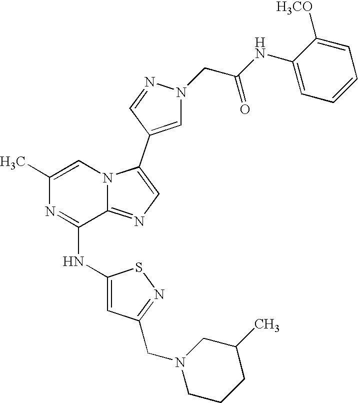 | 571.2 | 572.4 | 5.12 |
| 6-12 | 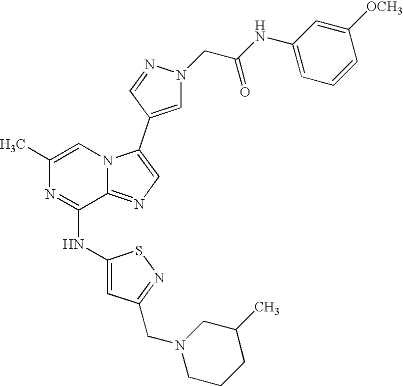 | 571.2 | 572.4 | 4.98 |
| 6-13 | 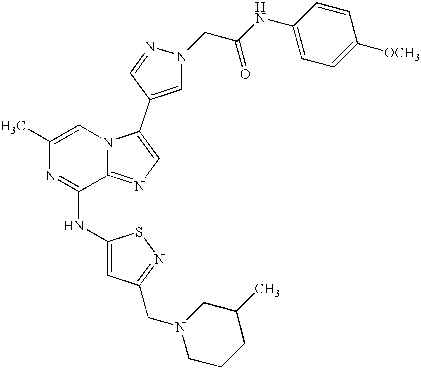 | 571.2 | 572.5 | 4.87 |
| 6-14 | 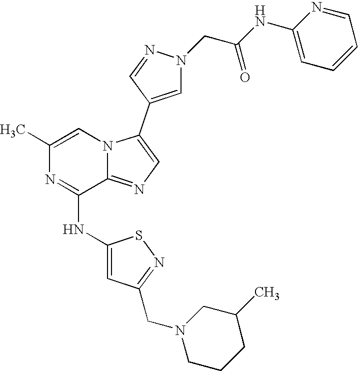 | 542.2 | 543.7 | 3.93 |
| 6-15 | 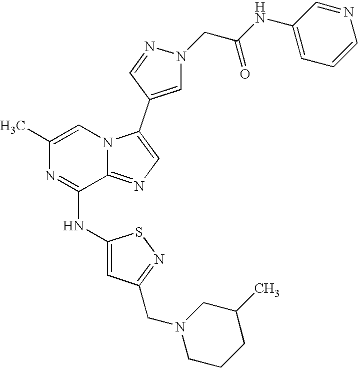 | 542.2 | 543.6 | 3.60 |
| 6-16 | 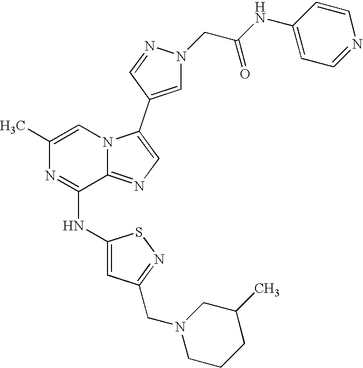 | 542.2 | 543.6 | 3.67 |
| 6-17 | 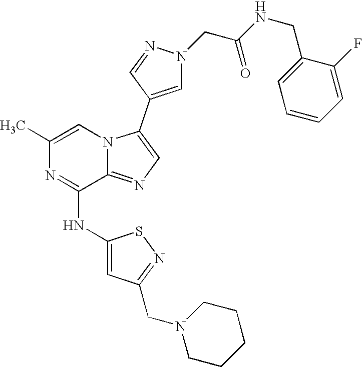 | 559.6 | 260.2 | 5.10 |
| 6-18 | 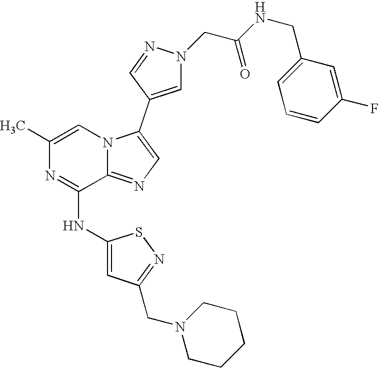 | 559.6 | 560.3 | 5.13 |
| 6-19 | 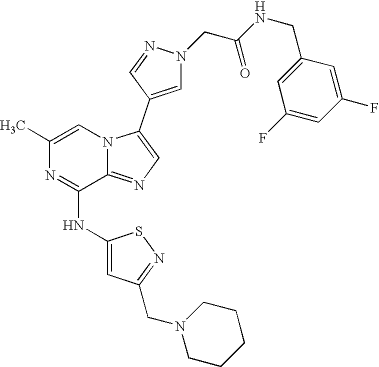 | 577.6 | 578.9 | 5.26 |
| 6-20 | 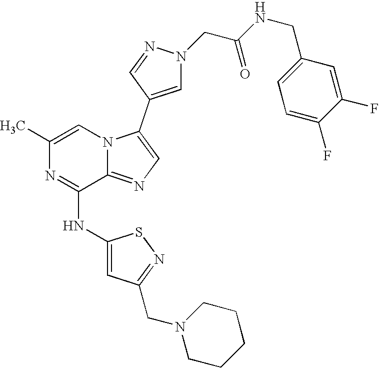 | 563.6 | 564.7 | 5.43 |
| 6-21 | 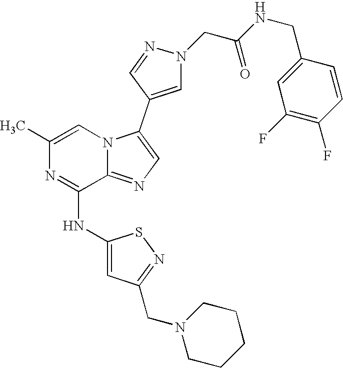 | 563.6 | 564.7 | 5.34 |
| 6-22 | 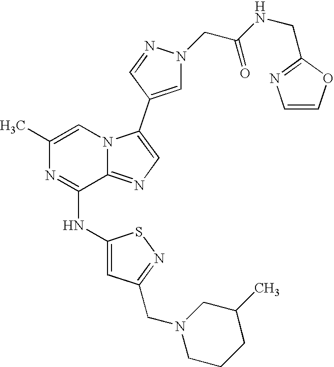 | 546.6 | 547.6 | 3.96 |
| 6-23 | 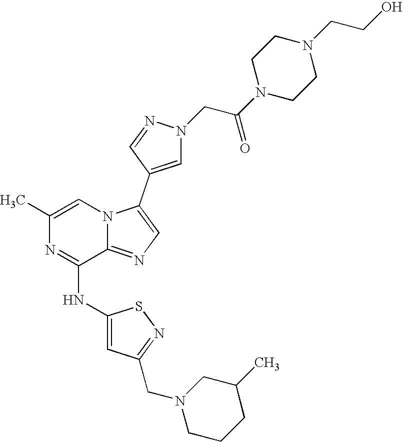 | 578.7 | 579.8 | 3.39 |
| 6-24 | 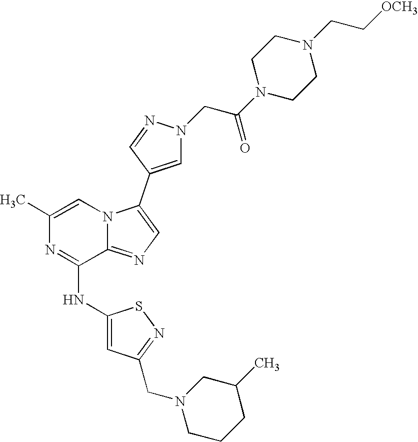 | 592.7 | 593.9 | 3.52 |
| 6-25 | 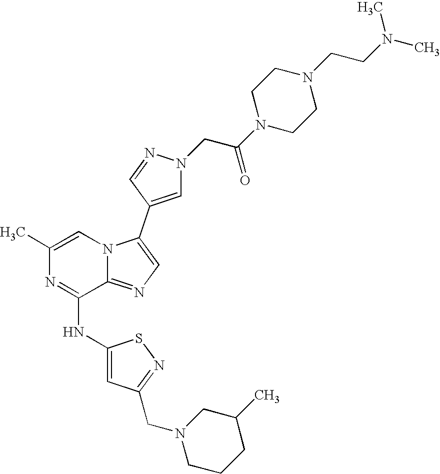 | 605.8 | 606.8 | 3.35 |
| 6-26 | 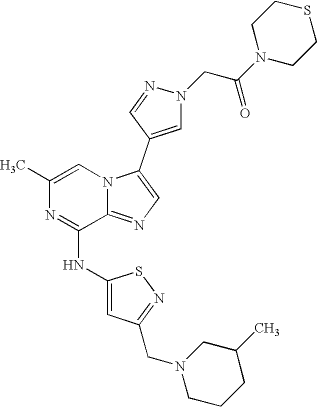 | 551.7 | 552.8 | 4.47 |
| 6-27 | 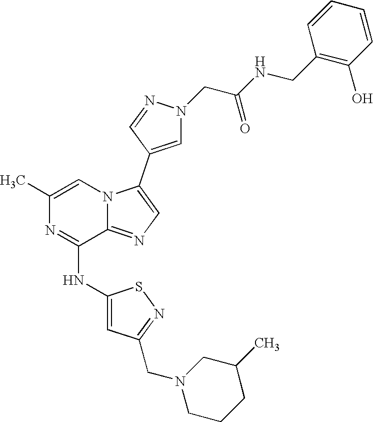 | 571.7 | 572.5 | 4.59 |
| 6-28 | 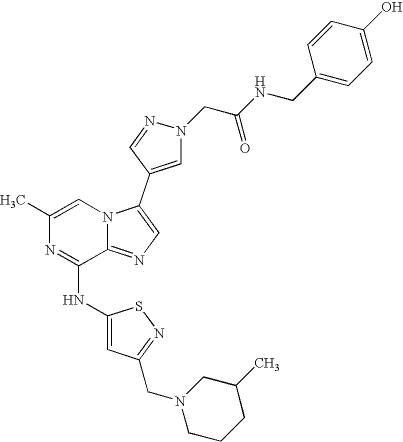 | 571.7 | 572.4 | 4.26 |
| 6-29 | 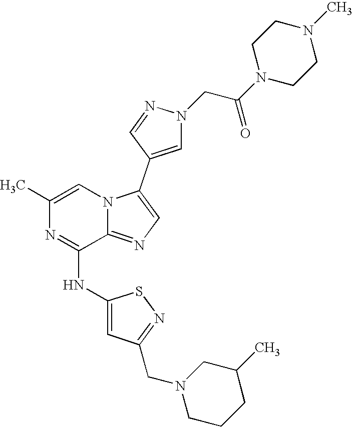 | 548.7 | 549.8 | 3.45 |
| 6-30 | 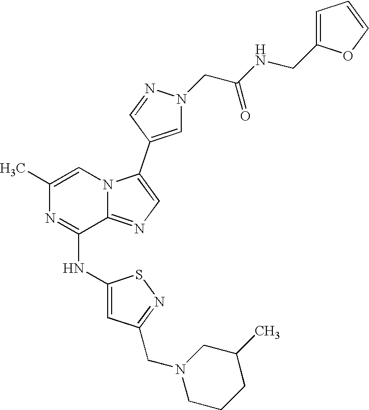 | 545.6 | 546.5 | 4.53 |
| 6-31 | 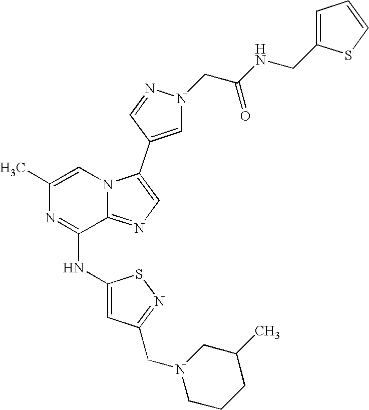 | 561.7 | 562.1 | 4.72 |
| 6-32 | 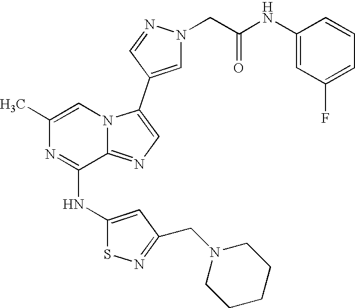 | 545.2 | 546.5 | 5.08 |
| 6-33 | 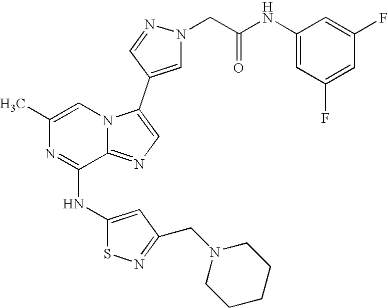 | 563.2 | 564.7 | 5.32 |
| 6-34 | 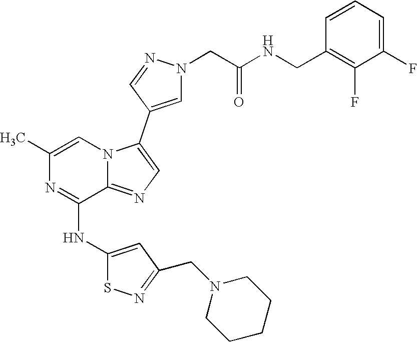 | 577.2 | 578.9 | 4.97 |
| 6-35 | 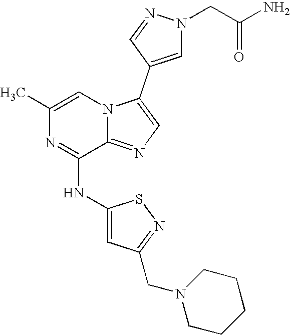 | 451.1 | 452.2 | 3.44 |
| 6-36 | 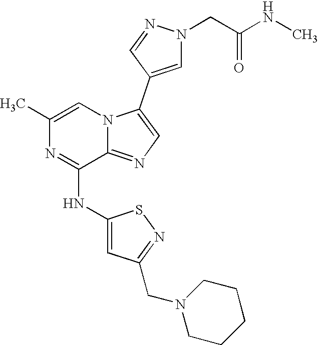 | 465.2 | 466.7 | 3.57 |
| 6-37 | 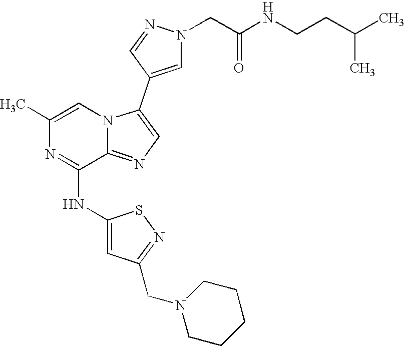 | 521.2 | 522.9 | 4.71 |
| 6-38 | 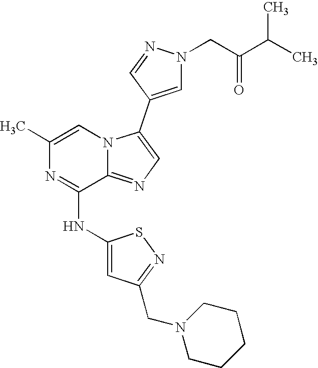 | 479.2 | 480.1 | 3.73 |
| 6-39 | 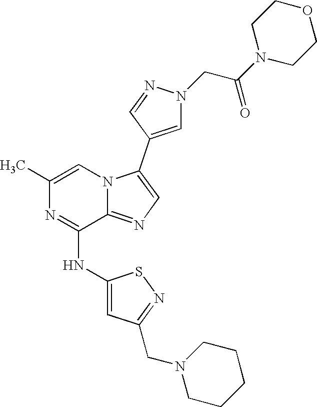 | 521.2 | 522.9 | 3.73 |
| 6-40 | 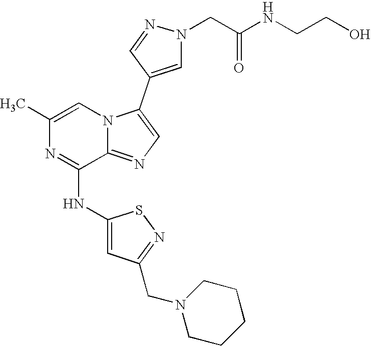 | 495.2 | 496.9 | 3.42 |
| 6-41 | 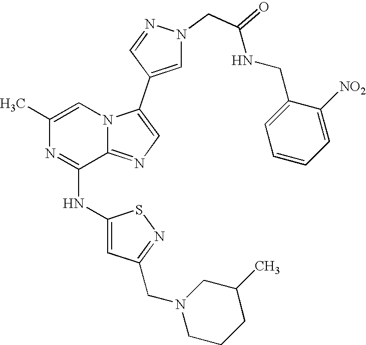 | 600.2 | 601.7 | 4.91 |
| 6-42 | 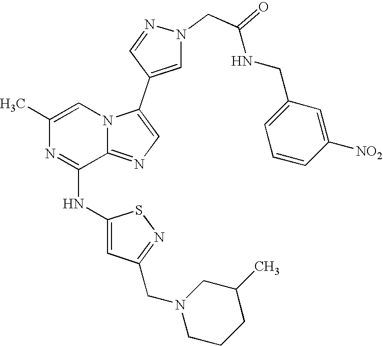 | 600.2 | 601.6 | 4.90 |
| 6-43 | 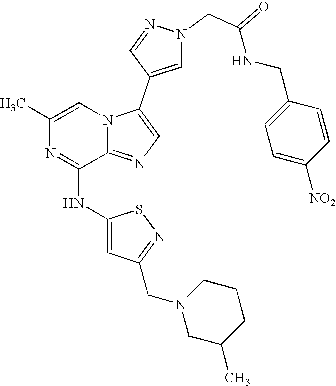 | 600.2 | 601.6 | 4.90 |
| 6-44 | 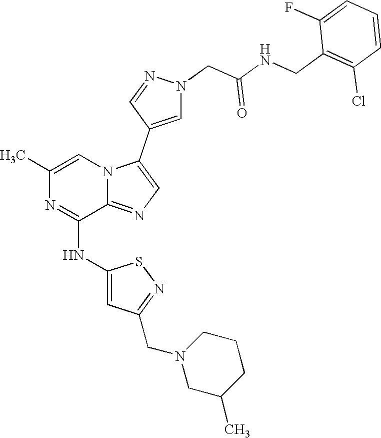 | 607.2 | 608.5 | 5.12 |
| 6-45 | 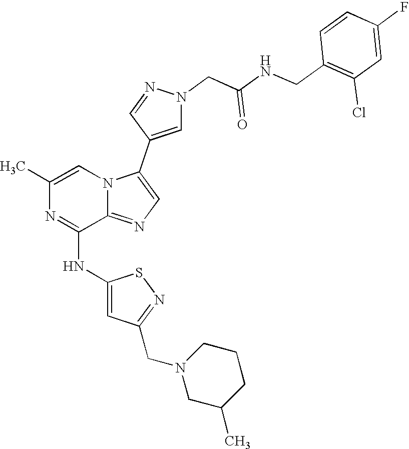 | 607.2 | 608.4 | 5.23 |
| 6-46 | 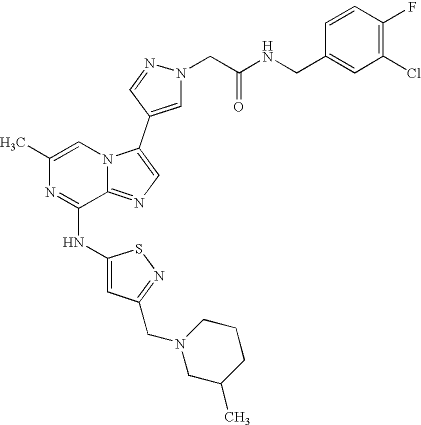 | 607.2 | 609.4 | 5.23 |
| 6-47 | 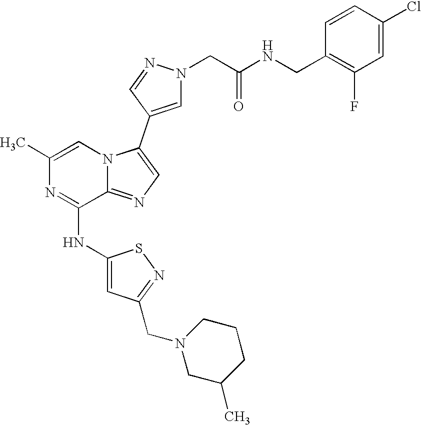 | 607.2 | 609.4 | 5.27 |
| 6-48 | 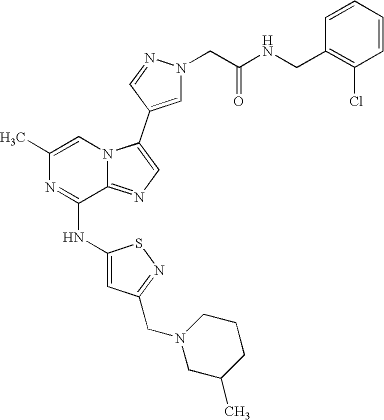 | 589.2 | 591.8 | 5.15 |
| 6-49 | 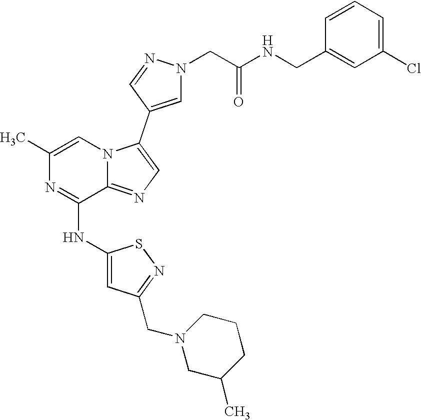 | 589.2 | 591.8 | 5.16 |
| 6-50 | 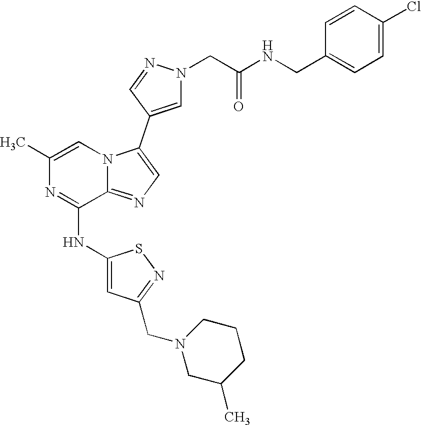 | 589.2 | 591.8 | 5.17 |
| 6-51 | 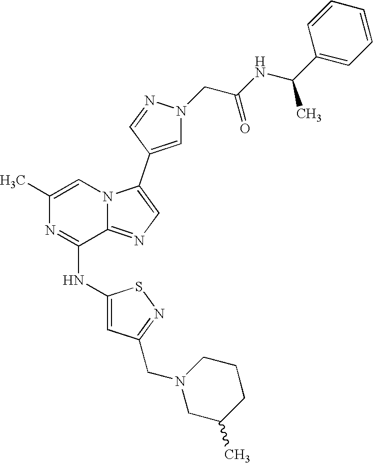 | 569.3 | 570.7 | 5.01 |
| 6-52 | 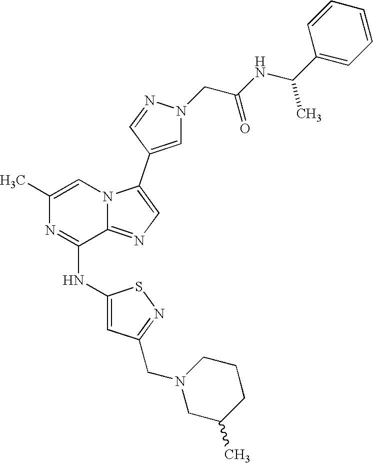 | 569.3 | 570.7 | 4.99 |
| 6-53 | 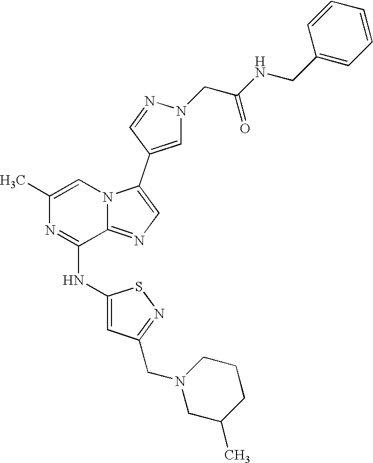 | 555.3 | 556.3 | 4.82 |
| 6-54 | 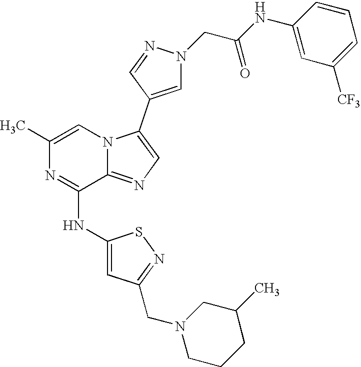 | 609.6 | 610.4 | 5.54 |
| 6-55 | 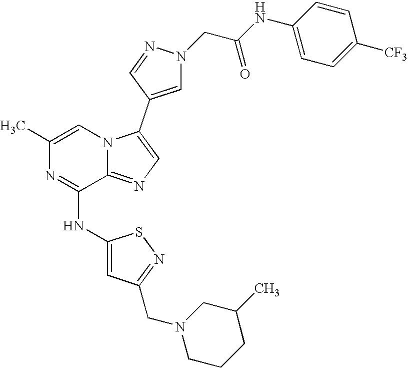 | 609.6 | 610.6 | 5.59 |
| 6-56 | 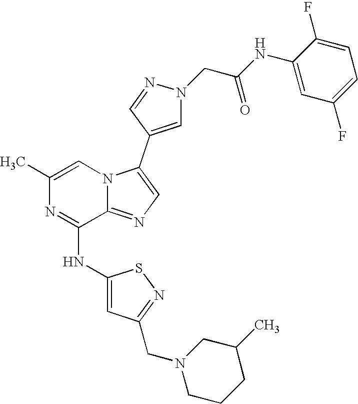 | 577.6 | 578.8 | 5.17 |
| 6-57 | 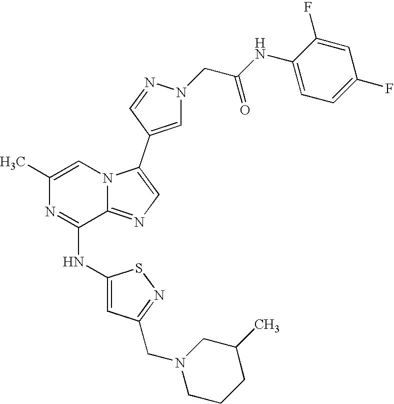 | 577.6 | 578.9 | 5.03 |
| 6-58 | 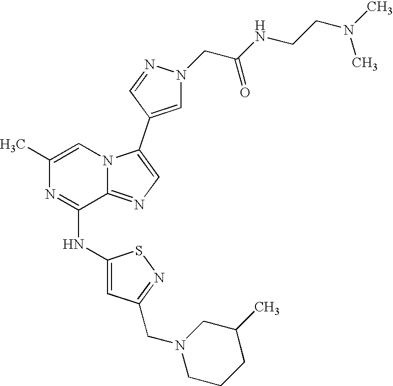 | 536.7 | 537.7 | 3.45 |
| 6-59 | 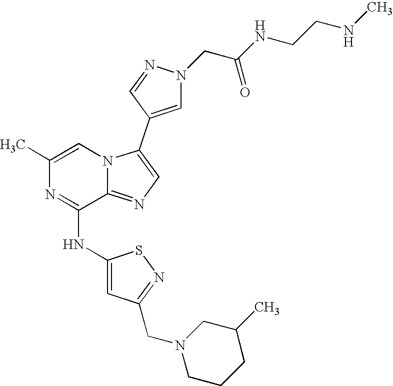 | 522.6 | 523.9 | 3.43 |
| 6-60 | 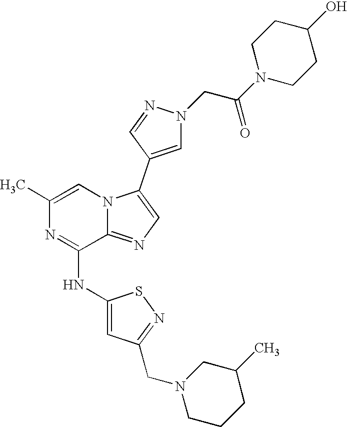 | 549.6 | 550.7 | 3.76 |
| 6-61 | 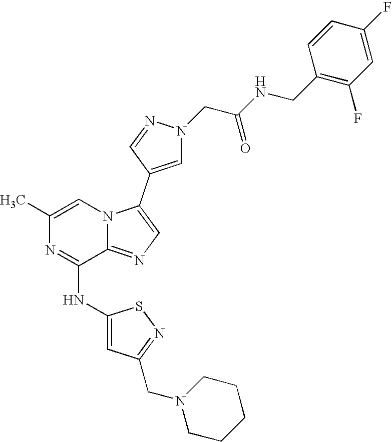 | 577.22 | 578.9 | 5.20 |
| 6-62 | 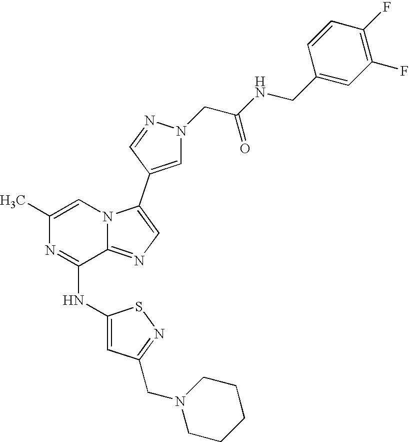 | 577.22 | 578.9 | 5.25 |
| 6-63 | 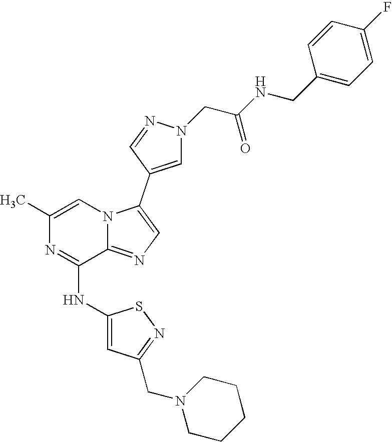 | 559.23 | 560.2 | 5.12 |
| 6-64 | 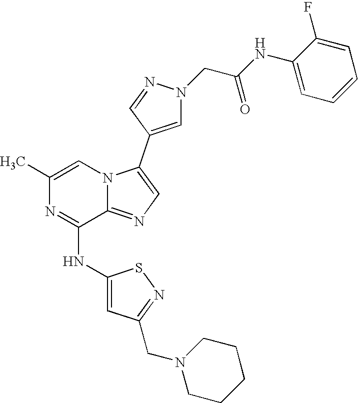 | 545.21 | 546.5 | 5.17 |
| 6-65 | 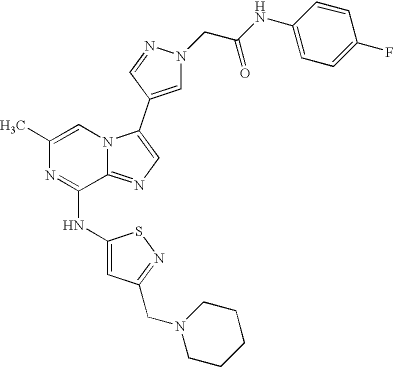 | 545.21 | 546.4 | 5.21 |
| 6-66 | 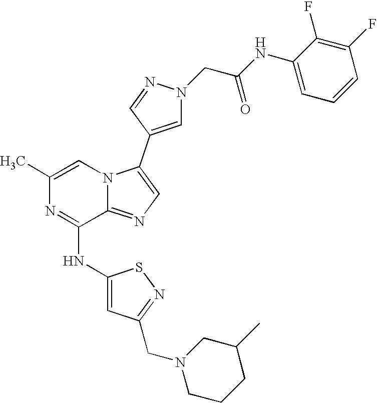 | 577.6 | 578.9 | 5.14 |
| 6-67 | 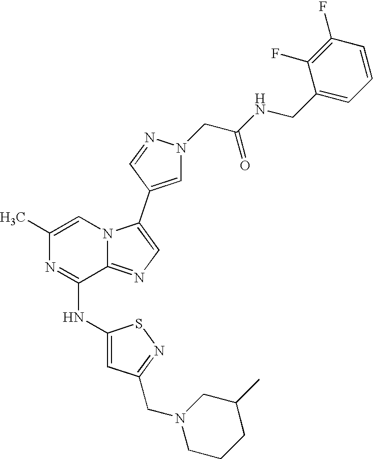 | 591.6 | 592.8 | 5.03 |
| 6-68 | 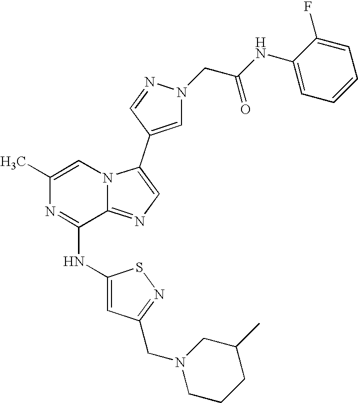 | 559.6 | 560.4 | 5.00 |
| 6-69 | 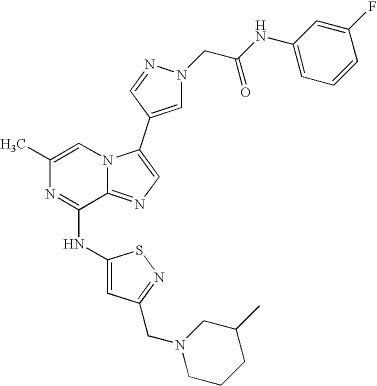 | 559.6 | 560.4 | 5.11 |
| 6-70 | 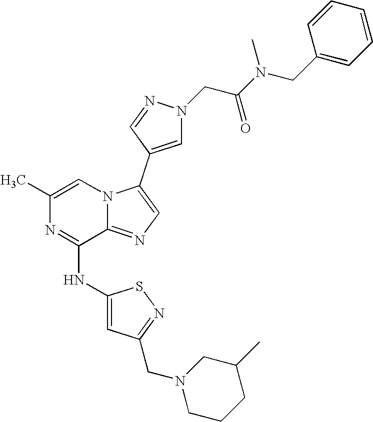 | 569.7 | 570.6 | 5.05 |
| 6-71 | 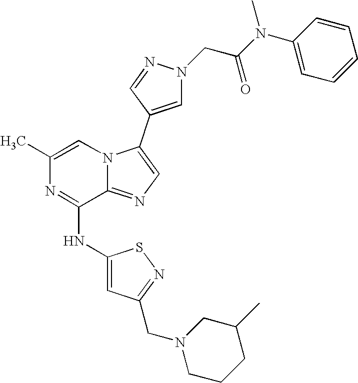 | 555.6 | 556.4 | 4.96 |
| 6-72 | 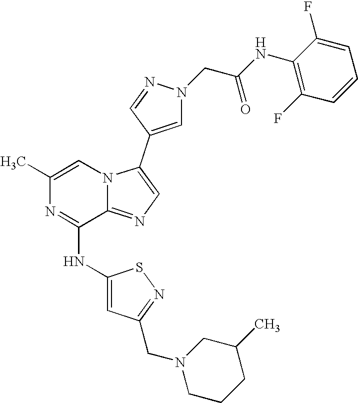 | 577.6 | 578.3 | 4.85 |
| 6-73 | 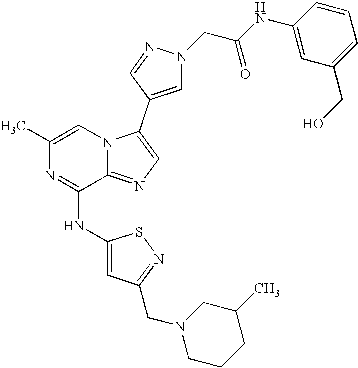 | 571.6 | 572.4 | 4.33 |
| 6-74 | 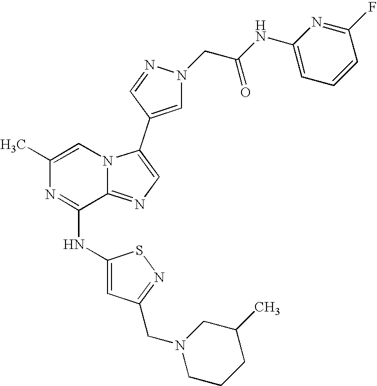 | 560.6 | 561.3 | 4.92 |
| 6-75 | 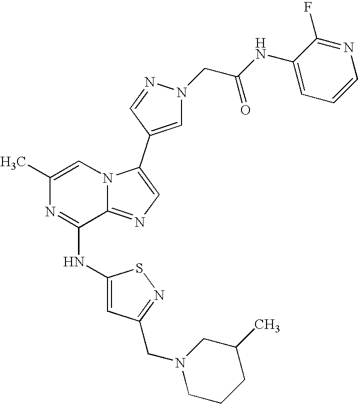 | 560.6 | 561.2 | 4.52 |
| 6-76 | 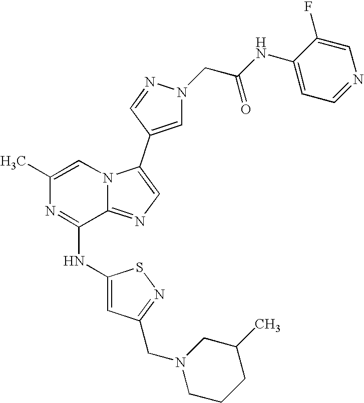 | 560.6 | 561.2 | 3.75 |
| 6-77 | 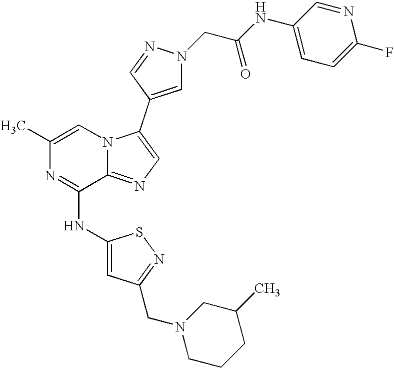 | 560.6 | 561.3 | 4.53 |
| 6-78 | 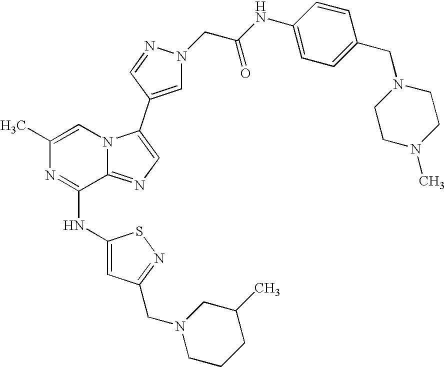 | 653.8 | 654.8 | 3.63 |
| 6-79 | 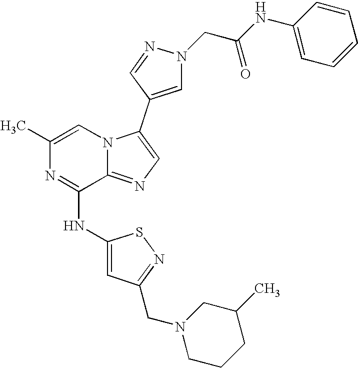 | 541.6 | 542.6 | 4.91 |
| 6-80 | 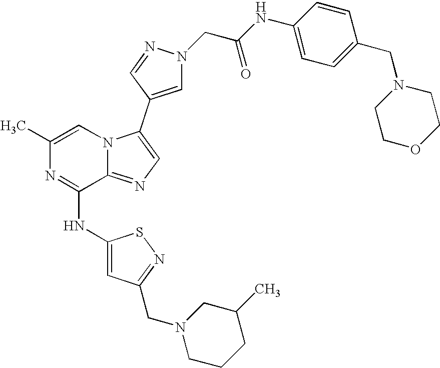 | 640.8 | 641.6 | 3.84 |
| 6-81 | 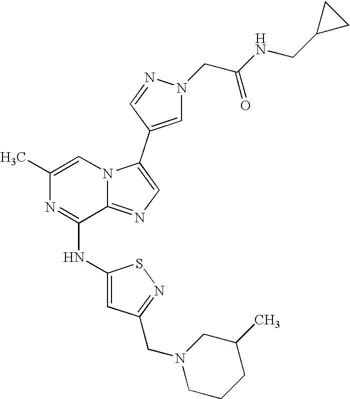 | 519.6 | 520.4 | 4.42 |
| 6-82 | 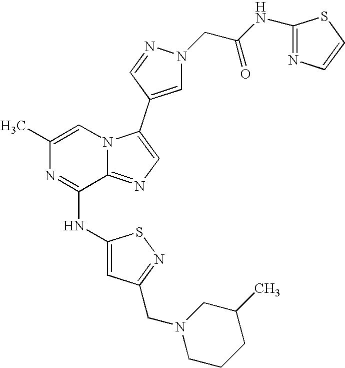 | 548.6 | 549.6 | 4.44 |
| 6-83 | 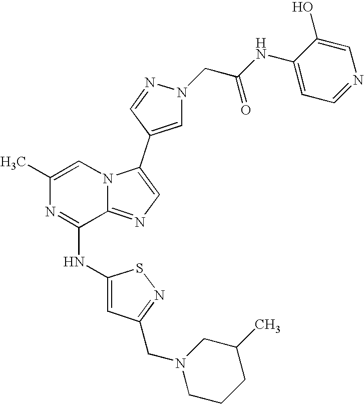 | 558.6 | 559.4 | 3.79 |
| 6-84 | 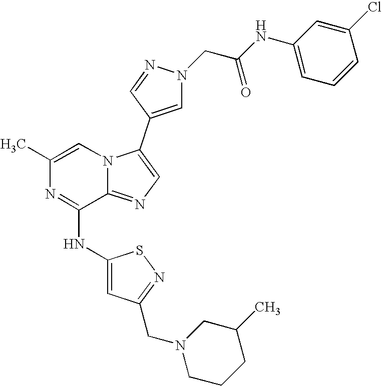 | 576.1 | 576.8 | 5.41 |
| 6-85 | 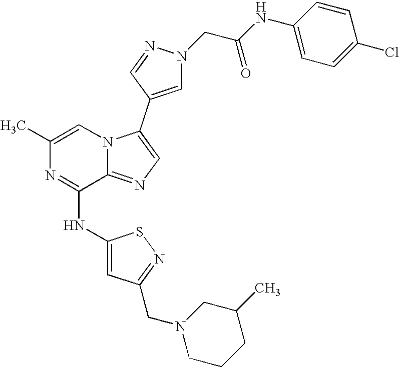 | 576.1 | 576.8 | 5.37 |
| 6-86 | 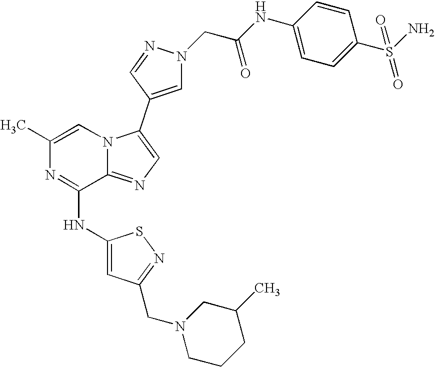 | 620.7 | 621.7 | 4.28 |
| 6-87 | 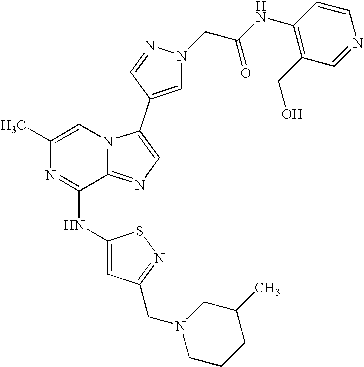 | 572.6 | 573.3 | 3.77 |
| 6-88 | 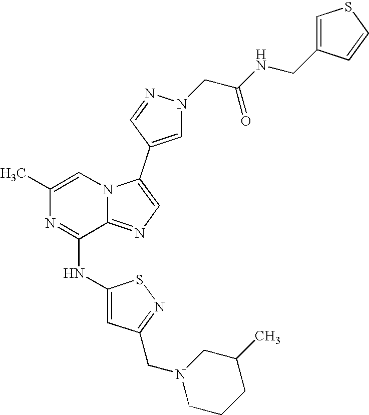 | 561.7 | 562.3 | 4.71 |
| 6-89 | 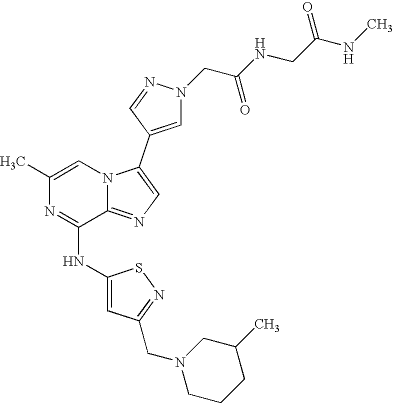 | 536.6 | 537.6 | 3.69 |
| 6-90 | 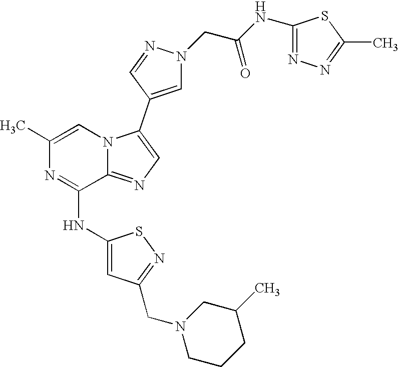 | 563.7 | 564.9 | 4.24 |
| 6-91 | 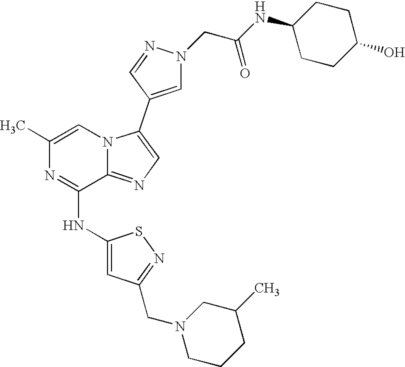 | 563.7 | 564.8 | 3.89 |
| 6-92 | 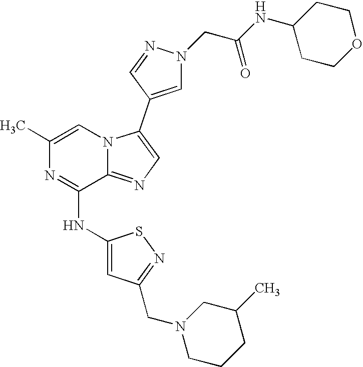 | 549.6 | 550.8 | 3.98 |
| 6-93 | 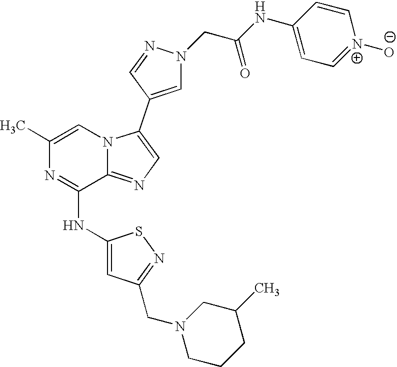 | 558.6 | 559.5 | 3.72 |
| 6-94 | 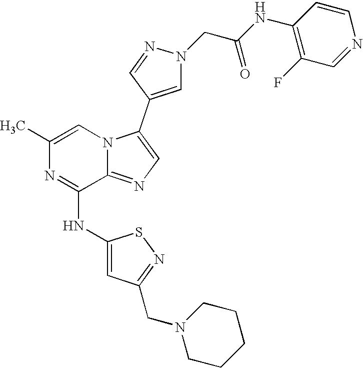 | 546.6 | 547.7 | 3.52 |
| 6-95 | 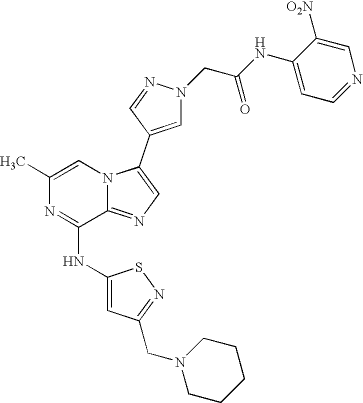 | 573.6 | 574.5 | 4.47 |
| 6-96 | 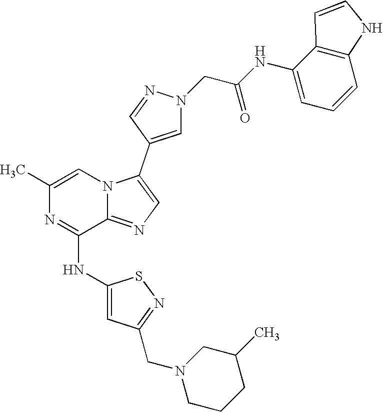 | 566.6 | 567.7 | 4.63 |
| 6-97 | 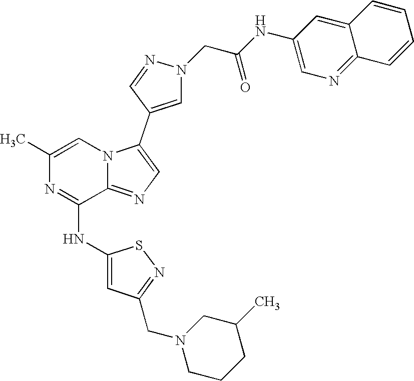 | 592.7 | 593.9 | 4.16 |
| 6-98 | 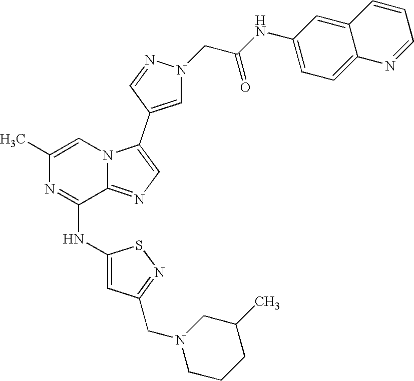 | 592.7 | 593.9 | 3.84 |

### Example 7

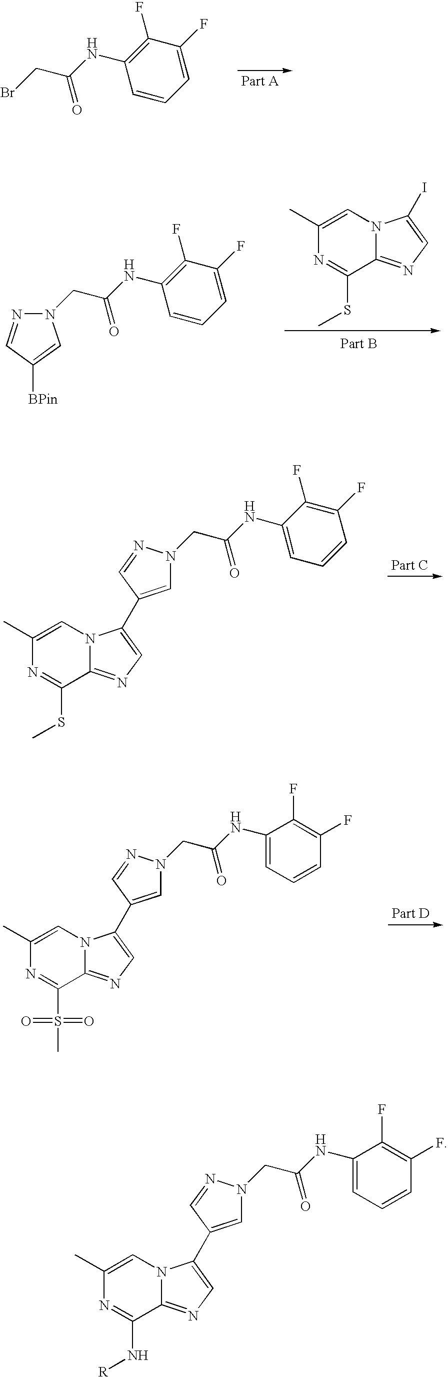

Part A: To a solution of 4-(4,4,5,5-Tetramethyl-1,3,2-dioxaborolan-2-yl)-1H-pyrazole (2.2 g, 11.32) and bromide (2.83 g, 11.32 mmol) in DMA (5 mL) was added potassium carbonate (1.9 g, 13.6 mmol). The mixture was heated at 60 C for 20 hr. To the reaction mixture was added half sat'd ammonium chloride and ethyl acetate. The organic phase was washed with water (2×), brine and dried (sodium sulfate). Concentration and purification by chromatography (50% ethyl acetate in hexanes) afforded the title compound as a pale yellow solid. (2.1 g, 51%). ¹H NMR (300 MHz, DMSO-d6) δ 10.3 (1H, br s), 7.95 (1H, s), 7.7-7.6 (1H, app t), 7.59 (1H, s), 7.1-7.2 (1H, m), 5.12 (s, 2H), 1.23 (s, 12H).

Part B: A mixture of Example 1, Part D (1.49 g), boronate from Example 7, Part A. (2.13 g), PdCl2(dppf) (0.398 g), potassium phosphate (2.07 g), in DME (45 mL) and water (5 mL) was heated at 95 C overnight. The reaction was allowed to cool, diluted with ethyl acetate and filtered through Celite. The filtrate was washed with water, brine and dried (sodium sulfate). Chromatography afforded the title compound.

Part C: A solution of the compound from Example 7, Part B (260 mg) in THF (25 mL) at rt was added MCPBA (288 mg) in one portion. After 1 hr at rt, ethyl acetate was added and was washed with sat. sodium bicarbonate (2×), brine and dried (sodium sulfate). Concentration afforded the title compound that was used without further purification.

Part D: A solution of the compound from Example 7, Part C (1 equiv), amine (5 equiv), DIEA (5 equiv) in NMP was heated at 50 C overnight. The reaction mixture was concentrated and purified by Prep-LC. Using this general procedure compounds listed in Table 2 (shown immediately below) were prepared.

TABLE 2

| | | | MS | |
|---|---|---|---|---|
| | | | MH⁺ | HPLC |
| Example | Column 2 | MW | m/z | t_{R} |
| 7-1 | 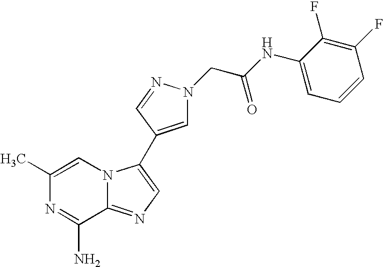 | 383.35 | 384.1 | 3.01 |
| 7-2 | 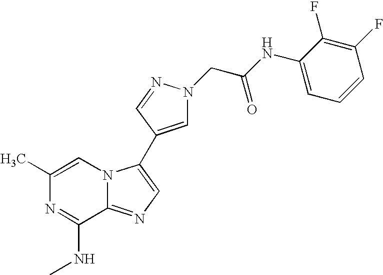 | 397.38 | 398.2 | 2.59 |
| 7-3 | 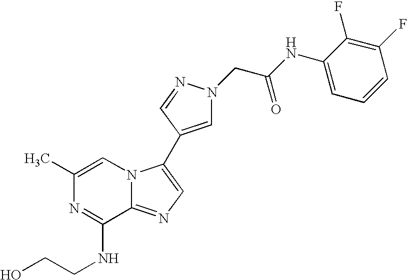 | 427.4 | 428.2 | 2.57 |
| 7-4 | 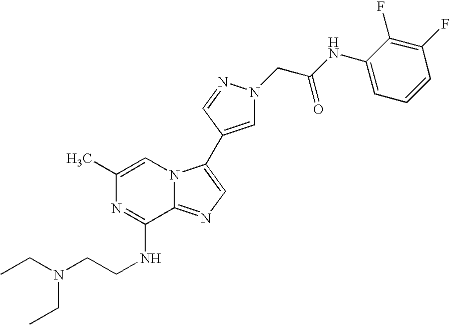 | 482.53 | 483.3 | 2.90 |
| 7-5 | 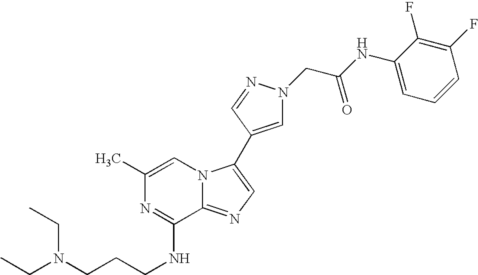 | 496.56 | 497.3 | 2.83 |
| 7-6 | 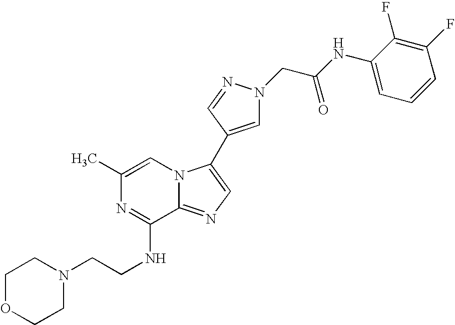 | 496.51 | 497.2 | 3.02 |
| 7-7 | 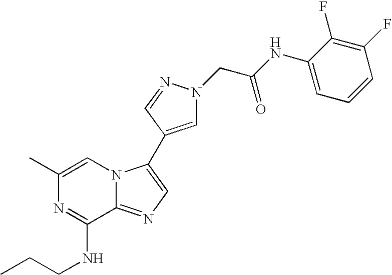 | 425.43 | 426.1 | 3.00 |
| 7-8 | 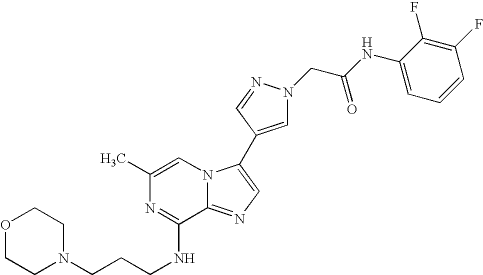 | 510.54 | 511.1 | 2.38 |
| 7-9 | 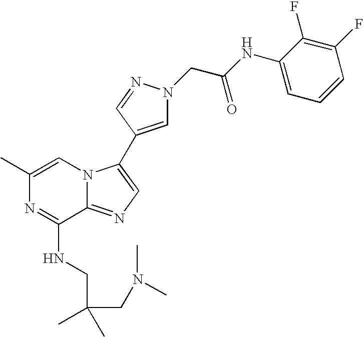 | 496.56 | 497.2 | 2.71 |
| 7-10 | 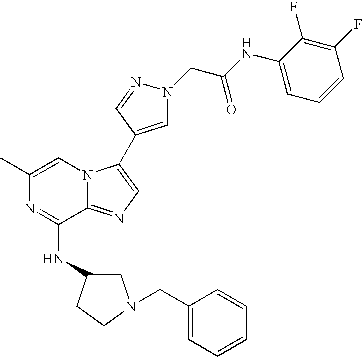 | 542.58 | 543.2 | 3.22 |
| 7-11 | 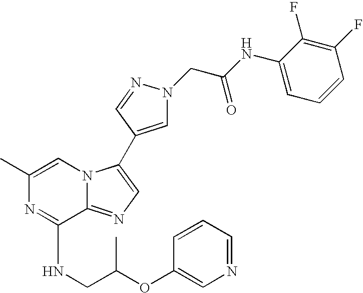 | 518.52 | 519.2 | 2.39 |
| 7-12 | 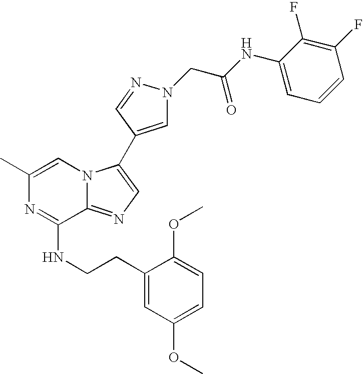 | 547.56 | 548.2 | 4.29 |
| 7-13 | 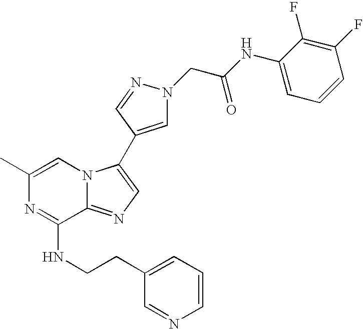 | 488.49 | 489.1 | 2.44 |
| 7-14 | 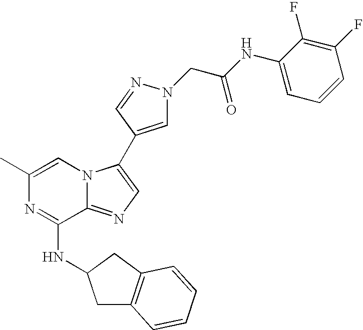 | 499.51 | 500.2 | 3.36 |
| 7-15 | 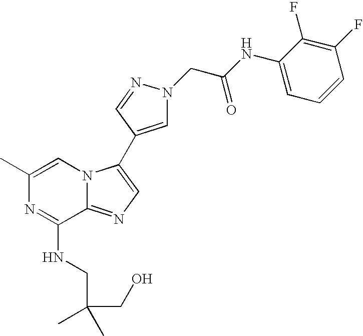 | 469.49 | 470.2 | 3.09 |
| 7-16 | 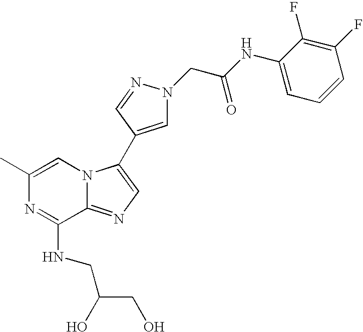 | 457.43 | 458.2 | 2.95 |
| 7-17 | 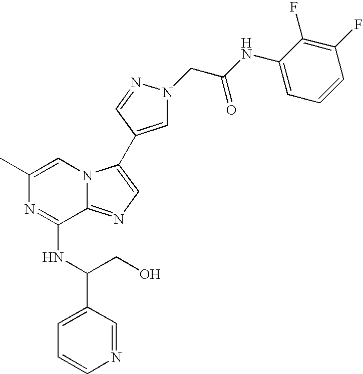 | 504.49 | 505.2 | 2.31 |
| 7-18 | 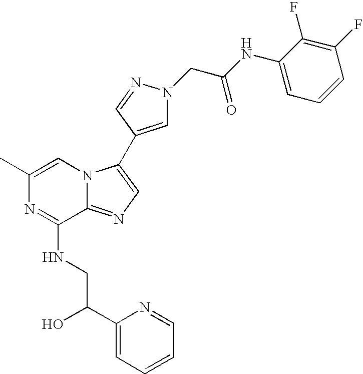 | 504.49 | 505.1 | 2.60 |
| 7-19 | 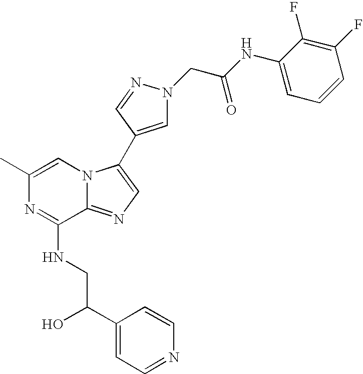 | 504.49 | 505.1 | 2.37 |
| 7-20 | 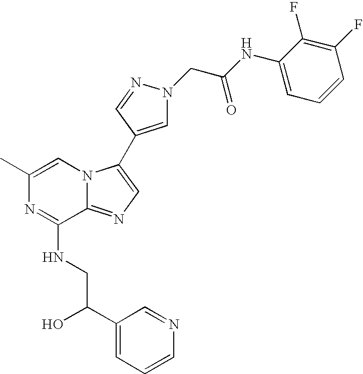 | 504.49 | 505.1 | 2.15 |
| 7-21 | 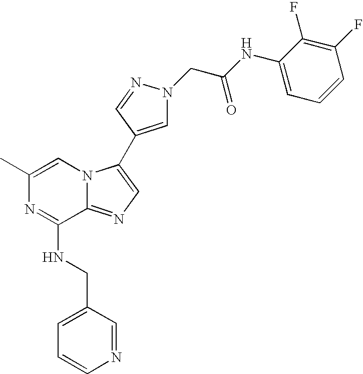 | 474.47 | 475.1 | 2.56 |
| 7-22 | 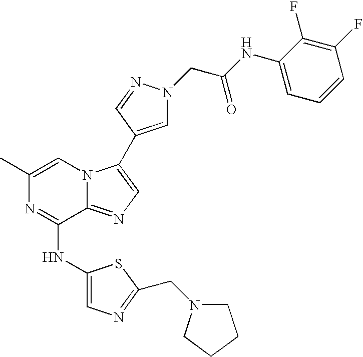 | 549.2 | 550.2 | 3.12 |
| 7-23 | 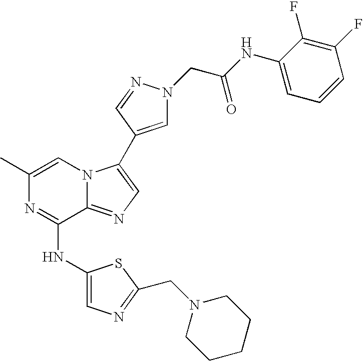 | 563.2 | 564.2 | 3.21 |
| 7-24 | 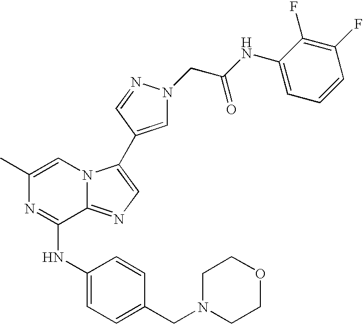 | 558.2 | 559.2 | 1.42 |
| 7-25 | 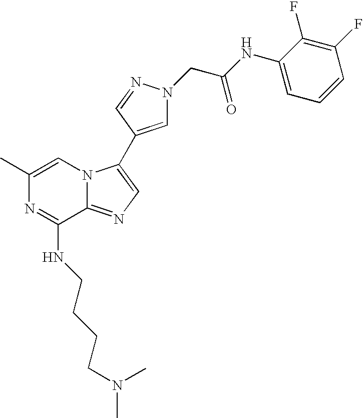 | 482.53 | 483.3 | 2.36 |

### Example 8

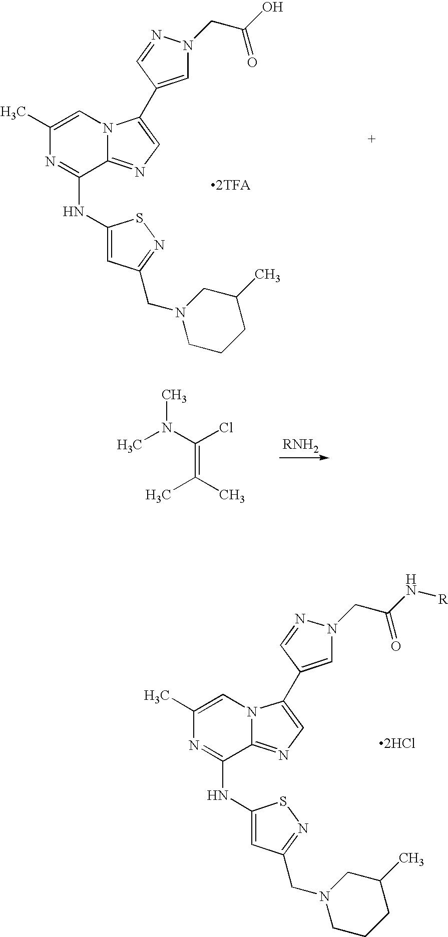

To a stirring suspension of carboxylic acid (1 equiv) in dichloromethane at 0° C. was added 1-chloro-N,N-2-trimethyl-1-propenylamine (5 equiv). After stirring for 1 hour the amine was added as a solution in dichloromethane or pyridine. When the reaction was deemed complete by HPLC analysis the mixture was concentrated under reduced pressure. Purification by prep-HPLC and conversion to the hydrochloride salt provided the compounds listed in Table 3 (shown immediately below).

TABLE 3

| | | | MS | |
|---|---|---|---|---|
| | | | MH⁺ | HPLC |
| Example | Column 2 | MW | m/z | t_{R} |
| 8-1 | 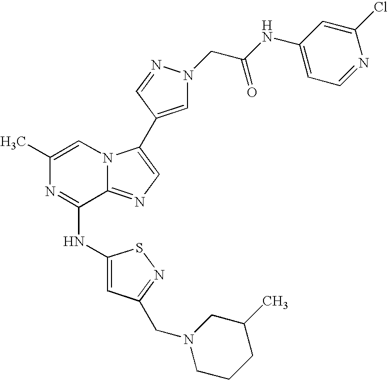 | 577.1 | 587.0 | 4.64 |
| 8-2 | 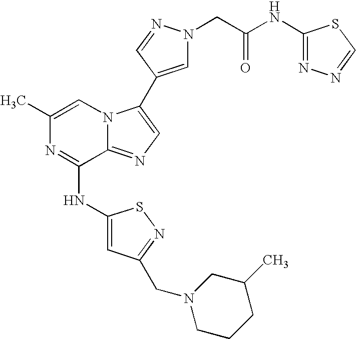 | 549.6 | 550.9 | 4.11 |
| 8-3 | 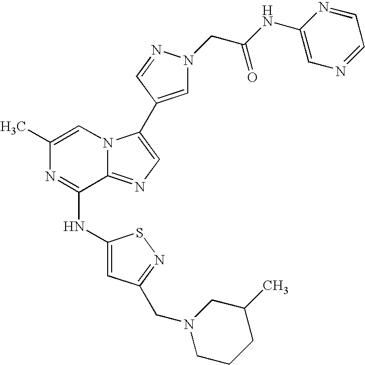 | 543.6 | 544.6 | 4.15 |
| 8-4 | 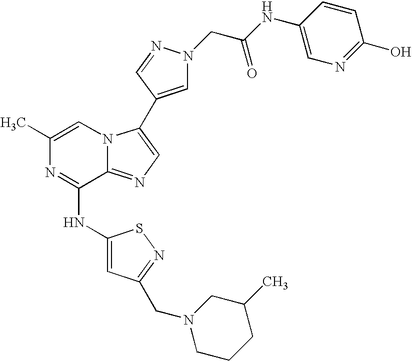 | 558.6 | 559.4 | 3.73 |
| 8-5 | 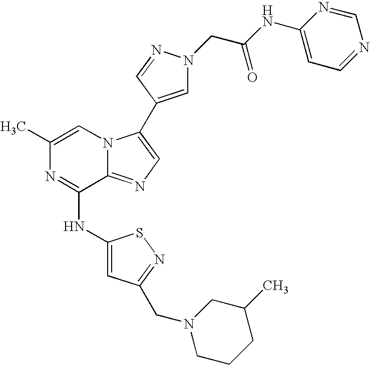 | 543.6 | 544.5 | 6.99 |
| 8-6 | 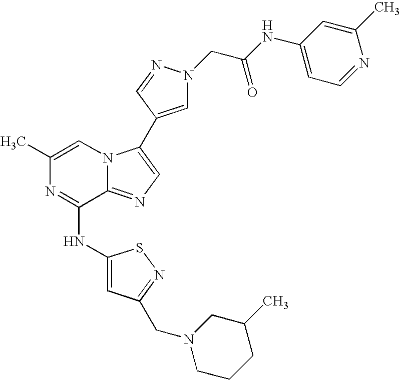 | 556.6 | 557.3 | 3.73 |

### Example 9

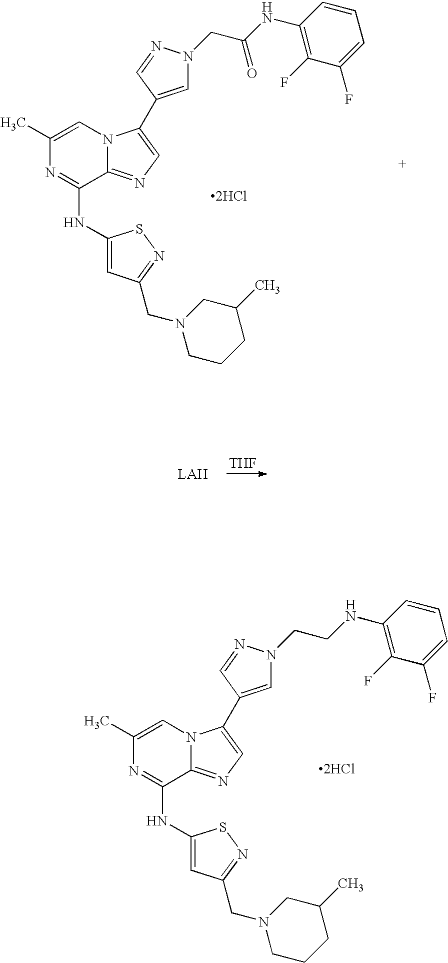

Lithium aluminum hydride (2 mg, 2.2 equiv) was added to a stirring suspension of amide (14 mg, 1 equiv) in THF (1 mL) at room temperature. HPLC analysis after 15 minutes showed no starting material so reaction was quenched with methanol, concentrated under reduced pressure, purified by prep-HPLC, and conversion to the hydrochloride salt afforded the title compound as a white solid 6.5 mg (47%)). ¹H NMR (300 MHz, DMSO-d₆) δ 12.3 (s, 1H), 10.1 (bs, 1H), 8.41 (s, 1H), 8.03 (s, 1H), 7.89 (m, 2H), 7.30 (s, 1H), 6.97 (m, 1H), 6.55 (m, 2H), 4.39 (m, 4H), 3.67 (m, 2H), 3.39 (m, 2H), 2.80 (m, 2H), 2.48 (s, 3H), 1.79 (m, 4H), 1.05 (m, 1H), 0.89 (m, 3H). HPLC t_{R}=5.58 min (UV₂₅₄ₙₘ). Mass calculated for formula C₂₈H₃₁F₂N₉S 563.24; observed MH⁺ (ESI MS) 564.8 (m/z).

### Example 10

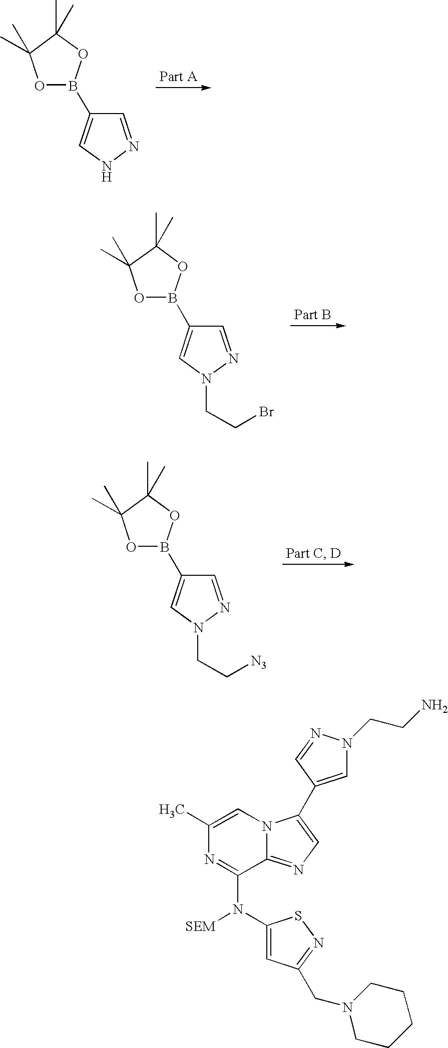

Part A: Potassium hydroxide (1.45 g, 10.0 equiv) was added to a stirring solution of 4-(4,4,5,5-Tetramethyl-1,3,2-dioxaborolan-2-yl)-1H-pyrazole (500 mg, 1.00 equiv) in DMSO (5 mL) at room temperature. After stirring for 1 hour, 1,2-dibromoethane (9.69 g, 20.0 equiv) was added. The reaction was stirred for 16 hours at which time TLC indicated no starting material remained so the reaction was quenched with water (10 mL) and extracted with ethyl acetate (3×15 mL). The combined organics were washed with brine (20 mL), dried over sodium sulfate, filtered, concentrated under reduced pressure, and purification by silica gel chromatography (12 g SiO₂, dichloromethane to 5% methanol in dichloromethane) afforded the desired boronate as a yellow oil 350 mg (45%).

Part B: A mixture of the boronate from example 10 part A (250 mg, 1.00 equiv), sodium azide (108 mg, 2.00 equiv), and sodium iodide (124 mg, 1.00 equiv) in DMSO (2 mL) was stirred at 50° C. for 2 hours at which time TLC indicated no starting material remained. The reaction was allowed to cool to room temperature, then quenched with water (8 mL) and extracted with ethyl acetate (3×15 mL). The combined organics were washed with brine (15 mL), dried over sodium sulfate, filtered, concentrated under reduced pressure, and purification by silica gel chromatography (12 g SiO₂, dichloromethane to 5% methanol in dichloromethane) afforded the desired boronate as a yellow solid 170 mg (78%).

Part C: To a mixture of aryl iodide from Part B (315 mg, 1.00 equiv), PdCl₂(dppf) (39 mg, 0.10 equiv), and potassium phosphate (228 mg, 2.00 equiv) under nitrogen was added a solution of the boronate from Part B (170 mg, 1.20 equiv) in 1,4-dioxane (1.5 mL), followed by water (0.15 mL). The mixture was stirred at 90° C. for 17 hours at which time TLC indicated no starting material remained. The reaction was allowed to cool to room then diluted with ethyl acetate (8 mL) and washed with brine (10 mL), dried over sodium sulfate, filtered, concentrated under reduced pressure, and purification by silica gel chromatography (12 g SiO₂, dichloromethane to 5% methanol in dichloromethane) afforded the desired azide as a brown solid 125 mg (39%).

Part D: To a stirring solution of azide from Part C (125 mg, 1.00 equiv) in 1,4-dioxane (2 mL) and water (0.2 mL) was added poly-styrene bound triphenylphosphine (84 mg, 1.20 equiv). The reaction was stirred at room temperature for 3 days at which time TLC indicated no starting material remained. The mixture was filtered, the mother liquor concentrated under reduced pressure, and the resulting residue purified by silica gel chromatography (12 g SiO₂, dichloromethane to 10% methanol in dichloromethane) affording the desired amine as a brown oil 77 mg (64%).

### Example 11

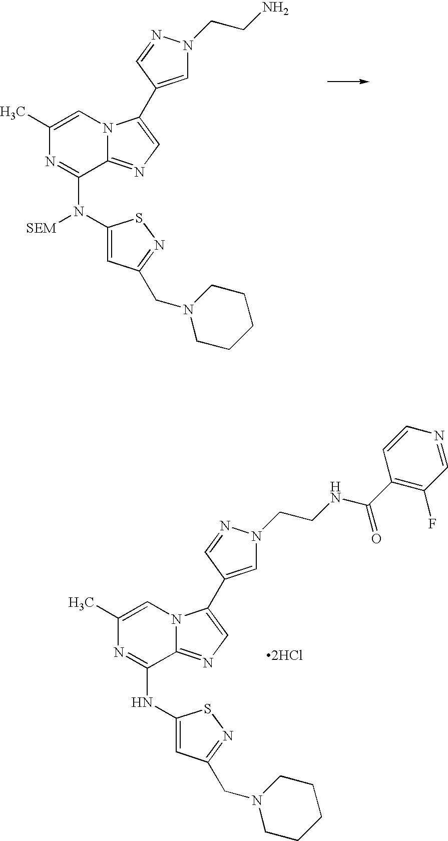

N-Methyl morpholine (14 mg, 2.00 equiv) was added to a stirring mixture of carboxylic acid (14 mg, 1.50 equiv) and HATU (39 mg, 1.50 equiv) in DMF (0.5 mL). After 30 minutes, the amine from example 10 (38.5 mg, 1.00 equiv) was added as a solution in DMF (0.5 mL). The mixture was stirred for 5 hours at which time HPLC indicated no starting material remained. The mixture was concentrated and the residue dissolved in 1,4-dioxane (1 mL). A solution on HCl in dioxane (1 mL, 4M in dioxane) was added and the mixture was sonicated for 1.5 hours at which time HPLC indicated no starting material remained. The mixture was concentrated under reduced pressure, purified by prep-HPLC, and conversion to the HCl salt afforded the desired compound as a yellow solid 6 mg (14%). ¹H NMR (300 MHz, CD₃OD) δ 8.90 (m, 1H), 8.72 (m, 1H), 8.36 (s, 1H), 8.24 (s, 1H), 8.01 (m, 3H), 7.32 (s, 1H), 4.58 (m, 2H), 4.42 (s, 2H), 3.94 (m, 2H), 3.60 (m, 2H), 3.09 (m, 2H), 2.61 (s, 3H), 1.81 (m, 6H), 1.55 (m, 2H). HPLC t_{R}=3.97 min (UV₂₅₄ₙₘ). Mass calculated for formula C₂₇H₂₉FN₁₀OS 560.22; observed MH⁺ (ESI MS) 561.3 (m/z).

### Example 12

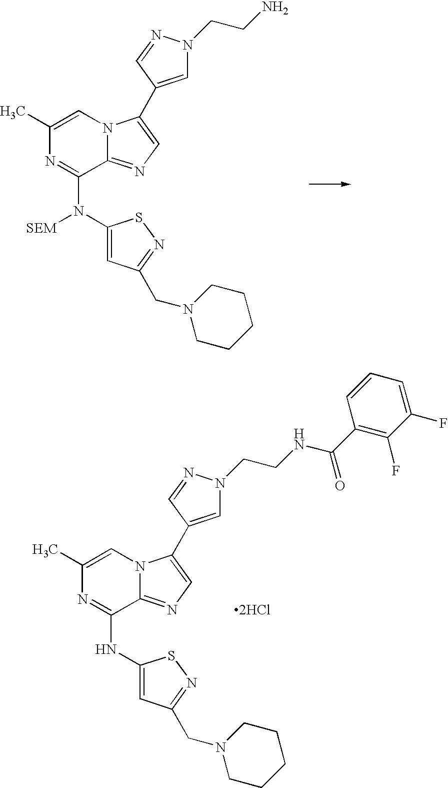

To a stirring solution of amine from example 10 (38.5 mg, 1.00 equiv) and triethylamine (14 mg, 2.00 equiv) in dichloromethane (0.75 mL) was added 2,3-difluorobenzoylchloride (13 mg, 1.10 equiv) dropwise. HPLC analysis after 4 hours showed no starting material so reaction was quenched with saturated aqueous sodium bicarbonate (2 mL) and then extracted with dichloromethane (3×1 mL). The combined organics were concentrated and the resulting residue was dissolved in 1,4-dioxane (1 mL) and a solution on HCl in dioxane (1 mL, 4M in dioxane) was added and the mixture was sonicated for 1.5 hours at which time HPLC indicated no starting material remained. The mixture was concentrated under reduced pressure, purified by prep-HPLC, and conversion to the HCl salt afforded the desired compound as a yellow solid 5 mg (11%). ¹H NMR (300 MHz, CD₃OD) δ 8.34 (s, 1H), 8.21 (s, 1H), 8.06 (s, 1H), 7.92 (s, 1H), 7.44 (m, 2H), 7.36 (2, 1H), 7.20 (m, 1H), 4.56 (m, 2H), 4.41 (s, 2H), 3.92 (m, 2H), 3.59 (m, 2H), 3.08 (m, 2H), 2.59 (s, 3H), 1.78 (m, 6H). HPLC t_{R}=4.61 min (UV₂₅₄ₙₘ). as calculated for formula C₂₈H₂₉F₂N₉OS 577.22; observed MH⁺ (ESI MS) 578.8 (m/z).

### Example 13

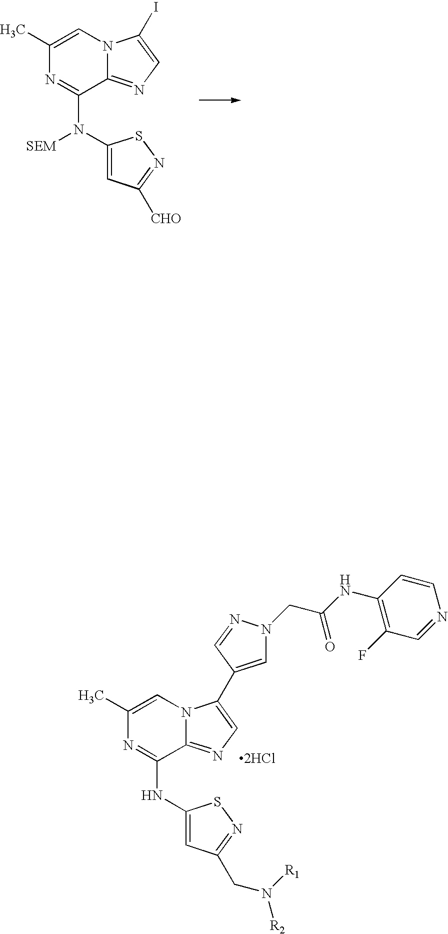

Sodium triacetoxyborohydride (1.50 equiv) was added to a stirring mixture of aldehyde (1.00 equiv), amine (1.20 equiv), and acetic acid (1.00 equiv) in 1,2-dichloroethane at room temperature. The mixture was stirred until no starting material remained as judged by TLC. The reaction was then quenched with 1N NaOH and extracted three times with chloroform. The combined organics were dried over sodium sulfate, filtered, and concentrated. The residue was then added as a solution in dioxane (1 equiv) to a mixture of boronate (1.50 equiv), PdCl₂(dppf) (0.10 equiv), and potassium phosphate (2.00 equiv) under nitrogen. Water was added and the mixture was stirred at 90° C. for 17 hours at which time HPLC indicated no starting material remained. The reaction was allowed to cool to room temperature the diluted with ethyl acetate, washed with water, dried over sodium sulfate, filtered, concentrated under reduced pressure, and purification by silica gel chromatography afforded the coupled product. This material was dissolved in 1,4-dioxane, HCl (4N in dioxane) was added and the mixture was sonicated until such time that HPLC indicated no starting material remained. The mixture was concentrated under reduced pressure, purified by prep-HPLC, and conversion to the hydrochloride salt afforded the title compounds as white solids in Table 4 (shown immediately below):

TABLE 4

| | | | MS | |
|---|---|---|---|---|
| | | | MH⁺ | HPLC |
| Example | Column 2 | MW | m/z | t_{R} |
| 13-1 | 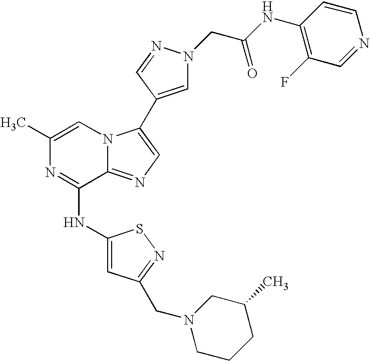 | 560.6 | 561.4 | 3.75 |
| 13-2 | 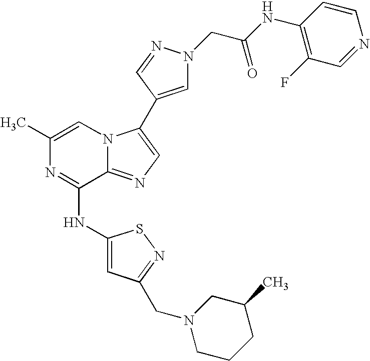 | 560.6 | 561.3 | 3.78 |
| 13-3 | 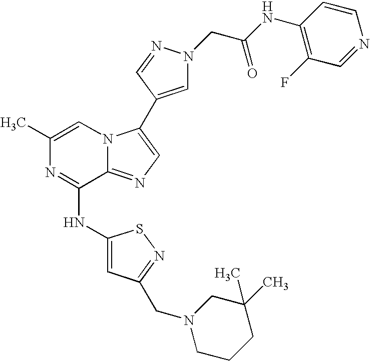 | 574.6 | 575.6 | 3.97 |
| 13-4 | 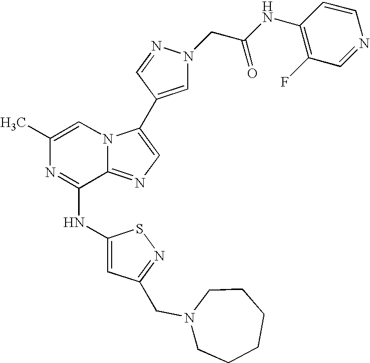 | 560.6 | 561.4 | 3.76 |
| 13-5 | 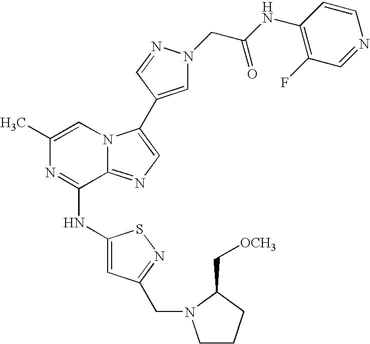 | 576.6 | 578.2 | 3.63 |
| 13-6 | 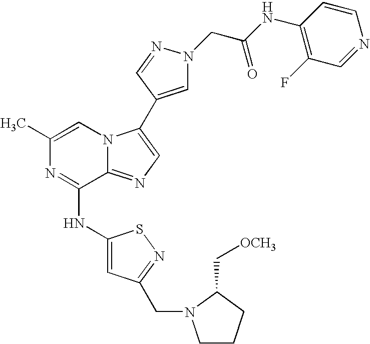 | 576.6 | 578.2 | 3.62 |
| 13-7 | 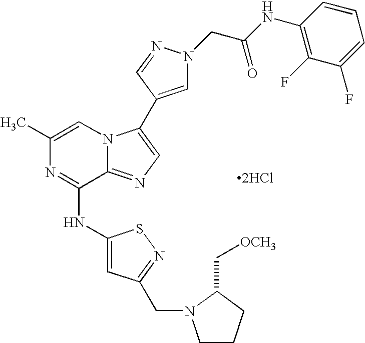 | 593.7 | 594.7 | 5.13 |
| 13-8 | 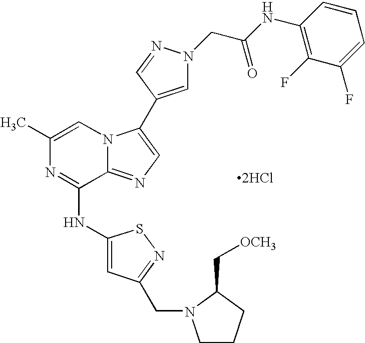 | 593.7 | 594.7 | 5.15 |

### Example 14

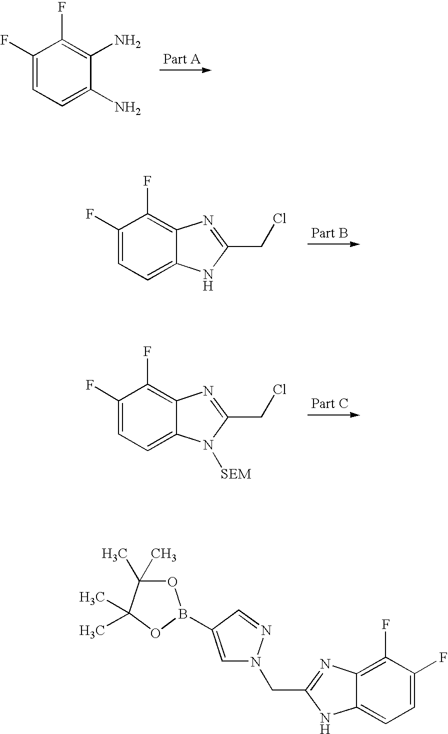

Part A: Chloroacetic acid (5.11 g, 1.3 equiv) was added to a stirring solution of 2-amino-3,4-difluoroaniline (6.00 g, 1 equiv) in 6 N hydrochloric acid (28 mL). After stirring for 18 hours at 95° C. the reaction was cooled to room temperature, made basic with 10% aqueous potassium carbonate and extracted with ethyl acetate (850 mL). The organic layer was separated, dried over sodium sulfate, filtered, concentrated under reduced pressure, and purification by silica gel chromatography (120 g SiO₂, hexanes to 60% ethyl acetate in hexanes) afforded the desired benzimidazole as a pink solid 6.24 g (74%).

Part B: A mixture of the benzimidazole from example 14 part A (4.18 g, 1.00 equiv), potassium carbonate (8.53 g, 3.00 equiv) in DMF (50 mL) was stirred at room temperature for 5 minutes at which time 2-(trimethylsilyl)ethoxymethyl chloride (4.0 mL, 1.1 equiv) was added. After stirring at room temperature for 18 hours the reaction was quenched with a saturated aqueous solution of sodium bicarbonate (40 mL) and concentrated under reduced pressure to a residue. The residue was diluted with ethyl acetate (500 mL) and washed with water (150 mL), dried over sodium sulfate, filtered, concentrated under reduced pressure, and purification by silica gel chromatography (120 g SiO₂, hexanes to 50% of ethyl acetate in hexanes) afforded the desired benzimidazole as a brown oil 3.11 g (45%).

Part C: To a solution of 4-(4,4,5,5-Tetramethyl-1,3,2-dioxaborolan-2-yl)-1H-pyrazole (1.65 g, 1 equiv), benzimidazole from Example 14 part B (3.11 g, 1.1 equiv) in DMA (57 mL) was added potassium carbonate (3.51 g, 3 equiv). The mixture was heated at 50° C. for 18 hours. The reaction mixture was poured into water (250 mL), extracted with ethyl acetate (500 mL), the organic layer washed with brine (250 mL), dried over sodium sulfate, filtered, concentrated under reduced pressure, and purification by silica gel chromatography (80 g SiO2, hexanes to 50% of ethyl acetate in hexanes) afforded the desired boronate as a off-white solid 2.67 g (64%).

### Example 15

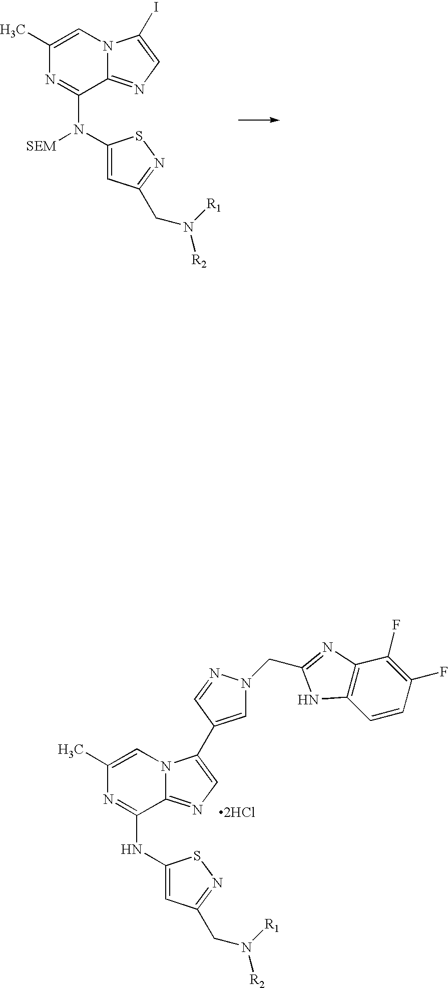

To a mixture of aryl iodide (100 mg, 1.00 equiv), PdCl₂(dppf) (12 mg, 0.10 equiv), and potassium phosphate (71 mg, 2.00 equiv) under nitrogen was added a solution of the boronate from Example 14 part C (123 mg, 1.20 equiv) in 1,4-dioxane (2.0 mL), followed by water (0.2 mL). The mixture was stirred at 90° C. for 18 hours at which time TLC indicated no starting material remained. The reaction was allowed to cool to room temperature, then diluted with ethyl acetate (100 mL) and washed with water (30 mL), dried over sodium sulfate, filtered, concentrated under reduced pressure, and purification by silica gel chromatography (12 g SiO₂, dichloromethane to 10% methanol in dichloromethane) afforded the desired coupled product.

This material was dissolved in 1,4-dioxane, HCl (4N in dioxane) was added and the mixture was sonicated until such time that HPLC indicated no starting material remained. The mixture was concentrated under reduced pressure, purified by prep-HPLC, and conversion to the hydrochloride salt afforded the title compounds as off-white solids in Table 5 (shown immediately below):

TABLE 5

| | | | MS | |
|---|---|---|---|---|
| | | | MH⁺ | HPLC |
| Example | Column 2 | MW | m/z | t_{R} |
| 15-1 | 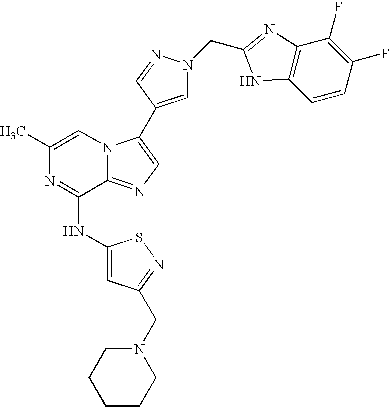 | 560.2 | 561.2 | 4.74 |
| 15-2 | 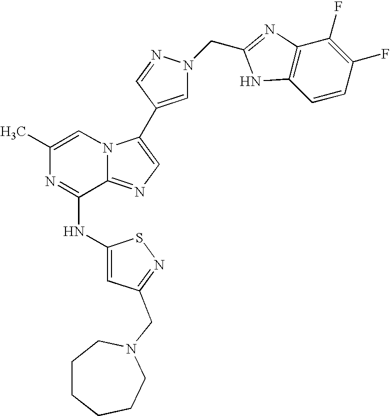 | 574.2 | 575.7 | 4.99 |

### Example 16

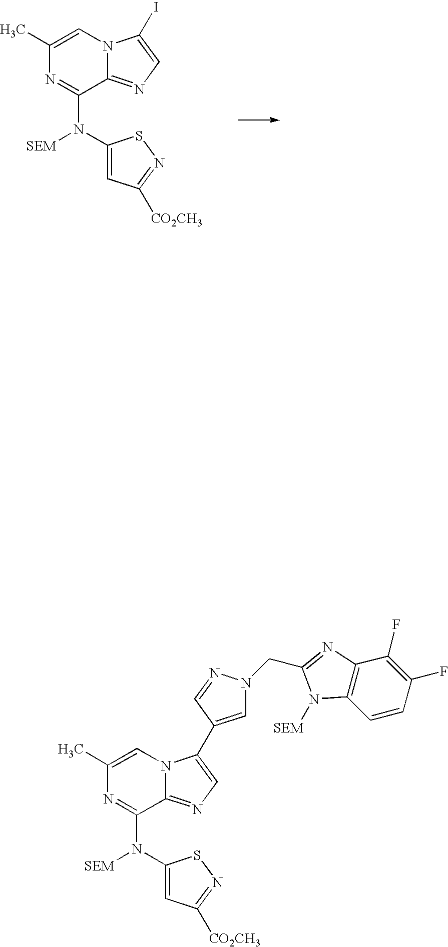

To a mixture of aryl iodide (2.15 g, 1.00 equiv), PdCl₂(dppf) (288 mg, 0.10 equiv), and potassium phosphate (1.67 mg, 2.00 equiv) under nitrogen was added a solution of the boronate from Example 14 part C (2.51 g, 1.20 equiv) in 1,4-dioxane (47 mL), followed by water (4.7 mL). The mixture was stirred at 90° C. for 3 hours at which time TLC indicated no starting material remained. The reaction was allowed to cool to room then diluted with ethyl acetate (700 mL) and washed with water (250 mL), dried over sodium sulfate, filtered, concentrated under reduced pressure, and purification by silica gel chromatography (120 g SiO₂, hexanes to 100% ethyl acetate) afforded the desired coupled product as a brown foam 2.04 g (66%).

### Example 17

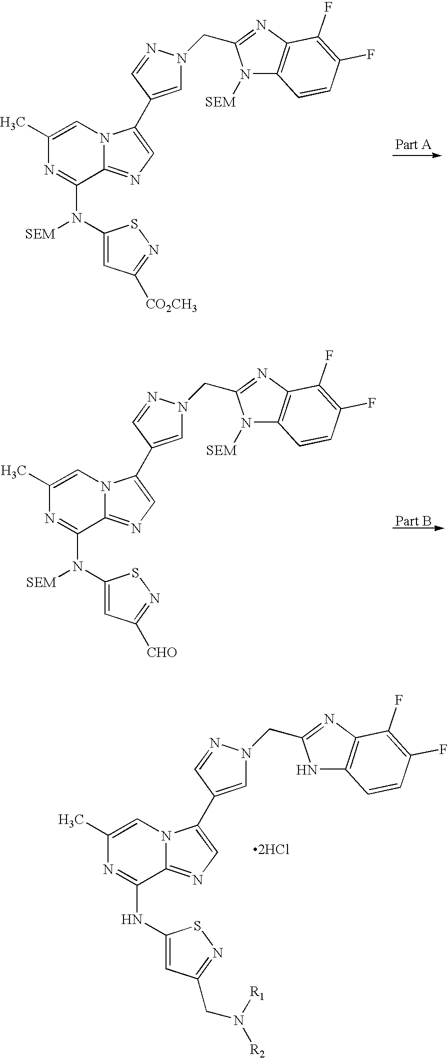

Part A: To a stirring solution of the coupled product from Example 16 (2.04 g, 1 equiv) in tetrahydrofuran (63 mL) at −78° C. was added DIBAL-H (1M in dichloromethane, 6.5 mL, 2.5 equiv) dropwise. The mixture was stirred at −78° C. for 5 hours at which time thin layer chromatography (30% ethyl acetate/hexanes) indicated the reaction was complete. The mixture was quickly poured into stirring saturated aqueous sodium potassium tartrate and stirred at room temperature for 14 hours. The mixture was extracted with ethyl acetate (500 mL), the organic layer separated, dried over sodium sulfate, filtered, and concentrated under reduced pressure affording the aldehyde as a brown foam 1.96 g (100%).

Part B: Sodium triacetoxyborohydride (1.50 equiv) was added to a stirring mixture of aldehyde (1.00 equiv), amine (1.20 equiv), and acetic acid (1.00 equiv) in 1,2-dichloroethane at room temperature. The mixture was stirred until no starting material remained as judged by TLC. The reaction was then quenched with 1N NaOH and extracted three times with chloroform. The combined organics were dried over sodium sulfate, filtered, and concentrated. This material was dissolved in 1,4-dioxane, HCl (4N in dioxane) was added and the mixture was sonicated until such time that HPLC indicated no starting material remained. The mixture was concentrated under reduced pressure, purified by prep-HPLC, and conversion to the hydrochloride salt afforded the title compounds as off-white solids in Table 6 (shown immediately below):

TABLE 6

| | | | MS | |
|---|---|---|---|---|
| | | | MH⁺ | HPLC |
| Example | Column 2 | MW | m/z | t_{R} |
| 17-1 | 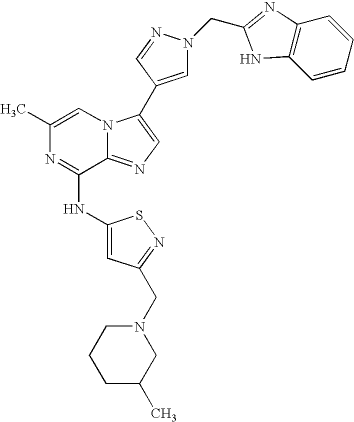 | 538.6 | 539.8 | 4.08 |
| 17-2 | 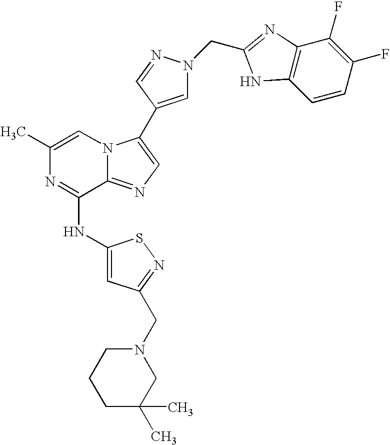 | 588.23 | 589.1 | 4.84 |
| 17-3 | 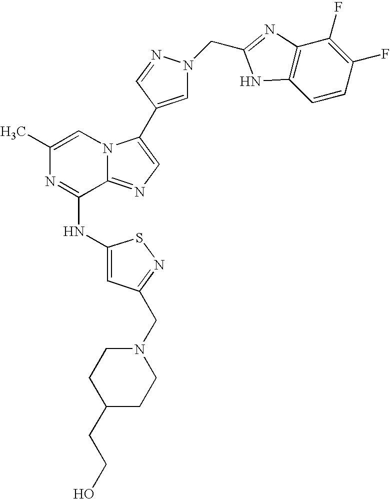 | 604.2 | 605.9 | 4.35 |
| 17-4 | 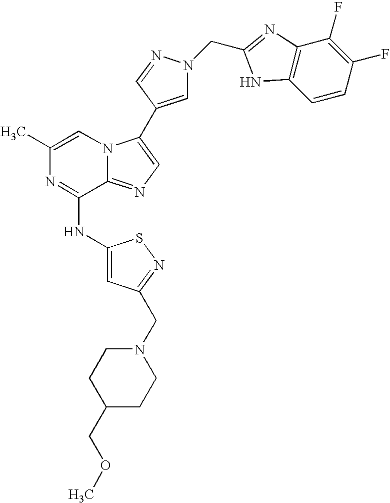 | 604.2 | 605.2 | 4.38 |

### Example 18

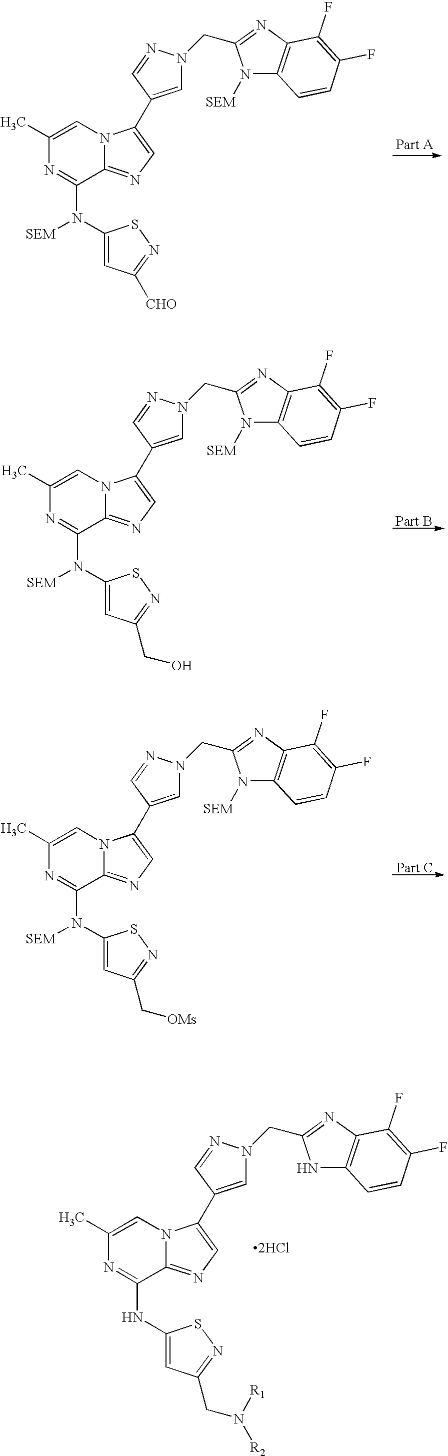

Part A: Sodium borohydride (2.0 equiv) was added to a stirring mixture of aldehyde from Example 17 part A (1.00 equiv) in acetic acid (5.7 mL) in 1,2-dichloromethane (17 mL) at room temperature. The mixture was stirred until no starting material remained as judged by TLC. The reaction was then quenched with 2N NaOH (11 mL) and a saturated solution of aqueous sodium bicarbonate (35 mL). After stirring at room temperature for 15 minutes, the phases were separated and the organic layer was dried over sodium sulfate, filtered and concentrated under reduced pressure to afford the alcohol as a brown foam 1.01 g (100%).

Part B: Methane sulfonyl chloride (2 equiv) was added to a stirring solution of alcohol from Example 18 part A (1 equiv) and triethylamine (4 equiv) in THF (40 mL). After stirring at room temperature for 30 minutes the reaction was quenched with a saturated aqueous solution of ammonium chloride (14 mL) and water (14 mL), extracted with dichloromethane (2×80 mL), dried over sodium sulfate, filtered and concentrated under reduced pressure affording the mesylate as a brown foam 1.04 g (90%).

Part C: To a stirring solution of mesylate from Example 18 part B (1 equiv), amine (3 equiv), sodium iodide (0.5 equiv) in THF (1.0 mL) was added diisopropylethylamine (3 equiv) and the reaction heated at 60° C. for 18 hours. The reaction was cooled to room temperature, diluted with dichloromethane (50 mL) and the organic layer washed with water (30 mL), brine (30 mL), dried over sodium sulfate, filtered and concentrated under reduced pressure. This material was dissolved in 1,4-dioxane, HCl (4N in dioxane) was added and the mixture was sonicated until such time that HPLC indicated no starting material remained. The mixture was concentrated under reduced pressure, purified by prep-HPLC, and conversion to the hydrochloride salt afforded the title compounds as off-white solids in Table 7 (shown immediately below):

TABLE 7

| | | | MS | |
|---|---|---|---|---|
| | | | MH⁺ | HPLC |
| Example | Column 2 | MW | m/z | t_{R} |
| 18-1 | 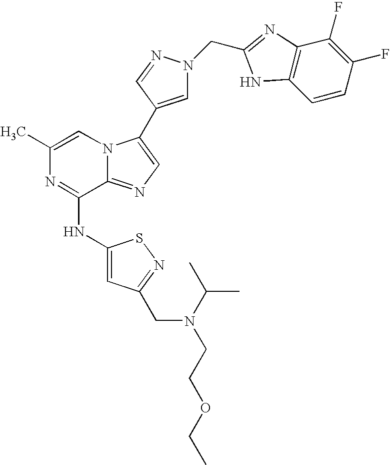 | 606.2 | 607.1 | 4.95 |
| 18-2 | 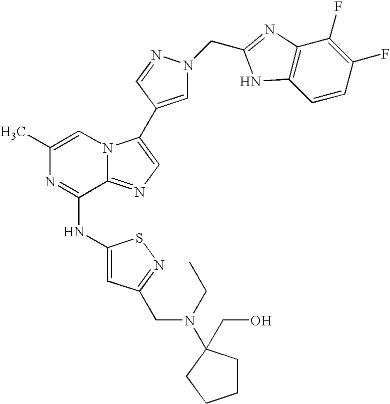 | 618.2 | 619.9 | 4.67 |
| 18-3 | 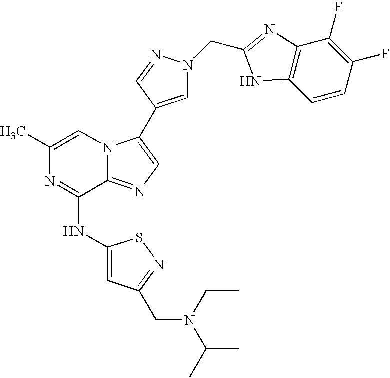 | 562.2 | 563.5 | 4.48 |
| 18-4 | 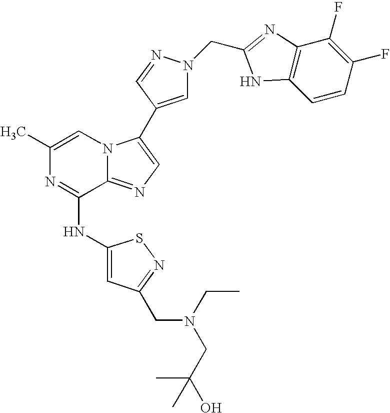 | 592.2 | 593.5 | 4.37 |

### Example 19

The following compounds in Table 8 (shown immediately below) were prepared by a method similar to the method described in Example 18.

TABLE 8

| | | | MS | |
|---|---|---|---|---|
| | | | MH⁺ | HPLC |
| Example | Column 2 | MW | m/z | t_{R} |
| 19-1 | 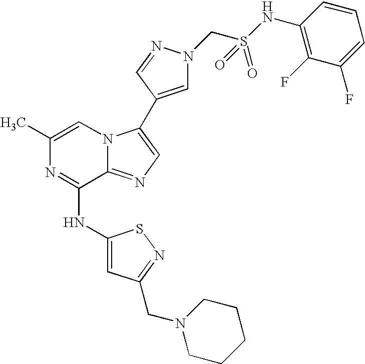 | 599.1 | | |
| 19-2 | 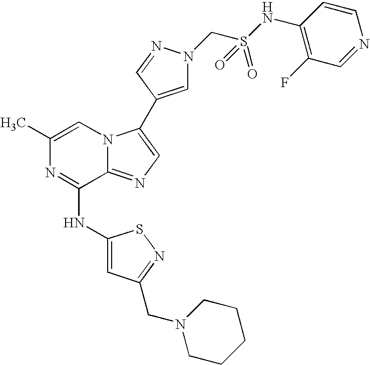 | 582.1 | | |
| 19-3 | 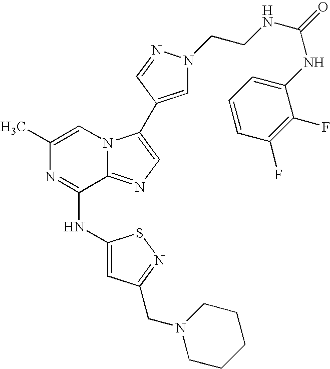 | 592.2 | | |
| 19-4 | 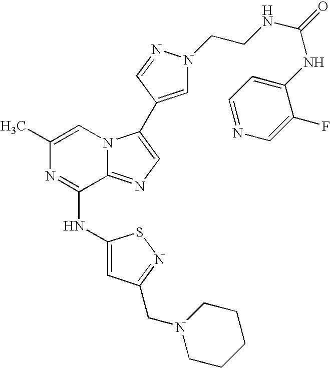 | 575.2 | | |
| 19-5 | 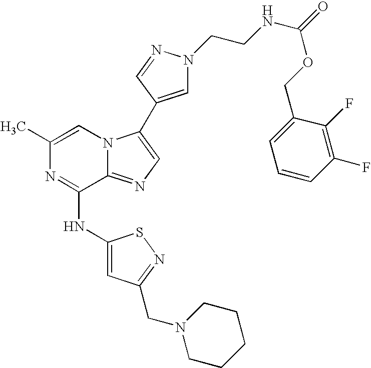 | 607.2 | | |
| 19-6 | 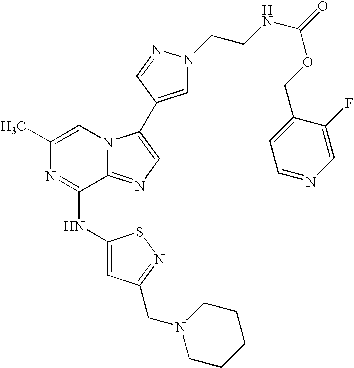 | 590.6 | | |
| 19-7 | 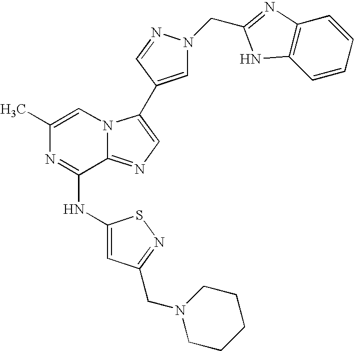 | 525.2 | | |
| 19-8 | 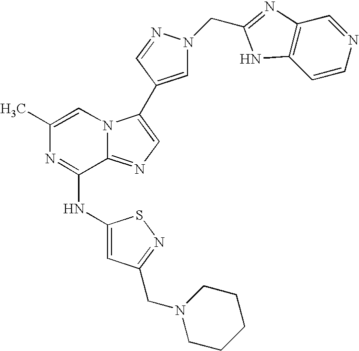 | 525.2 | | |
| 19-9 | 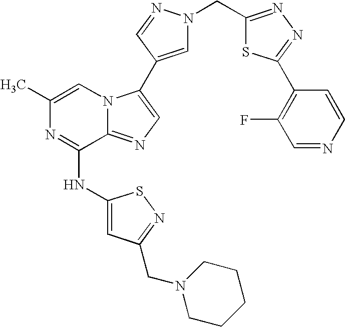 | 587.1 | | |
| 19-10 | 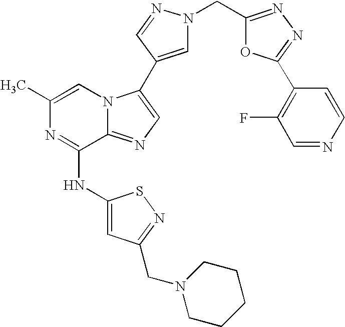 | 571.2 | | |
| 19-11 | 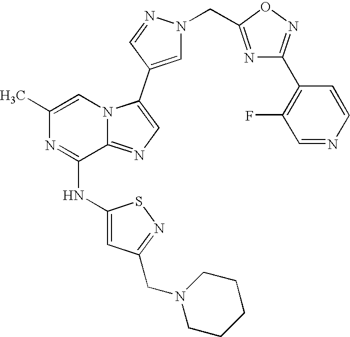 | 571.2 | | |
| 19-12 | 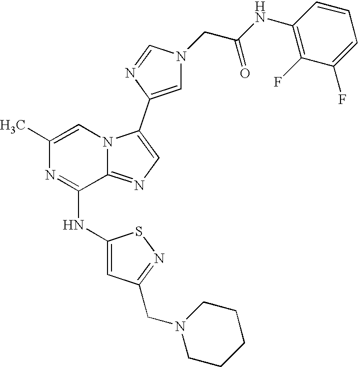 | 563.2 | | |
| 19-13 | 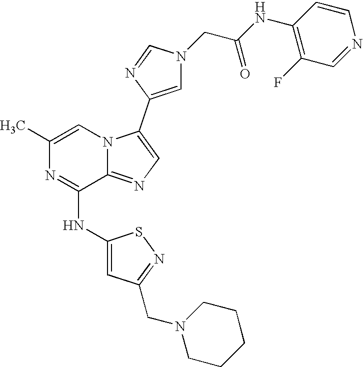 | 546.2 | | |
| 19-14 | 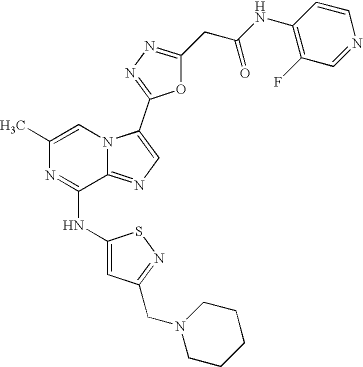 | 548.1 | | |
| 19-15 | 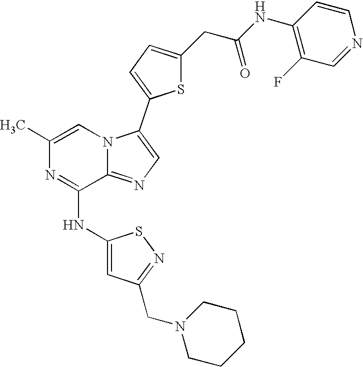 | | | |
| 19-17 | 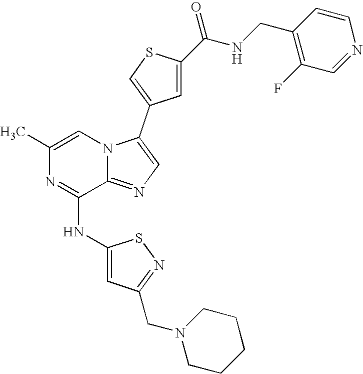 | | | |
| 19-16 | 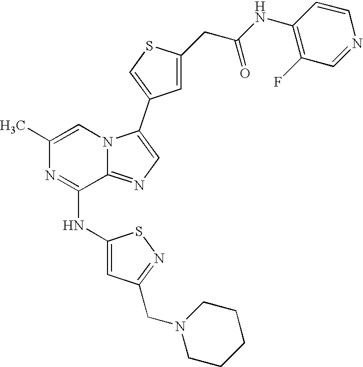 | | | |
| 19-17 | 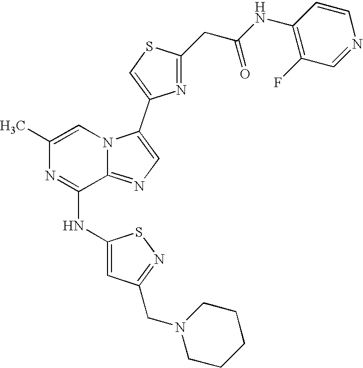 | | | |
| 19-18 | 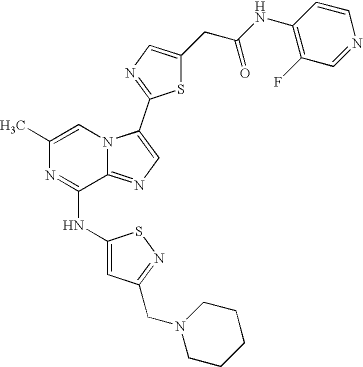 | | | |
| 19-19 | 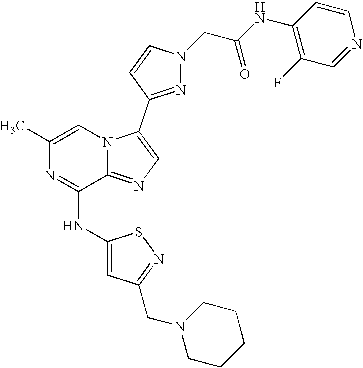 | | | |
| 19-20 | 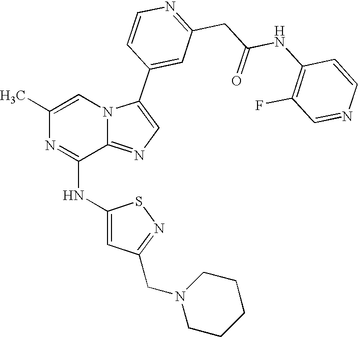 | | | |
| 19-21 | 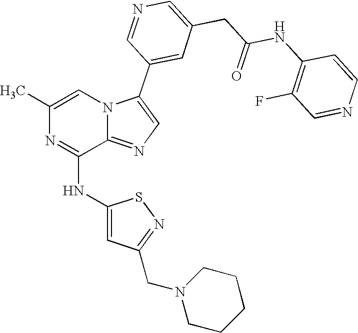 | | | |
| 19-22 | 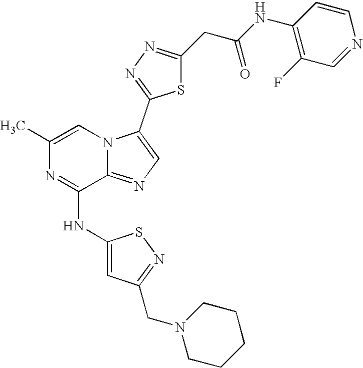 | 564.1 | | |
| 19-23 | 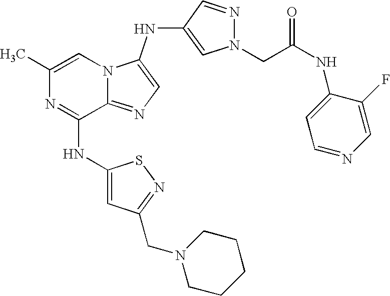 | 561.6 | | |
| 19-24 | 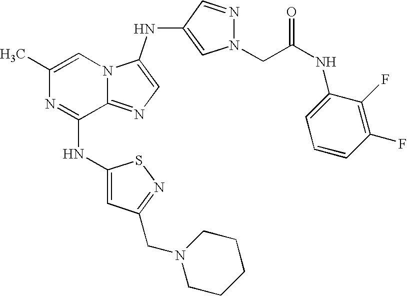 | 578.6 | | |
| 19-25 | 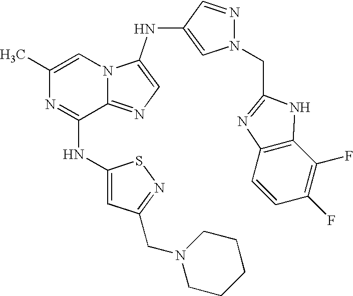 | 575.6 | | |
| 19-26 | 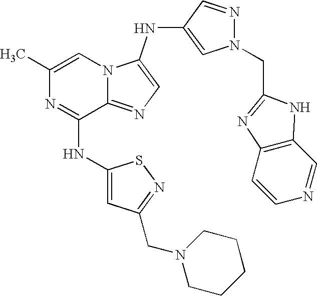 | 540.6 | | |

### Example 20

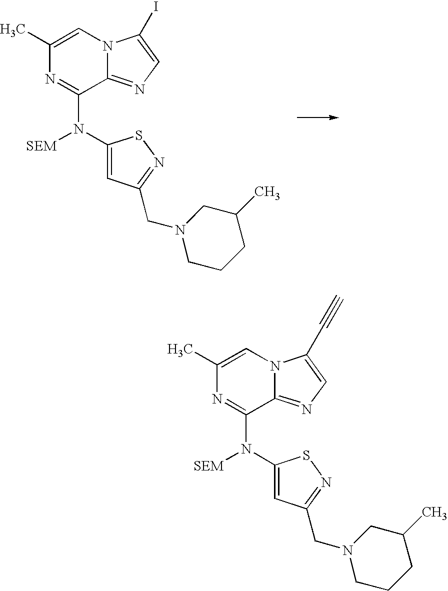

A solution of trimethylsilyl acetylene (46 mg, 0.46 mmol) in triethylamine (0.5 mL) was added to a nitrogen flushed mixture of iodide (136 mg, 0.23 mmol), palladium(0) triphenylphosphine (26 mg, 0.02 mmol) and copper(I) iodide (8.6 mg, 0.4 mmol). The reaction was stirred at room temperature for 12 hours then diluted with ethyl acetate (25 mL). The reaction contents were passed through Celite, concentrated, and placed onto a column (SiO₂; 12 g; 10% to 50% ethyl acetate in hexanes) which afforded the desired coupled intermediate. The intermediate was dissolved in methanol (8 mL) then treated with potassium carbonate (790 mg). The reaction was stirred at room temperature for 72 hours then diluted with methylene chloride (50 mL). The organic layer was washed with saturated aqueous sodium bicarbonate (50 mL), dried (sodium sulfate), filtered and concentrated to dryness. The resultant residue was placed onto a flash column (SiO₂; 4 g; 10% to 50% ethyl acetate in hexanes) to afford the title compound as a white solid 19 mg (17%). ¹H NMR (300 MHz, CDCl₃) δ 7.91 (s, 1H), 7.82 (s, 1H), 7.31 (s, 1H), 6.67 (s, 2H), 3.91 (s, 1H), 3.81 (t, J=6.4 Hz, 2H), 3.71 (s, 2H), 3.03-2.87 (m, 2H), 2.66 (s, 3H), 2.19-1.99 (m, 2H), 1.85-1.63 (m, 5H), 1.01 (t, J=6.4 Hz, 2H), 0.93 (d, J=5.6 Hz, 3H), 0.00 (s, 9H).

### Example 21

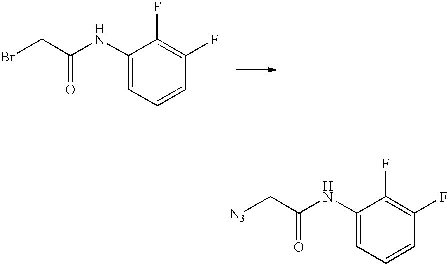

Sodium azide (116 mg, 1.79 mmol) was added to a solution of bromide (408 mg, 1.63 mmol) in dimethylformamide (30 mL). The reaction mixture was then heated to 60° C. and stirred for 12 hours. Upon completion, the reaction was cooled to room temperature and the solvent was removed in vacuo. The resultant residue was taken into ethyl acetate (75 mL) then washed with sodium bicarbonate (50 mL), water (50 mL) and brine (50 mL). The organic layer was then dried (sodium sulfate), filtered, and concentrated to dryness. The resultant solid was placed onto a flash column (SiO₂; 12 g; 10% to 50% ethyl acetate in hexanes) to give the title compound as a white solid 240 mg (69%). ¹H NMR (300 MHz, CDCl₃) δ 8.28 (br s, 1H), 8.12-7.96 (m, 1H), 7.16-7.02 (m, 1H), 7.01-6.87 (m, 1H), 4.20 (s, 2H).

### Example 22

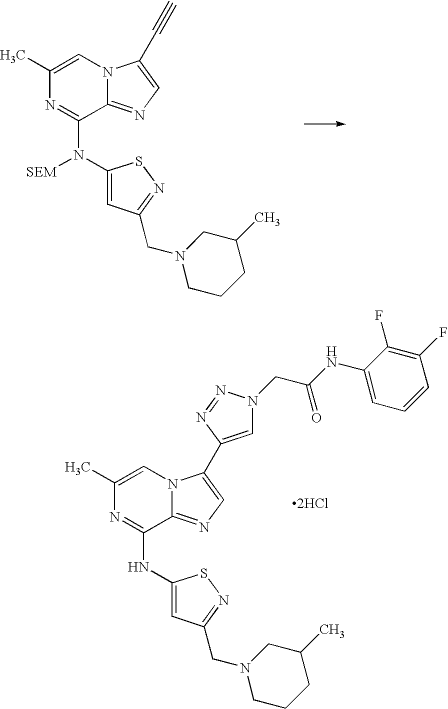

Copper powder (5 mg, 0.08 mmol) was added to a solution of alkyne (19 mg, 0.04 mmol) from Example 21 and azide (16 mg, 0.08 mmol) from Example 22 in t-butyl alcohol (0.3 mL) and water (0.6 mL). The reaction was stirred at room temperature for 72 hours then diluted with ethyl acetate (100 mL). The organic layer was washed with water (100 mL) and brine (100 mL) then dried (sodium sulfate), filtered and concentrated. The resultant residue was placed onto a flash column (SiO₂; 4 g; 0% to 10% methanol in methylene chloride) to afford the coupled intermediate. The desired intermediate was then dissolved in dioxane (2 mL) and treated with 4 N HCl in dioxane (2 mL). The reaction was sonicated at room temperature for 1 hour. The solvent was removed and the residue was purified by prep-HPLC (95:5 to 5:95 water/acetonitrile with 0.1% trifluoroacetic acid). The fractions were collected and dried and the residue treated with 0.2 N HCl and freeze-dried to afford the title compound as a white solid 6.9 mg (28%). NMR (300 MHz, CD₃OD) δ 8.65 (s, 1H), 8.58 (s, 1H), 8.13 (s, 1H), 7.77-7.70 (m, 1H), 7.21 (s, 1H), 7.17-7.04 (m, 2H), 5.59 (s, 2H), 4.38 (s, 2H), 3.71-3.46 (m, 2H), 3.13-3.09 (m, 1H), 2.79-2.63 (m, 1H), 2.58 (s, 3H), 2.06-1.74 (m, 4H), 1.32-1.11 (m, 1H), 1.00 (d, J=6.4 Hz, 3H). HPLC t_{R}=5.45 min (UV₂₅₄ₙₘ). Mass calculated for formula C₂₇H₂₈F₂N₁₀OS 578.2; observed MH⁺ (ESI MS) 579.8 (m/z).

### Example 23

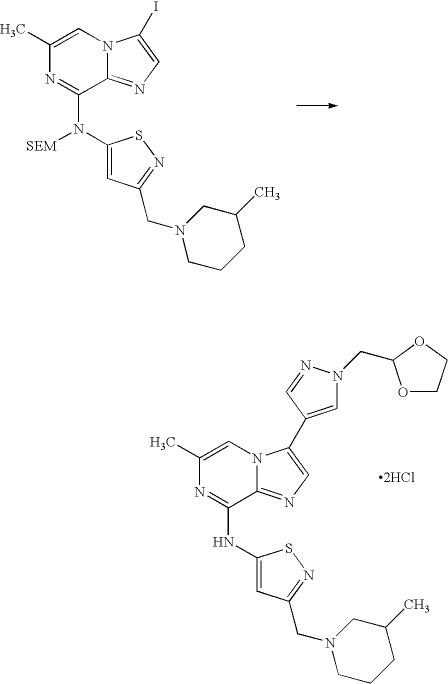

Example 24 was prepared in a similar manner to Example 15. ¹H NMR (300 MHz, CD₃OD) δ 8.30 (s, 1H), 8.21 (s, 1H), 8.03 (s, 1H), 8.00 (s, 1H), 7.35 (s, 1H), 5.31-5.22 (m, 1H), 4.49-4.37 (m, 4H), 3.91 (s, 4H), 3.69-3.46 (m, 2H), 3.10-2.91 (m, 1H), 2.80-2.66 (m, 1H), 2.63 (s, 3H), 2.07-1.74 (m. 4H), 1.39-1.12 (m, 1H), 1.00 (d, J=6.5 Hz, 3H). HPLC t_{R}=7.36 min (UV 254 nm). Mass calculated for formula C₂₄H₃₀N₈O₂S 494.2; observed MH⁺ (ESI MS) 495.8 (m/z).

### Example 24

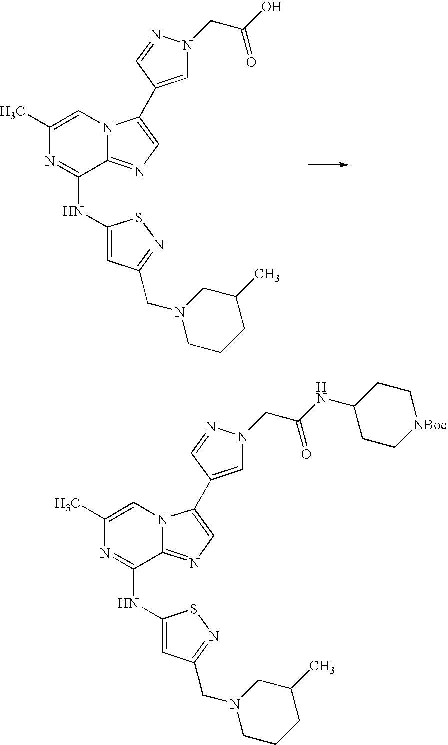

Example 25 was prepared in a similar manner to Example 6. ¹H NMR (300 MHz, CD₃OD) δ 8.20 (s, 1H), 7.94 (s, 1H), 7.85 (s, 1H), 7.77 (s, 1H), 7.18 (s, 1H), 4.98 (s, 2H), 4.37 (br s, 2H), 4.17-3.81 (m, 2H), 3.71-3.40 (m, 2H), 3.20-3.05 (m, 1H), 3.03-2.84 (m, 2H), 2.79-2.62 (m, 1H), 2.52 (s, 3H), 2.06-1.97 (m, 4H), 1.97-1.69 (m, 5H), 1.46 (s, 9H), 1.32-1.14 (m, 1H), 1.00 (d, J=6.4 Hz, 3H).

### Example 25

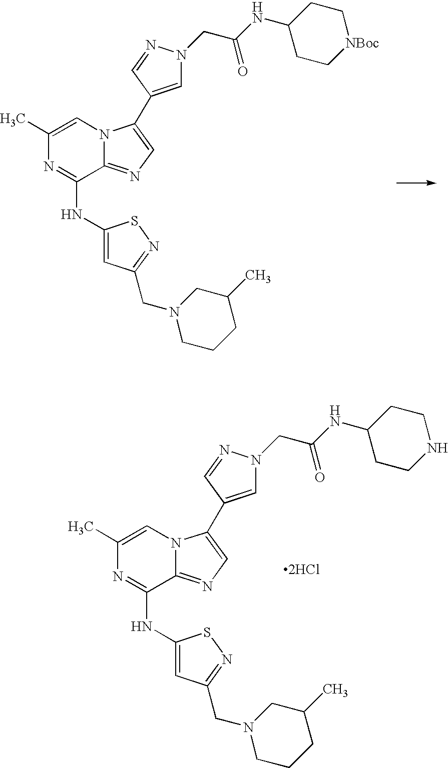

Trifluoroacetic acid (2 mL) was added to a solution of amide (20 mg, 0.02 mmol) in methylene chloride (2 mL). The reaction was allowed to stir at room temperature for 2 hours. The solvent was removed and the resultant residue placed onto a prep-HPLC (95:5 to 40:60 water/acetonitrile with 0.1% trifluoroacetic acid). The collected fractions were concentrated then treated with 0.2 N HCl and freeze-dried to afford the title compound as a white solid 3.2 mg (23%). ¹H NMR (300 MHz, CD₃OD) δ 8.23 (s, 1H), 7.96 (s, 1H), 7.91 (s, 1H), 7.85 (s, 1H), 7.21 (s, 1H), 5.00 (s, 2H), 4.39 (br s, 2H), 4.09-3.93 (m, 1H), 3.68-3.39 (m, 4H), 3.20-3.04 (m, 2H), 3.04-2.89 (m, 1H), 2.79-2.63 (m, 1H), 2.51 (s, 3H), 2.24-2.09 (m, 2H), 2.05-1.67 (m, 6H), 1.32-1.12 (m, 1H), 1.01 (d, J=6.4 Hz, 3H). HPLC t_{R}=3.47 min (UV₂₅₄ₙₘ). Mass calculated for formula C₂₇H₃₆H₁₀OS 548.3; observed MH⁺ (ESI MS) 549.9 (m/z).

### Example 26

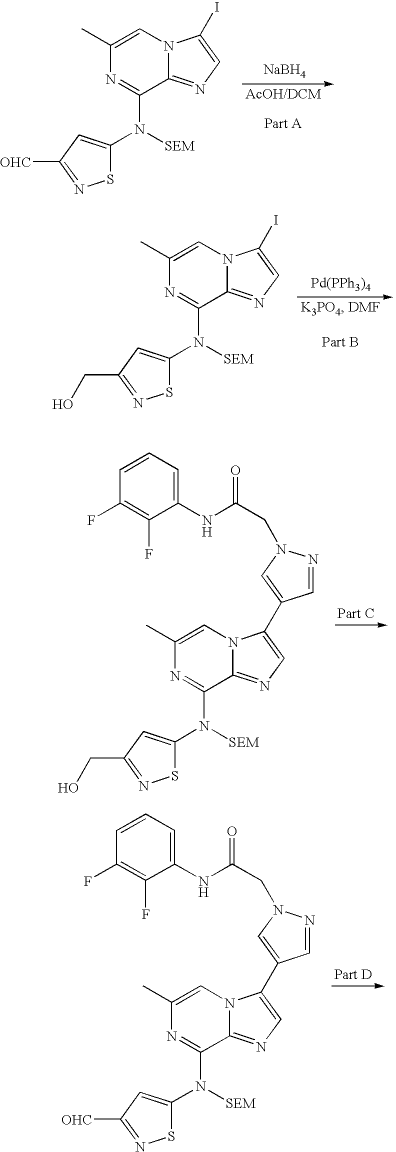

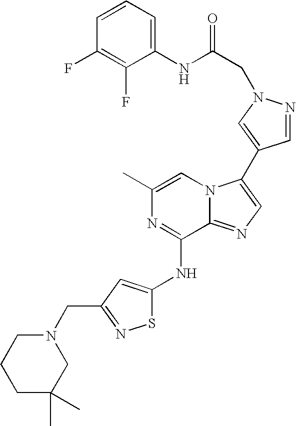

Part A: To a solution of iodide (390 mg, 0.757 mmol) in 10 mL of CH₂Cl₂ was added 8 mL of AcOH. NaBH₄ (57 mg, 1.51 mmol) was then added in one portion. The reaction was stirred at room temperature for 15 min. It was diluted with 100 mL of CH₂Cl₂, and neutralized by 5 N NaOH (aq.). To the mixture was added 100 ml of saturated aqueous NaHCO₃. The resulting mixture was stirred at room temperature for 30 min. The organic layer was isolated. It was dried over anhydrous Na₂SO₄, and then concentrated. The residue was purified by flash chromatography eluting with 60% EtOAc/CH₂Cl₂ to give 390 mg of the title compound. ¹H NMR (400 MHz, CDCl₃) δ 7.72 (s, 1H), 7.60 (s, 1H), 7.13 (s, 1H), 6.58 (brs, 2H), 4.78 (s, 2H), 3.72 (t, 2H), 2.60 (s, 3H), 0.95 (t, 2H), −0.10 (s, 9H).

Part B: To a mixture of alcohol from Part A (80 mg, 0.15 mmol), boronate from Example 7,

Part A (84 mg, 0.23 mmol) and Pd(PPh₃)₄ (17.8 mg, 0.015 mmol) was added 2 mL of DMF followed by 3 M aqueous K₃PO₄ solution (0.21 mL, 0.63 mmol). The reaction mixture was heated at 65° C. for 18 h. It was diluted with 30 mL of EtOAc and washed with 1 N aqueous NH₄Cl solution (20 mL×2). The organic layer was concentrated under vacuum. The residue was purified by flash chromatography eluting with 4% MeOH/CH₂Cl₂ to give 68 m g of 5. ¹H NMR (400 MHz, CDCl₃) δ 8.90 (brs, 1H), 8.02-8.12 (m, 1H), 8.00 (s, 1H), 7.87 (s, 1H), 7.68 (s, 1H), 7.57 (s, 1H), 7.15 (s, 1H), 6.90-7.13 (m, 2H), 6.65 (brs, 2H), 5.09 (s, 2H), 4.75-4.81 (m, 2H), 3.77 (t, 2H), 2.80 (brs, 1H), 2.60 (s, 3H), 0.95 (t, 2H), −0.10 (s, 9H).

Part C: To a solution of alcohol from Part B (31 mg, 0.049 mmol) in 1.5 mL of THF, was added 3 □L of water followed by Dess-Martin periodinane (64 mg, 0.15 mmol). The reaction mixture was stirred at room temperature for 30 min. It was diluted with 5 mL of THF. The mixture was filtered. The filtrate was diluted with 20 mL of CH₂Cl₂ and washed with 10 mL of saturated aqueous NaHCO₃ solution. It was dried over anhydrous Na₂SO₄ and then concentrated to give 30 mg of the title compound which was used in the subsequent reactions without further purification.

Part D: A solution of aldehyde (12 mg, 0.019 mmol), 3,3-dimethylpiperidine (22 mg, 0.19 mmol) in 1 mL of CH₂Cl₂ was stirred at room temperature for 30 min. To the solution was added NaBH₄ (3.6 mg, 0.096 mmol) followed by 0.3 mL of MeOH. The reaction was stirred at room temperature for 1 h. It was diluted with 10 mL of CH₂Cl₂ and 10 mL of saturated aqueous NaHCO₃ solution. The resulting mixture was stirred for 1 h. The organic was separated and concentrated under vacuum. The residue was purified by flash chromatography eluting with NH₄OH (aq.)/MeOH/CH₂Cl₂ (1:10:190) to give 10 mg of the SEM-protected title compound. To a solution of SEM-protected material (10 mg, 0.014 mmol) in 2 mL of THF heated at 80° C. was added 0.2 mL of 4 N HCl in dioxane. The reaction was stirred at 80° C. for 1.5 h. It was cooled to room temperature and then added 8 mL of ether. The solid was collected by filtration and washed with ether to give 8.7 mg of the title compound. ¹H NMR (400 MHz, CD₃OD) δ 8.38 (s, 1H), 8.15 (s, 1H), 8.08 (s, 1H), 8.02 (s, 1H), 7.72-7.81 (m, 1H), 7.27 (s, 1H), 7.03-7.20 (m, 2H), 5.30 (s, 2H), 4.35-4.60 (m, 2H), 3.55-3.65 (d, 1H), 3.00-3.10 (m, 1H), 2.80-2.90 (d, 1H), 2.60 (s, 3H), 1.81-2.10 (m, 2H), 1.40-1.69 (m, 2H), 1.20 (s, 3H), 1.00 (s, 3H). HPLC-MS t_{R}=2.96 min (UV₂₅₄ₙₘ). Mass calculated for formula C₂₉H₃₁F₂N₉OS 591.2; observed MH⁺ (LCMS) 592.3 (m/z).

### Example 27

By essentially the same procedure set forth in Example 26, only replacing 3,3-dimethylpiperidine with other respective amines in Part A, compounds shown in column 2 of Table 9 (shown immediately below) were prepared.

TABLE 9

| | | | MS | |
|---|---|---|---|---|
| | | | MH⁺ | HPLC |
| Example | Column 2 | MW | m/z | t_{R} |
| 9-1 | 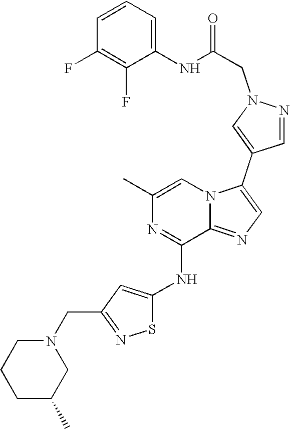 | 577.2 | 578.3 | 2.87 |
| 9-2 | 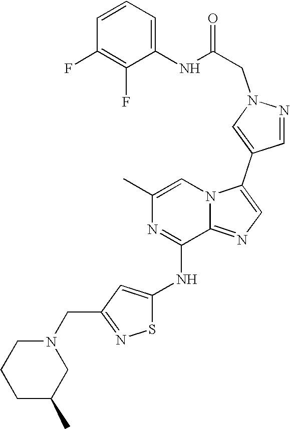 | 577.2 | 578.3 | 2.87 |

### Example 28

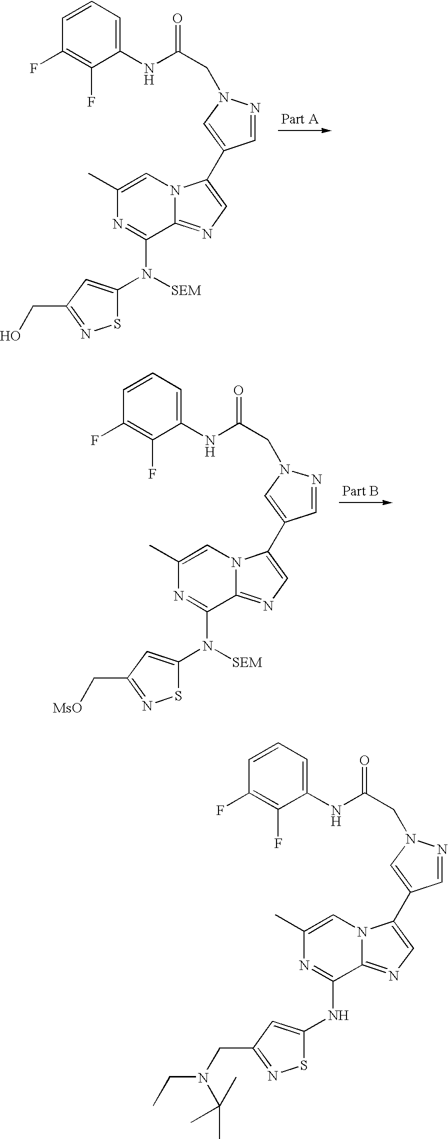

Part A: To a solution of alcohol from Example 26, Part B (30 mg, 0.047 mmol) in 1.5 mL of THF, was added triethylamine (9.5 mg, 0.094 mmol) followed by methanesulfonylchloride (7.3 □L, 0.094 mmol). The reaction was stirred at room temperature for 20 min. It was diluted with 10 mL of CH₂Cl₂, washed with 5 mL of 1 N aqueous HCl. The organic was dried over anhydrous Na₂SO₄. The solvent was removed to give 31 mg of the title compound as a crude material which was used in the subsequent reaction without further purification.

Part B: A mixture of mesylate from Part A (9.6 mg, 0.014 mmol), N,N-diethylisopropylamine (6.0 mg, 0.068 mmol) and NaI (4.1 mg, 0.027 mmol) in 1 mL of THF was stirred at 60° C. for 3 h. It was diluted with 10 mL of CH₂Cl₂ and washed with water. The organic was concentrated under vacuum. The residue was purified by flash chromatography eluting with NH₄OH (aq.)/MeOH/CH₂Cl₂ (1:10:190) to give 8 mg of N-(2,3-difluoro-phenyl)-2-(4-{8-[{3-[(ethyl-isopropyl-amino)-methyl]-isothiazol-5-yl}-(2-trimethylsilanyl-ethoxymethyl-amino]-6-methyl-imidazo[1,2-a]pyrazin-3-yl}-pyrazol-1-yl)-acetamide. To a solution of N-(2,3-difluoro-phenyl)-2-(4-{8-[{3-[(ethyl-isopropyl-amino)-methyl]-isothiazol-5-yl}-(2-trimethylsilanyl-ethoxymethyl)-amino]-6-methyl-imidazo[1,2-a]pyrazin-3-yl}-pyrazol-1-yl)-acetamide (8.0 mg, 0.011 mmol) in 2 mL of THF heated at 80° C. was added 0.2 mL of 4 N HCl in dioxane. The reaction was stirred at 80° C. for 1.5 h. It was cooled to room temperature and then added 8 mL of ether. The solid was collected by filtration and washed with ether to give 5.8 mg of the title compound. ¹H NMR (400 MHz, CD₃OD) δ 8.35 (s, 1H), 7.95-8.10 (m, 3H), 7.70-7.82 (m, 1H), 7.25 (s, 1H), 7.00-7.20 (m, 2H), 5.30 (s, 2H), 4.30-4.60 (m, 2H), 3.75-3.85 (m, 1H), 2.60 (s, 3H), 1.30-1.50 (m, 9H). HPLC-MS t_{R}=2.80 min (UV₂₅₄ₙₘ). Mass calculated for formula C₂₇H₂₉F₂N₉OS 565.2; observed MH⁺ (LCMS) 566.3 (m/z).

### Example 29

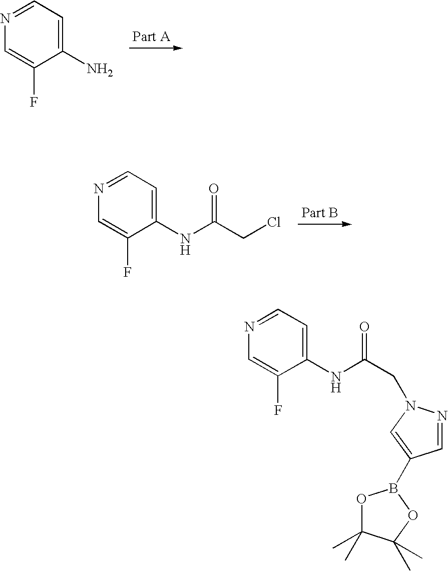

Part A: To a solution of 4-amino-3-fluoro pyridine (560 mg, 5.0 mmol) and Et₃N (760 mg, 7.5 mmol) in 20 mL of THF, was added chloroacetyl chloride (622 mg, 5.5 mmol). The reaction was stirred at room temperature and monitored by thin layer chromatography. More chloroacetyl chloride was added until 4-amino-3-fluoro pyridine was consumed. It was quenched by adding 20 mL of saturated aqueous NaHCO₃. The mixture was diluted with 150 mL of CH₂Cl₂. The organic layer was concentrated and purified by flash chromatography eluting with 35% EtOAc/CH₂Cl₂ to give 850 mg of the title compound. NMR (400 MHz, CDCl₃) δ 8.62 (brs, 1H), 8.41 (d, 1H), 8.33 (d, 1H), 8.26 (t, 1H), 4.20 (s, 2H).

Part B: A mixture of amide from Part A (106 mg, 0.55 mmol) and Cs₂CO₃ (326 mg, 1.0 mmol) in 2 mL of DMSO was heated at 100° C. for 5 min. To the mixture was added 4-pyrazoleboronic acid pinacol ester (94 mg, 0.50 mmol). The reaction was stirred at 100° C. for 20 min. It was cooled to room temperature and diluted with 30 mL of CH₂Cl₂. The mixture was washed with water. The organic was concentrated and purified by running a quick column eluting with 2% MeOH/EtOAc to give 52 mg of the title compound. NMR (400 MHz, CDCl₃) δ 9.30 (brs, 1H), 8.33 (d, 1H), 8.20-8.30 (m, 2H), 7.93 (s, 1H), 7.75 (s, 1H), 4.90 (s, 2H), 1.24 (s, 12H).

### Example 30

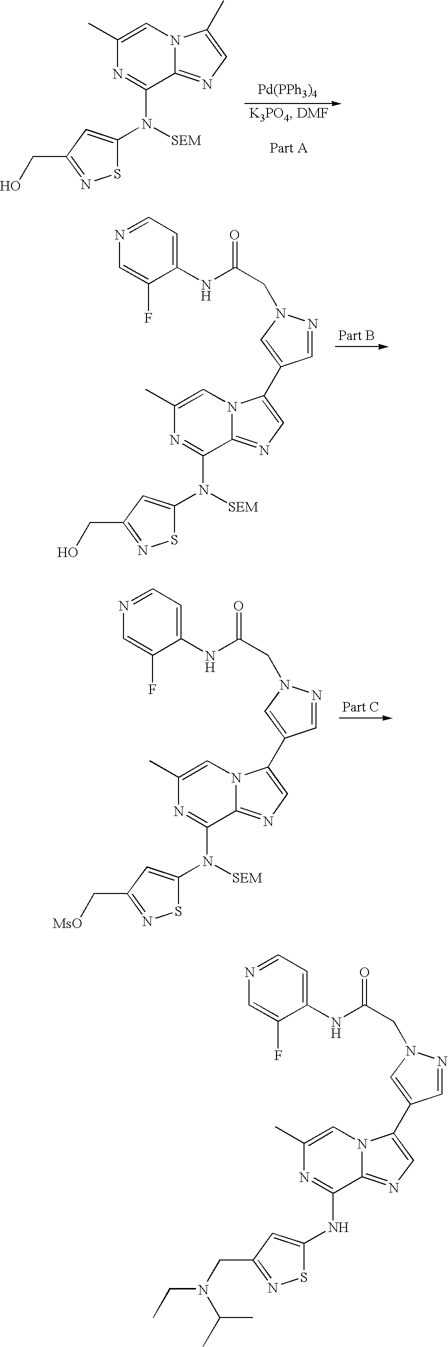

Part A: To a mixture of iodide (43 mg, 0.083 mmol), boronate from Example 29, Part B (43 mg, 0.124 mmol) and Pd(PPh₃)₄ (14 mg, 0.012 mmol) in a vial, was added 1.1 mL of DMF, followed by adding 0.11 mL of 3 M aqueous K₃PO₄ solution (0.33 mmol). The vial was sealed and stirred at 65° C. overnight. It was diluted with 30 mL of EtOAc and washed with water. It was concentrated and purified by flash chromatography eluting with 7% MeOH/DCM to give 24 mg of the title compound. NMR (400 MHz, CDCl₃) δ 9.25 (brs, 1H), 8.25-8.43 (m, 3H), 7.95 (s, 1H), 7.80 (s, 1H), 7.61 (s, 1H), 7.50 (s, 1H), 7.09 (s, 1H), 6.60 (s, 2H), 5.05 (s, 2H), 4.72 (s, 2H), 3.70 (t, 2H), 2.75 (brs, 1H), 2.48 (s, 3H), 0.90 (t, 2H), −0.14 (s, 9H).

Part B: To a solution of alcohol from Part A (102 mg, 0.17 mmol) in 5 mL of THF, was added NEt₃ (0.094 mL, 0.67 mmol), followed by methanesulfonylchloride (0.029 mL, 0.37 mmol). The reaction was stirred at room temperature for 15 min. It was monitored by thin layer chromatography and found starting alcohol was not totally consumed. Additional methanesulfonylchloride (0.0035 mL, 0.039 mmol) was added. The stirring was continued for 5 min. It was quenched by adding 2 mL of saturated NH₄Cl (aq.) and 2 mL of water. The organic layer was collected. The aqueous layer was extracted with CH₂Cl₂ (10 mL×3) until no desired product remains in aqueous layer. The combined organics were further purified by flash chromatography eluting with MeOH/CH₂Cl₂ (1:10) to give 63.3 mg of the title compound as a light yellow solid. ¹H NMR (400 MHz, DMSO-d₆) δ 10.75 (s, 1H), 8.60 (d, 1H), 8.51 (s, 1H), 8.36 (d, 1H), 8.20 (t, 1H), 8.10 (s, 2H), 7.96 (s, 1H), 7.38 (s, 1H), 6.70 (brs, 2H), 5.34 (s, 2H), 5.28 (s, 2H), 3.68 (t, 2H), 3.28 (s, 3H), 2.54 (s, 3H), 0.85 (t, 2H), −0.10 (s, 9H).

Part C: A mixture of mesylate from Part B (24.7 mg, 0.036 mmol), N,N-diethylisopropylamine (7.8 mg, 0.089 mmol) and NaI (1 mg, 0.007 mmol) in 1.5 mL of THF was stirred at 80° C. for 4 h. It was diluted with 10 mL of CH₂Cl₂ and washed with water and brine. It was dried over anhydrous Na₂SO₄. The organic was concentrated under vacuum. The residue was purified by flash chromatography eluting with 7N NH₃ in MeOH/CH₂Cl₂ (1:30) to give 11.5 mg of 2-(4-{8-[{3-[(ethyl-isopropyl-amino)-methyl]-isothiazol-5-yl}-(2-trimethylsilanyl-ethoxymethyl)-amino]-6-methyl-imidazo[1,2-a]pyrazin-3-yl}-pyrazol-1-yl)-N-(3-fluoro-pyridin-4-yl)-acetamide. NMR (400 MHz, CDCl₃) δ 9.36 (brs, 1H), 8.30-8.48 (m, 3H), 8.02 (s, 1H), 7.85 (s, 1H), 7.66 (s, 1H), 7.55 (s, 1H), 7.30 (s, 1H), 6.62 (s, 2H), 3.60-3.85 (m, 4H), 2.96-3.15 (brs, 3H), 2.40-2.68 (m, 5H), 0.90-1.20 (m, 12H), 0.00 (s, 9H). To a solution of 2-(4-{8-[{3-[(ethyl-isopropyl-amino)-methyl]-isothiazol-5-yl}-(2-trimethylsilanyl-ethoxymethyl)-amino]-6-methyl-imidazo[1,2-a]pyrazin-3-yl}-pyrazol-1-yl)-N-(3-fluoro-pyridin-4-yl)-acetamide (11.5 mg, 0.0169 mmol) in 0.4 mL of THF heated at 80° C. was added 0.4 mL of 4 N HCl in dioxane. The reaction was stirred at 80° C. for 1 h. It was cooled to room temperature and then added 8 mL of ether. The solid was collected by filtration and washed with ether to give 10 mg of the title compound. HPLC-MS t_{R}=2.18 min (UV₂₅₄ₙₘ.) Mass calculated for formula C₂₆H₂₉FN₁₀OS 548.2; observed MH⁺ (LCMS) 549.3 (m/z).

### Example 31

By essentially the same procedure set forth in Example 30, only replacing ethylisopropylamine with other respective amines in Part C, compounds shown in column 2 of Table 10 (shown immediately below) were prepared.

TABLE 10

| | | | MS | |
|---|---|---|---|---|
| | | | MH⁺ | HPLC |
| Example | Column 2 | MW | m/z | t_{R} |
| 17-1 | 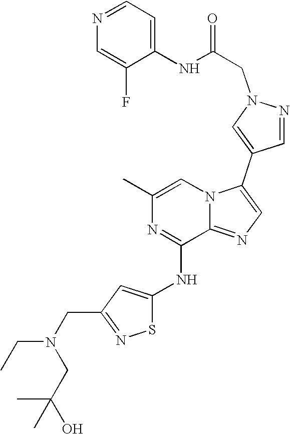 | 578.7 | 579.3 | 2.17 |
| 17-2 | 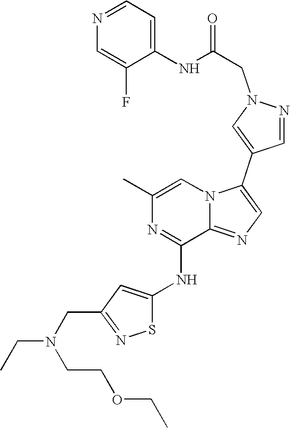 | 578.7 | 579.3 | 2.28 |
| 17-3 | 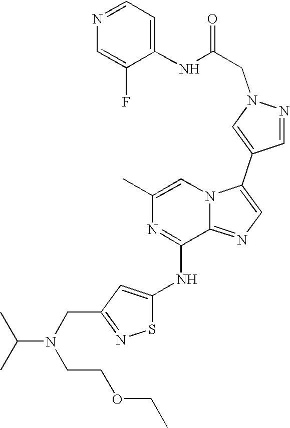 | 592.7 | 593.3 | 2.23 |
| 17-4 | 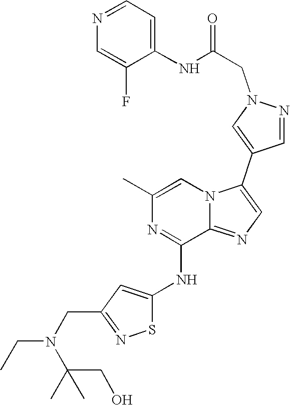 | 578.7 | 579.3 | 2.28 |
| 17-5 | 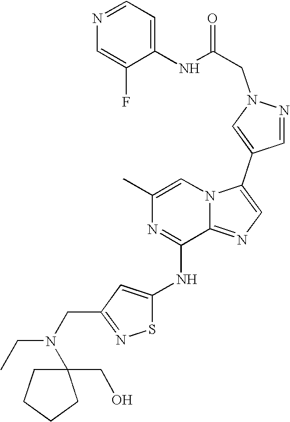 | 604.7 | 605.3 | 2.40 |

### EXAMPLES

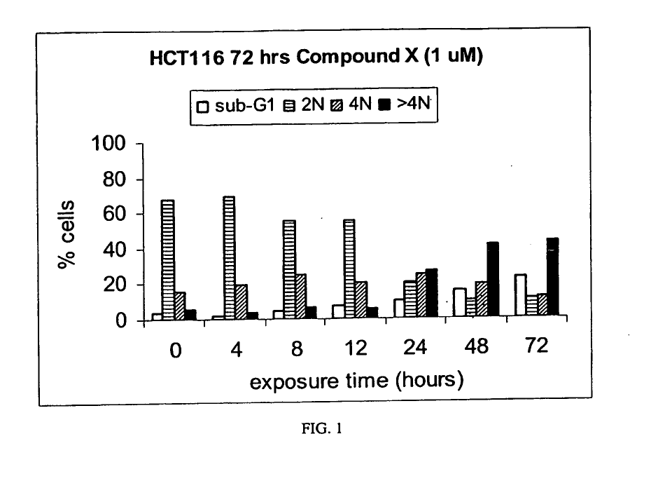

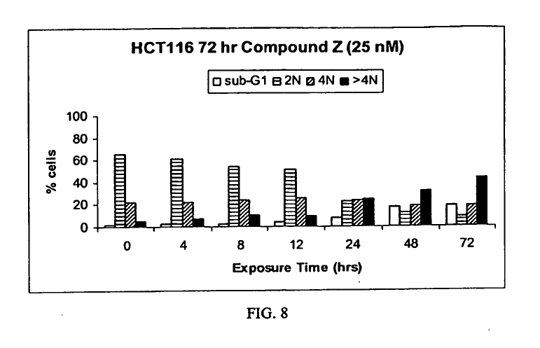

Asynchronous cells require 24 hour exposure to Aurora kinase inhibitors (such as 250 nM VX-680 or 1000 nM COMPOUND X. (Compound X is disclosed in WO 2008/057512, filed Nov. 6, 2007, Example 4-3 and Claim 70, Example 4-3 and Claim 70 and is shown below and it is represented in Formula H and Table 2, column 2, row 17 of the present application) or 25 nM COMPOUND Z (Compound Z is disclosed in PCT US2008/007295, filed Jun. 11, 2008, Example 76-2 and Claim 25, row 7, column 4 and it is represented in Table 13, compound 76-2 of the present application)) to induce endoreduplication (>4N DNA content) or cell death. For example, HCT-116 colon cancer cells were treated with 1000 nM COMPOUND X or 25 nM COMPOUND Z for the indicated times at which time the drug was washed off and replaced with new media see FIG. 1 and FIG. 8 respectively. Cells were analyzed by FACS after a total of 72 hours. Exposures less than 24 hours were insufficient to induce endoreduplication (>4N DNA content), while drug exposure for 24, 48, or 72 hours resulted in the accumulation of cells that underwent endoreduplication. Similar findings were found in HT29 colon, MCF-7 and T47D breast, NCI-460 and A549 lung, PC3, DU145, LNCAp prostate, A2780, SKOV3, PA1, TOV112D, and ES2 ovarian cells.

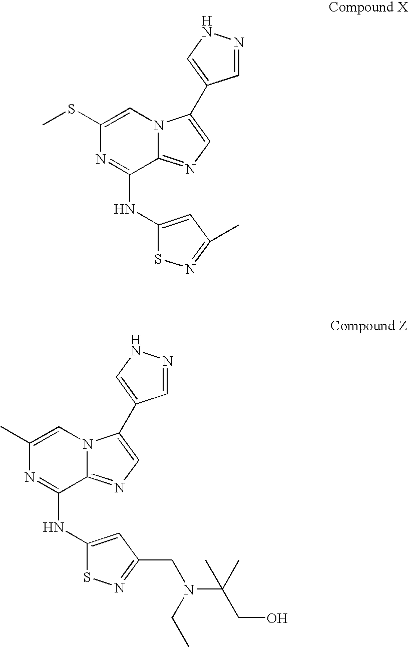

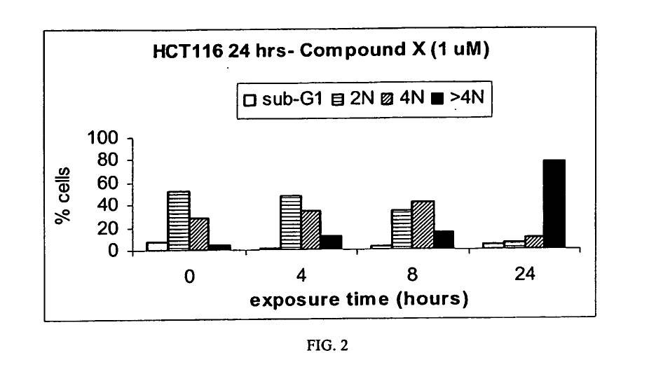

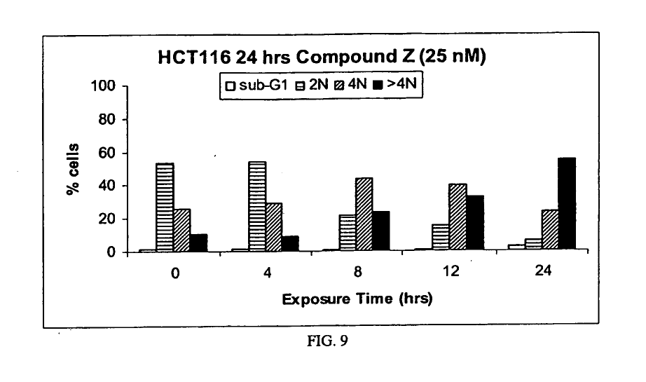

When asynchronous cells were analyzed by FACS at 24 hours, 24 hour exposure to an Aurora kinase inhibitor (such as 250 nM VX-680 or 1000 nM COMPOUND X) was sufficient to induce endoreduplication. However less exposure time was insufficient to induce endoreduplication. For example, HCT-116 colon cancer cells were treated with 1000 nM COMPOUND X or 25 nM COMPOUND Z for the indicated times at which time the drug was washed off and replaced with new media see FIG. 2 and FIG. 9 respectively. Cells were analyzed by FACS after a total of 24 hours. Exposures less than 24 hours were insufficient to induce endoreduplication (>4N DNA content), while drug exposure for the entire 24 hours resulted in the accumulation of cells that underwent endoreduplication.

Interestingly, following a 16 hour pre-treatment with taxanes (Taxol or Taxotere) or KSP inhibitors, Ispinesib SB-715992 or COMPOUND A (Compound A is a KSP inhibitor disclosed in WO 2006/098,961 filed Mar. 7, 2006, Example 263, Claim 51 and Compound A, shown below, is represented in the present application in Formula B and Table 1, column 2, row 7). Following the pre-treatment of the HCT-116 colon cancer cells with a taxane or the KSP inhibitor described above, the time of exposure to the Aurora kinase inhibitor, Compound X or Z required to induce endoreduplication, was reduced to less than 4-hours.

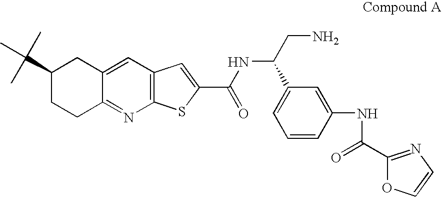

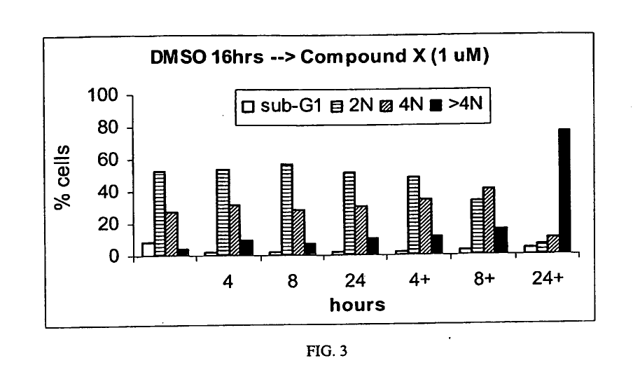

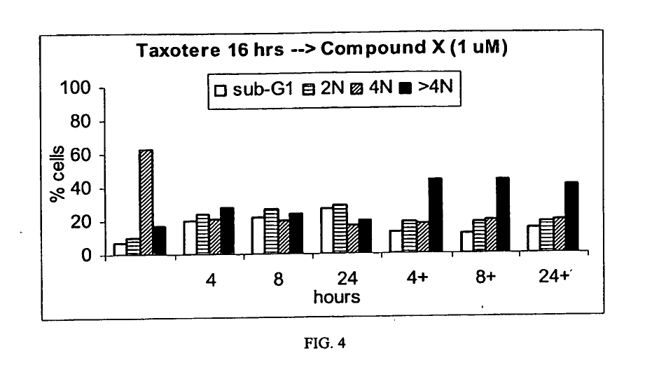

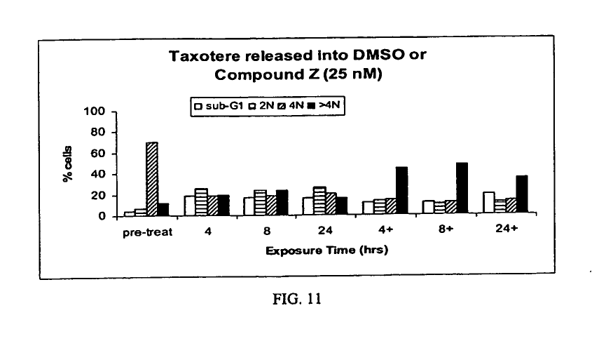

For example, HCT-116 colon cancer cells were treated for 16 hours with DMSO (FIG. 3) or 5 nM taxotere and then exposed to DMSO or 1000 nM COMPOUND X or 25 nM COMPOUND Z for 4, 8, and 24 hours. After 4 or 8 hours, COMPOUND X or COMPOUND Z was washed off and new media was added to the cells. Cells were harvested after 24 hours and analyzed by FACS (see FIG. 4 and FIG. 11 respectively). Following DMSO pretreatment, DMSO had no effect on cell cycle distribution. DMSO pretreatment followed by COMPOUND X or COMPOUND Z induced endoreduplication, but only when COMPOUND X or COMPOUND Z exposure was 24 hours, similar to the results in FIG. 2. Taxotere pretreatment (labeled 0 hours) cells accumulated in mitosis (4N DNA). Over time, when released into DMSO the cells exited mitosis. Taxotere followed by COMPOUND X or COMPOUND Z induced endoreduplication. Endoreduplication was observed even when COMPOUND X or COMPOUND Z exposure was as little as 4 hours. A similar shortening of the time required for an Aurora kinase inhibitor to be exposed to cells in order to induce endoreduplication was observed following pretreatment with 5 nM taxol or 10-50 nM KSP inhibitors.

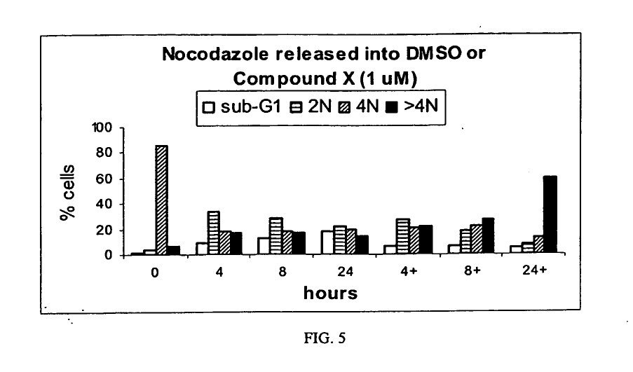

However, following a 16 hour pre-treatment with nocodazole for incrusting, the time required for an Aurora inhibitor (such as 250 nM VX-680 or 1000 nM COMPOUND X or COMPOUND Z) to induce endoreduplication was not reduced. For example, HCT-116 colon cancer cells were treated for 16 hours with 0.4 μg/ml nocodazole see FIG. 5, and then exposed to DMSO or 1000 nM COMPOUND X or COMPOUND Z for 4, 8 or 24 hours. After 4 or 8 hours, COMPOUND X or COMPOUND Z was washed off and new media was added to the cells. Cells were harvested after 24 hours and analyzed by FACS. Following nocodazole pretreatment (labeled 0 hour) cells accumulated in mitosis (4N DNA). Over time, when released into DMSO the cells exited mitosis. Nocodazole followed by 24 hour exposure to COMPOUND X or COMPOUND Z induced endoreduplication, however a 4- or 8-hour exposure to COMPOUND X or COMPOUND Z was insufficient to induce endoreduplication.

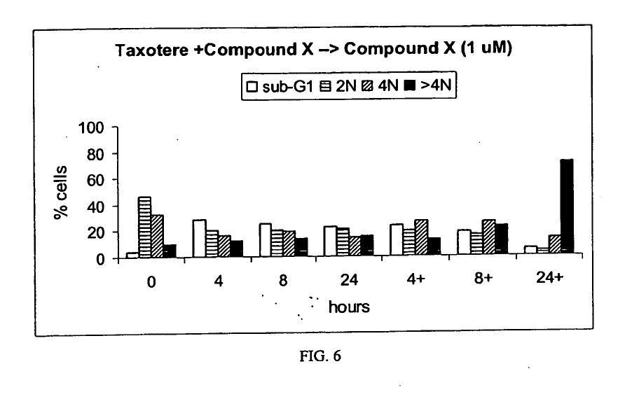

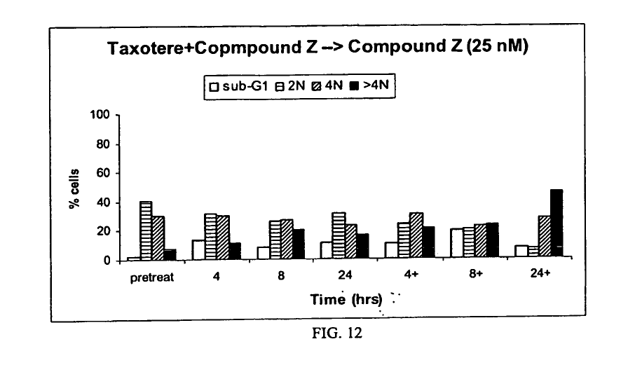

To further explore the combination with taxotere we analyzed simultaneous treatment with an Aurora kinase inhibitor, Compound X or COMPOUND Z. If the Aurora kinase inhibitor is given at the same time as taxotere, 4 hour exposure is not sufficient and 24 hour exposure is needed to induce endoreduplication. For example, HCT-116 colon cancer cells that were not pretreated, were treated with 5 nM taxotere plus DMSO or 1000 nM COMPOUND X or COMPOUND Z concurrently for 4, 8, or 24 hours. At the indicated time the media was removed and replaced with new media and taxotere was added back into these cells. Cells were harvested after 24 hours and analyzed by FACS see FIG. 6 or FIG. 12 respectively. This data suggest that concurrent treatment was not sufficient to reduce the exposure time required for an Aurora kinase inhibitor to induce endoreduplication.

Single agent Aurora kinase inhibitors require a drug exposure time of 24 hours to induce endoreduplication in asynchronous cells. A 16 hour pre-treatment with a taxane or KSP inhibitor was sufficient to reduce the time required for an Aurora kinase inhibitor to induce endoreduplication but pre-treatment with other anti-mitotic agents tested (nocodazole and vincristine) did not result in the same reduction in required exposure time. Aurora kinase inhibitors (such as VX-680 and COMPOUND X and COMPOUND Z) are capable of inhibiting phosphorylation of Histone H3 (at serine-10) stimulated by taxanes, KSP inhibitor, and nocodazole. Therefore, this does not explain the difference. We found that Aurora kinase inhibitors accelerated the exit from mitosis, as measured by phos-MPM2 marker, in response to taxane and KSP induced arrest, but not that of a nocodazole arrest. This effect was seen with dual Aurora A/B inhibitors such as VX-680, COMPOUND X, COMPOUND Z and AT9283, as well as with the Aurora B selective inhibitor AZD1152.

Acceleration from taxane, but not nocodazole, confirms published reports of similar findings. Morrow et al. (Journal of Cell Science 2005; 118: 3639) and Ditchfield et. al. (Journal of Cell Biology 2003; 161: 267) showed the Aurora kinase inhibitor ZM4477439 abrogated mitotic arrest induced by taxol but not by nocodazole. The former group suggested this was caused by accelerated exit from taxol induced arrest. Hauf et al (Journal of Cell Biology 2003; 161: 281) demonstrated that Hesperadin, a non-selective Aurora kinase inhibitor, caused cells arrested with taxol or monastrol (an inhibitor of KSP) to enter anaphase in less than 1 hour, whereas cells in nocodazole stay arrested for 3-5 hours. Similarly, Carvalho et al (Journal of Cell Science 2003; 116: 2987) showed cells depleted of Surviving (a regulator of Aurora B) by siRNA arrested in mitosis in the presence of nocodazole but not Taxol.

Our data suggests clinically, shorter exposures of cancer cells to Aurora kinase inhibitors may be needed if it follows taxane treatment. Ideal treatment of cancer cells with an Aurora kinase inhibitor would follow treatment of the cancer cells with anti-mitotic agents such as taxanes.

The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

What is claimed is:

1. A pharmaceutical composition for treating or ameliorating cancer comprising in combination (a) at least one anti-mitotic agent selected from the group consisting of a taxane, paclitaxel, docetaxel, a Cenp-E Inhibitor, Abraxane, Epothilone, Monastrol, a KSP inhibitor, Ispinesib, and a compound of Formulas A-D Formulas A-D shown in paragraphs a-d below:
a. A compound represented by the structural Formula A: 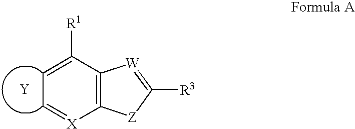
or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴R⁵, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰C(O)OR¹¹, and —NR¹⁰C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
b. A compound represented by the structural Formula B: 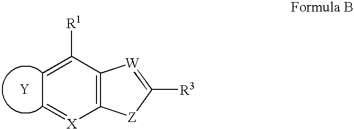
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)R⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R)(R⁷)₁₋₅—C(O)—NR⁴R⁵,
—C(O)N(R⁷)(CR⁴⁰R⁴¹)₁₋₅—OR⁷, —C(S)NR⁷(CH₂)₁₋₅NR⁴R⁵, and
—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,
—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰,
—CN, —NR¹⁰R¹¹, —C(O)OR¹⁰, —C(O)NR¹⁰R¹¹, —CF₃, —OCF₃,
—N(R¹⁰)C(O)R¹¹, and —NR¹⁰)C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹), —C(O)OR¹¹, —C(O)N(R¹⁰ (R¹¹), or —N(R¹⁰)C(O)R¹¹;
c. 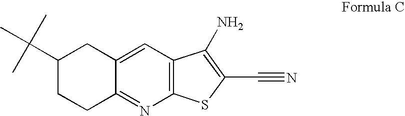
or a pharmaceutically acceptable salt, solvate, or ester thereof; and
d. A compound of Formula D 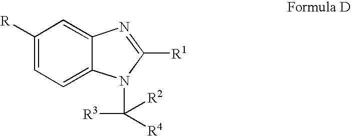
or a pharmaceutically acceptable salt, solvate, or ester thereof; wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(O)₂heterocyclyl, —S(═O)₂heteroaryl, hydroxy, alkyl, alkenyl, alkynyl, —NH₂, —NH(alkyl), —N(alkyl), alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
and (b) one or more of a second compound, wherein said second compound is an aurora kinase inhibitor selected from the group consisting of compounds represented by Formulas E-K shown below in paragraphs e-k:
e. A compound represented by the structural Formula E: 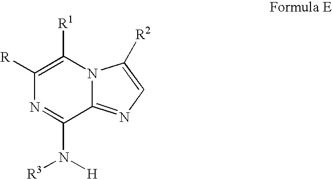
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
—N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
—NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
—CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
—S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring;
f. A compound represented by the structural formula: 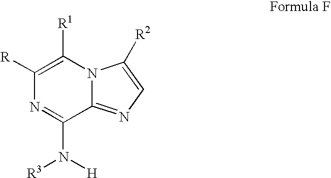
or a pharmaceutically acceptable salt, solvate, ester or prodrug thereof, wherein:
R is selected from the group consisting of H, halogen, aryl, heteroaryl, cycloalkyl, arylalkyl, heterocyclyl, heterocyclylalkyl, alkenyl, alkynyl,
—C(O)R⁷, 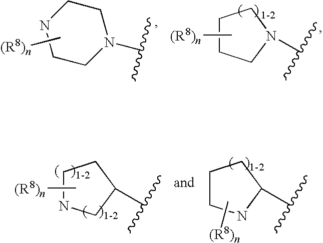
wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R¹ is H, halogen or alkyl;
R² is alkyl;
R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₙ—NR⁸, —(CHR⁵)ₙ—CH(aryl)₂, 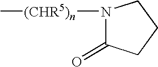
and 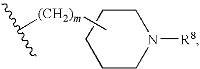
wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H or alkyl;
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and
—N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,
—S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4;
n is 1-4; and
p is 0-3;
g. A compound selected from the compounds of the formulas: 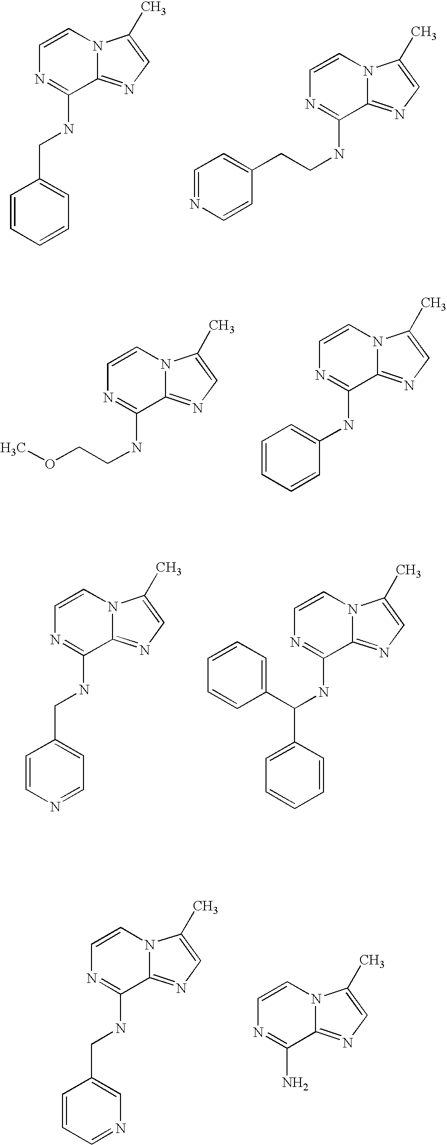 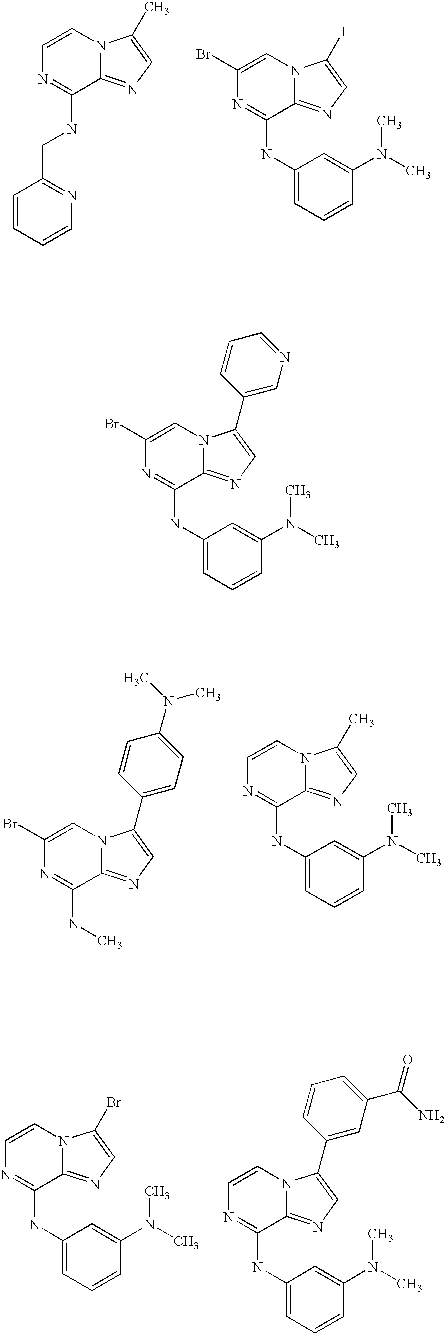 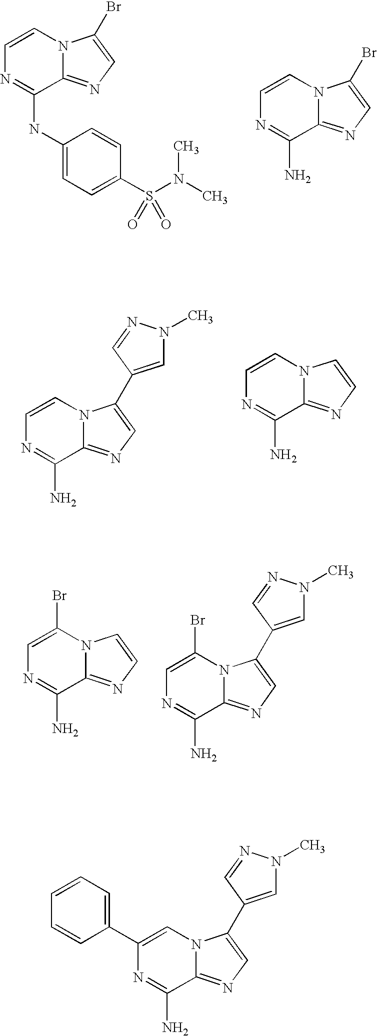 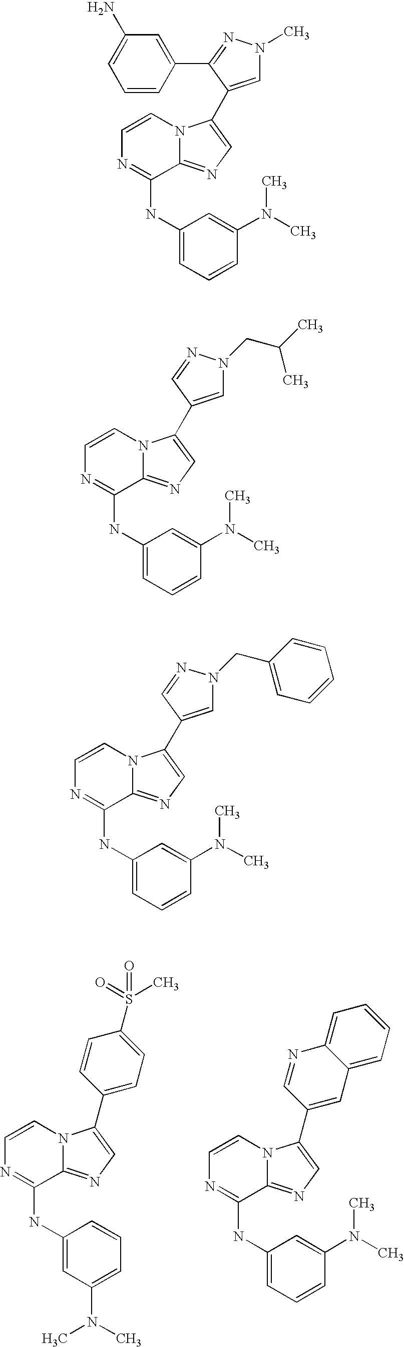 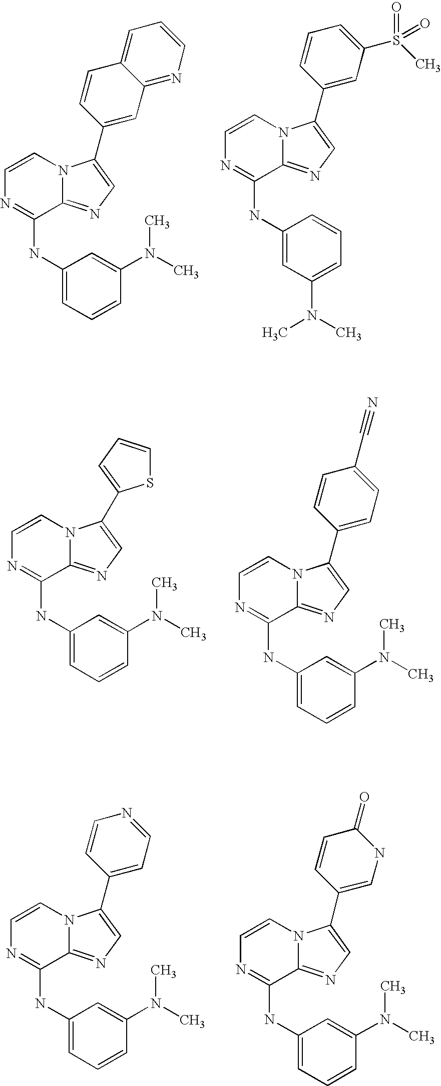 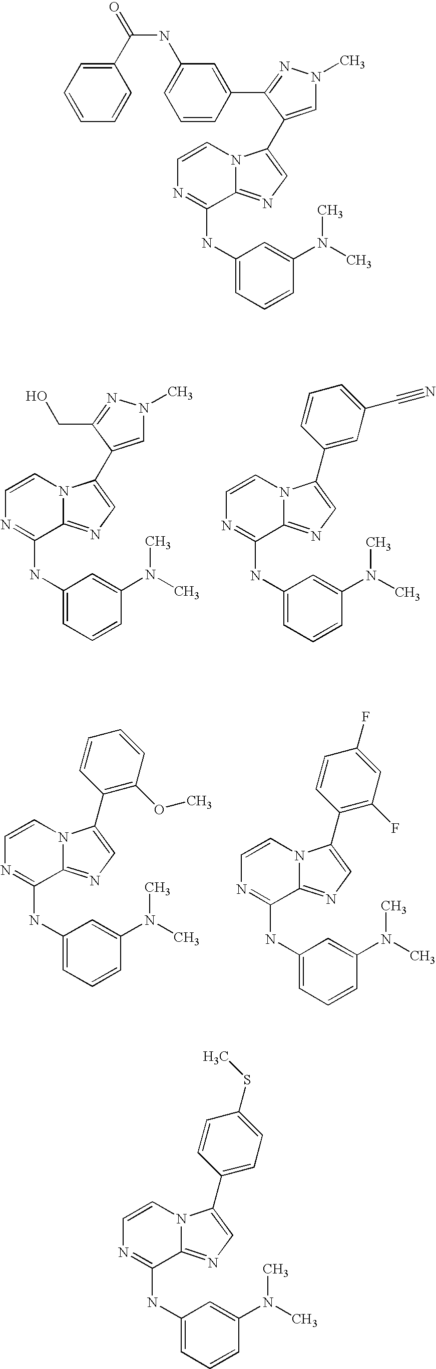 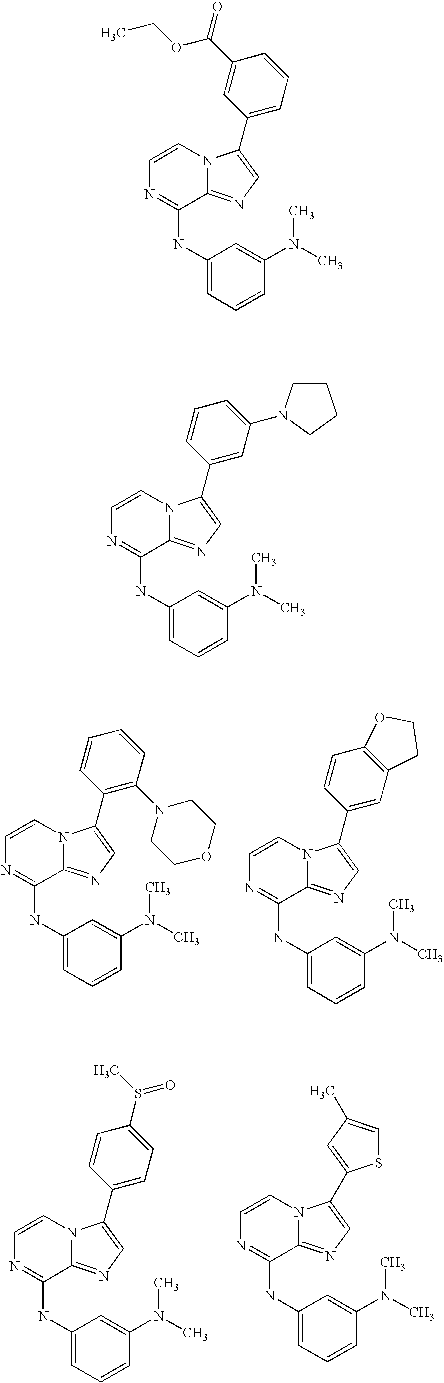 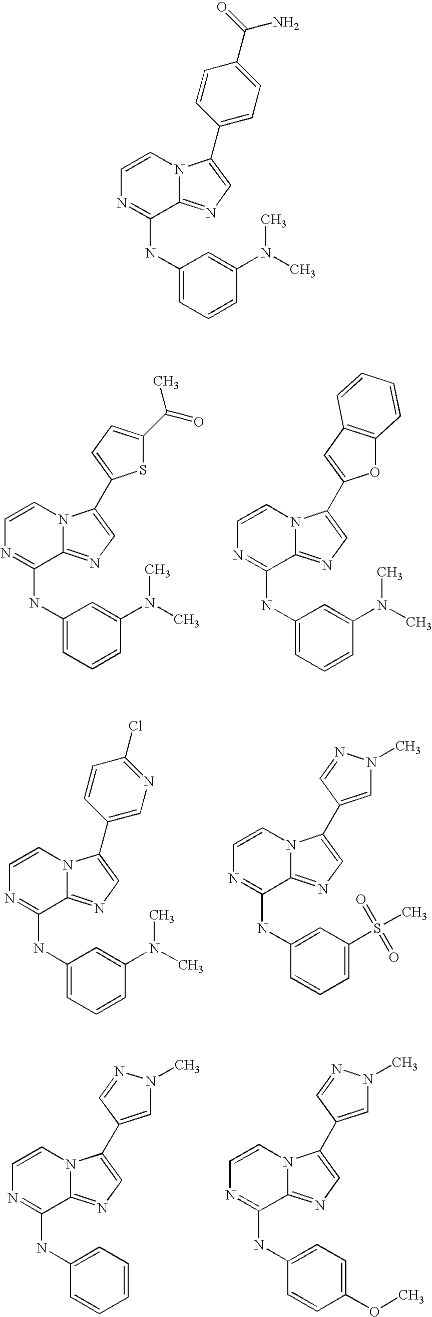 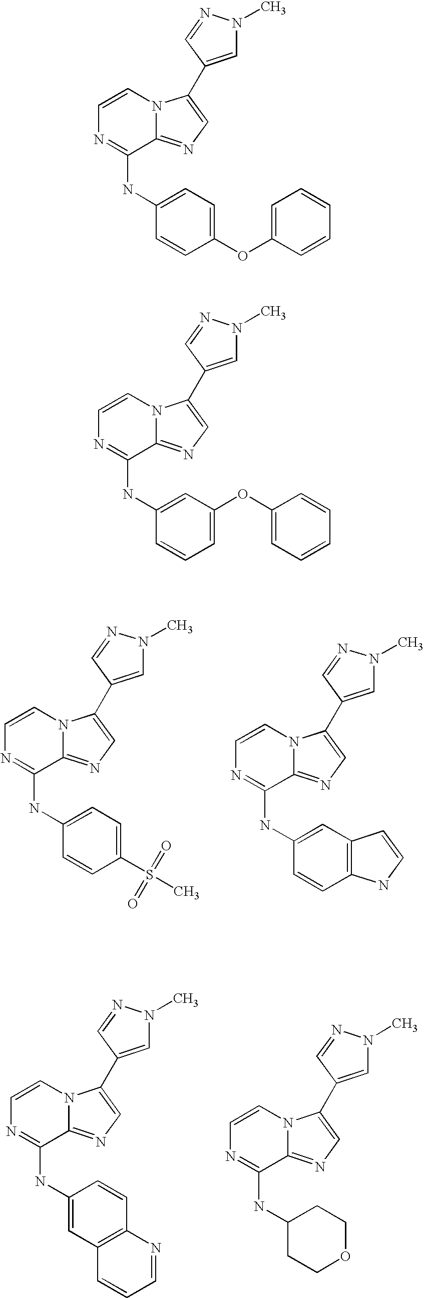 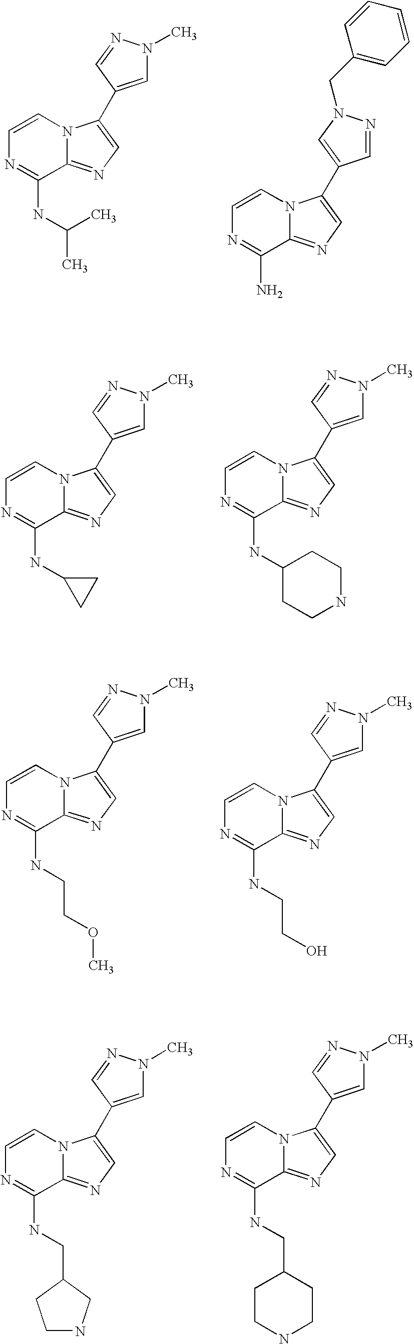 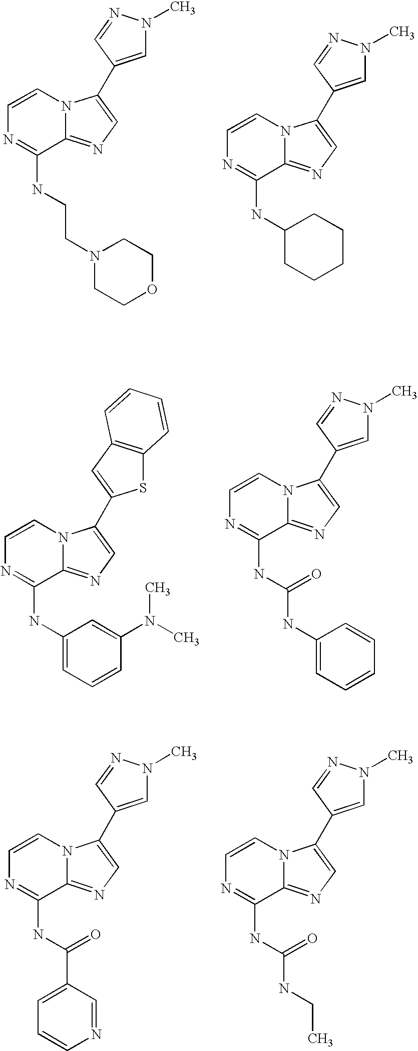 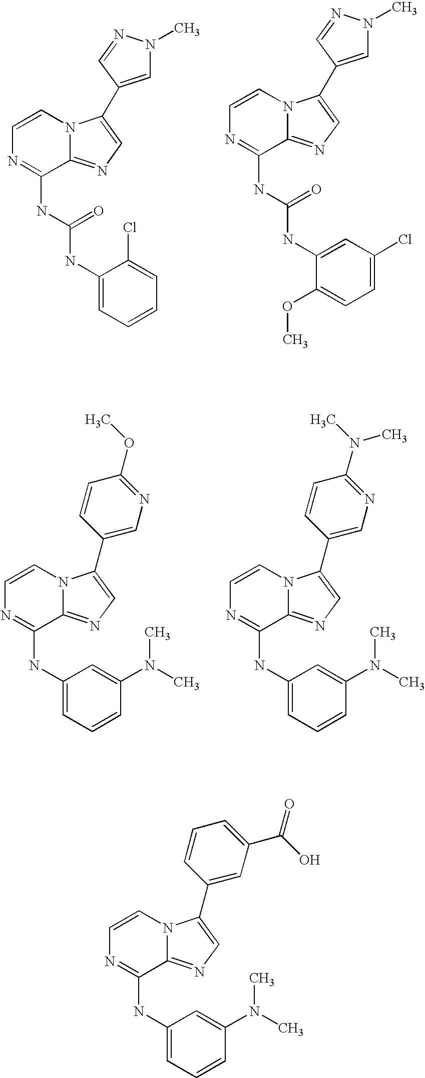 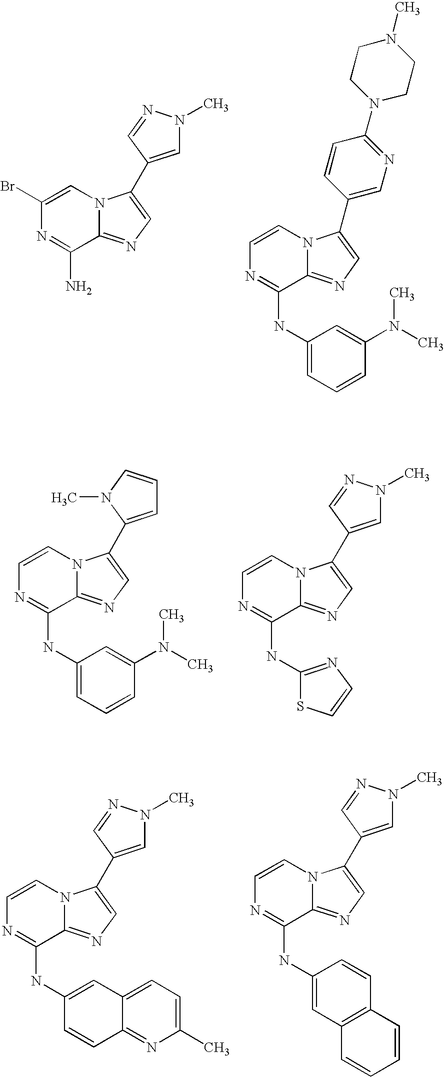 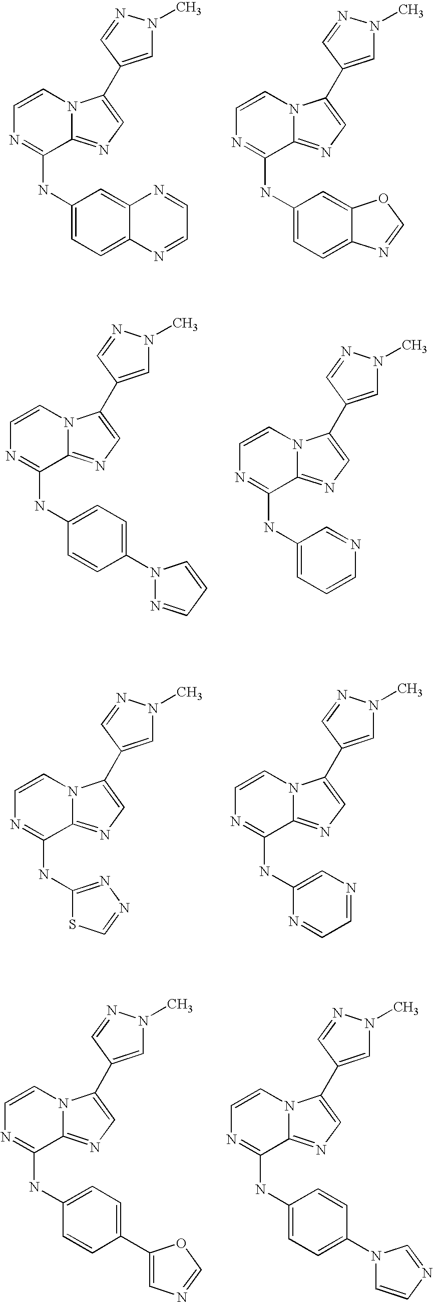 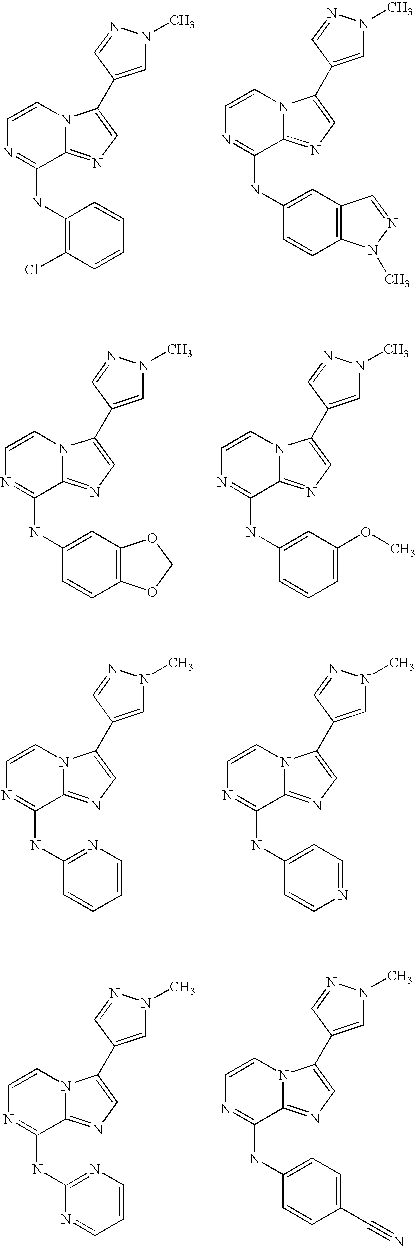 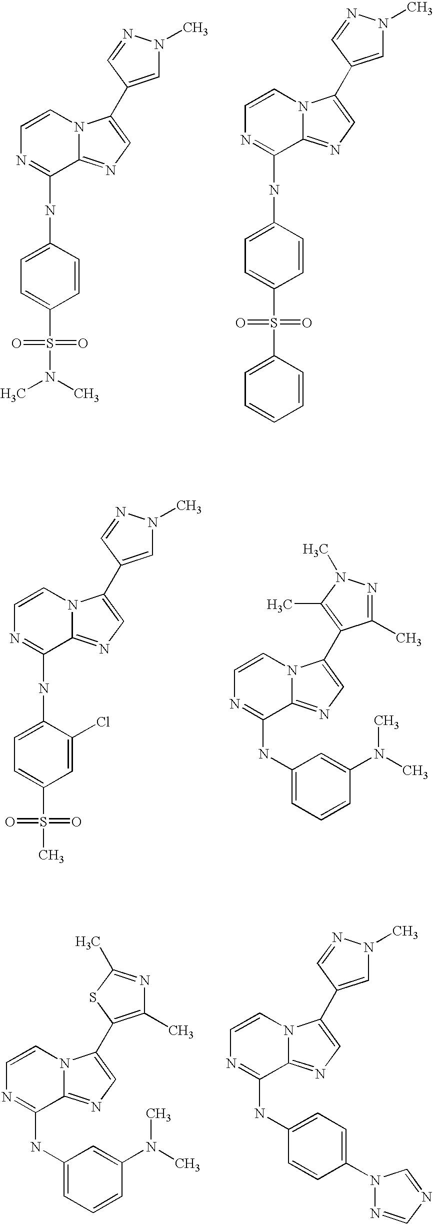 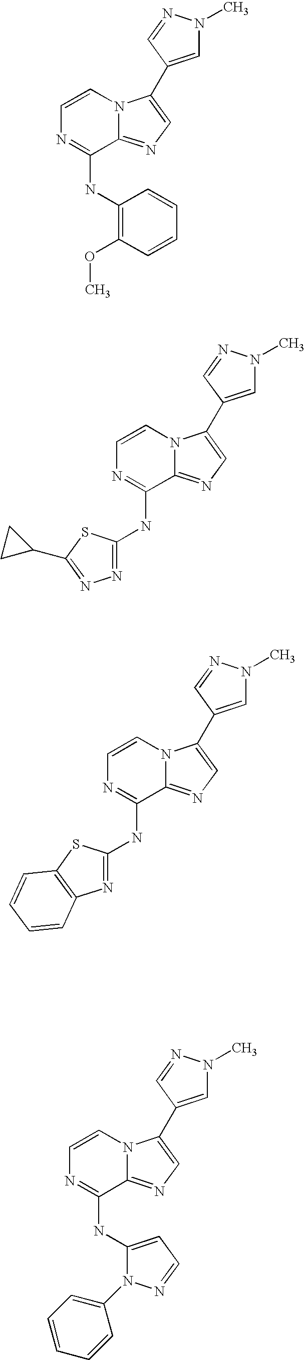 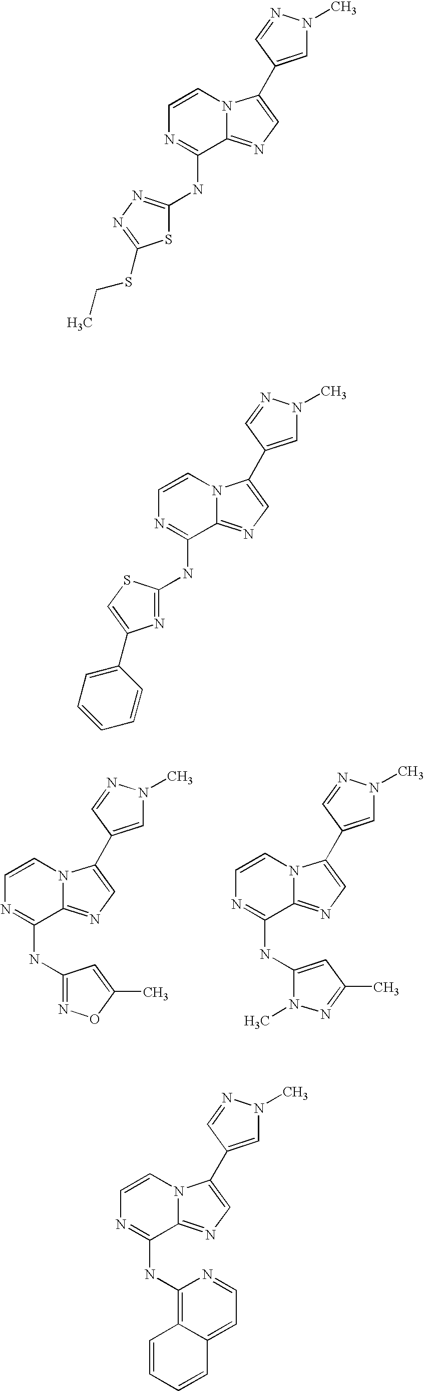 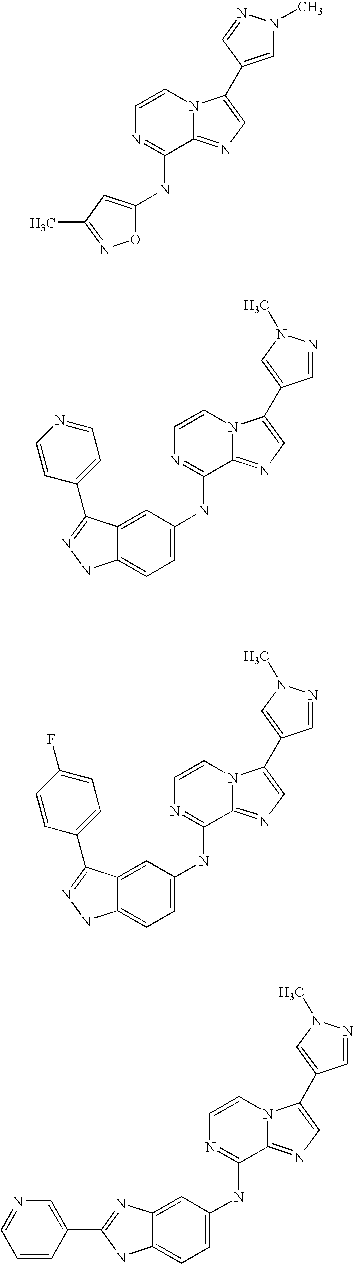 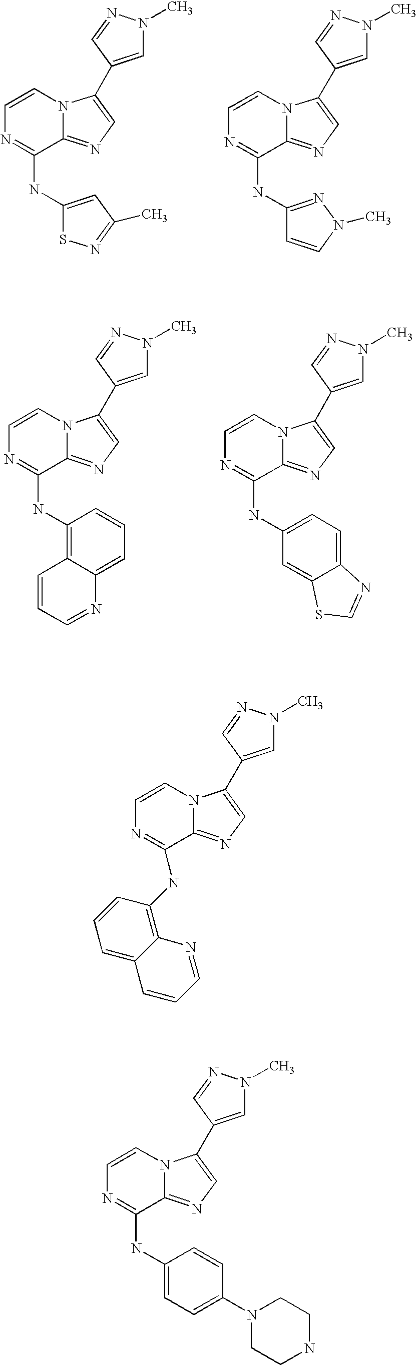 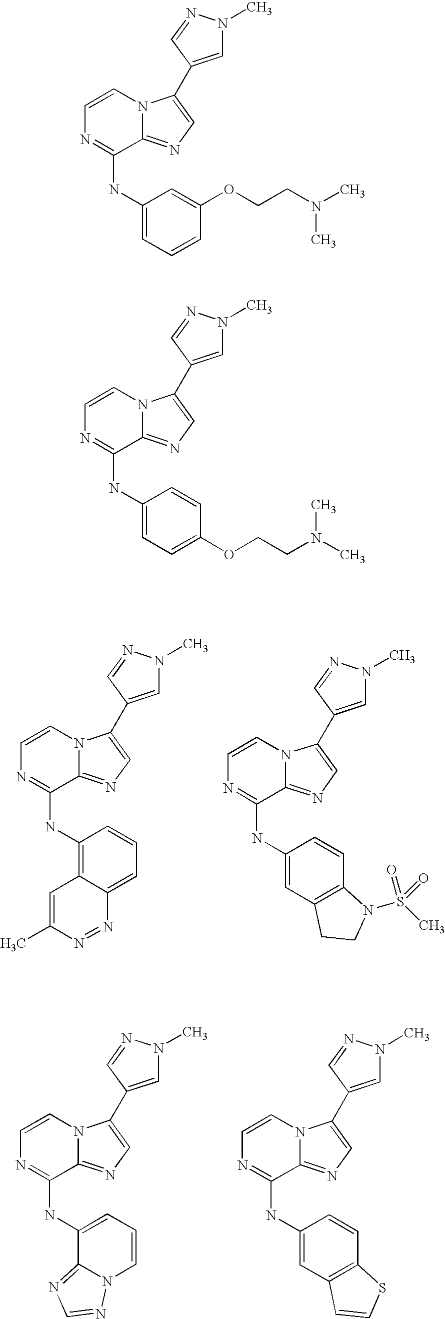 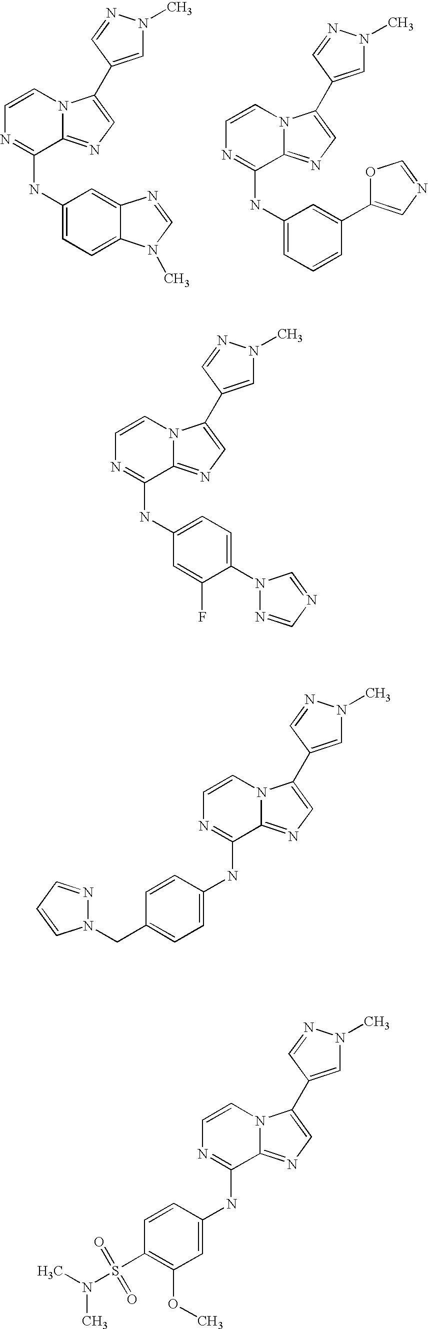 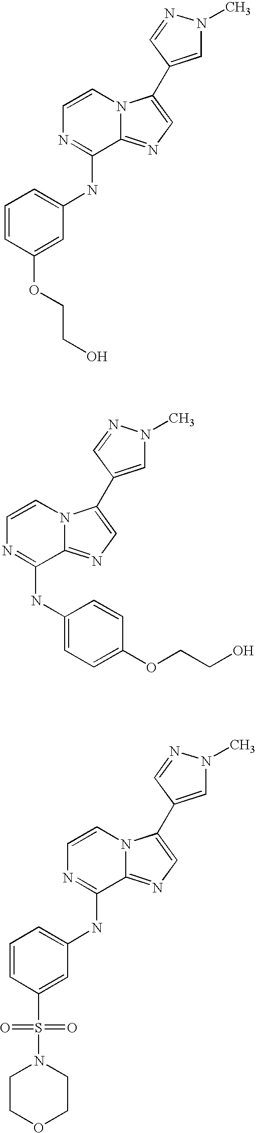 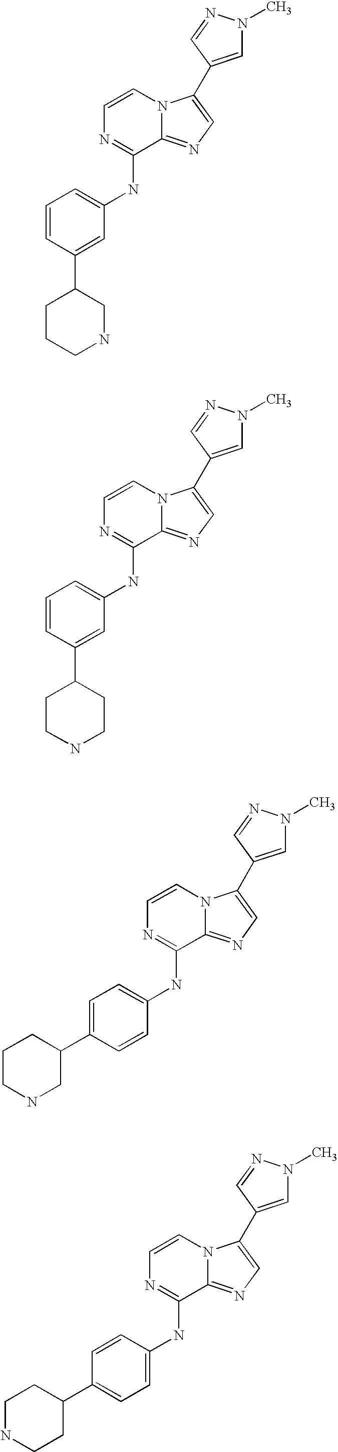 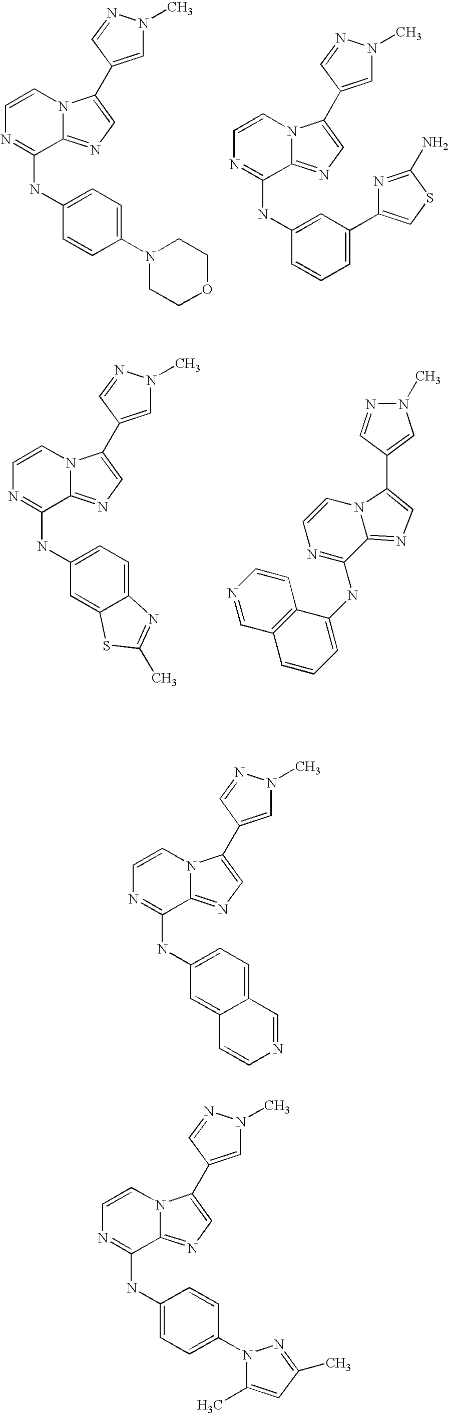 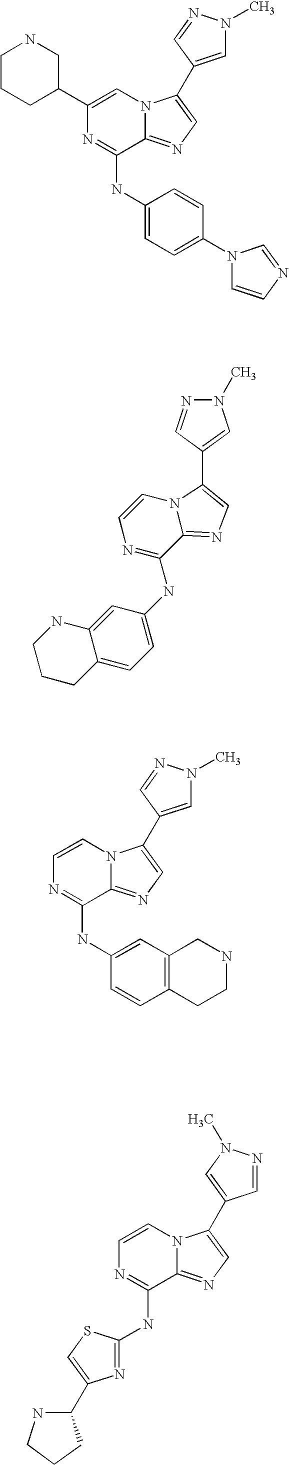 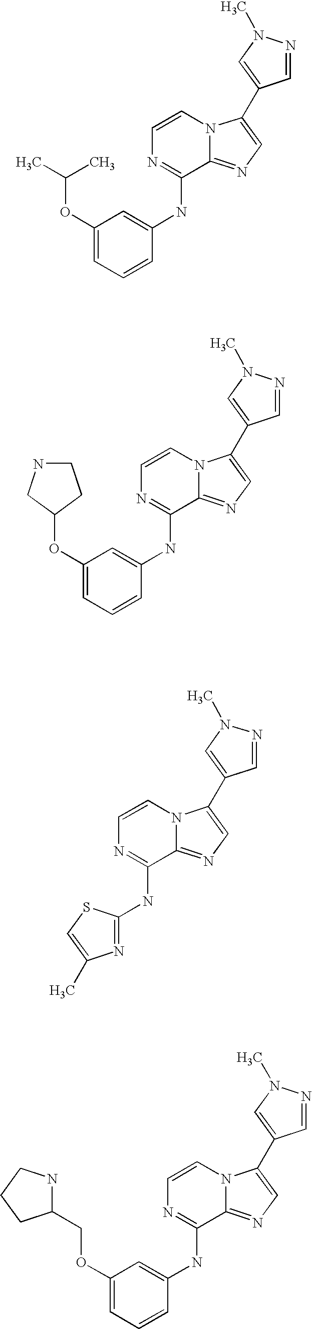 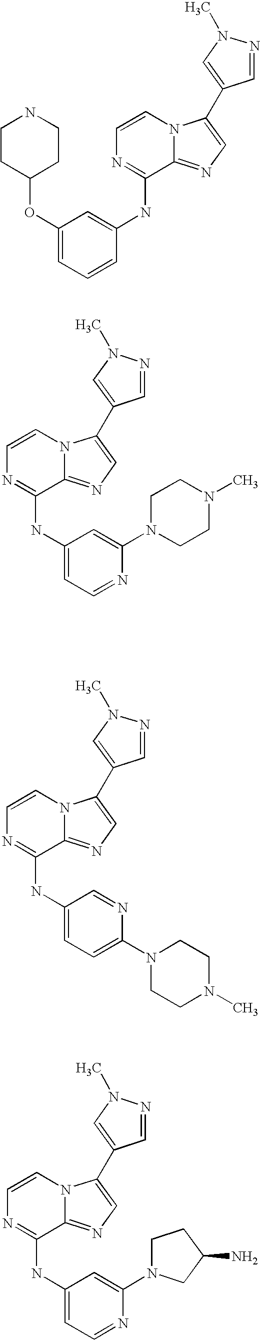 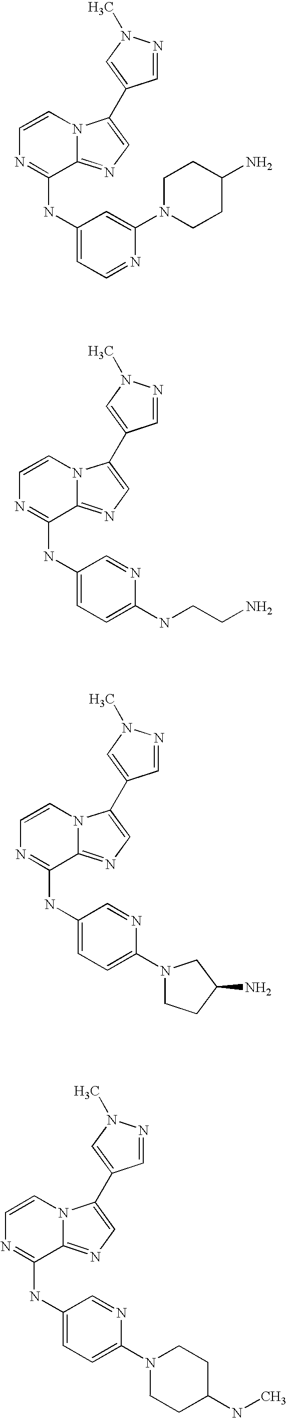 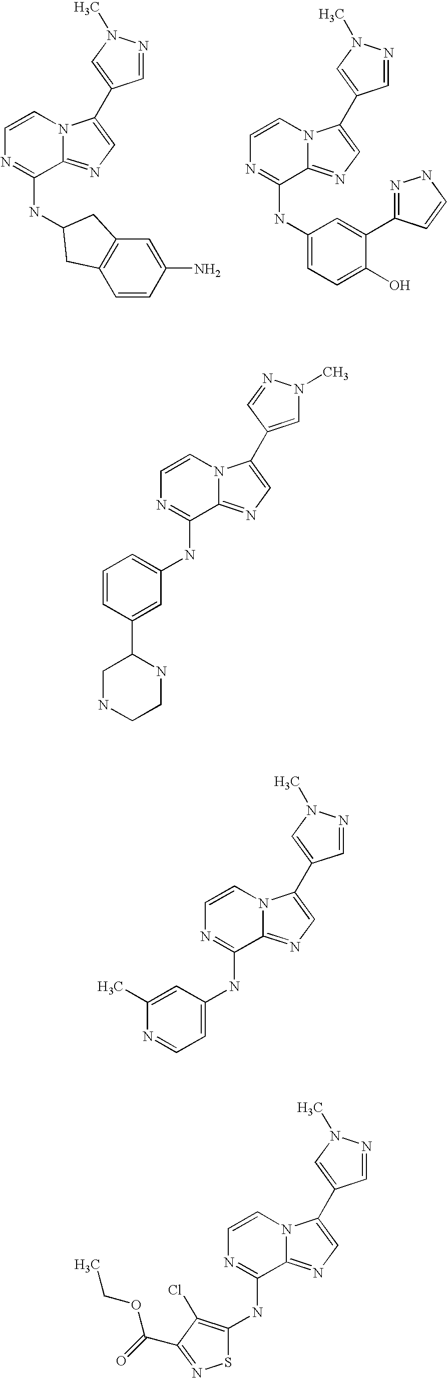 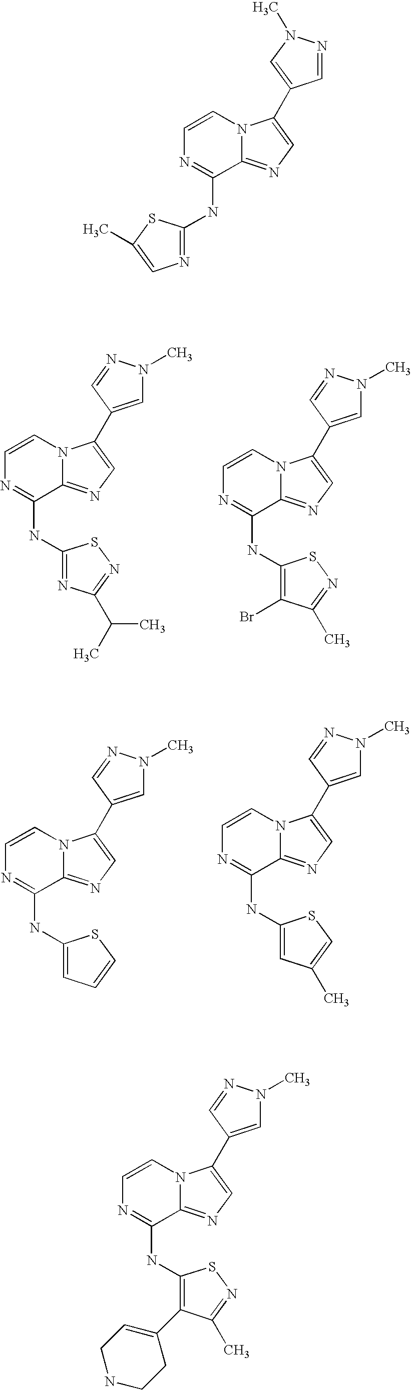 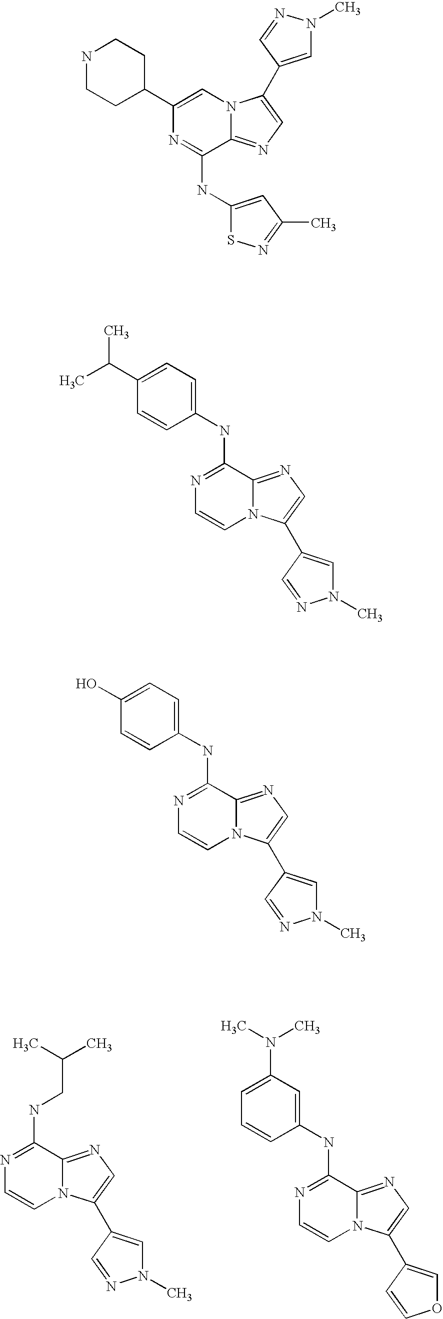 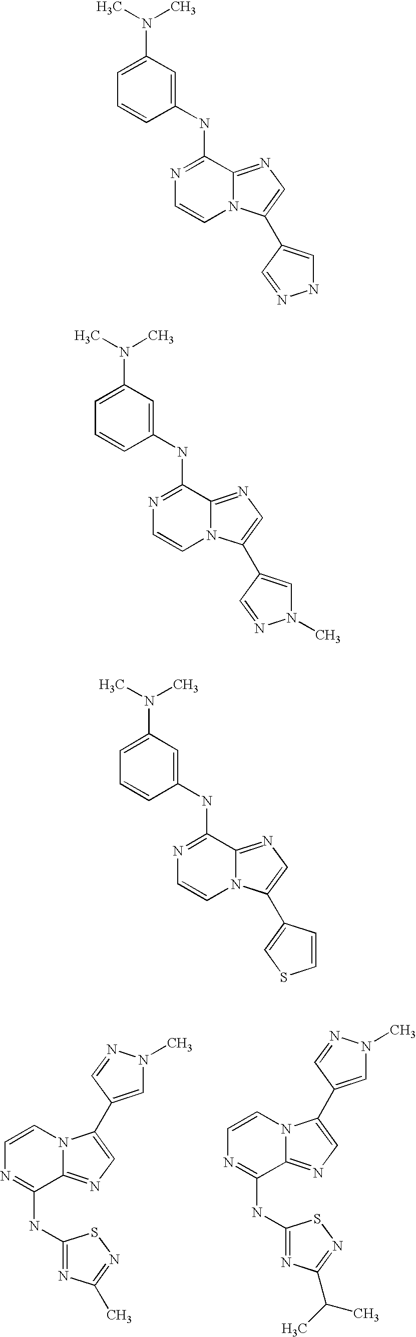 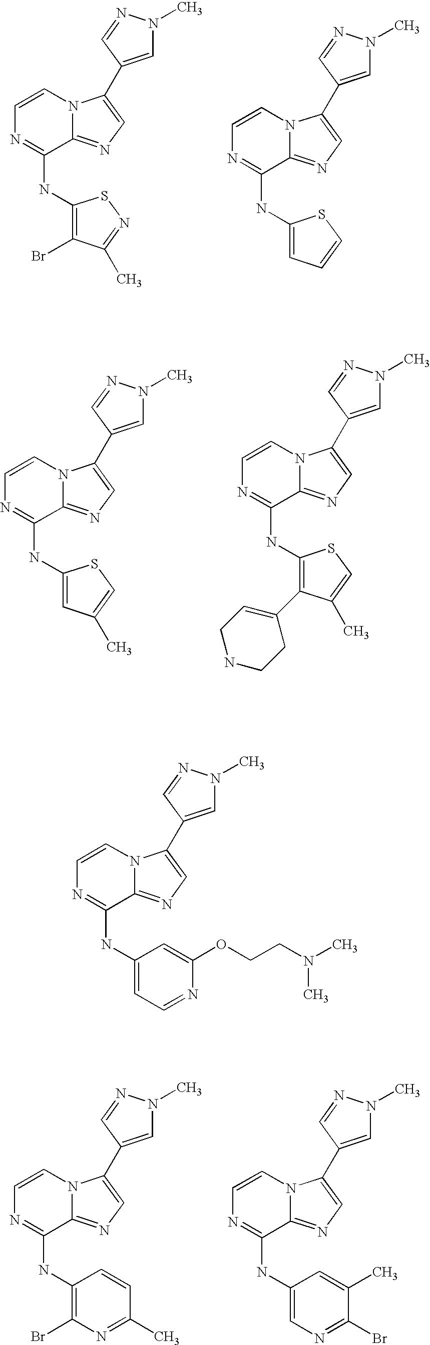 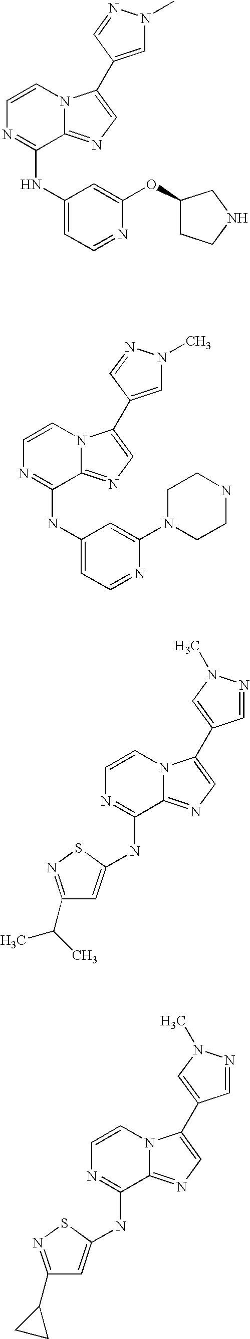 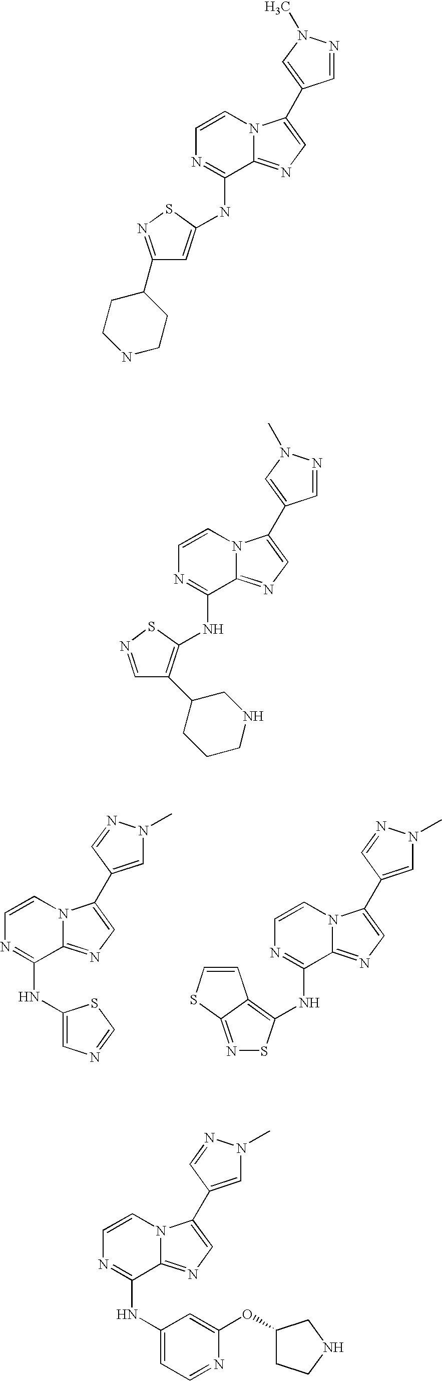 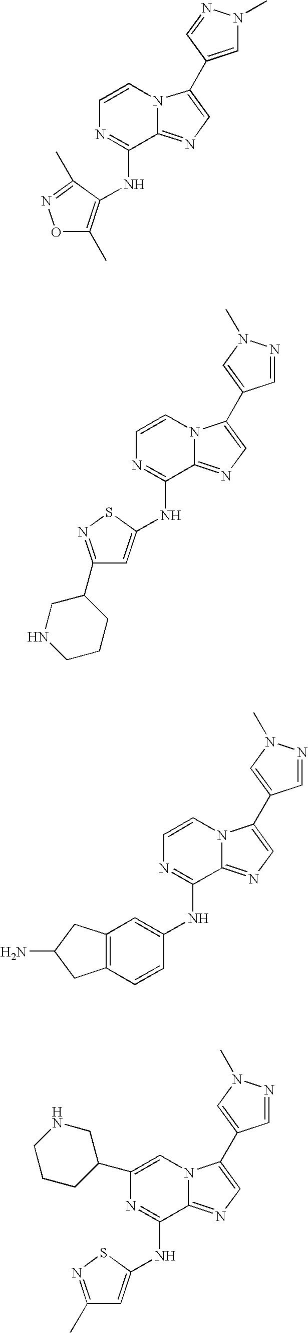 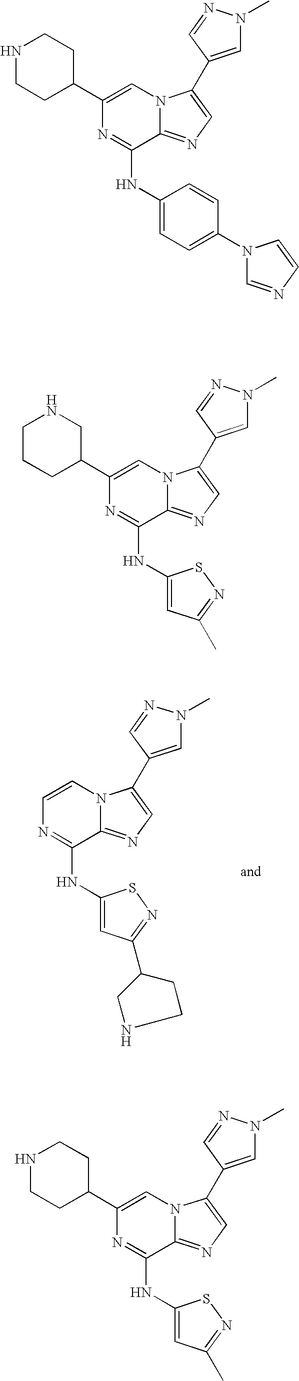
or a pharmaceutically acceptable salt, solvate, ester or prodrug thereof;
h. A compound of the formula: 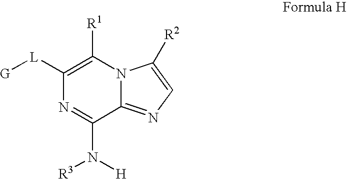
or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷, 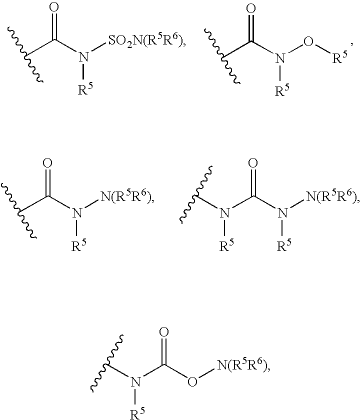
alkyl substituted with 1-6 R⁹ groups which groups can be the same or different with each R⁹ being independently selected, 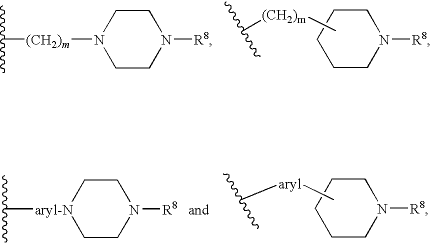
wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,
—N(R⁵)C(O)R⁷ and —N(R⁵)C(O)NR⁵R⁶;
R³ is selected from the group consisting of H, alkyl, aryl, heteroaryl, heterocyclyl, aralkyl, heteroarylalkyl, cycloalkylalkyl, heterocyclylalkyl, 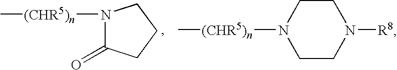
—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₙ—NR⁸, and 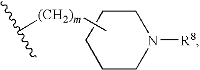
wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —NR⁵R⁶, —C(O₂)R⁵, —C(O)NR⁵R⁶, —SR⁶, —S(O₂)R⁶, —S(O₂)NR⁵R⁶, —N(R)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4; and
p is 0-3;
i. A compound of Formula I: 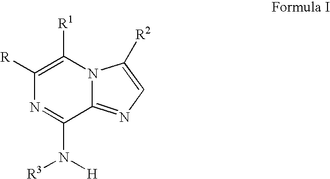
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
—N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
—NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
—CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
X is selected from the group consisting of —NR⁵R⁶, —OR⁵, —SO—R⁵, —SR⁵, SO₂R⁵, heteroaryl, heterocyclyl and aryl, wherein said heteroaryl or aryl can be unsubstituted or substituted with one or more moieties, independently selected from the group consisting of —O-alkyl, alkyl, halo, or NR⁵R⁶;
R⁷ and R⁸ are each independently hydrogen, alkyl, heterocyclyl, aryl, heteroaryl or cycloalkyl;
R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cyclenyl, heterocyclylalkoxyl, —S-alkylheterocyclyl, heterocyclyl, heterocyclenyl, alkylN(alkyl)₂, alkylNH(alkyl), alkylN(alkenyl)₂, -alkylN(alkoxyl)₂, -alkyl-SH, hydroxyalkyl, trihaloalkyl, dihaloalkyl, monohaloalkyl, wherein each of said alkyl, alkenyl, alkoxyalkyl, -alkyl-S-alkyl, aminoalkyl, aryl, heteroaryl, heterocyclenyl, heterocycloalkyl, cycloalkyl, cyclenyl, heterocyclylalkoxyl, —S-alkylheterocyclyl, heterocyclyl, heterocyclenyl, alkyl N(alkyl)₂, alkylNH(alkyl), alkylN(alkenyl)₂, -alkylN(alkoxyl)₂, -alkyl-SH, hydroxyalkyl, trihaloalkyl, dihaloalkyl, monohaloalkyl can be unsubstituted or substituted with one or more moieties independently selected from the group consisting of alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
R⁶ is selected from the group consisting of hydrogen, alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, aminoalkyl, -alkyl-OC(O)alkyl, -alkylOC(O)cycloalkyl, -alkylOC(O)aryl, -alkylOC(O)aralkyl, -alkylOC(O)NR⁵aryl, -alkylOC(O)NR⁵alkyl, -alkylOC(O)NR⁵heterocyclyl, -alkylOC(O)NR⁵heteroaryl -alkylOC(O)NR⁵cycloalkyl, -alkylOC(O)heterocyclyl, alkylC(O)OH, alkylC(O)Oalkyl, -alkylC(O)Ocycloalkyl, -alkylC(O)Oaryl, -alkylC(O)Oaralkyl, -alkylC(O)ONR⁵aryl, -alkylC(O)ONR⁵alkyl, -alkylC(O)ONR⁵heterocyclyl, -alkylC(O)ONR⁵heteroaryl -alkylC(O)ONR⁵cycloalkyl, -alkylC(O)Oheterocyclyl, alkylC(O)OH, and alkylC(O)Oalkyl wherein each of said aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, -alkyl-OC(O)alkyl, -alkylOC(O)cycloalkyl, -alkylOC(O)aryl, -alkylOC(O)aralkyl, -alkylOC(O)NR⁵aryl, -alkylOC(O)NR⁵alkyl, -alkylOC(O)NR⁵heterocyclyl, -alkylOC(O)NR⁵heteroaryl -alkylOC(O)NR⁵cycloalkyl, -alkylOC(O)heterocyclyl, alkylC(O)OH, alkylC(O)Oalkyl, -alkylC(O)Ocycloalkyl, -alkylC(O)Oaryl, -alkylC(O)Oaralkyl, -alkylC(O)ONR⁵aryl, -alkylC(O)ONR⁵alkyl, -alkylC(O)ONR⁵heterocyclyl, -alkylC(O)ONR⁵heteroaryl -alkylC(O)ONR⁵cycloalkyl, -alkylC(O)Oheterocyclyl, alkylC(O)OH, and alkylC(O)Oalkyl, can be unsubstituted or substituted with one or more moieties independently selected from the group consisting of alkyl, alkenyl, aryl, cyclenyl, cycloalkyl, arylalkyl, cyclenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, aminoalkyl, amino, aminodialkyl, aminocycloalkyl, halo, trihaloalkyl, dihaloalkyl, and monohaloalkyl;
further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cyclenylalkyl, cycloalkylalkyl, heteroarylalkyl, heterocyclenylalkyl, heterocycloalkylalkyl, heteroaryl, aryl, cyclenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, heterocyclenyl, halo, trihaloalkyl, dihaloalkyl, CN and monohaloalkyl.
j. A compound having the formula: 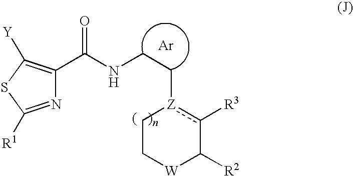
or a pharmaceutically acceptable salt, solvate, ester, prodrug or stereoisomer thereof, wherein the dashed lines indicate an optional and additional bond, and wherein:
R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;
R² is —H, -alkyl, —NH₂ or —CH₂NH₂;
R³ is —H, -alkyl, —NH₂ or —CH₂NH₂;
each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;
R⁵ is —H, -alkyl, -aryl, -heteroaryl, —NHOH;
R⁶ is —H, -alkyl or —CF₃;
R⁷ is —H, —OH, —C₁-C₆ alkyl, —O—(C₁-C₆ alkyl), or —CF₃;
R⁸ is —H, alkyl, aryl, heterocyclyl, heteroaryl or cycloalkyl;
Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen
W is —NH— or C(R⁴)₂—, wherein both R⁴ groups and the carbon atom to which they are attached can combine to form a five to seven membered heterocyclyl or heteroaryl group;
Y is —H, -halo, -alkyl or —CN;
Z is —CR⁷— or —N—, when the optional additional bond is absent, and —C— when the optional additional bond is present;
n is an integer ranging from 0 to 2; and
(k) A compound of Formula K: 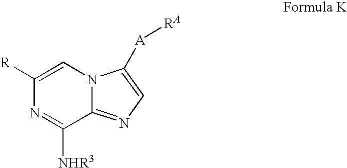
or a pharmaceutically acceptable salt, solvate, ester, prodrug or stereoisomer thereof, wherein:
R is H, halo or alkyl;
R³ is heteroaryl-X, wherein X is heterocyclylalkyl- wherein said heterocyclyl can be unsubstituted or optionally substituted with 1-4 alkyl moieties;
A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;
R^{A} is —(CH₂)₁₋₄heteroaryl, 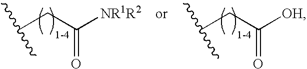
wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
R¹ and R² together with the N to which each is attached, form a heterocyclic group selected from the group consisting of 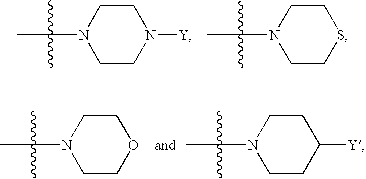
wherein
Y is alkoxyalkyl, hydroxyalkyl, dialkylaminoalkyl or alkyl, further wherein Y′ is hydroxyl.

2. A method of treating or ameliorating cancer comprising administering sequentially to a mammal in need of such treatment an amount of at least one first compound, wherein said first compound is an anti-mitotic agent selected from the group consisting of a taxane, paclitaxel, docetaxel, a Cenp-E Inhibitor, Abraxane, Epothilone, Monastrol, a KSP inhibitor, Ispinesib, and a compound of Formulas A-D shown in paragraphs a-d below:
a. A compound represented by the structural Formula A: 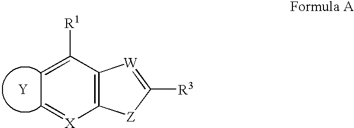
or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷. —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —(C₁-C₆ alkyl)-OR¹⁰, —CN, —NR¹⁰R¹¹, —C(O)OR¹⁰, —C(O)NR¹⁰R¹¹, —CF³, OCF₂, —NR¹⁰)C(O)OR¹¹, and —NR¹⁰C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆₋OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₄SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
b. A compound represented by the structural Formula B: 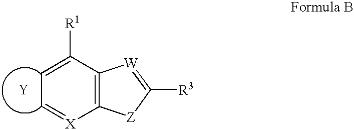
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵, —C(O)N(R⁷)(CR⁴⁰R⁴¹)₁₋₅—OR⁷, —C(S)NR⁷(CH₂)₁₋₅NR⁴R⁵, and —C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl,
—OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰ NR¹⁰R¹¹, and —NR¹⁰)C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,
—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —(C₁-C₆ alkyl)-OR¹⁰,
—CN, —NR¹⁰R¹¹, —C(O)R¹⁰, —C(O)OR¹⁰, —C(O)NR¹⁰R¹¹, —CF₃, —OCF₃,
—N(R¹⁰)C(O)R¹¹, and —NR¹⁰C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;
c. 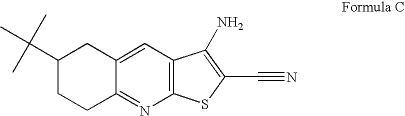
or a pharmaceutically acceptable salt, solvate, or ester thereof; and
d. A compound of Formula D 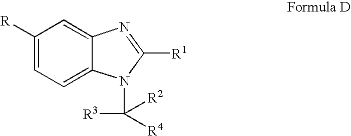
or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
followed by administering one or more of a second compound, wherein said second compound is an aurora kinase inhibitor selected from the group consisting of compounds represented by Formulas E-K shown below in paragraphs e-k:
e. A compound represented by the structural Formula E: 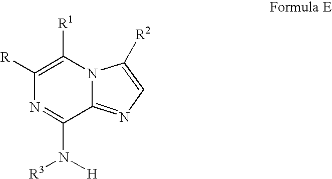
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
—S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring;
f. A compound represented by the structural formula: 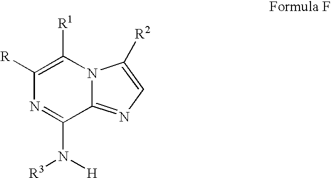
or a pharmaceutically acceptable salt, solvate, ester or prodrug thereof, wherein:
R is selected from the group consisting of H, halogen, aryl, heteroaryl, cycloalkyl, arylalkyl, heterocyclyl, heterocyclylalkyl, alkenyl, alkynyl,
—C(O)R⁷, 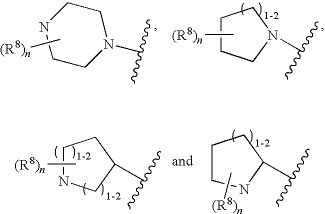
wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃, —OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R¹ is H, halogen or alkyl;
R² is alkyl;
R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₘ—NR⁸, —(CHR⁵)ₙ—CH(aryl)₂, 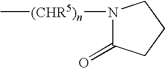
and 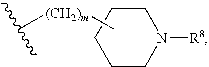
wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H or alkyl;
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶, —C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4;
n is 1-4; and
p is 0-3;
g. A compound selected from the compounds of the formulas: 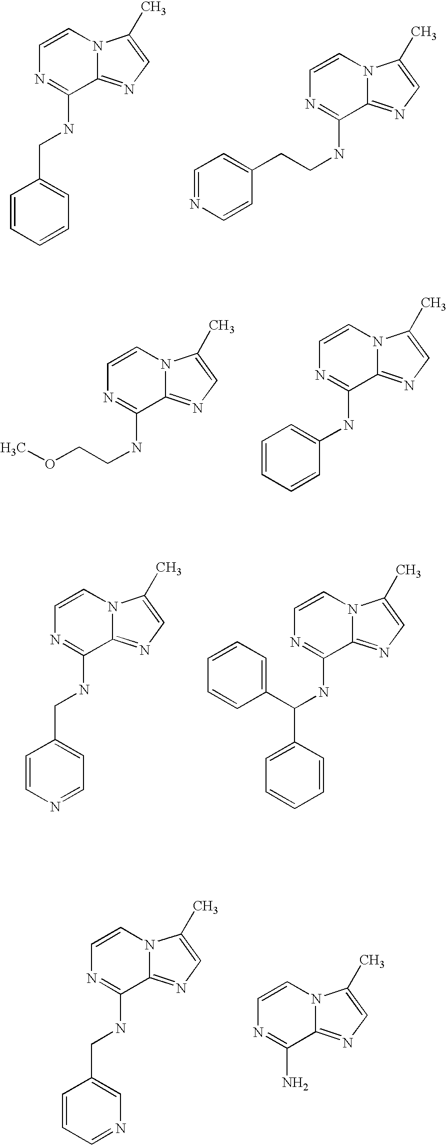 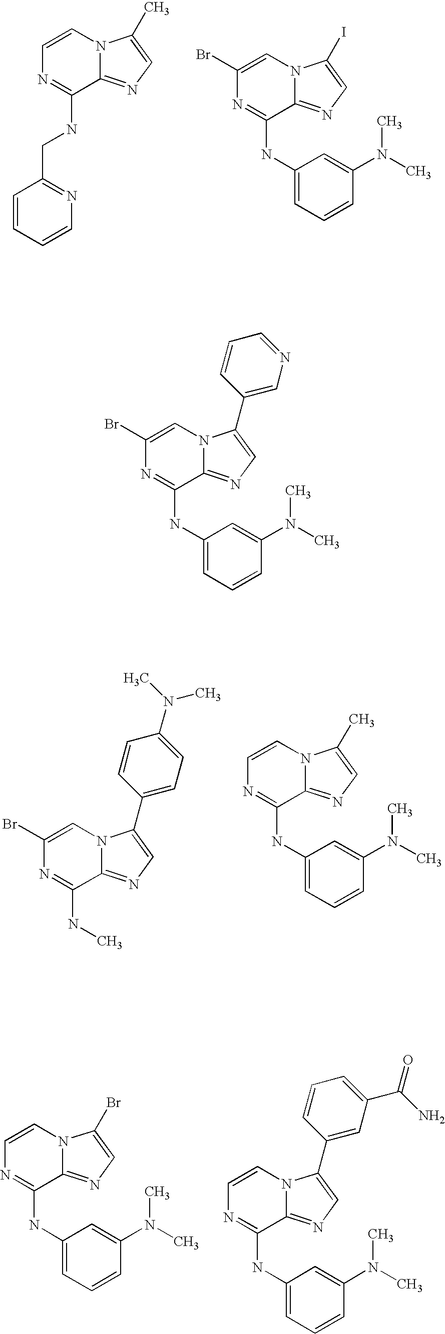 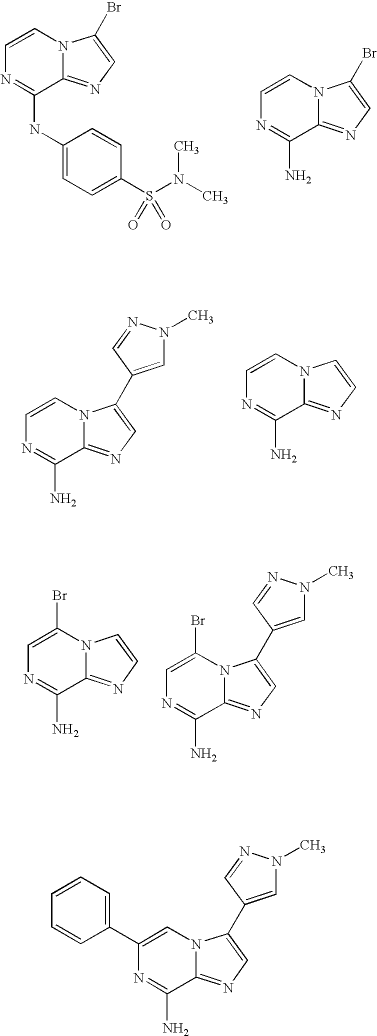 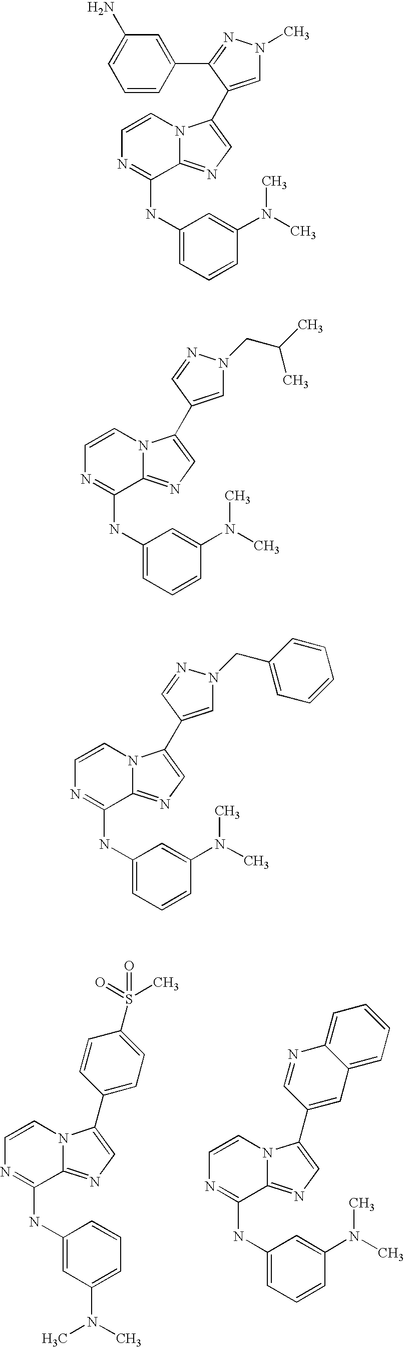 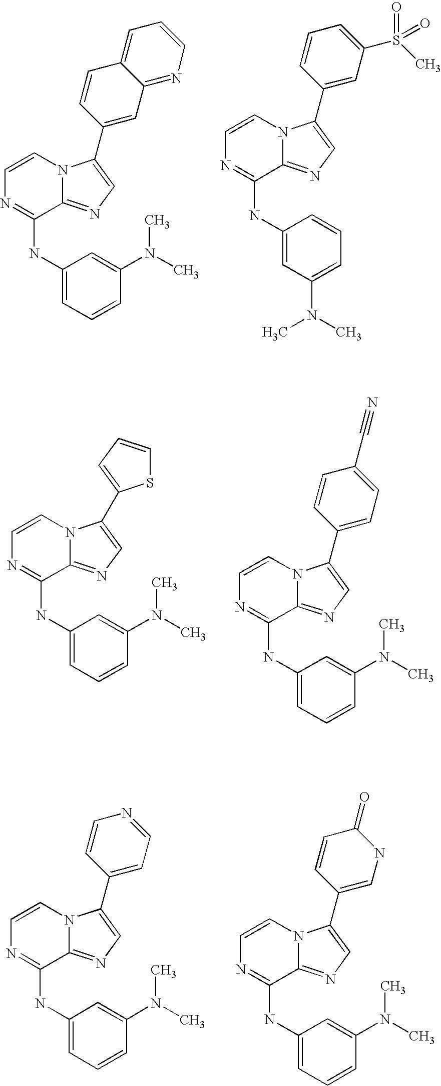 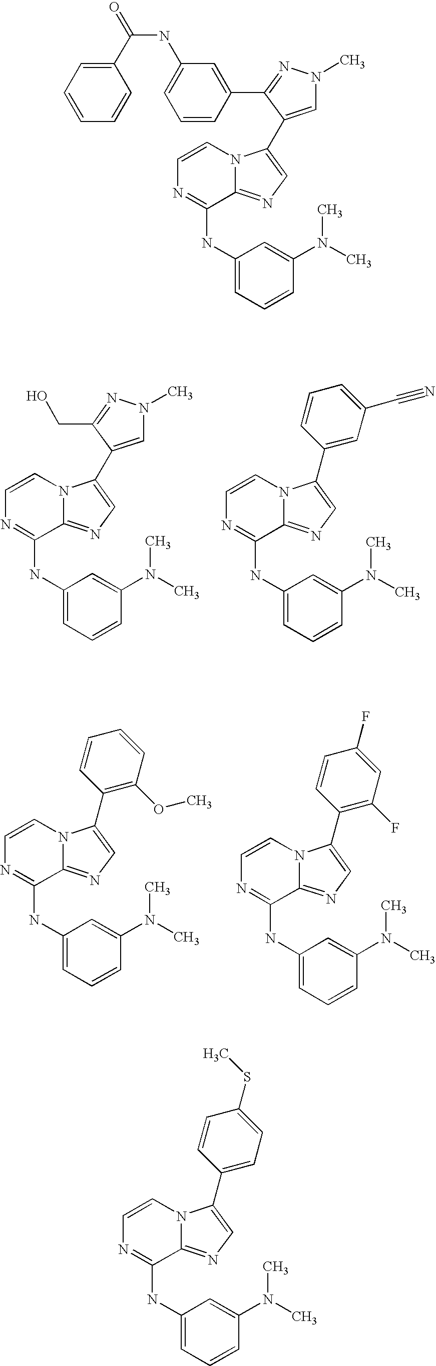 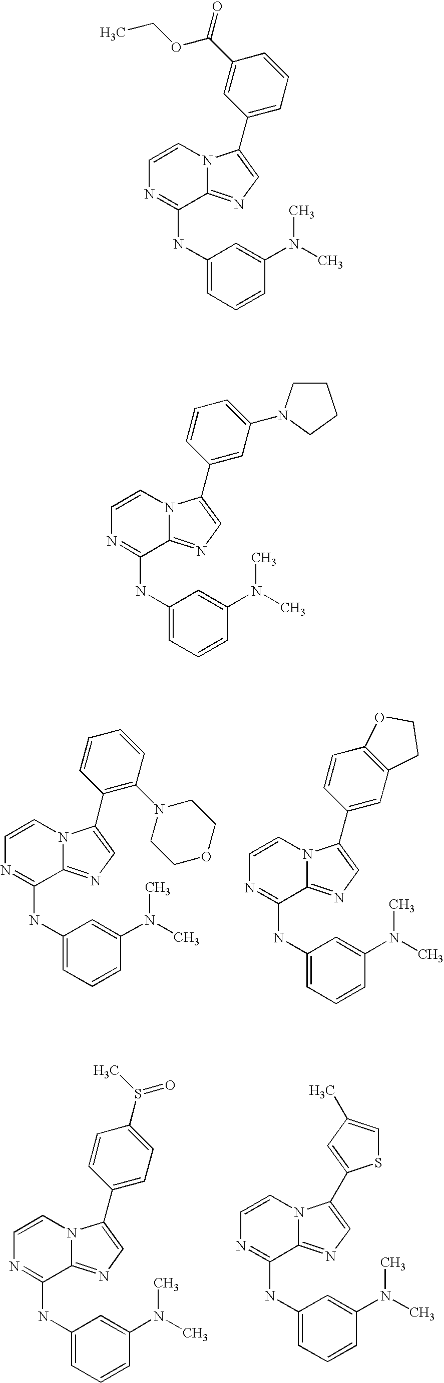 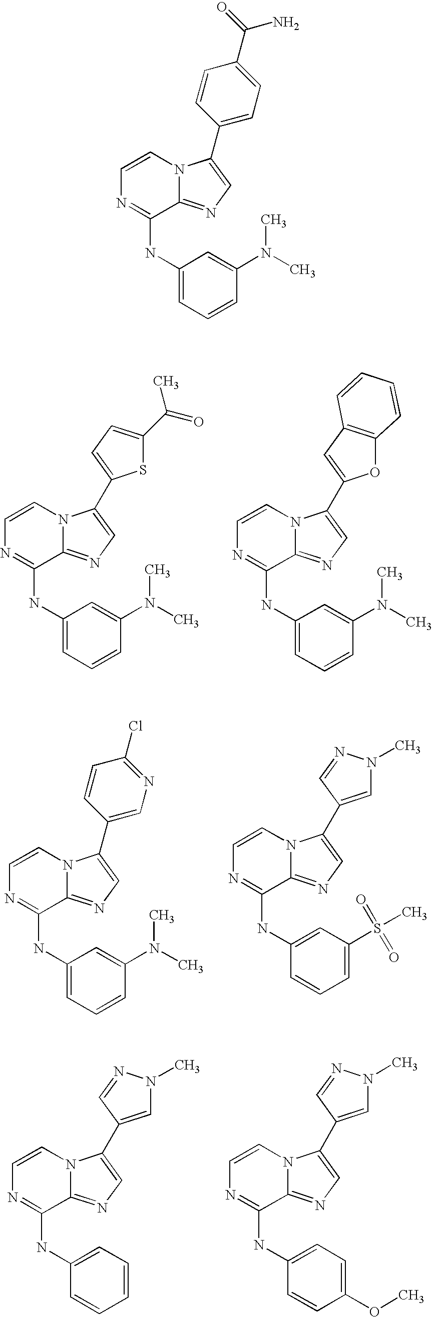 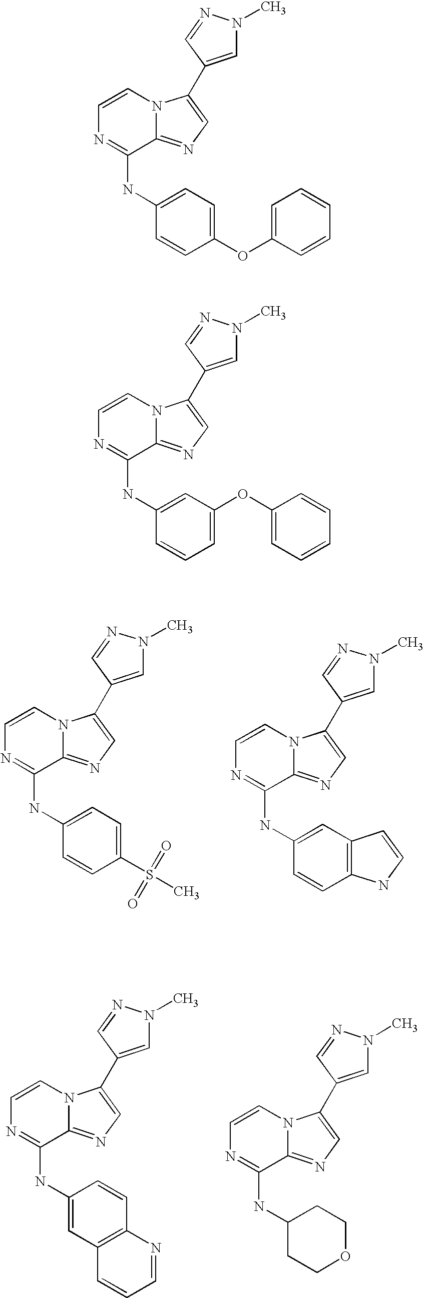 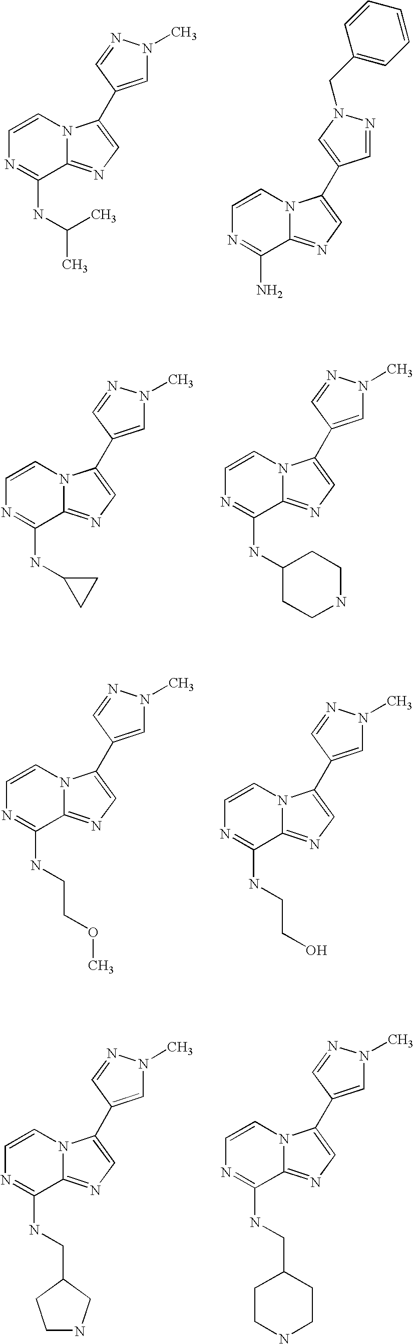 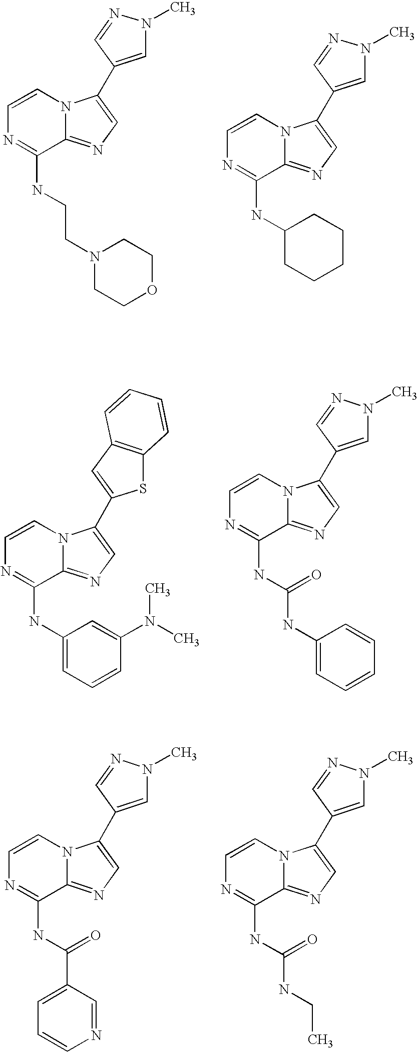 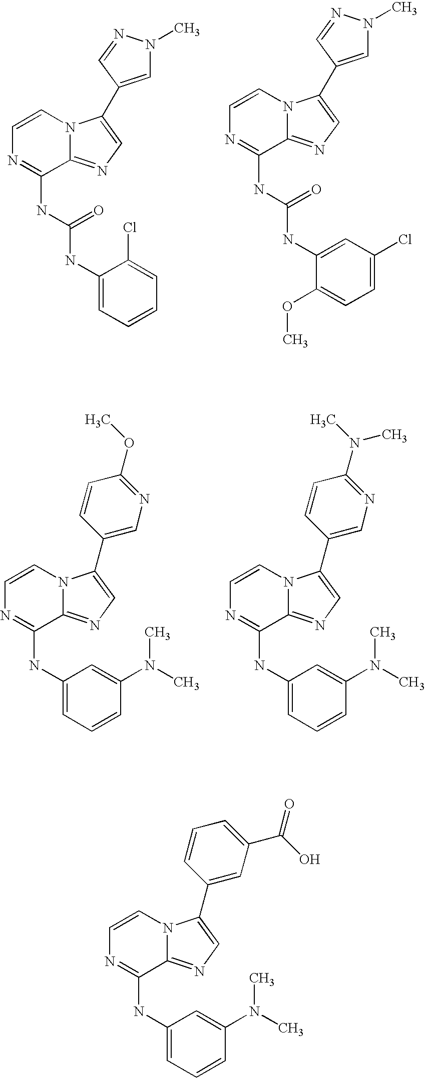 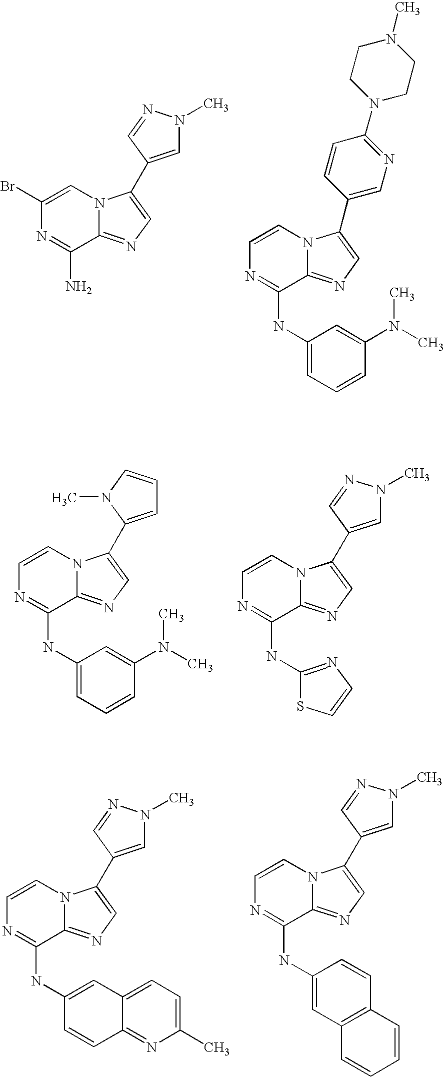 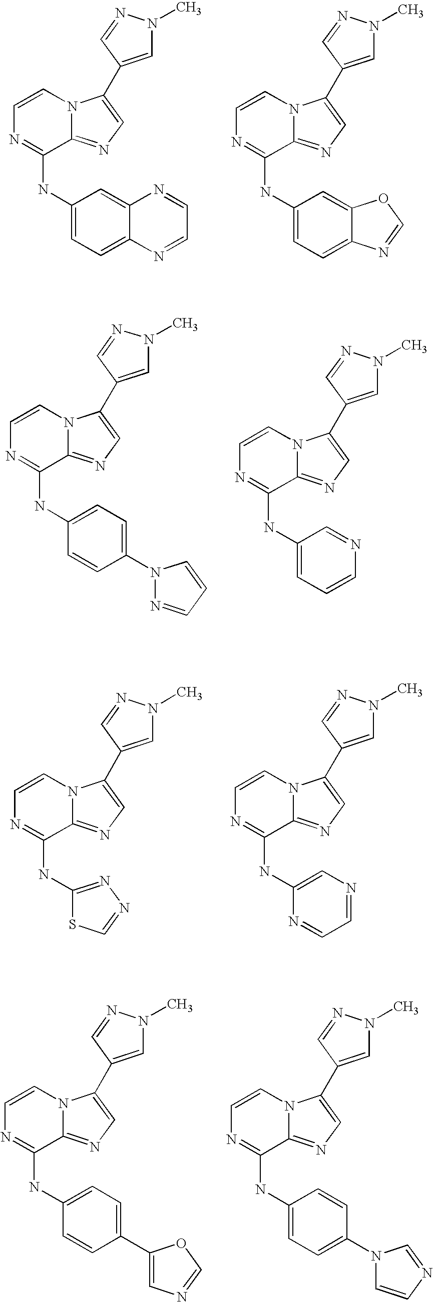 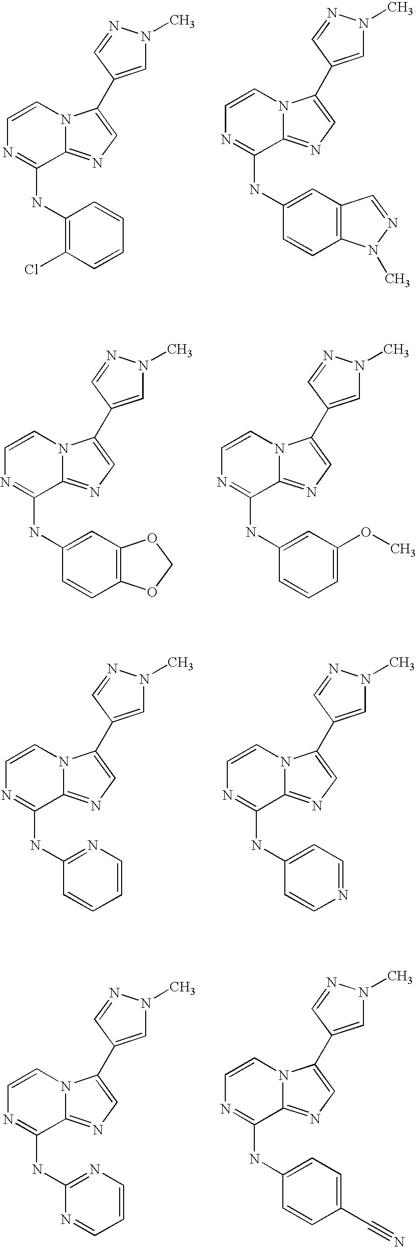 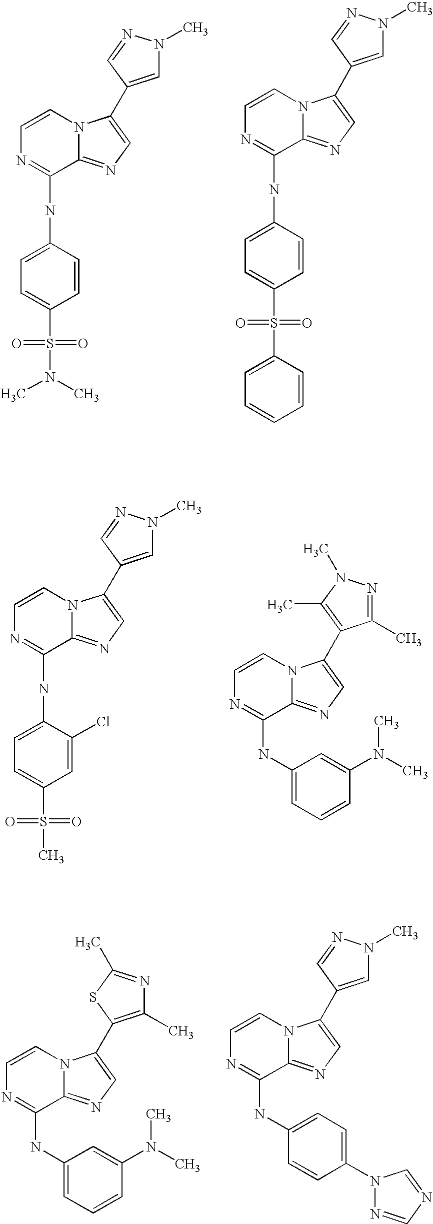 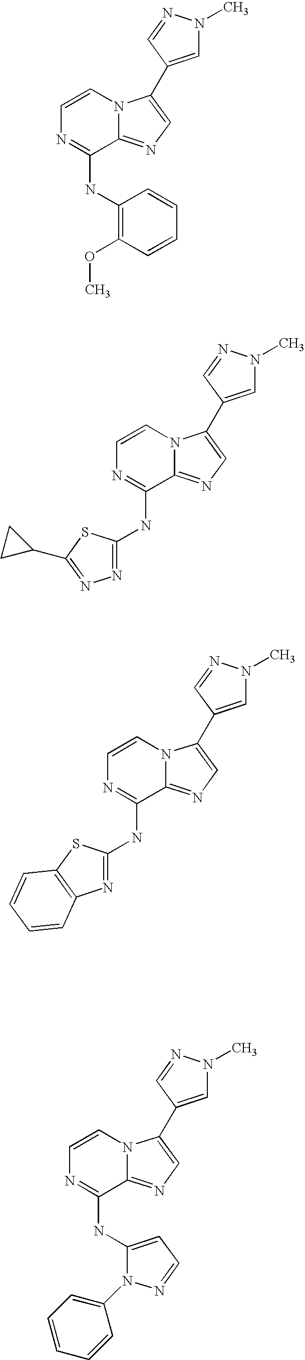 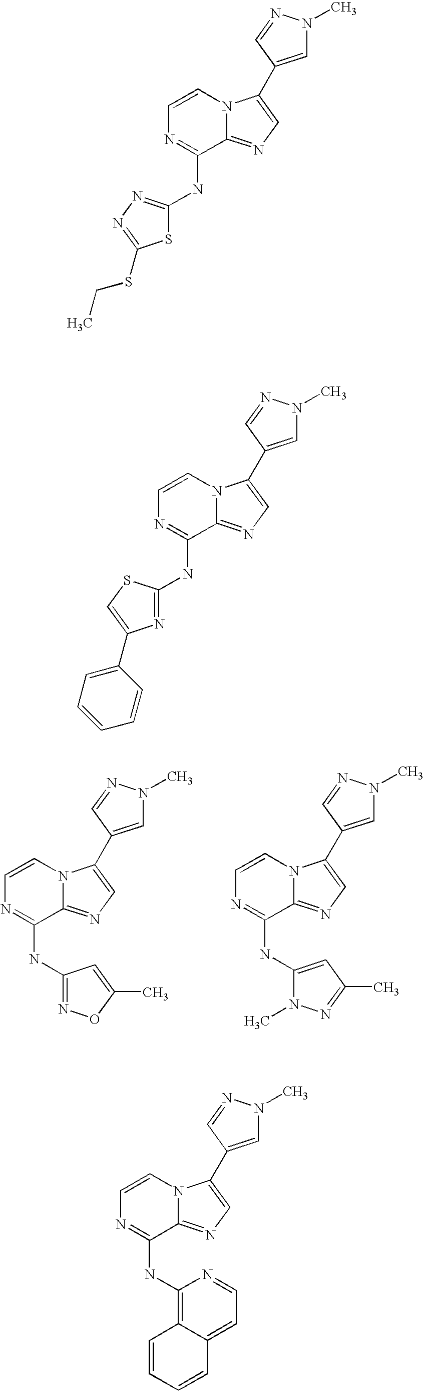 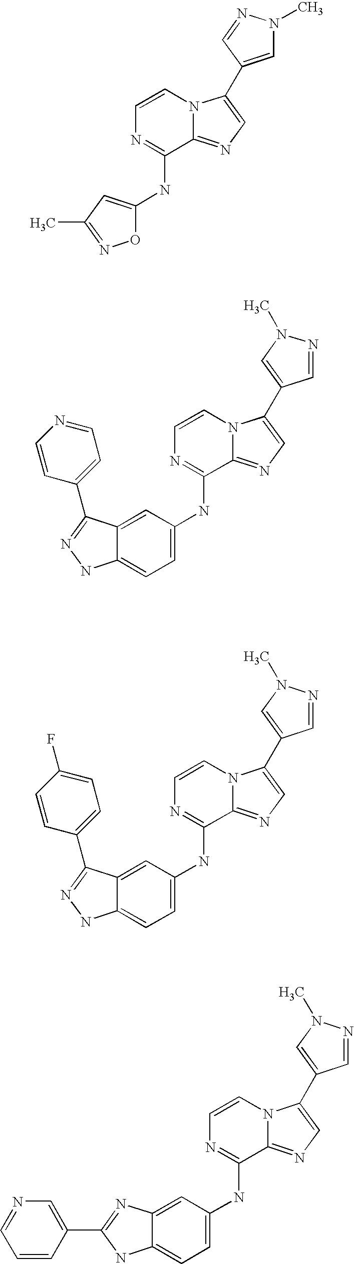 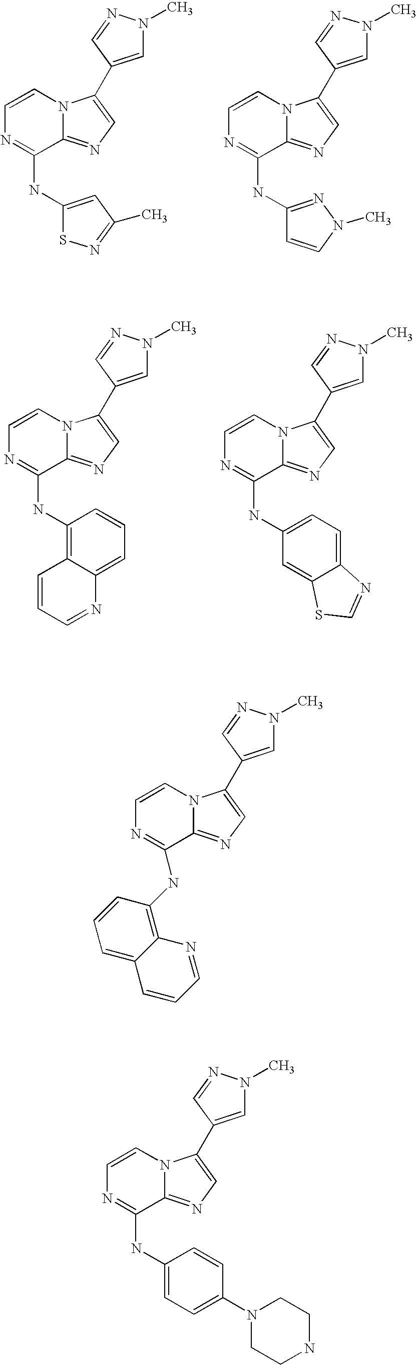 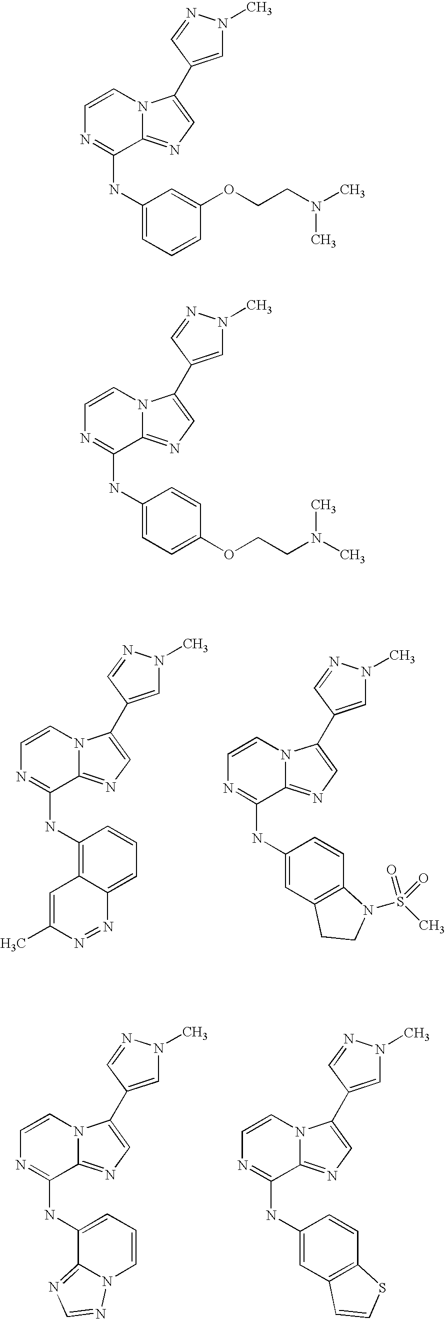 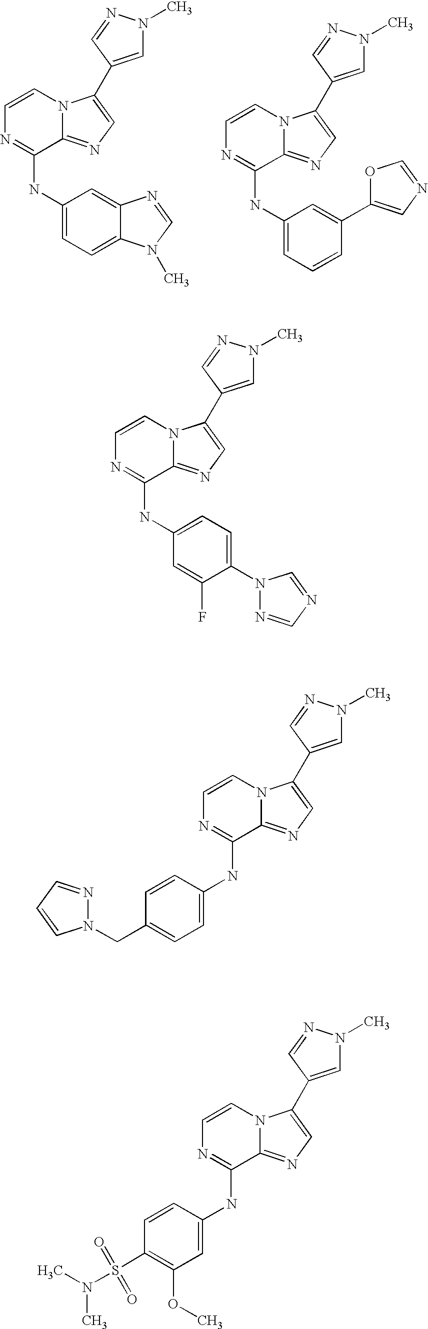 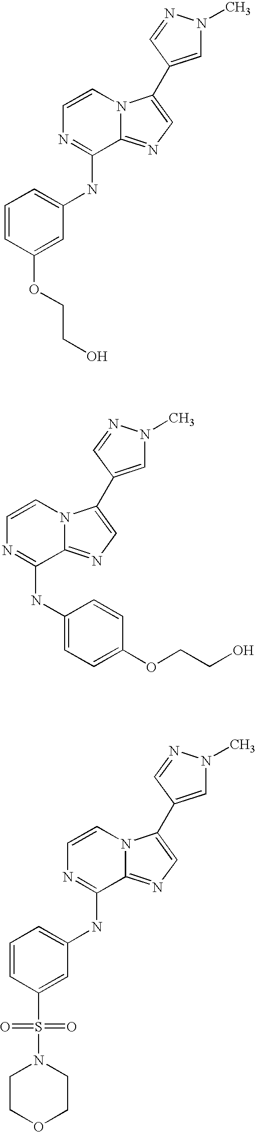 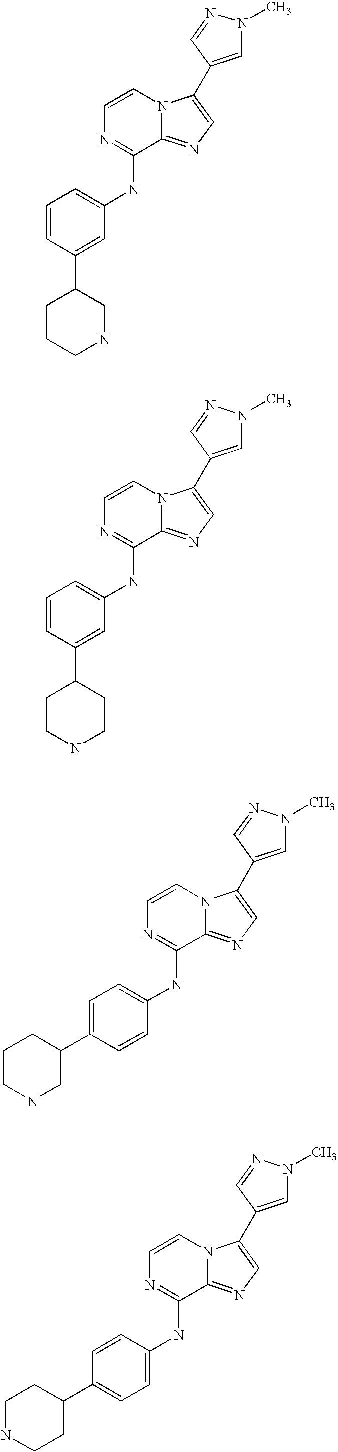 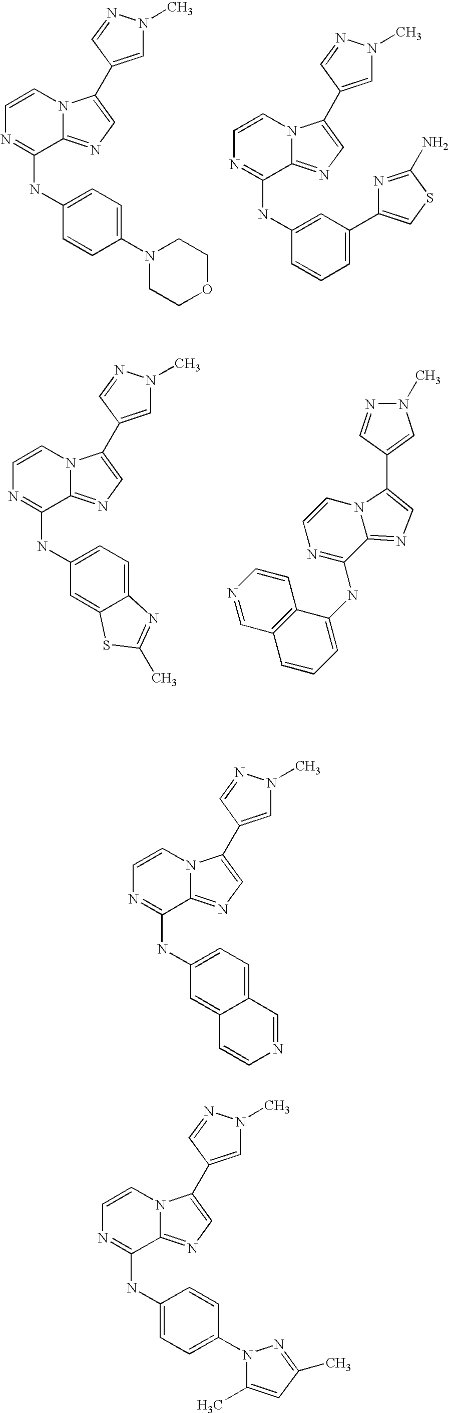 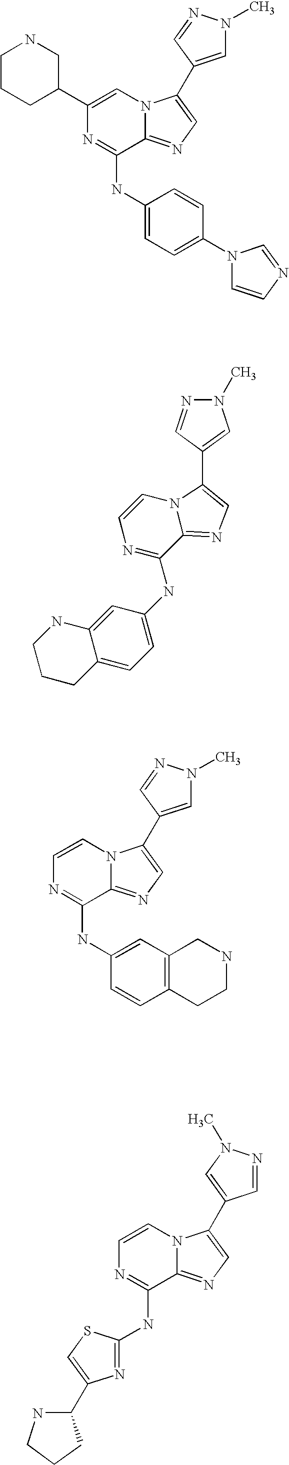 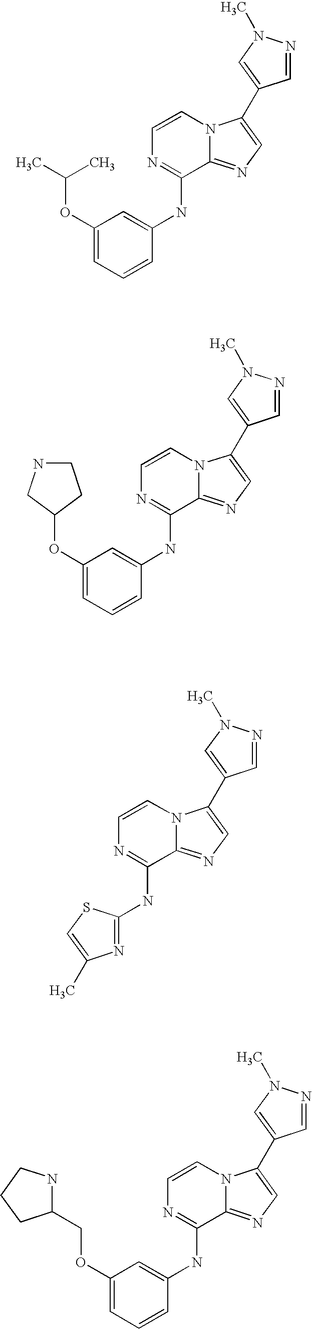 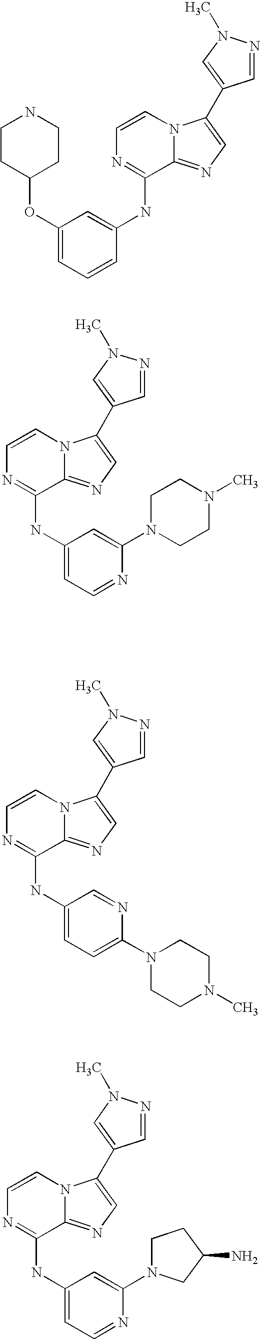 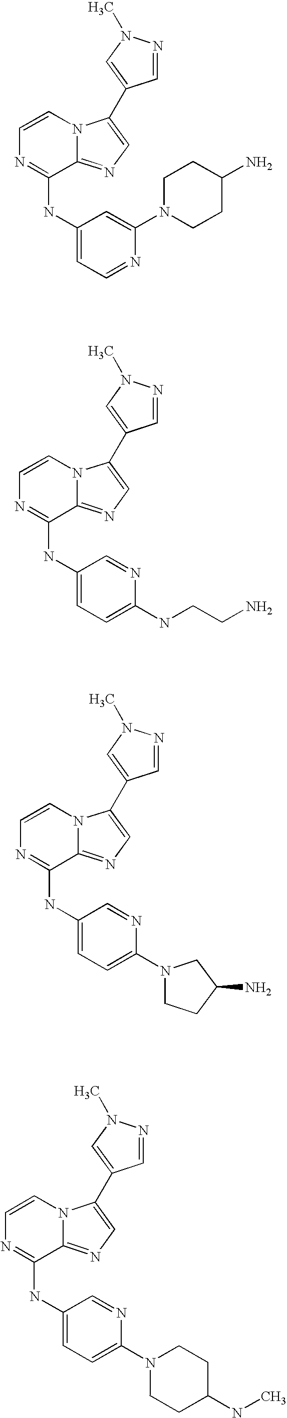 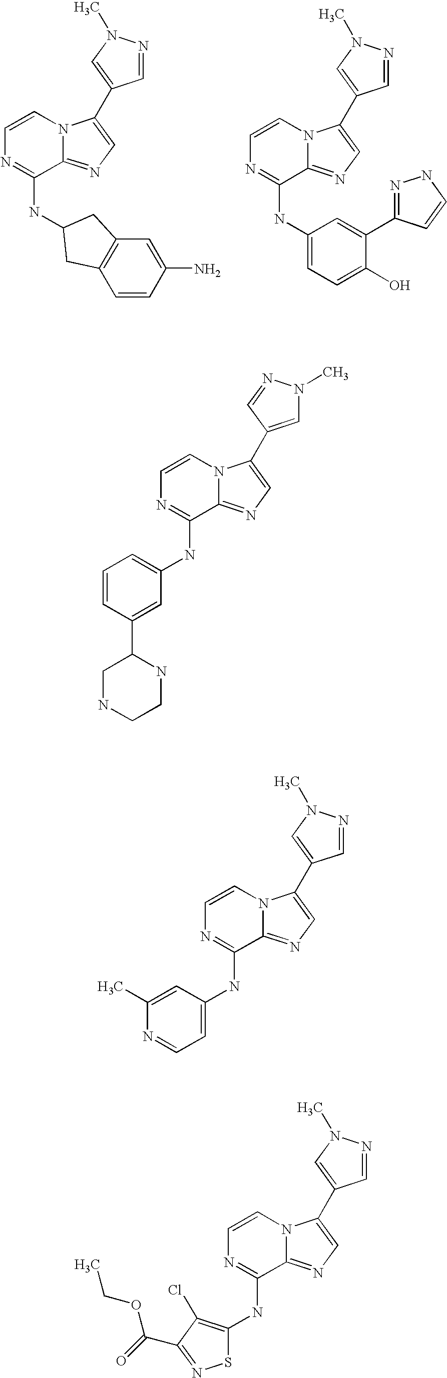 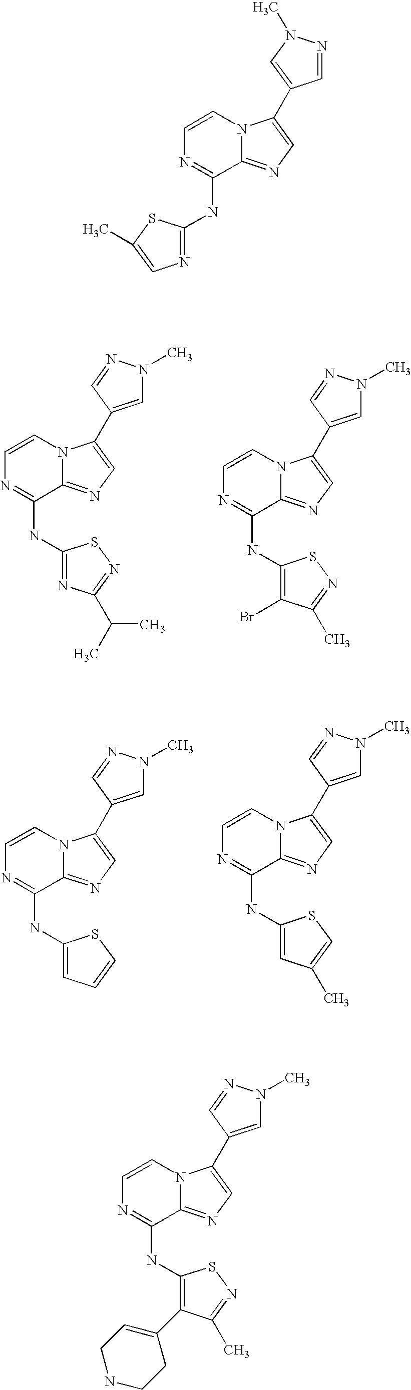 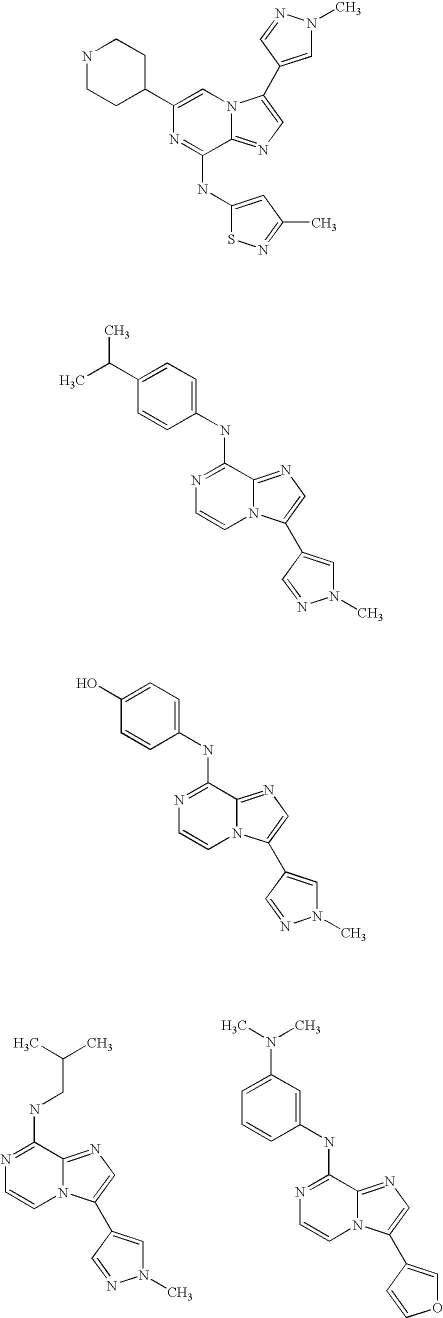 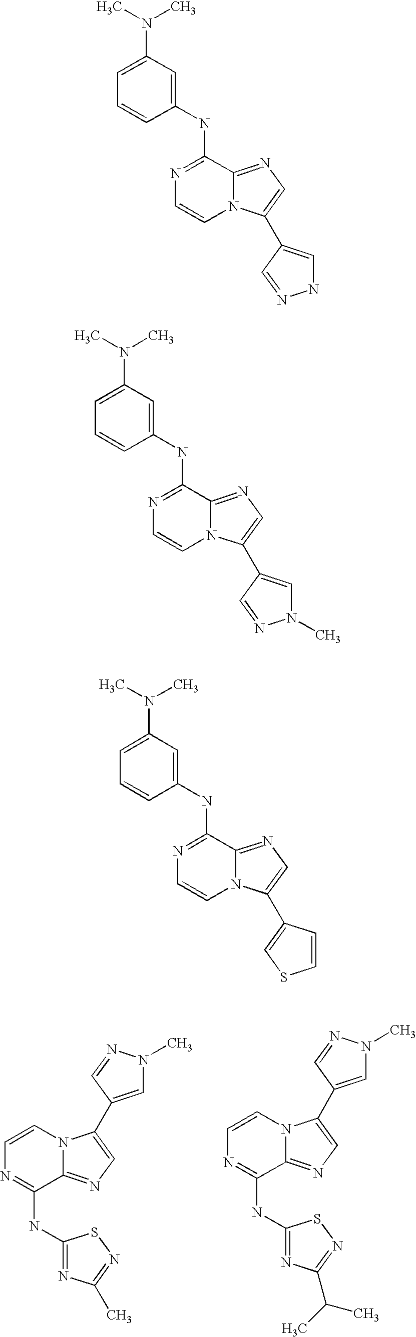 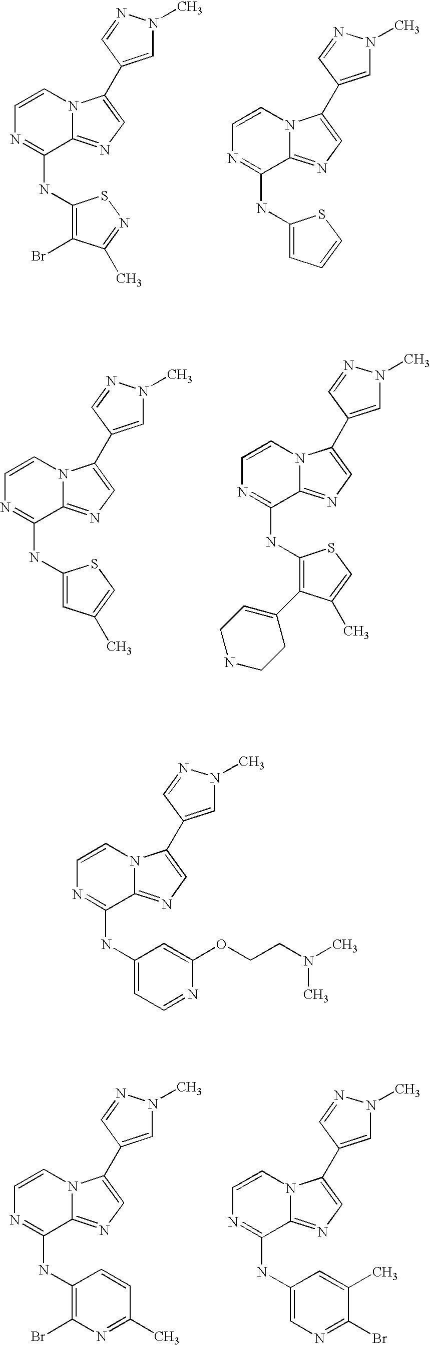 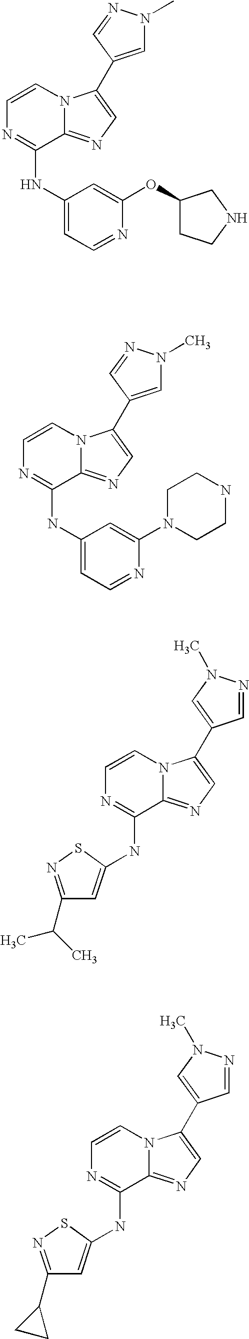 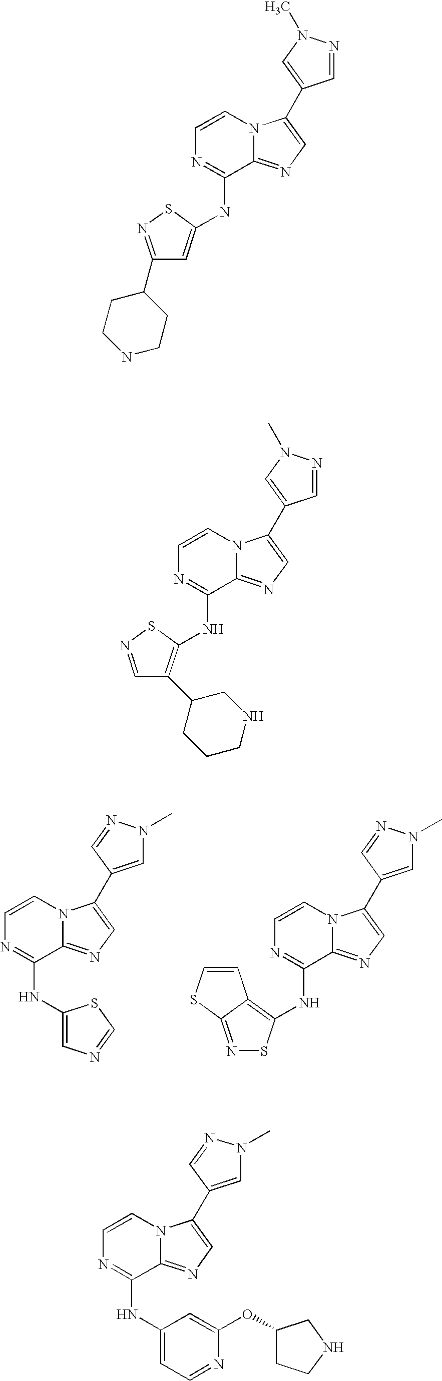 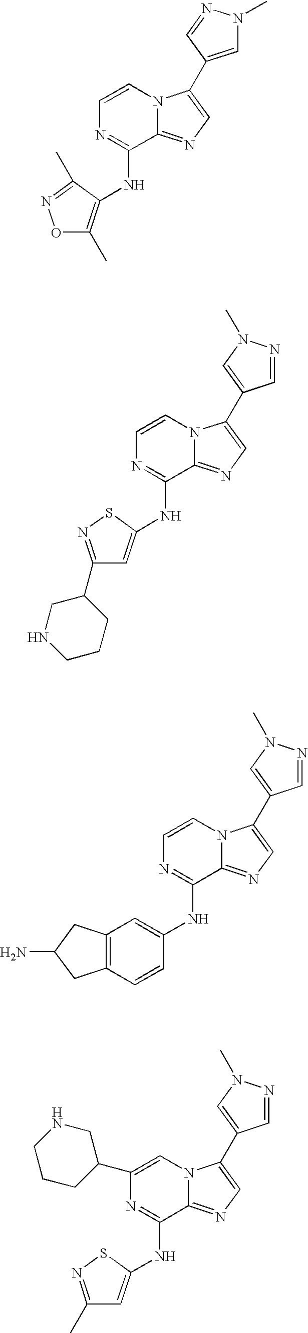 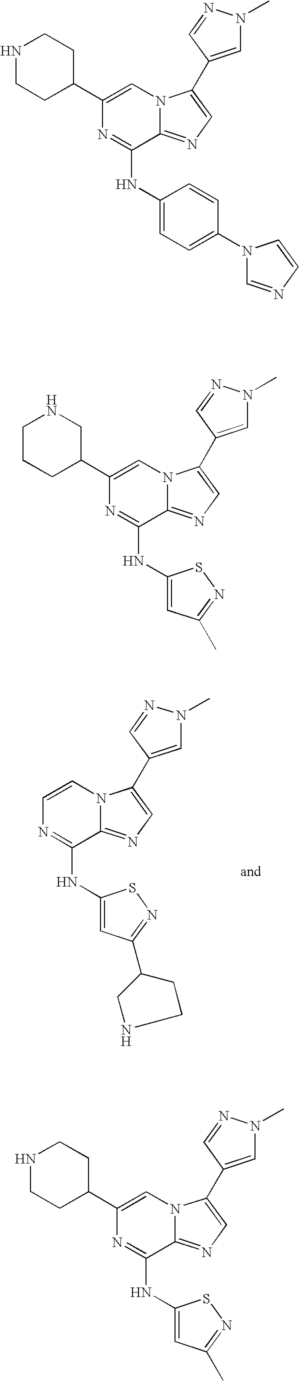
or a pharmaceutically acceptable salt, solvate, ester or prodrug thereof;
h. A compound of the formula: 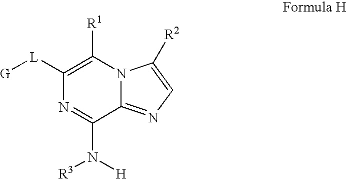
or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷ 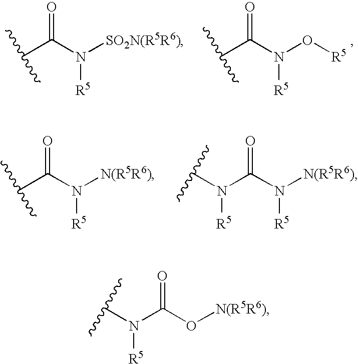
alkyl substituted with 1-6 R⁹ groups which groups can be the same or different with each R⁹ being independently selected, 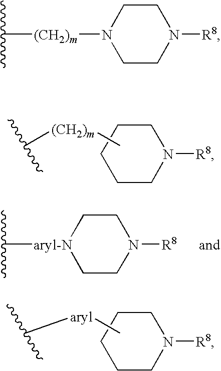
wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R³ is selected from the group consisting of H, alkyl, aryl, heteroaryl, heterocyclyl, aralkyl, heteroarylalkyl, cycloalkylalkyl, heterocyclylalkyl, 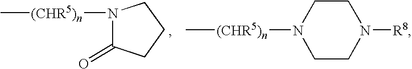
—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₘ—NR⁸, and 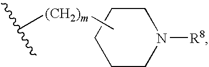
wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)NR⁵R⁶, —N(R⁵)S(O₂)R⁷, —N(R⁵)C(O)R⁷ and —N(R⁵)C(O)NR⁵R⁶;
m is 0 to 4; and
p is 0-3;
i. A compound of Formula I: 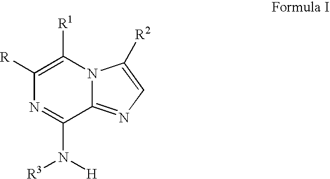
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
X is selected from the group consisting of, —NR⁵R⁶, —OR⁵, —SO—R⁵ and —SR⁵, R⁷ and R⁸ are each independently hydrogen or alkyl;
R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl, alkylN(alkyl)₂, alkylNH(alkyl), alkylN(alkenyl)2, -alkylN(alkoxyl)₂, -alkyl-SH and hydroxyalkyl, wherein each of said aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
R⁶ is selected from the group consisting of hydrogen, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl, wherein each of said aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cycloalkenylalkyl, cycloalkylalkyl, heteroarylalkyl, heterocyclenylalkyl, heterocycloalkylalkyl, heteroaryl, aryl, cycloalkenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, and heterocyclenyl;
j. A compound having the formula: 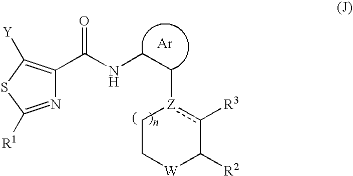
or a pharmaceutically acceptable salt, solvate, ester, prodrug or stereoisomer thereof, wherein the dashed lines indicate an optional and additional bond, and wherein:
R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;
R² is —H, -alkyl, —NH₂ or —CH₂NH₂;
R³ is —H, -alkyl, —NH₂ or —CH₂NH₂;
each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;
R⁵ is —H, -alkyl, -aryl, -heteroaryl, —NHOH;
R⁶ is —H, -alkyl or —CF₃;
R⁷ is —H, —OH, —C₁-C₆ alkyl, —O—(C₁-C₆ alkyl), or —CF₃;
R⁸ is —H, alkyl, aryl, heterocyclyl, heteroaryl or cycloalkyl;
Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen
W is —NH— or —C(R⁴)₂—, wherein both R⁴ groups and the carbon atom to which they are attached can combine to form a five to seven membered heterocyclyl or heteroaryl group;
Y is —H, -halo, -alkyl or —CN;
Z is —CR⁷— or —N—, when the optional additional bond is absent, and —C— when the optional additional bond is present;
n is an integer ranging from 0 to 2; and
(k) A compound of Formula K: 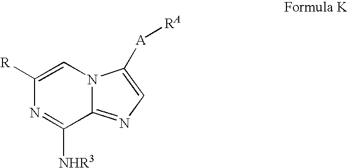
or a pharmaceutically acceptable salt, solvate, ester, prodrug or stereoisomer thereof, wherein:
R is H, halo or alkyl;
R³ is heteroaryl-X, wherein X is heterocyclylalkyl- wherein said heterocyclyl can be unsubstituted or optionally substituted with 1-4 alkyl moieties;
A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;
R^{A} is —(CH₂)₁₄-heteroaryl, 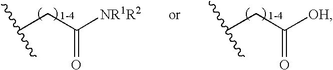
wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
R¹ and R² together with the N to which each is attached, form a heterocyclic group selected from the group consisting of 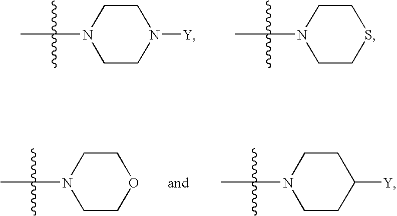
wherein
Y is alkoxyalkyl, hydroxyalkyl, dialkylaminoalkyl or alkyl, further wherein Y′ is hydroxyl;
wherein the amounts of the first compound and the second compound result in a therapeutic effect.

3. A method of inducing endoreduplication in cancer cells comprising pretreatment of cancer cells with at least one anti-mitotic agent, wherein said anti-mitotic agent is selected from the group consisting of a Taxane, paclitaxel, docetaxel, a Cenp-E Inhibitor, Abraxane, Epothilone, Monastrol, and a KSP inhibitor, Ispinesib and compounds of Formulas A-D shown in paragraphs a-d below:
a. A compound represented by the structural Formula A: 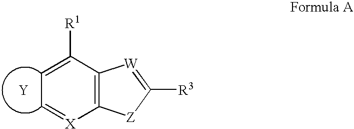
or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR C(O)OR¹¹, and —NR¹⁰)C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
b. A compound represented by the structural Formula B: 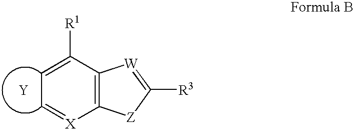
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵,
—C(O)N(R⁷)(CR⁴⁰R⁴¹)₁₋₅—OR⁷, —C(S)NR⁷(CH₂)₁₋₅NR⁴R⁵, and
—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰ NR¹⁰R¹¹, and —NR¹⁰)C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,
—C(S)R⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰,
—CN, —NR¹⁰R¹¹, —C(O)R¹⁰, —C(O)OR¹⁰, —C(O)NR¹⁰R¹¹, —CF₃, —OCF₃,
—N(R¹⁰)C(O)R¹¹, and —NR¹⁰C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰)(R¹¹), or —N(R¹⁰)C(O)R¹¹;
c. 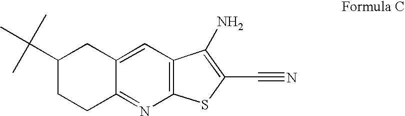
or a pharmaceutically acceptable salt, solvate, or ester thereof; and
d. A compound of Formula D 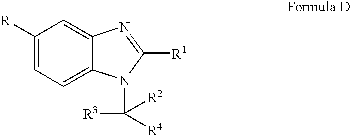
or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═OD)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
followed by administering one or more of a second compound, wherein said second compound is an aurora kinase inhibitor selected from the group consisting of compounds represented by Formulas E-K shown below in paragraphs e-k:
e. A compound represented by the structural Formula E: 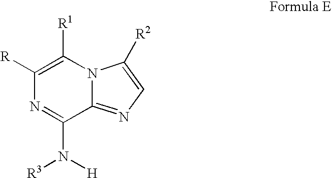
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
—N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
—NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
—CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
—S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring;
f. A compound represented by the structural formula: 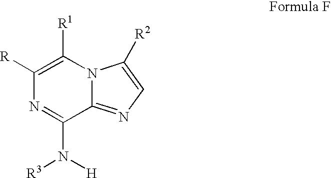
or a pharmaceutically acceptable salt, solvate, ester or prodrug thereof, wherein:
R is selected from the group consisting of H, halogen, aryl, heteroaryl, cycloalkyl, arylalkyl, heterocyclyl, heterocyclylalkyl, alkenyl, alkynyl,
—C(O)R⁷, 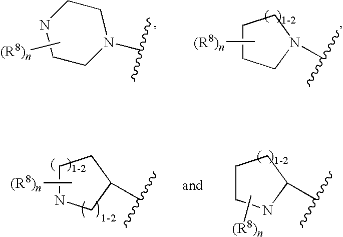
wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R¹ is H, halogen or alkyl;
R² is alkyl;
R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₘ—NR⁸, —(CHR⁵)ₙ—CH(aryl)₂, 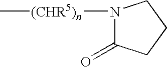
and 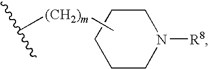
wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H or alkyl;
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and
—N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,
—S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4;
n is 1-4; and
p is 0-3;
g. A compound selected from the compounds of the formulas: 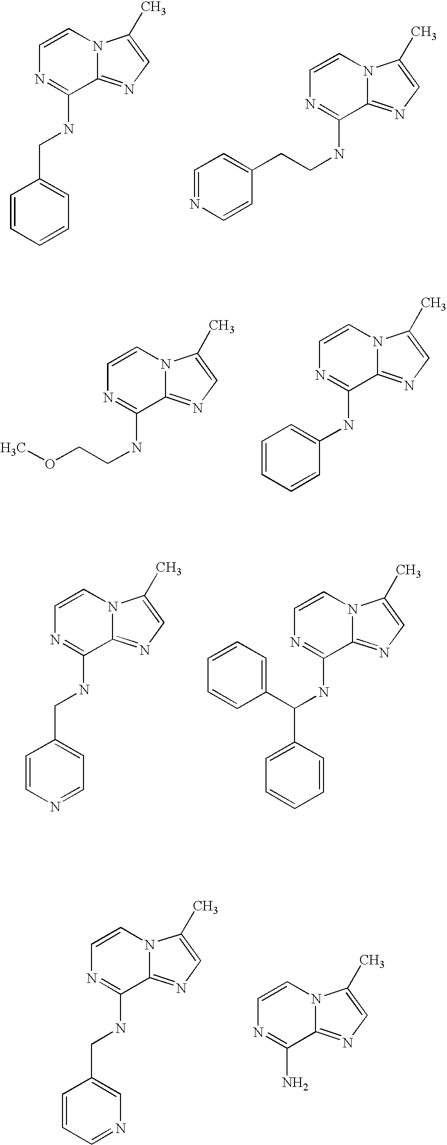 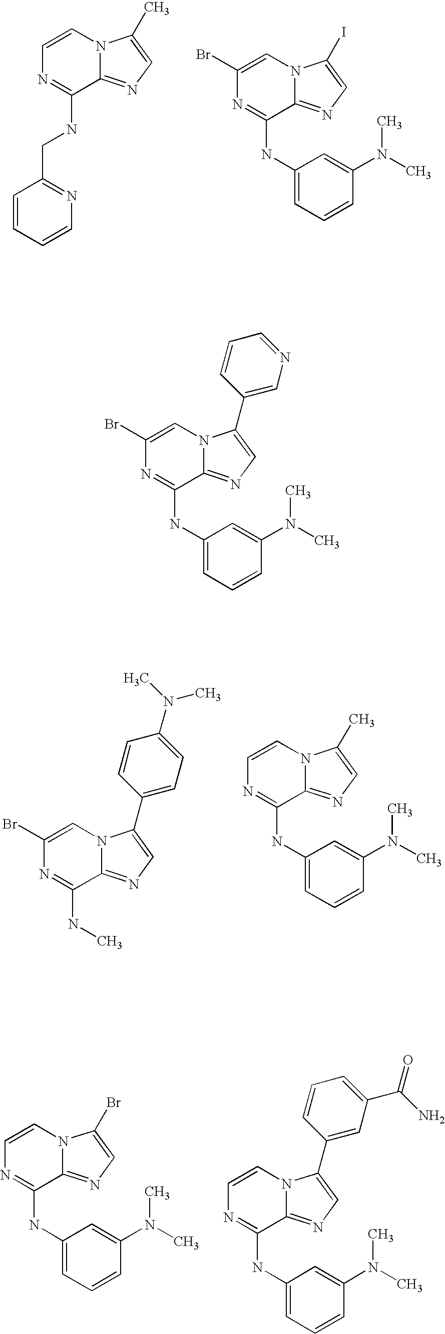 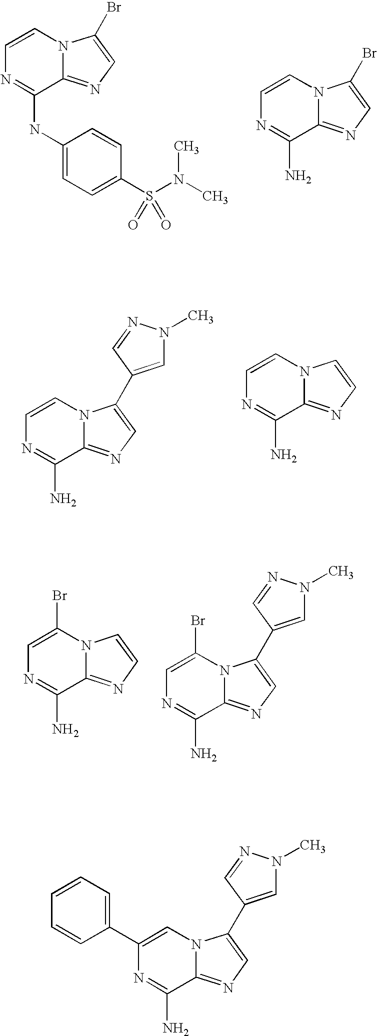 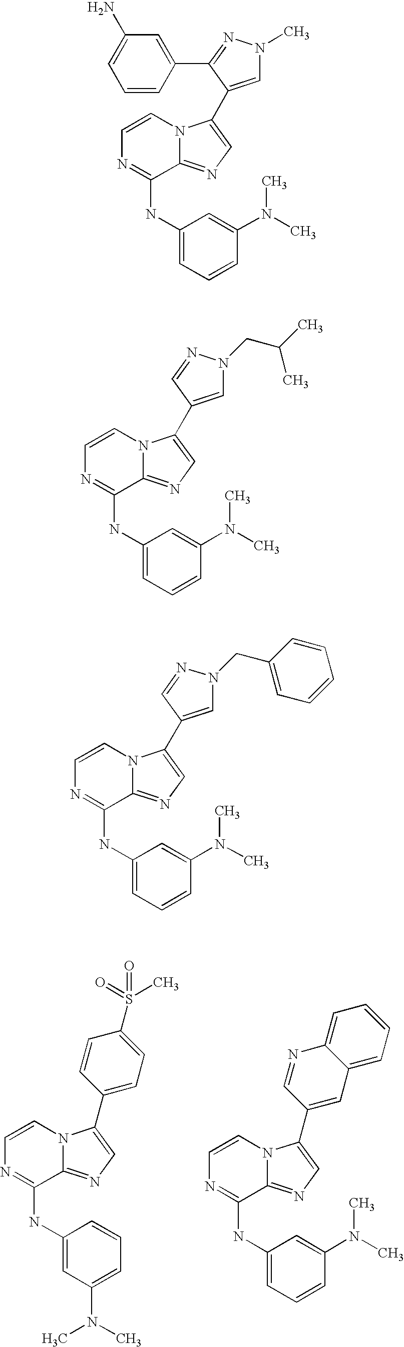 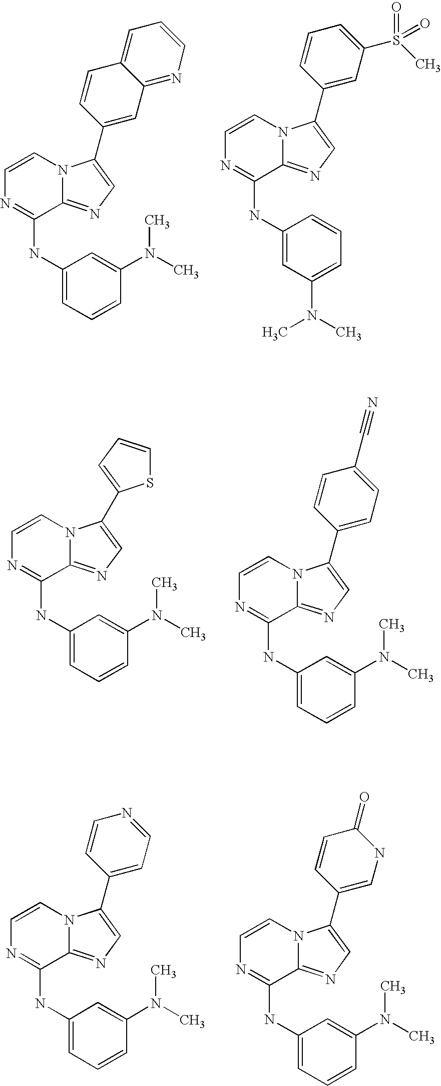 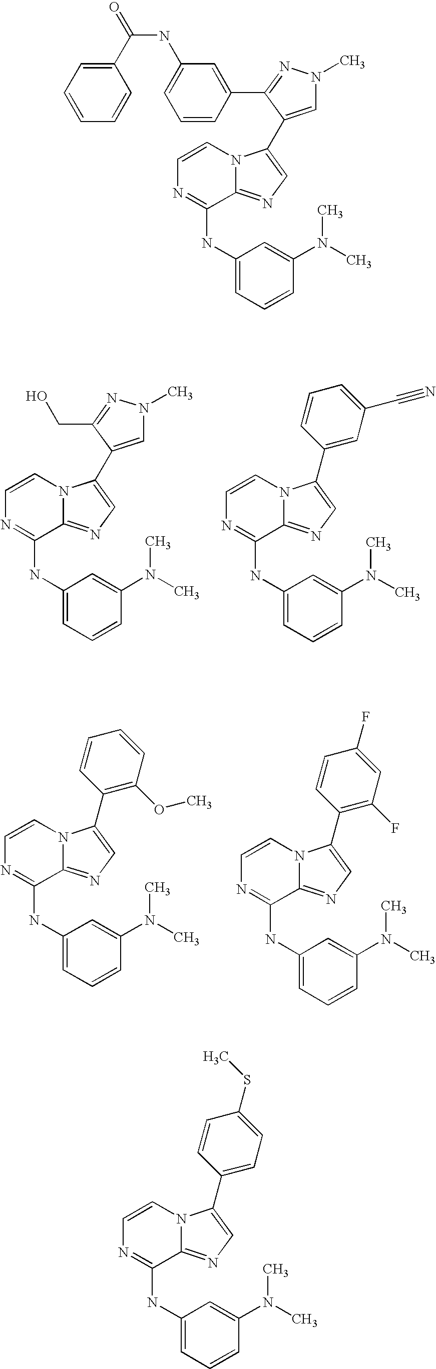 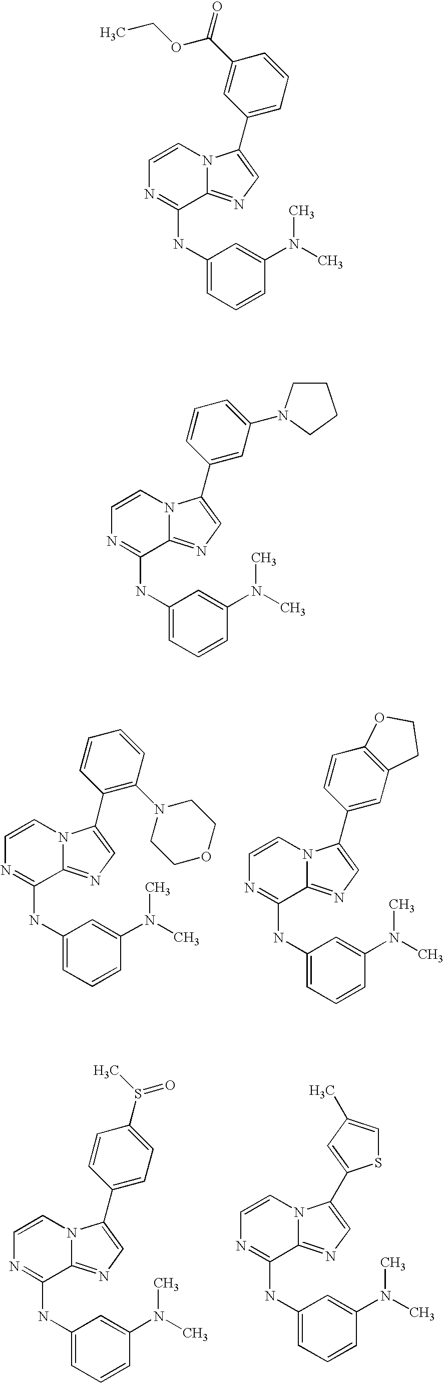 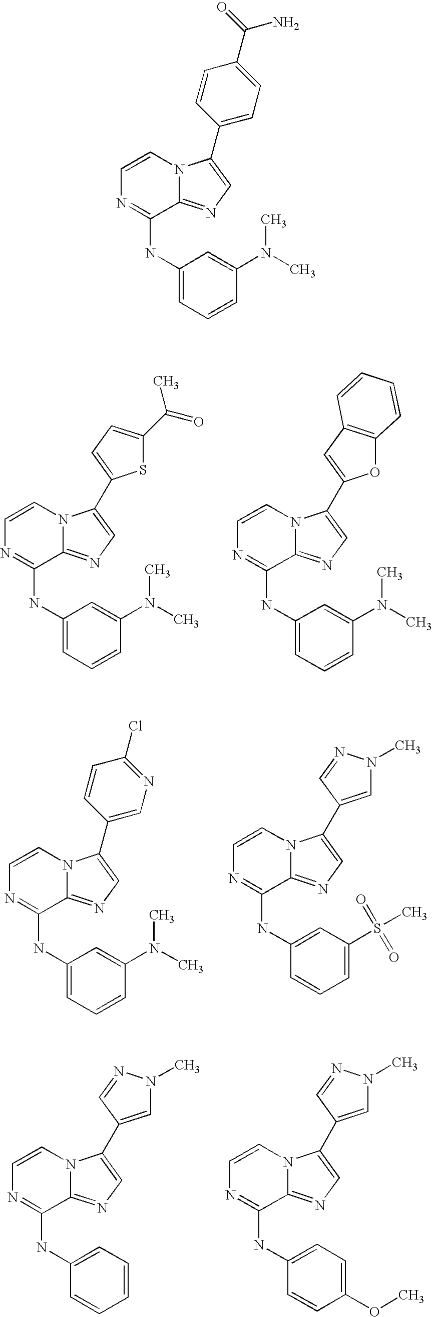 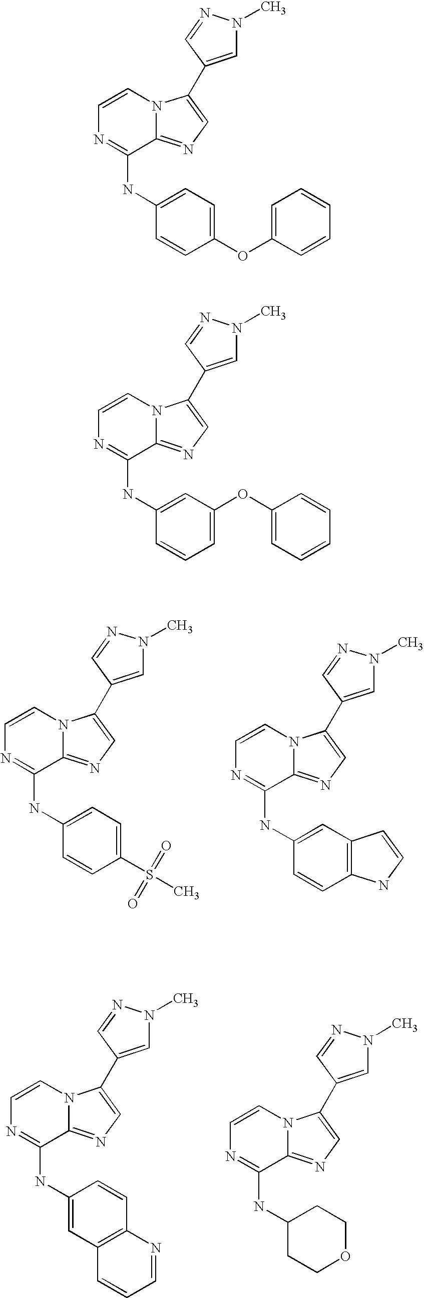 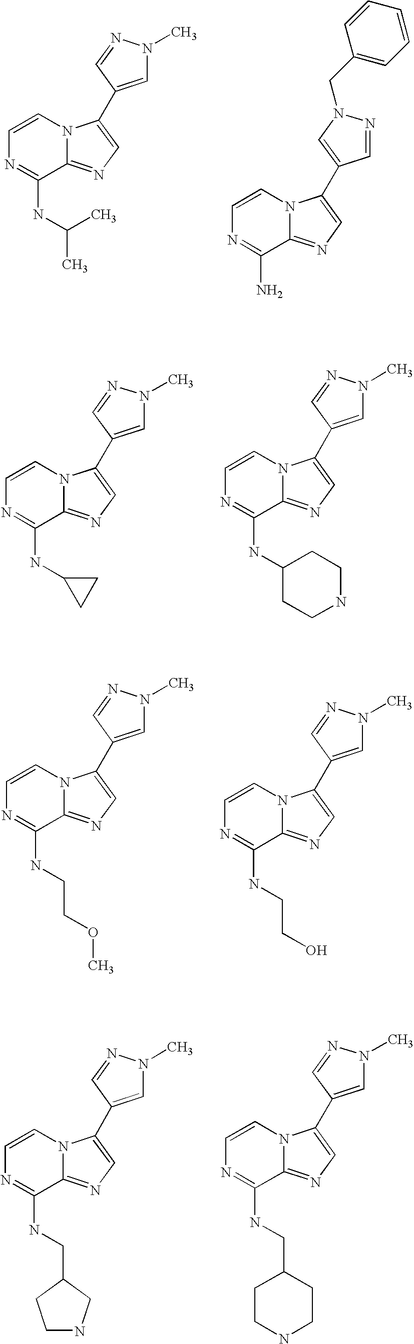 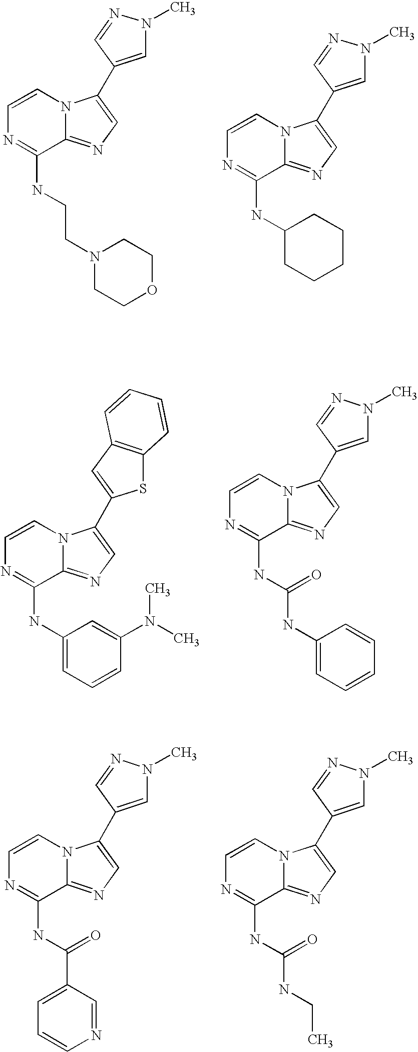 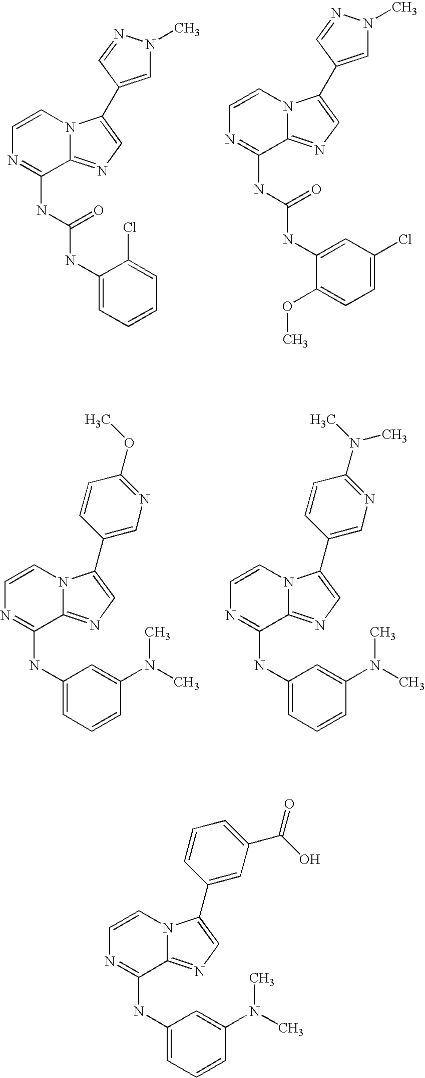 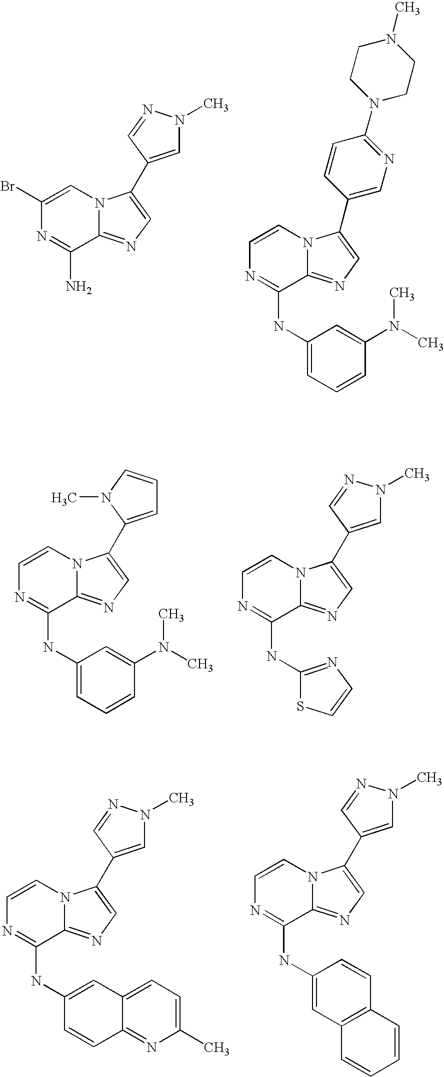 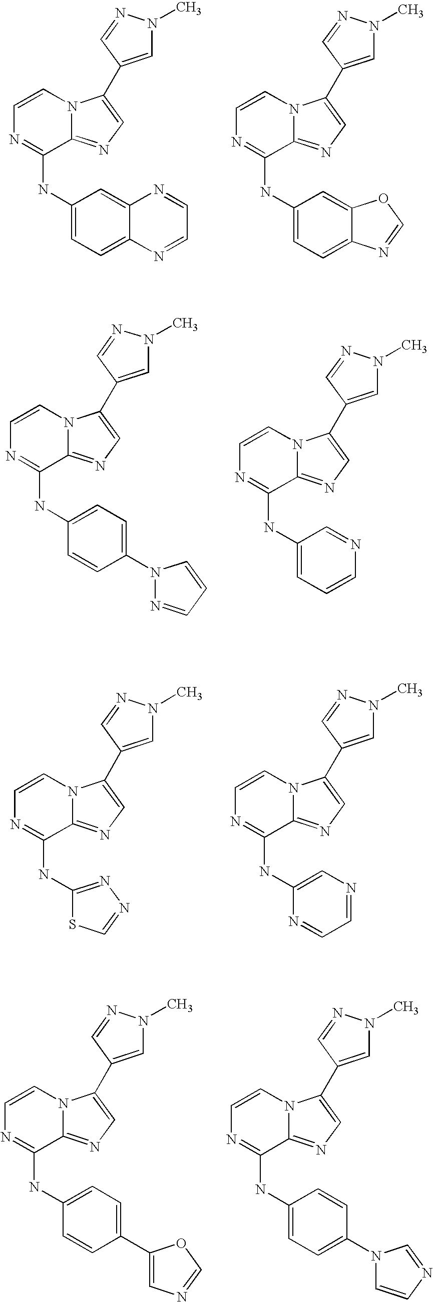 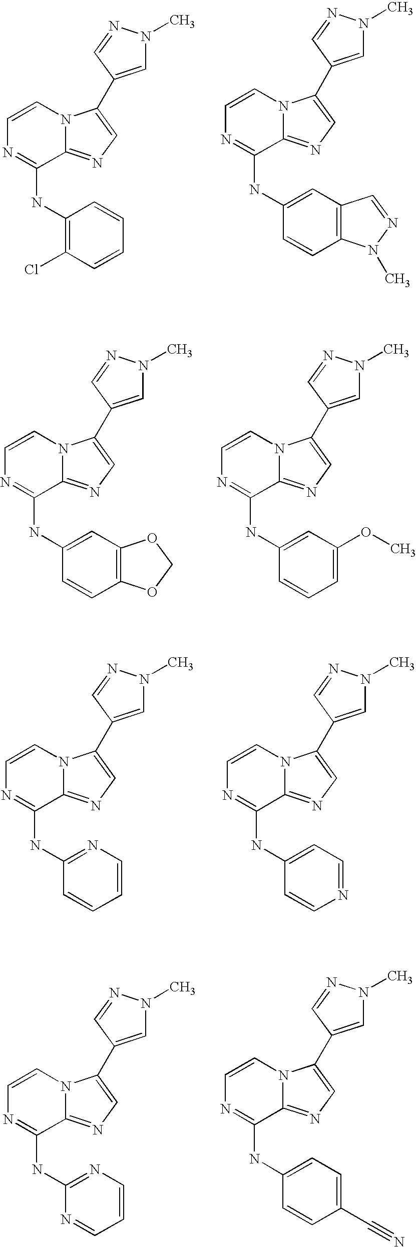 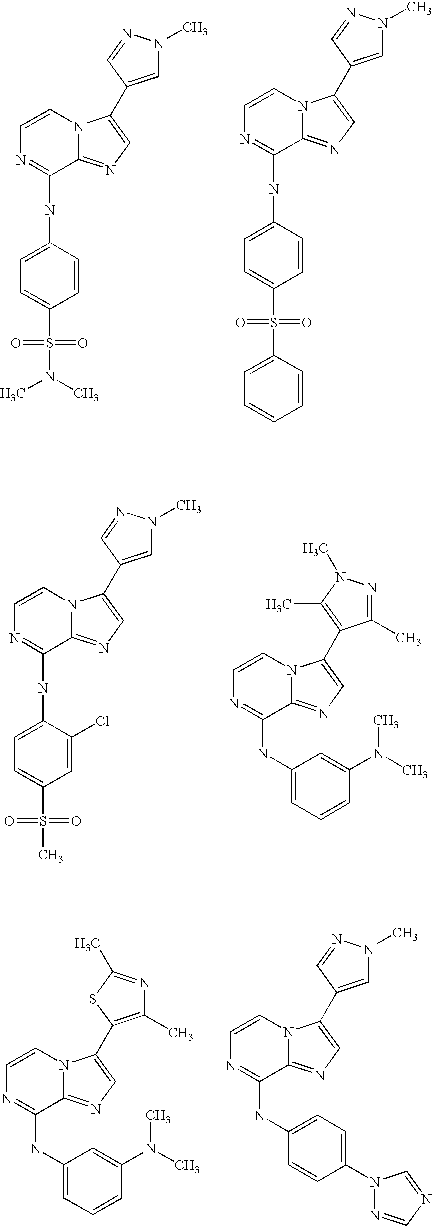 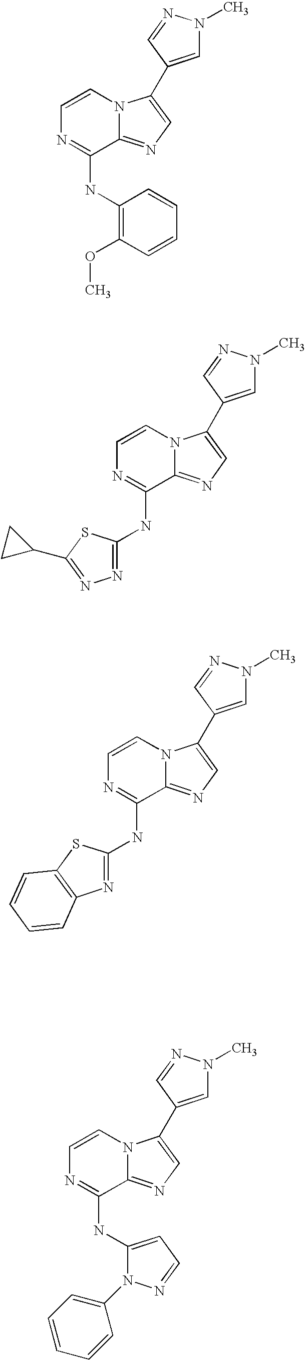 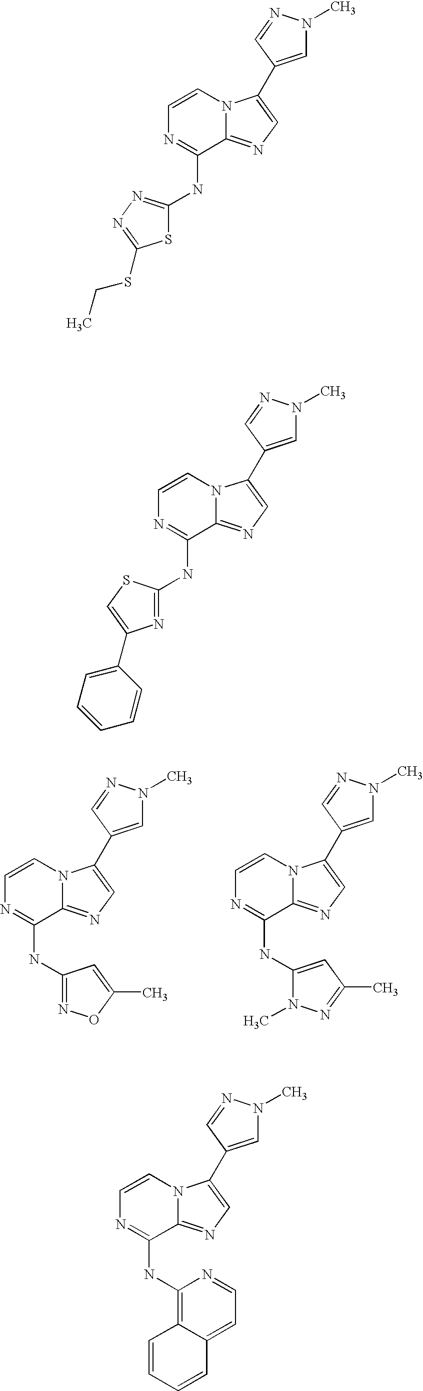 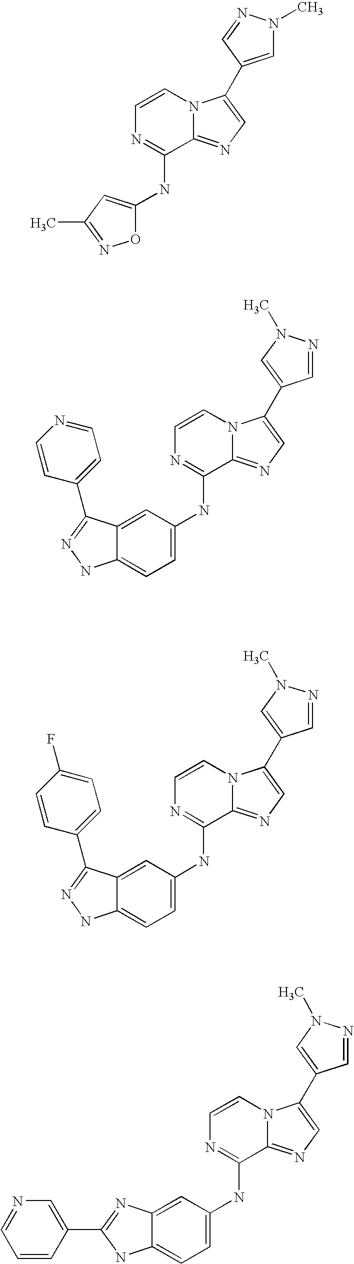 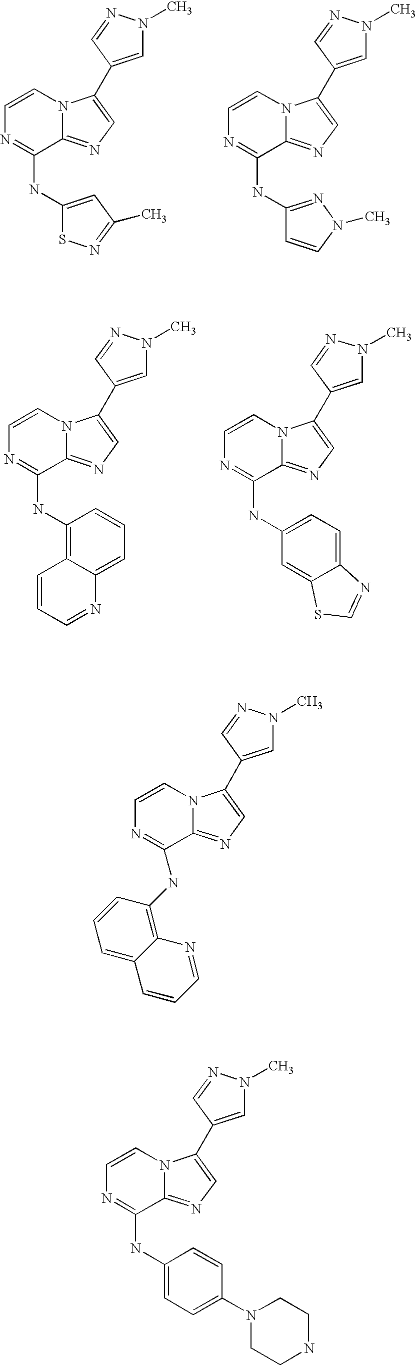 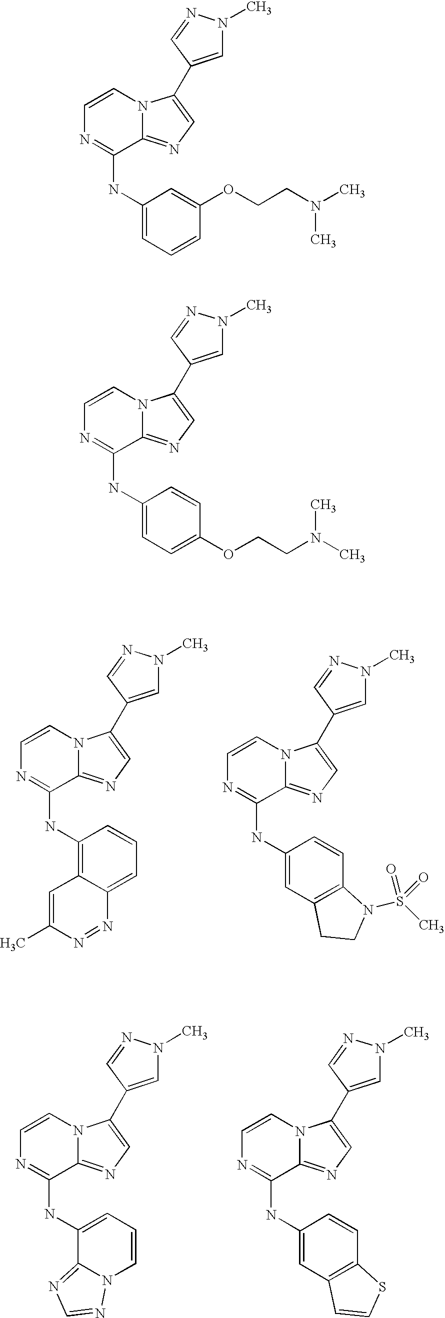 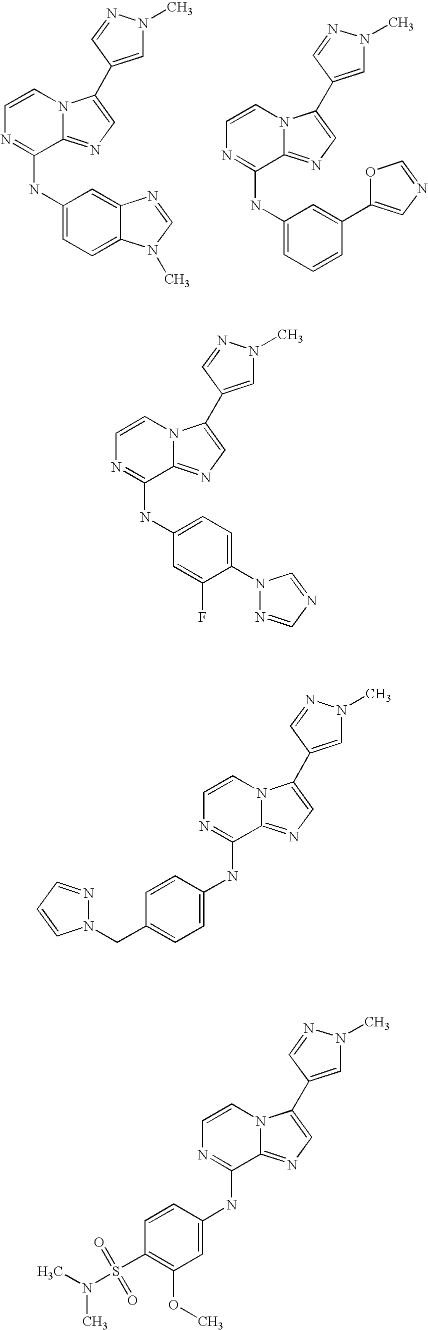 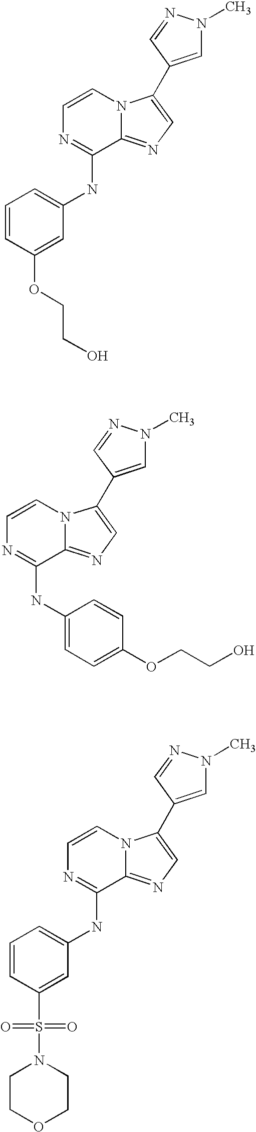 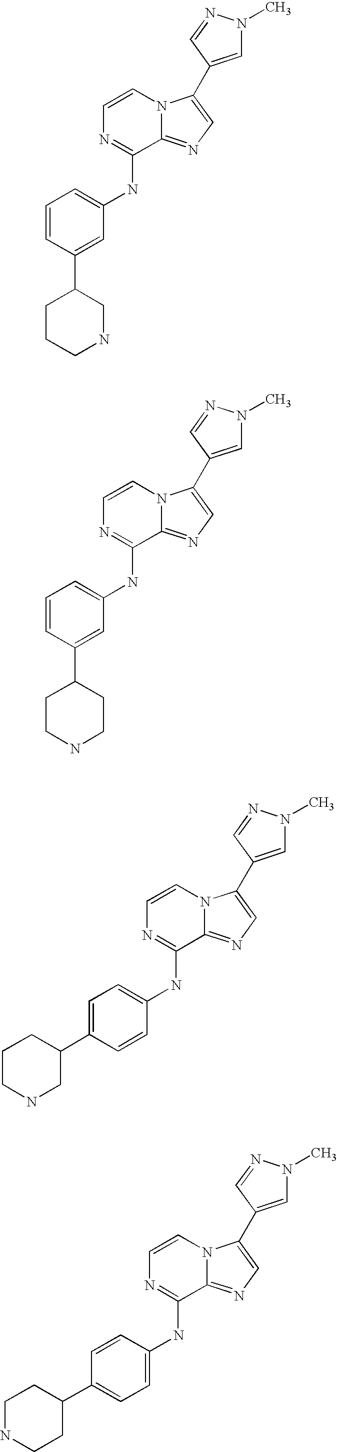 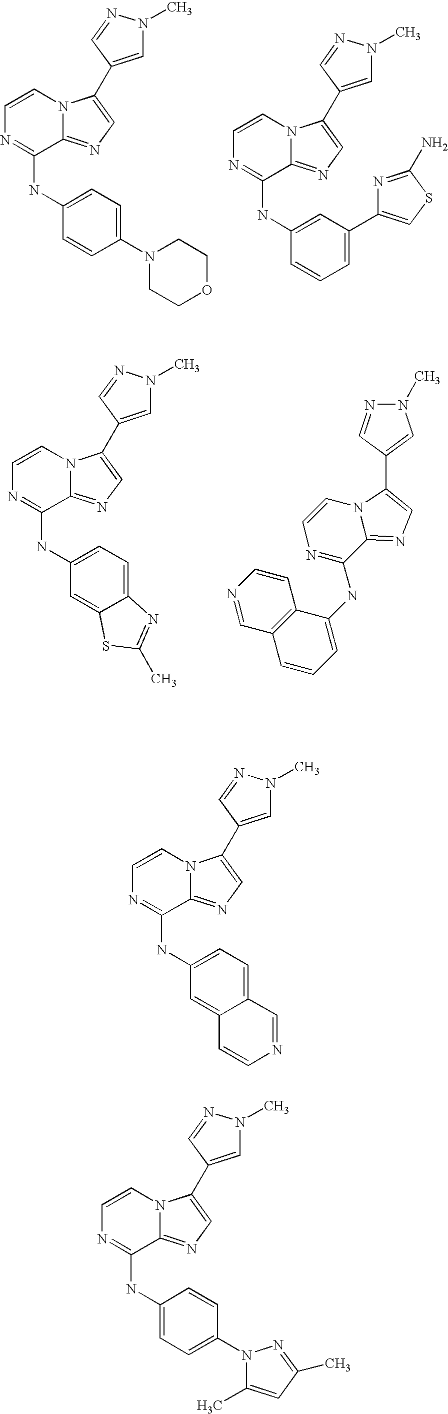 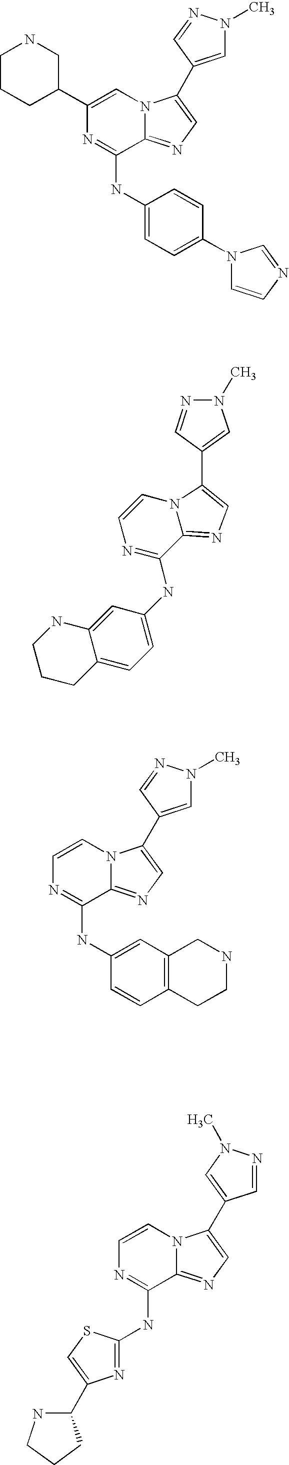 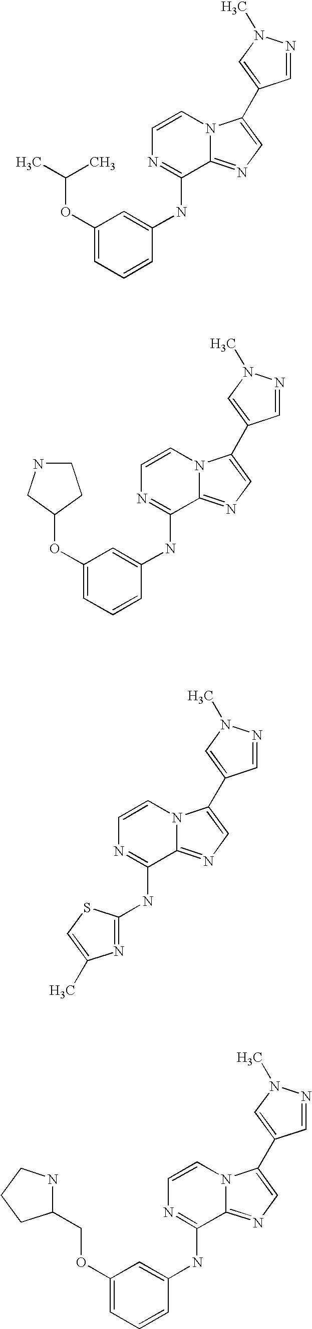 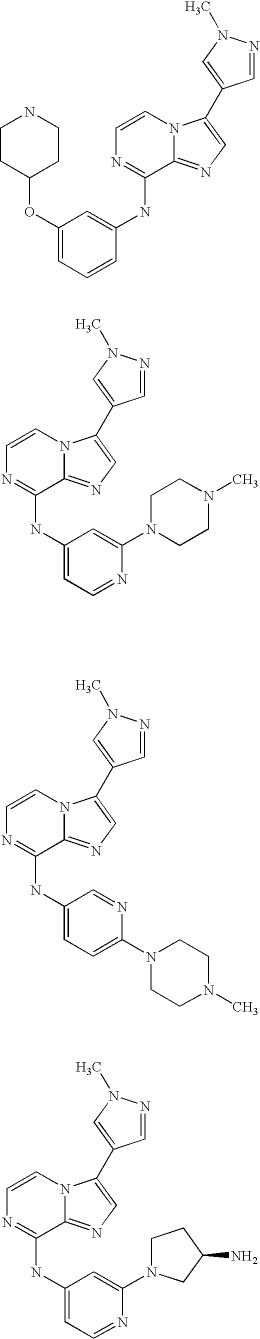 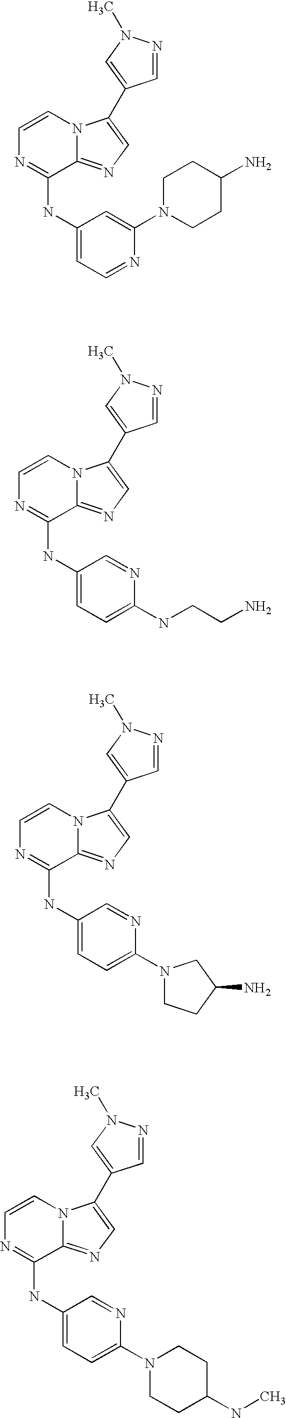 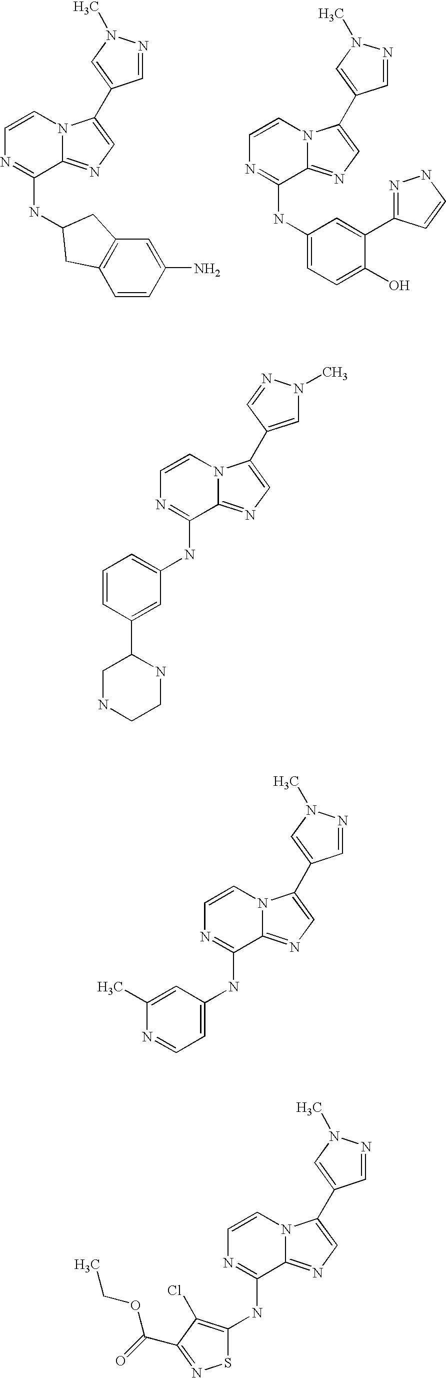 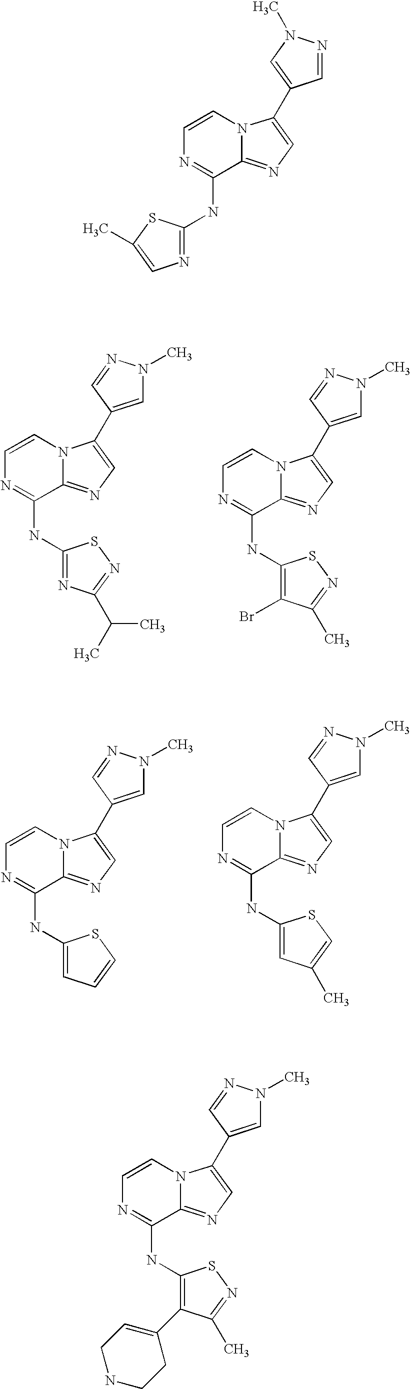 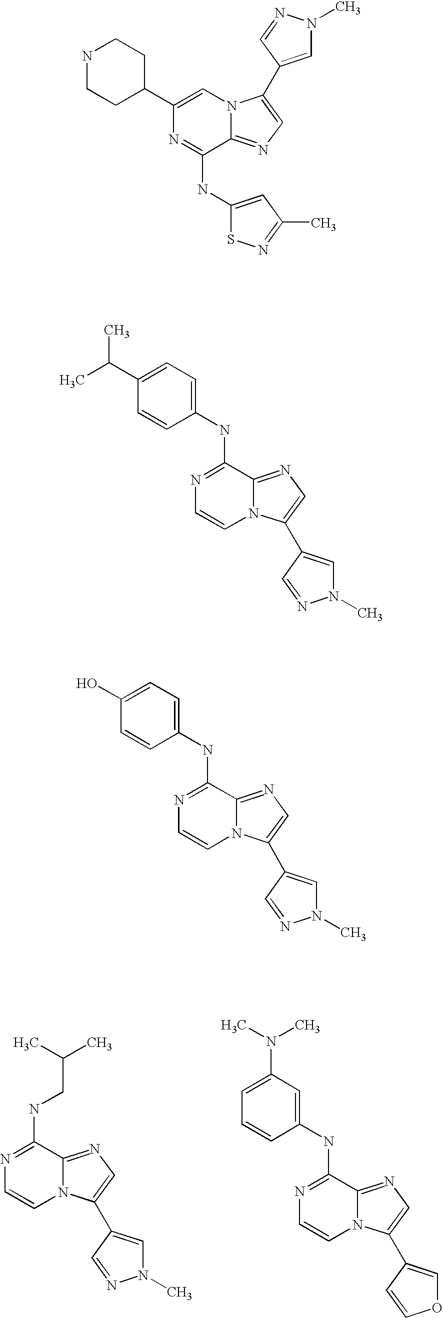 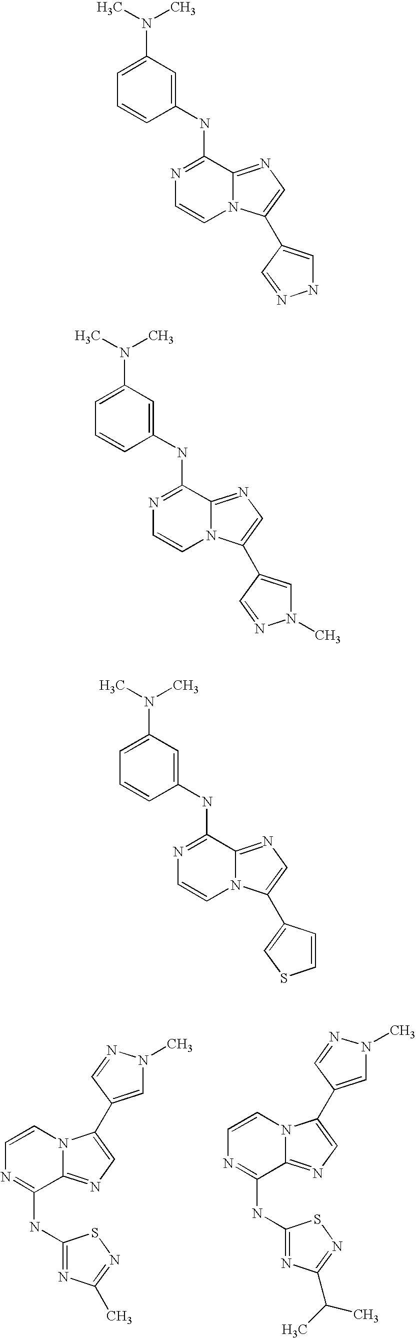 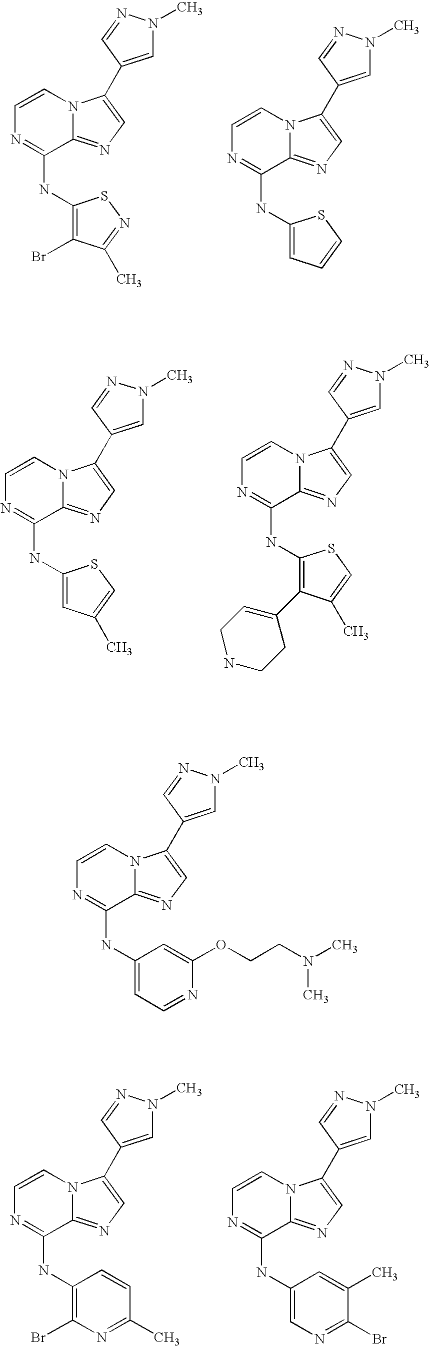 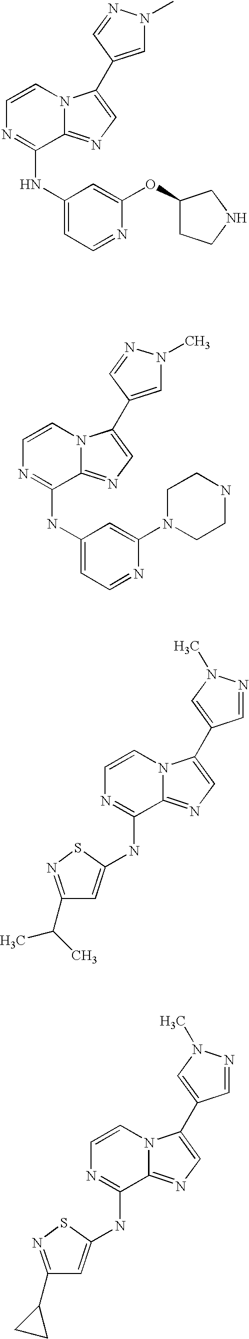 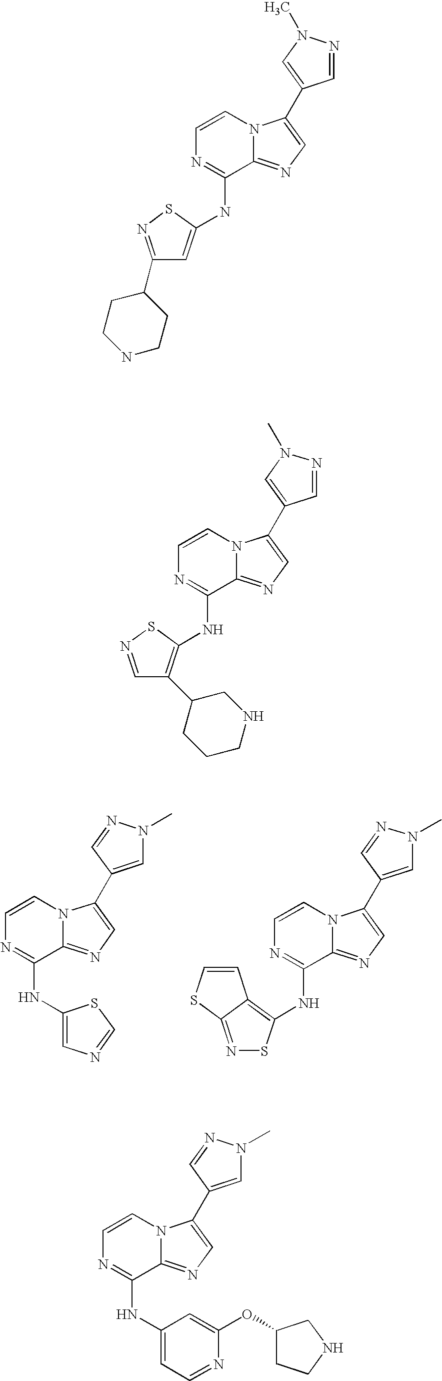 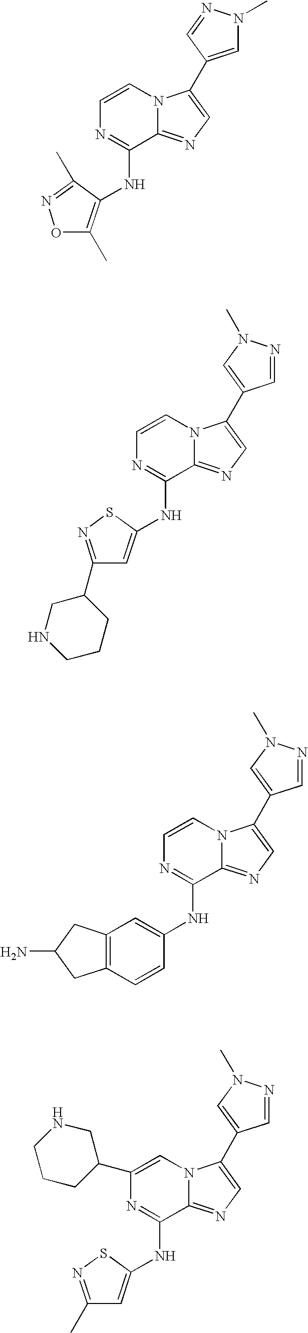 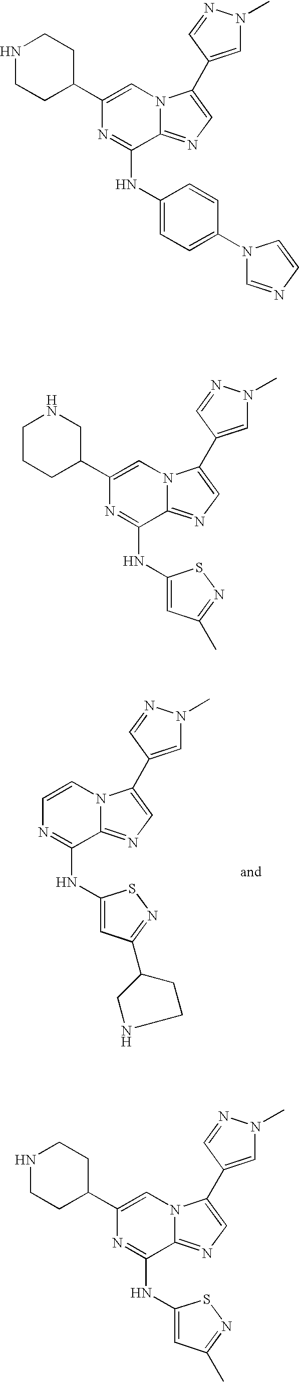
or a pharmaceutically acceptable salt, solvate, ester or prodrug thereof;
h. A compound of the formula: 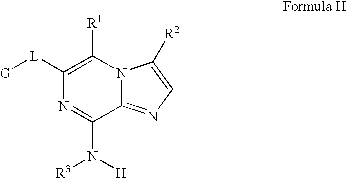
or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷, 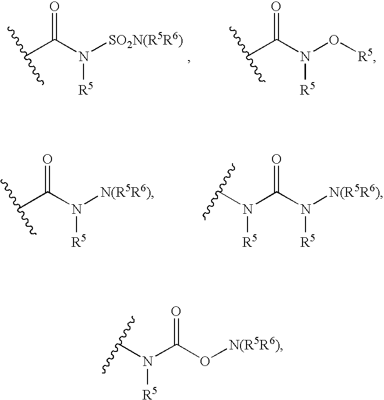
alkyl substituted with 1-6 R⁹ groups which groups can be the same or different with each R⁹ being independently selected, 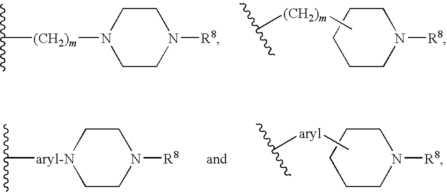
wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,
—N(R⁵)C(O)R⁷ and —N(R⁵)C(O)NR⁵R⁶;
R³ is selected from the group consisting of H, alkyl, aryl, heteroaryl, heterocyclyl, aralkyl, heteroarylalkyl, cycloalkylalkyl, heterocyclylalkyl, 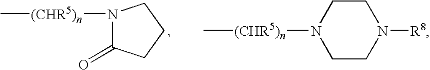
—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₙ—NR⁸, and 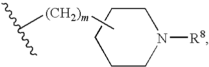
wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4; and
p is 0-3;
i. A compound of Formula I: 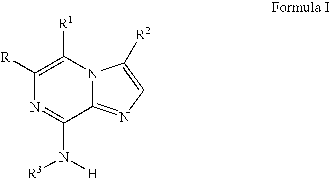
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
X is selected from the group consisting of, —NR⁵R⁶, —OR⁵, —SO—R⁵ and —SR⁵, R⁷ and R⁸ are each independently hydrogen or alkyl;
R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl, alkylN(alkyl)₂, alkylNH(alkyl), alkylN(alkenyl)2, -alkylN(alkoxyl)₂, -alkyl-SH and hydroxyalkyl, wherein each of said aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
R⁶ is selected from the group consisting of hydrogen, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkylSH, alkoxyl, —S-alkyl, hydroxyalkyl, and aminoalkyl, wherein each of said aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkylSH, alkoxyl, —S-alkyl, hydroxyalkyl, and aminoalkyl;
further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cycloalkenylalkyl, cycloalkylalkyl, heteroarylalkyl, heterocyclenylalkyl, heterocycloalkylalkyl, heteroaryl, aryl, cycloalkenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, and heterocyclenyl;
j. A compound having the formula: 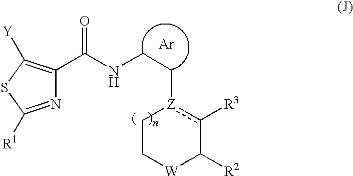
or a pharmaceutically acceptable salt, solvate, ester, prodrug or stereoisomer thereof, wherein the dashed lines indicate an optional and additional bond, and wherein:
R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;
R² is —H, -alkyl, —NH₂ or —CH₂NH₂;
R³ is —H, -alkyl, —NH₂ or —CH₂NH₂;
each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;
R⁵ is —H, -alkyl, -aryl, -heteroaryl, —NHOH;
R⁶ is —H, -alkyl or —CF₃;
R⁷ is —H, —OH, —C₁-C₆ alkyl, —O—(C₁-C₆ alkyl), or —CF₃;
R⁸ is —H, alkyl, aryl, heterocyclyl, heteroaryl or cycloalkyl;
Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen
W is —NH— or —C(R⁴)₂—, wherein both R⁴ groups and the carbon atom to which they are attached can combine to form a five to seven membered heterocyclyl or heteroaryl group;
Y is —H, -halo, -alkyl or —CN;
Z is —CR⁷— or —N—, when the optional additional bond is absent, and —C— when the optional additional bond is present;
n is an integer ranging from 0 to 2; and
(k) A compound of Formula K: 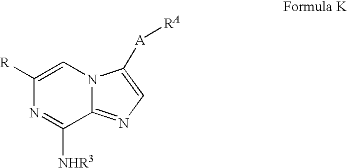
or a pharmaceutically acceptable salt, solvate, ester, prodrug or stereoisomer thereof, wherein:
R is H, halo or alkyl;
R³ is heteroaryl-X, wherein X is heterocyclylalkyl- wherein said heterocyclyl can be unsubstituted or optionally substituted with 1-4 alkyl moieties;
A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;
R^{A} is —(CH₂)₁₋₄-heteroaryl, 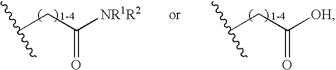
wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
R¹ and R² together with the N to which each is attached, form a heterocyclic group selected from the group consisting of 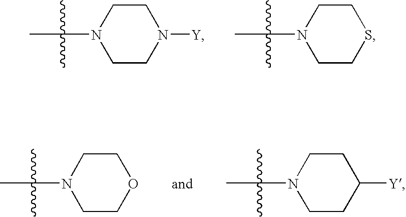
wherein
Y is alkoxyalkyl, hydroxyalkyl, dialkylaminoalkyl or alkyl, further wherein Y′ is hydroxyl.

4. The method of claim 3, wherein endoreduplication in cancer cells results in death of the cancer cell, wherein said cancer cell is selected from the group consisting of tumor of the bladder, breast (including BRCA-mutated breast cancer), colorectal, colon, kidney, liver, lung (including small cell lung cancer and non-small cell lung cancer), head and neck, esophagus, bladder, gall bladder, ovary, pancreas, stomach, cervix, thyroid, prostate, and skin, including squamous cell carcinoma;
leukemia, acute lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma and Burkett's lymphoma;
chronic lymphocytic leukemia (“CLL”),
acute and chronic myelogenous leukemia, myelodysplastic syndrome and promyelocytic leukemia;
fibrosarcoma, rhabdomyosarcoma;
mantle cell lymphoma, myeloma;
astrocytoma, neuroblastoma, glioblastoma, malignant glial tumors, hepatocellular carcinoma, gastrointestinal stromal tumors (“GIST”) and schwannomas;
melanoma, multiple myeloma, seminoma, teratocarcinoma, osteosarcoma, xenoderoma pigmentosum, keratoctanthoma, thyroid follicular cancer and Kaposi's sarcoma.

5. The method of claim 4, wherein said cancer cell is selected from the group consisting of colon cancer cell, breast cancer cell, lung cancer cell, prostrate cancer cell, and ovarian cancer cell.

6. The method of claim 3, wherein said pretreatment of cancer cells with at least one of said anti-mitotic agents is for at least about 12-24 hours.

7. The method of claim 6, wherein said pretreatment of cancer cells with at least one of said anti-mitotic agents is for at least about 16 hours.

8. The method of claim 6, wherein after said pretreatment of cancer cells with said antimotic agent, the cancer cells are exposed to at least one aurora kinase inhibitor for about 2-12 hours.

9. The method of claim 7, wherein after said pretreatment of cancer cells with said antimotic agent, the cancer cells are exposed to at least one aurora kinase inhibitor for about 4 hours.

10. A method of retarding the growth of a tumor in vivo comprising the steps of (a) first administering to tumor in vivo at least one anti-mitotic agent selected from the group consisting of a taxane, paclitaxel, docetaxel, a Cenp-E Inhibitor, Abraxane, Epothilone, Monastrol, a KSP inhibitor, Ispinesib and compounds represented by Formulas A-D shown in paragraphs a-d below:
a. A compound represented by the structural Formula A: 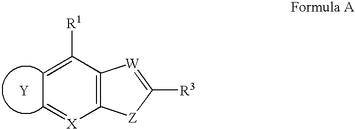
or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ; alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —OR⁷, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰C(O)OR¹¹, and —NR¹⁰C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
b. A compound represented by the structural Formula B: 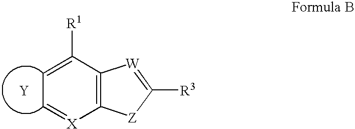
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵,
—C(O)N(R⁷)(CR⁴⁰R⁴¹)₁₋₅—OR⁷, —C(S)NR⁷(CH₂)₁₋₅NR⁴R⁵, and
—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl,
—OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,
—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰,
—CN, —NR¹⁰R¹¹, —C(O)R¹⁰, —C(O)OR¹⁰, —C(O)NR¹⁰R¹¹, —CF₃, —OCF₃,
—N(R¹⁰)C(O)R¹¹, and —NR¹⁰C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰ (R¹¹), or —N(R¹⁰)C(O)R¹¹;
c. 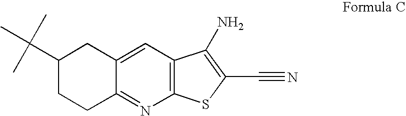
or a pharmaceutically acceptable salt, solvate, or ester thereof; and
d. A compound of Formula D 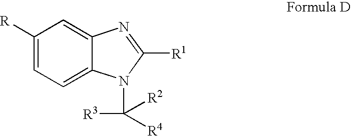
or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl, —C(═O)heteroaryl; —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
and (b) subsequently administering to said tumor at least one aurora kinase inhibitor, wherein said aurora kinase inhibitor is selected from the group consisting of a compound represented by Formulas E-K shown below in paragraphs e-k:
e. A compound represented by the structural Formula E: 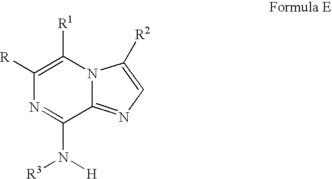
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
—N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
—NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
—CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
—S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring;
f. A compound represented by the structural formula: 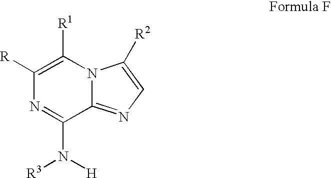
or a pharmaceutically acceptable salt, solvate, ester or prodrug thereof, wherein:
R is selected from the group consisting of H, halogen, aryl, heteroaryl, cycloalkyl, arylalkyl, heterocyclyl, heterocyclylalkyl, alkenyl, alkynyl,
—C(O)R⁷, 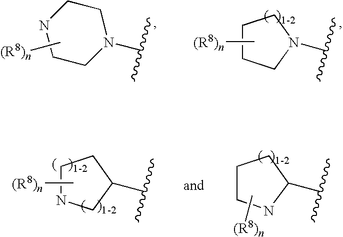
wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R¹ is H, halogen or alkyl;
R² is alkyl;
R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₙ—NR⁸, —(CHR⁵)ₙ—CH(aryl)₂, 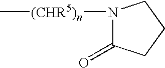
and 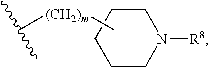
wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H or alkyl;
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and
—N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,
—S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4;
n is 1-4; and
p is 0-3;
g. A compound selected from the compounds of the formulas: 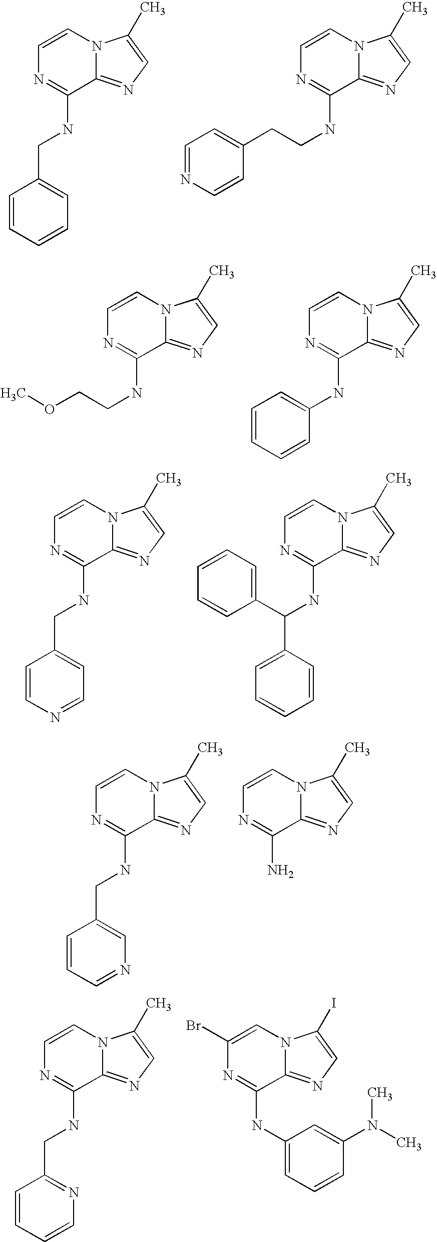 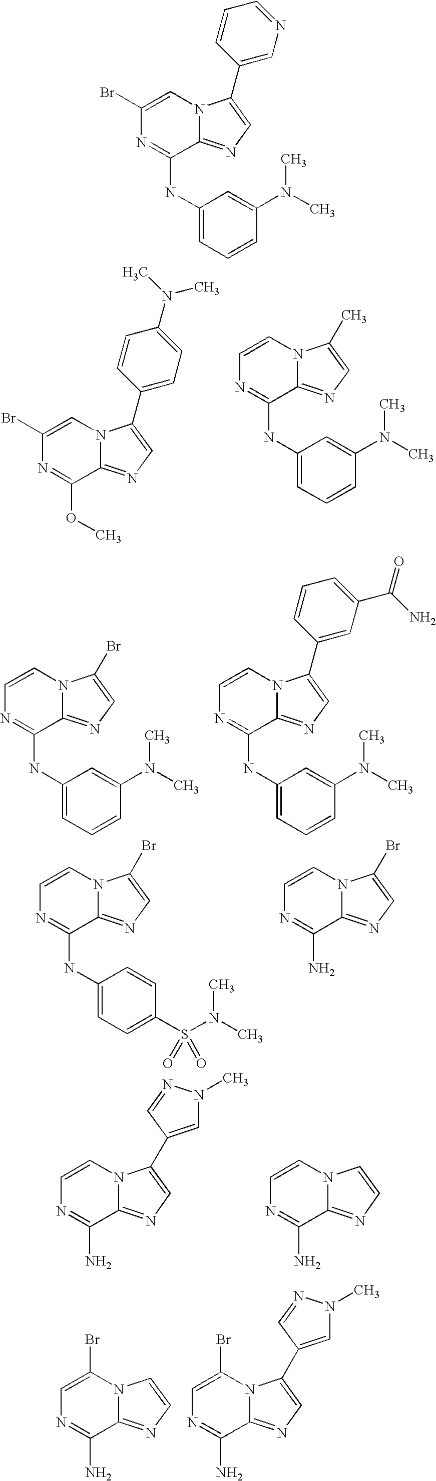 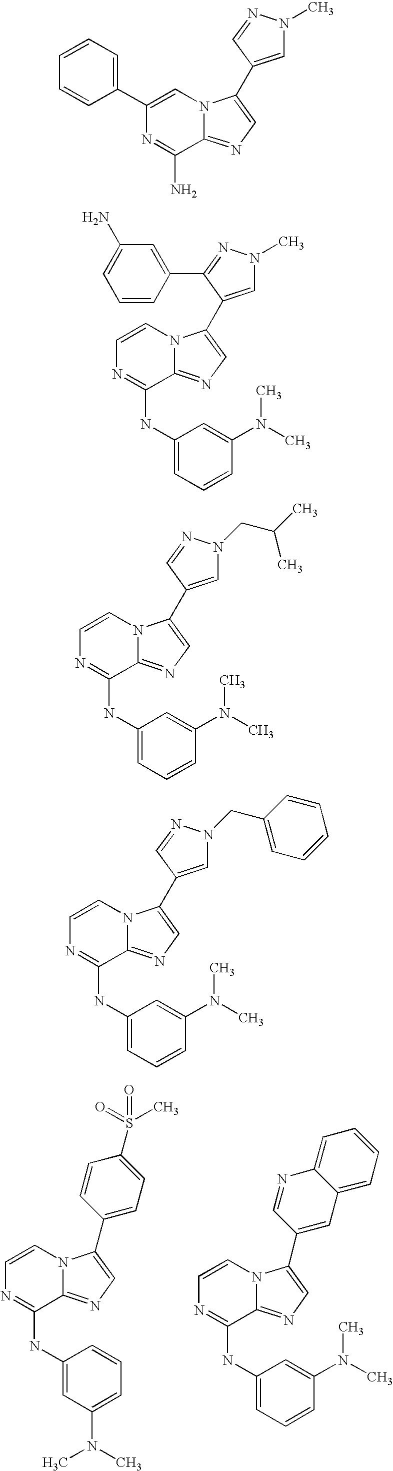 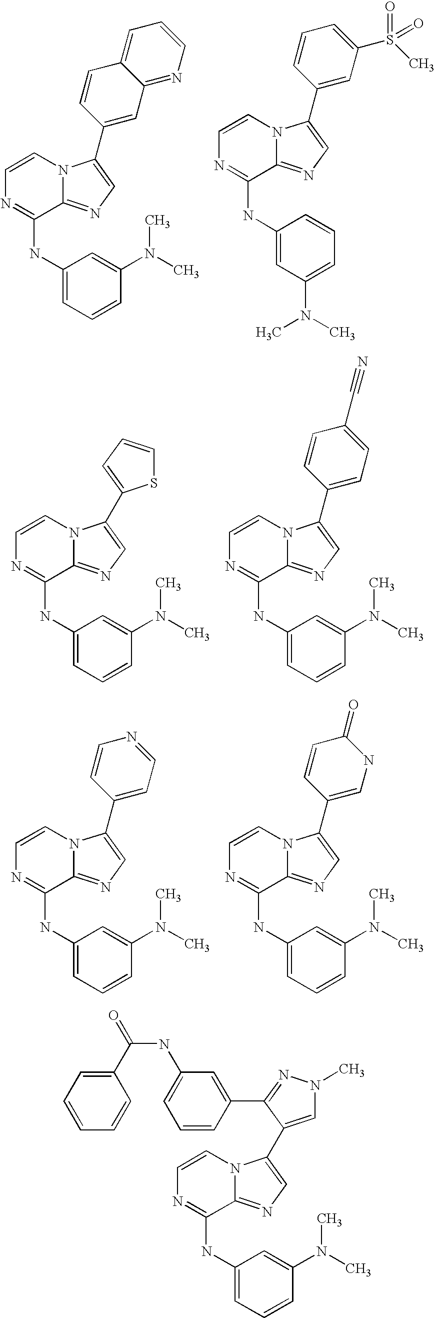 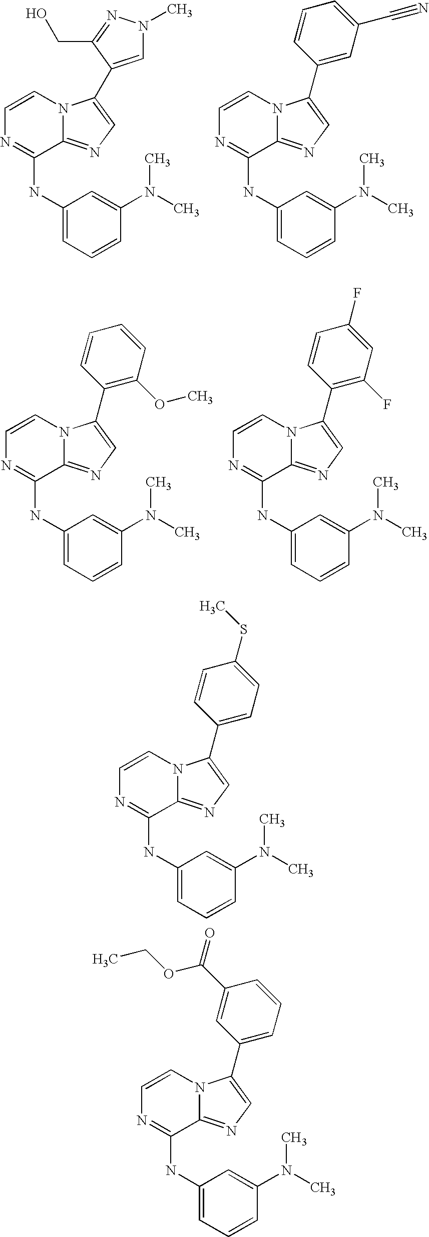 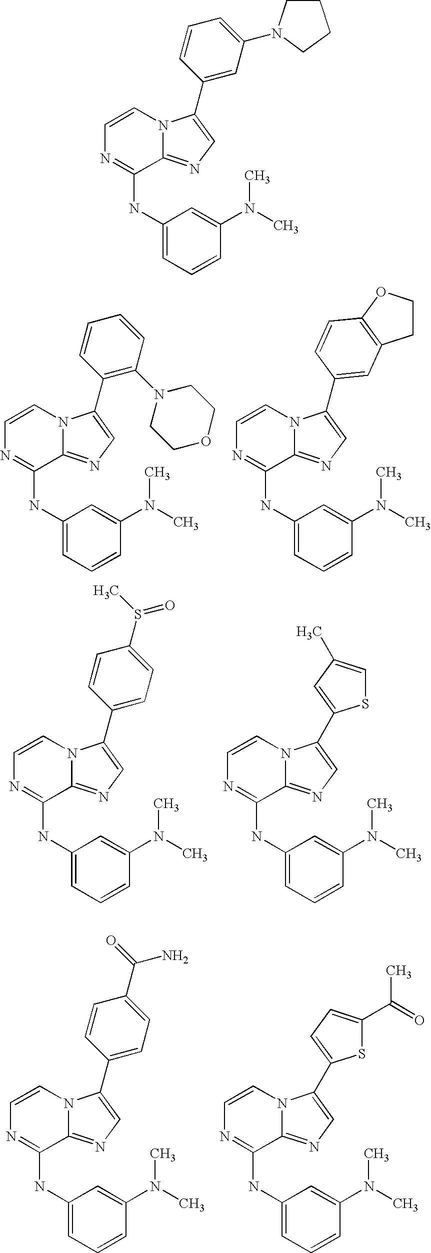 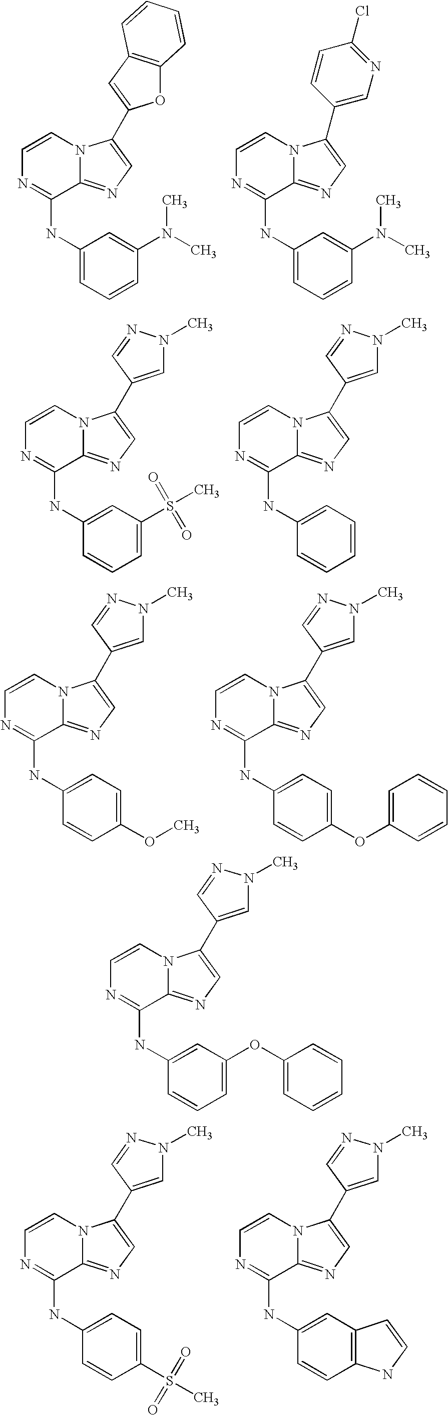 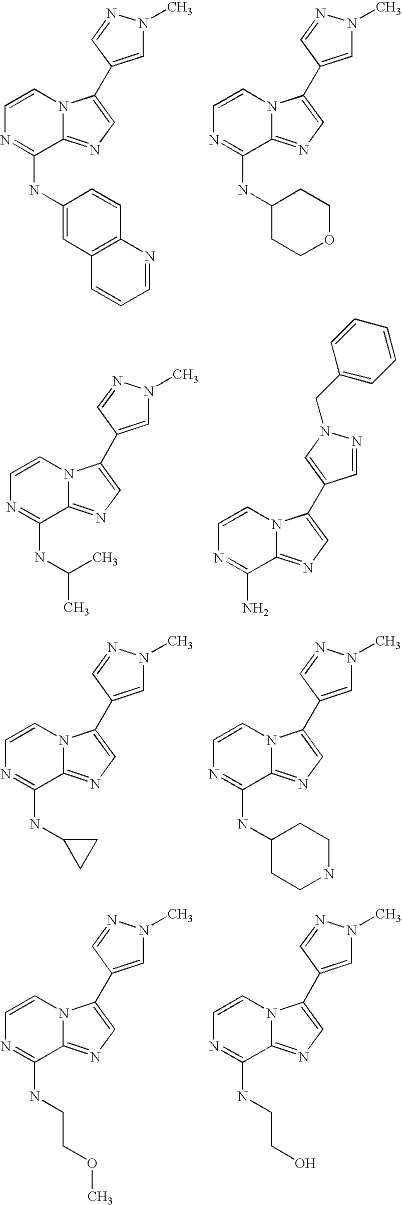 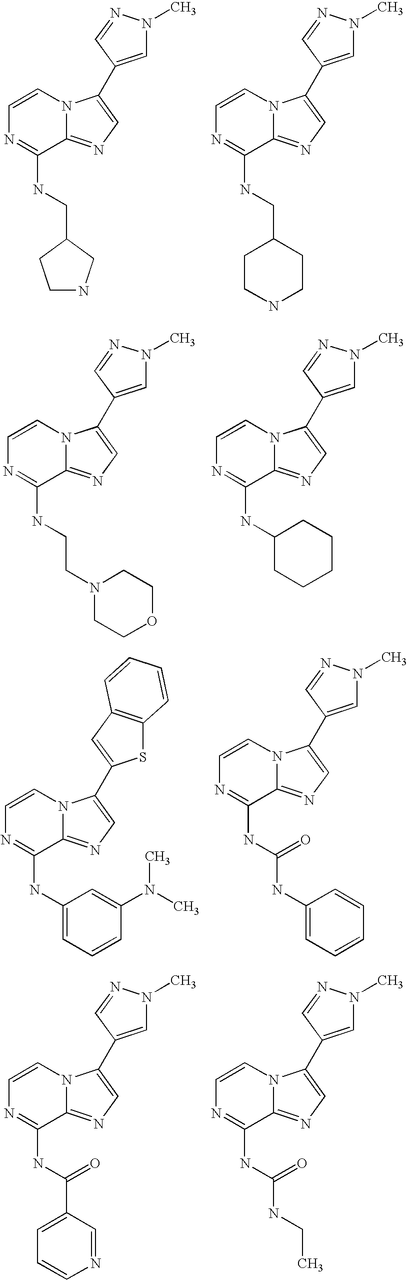 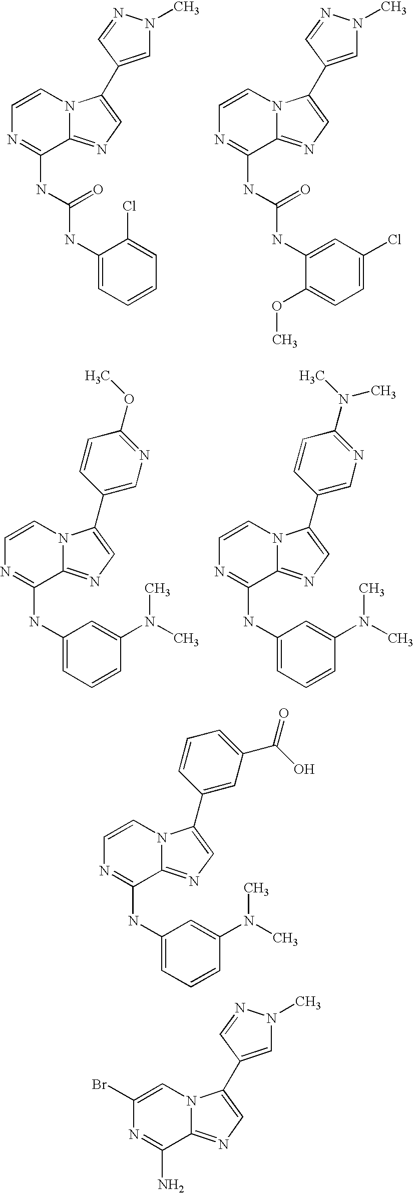 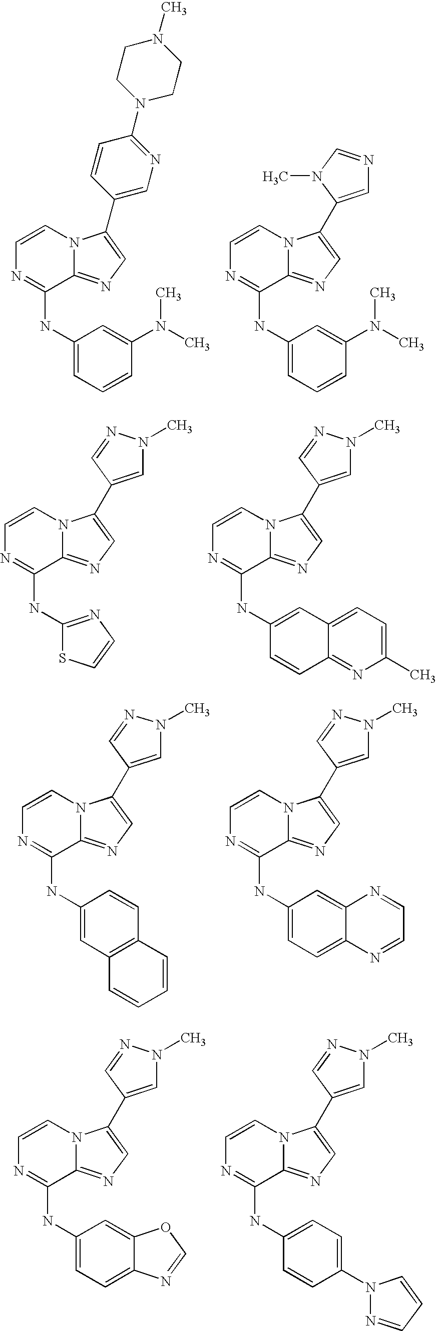 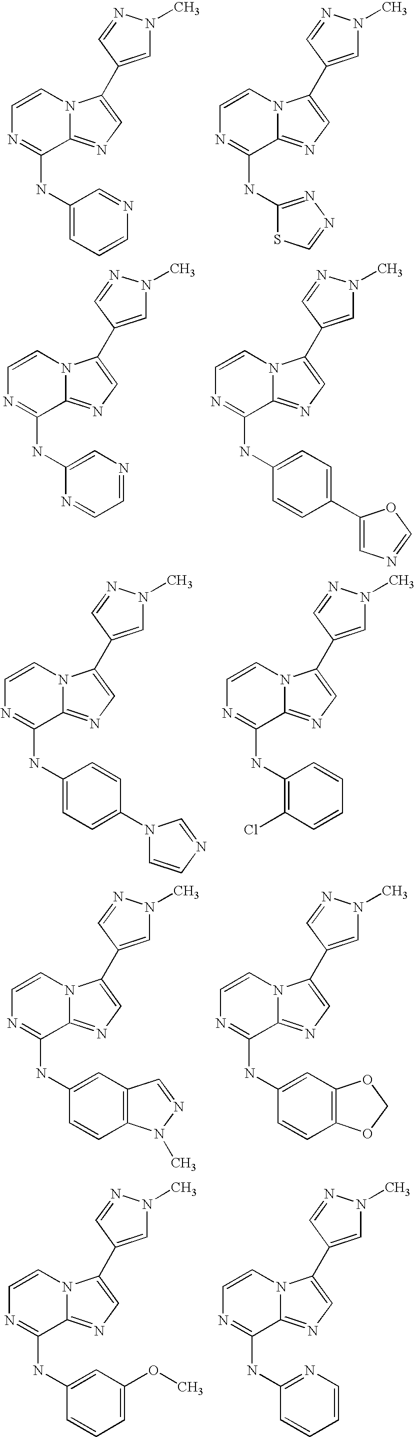 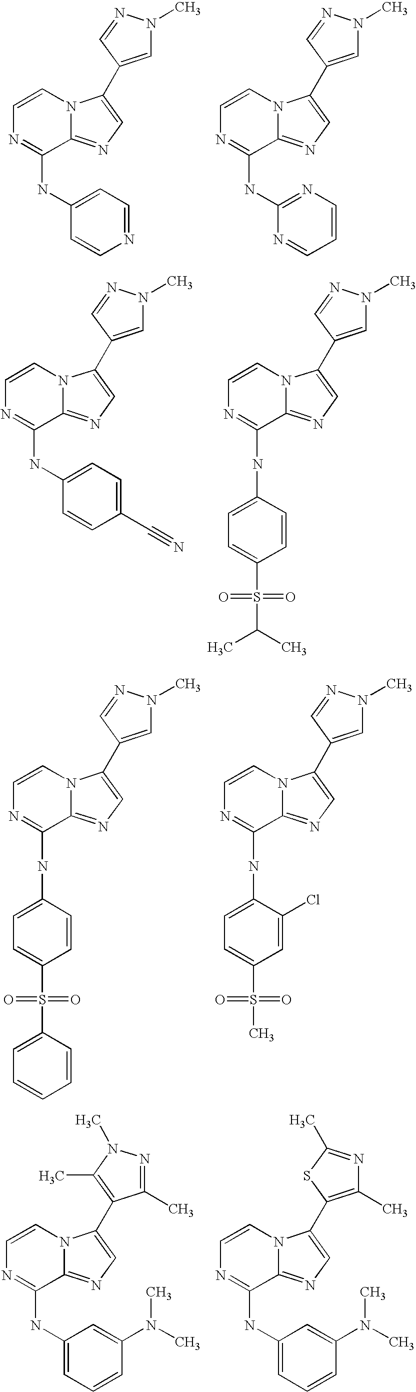 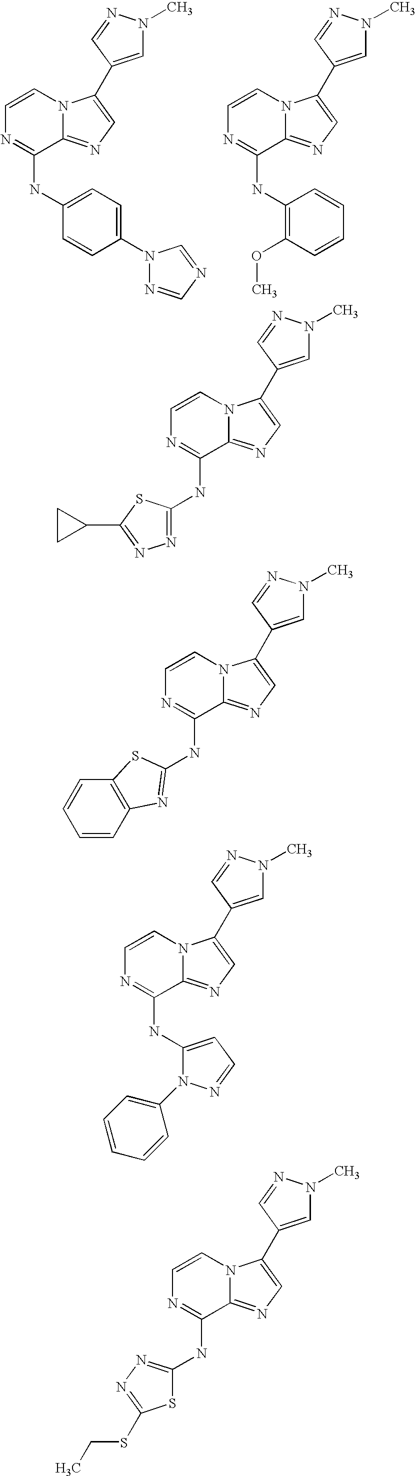 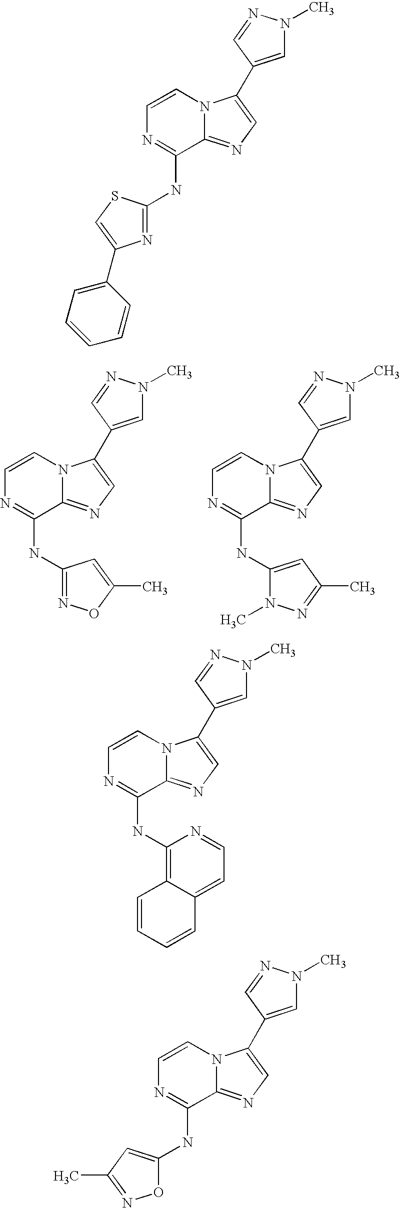 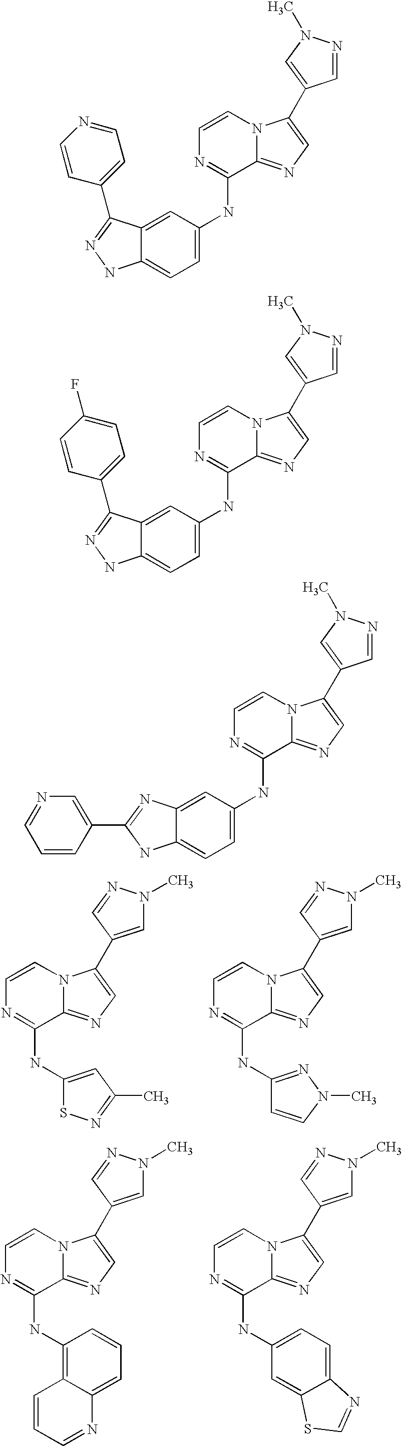 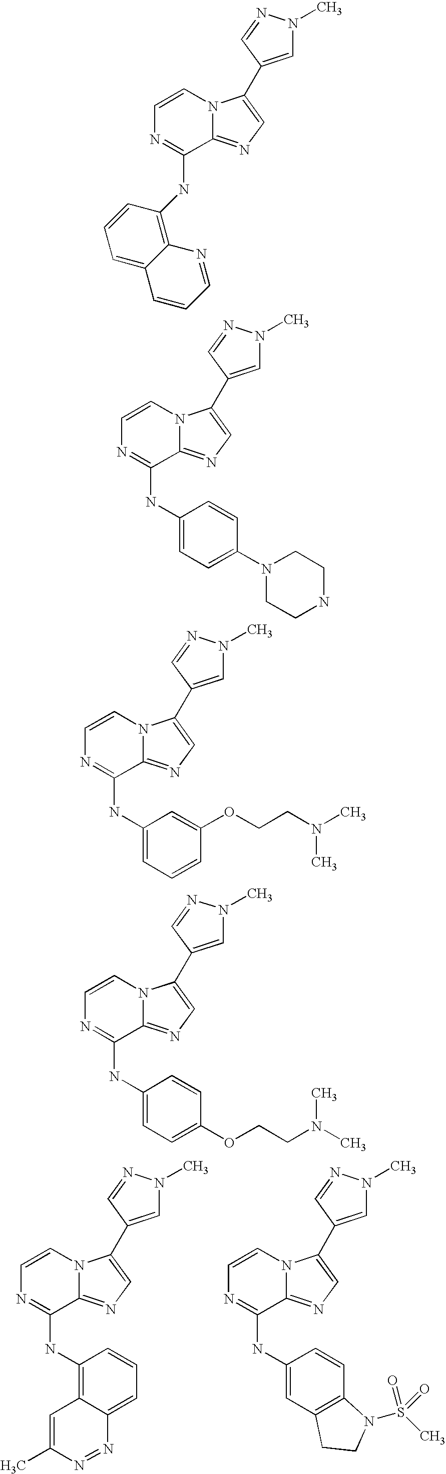 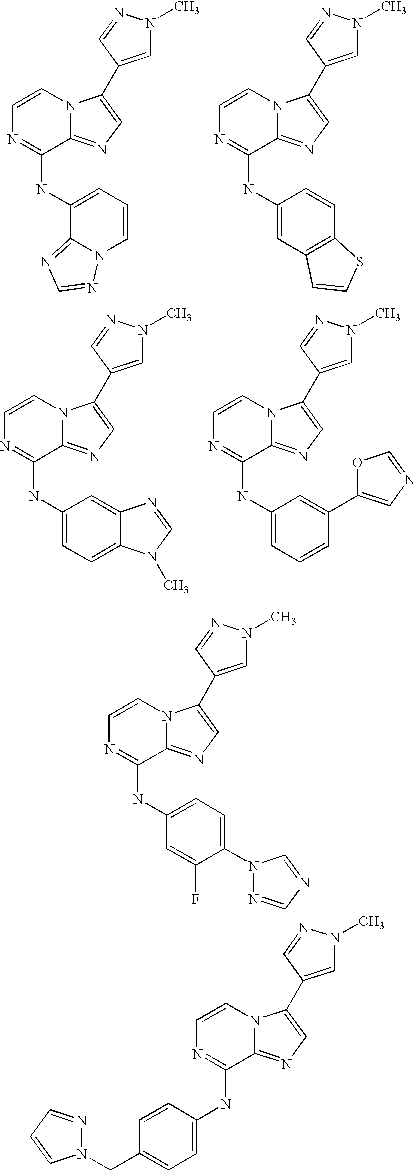 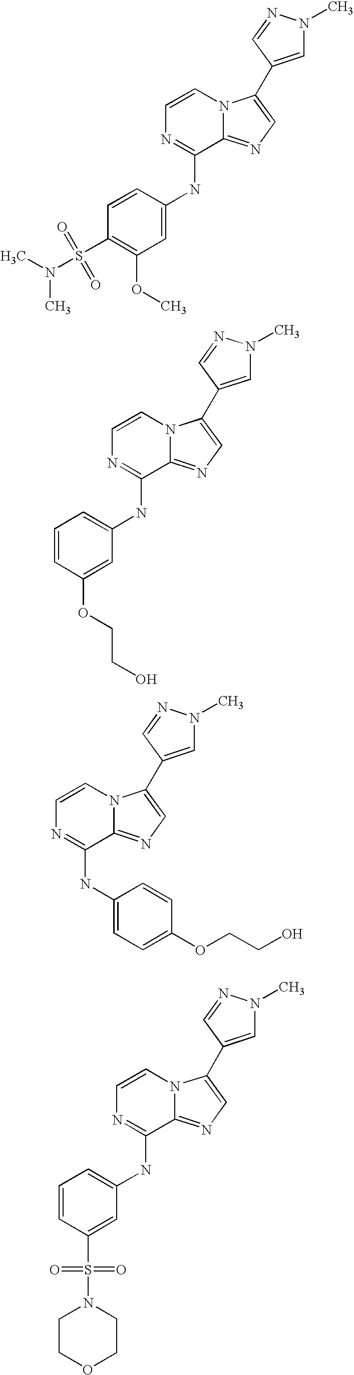 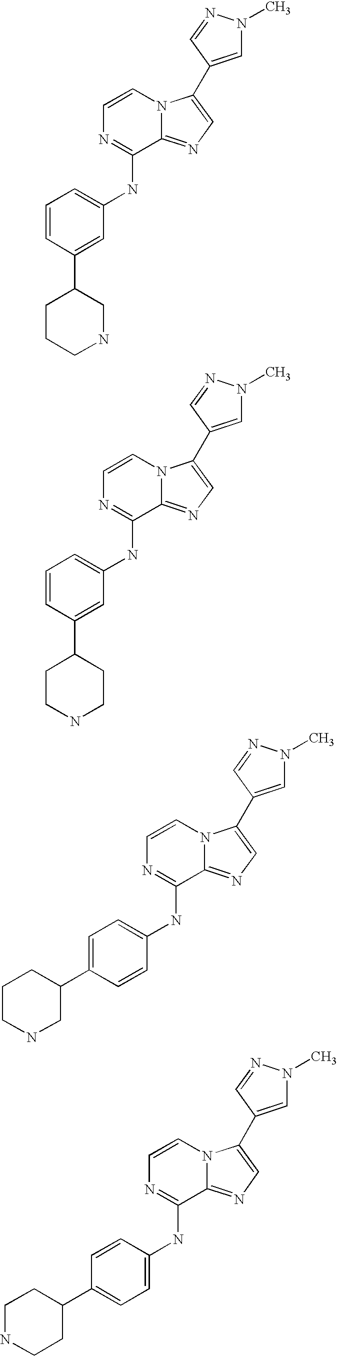 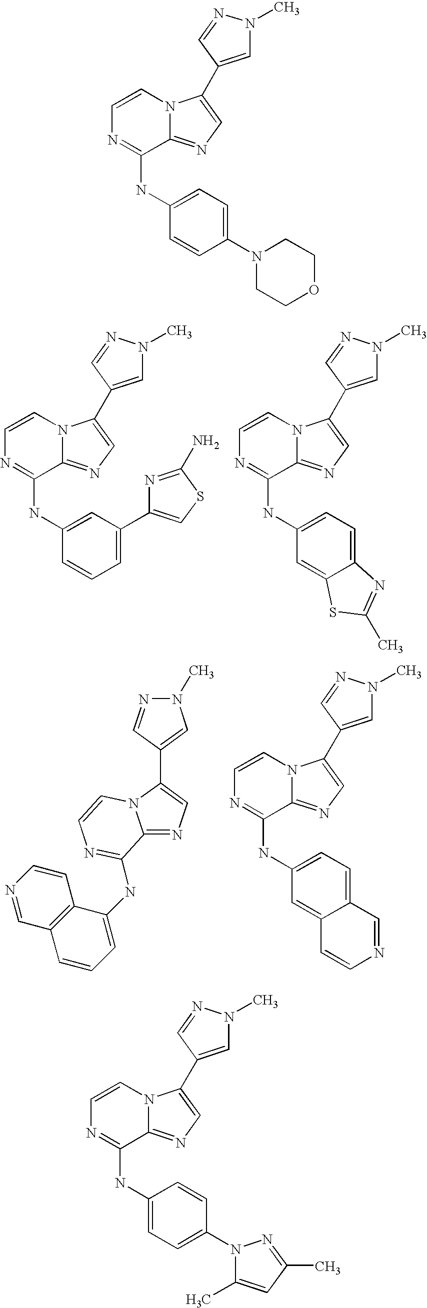 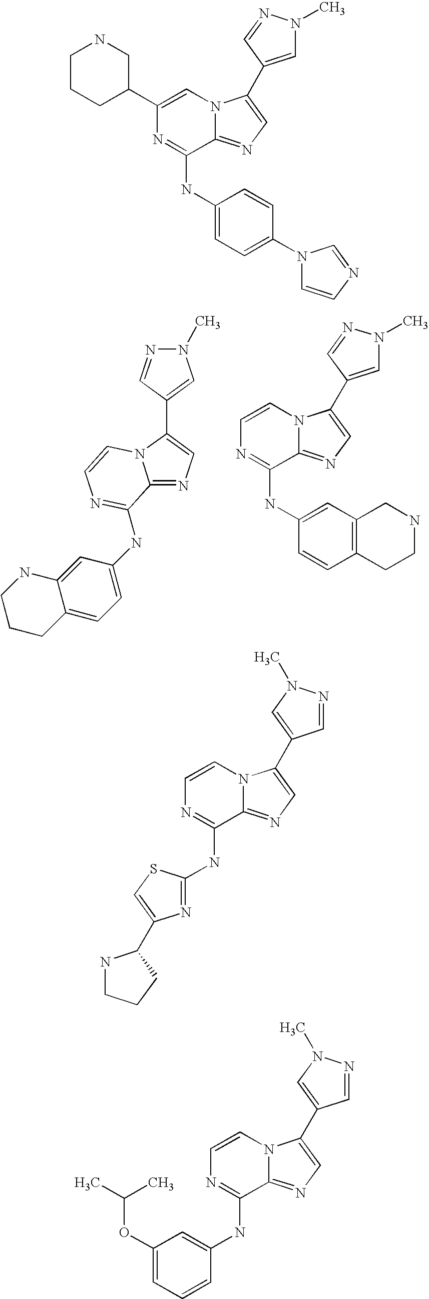 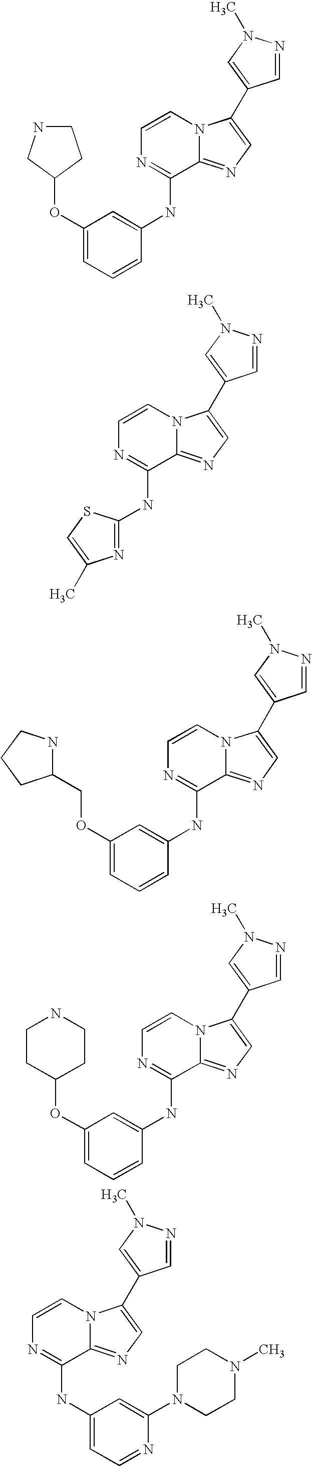 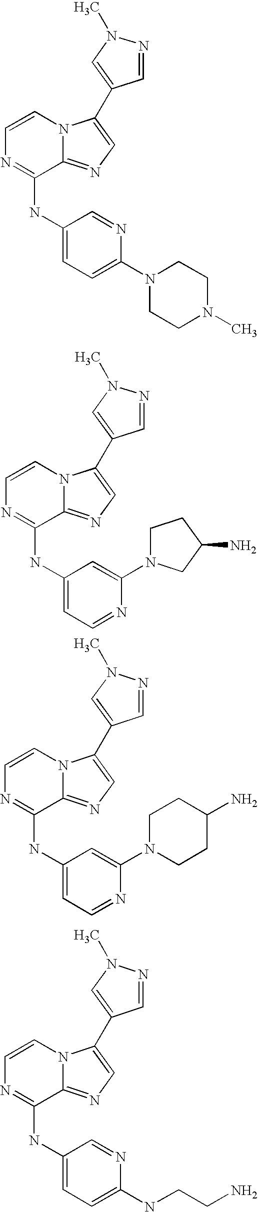 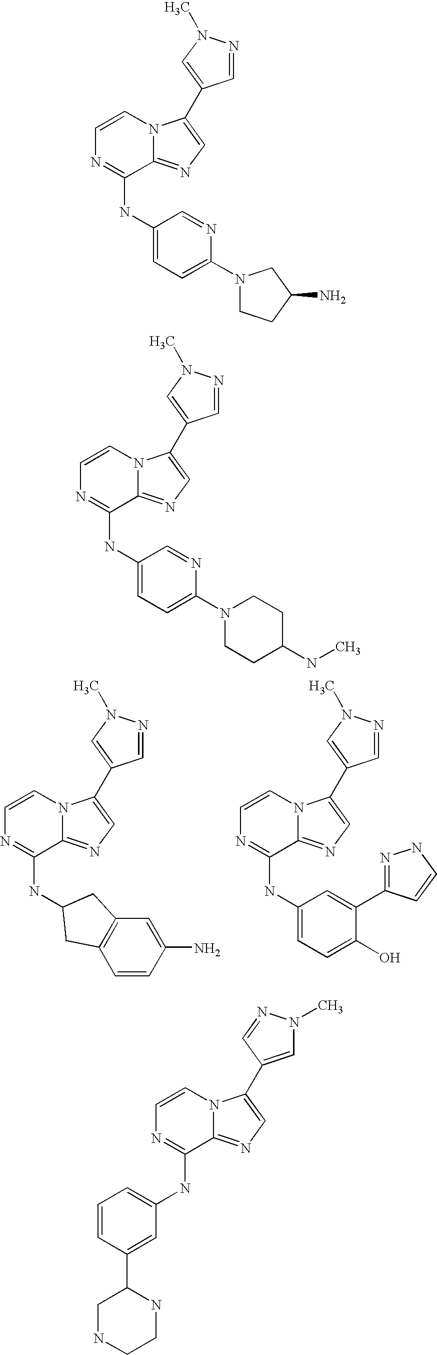 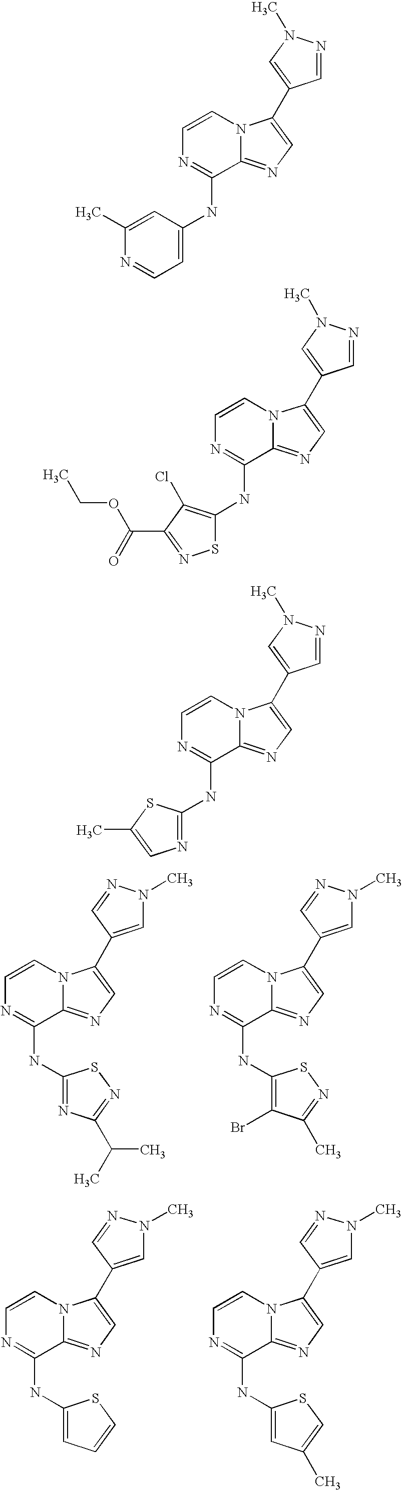 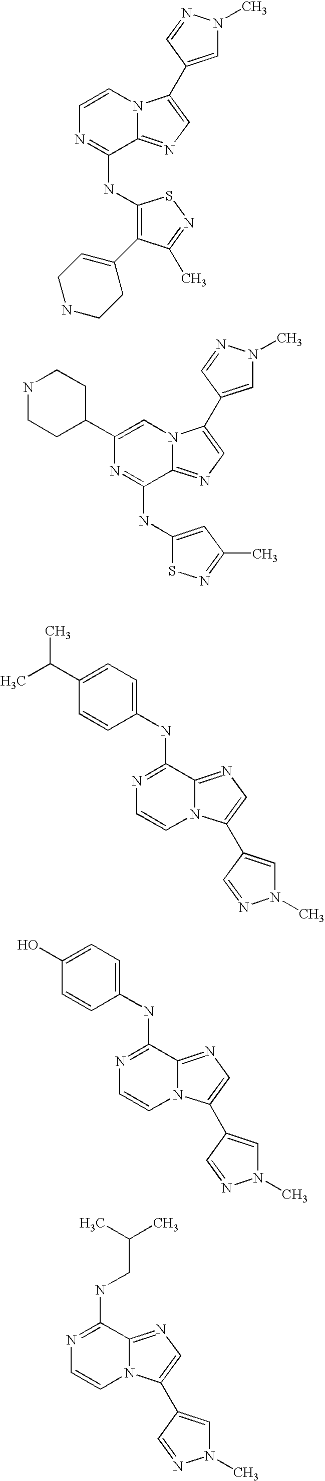 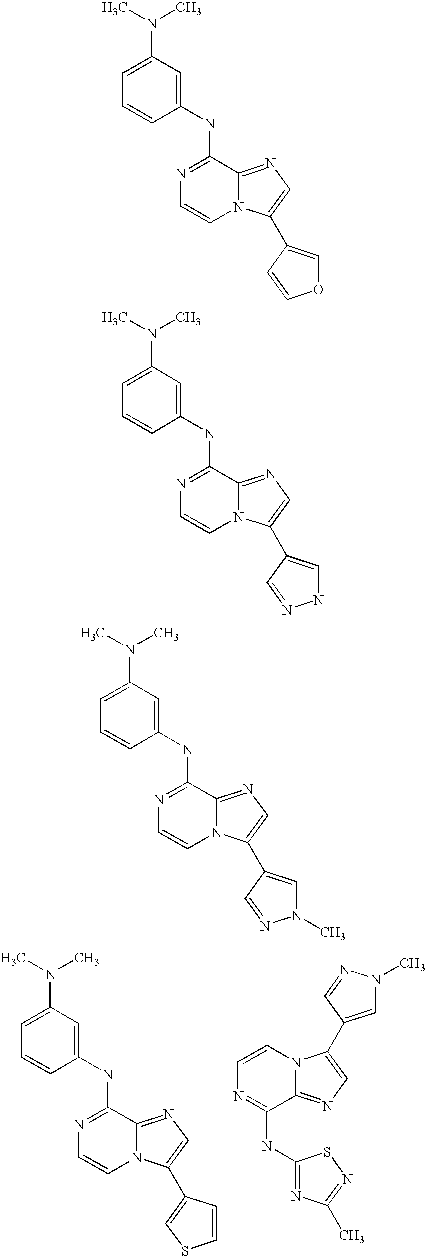 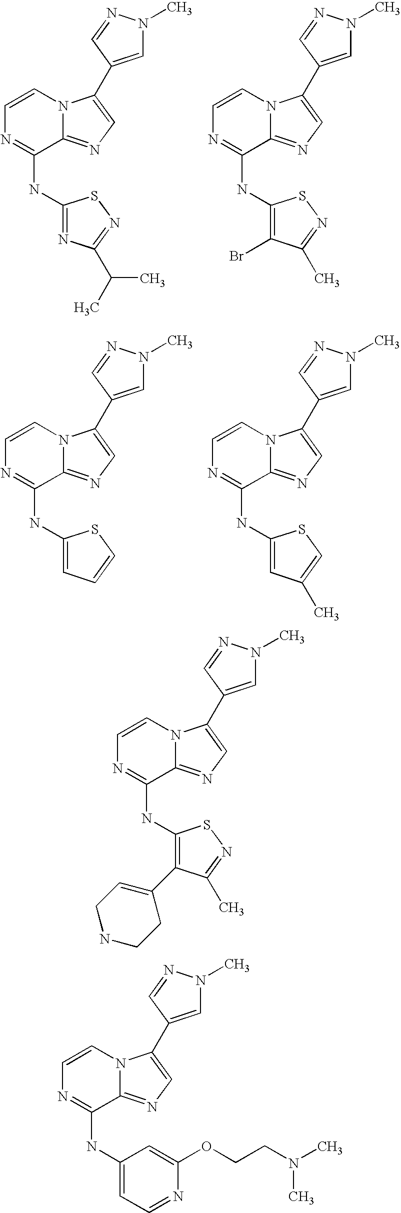 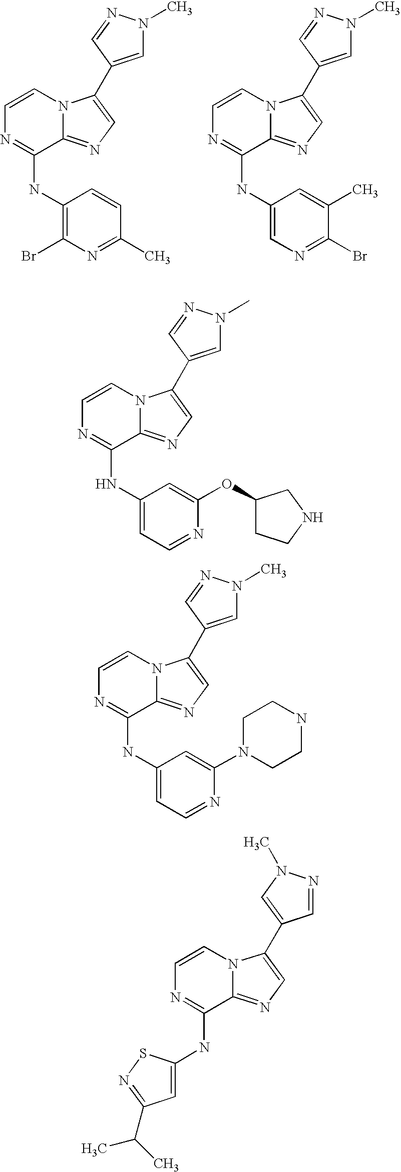 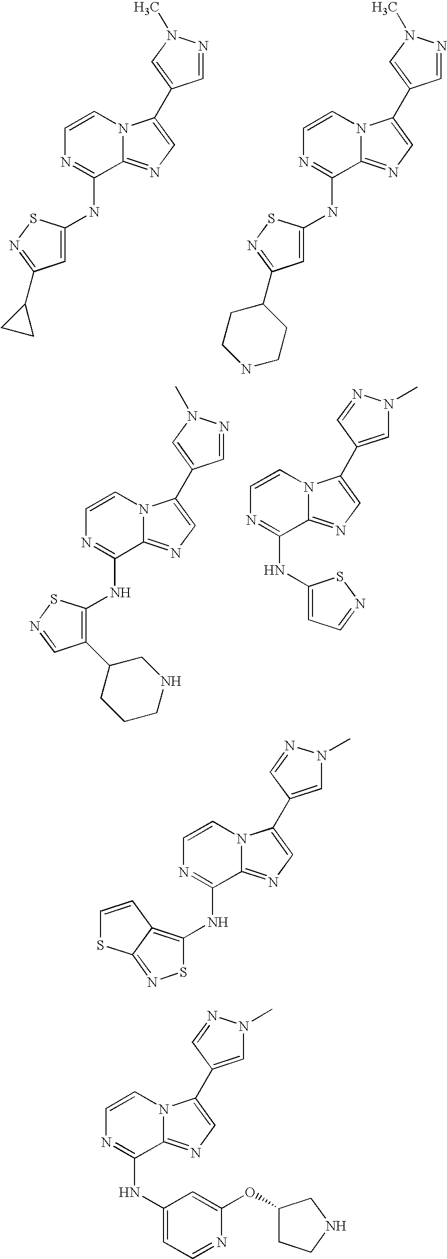 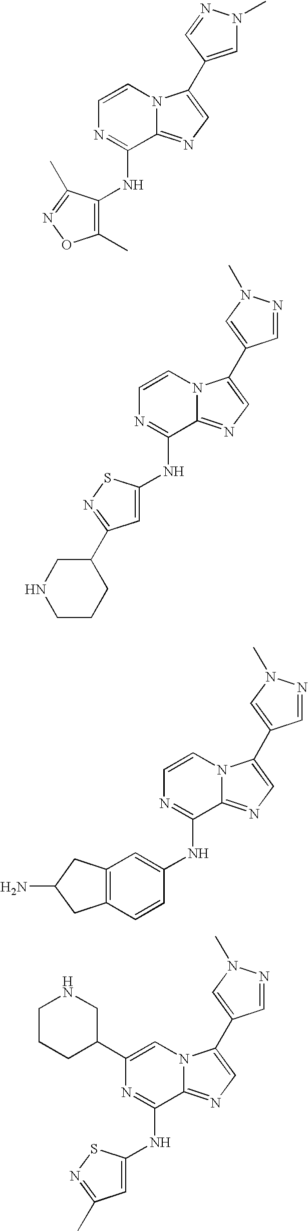 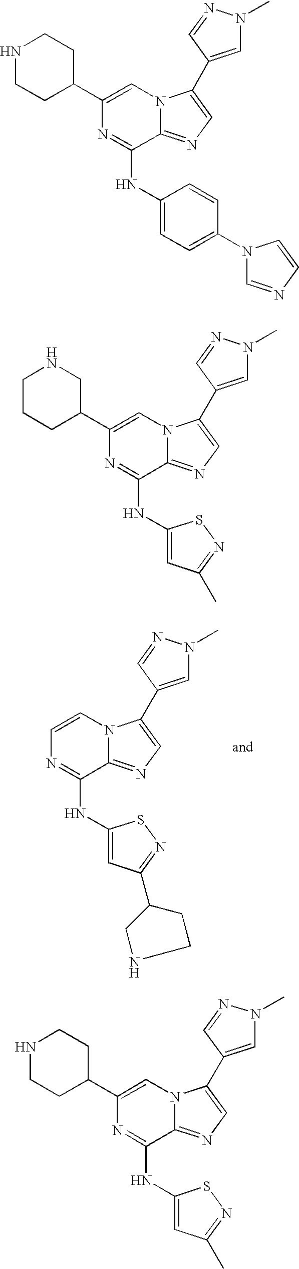
or a pharmaceutically acceptable salt, solvate, ester or prodrug thereof;
h. A compound of the formula: 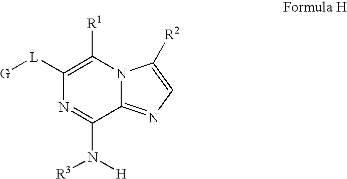
or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷, 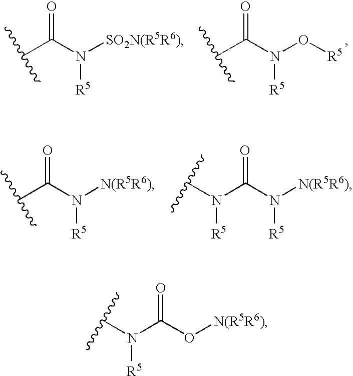
alkyl substituted with 1-6 R⁹ groups which groups can be the same or different with each R⁹ being independently selected, 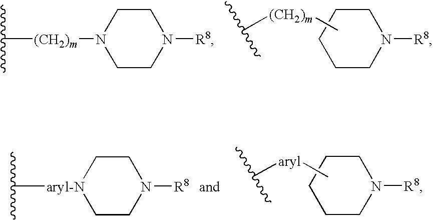
wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,
—N(R⁵)C(O)R⁷ and —N(R⁵)C(O)NR⁵R⁶;
R³ is selected from the group consisting of H, alkyl, aryl, heteroaryl, heterocyclyl, aralkyl, heteroarylalkyl, cycloalkylalkyl, heterocyclylalkyl, 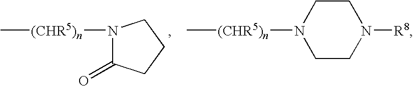
—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₘ—NR⁸, and 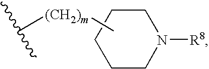
wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4; and
p is 0-3;
i. A compound of Formula I: 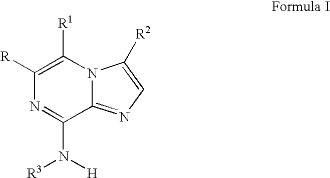
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
X is selected from the group consisting of, —NR⁵R⁶, —OR⁵, —SO—R⁵ and —SR⁵, R⁷ and R⁸ are each independently hydrogen or alkyl;
R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl, alkylN(alkyl)₂, alkylNH(alkyl), alkylN(alkenyl)2, -alkylN(alkoxyl)2, -alkyl-SH and hydroxyalkyl, wherein each of said aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkylSH, alkoxyl, —S-alkyl, hydroxyalkyl, and aminoalkyl;
R⁶ is selected from the group consisting of hydrogen, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl, wherein each of said aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cycloalkenylalkyl, cycloalkylalkyl, heteroarylalkyl, heterocyclenylalkyl, heterocycloalkylalkyl, heteroaryl, aryl, cycloalkenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, and heterocyclenyl;
j. A compound having the formula: 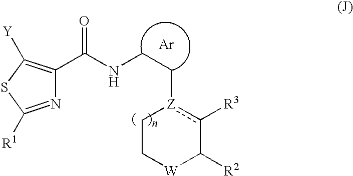
or a pharmaceutically acceptable salt, solvate, ester, prodrug or stereoisomer thereof, wherein the dashed lines indicate an optional and additional bond, and wherein:
R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;
R² is —H, -alkyl, —NH₂ or —CH₂NH₂;
R³ is —H, -alkyl, —NH₂ or —CH₂NH₂;
each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;
R⁵ is —H, -alkyl, -aryl, -heteroaryl, —NHOH;
R⁶ is —H, -alkyl or —CF₃;
R⁷ is —H, —OH, —C₁-C₆ alkyl, —O—(C₁-C₆ alkyl), or —CF₃;
R⁸ is —H, alkyl, aryl, heterocyclyl, heteroaryl or cycloalkyl;
Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen
W is —NH— or —C(R⁴)₂—, wherein both R⁴ groups and the carbon atom to which they are attached can combine to form a five to seven membered heterocyclyl or heteroaryl group;
Y is —H, -halo, -alkyl or —CN;
Z is —CR⁷— or —N—, when the optional additional bond is absent, and —C— when the optional additional bond is present;
n is an integer ranging from 0 to 2; and
(k) A compound of Formula K: 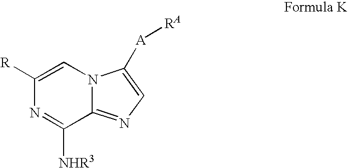
or a pharmaceutically acceptable salt, solvate, ester, prodrug or stereoisomer thereof, wherein:
R is H, halo or alkyl;
R³ is heteroaryl-X, wherein X is heterocyclylalkyl- wherein said heterocyclyl can be unsubstituted or optionally substituted with 1-4 alkyl moieties;
A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;
R^{A} is —(CH₂)₁₋₄-heteroaryl, 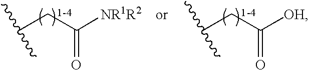
wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
R¹ and R² together with the N to which each is attached, form a heterocyclic group selected from the group consisting of 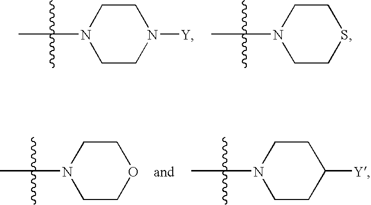
wherein
Y is alkoxyalkyl, hydroxyalkyl, dialkylaminoalkyl or alkyl, further wherein Y′ is hydroxyl.

11. A method of inducing polyploidy in cancer cells comprising the steps of first administering to a cancer cell in vivo at least one anti-mitotic agent selected from the group consisting of a taxane, paclitaxel, docetaxel, a Cenp-E Inhibitor, Abraxane, Epothilone, Monastrol, KSP inhibitors including Ispinesib SB-715992 (Cytokinetics), and compounds represented by Formulas A-D shown in paragraphs a-d below:
a. A compound represented by the structural Formula A: 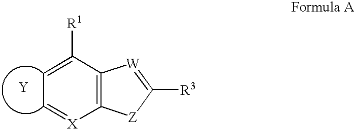
or a pharmaceutically acceptable salt, solvate or ester thereof, wherein:
ring Y is a 5- to 6-membered aryl or a 5- or 6-membered heteroaryl fused as shown in Formula A, wherein in said aryl and heteroaryl each substitutable ring carbon is independently substituted with R² and each substitutable ring nitrogen is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹;
each R² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R³ is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴R⁵, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
each R⁴ and R⁵ is independently selected from the group consisting of H, alkyl, cycloalkyl, cycloalkylalkyl, heterocyclyl, heterocyclylalkyl, aryl, aralkyl, heteroaryl, heteroaralkyl, —C(O)R⁷, and —C(O)OR⁷, wherein each of said alkyl, cycloalkyl, cycloalkylalkyl, heterocyclyl, heterocyclylalkyl, aryl, aralkyl, heteroaryl, or heteroaralkyl is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)R¹⁰, —C(O)OR'°, —C(O)NR'° R¹¹, —CF₃, —OCF₃, —CF₂CF₃, —C(═NOH)R¹⁰, —N(R¹⁰)C(O)R¹¹, —C(═NR¹⁰)NR¹⁰R¹¹, and —NR¹⁰C(O)OR¹¹, wherein each of said alkyl, cycloalkyl, heterocyclyl, aryl, and heteroaryl is optionally independently substituted with 1-3 moieties selected from the group consisting of halo, alkyl, cycloalkyl, heterocyclyl, aryl, heteroaryl, —NO₂, —OR¹⁰, —(C₁-C₆ alkyl)-OR¹⁰, —CN, —NR¹⁰R¹¹, —C(O)OR¹⁰, —C(O)NR¹⁰R¹¹, —CF³, —OCF₃, —NR¹⁰)C(O)OR¹¹, and —NR¹⁰C(O)R⁴⁰;
or two R⁸ groups, when attached to the same carbon atom, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H or alkyl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties; and
R⁴⁰ is selected from the group consisting of cycloalkyl, heterocyclyl, aryl and heteroaryl, wherein each of said cycloalkyl, heterocyclyl, aryl and heteroaryl are optionally independently substituted with 1-3 moieties independently selected from the group consisting of —CN, —OH, halo, alkyl, haloalkyl, alkoxy, and —NR¹⁰R¹¹;
b. A compound represented by the structural Formula B: 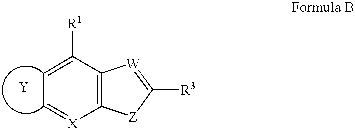
or a pharmaceutically acceptable salt, solvate or ester thereof, wherein:
ring Y is a 5- to 7-membered ring selected from the group consisting of cycloalkyl, cycloalkenyl, heterocyclyl or heterocyclenyl fused as shown in Formula B, wherein in each of said 5- to 7-membered ring, each substitutable ring carbon is independently substituted with 1-2 R² moieties and each substitutable ring heteroatom is independently substituted with R⁶;
W is N or C(R¹²);
X is N or N-oxide;
Z is S, S(═O) or S(═O)₂;
R¹ is H, alkyl, alkoxy, hydroxy, halo, —CN, —S(O)ₘ-alkyl, —C(O)NR⁹R¹⁰, —(CR⁹R¹⁰)₁₋₆OH, or —NR⁴(CR⁹R¹⁰)₁₋₂OR⁹; wherein m is 0 to 2;
each R² is independently selected from the group consisting of H, halo, alkyl, cycloalkyl, alkylsilyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, and —C(S)NR⁷(CH₂)₁₋₁₀OR⁷, wherein each of said alkyl, cycloalkyl, cycloalkenyl, heterocyclyl, heterocyclenyl, aryl, and heteroaryl is independently optionally substituted with 1-5 R⁹ moieties;
or two R²s on the same carbon atom are optionally taken together with the carbon atom to which they are attached to form a C═O, a C═S or an ethylenedioxy group;
R³ is independently selected from the group consisting of H, halo, alkyl, cycloalkyl, cycloalkenyl, heterocyclyl, heterocyclenyl, aryl, heteroaryl, —(CR¹⁰R¹¹)₀₋₆—OR⁷, —C(O)R⁴, —C(S)R⁴, —C(O)OR⁷, —C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —C(═NR⁷)NR⁴R⁵, —C(O)N(R⁷)—(CR⁴⁰R⁴¹)₁₋₅—C(═NR⁷)NR⁴R⁵, —C(O)N(R⁷)(CR⁴⁰R⁴¹)₁₋₅—NR⁴R⁵, —C(O)N(R⁷)(CR⁴⁰R⁴¹)₁₋₅—C(O)—NR⁴R⁵,
—C(O)N(R⁷)(CR⁴⁰R⁴¹)₁₋₅—OR⁷, —C(S)NR⁷(CH₂)₁₋₅NR⁴R⁵, and
—C(S)NR⁷(CH₂)₁₋₅OR⁷, wherein each of said alkyl, cycloalkyl, cycloalkenyl, heterocyclyl, heterocyclenyl, aryl, and heteroaryl is independently optionally substituted with 1-5 R⁹ moieties;
each of R⁴ and R⁵ is independently selected from the group consisting of H, alkyl, cycloalkyl, cycloalkenyl, heterocyclyl, heterocyclenyl, aryl, heteroaryl, —OR⁷, —C(O)R⁷, and —C(O)OR⁷, wherein each of said alkyl, cycloalkyl, cycloalkenyl, heterocyclyl, heterocyclenyl, aryl, and heteroaryl, is optionally substituted with 1-4 R⁸ moieties;
or R⁴ and R⁵, when attached to the same nitrogen atom, are optionally taken together with the nitrogen atom to which they are attached to form a 3-6 membered heterocyclic ring having 0-2 additional heteroatoms selected from N, O or S;
each R⁶ is independently selected from the group consisting of H, alkyl, aryl, aralkyl, cycloalkyl, cycloalkylalkyl, heterocyclyl, heterocyclylalkyl, heteroaryl, heteroaralkyl, —(CH₂)₁₋₆CF₃, —C(O)R⁷, —C(O)OR⁷ and —SO₂R⁷;
each R⁷ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁷ except H is optionally substituted with 1-4 R⁸ moieties;
each R⁸ is independently selected from the group consisting of halo, alkyl, cycloalkyl, cycloalkenyl, heterocyclyl, heterocyclenyl, aryl, heteroaryl, —NO₂, -Ore, —(C₁-C₆ alkyl)-OR¹⁰, —CN, —NR¹⁰R¹¹, —C(O)R¹⁰, —C(O)OR¹⁰, —C(O)NR¹⁰R¹¹, —CF₃, —CF₂CF₃, —C(═NOH)R¹⁰, —N(R¹⁰)C(O)R¹¹, —C(═NR¹⁰)NR¹⁰R¹¹, and —NR¹⁰)C(O)OR¹¹; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered carbocyclic or heterocyclic ring;
or two R⁸ groups, when attached to the same carbon, are optionally taken together with the carbon atom to which they are attached to form a C═O or a C═S group;
each R⁹ is independently selected from the group consisting of H, alkyl, alkoxy, OH, CN, halo, —(CR¹⁰R¹¹)₀₋₄NR⁴R⁵, haloalkyl, hydroxyalkyl, alkoxyalkyl, —C(O)NR⁴R⁵, —C(O)OR⁷, —OC(O)NR⁴R⁵, —NR⁴C(O)R⁵, and —NR⁴C(O)NR⁴R⁵;
each R¹⁰ is independently H or alkyl; or R⁹ and R¹⁰, when attached to the same nitrogen atom, are optionally taken together with the nitrogen atom to which they are attached to form a 3-6 membered heterocyclic ring having 0-2 additional heteroatoms selected from N, O or S;
each R¹¹ is independently H, alkyl, cycloalkyl, cycloalkenyl, aryl, heterocyclyl, heterocyclenyl, or heteroaryl; or R¹⁰ and R¹¹, when attached to the same nitrogen atom, are optionally taken together with the nitrogen atom to which they are attached to form a 3-6 membered heterocyclic ring having 0-2 additional heteroatoms selected from N, O or S; wherein each of said R¹¹ alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹² is independently selected from the group consisting of H, halo, alkyl, cycloalkyl, cycloalkylalkyl, heterocyclyl, heterocyclylalkyl, aryl, aralkyl, heteroaryl, heteroaralkyl, —(CR¹⁰R¹¹)₀₋₆—OR⁷, —C(O)R⁴, —C(S)R⁴, —C(O)OR⁷,
—C(S)OR⁷, —OC(O)R⁷, —OC(S)R⁷, —C(O)NR⁴R⁵, —C(S)NR⁴R⁵, —C(O)NR⁴OR⁷, —C(S)NR⁴OR⁷, —C(O)NR⁷NR⁴R⁵, —C(S)NR⁷NR⁴R⁵, —C(S)NR⁴OR⁷, —C(O)SR⁷, —NR⁴R⁵, —NR⁴C(O)R⁵, —NR⁴C(S)R⁵, —NR⁴C(O)OR⁷, —NR⁴C(S)OR⁷, —OC(O)NR⁴R⁵, —OC(S)NR⁴R⁵, —NR⁴C(O)NR⁴R⁵, —NR⁴C(S)NR⁴R⁵, —NR⁴C(O)NR⁴OR⁷, —NR⁴C(S)NR⁴OR⁷, —(CR¹⁰R¹¹)₀₋₆SR⁷, SO₂R⁷, —S(O)₁₋₂NR⁴R⁵, —N(R⁷)SO₂R⁷, —S(O)₁₋₂NR⁵OR⁷, —CN, —OCF₃, —SCF₃, —C(═NR⁷)NR⁴, —C(O)NR⁷(CH₂)₁₋₁₀NR⁴R⁵, —C(O)NR⁷(CH₂)₁₋₁₀OR⁷, —C(S)NR⁷(CH₂)₁₋₁₀NR⁴R⁵, —C(S)NR⁷(CH₂)₁₋₁₀OR⁷, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R⁹ moieties;
R⁴⁰ and R⁴¹ can be the same or different, each being independently selected from the group consisting of H, alkyl, aryl, heteroaryl, heterocyclyl, heterocyclenyl, cycloalkyl and cycloalkenyl;
each R⁴² is independently selected from the group consisting of halo, alkyl, cycloalkyl, heterocyclyl, aryl, heteroaryl, —NO₂, —OR¹⁰, —(C₁-C₆ alkyl)-OR¹⁰,
—CN, —NR¹⁰R¹¹, —C(O)R¹⁰, —C(O)OR¹⁰, —C(O)NR¹⁰R¹¹, —CF₃, —OCF₃,
—N(R¹⁰)C(O)R¹¹, and —NR¹⁰C(O)OR¹¹;
with the proviso that when W is C(R¹²), R¹² and R³ are optionally taken together, with the two ring carbon atoms to which they are attached to form a 6-membered ring selected from the group consisting of cycloalkenyl, aryl, heteroaryl, heterocyclyl and heterocyclenyl, wherein said 6-membered ring is optionally substituted with 1-3 moieties independently selected from oxo, thioxo, —OR¹¹, —NR¹⁰R¹¹, —C(O)R¹¹, —C(O)OR¹¹, —C(O)N(R¹⁰(R¹¹), or —N(R¹⁰)C(O)R¹¹;
c. 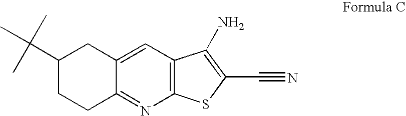
or a pharmaceutically acceptable salt, solvate, or ester thereof; and
d. A compound of Formula D 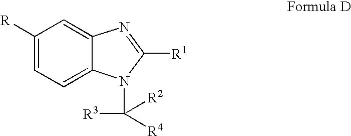
or a pharmaceutically acceptable salt, solvate, or ester thereof, wherein:
R is selected from the group consisting of H, alkyl, cyano, haloalkyl, halo, —SH, —S-alkyl, —S-haloalkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, cycloalkyl, aryl, heterocyclyl, heteroaryl, —NHC(═O)alkyl, —C(═O)NH₂, —C(═O)NH(alkyl), —C(═O)NH(cycloalkyl), —C(═O)N(alkyl)₂, —C(═O)OH, —C(═O)Oalkyl, —C(═O)heterocyclyl, —C(═O)NH(aryl), wherein when each of said cycloalkyl, aryl, heterocyclyl, heteroaryl, and the “heterocyclyl” and “aryl” portions of said R groups has two substituents on adjacent carbon atoms, said substituents, may optionally be taken together with the carbon atoms to which they are attached to form a five- to six-membered cycloalkyl, aryl, heterocyclyl, heterocyclenyl, or heteroaryl ring; wherein each of the aforementioned R alkyl, aryl, cycloalkyl, heterocyclyl, and heteroaryl, and the “alkyl”, “cycloalkyl”, “heterocyclyl”, and “aryl” portions of said R groups, optionally with said five- to six-membered aryl, heterocyclyl, heterocyclenyl, or heteroaryl ring, is optionally substituted with 1-3 substituents independently selected from the group consisting of alkyl, alkenyl, alkynyl, hydroxyl, cyano, halo, haloalkyl, haloalkoxy, —C(O)OH, —C(═O)Oalkyl, and —C(O)NH₂;
R¹ is selected from the group consisting of alkyl, heterocyclyl, —C(═O)aryl, —NH₂, —NH(alkyl), —NH(cycloalkyl), —N(alkyl)(cycloalkyl), —NH(heterocyclyl), —N(alkyl)(heterocyclyl), N(alkyl)₂, —NH(aryl), —N(alkyl)(aryl), —N(aryl)₂, —NH(heteroaryl), —N(alkyl)(heteroaryl), —NHC(═O)-alkyl, —N(alkyl)C(═O)-alkyl, —NHC(═O)Oalkyl, —N(alkyl)C(═O)O-alkyl, wherein each of the aforesaid alkyl, heterocyclyl, and the “alkyl”, “cycloalkyl”, “aryl”, and “heteroaryl” portions of said R¹ groups is optionally substituted with 1-3 substituents independently selected from the group consisting of halo, heterocyclyl, aryl, heteroaryl, haloalkyl, haloalkoxy, aryloxy, cyano, —SH, —S-alkyl, —S(═O)₂alkyl, —S(═O)₂OH, —S(═O)₂NH₂, —S(═O)₂NH(alkyl), S(═O)₂NH(cycloalkyl), —S(═O)₂N(alkyl)₂, —S(═O)₂heterocyclyl, —S(═O)₂heteroaryl, hydroxy, alkyl, alkenyl, alkynyl, —NH₂, —NH(alkyl), —N(alkyl)₂, alkoxy, —NHC(═O)alkyl, —C(═O)H, —C(═O)alkyl, —C(═O)aryl, —C(═O)heteroaryl, —C(═O)Oalkyl, —C(═O)NH₂, —C(O)NHalkyl, —C(═O)N(alkyl)₂; wherein when each of said heterocyclyl, aryl and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring;
R² and R³ independently are H or alkyl, or —C(R²)(R³)— is absent;
R⁴ is selected from the group consisting of alkyl, cycloalkyl, heterocyclyl, aryl and heteroaryl, wherein when each of said cycloalkyl, heterocyclyl, aryl, and heteroaryl has two substituents on adjacent carbon atoms, said substituents, may optionally be taken together with the carbon atoms to which they are attached to form a five to six-membered aryl, heterocyclyl, heterocyclenyl, or heteroaryl ring; wherein each of the aforementioned R⁴ alkyl, cycloalkyl, heterocyclyl, aryl and heteroaryl, optionally with said five to six-membered aryl, heterocyclyl, heterocyclenyl, or heteroaryl ring is optionally substituted with 1-3 substituents independently selected from the group consisting of cyano, halo, haloalkyl, alkyl, alkoxy, hydroxyl, haloalkoxy, cycloalkyl, heterocyclyl, aryl, heteroaryl, —S(═O)₂alkyl, —S(═O)₂NH₂, —S(═O)₂NH(alkyl), —S(═O)₂N(alkyl)₂, —S-alkyl, —S-haloalkyl, —C(═O)OH, —NH₂, —NH(alkyl), —N(alkyl)₂, and —C(═O)Oalkyl;
subsequently administering to said cancer cell at least one aurora kinase inhibitor, wherein said aurora kinase inhibitor is selected from the group consisting of a compound represented by Formulas E-K shown below in paragraphs e-k:
e. A compound represented by the structural Formula E: 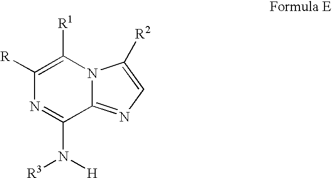
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl,
—N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂,
—NR⁵R⁶ (wherein the R⁵ and R⁶, together with the N of said
—NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl,
—CH₂OR⁵, —S(O)R⁵, —S(O₂)R⁵, —CN, —CHO, —SR⁵, —C(O)OR⁵, —C(O)R⁵, heteroaryl and heterocyclyl;
R³ is H, alkyl, cycloalkyl, heterocyclyl, aryl or heteroaryl, wherein:
said alkyl shown above for R³ can be unsubstituted or substituted with one or more moieties which can be the same or different each moiety being independently selected from the group consisting of —OR⁵, alkoxy, heteroaryl, and —NR⁵R⁶;
said aryl shown above for R³ is unsubstituted, or optionally substituted, or optionally fused, with halo, heteroaryl, heterocyclyl, cycloalkyl or heteroarylalkyl, wherein each of said heteroaryl, heterocyclyl, cycloalkyl and heteroarylalkyl can be unsubstituted or optionally independently substituted with one or more moieties which can be the same or different each moiety being independently selected from alkyl, —OR⁵, —N(R⁵R⁶) and
—S(O₂)R⁵; and
said heteroaryl shown above for R³ can be unsubstituted or optionally substituted, or optionally fused, with one or more moieties which can be the same or different with each moiety being independently selected from the group consisting of halo, amino, alkoxycarbonyl, —OR⁵, alkyl, —CHO, —NR⁵R⁶, —S(O₂)N(R⁵R⁶), —C(O)N(R⁵R⁶), —SR⁵, alkenyl, alkynyl, cycloalkyl, aryl, heteroaryl, heterocyclenyl, and heterocyclyl;
R⁵ is H, alkyl, aminoalkyl, aryl, heteroaryl, heterocyclyl or cycloalkyl; and
R⁶ is H, alkyl, aryl, arylalkyl, heteroaryl, heterocyclyl or cycloalkyl;
further wherein in any —NR⁵R⁶ in Formula I, said R⁵ and R⁶ can optionally be joined together with the N of said —NR⁵R⁶ to form a cyclic ring;
f. A compound represented by the structural formula: 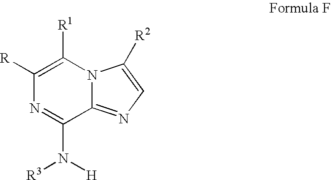
or a pharmaceutically acceptable salt, solvate, ester or prodrug thereof, wherein:
R is selected from the group consisting of H, halogen, aryl, heteroaryl, cycloalkyl, arylalkyl, heterocyclyl, heterocyclylalkyl, alkenyl, alkynyl,
—C(O)R⁷, 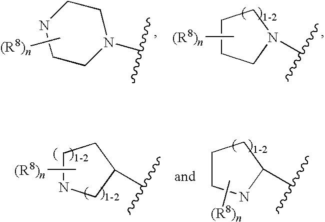
wherein each of said aryl, heteroaryl, cycloalkyl, arylalkyl, alkenyl, heterocyclyl and the heterocyclyl moieties whose structures are shown immediately above for R can be unsubstituted or optionally independently substituted with one or more moieties which can be the same or different, each moiety being independently selected from the group consisting of halogen, alkyl, cycloalkyl, CF₃, CN, —OCF₃,
—OR⁶, —C(O)R⁷, —NR⁵R⁶, —C(O₂)R⁶, —C(O)NR⁵R⁶, —(CHR⁵)ₙOR⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R¹ is H, halogen or alkyl;
R² is alkyl;
R³ is selected from the group consisting of H, aryl, heteroaryl, heterocyclyl, —(CHR⁵)ₙ-aryl, —(CHR⁵)ₙ-heteroaryl, —(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —(CH₂)ₘ—NR⁸, —(CHR⁵)ₙ—CH(aryl)₂, 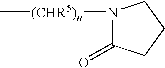
and 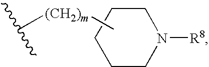
wherein each of said aryl, heteroaryl and heterocyclyl can be substituted or optionally substituted with one or more moieties which can be the same or different, each moiety being independently selected from the group consisting of halogen, alkyl, aryl, cycloalkyl, CF₃, CN, —OCF₃, —OR⁵, —NR⁵R⁶, —C(O₂)R⁵,
—C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H or alkyl;
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and
—N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷,
—S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶,
—N(R⁵)S(O₂)R⁷, —N(R⁵)C(O)R⁷ and —N(R⁵)C(O)NR⁵R⁶;
m is 0 to 4;
n is 1-4; and
p is 0-3;
g. A compound selected from the compounds of the formulas: 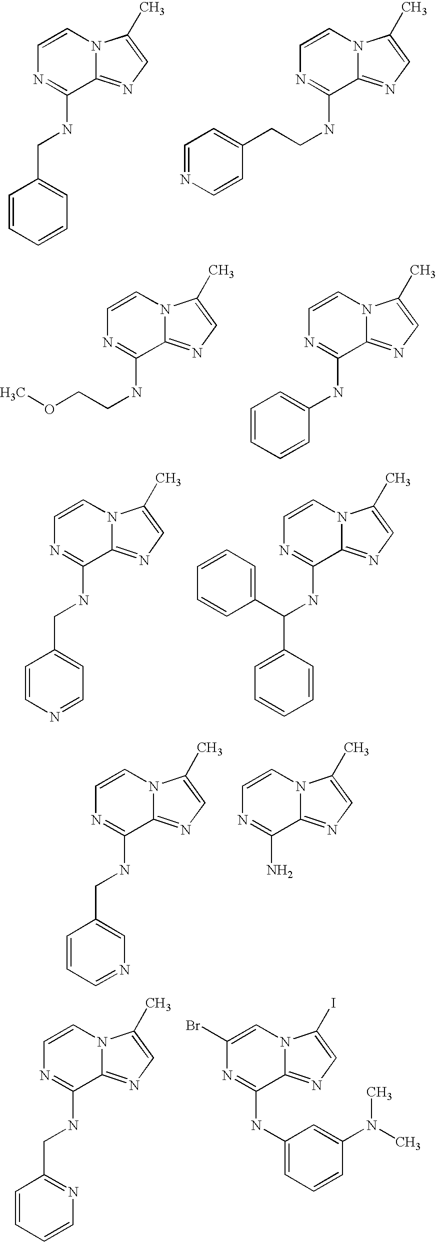 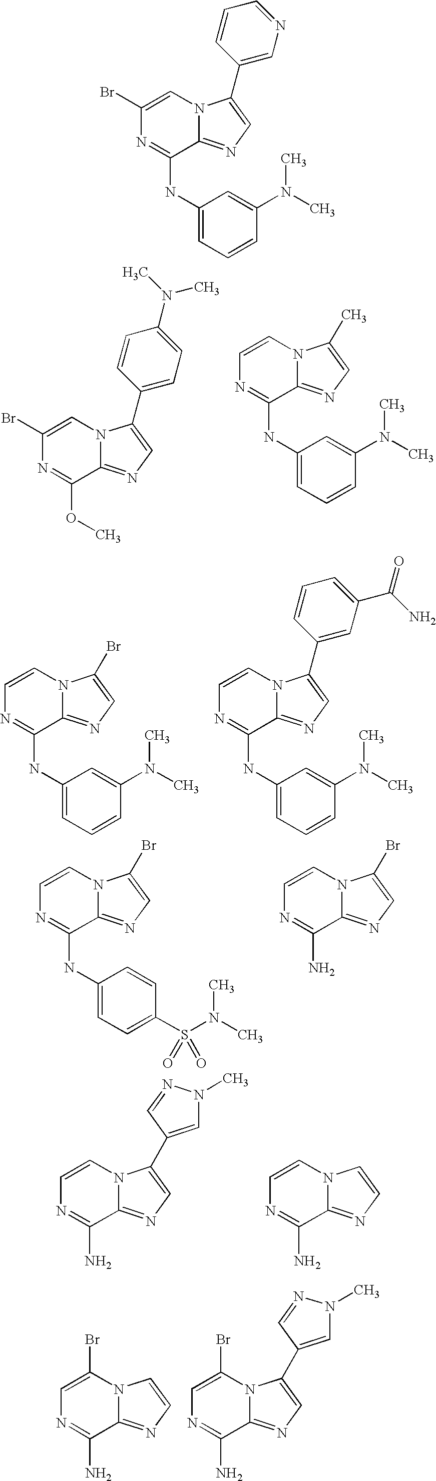 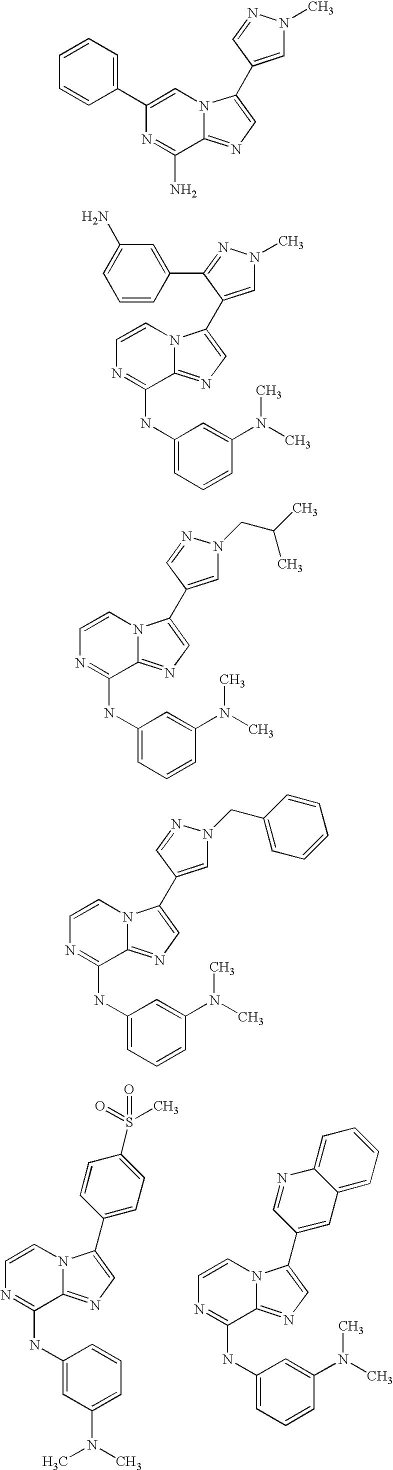 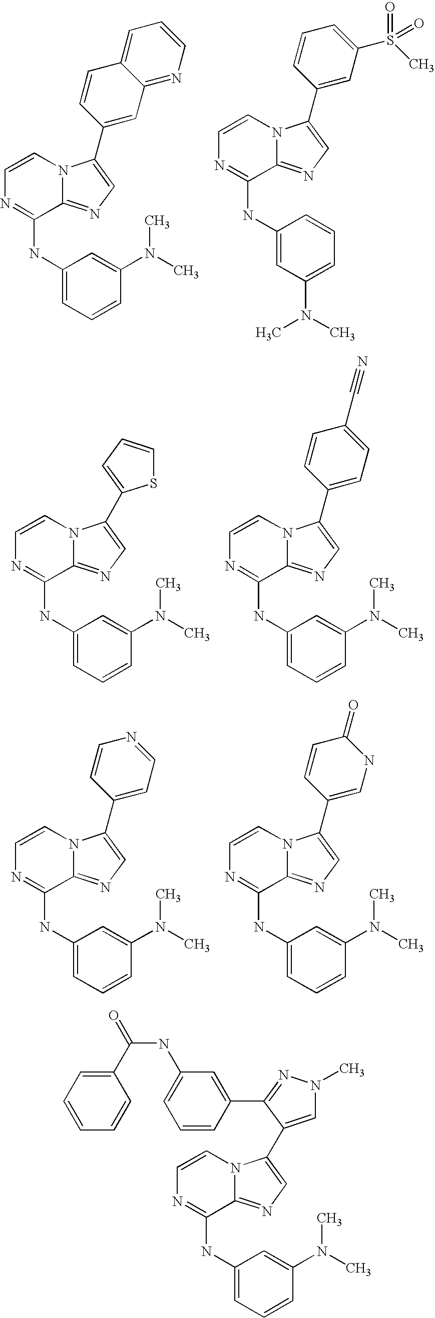 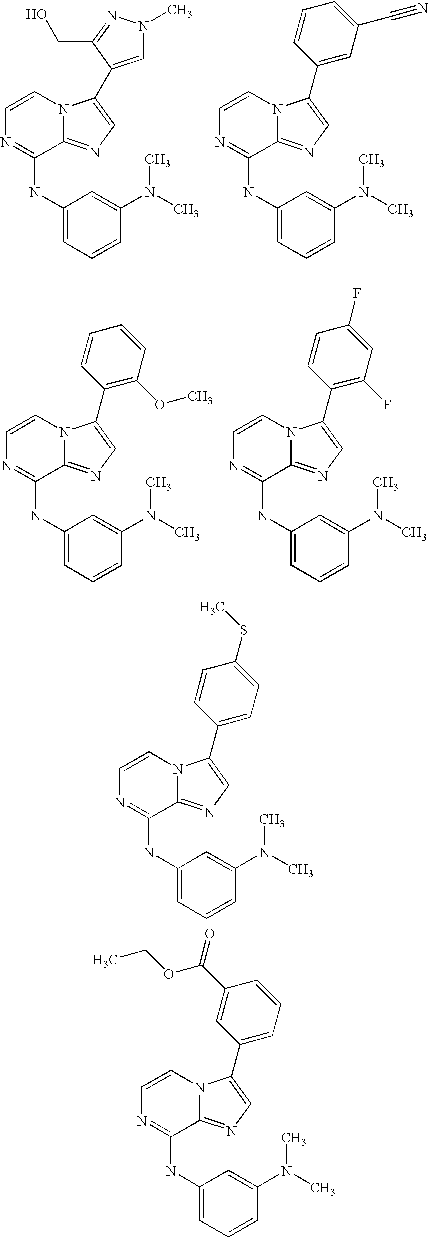 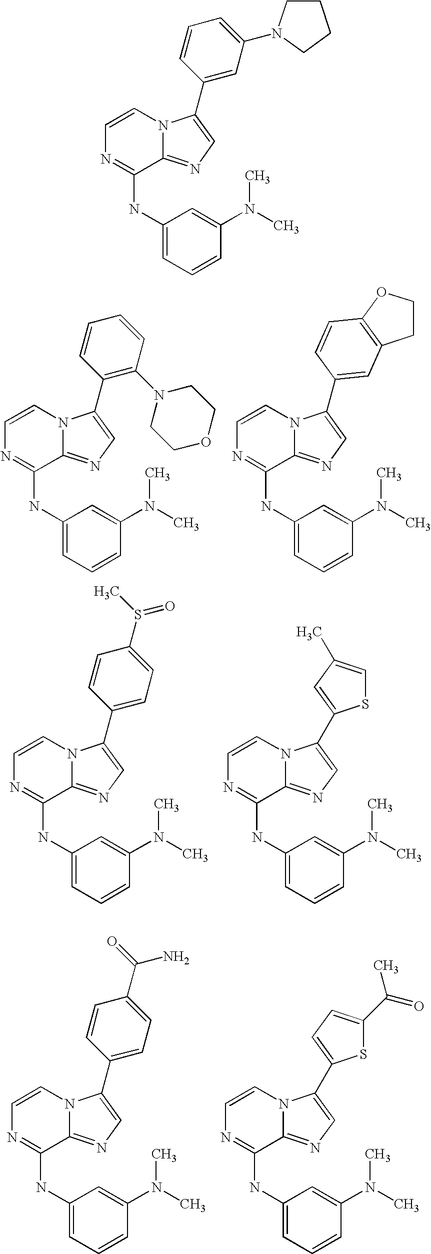 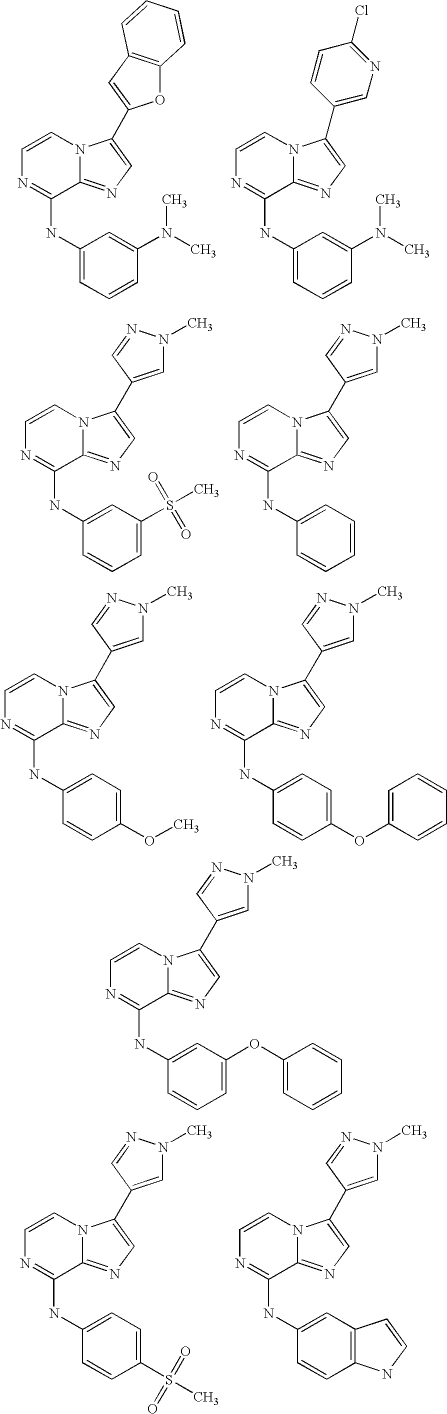 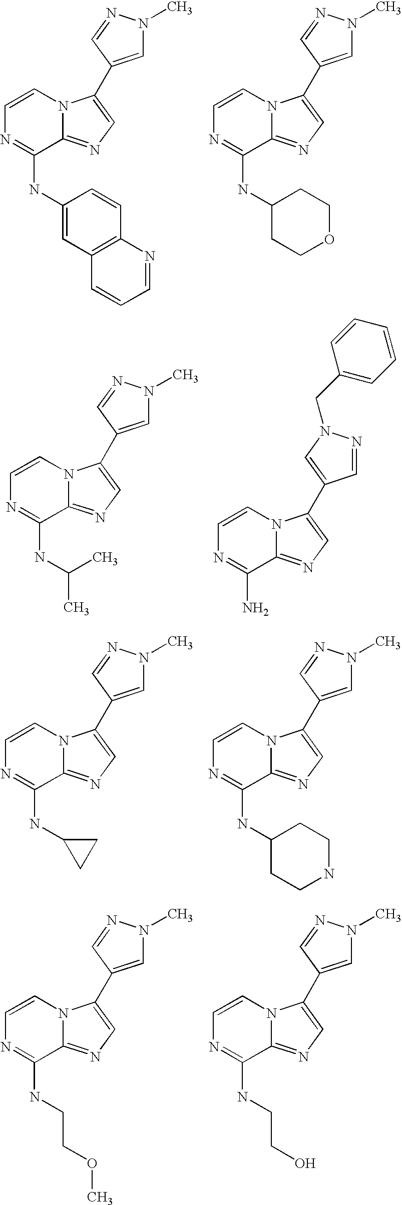 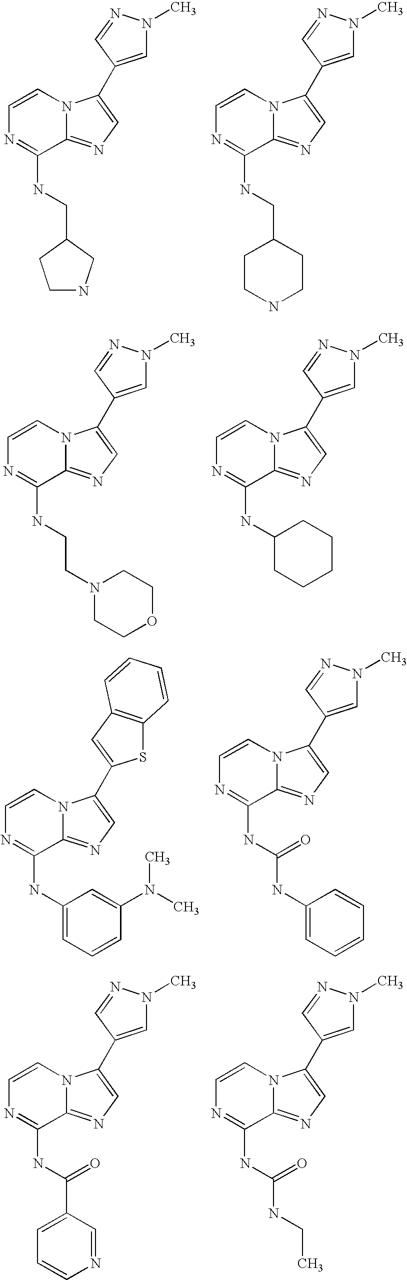 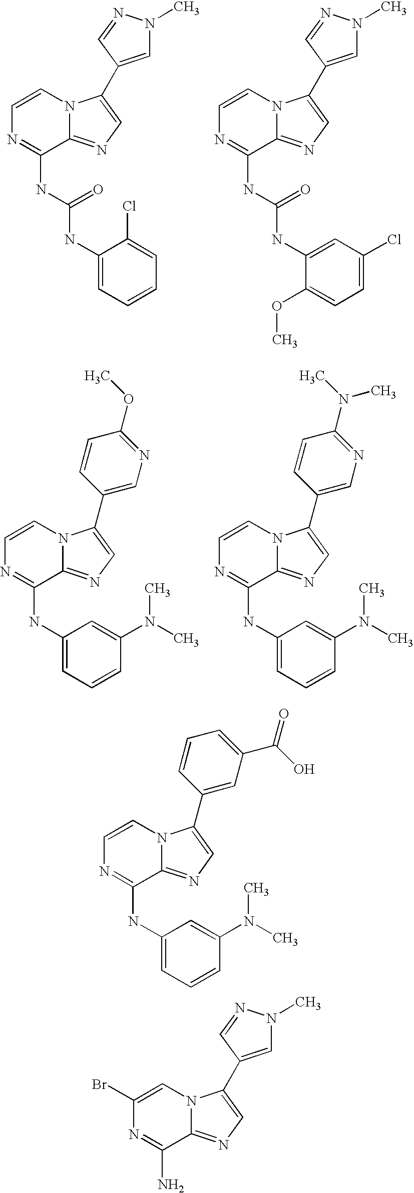 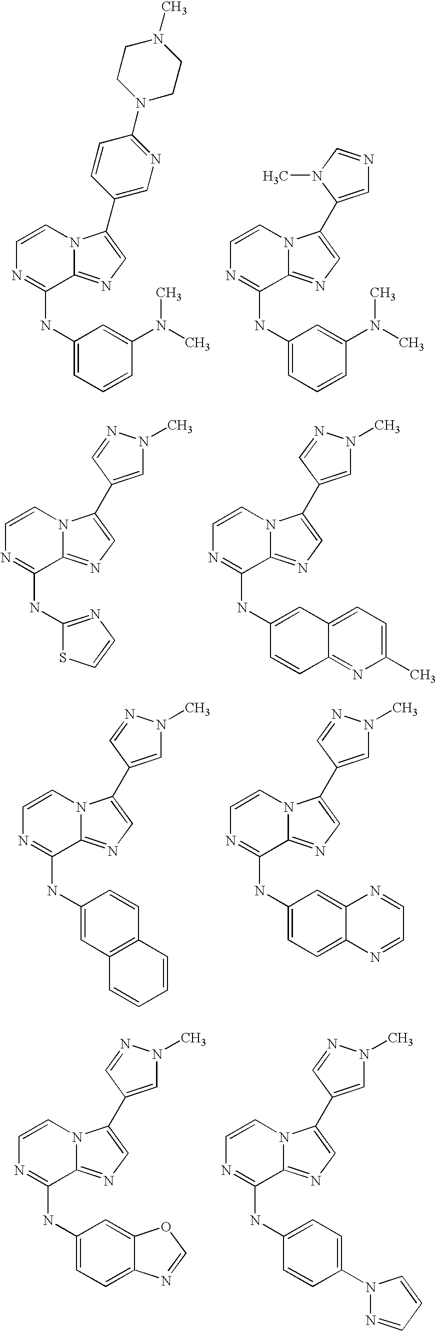 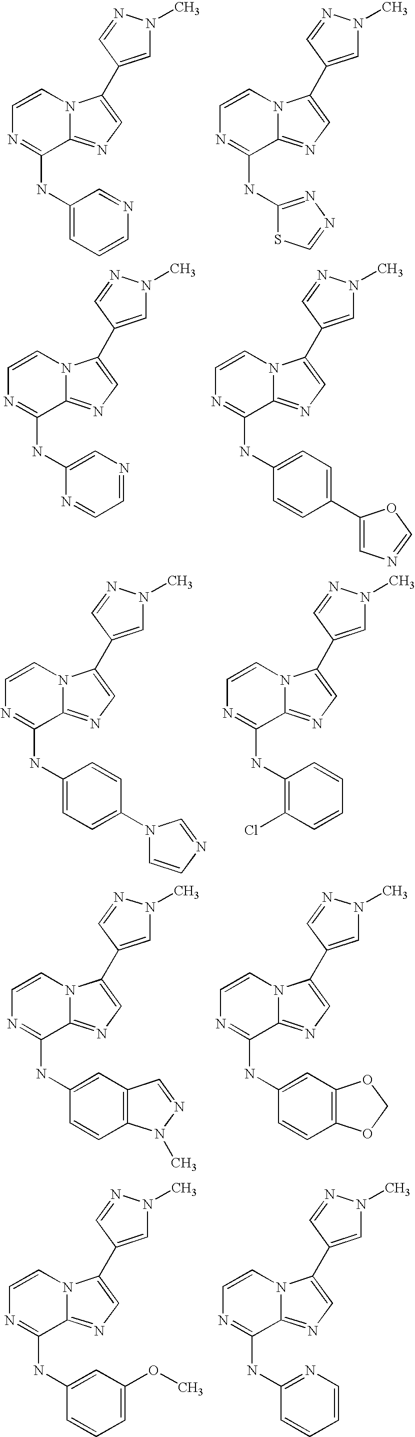 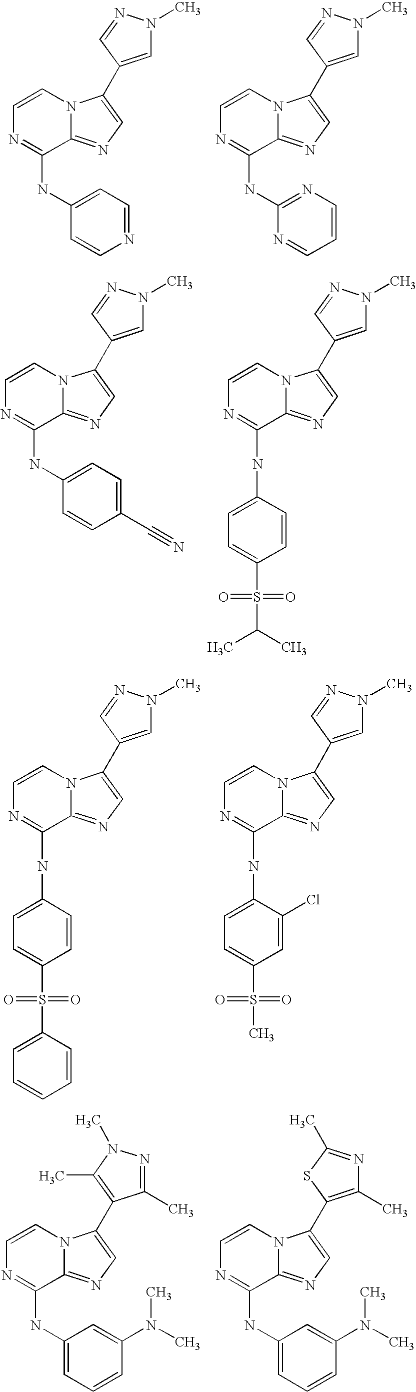 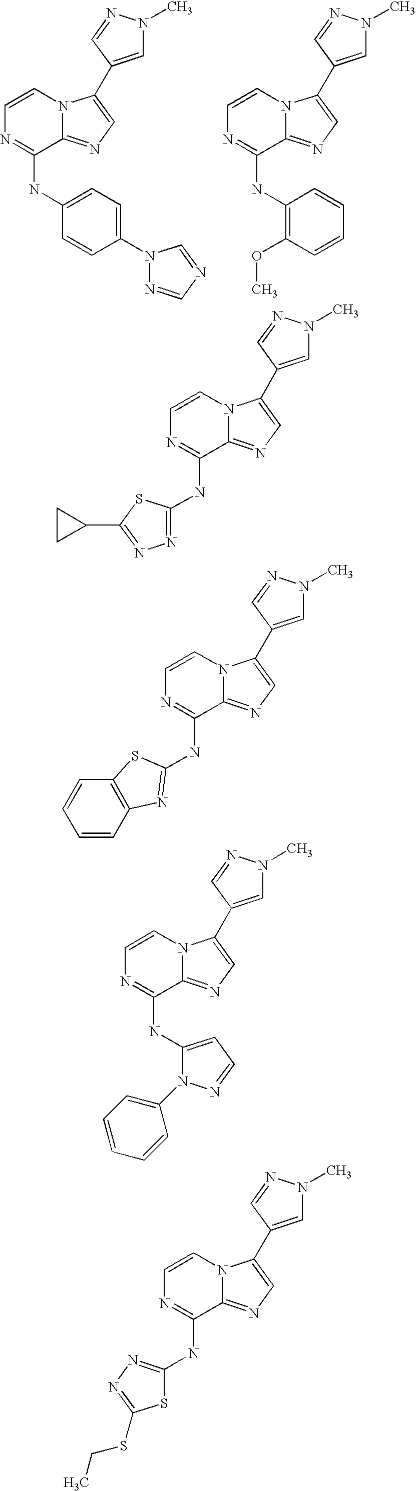 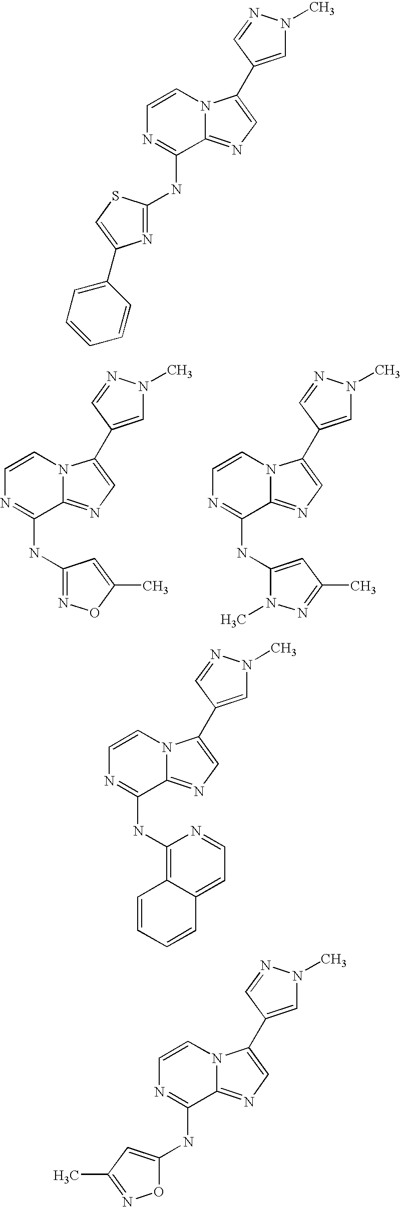 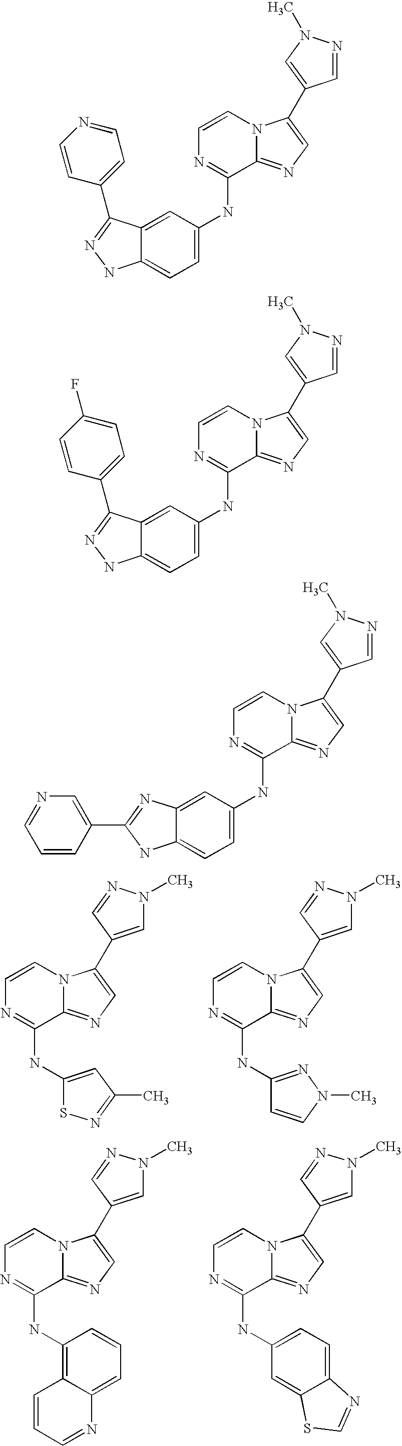 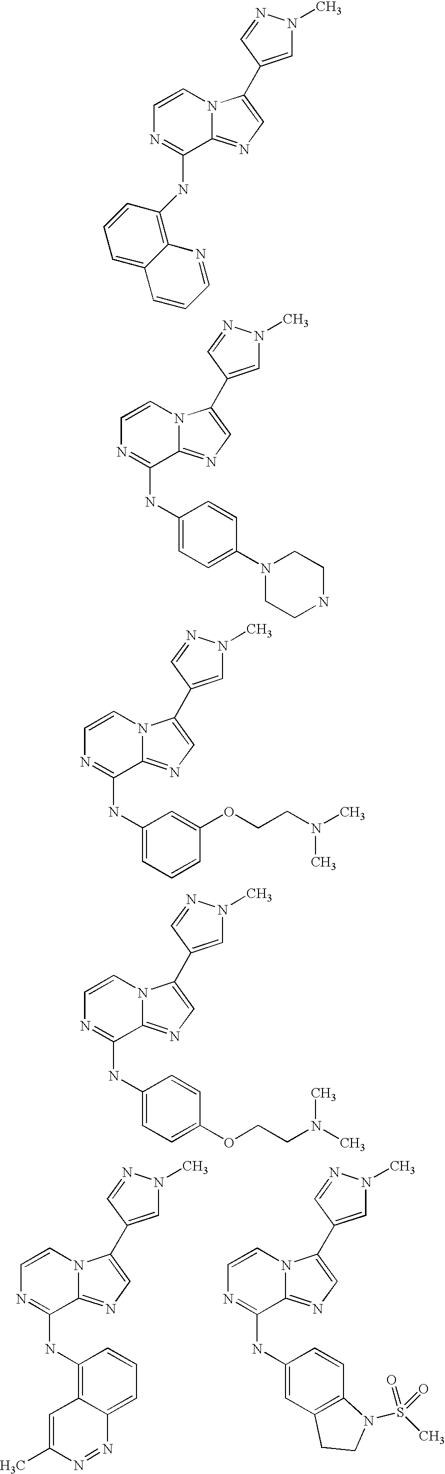 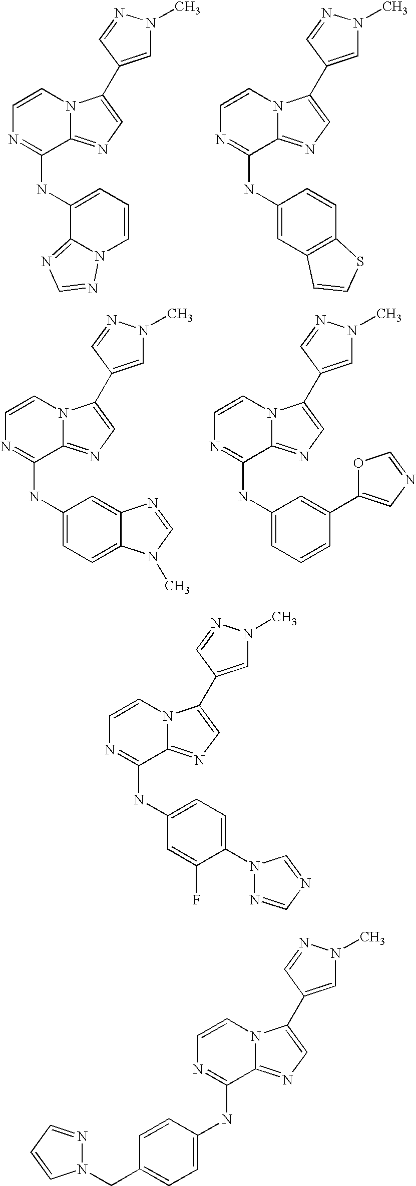 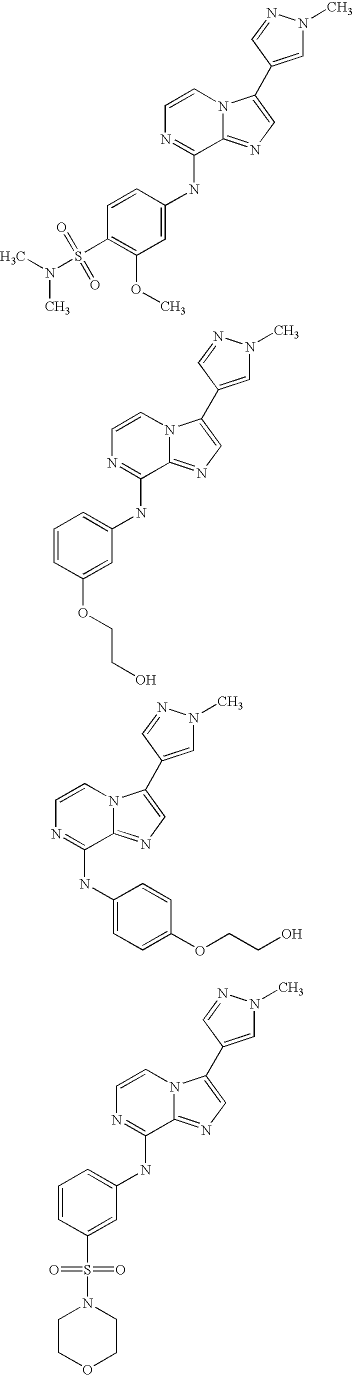 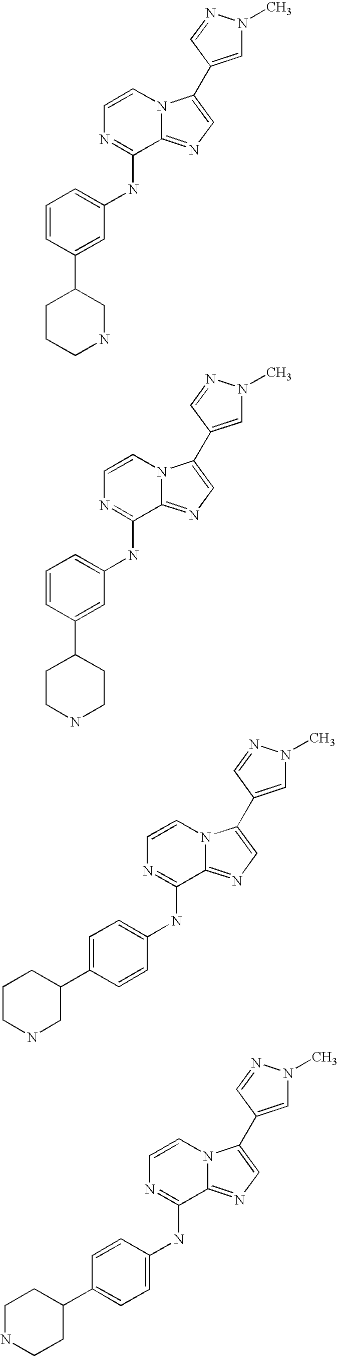 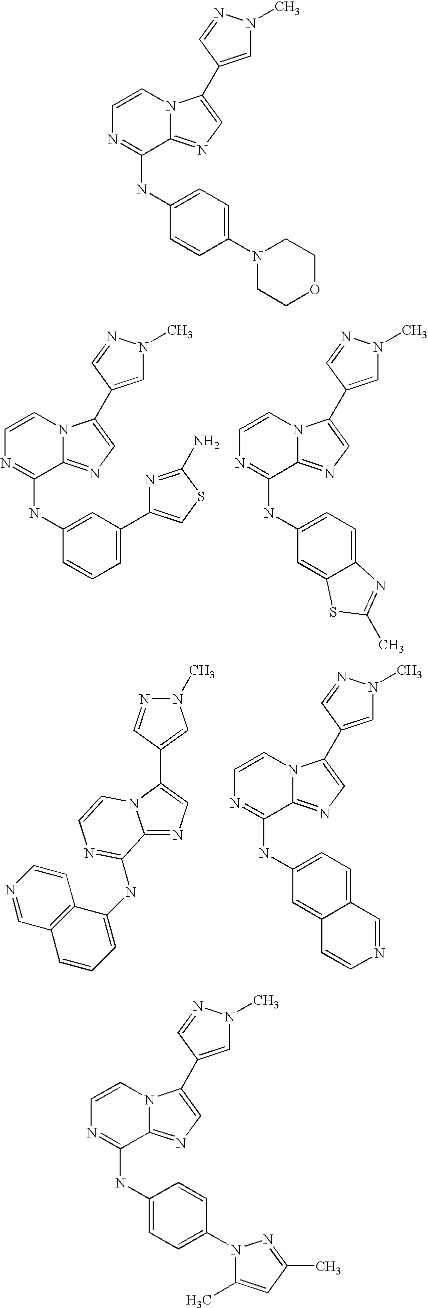 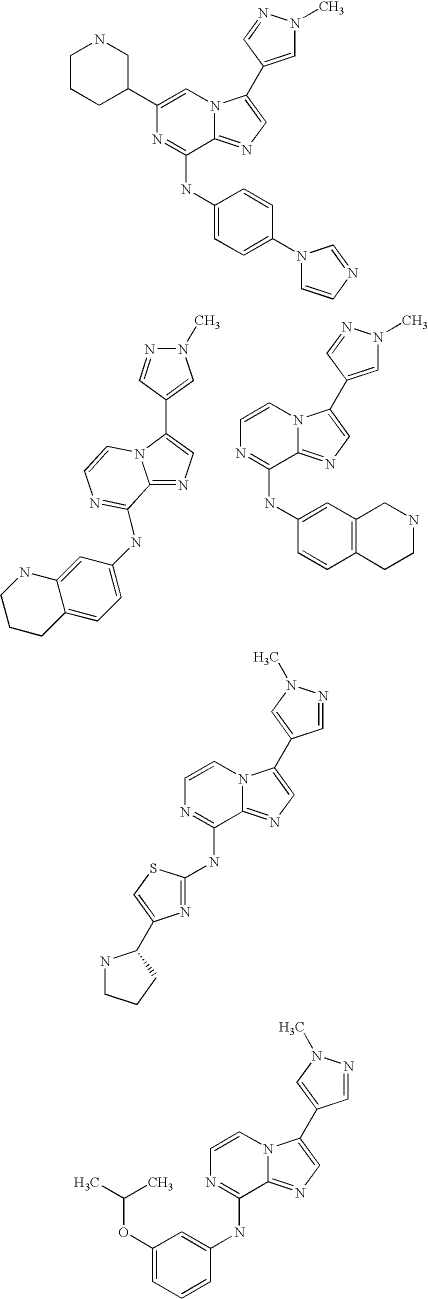 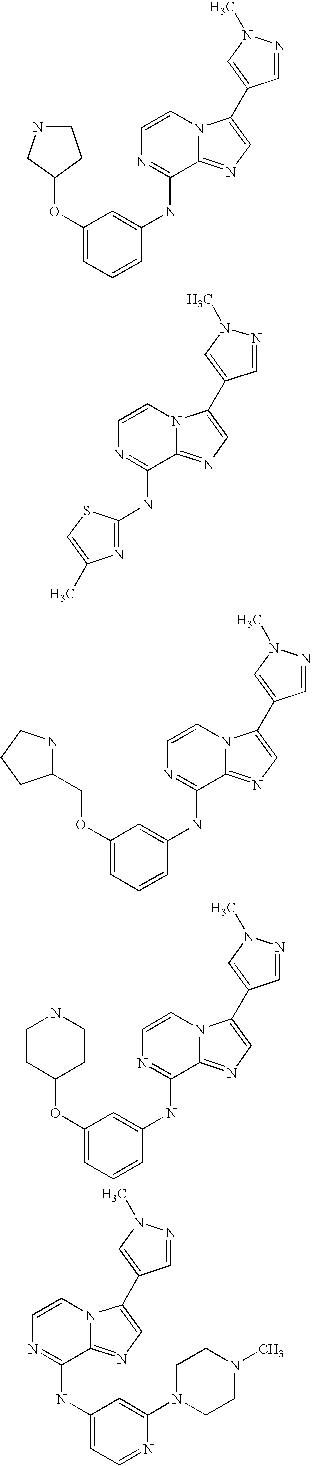 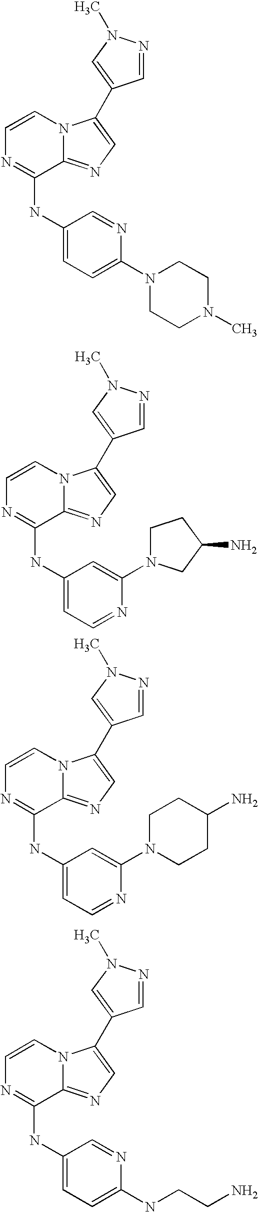 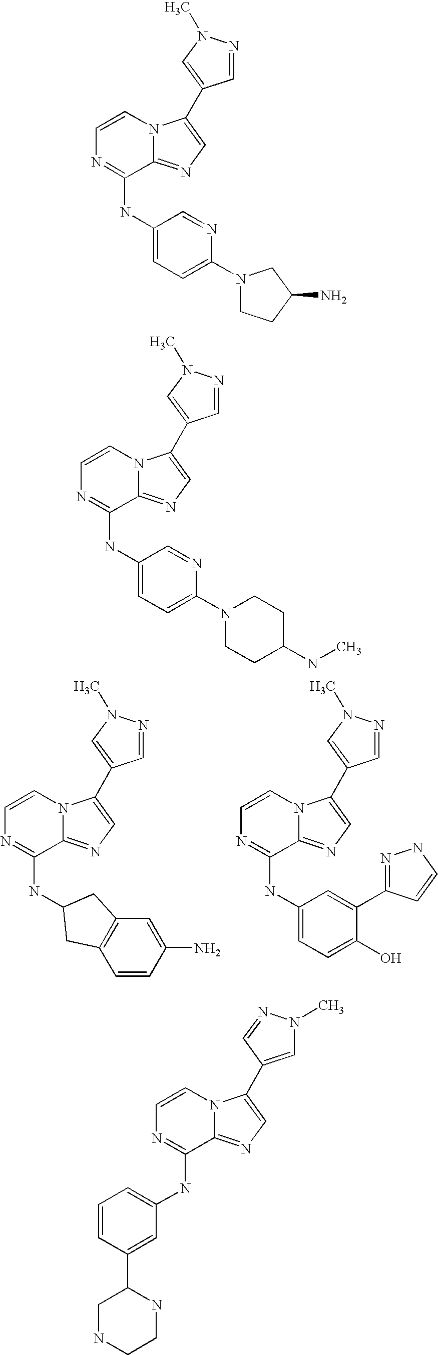 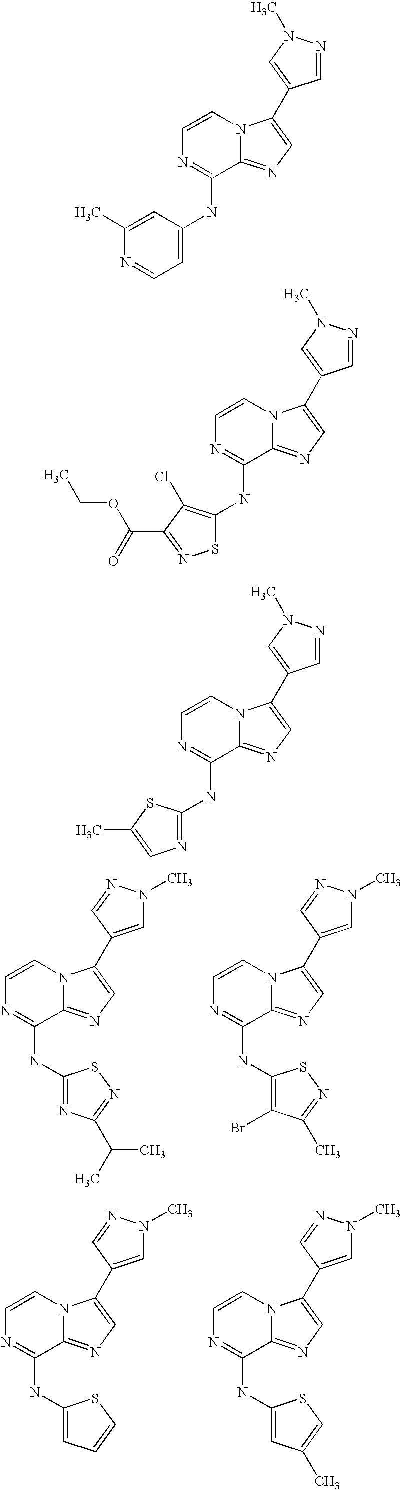 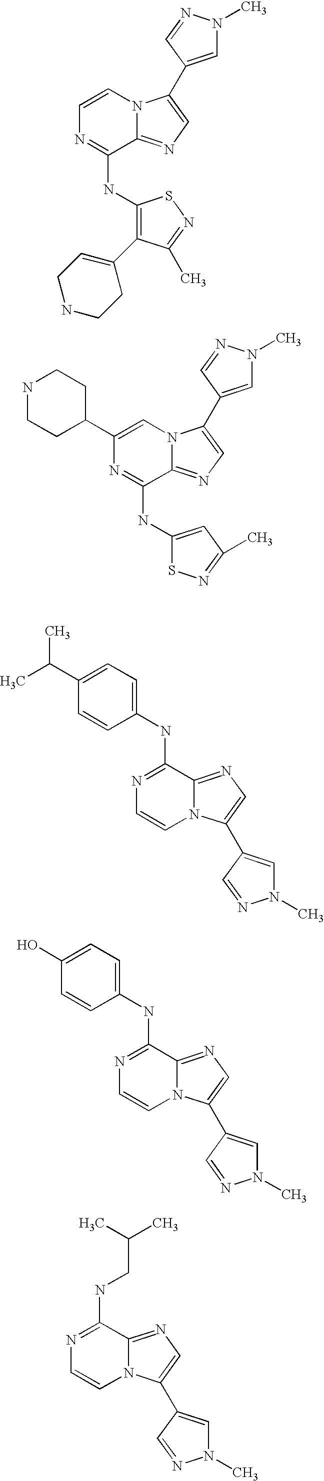 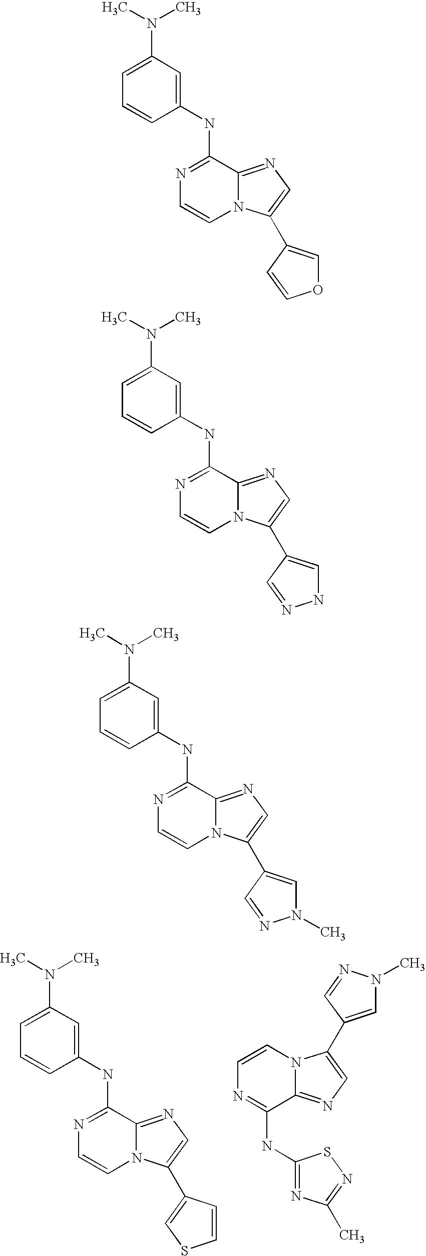 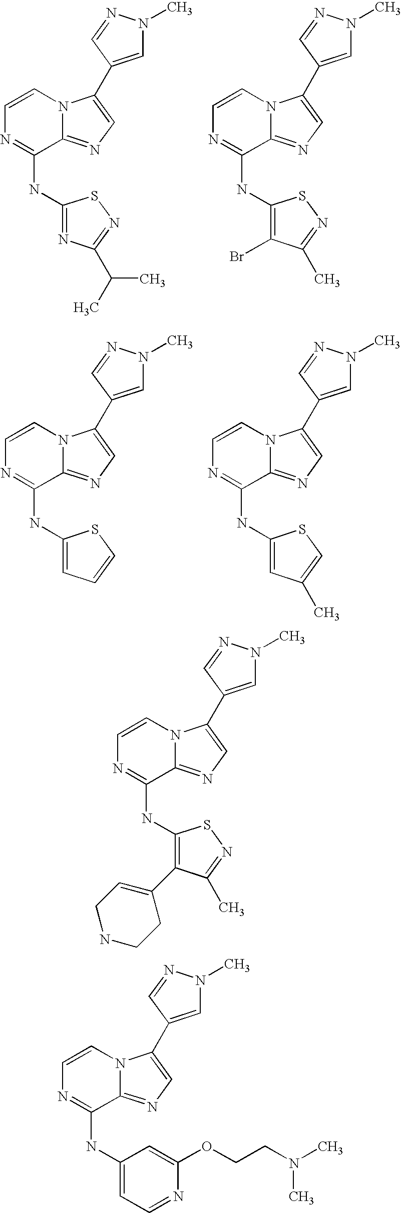 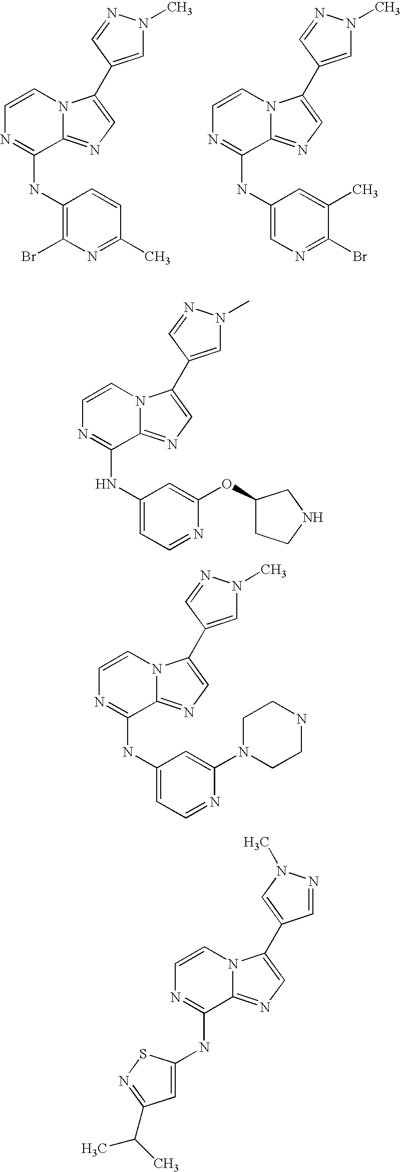 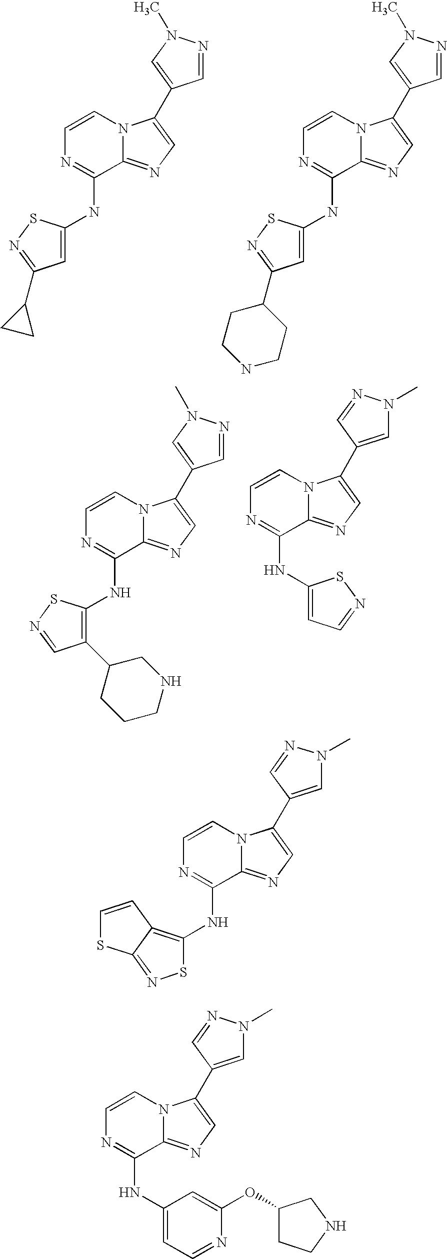 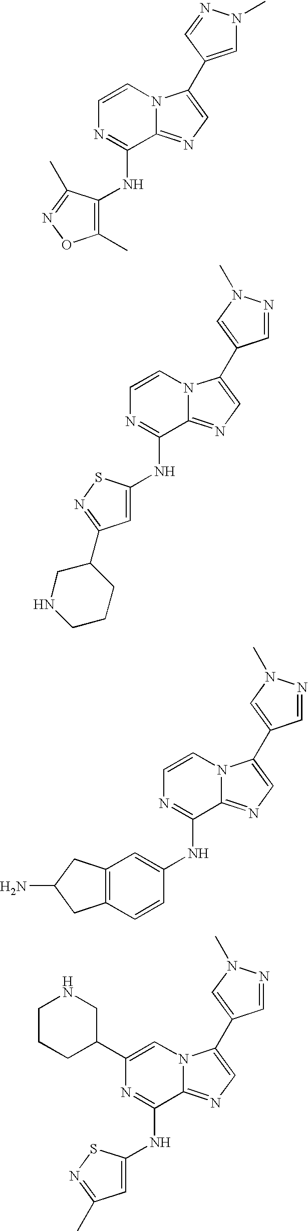 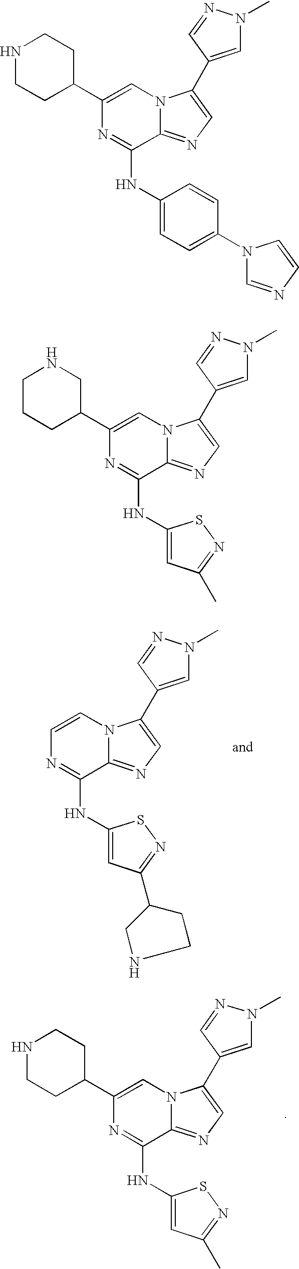
or a pharmaceutically acceptable salt, solvate, ester or prodrug thereof;
h. A compound of the formula: 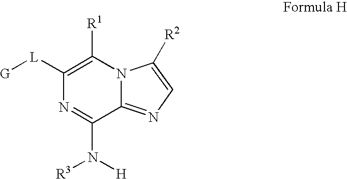
or a pharmaceutically acceptable salt, solvate, ester or prodrug thereof, wherein:
L is selected from the group consisting of S, S(O) and S(O₂);
G is alkyl, alkenyl, cycloalkyl, cycloalkenyl, aryl, heteroaryl, heterocyclenyl or heterocyclyl, wherein each of said alkyl, alkenyl, cycloalkyl, cycloalkenyl, aryl, heteroaryl, heterocyclenyl or heterocyclyl can be unsubstituted or optionally independently substituted with one or more moieties which are independently selected from the group consisting of —OR⁵, halo, —CN, —C(O)NR⁵R⁶, —N(H)—C(O)R⁵, —N(H)—C(O)—NR⁵R⁶, —S(O₂)NR⁵R⁶, —NR⁵R⁶, —C(O)R⁵, —C(O₂)R⁵, —SR⁵, —S(O)R⁵, —S(O₂)R⁵;
R¹ is H, halo, alkyl, aryl or heteroaryl, wherein each of said alkyl, aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —C(O)NR⁵R⁶ and —OR⁵;
R² is selected from the group consisting of H, R⁹, alkyl, aryl, arylalkyl, heteroaryl, heteroarylalkyl, heterocyclyl, alkenyl, alkynyl, cycloalkyl, cycloalkylalkyl, heterocyclylalkyl, —CF₃, —C(O)R⁷, 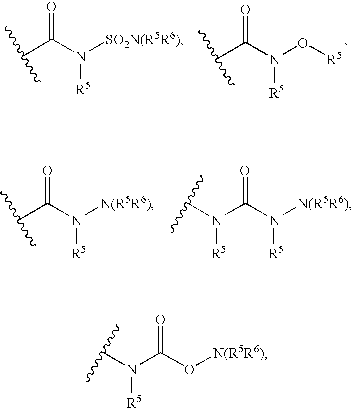
alkyl substituted with 1-6 R⁹ groups which groups can be the same or different with each R⁹ being independently selected, 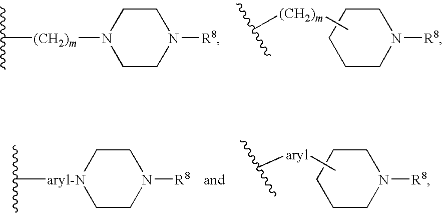
wherein each of the above-said aryl, heteroaryl, cycloalkyl, arylalkyl and heterocyclyl can be unsubstituted or optionally independently substituted with one or more moieties which can be the same or different, each moiety being independently selected from the group consisting of halo, alkyl, alkenyl, alkynyl, cycloalkyl, aryl, heteroaryl, heterocyclyl, CF₃, CN, —OCF₃, —OR⁶, —C(O)R⁷, —NR⁵R⁶, —C(O₂)R⁶,
—C(O)NR⁵R⁶, —SR⁶, —S(O₂)R⁷, —S(O₂)NR⁵R⁶, —N(R⁵)S(O₂)R⁷,
—N(R⁵)C(O)R⁷ and —N(R⁵)C(O)NR⁵R⁶;
R³ is selected from the group consisting of H, alkyl, aryl, heteroaryl, heterocyclyl, aralkyl, heteroarylalkyl, cycloalkylalkyl, heterocyclylalkyl, 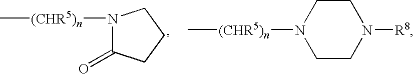
—(CHR⁵)ₙ—OR⁶, —S(O₂)R⁶, —C(O)R⁶, —S(O₂)NR⁵R⁶, —C(O)OR⁶, —C(O)NR⁵R⁶, cycloalkyl, —CH(aryl)₂, —CH(heteroaryl)₂, —(CH₂)ₙ—NR⁸, and 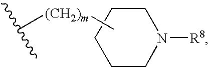
wherein each of said alkyl, aryl, heteroaryl and heterocyclyl can be substituted or optionally substituted with one or more moieties which can be the same or different, each moiety being independently selected from the group consisting of halo, alkyl, aryl, cycloalkyl, CF₃, CN, —OCF₃, —OR⁵, —NR⁵R⁶, —C(O₂)R⁵, —C(O)NR⁵R⁶, —SR⁶, —S(O₂)R⁶, —S(O₂)NR⁵R⁶, —N(R⁵)S(O₂)R⁷, —N(R⁵)C(O)R⁷ and —N(R⁵)C(O)NR⁵R⁶;
R⁵ is H, alkyl, aryl, heteroaryl, heterocyclyl or cycloalkyl; and
R⁶ is selected from the group consisting of H, alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁷ is selected from the group consisting of alkyl, aryl, heteroaryl, arylalkyl and heteroarylalkyl, wherein each of said alkyl, heteroarylalkyl, aryl, heteroaryl and arylalkyl can be unsubstituted or optionally substituted with one or more moieties which can be the same or different, each moiety being independently selected from the group consisting of halogen, alkyl, aryl, cycloalkyl, CF₃, OCF₃, CN, —OR⁵, —NR⁵R⁶, —CH₂OR⁵, —C(O₂)R⁵, —C(O)NR⁵R⁶, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
R⁸ is selected from the group consisting of R⁶, —C(O)NR⁵R⁶,
—S(O₂)NR⁵R⁶, —C(O)R⁷, —C(O₂)R⁶, —S(O₂)R⁷ and —(CH₂)-aryl;
R⁹ is selected from the group consisting of halogen, CN, NR⁵R⁶,
—C(O₂)R⁶, —C(O)NR⁵R⁶, —OR⁶, —C(O)R⁷, —SR⁶, —S(O₂)R⁷, —S(O₂)NR⁵R⁶, —N(R⁵)S(O₂)R⁷, —N(R⁵)C(O)R⁷ and —N(R⁵)C(O)NR⁵R⁶;
m is 0 to 4; and
p is 0-3;
i. A compound of Formula I: 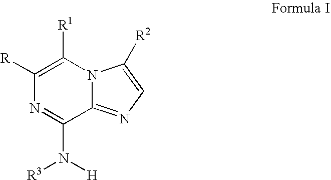
or a pharmaceutically acceptable salt, solvate, ester or prodrug thereof, wherein:
R is H, CN, —NR⁵R⁶, cycloalkyl, cycloalkenyl, heterocyclenyl, heteroaryl, —C(O)NR⁵R⁶, —N(R⁵)C(O)R⁶, heterocyclyl, heteroaryl substituted with (CH₂)₁₋₃NR⁵R⁶, unsubstituted alkyl, or alkyl substituted with one or more moieties which can be the same or different each moiety being independently selected from the group consisting of —OR⁵, heterocyclyl, —N(R⁵)C(O)N(R⁵R⁶), —N(R⁵)—C(O)OR⁶, —(CH₂)₁₋₃—N(R⁵R⁶) and —NR⁵R⁶;
R¹ is H, halo, aryl or heteroaryl, wherein each of said aryl and heteroaryl can be unsubstituted or substituted with one or more moieties which can be the same or different each moiety being independently selected from the group consisting of halo, alkyl, alkenyl, alkynyl, cycloalkyl, aryl, heteroaryl, heterocyclyl, —CH₂OR⁵, —C(O)NR⁵R⁶, —C(O)OH, —C(O)NH₂, —NR⁵R⁶ (wherein the R⁵ and R⁶, together with the N of said —NR⁵R⁶, form a heterocyclyl ring), —S(O)R⁵, —S(O₂)R⁵, —CN, —CHO, —SR⁵, —C(O)OR⁵, —C(O)R⁵ and —OR⁵;
R² is H, halo, aryl, arylalkyl or heteroaryl, wherein each of said aryl, arylalkyl and heteroaryl can be unsubstituted or optionally independently be substituted with one or more moieties which can be the same or different each moiety being independently selected from the group consisting of halo, amide, alkyl, alkenyl, alkynyl, cycloalkyl, aryl, —C(O)OH, —C(O)NH₂, —NR⁵R⁶ (wherein the R⁵ and R⁶, together with the N of said —NR⁵R⁶, form a heterocyclyl ring), —CN, arylalkyl, —CH₂OR⁵, —S(O)R⁵, —S(O₂)R⁵, —CN, —CHO, —SR⁵, —C(O)OR⁵, —C(O)R⁵, heteroaryl and heterocyclyl;
R³ is heterocyclyl-(CR⁷R⁸)ₙ—X, heterocyclenyl-(CR⁷R⁸)ₙ—X, heteroaryl-(CR⁷R⁸)ₙ—X or aryl-(CR⁷R⁸)ₙ—X wherein each of the heterocyclyl-, heterocyclenyl-, heteroaryl- or aryl-moieties of said R³ can be unsubstituted or substituted with one or more moieties, independently selected from the group consisting of —CONR⁵R⁶, —OR⁵ and alkyl, n is 1-6,
X is selected from the group consisting of, —NR⁵R⁶, —OR⁵, —SO—R⁵ and —SR⁵, R⁷ and R⁸ are each independently hydrogen or alkyl;
R⁵ is selected from the group consisting of hydrogen, alkyl, alkenyl, alkoxyalkyl, -alkyl-S-alkyl, aminoalkyl, aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl, alkylN(alkyl)₂, alkylNH(alkyl), alkylN(alkenyl)2, -alkylN(alkoxyl)₂, -alkyl-SH and hydroxyalkyl, wherein each of said aryl, heteroaryl, heterocyclenyl, heterocycloalkyl, cycloalkyl, cycloalkenyl, heterocyclylalkoxyl, —S-alkylheterocyclyl, heterocyclyl, heterocyclenyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
R⁶ is selected from the group consisting of hydrogen, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl, wherein each of said aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl can be unsubstituted or substituted with one or more moieties independently selected from the group consisting of alkyl, alkyl, alkenyl, aryl, cycloalkenyl, cycloalkyl, arylalkyl, cycloalkenylalkyl, cycloalkylalkyl, heteroaryl, heterocyclenyl, heterocyclyl, heteroarylalkyl, heterocyclenylalkyl, heterocycloalkylalkyl, alkoxyalkyl, -alkyl-S-alkyl, -alkyl SH, alkoxyl, —S-alkyl, hydroxyalkyl, and aminoalkyl;
further wherein in any —NR⁵R⁶ in Formula I, said R⁵ and R⁶ can optionally be joined together with the N of said —NR⁵R⁶ to form a cyclic ring or bridged cyclic ring, wherein each of said cyclic ring or bridged cyclic ring, can be unsubstituted or substituted with one or more moieties, which can be the same or different, independently selected from the group consisting of hydroxyl, —SH, alkyl, alkenyl, hydroxyalkyl, -alkyl-SH, alkoxyl, —S-alkyl, —CO₂-alkyl, —CO₂-alkenyl, arylalkyl, cycloalkenylalkyl, cycloalkylalkyl, heteroarylalkyl, heterocyclenylalkyl, heterocycloalkylalkyl, heteroaryl, aryl, cycloalkenyl, cycloalkyl, spiroheterocyclyl, spiroheterocyclenyl, spiroheteroaryl, spirocyclyl, spirocyclenyl, spiroaryl, alkoxyalkyl, -alkyl-S-alkyl, heterocyclyl, and heterocyclenyl;
j. A compound having the formula: 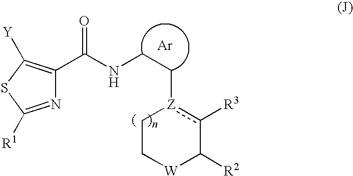
or a pharmaceutically acceptable salt, solvate, ester, prodrug or stereoisomer thereof, wherein the dashed lines indicate an optional and additional bond, and wherein:
R¹ is nitrogen-containing heteroaryl, nitrogen-containing heterocyclyl, nitrogen-containing benzofused heteroaryl or nitrogen-containing benzofused heterocyclyl, wherein R¹ is joined to the rest of the compound of formula (I) via a ring nitrogen atom, and wherein one or more ring carbon atoms of a nitrogen-containing heteroaryl, nitrogen-containing heterocyclyl, nitrogen-containing benzofused heteroaryl or nitrogen-containing benzofused heterocyclyl group can be substituted with up to 5 substituents, which may be the same or different, and are independently selected from alkyl, aryl, halo, —OH, —O-alkyl, —O-aryl, —N(R⁸)₂, —CF₃, —NO₂, —C(O)R⁸, —C(O)OR⁸, —C(O)N(R⁸)₂, —OC(O)R⁸ or —NHC(O)R⁸;
R² is —H, -alkyl, —NH₂ or —CH₂NH₂;
R³ is —H, -alkyl, —NH₂ or —CH₂NH₂;
each occurrence of R⁴ is independently —H, -alkyl, —NH₂, —OH, -alkylene-OH, —CH₂NH₂, —C(O)R⁵, —C(O)NH₂, —C(O)NH-alkyl, —C(O)N(alkyl)₂, —NHC(O)R⁶ or —NHS(O)₂R⁶;
R⁵ is —H, -alkyl, -aryl, -heteroaryl, —NHOH;
R⁶ is —H, -alkyl or —CF₃;
R⁷ is —H, —OH, —C₁-C₆ alkyl, —O—(C₁-C₆ alkyl), or —CF₃;
R⁸ is —H, alkyl, aryl, heterocyclyl, heteroaryl or cycloalkyl;
Ar is -arylene- or -heteroarylene-, wherein the arylene or heteroarylene is joined via 2 of its adjacent ring carbon atoms, and wherein the -arylene- or -heteroarylene- can be substituted with up to 4 substituents, which may be the same or different, and are independently selected from -halo, alkyl, alkoxy, aryloxy, —SR⁸, —S(O)R⁸, —S(O)₂R⁸, —C(O)R⁸, —C(O)OR⁸, —C(O)N(R⁸)₂, —NHC(O)R⁸, —CF₃, —CN or NO₂, and such that when Ar is tetrahydronaphthylene, R¹ and R² cannot both be hydrogen
W is —NH— or —C(R⁴)₂—, wherein both R⁴ groups and the carbon atom to which they are attached can combine to form a five to seven membered heterocyclyl or heteroaryl group;
Y is —H, -halo, -alkyl or —CN;
Z is —CR⁷— or —N—, when the optional additional bond is absent, and —C— when the optional additional bond is present;
n is an integer ranging from 0 to 2; and
(k) A compound of Formula K: 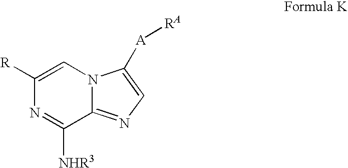
or a pharmaceutically acceptable salt, solvate, ester, prodrug or stereoisomer thereof, wherein:
R is H, halo or alkyl;
R³ is heteroaryl-X, wherein X is heterocyclylalkyl- wherein said heterocyclyl can be unsubstituted or optionally substituted with 1-4 alkyl moieties;
A is -aryl-, -heteroaryl-, —N(R¹)-aryl- or —N(R¹)-heteroaryl-, wherein each of said aryl and heteroaryl can be independently unsubstituted or optionally substituted with one or more substituents, which substituents can be the same or different, each substituent being independently selected from the group consisting of alkyl, —NO₂, halo, hydroxy, trihaloalkyl, alkoxy, and dialkylamino;
R^{A} is —(CH₂)₁₋₄-heteroaryl, 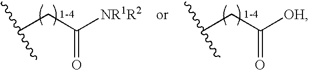
wherein said heteroaryl can optionally be fused with an aryl, wherein each of said aryl and heteroaryl can independently be optionally substituted with one or more moieties each moiety being independently selected from the group consisting of trihaloalkyl, —NO₂, halo, hydroxyalkyl, alkoxyalkyl and dialkylamino;
R¹ is H or alkyl;
R² is H, hydroxyalkyl-, arylalkyl-, heteroaryl, aryl, heteroarylalkyl-, alkyl, dialkylaminoalkyl-, alkylaminoalkyl-, cycloalkylalkyl-, cycloalkyl, heterocyclylalkyl- or heterocyclyl, wherein said aryl and aryl of arylalkyl can be unsubstituted or substituted with one or more moieties independently selected from the group consisting of trihaloalkyl, —NO₂, halo, hydroxyalkyl, alkoxyalkyl, dialkylamino and heterocyclylalkyl-, wherein said heterocyclylalkyl can be unsubstituted or substituted with alkyl or —SO₂NH₂; said heteroaryl and heteroaryl of heteroarylalkyl can be unsubstituted or substituted with one or more moieties, each moiety being independently selected from the group consisting of hydroxyalkyl, alkoxy, alkyl, halo, hydroxyl, and —NO₂; and said cycloalkyl is unsubstituted or substituted with hydroxyl; or
R¹ and R² together with the N to which each is attached, form a heterocyclic group selected from the group consisting of 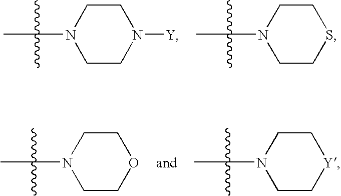
wherein
Y is alkoxyalkyl, hydroxyalkyl, dialkylaminoalkyl or alkyl, further wherein Y′ is hydroxyl.

12. The pharmaceutical composition of claim 1, wherein said KSP inhibitor represented by Formulas A-D are selected from the group consisting of: 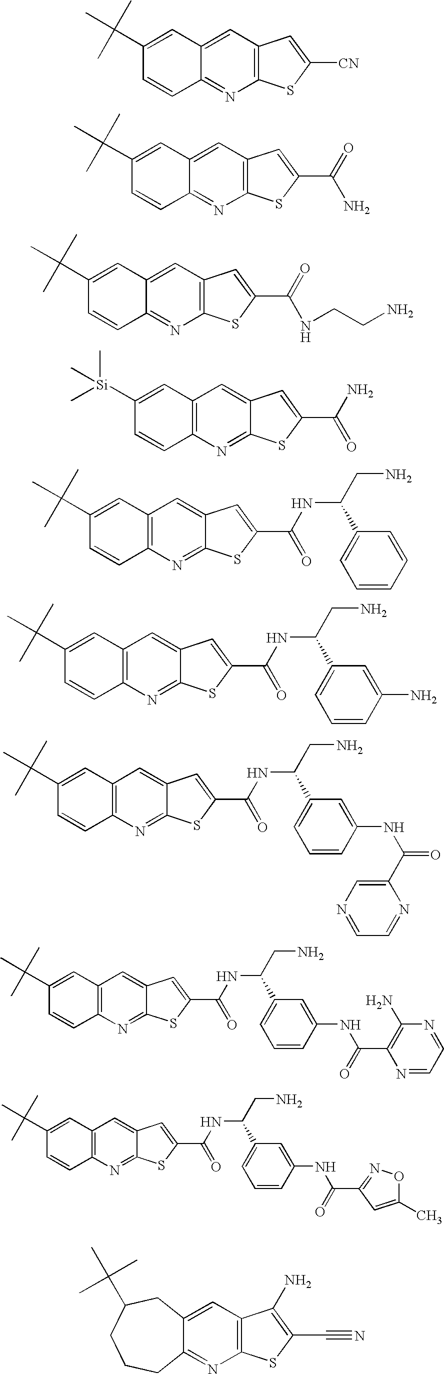 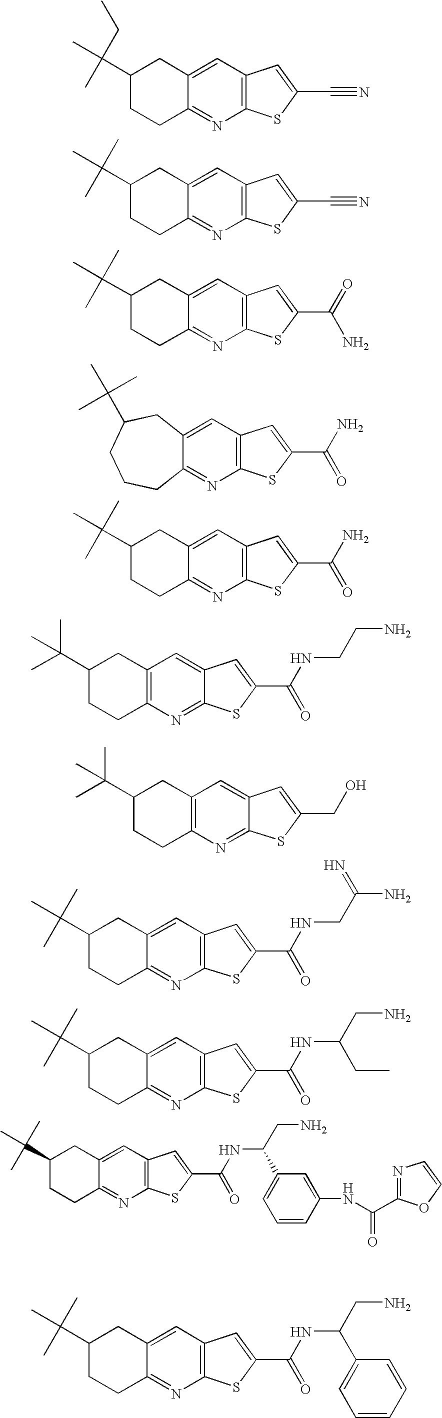 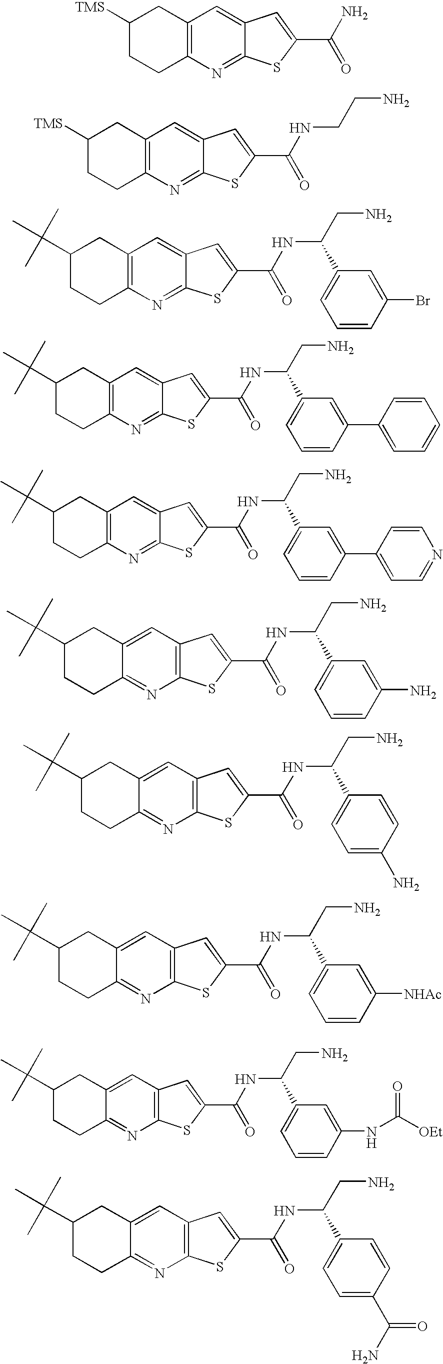 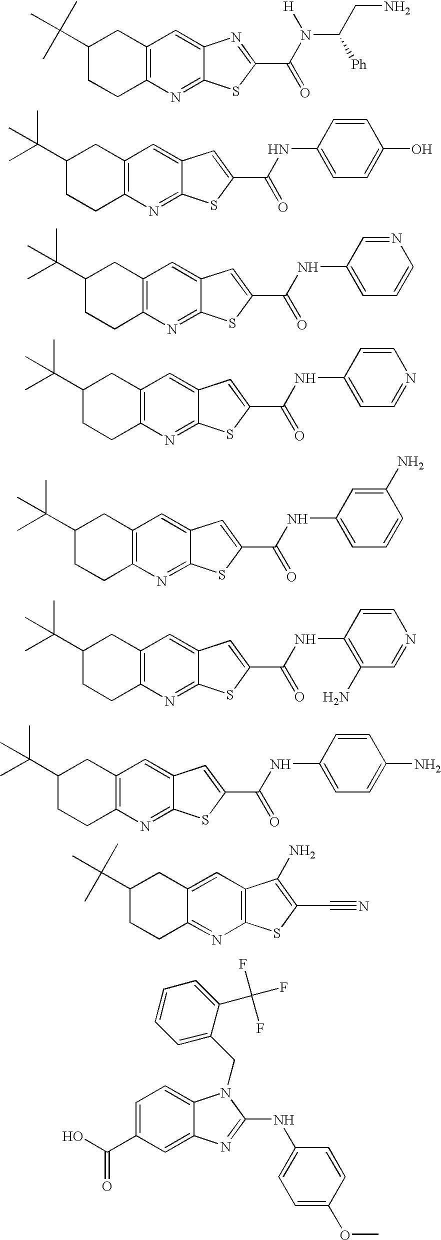 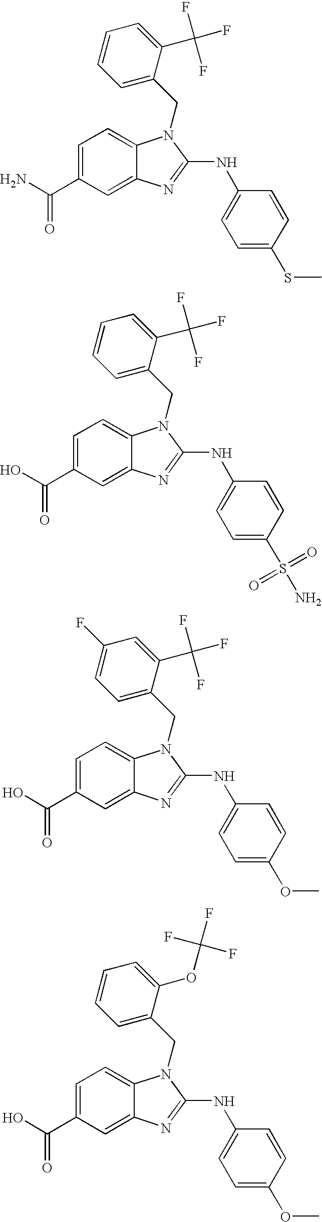 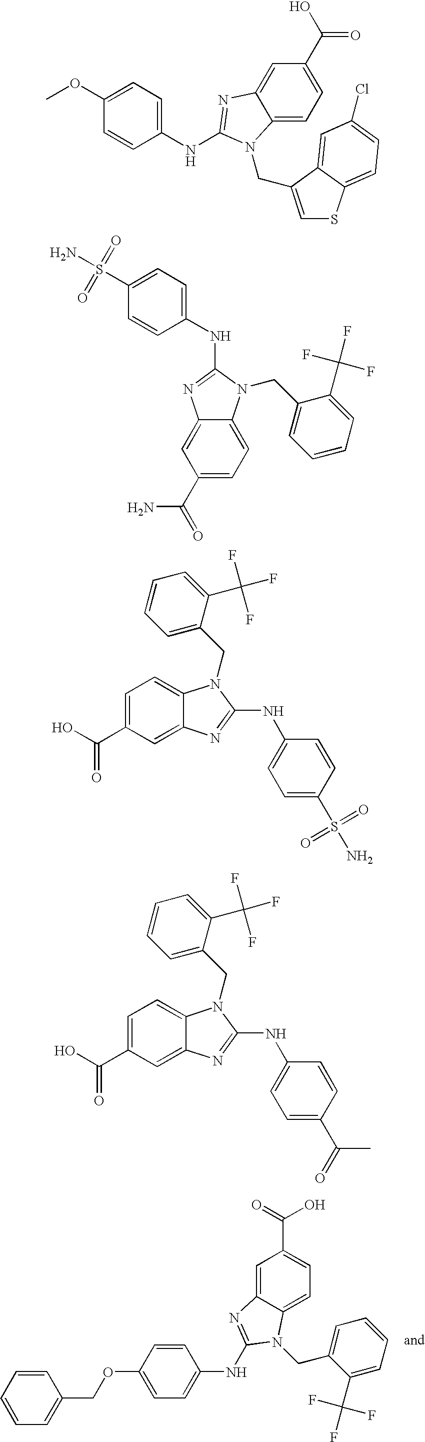 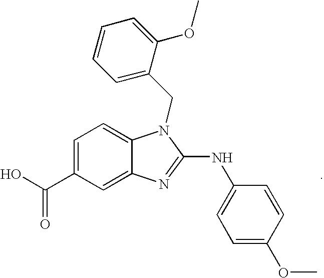
or a pharmaceutically acceptable salt, solvate, ester or prodrug thereof.

13. The method of claim 2, wherein said KSP inhibitor represented by Formulas A-D are selected from the group consisting of: 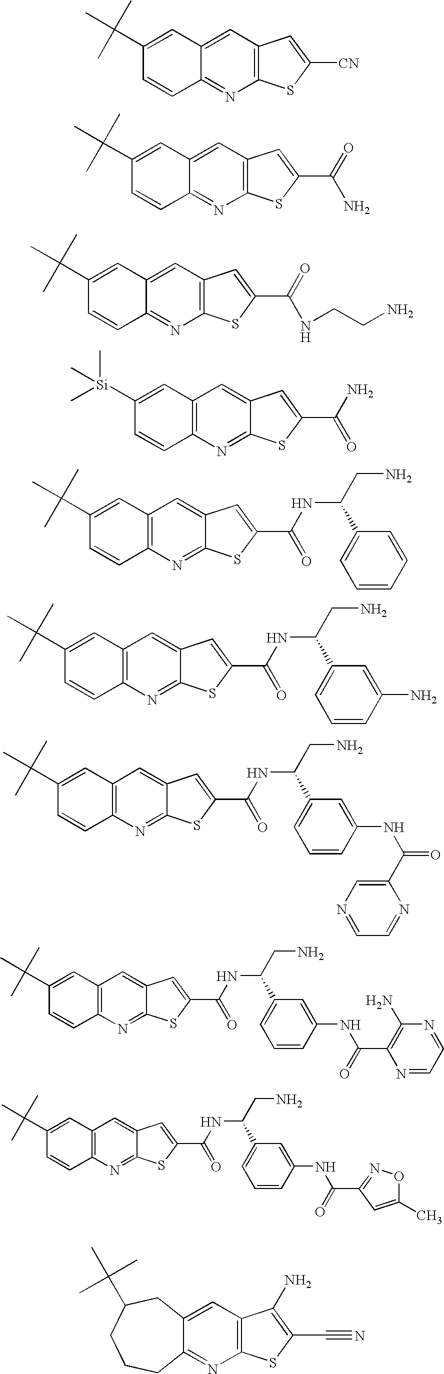 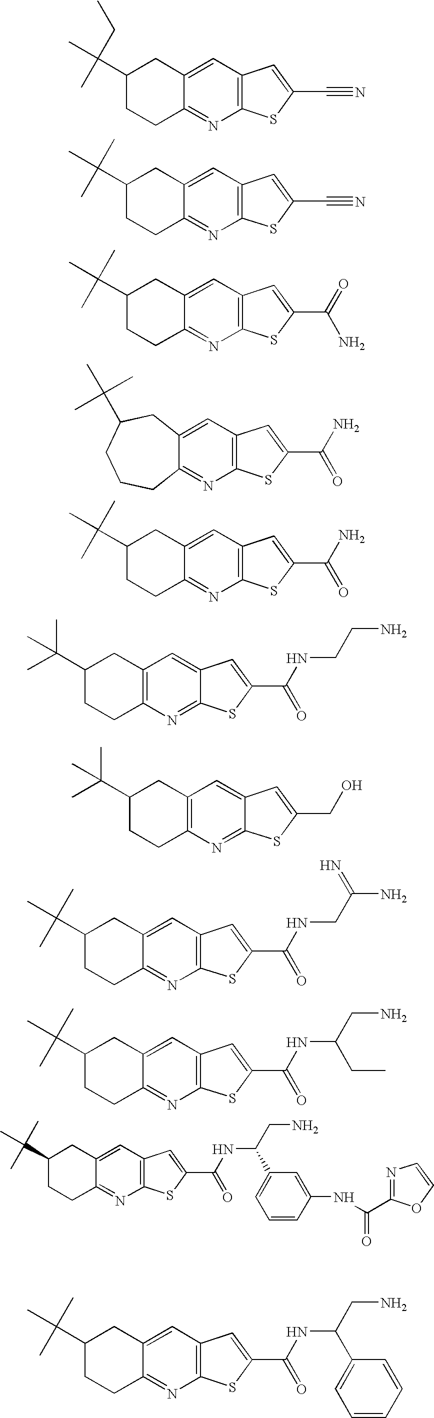 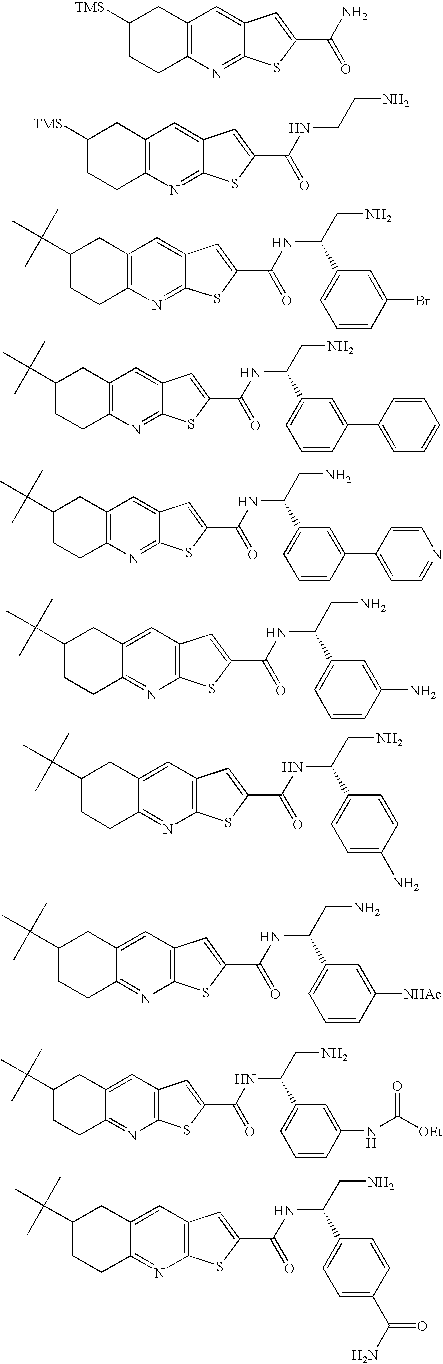 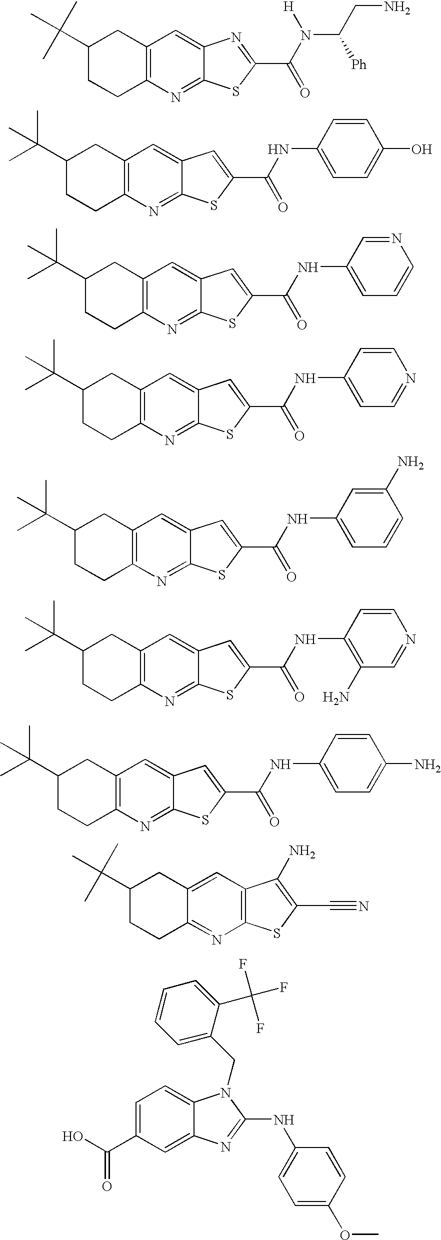 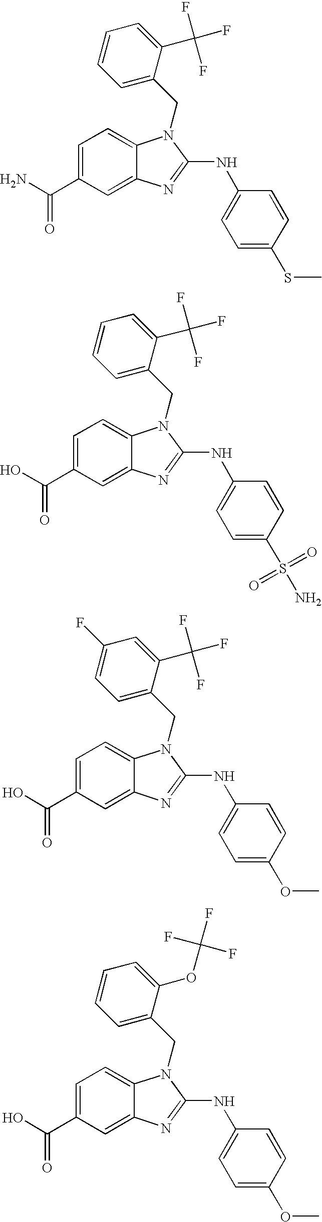 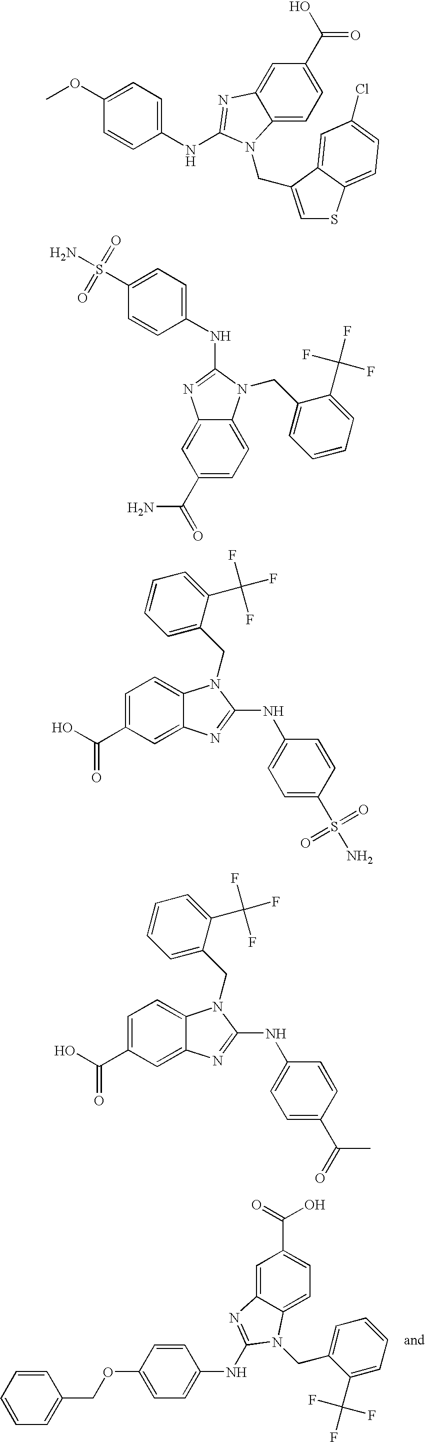 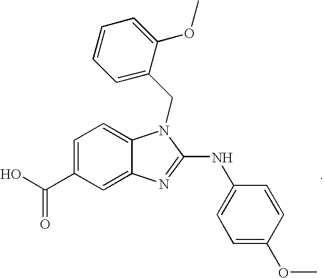
or a pharmaceutically acceptable salt, solvate, ester or prodrug thereof.

14. The pharmaceutical composition of claim 1, wherein said aurora kinase inhibitor, represented by Formulas E-K, are selected from the group consisting of: 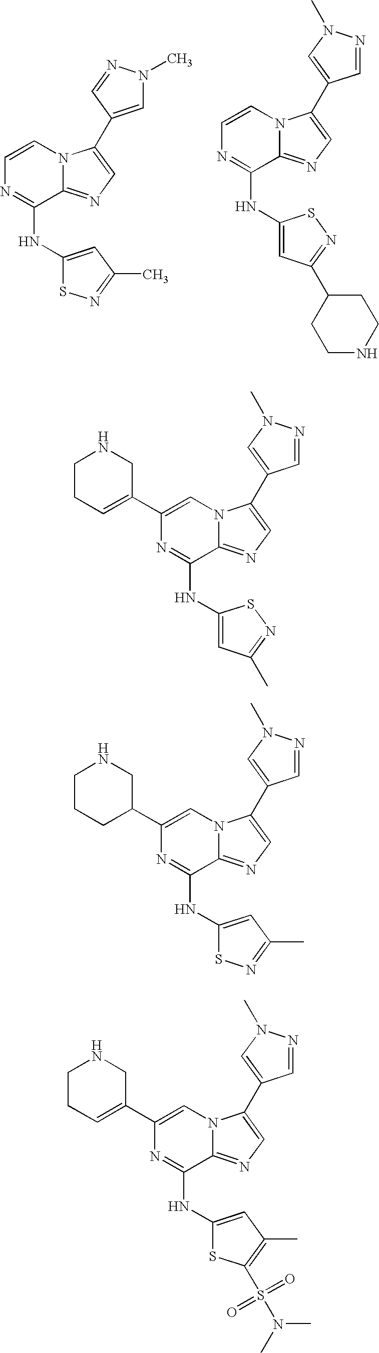 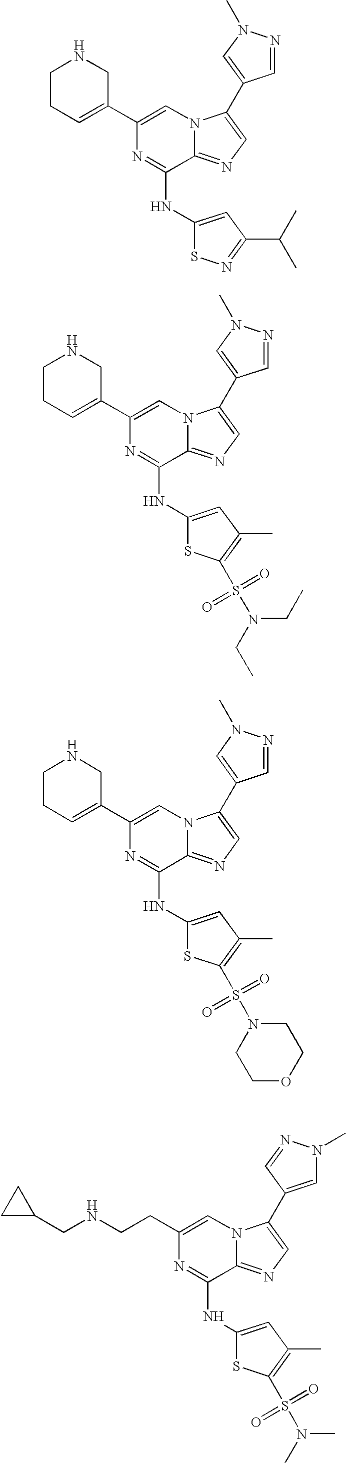 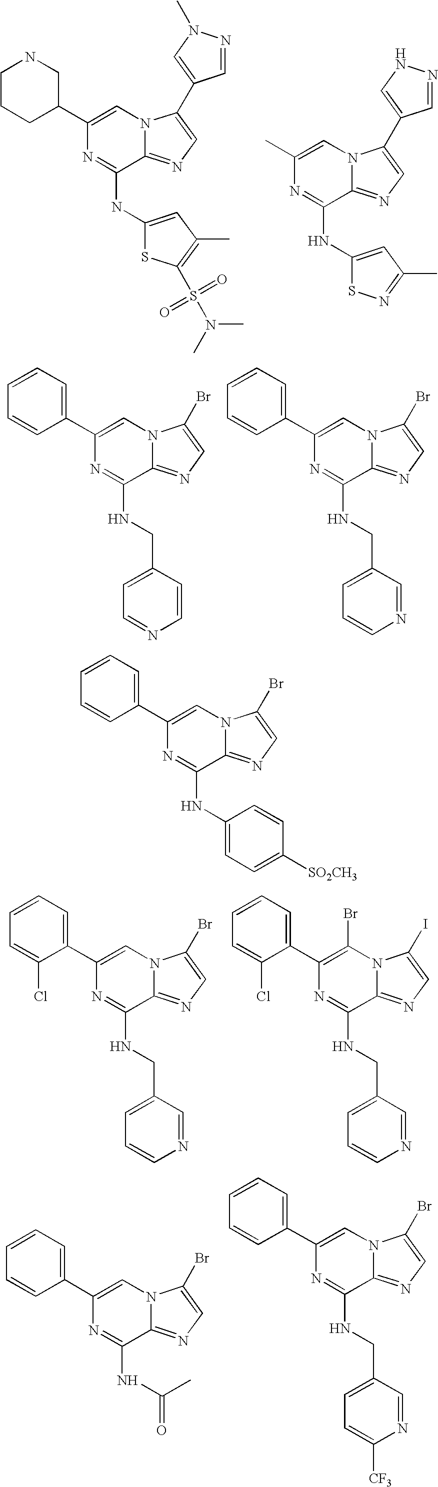 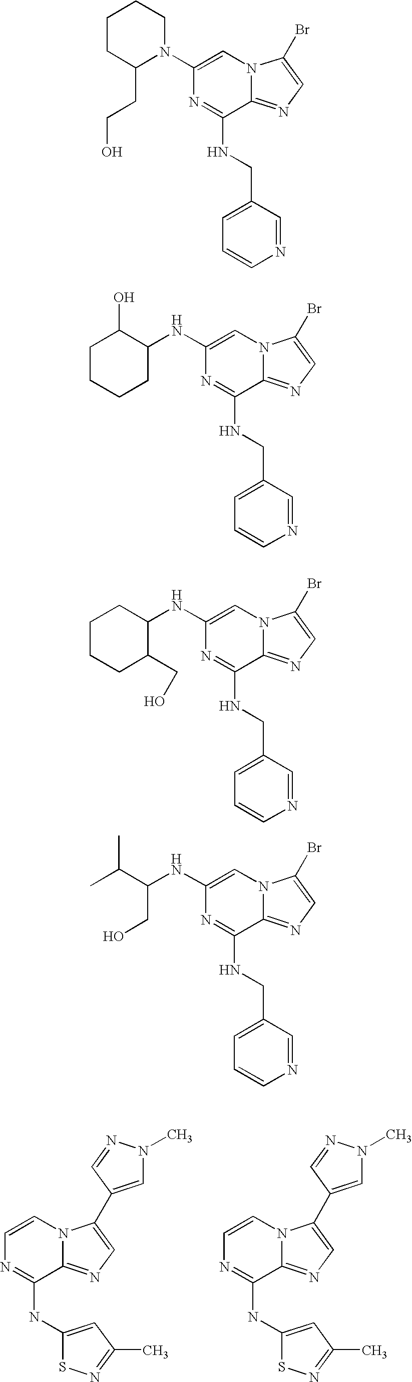 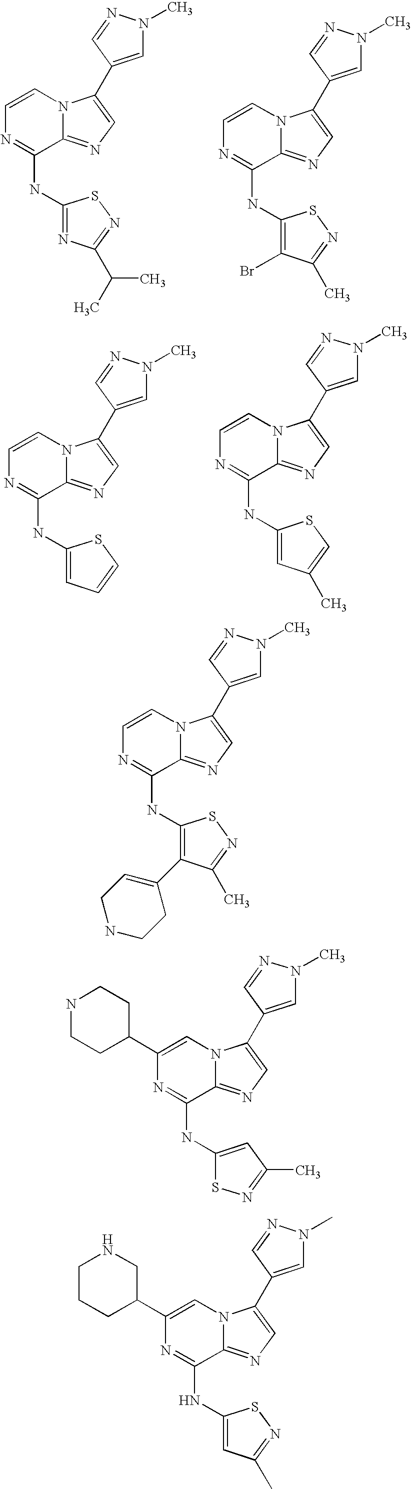 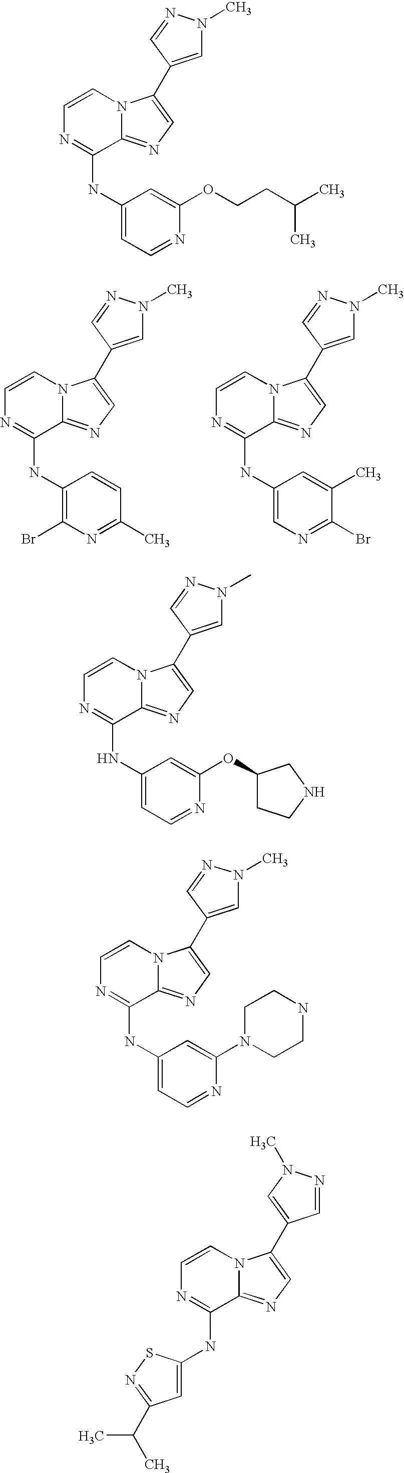 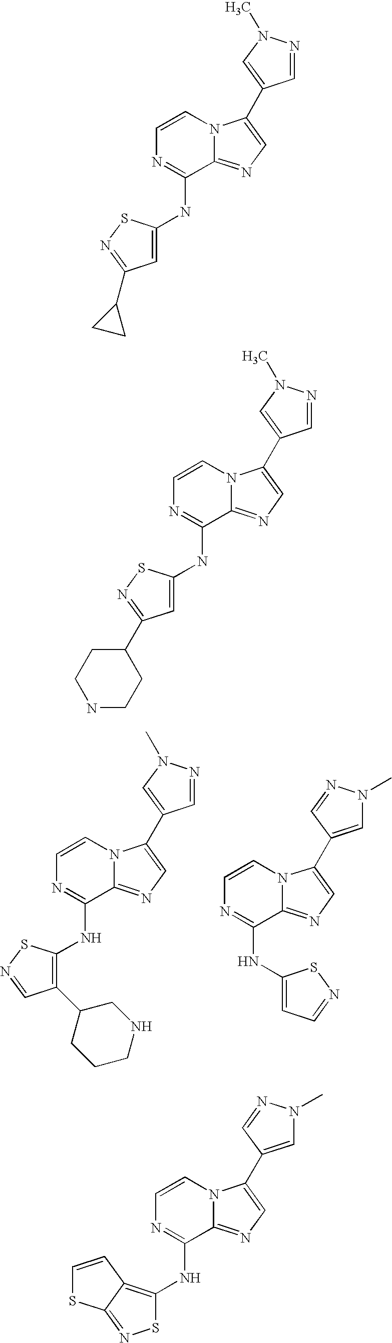 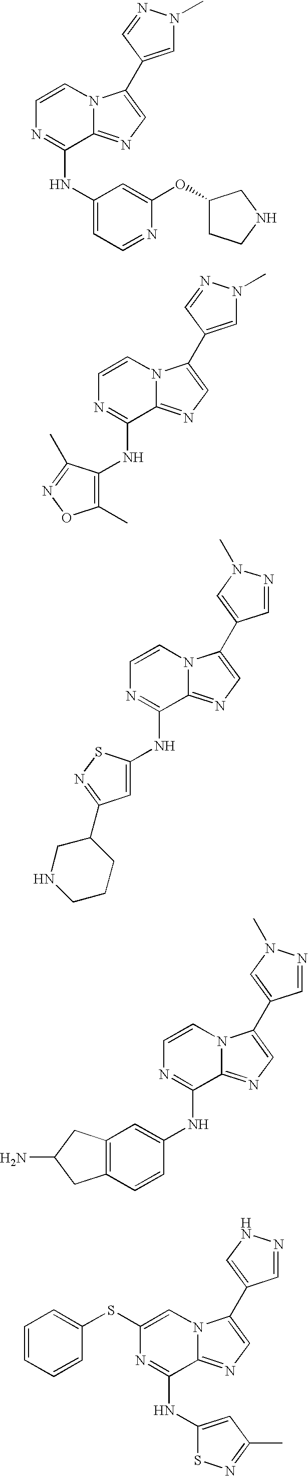 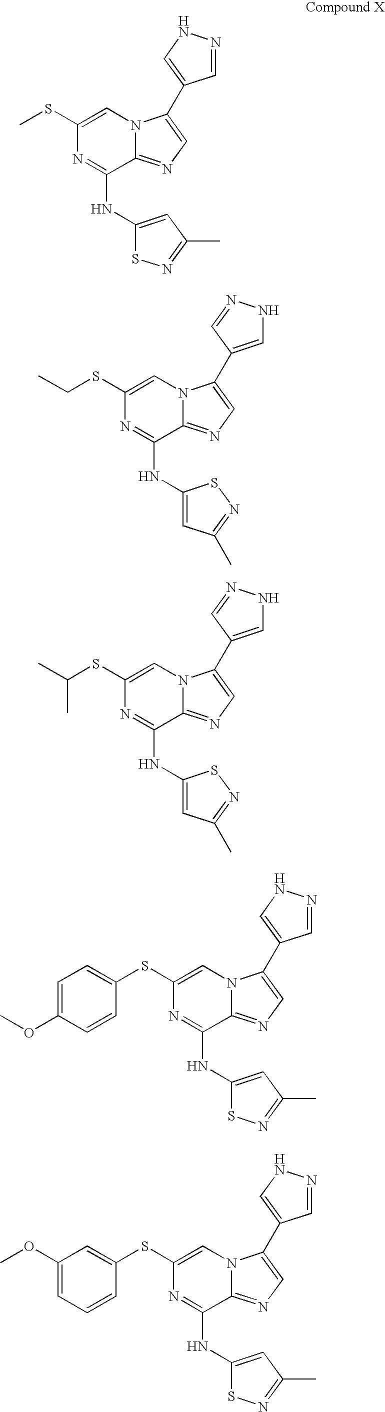 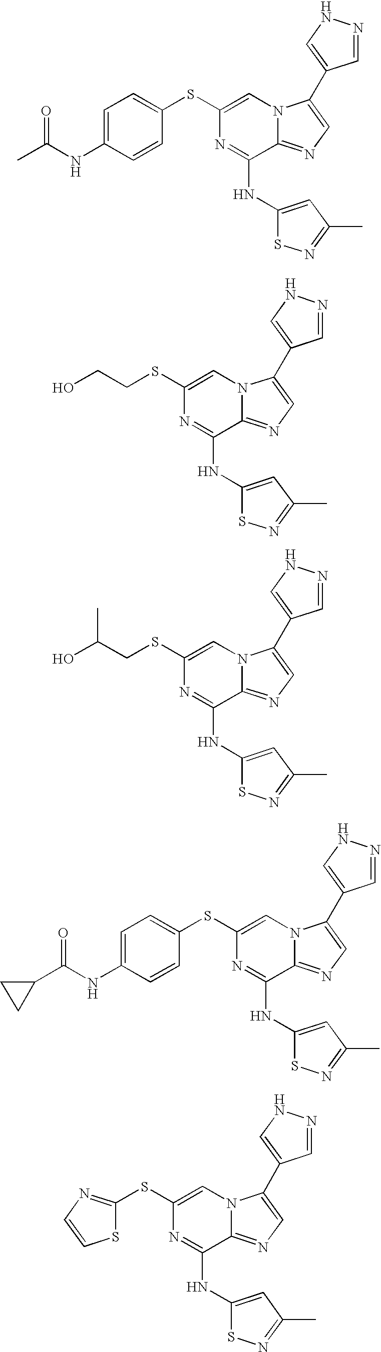 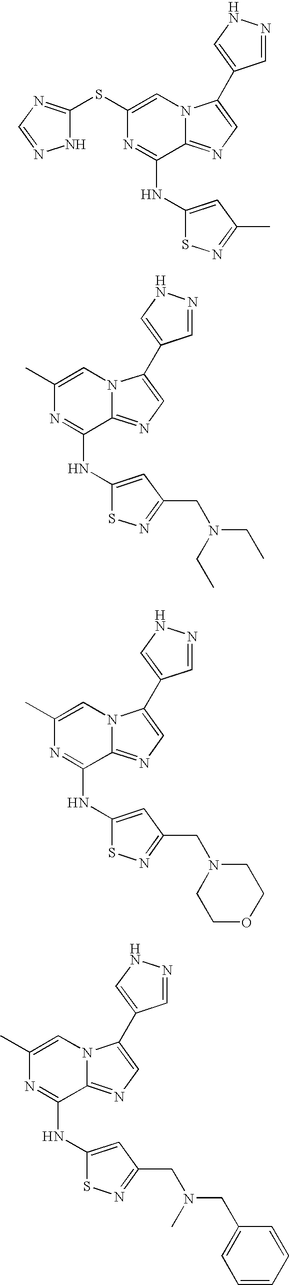 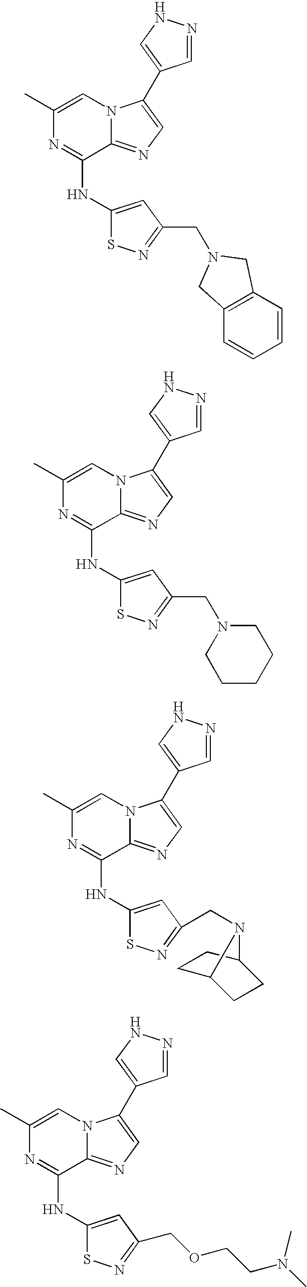 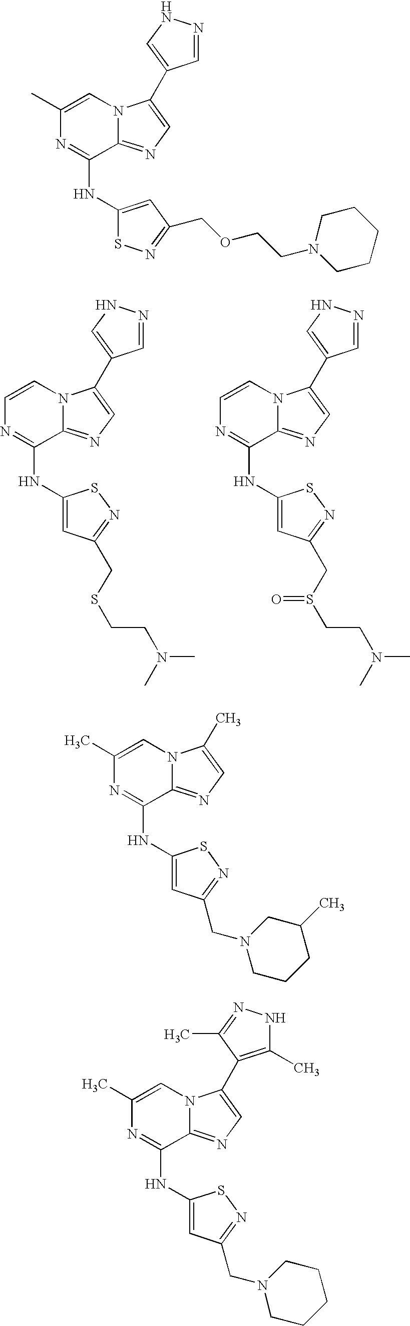 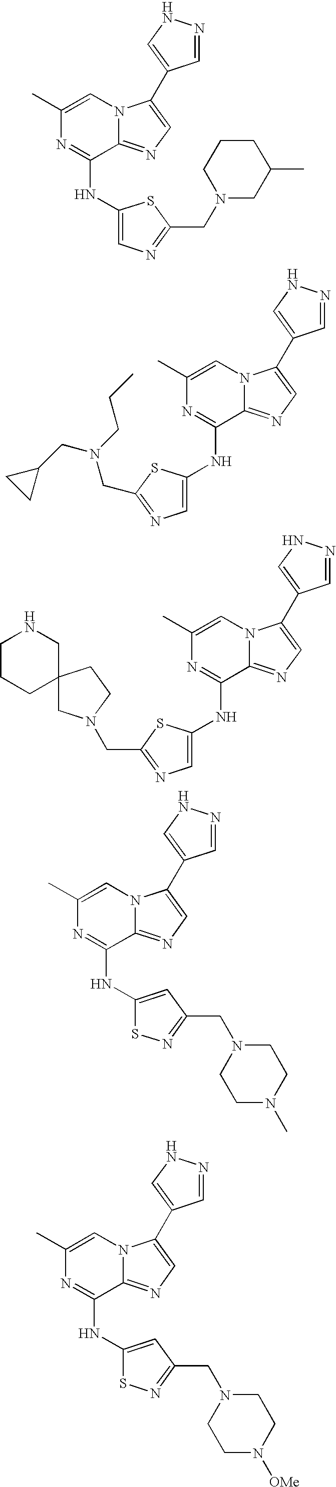 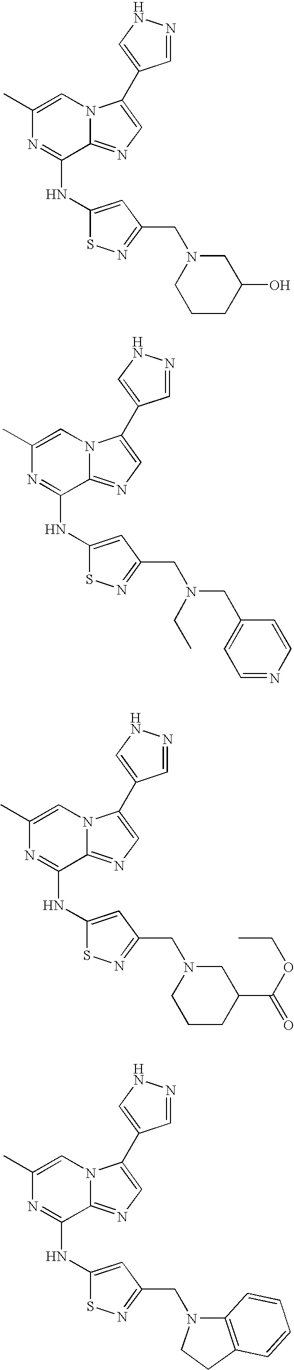 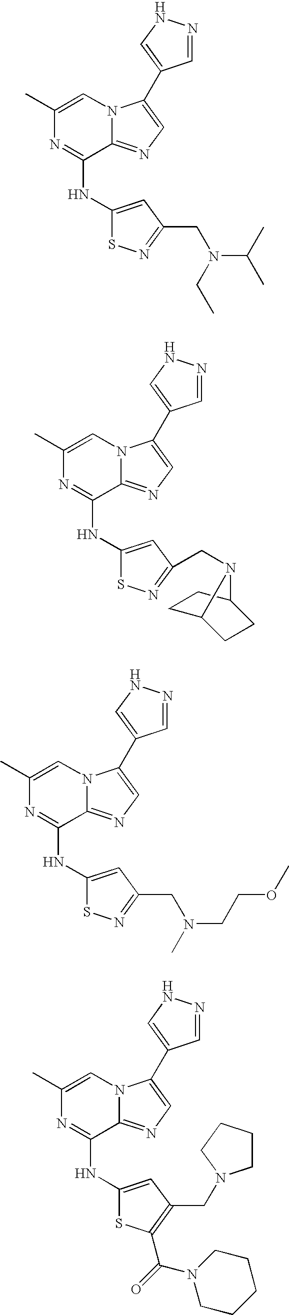 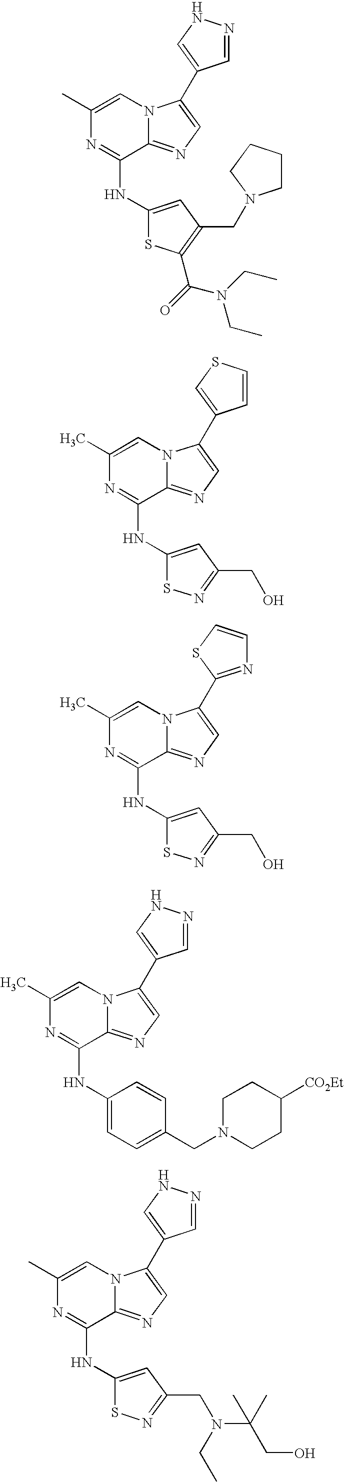 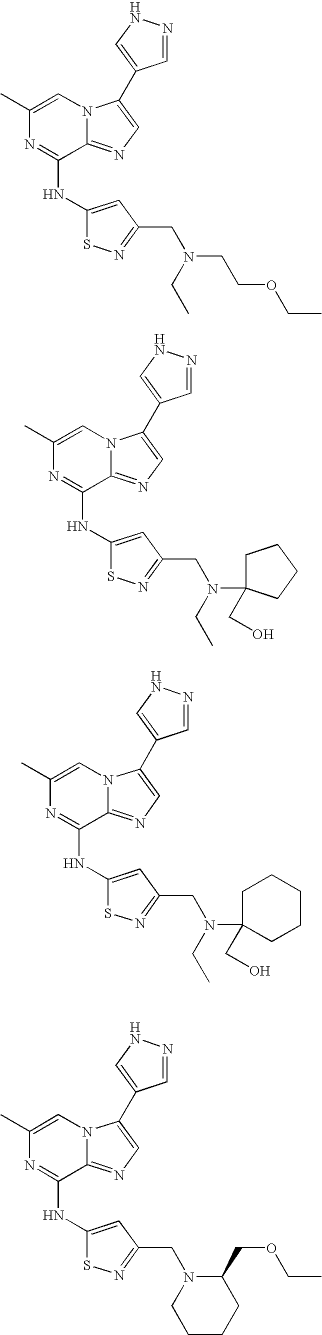 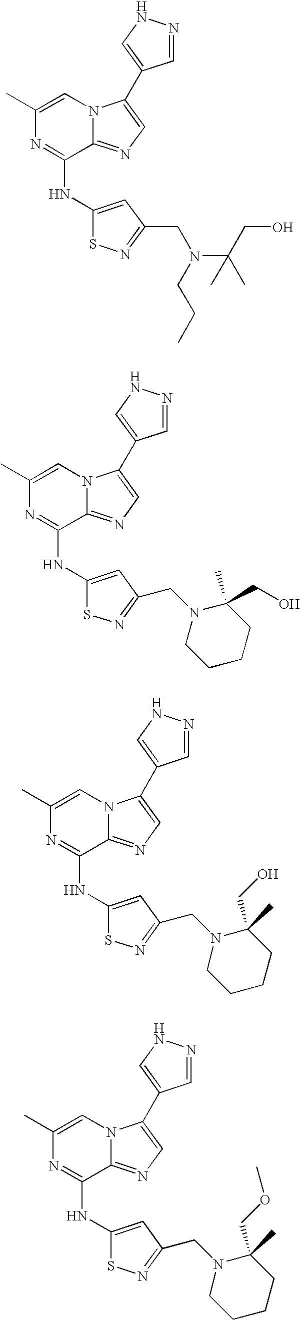 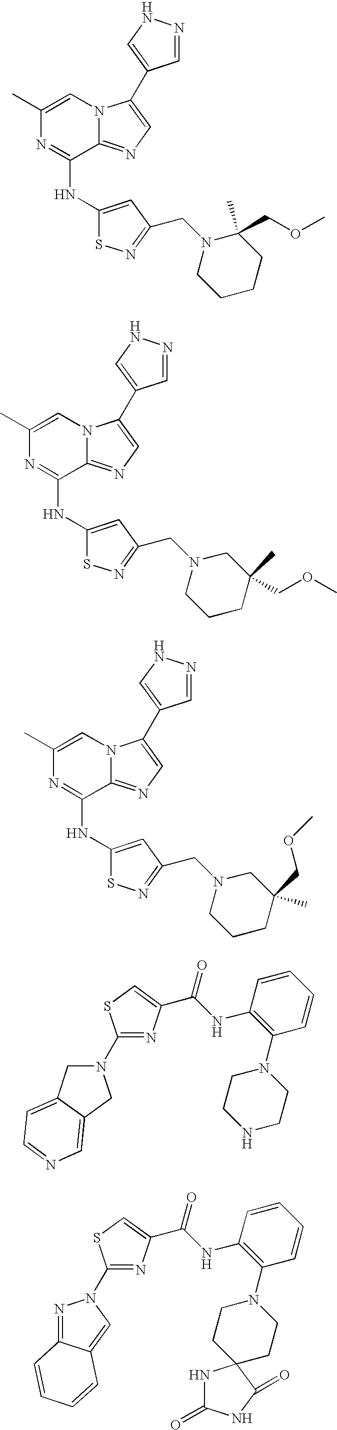 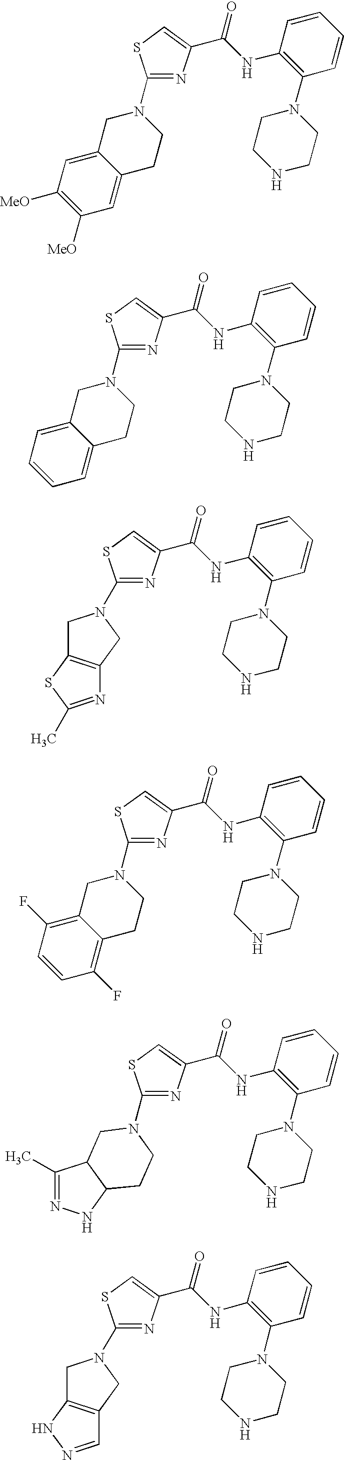 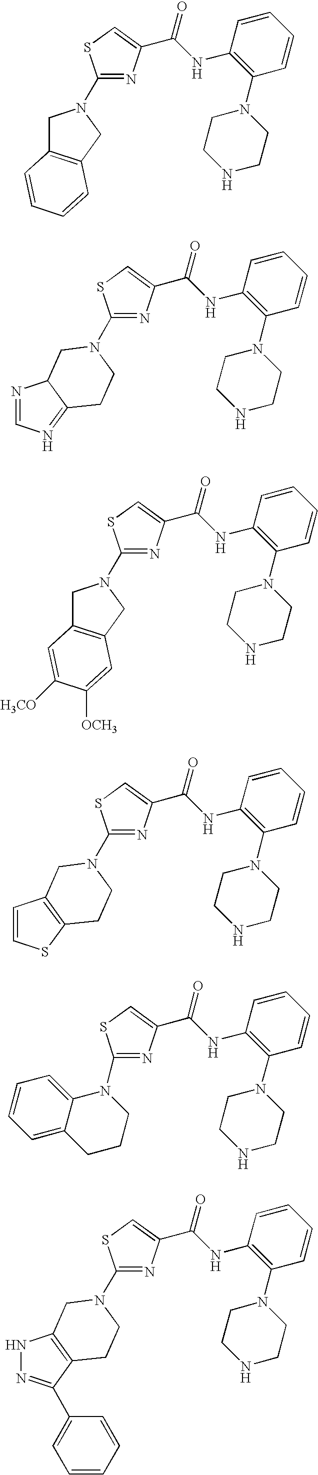 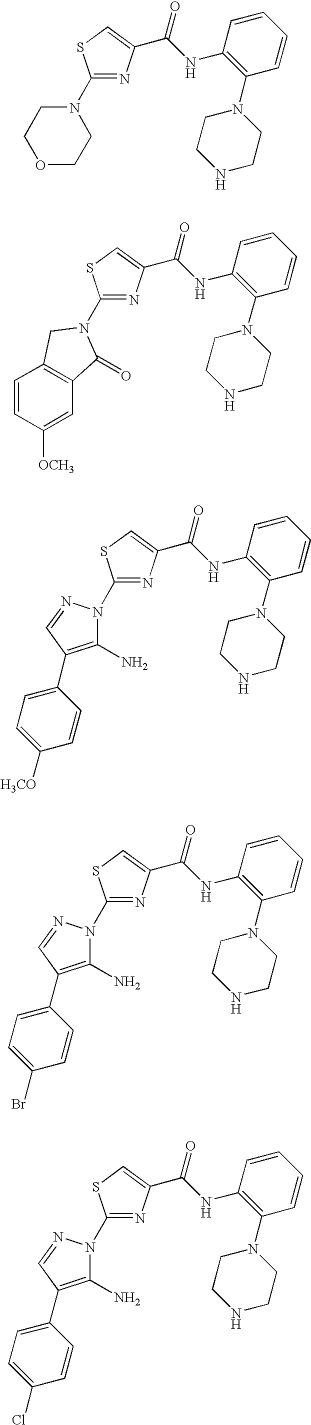 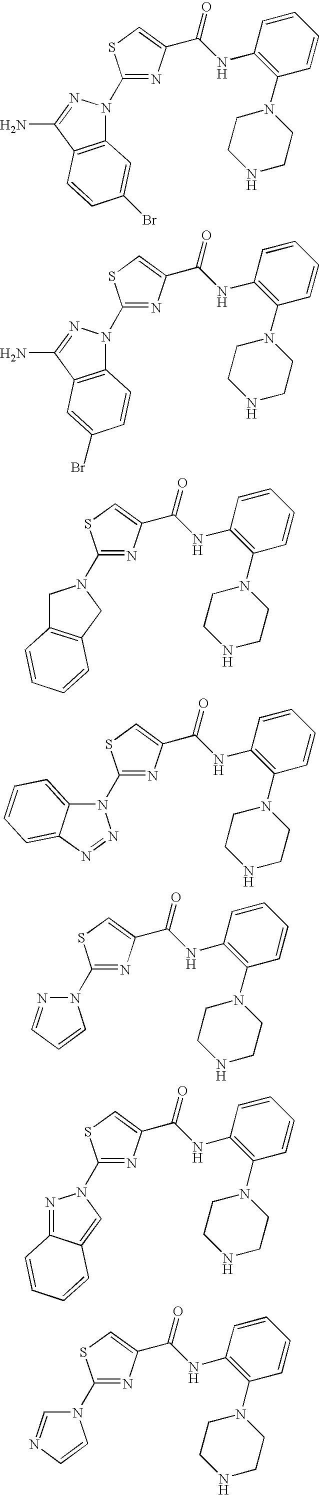 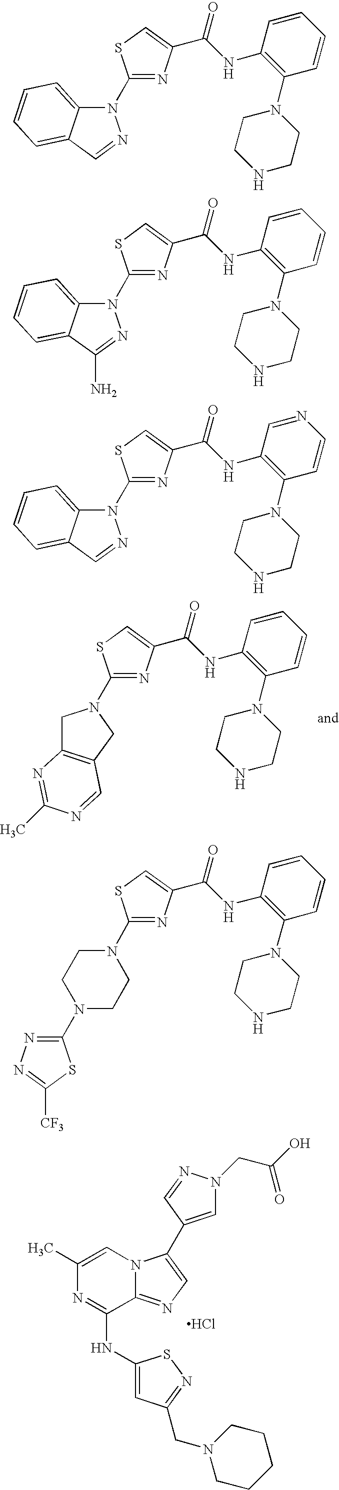 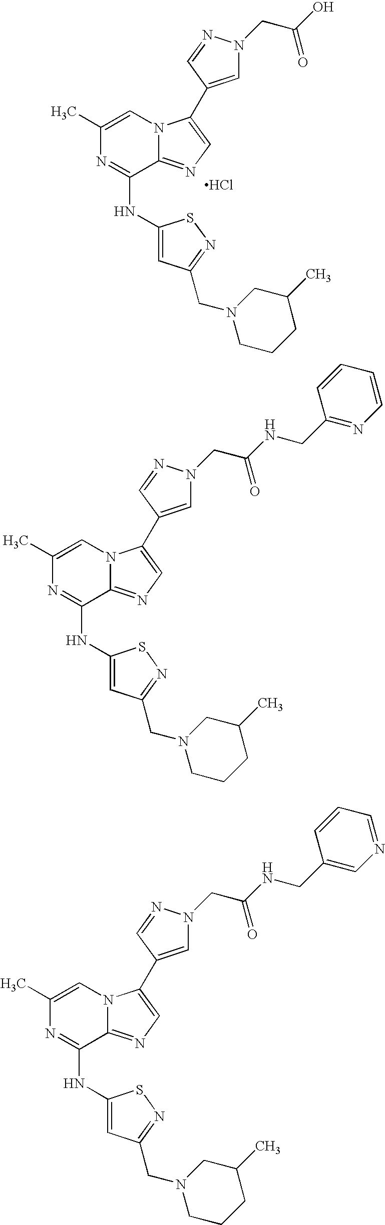 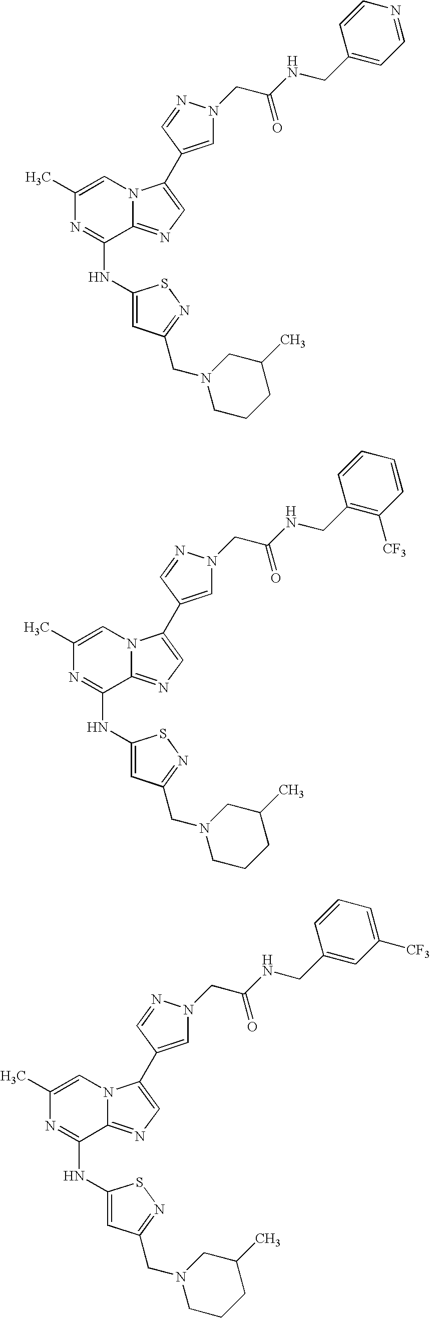 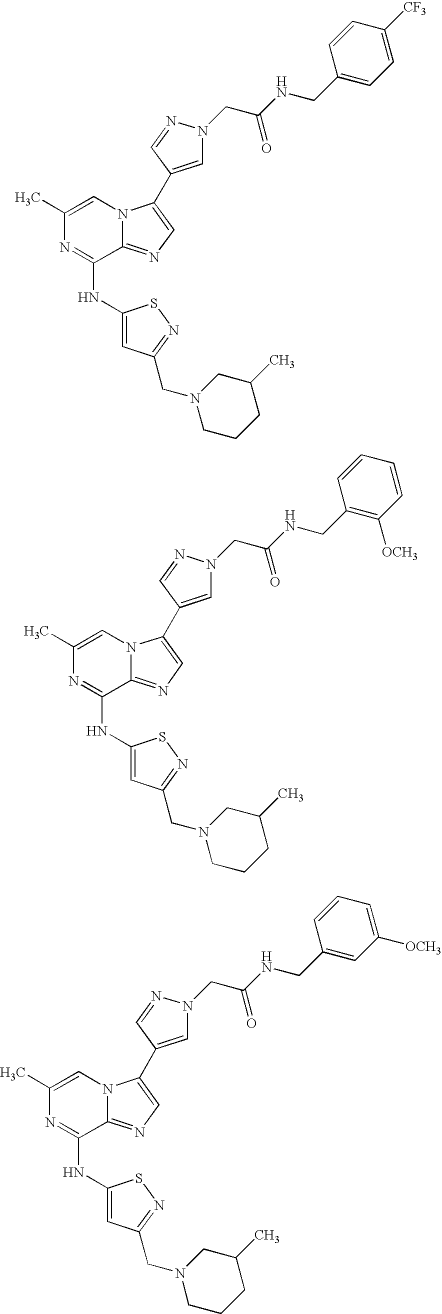 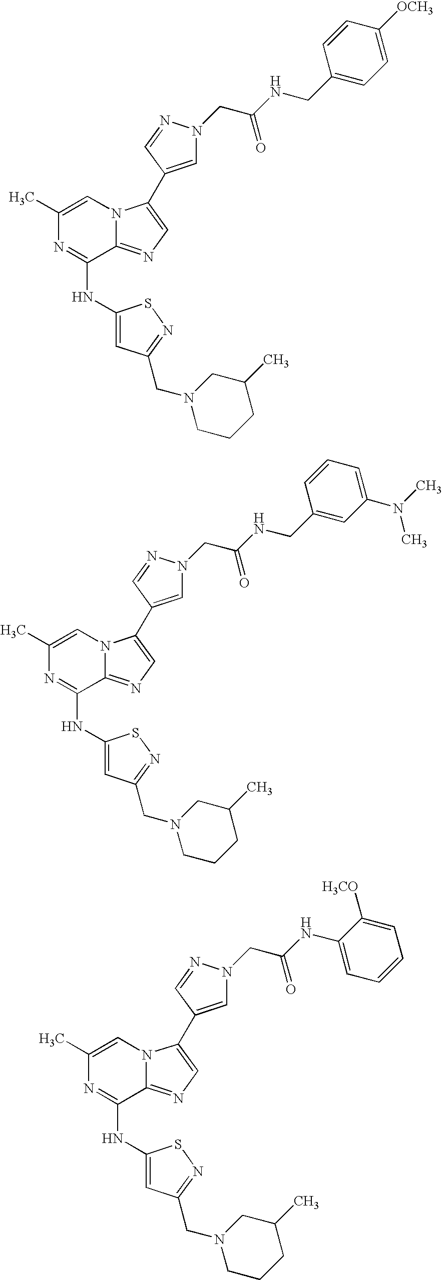 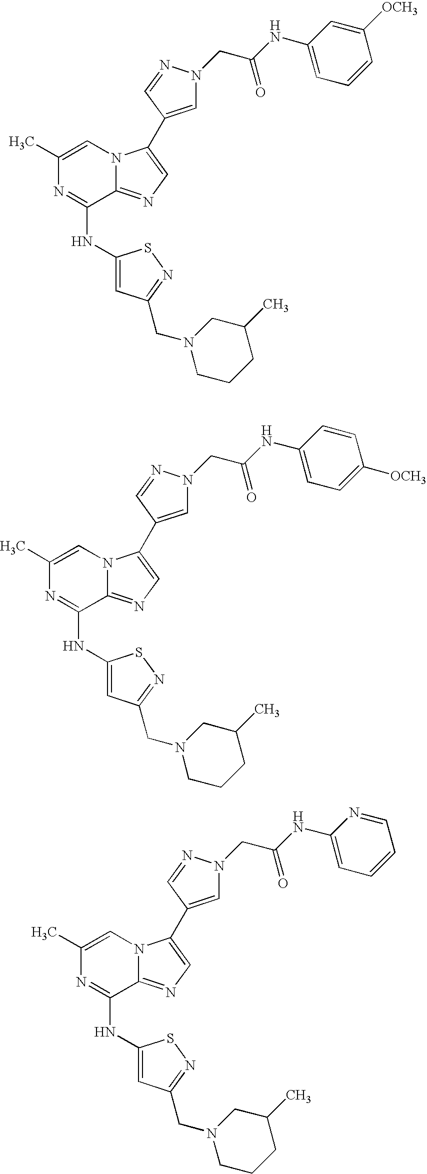 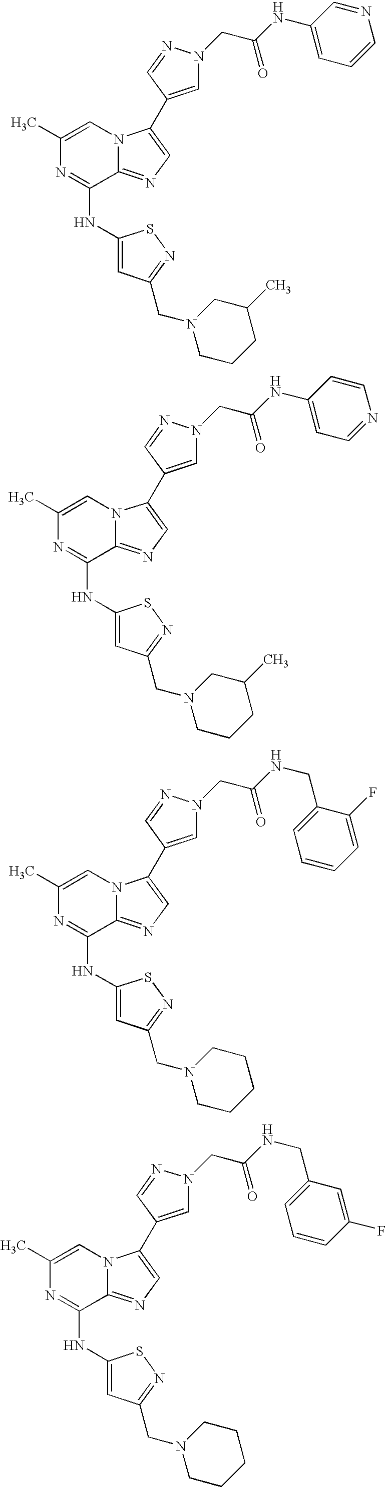 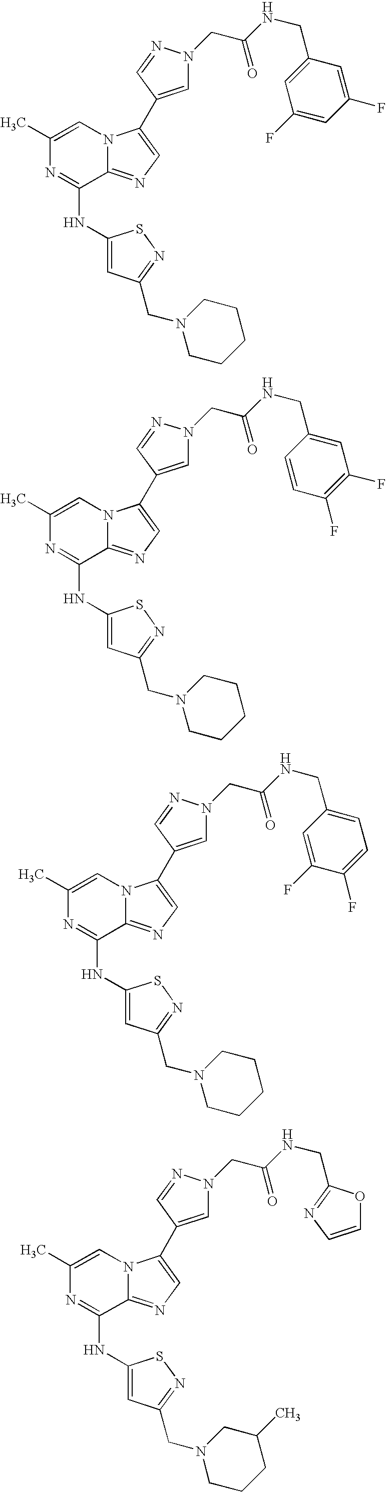 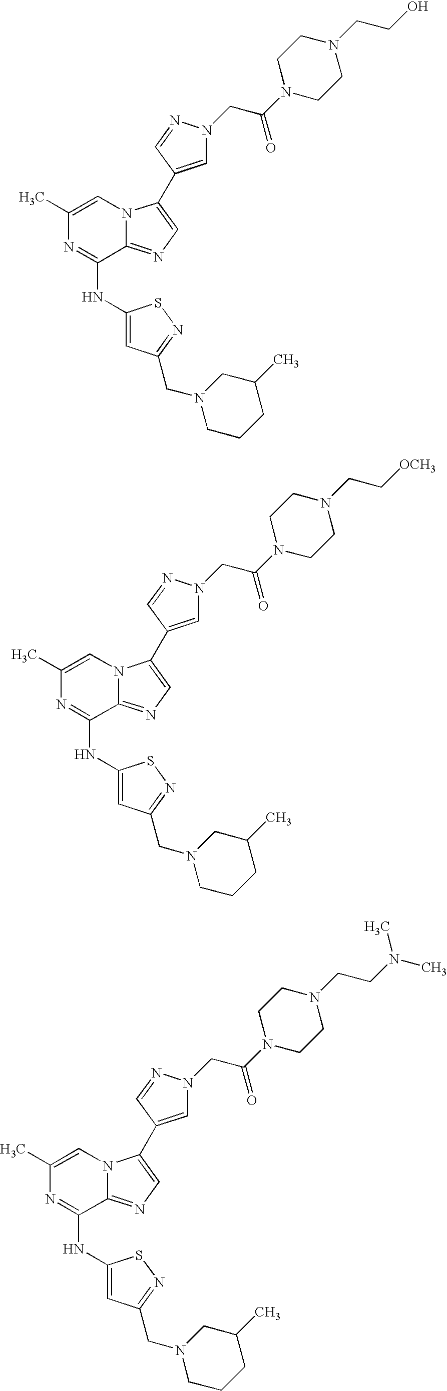 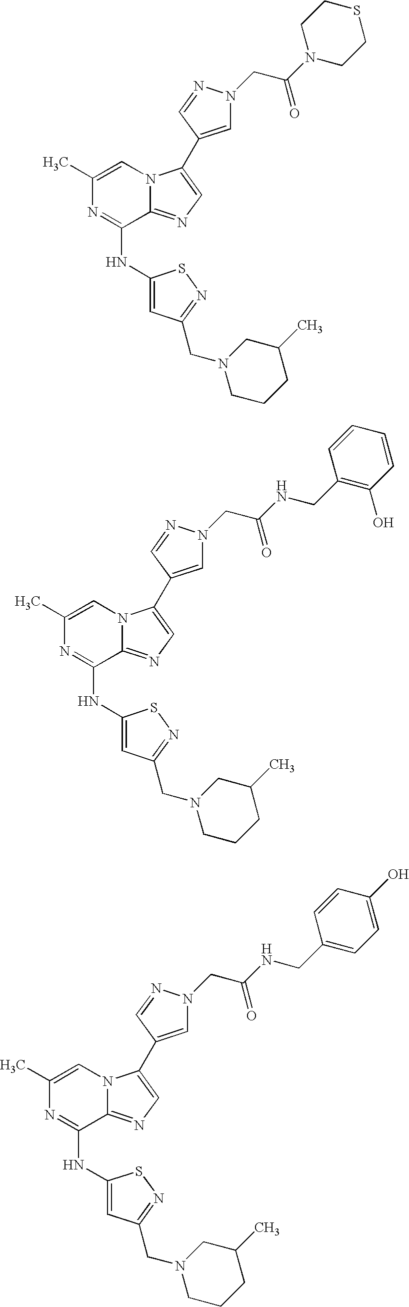 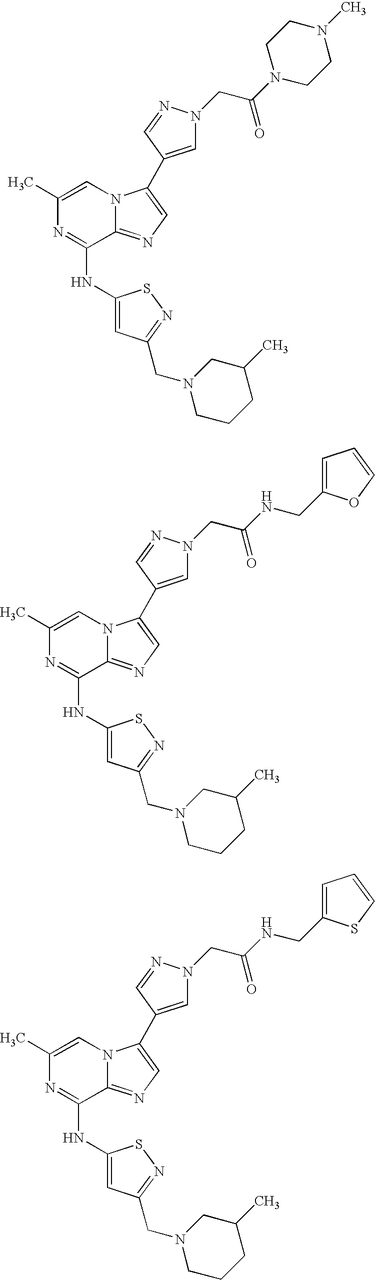 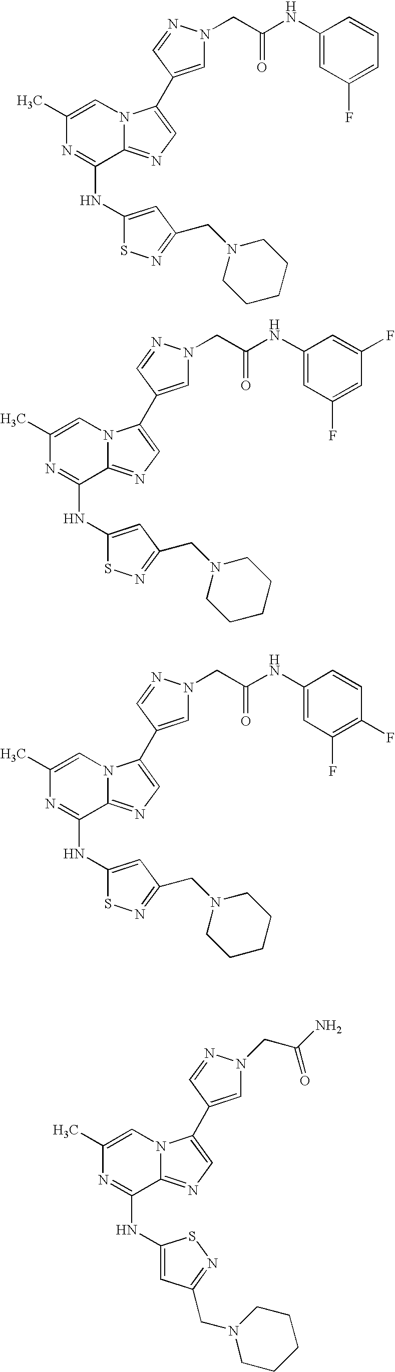 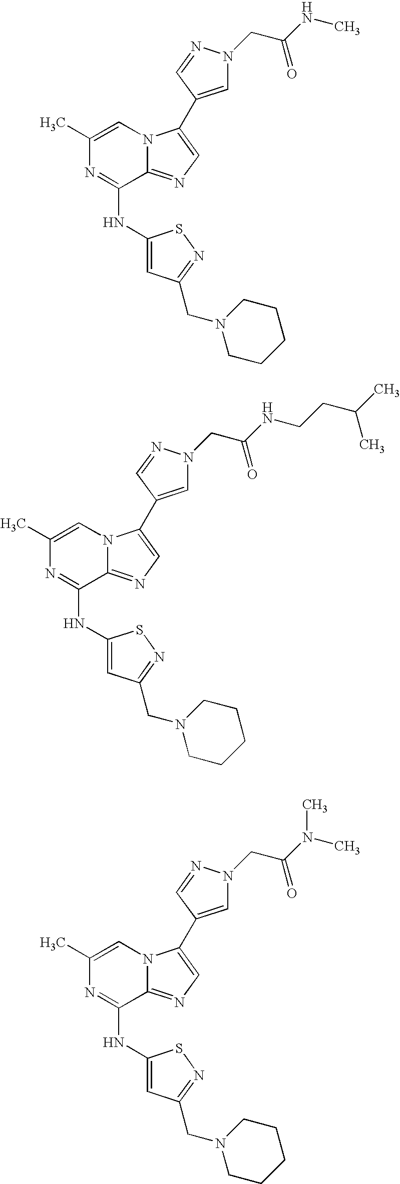 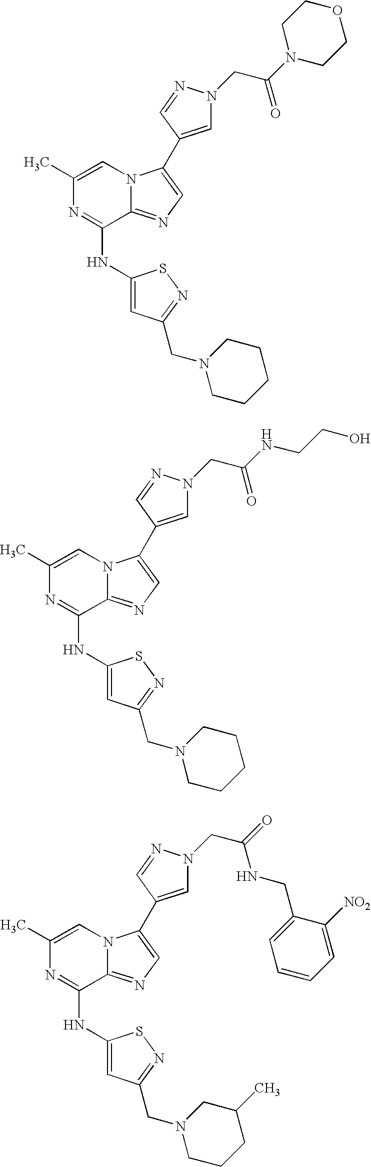 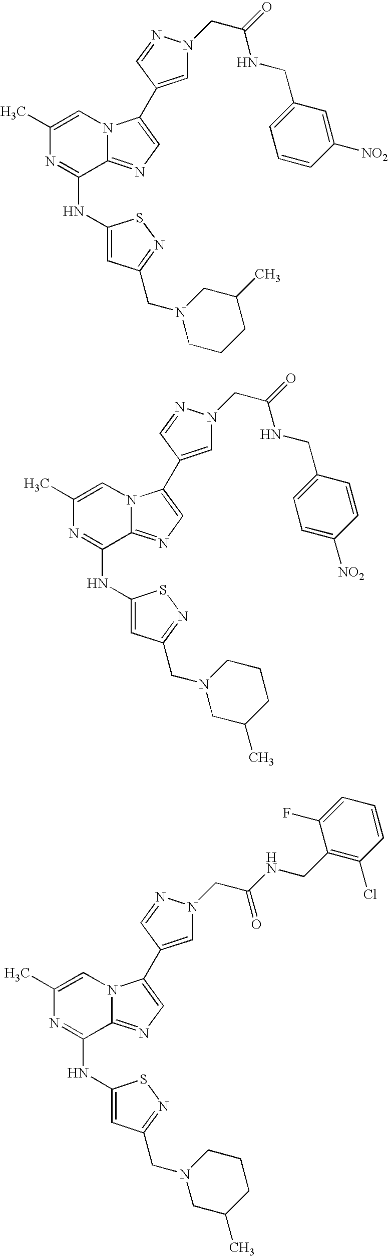 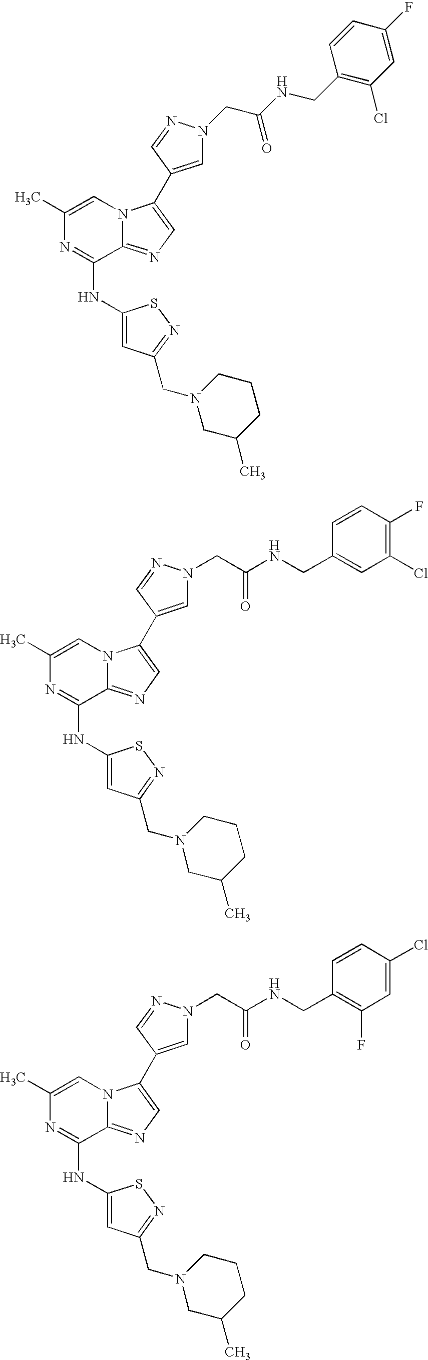 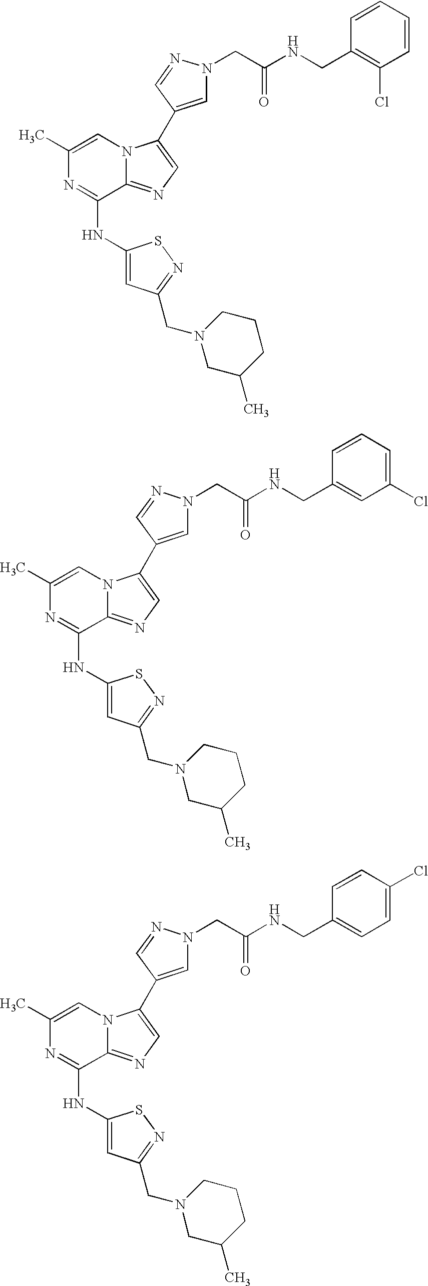 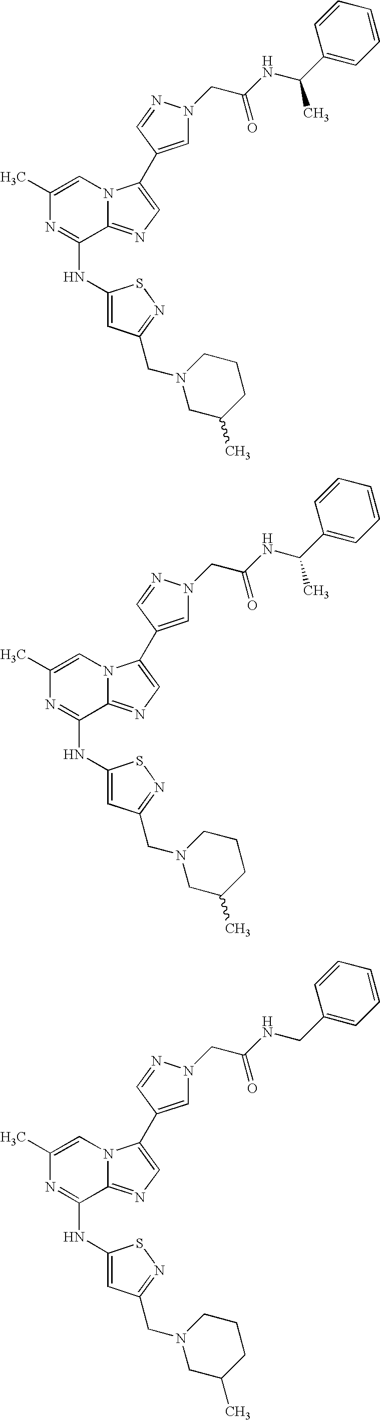 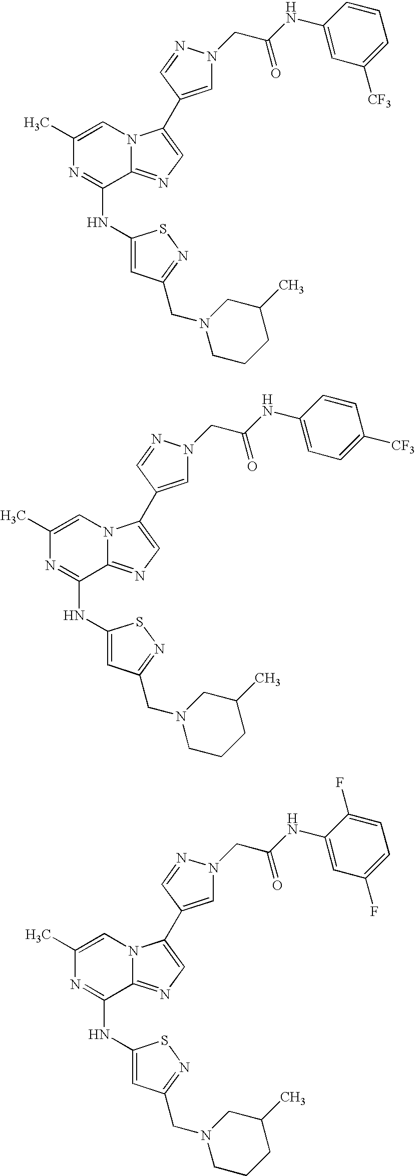 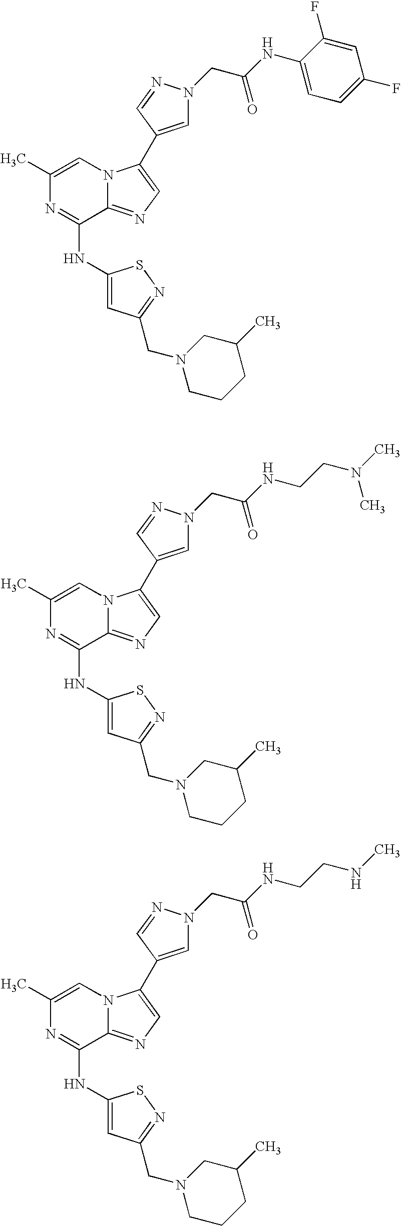 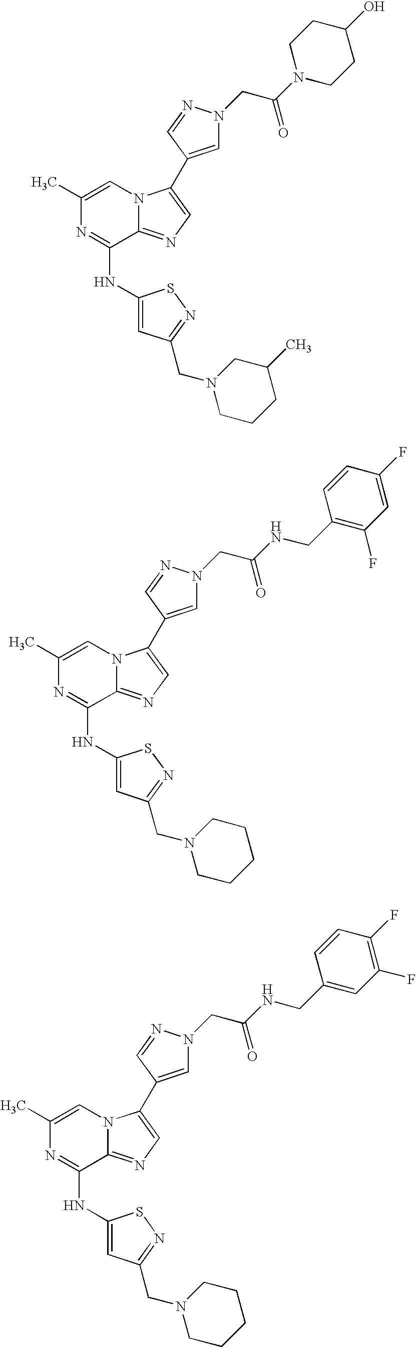 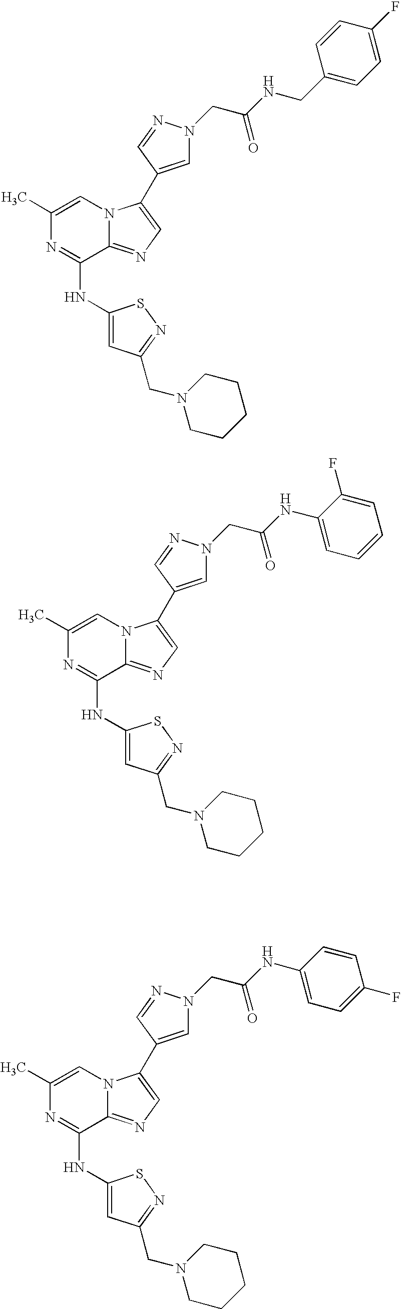 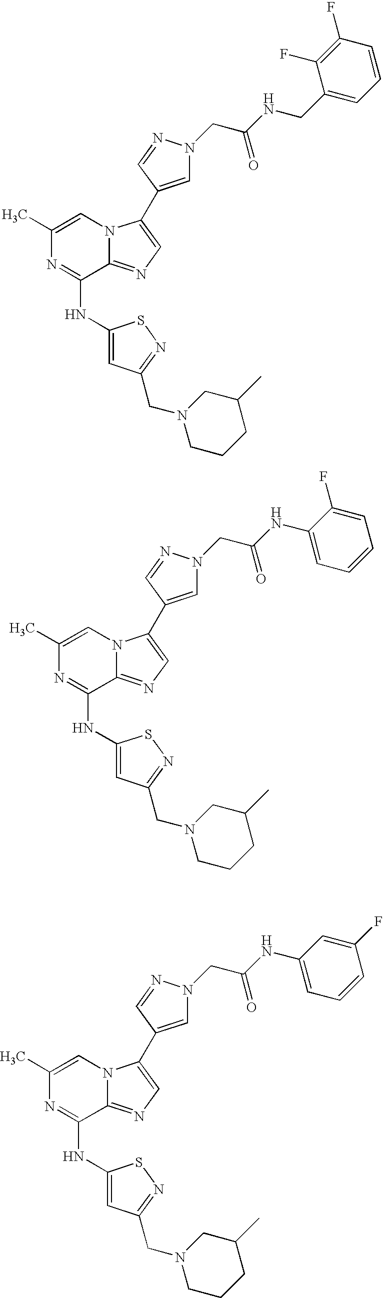 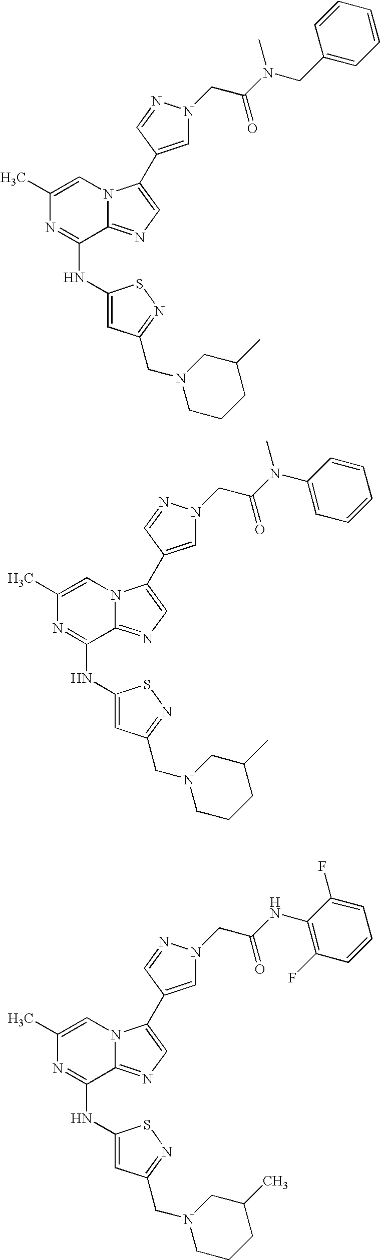 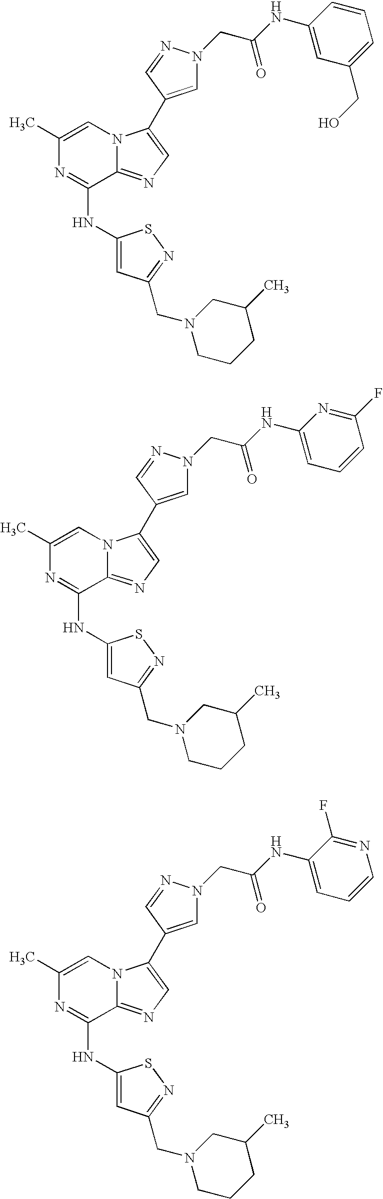 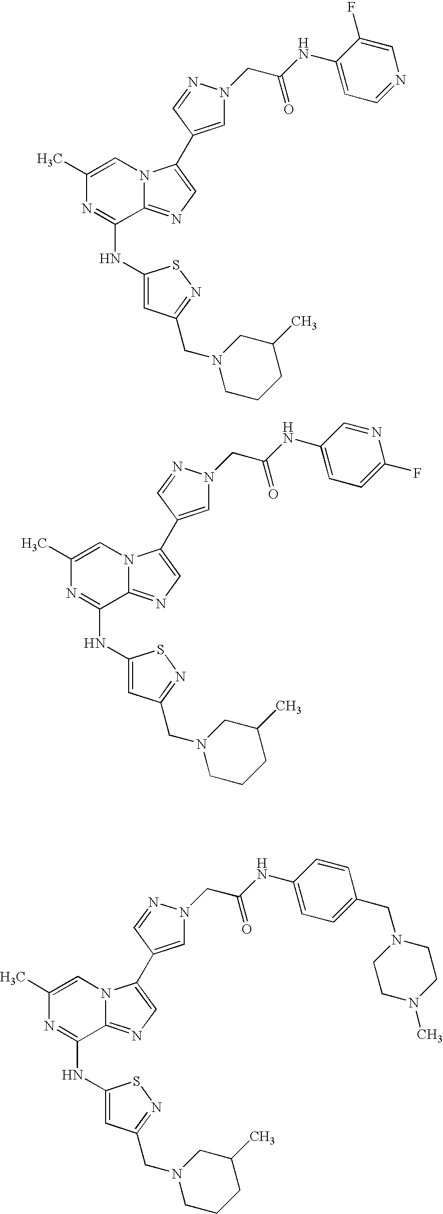 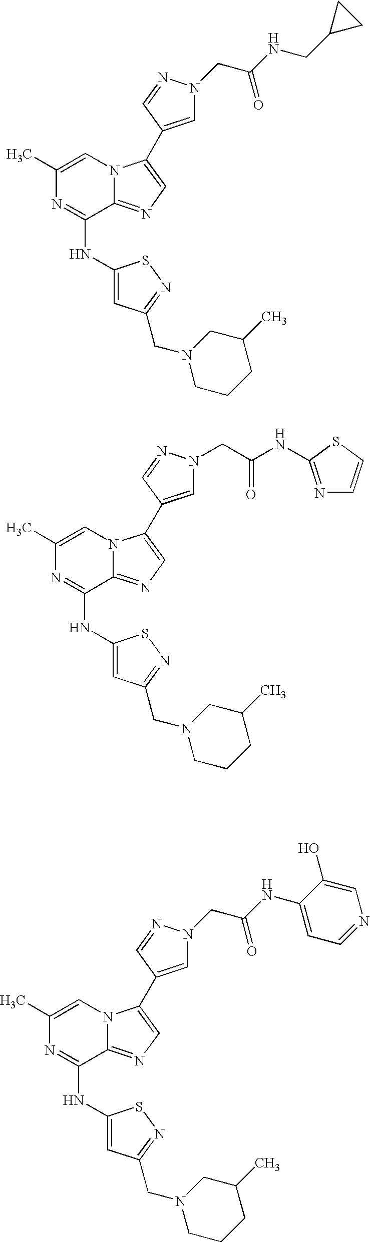 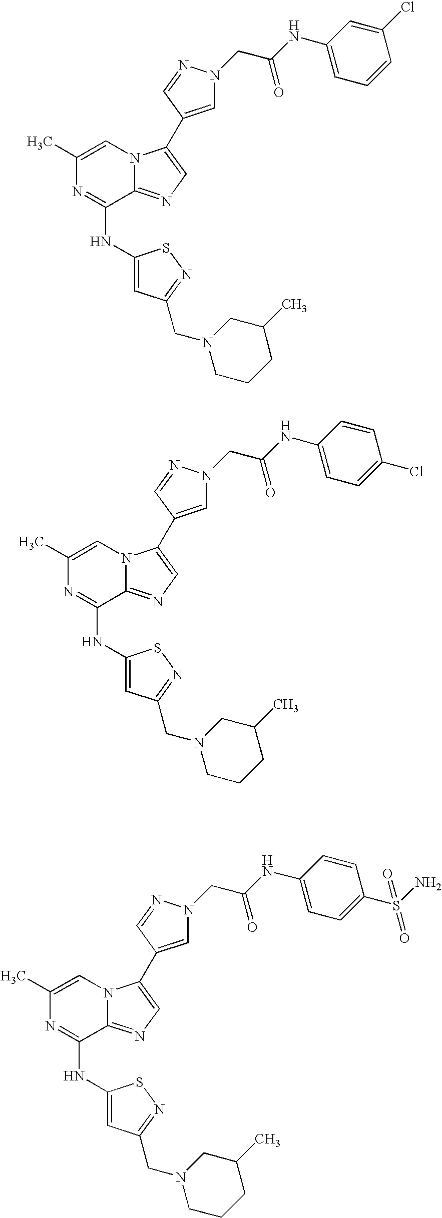 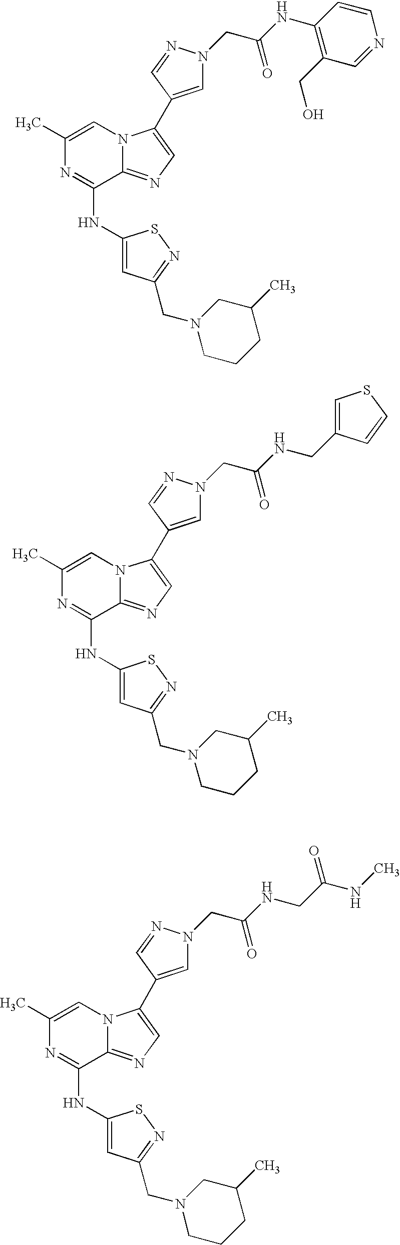 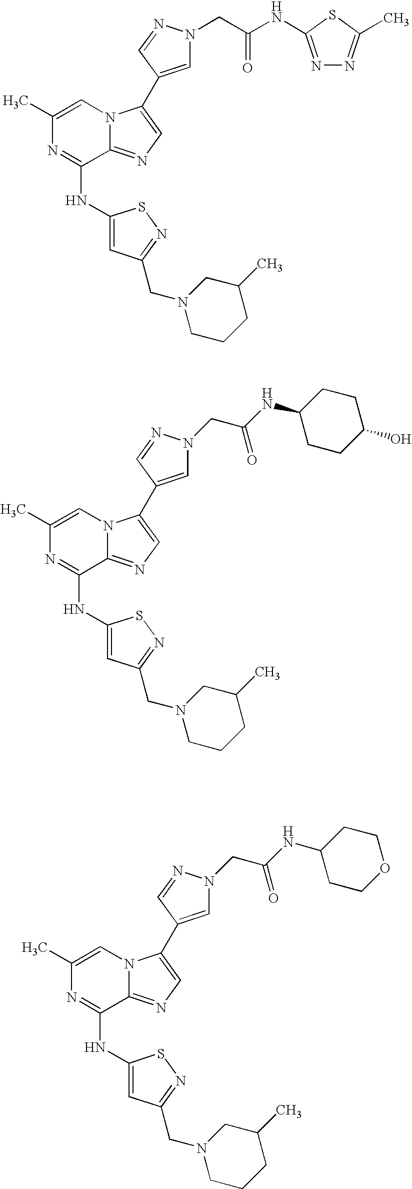 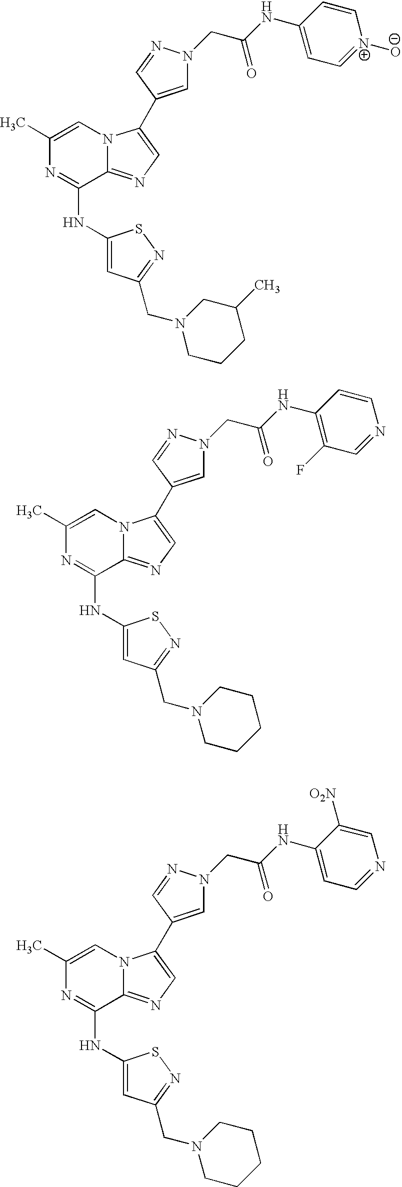 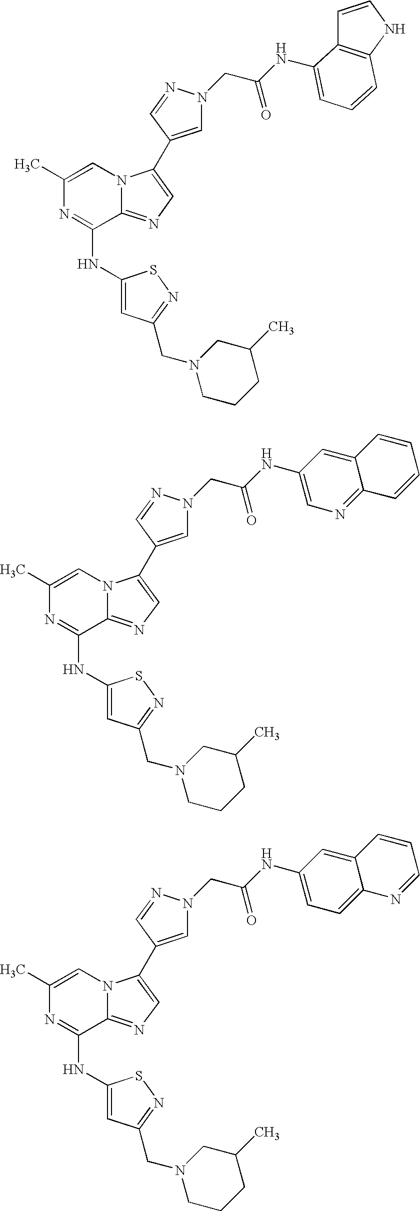 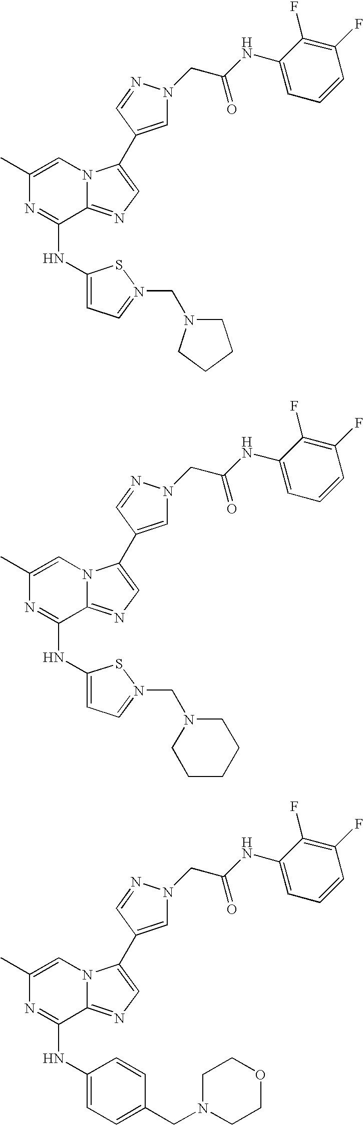 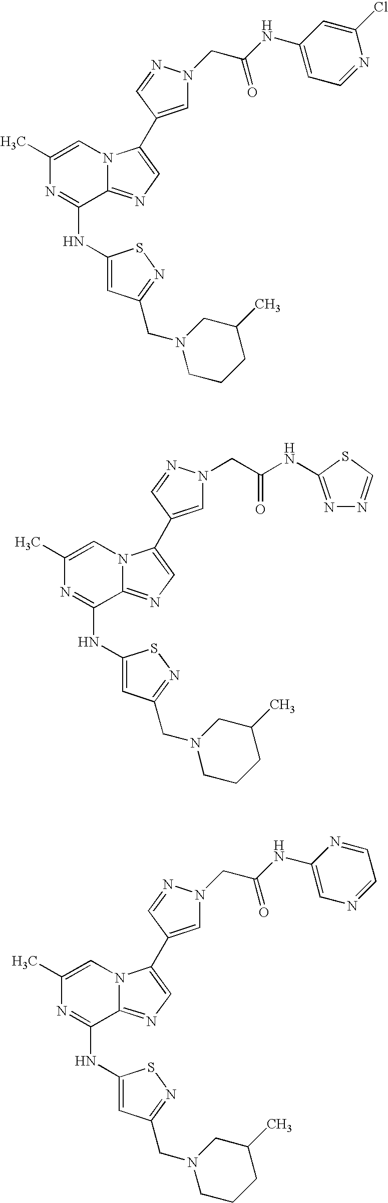 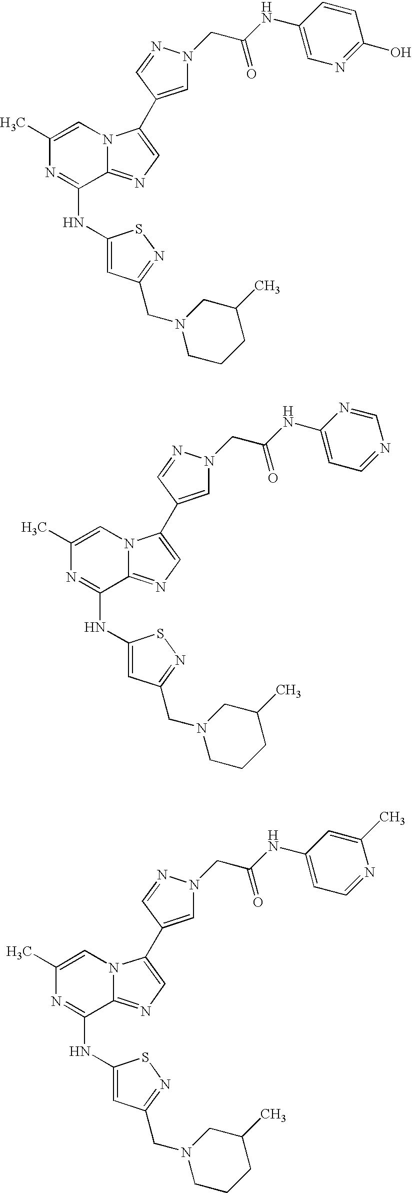 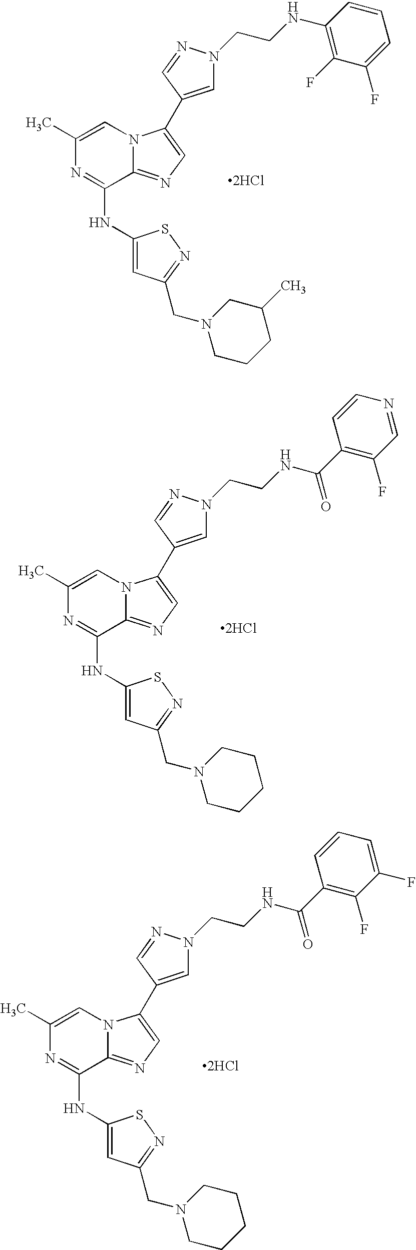 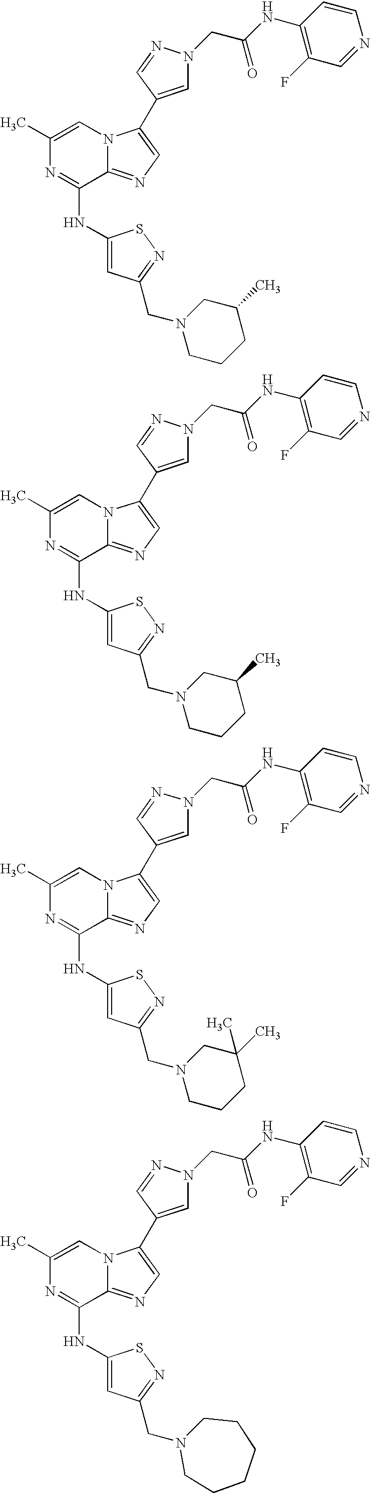 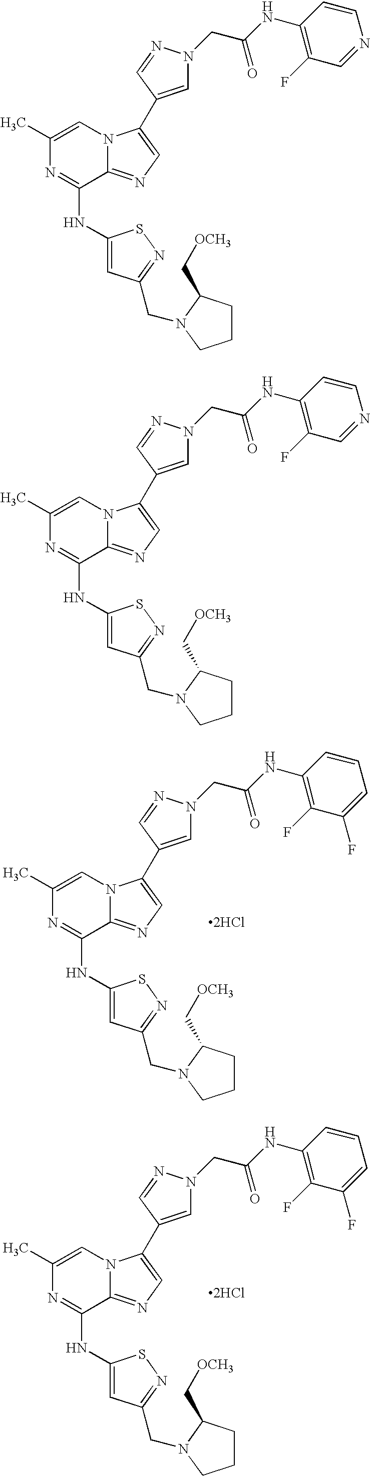 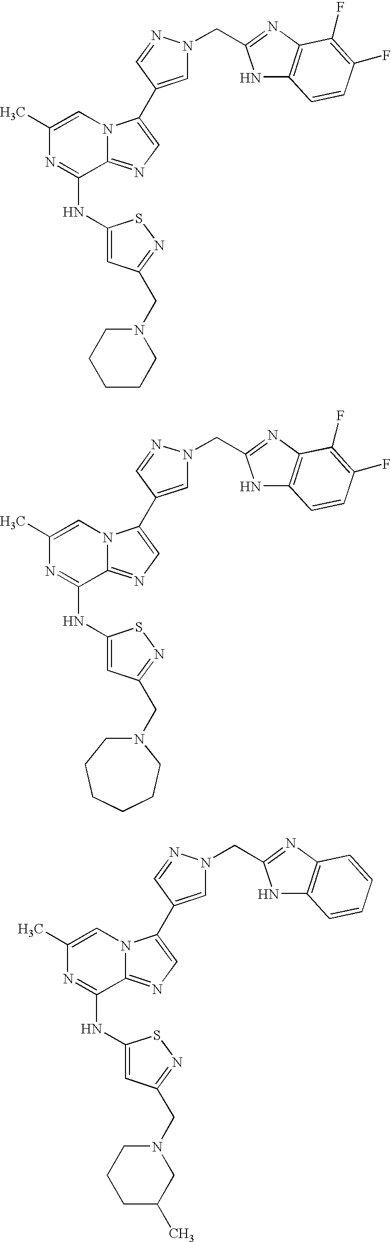 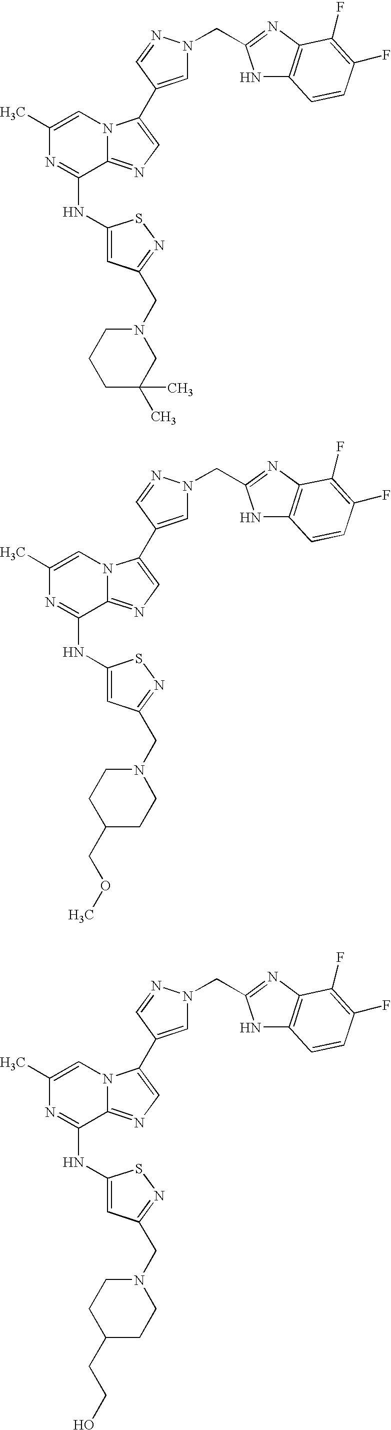 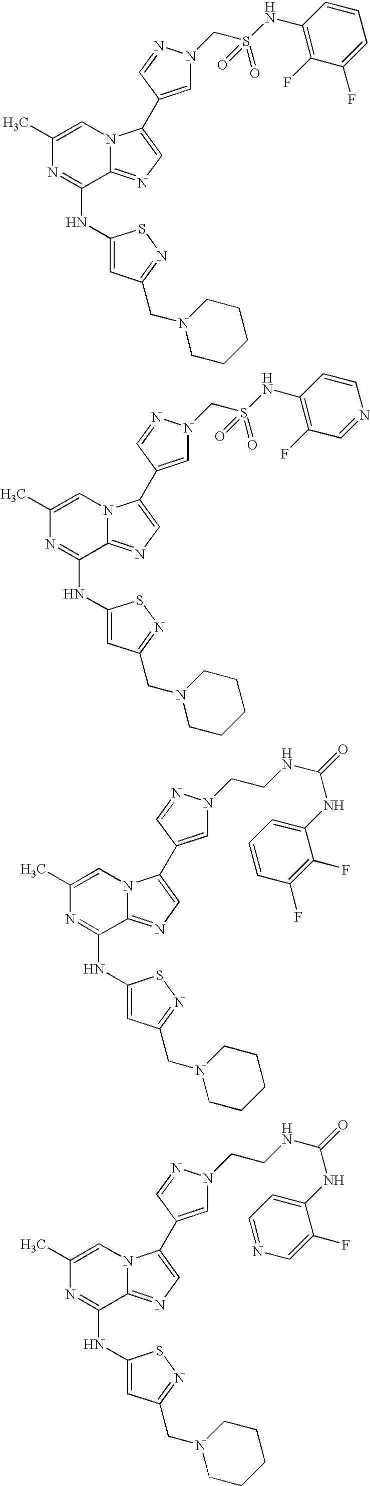 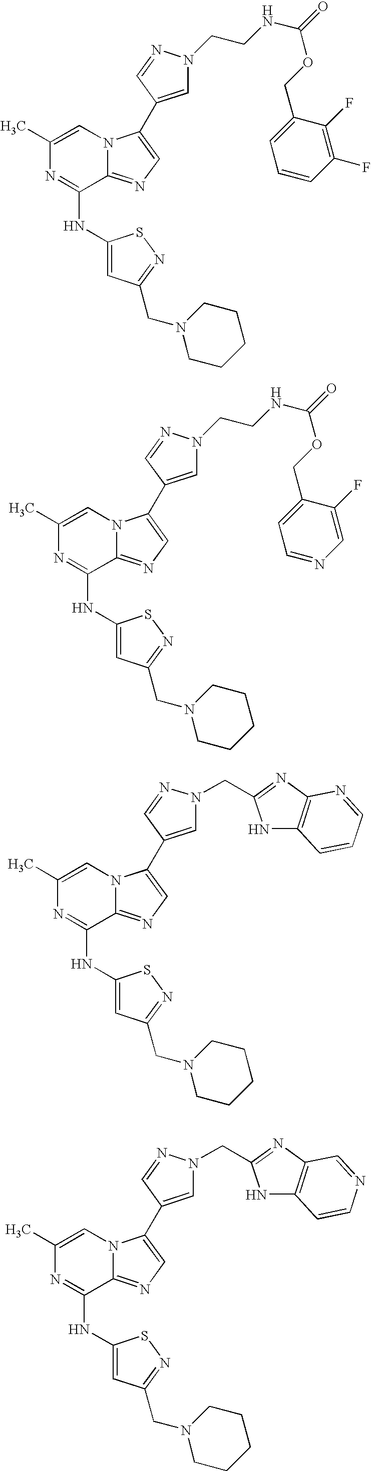 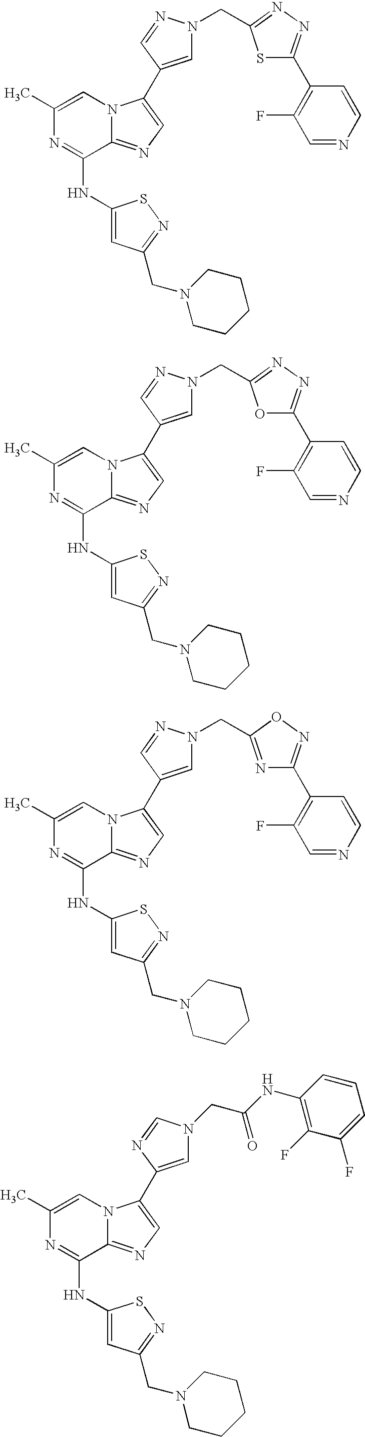 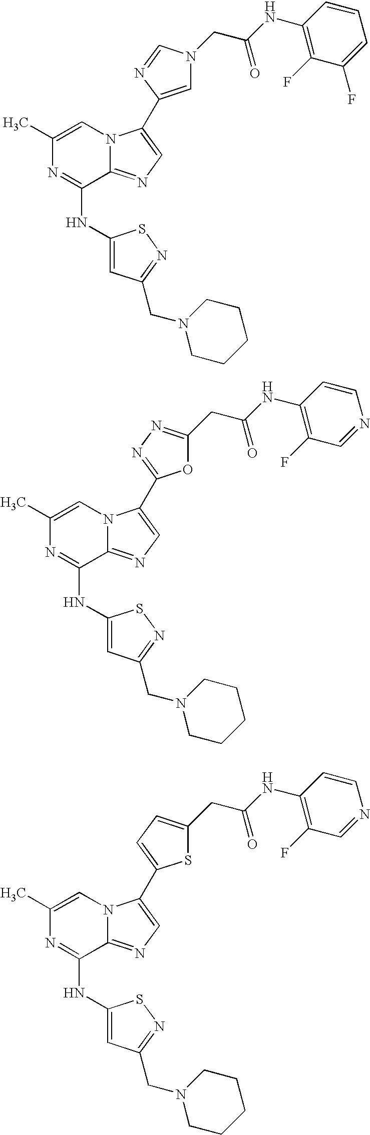 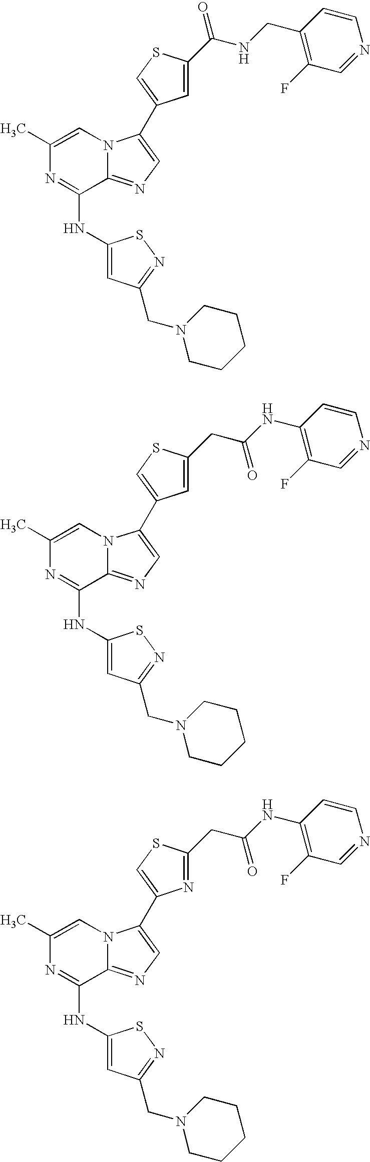 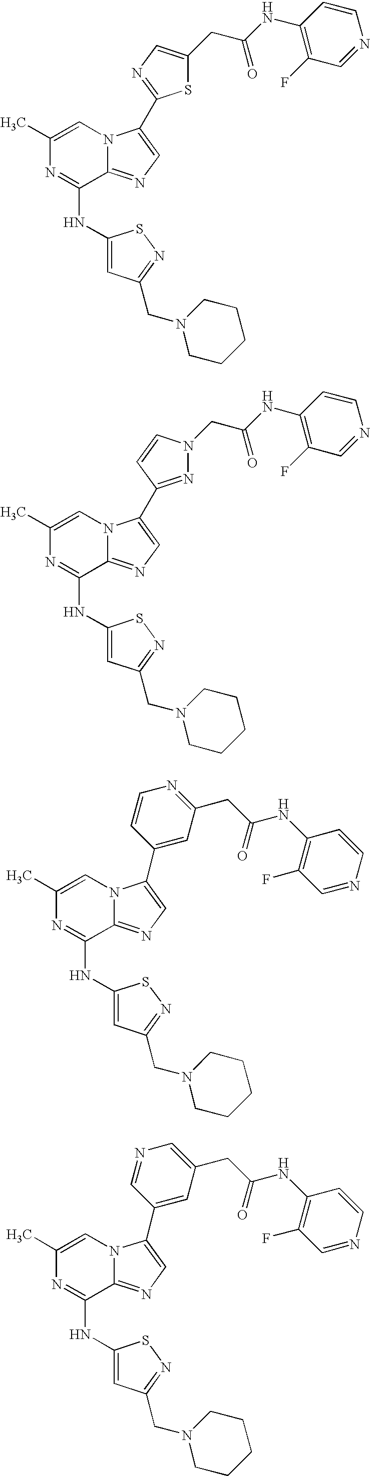 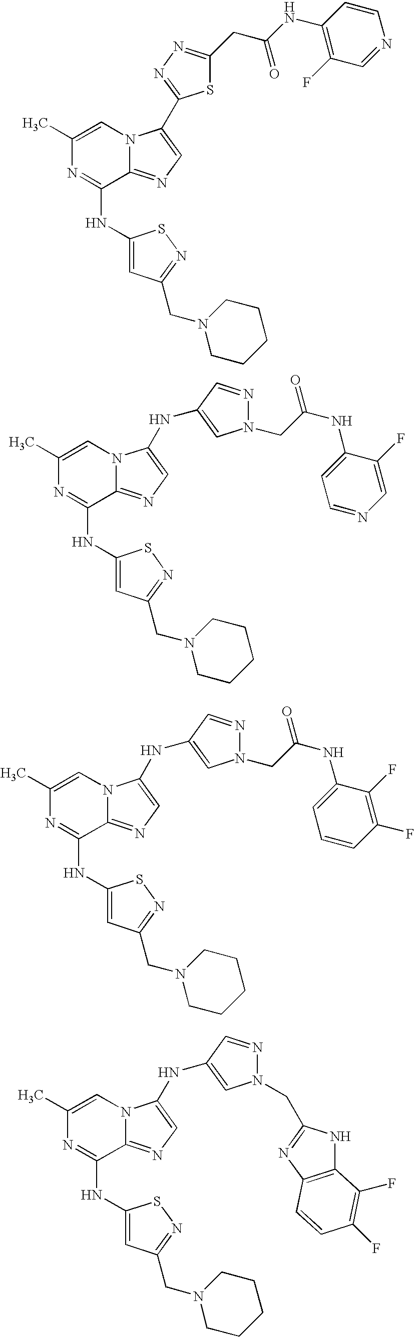 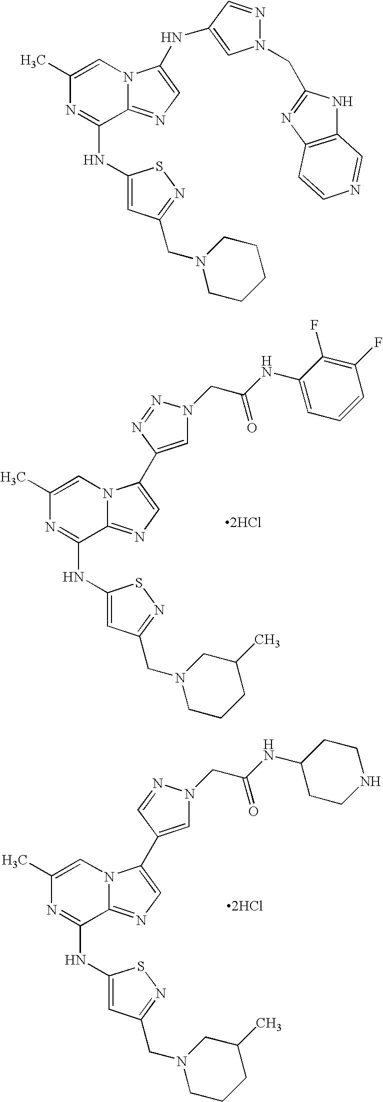 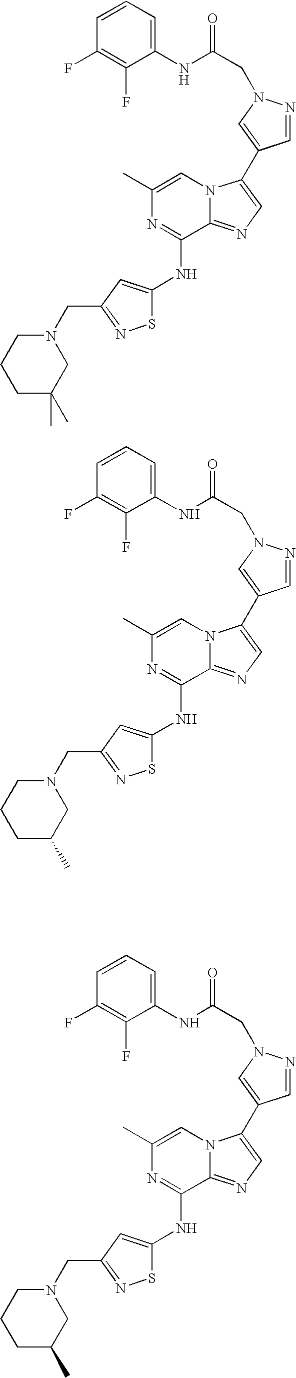
or a pharmaceutically acceptable salt, solvate, ester or prodrug thereof.

15. The method of claim 2, wherein said aurora kinase inhibitors represented by Formulas E-K are selected from the group consisting of: 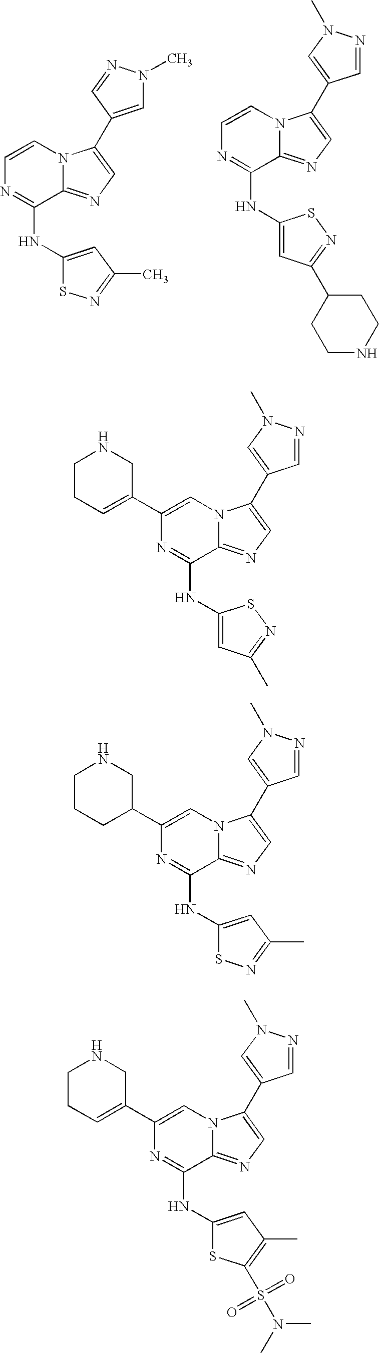 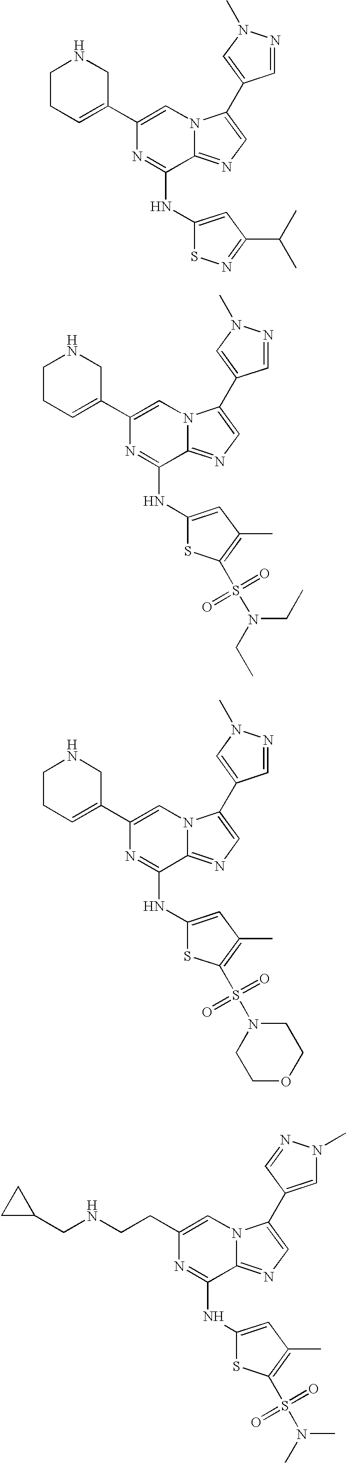 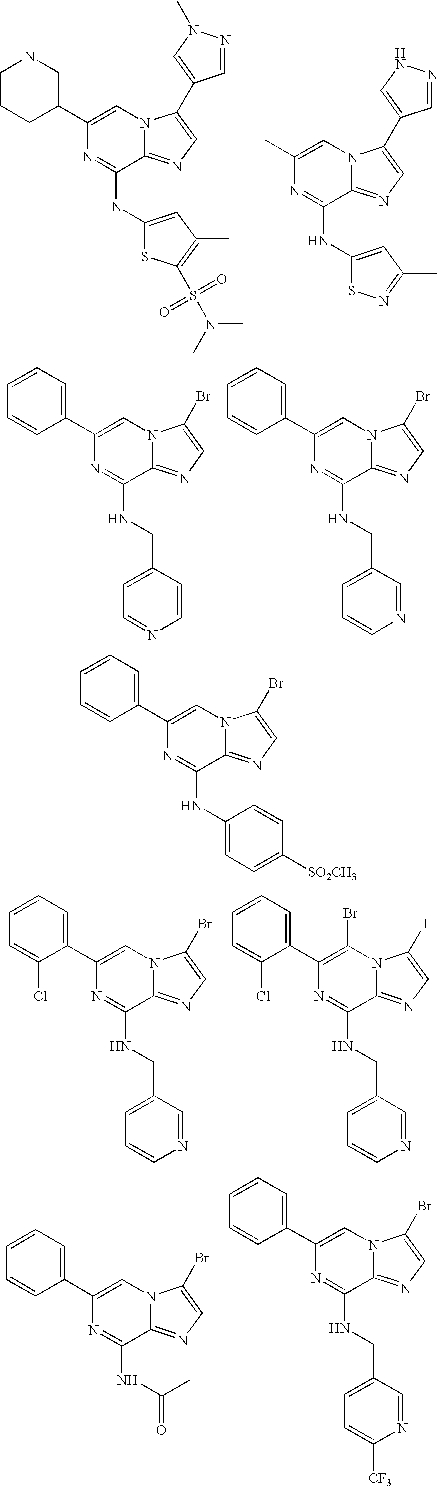 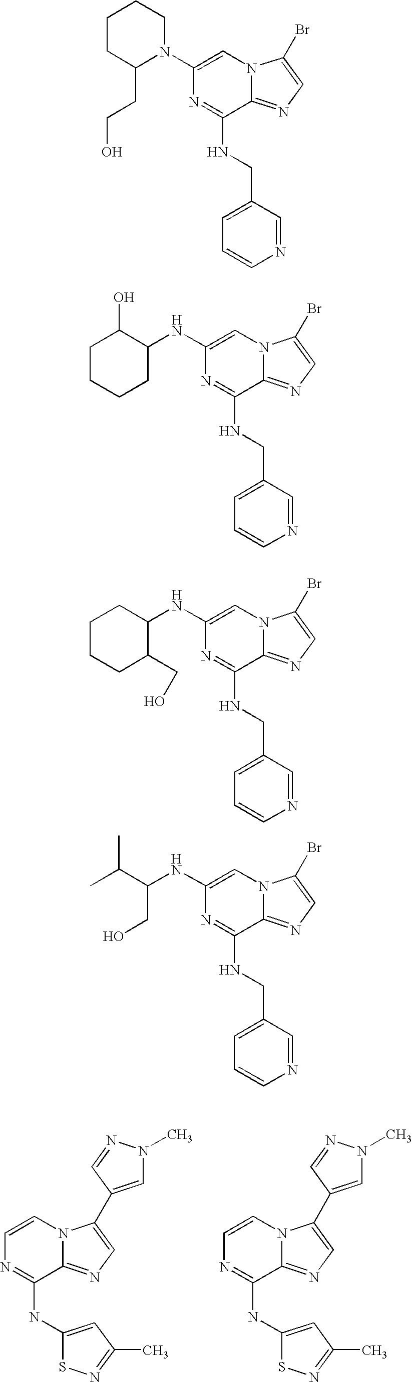 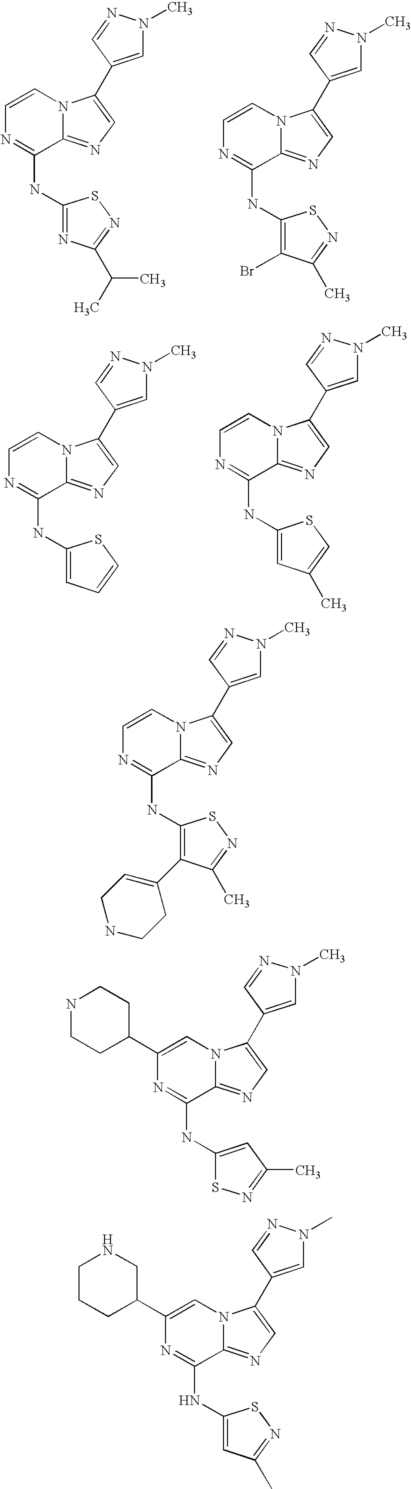 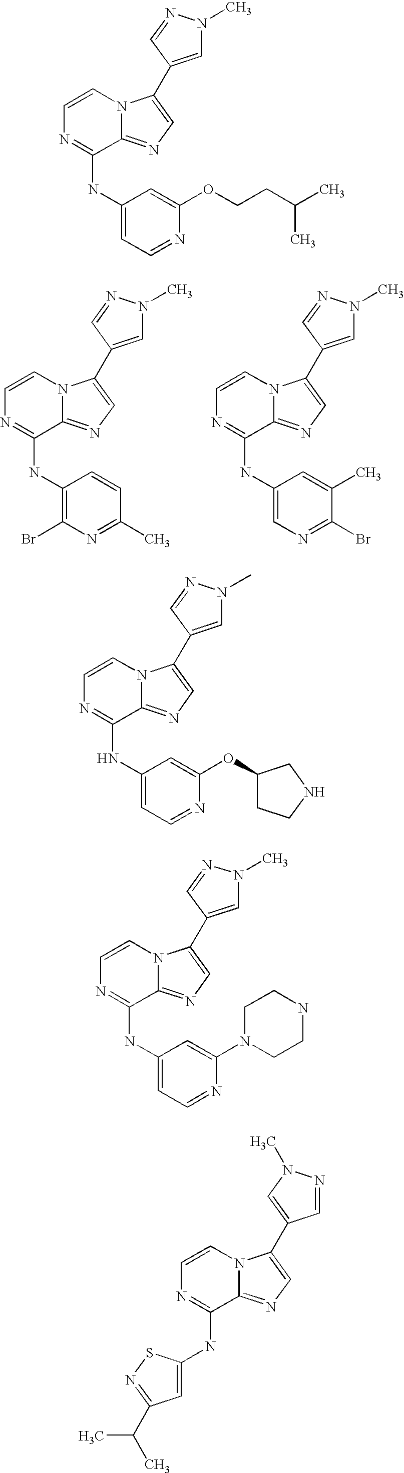 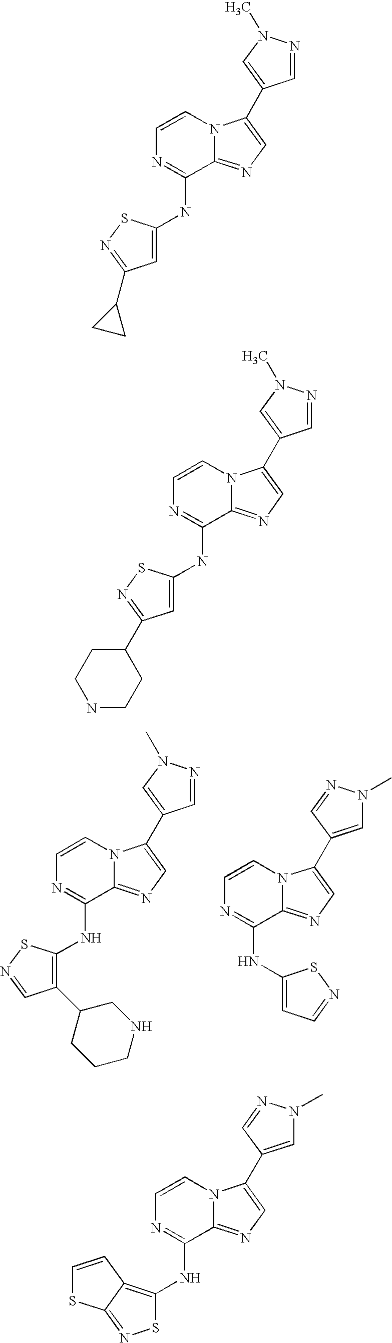 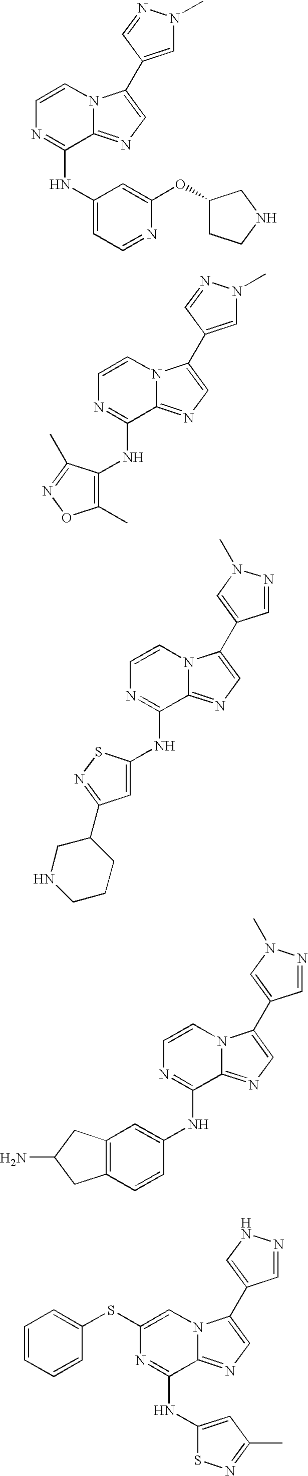 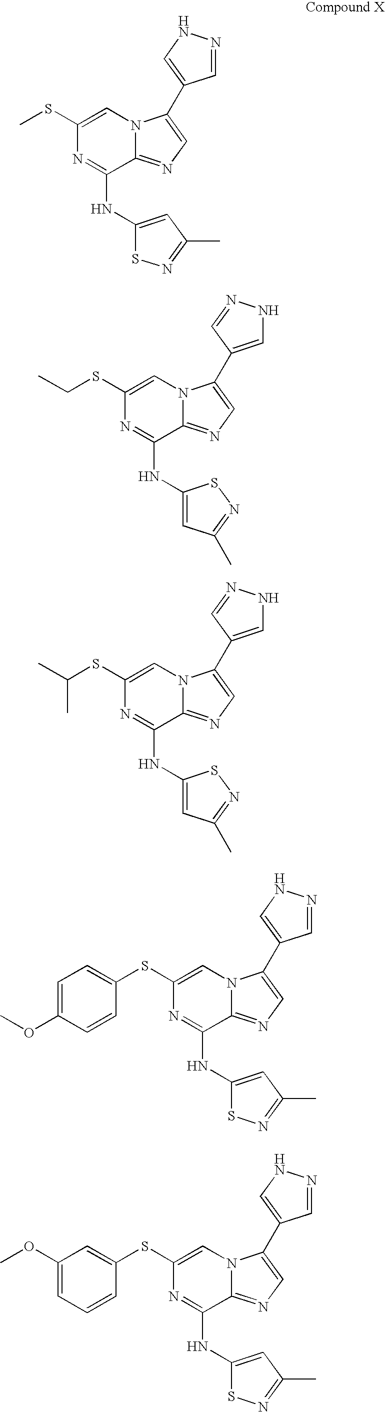 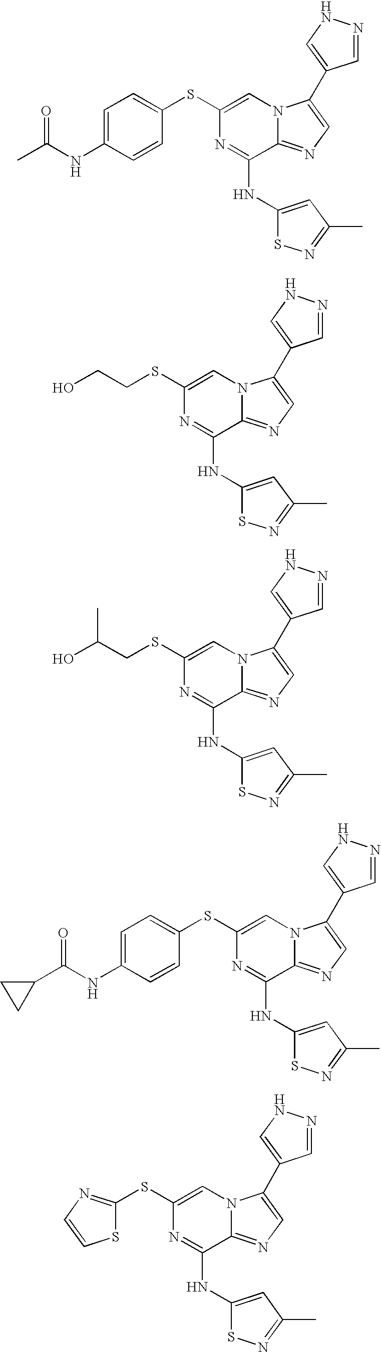 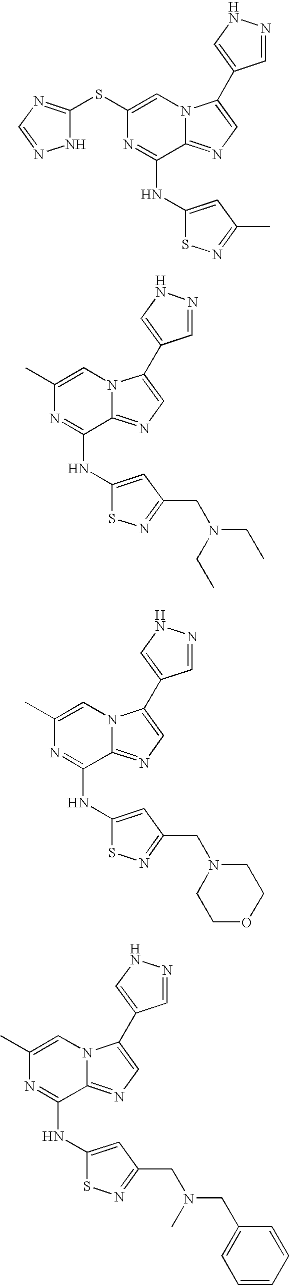 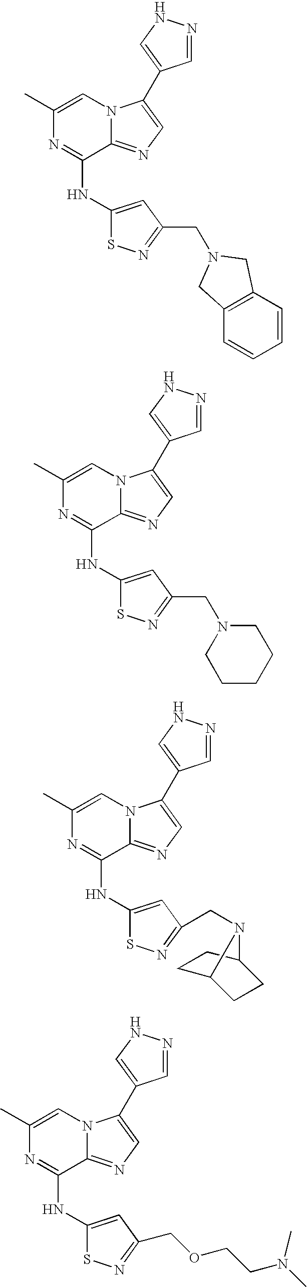 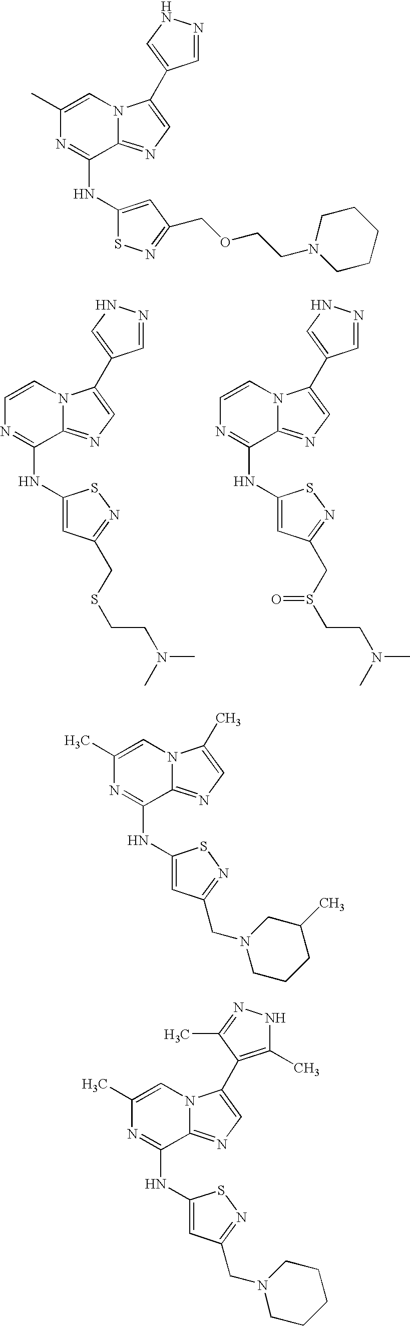 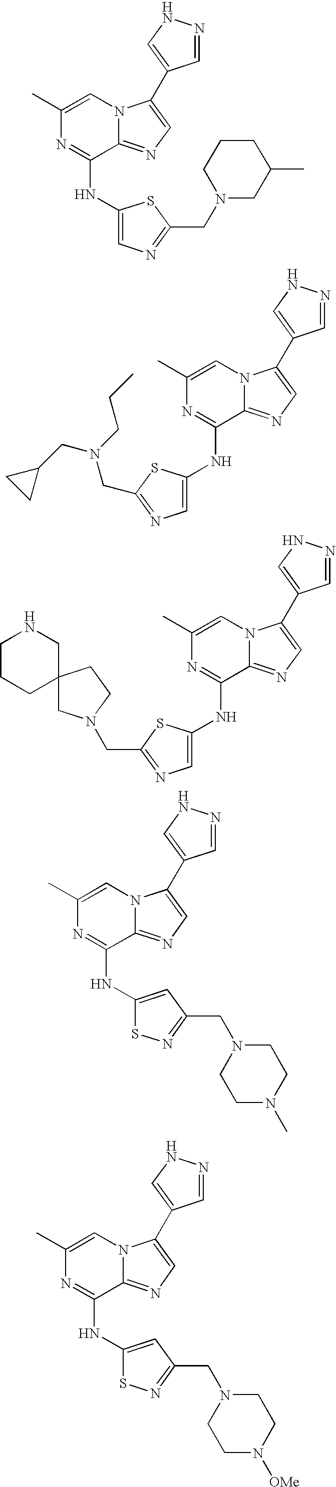 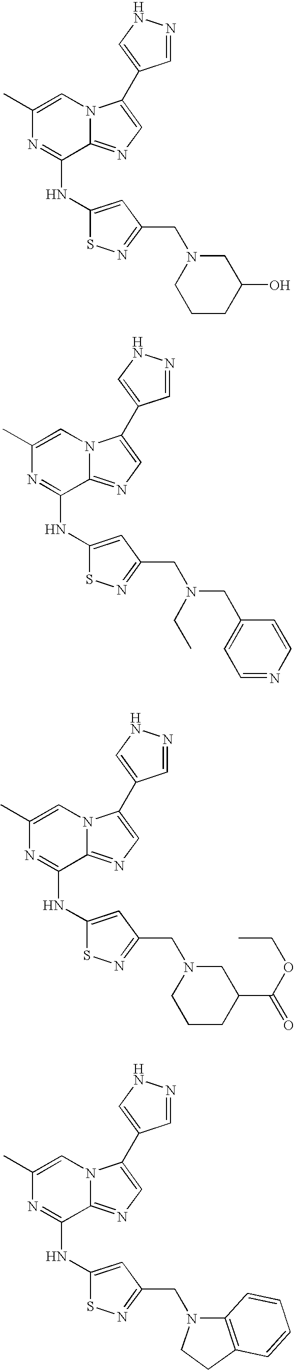 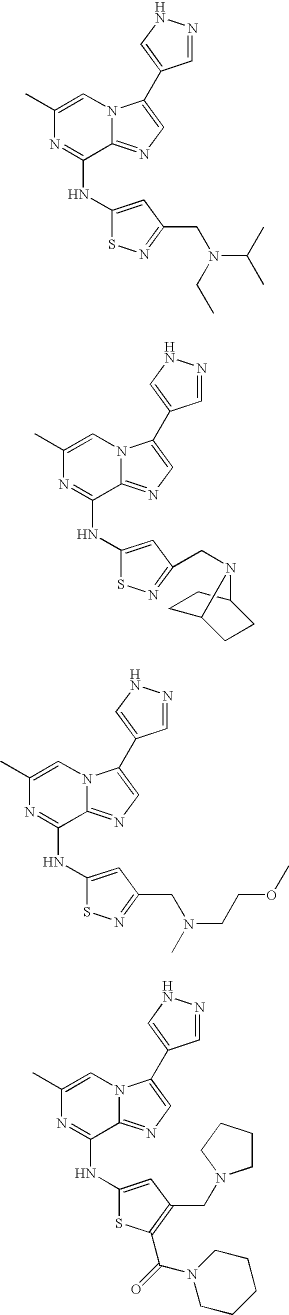 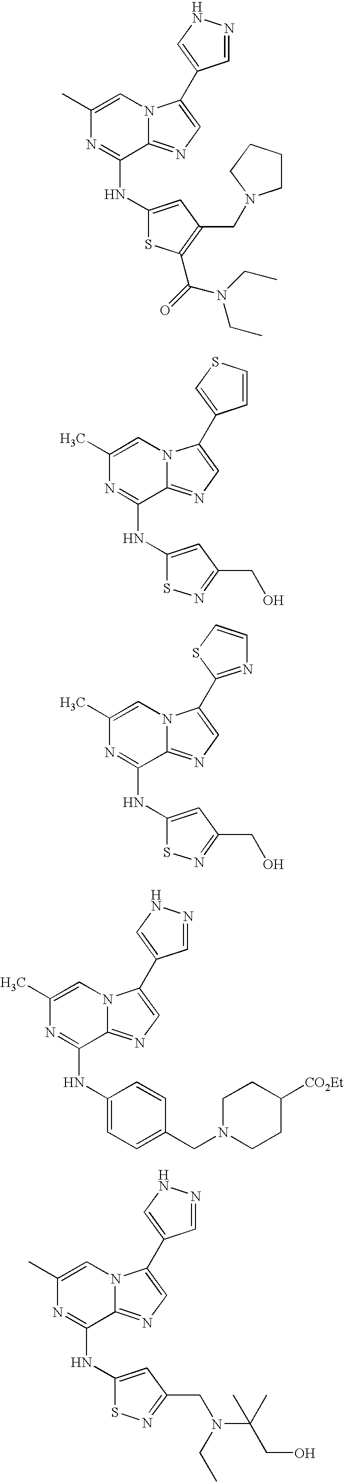 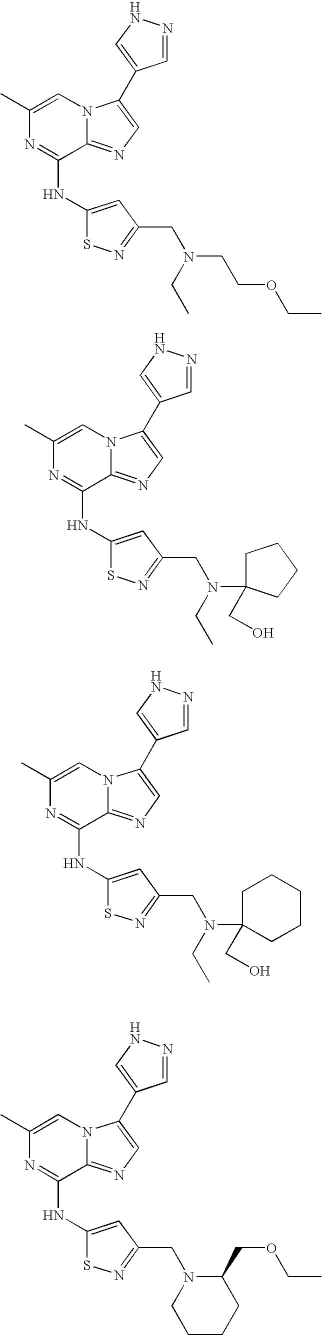 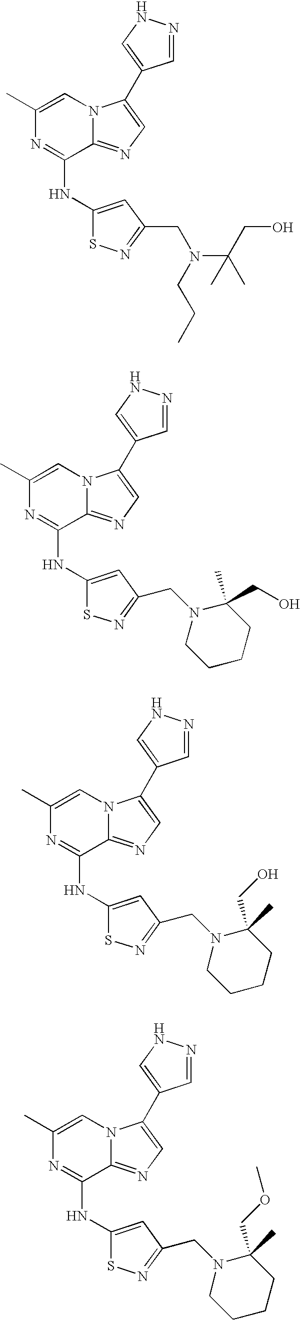 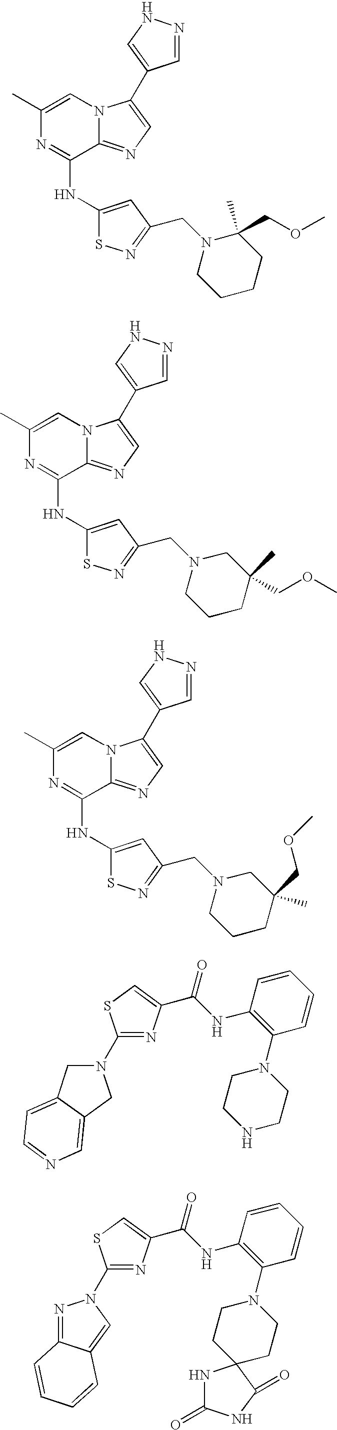 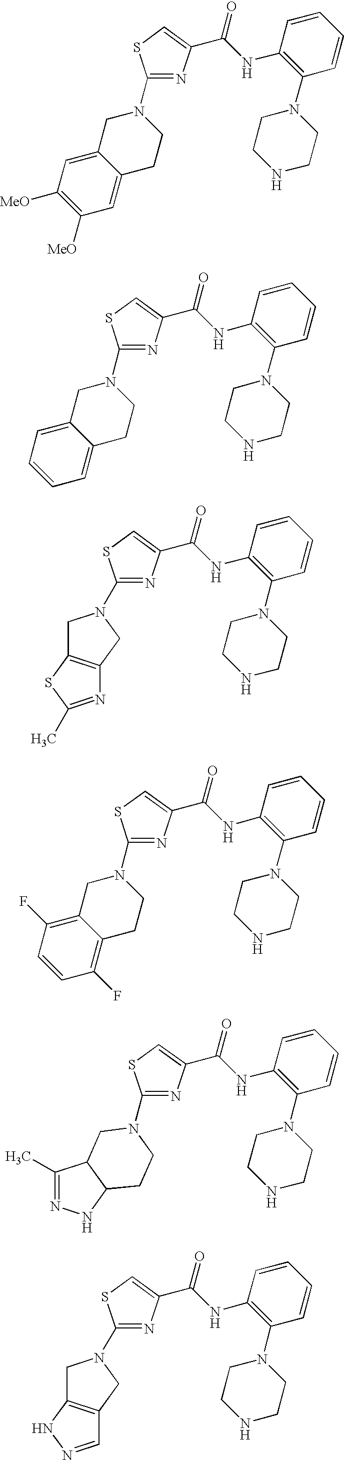 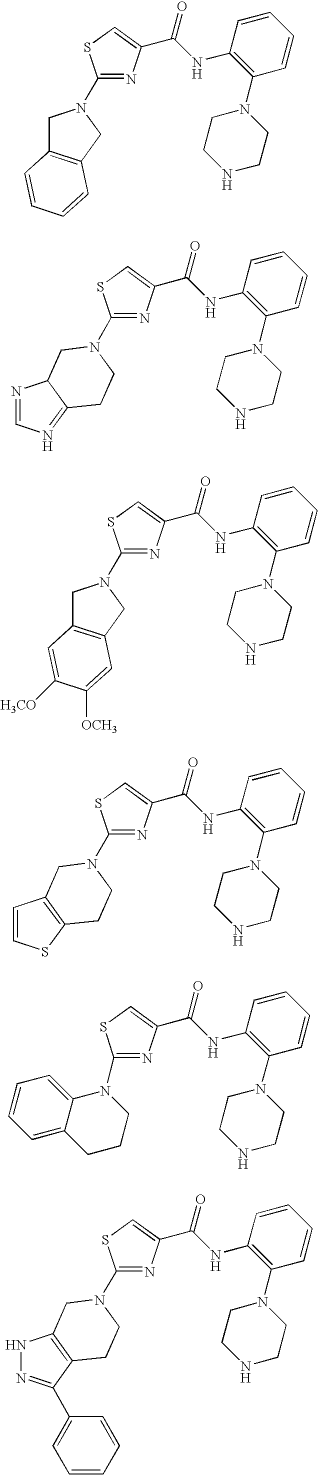 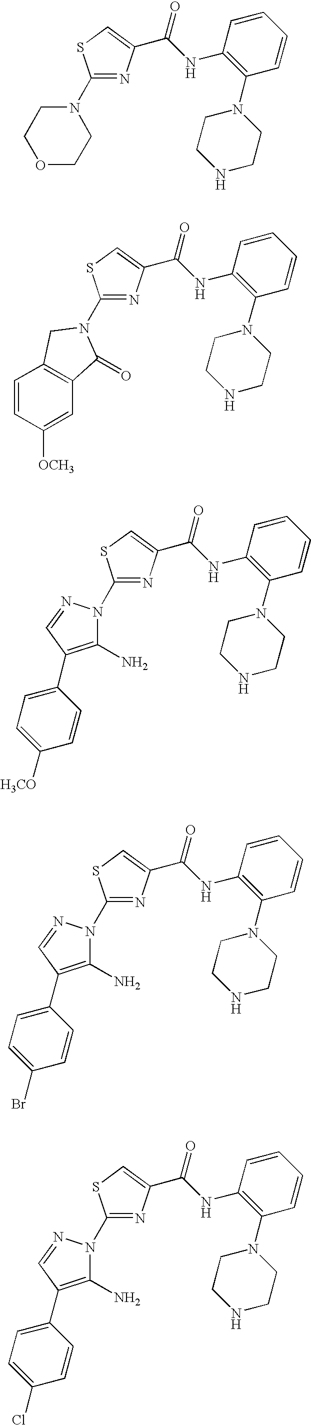 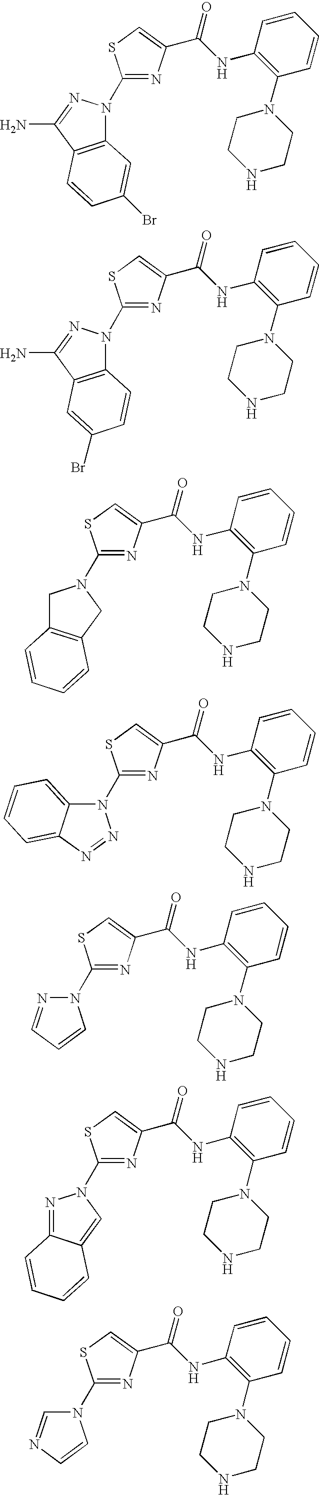 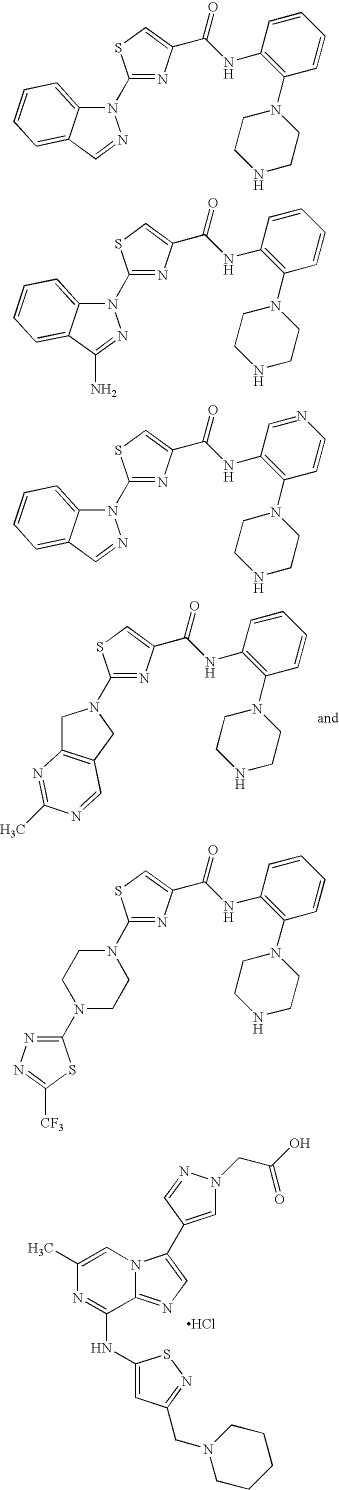 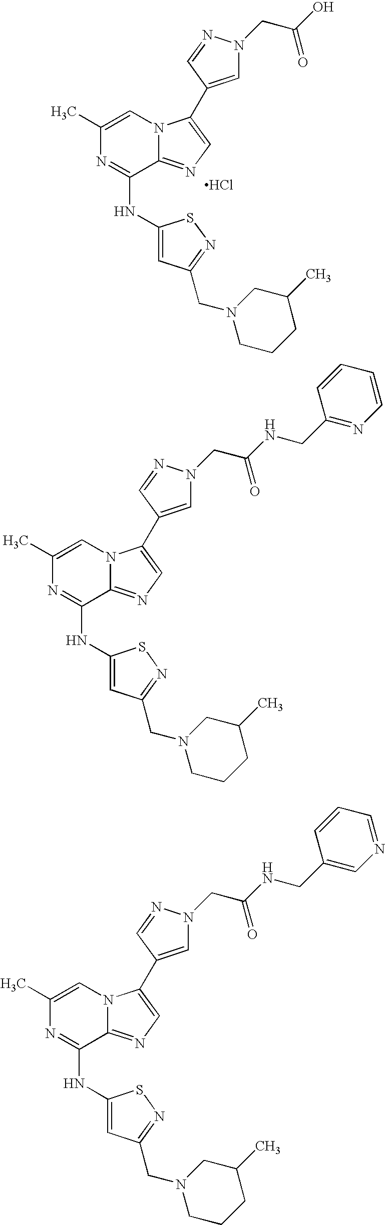 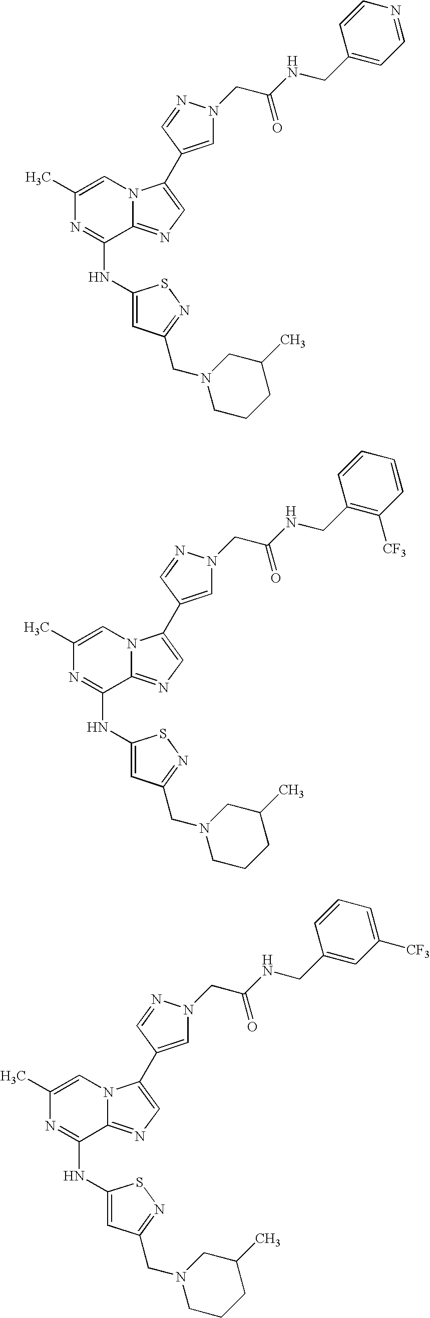 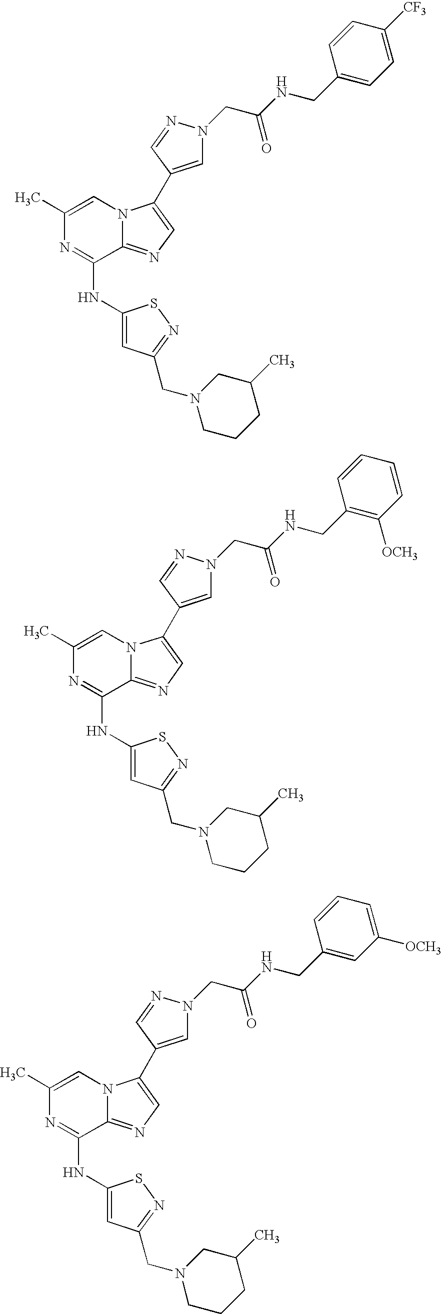 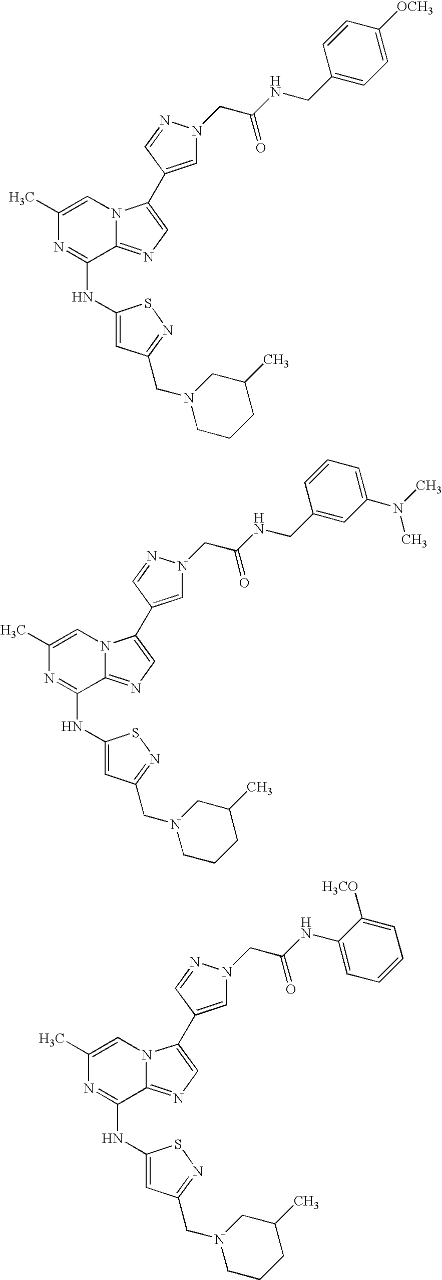 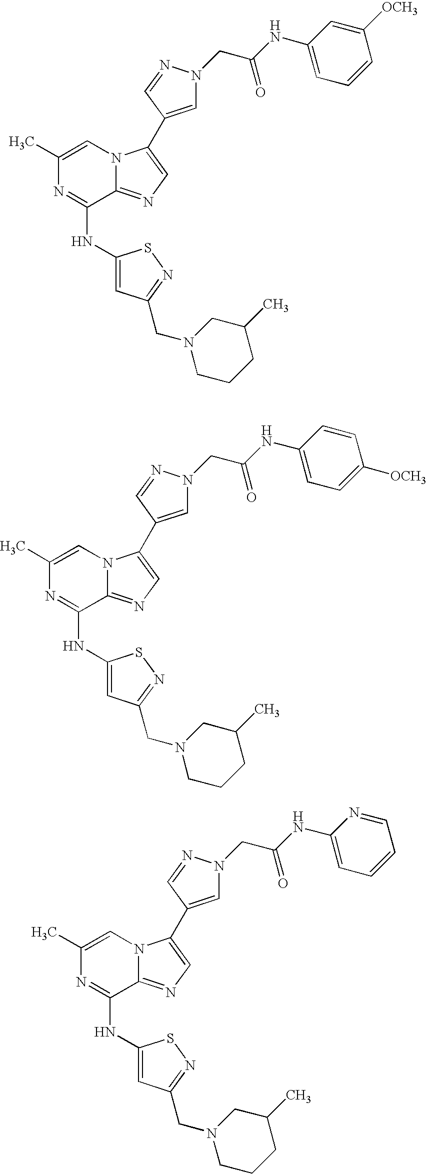 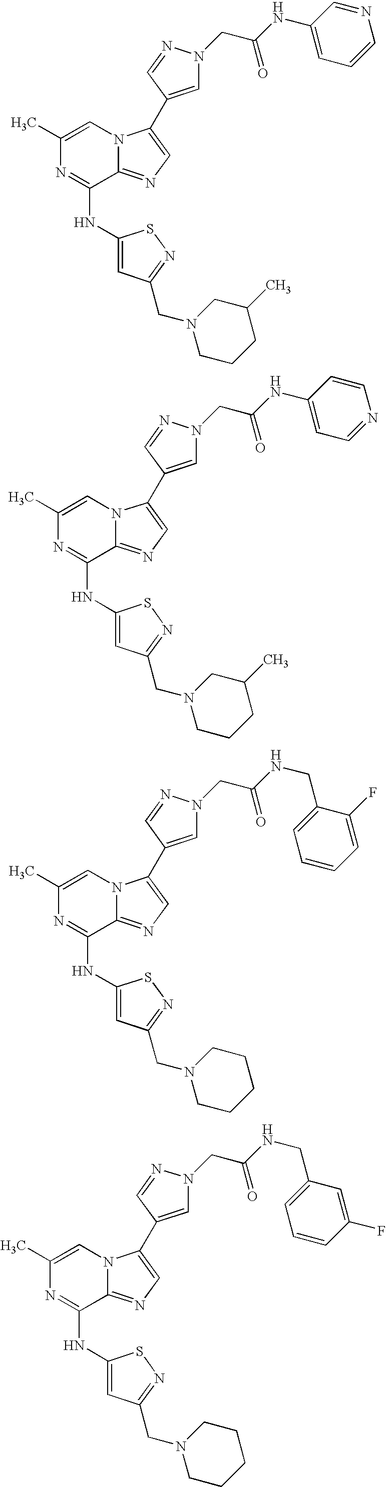 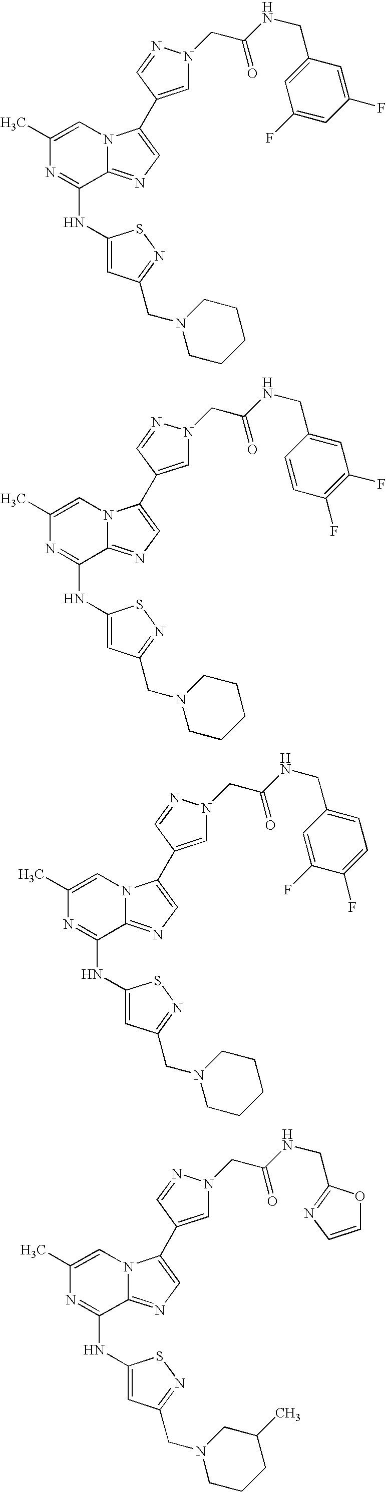 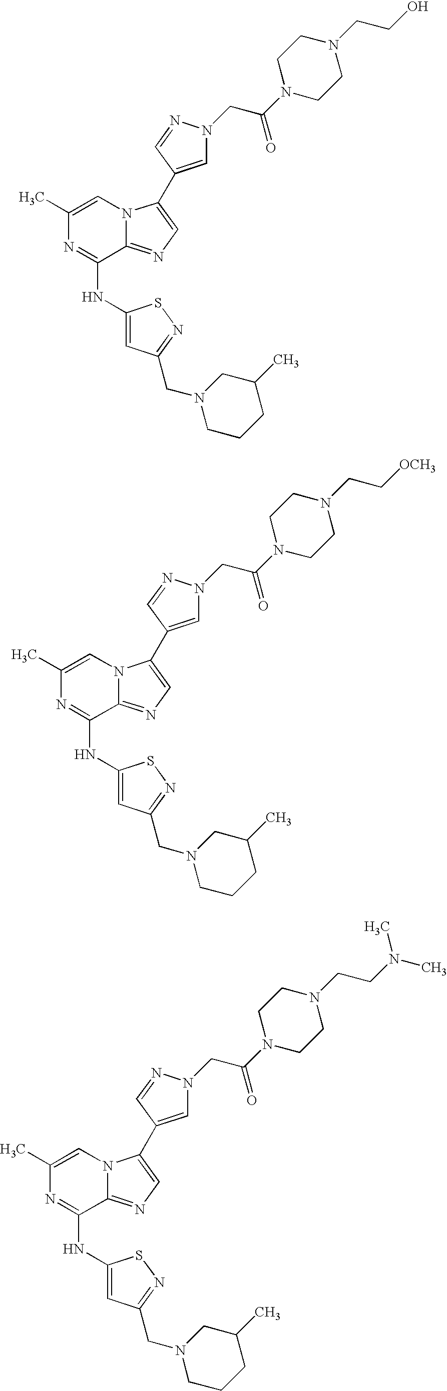 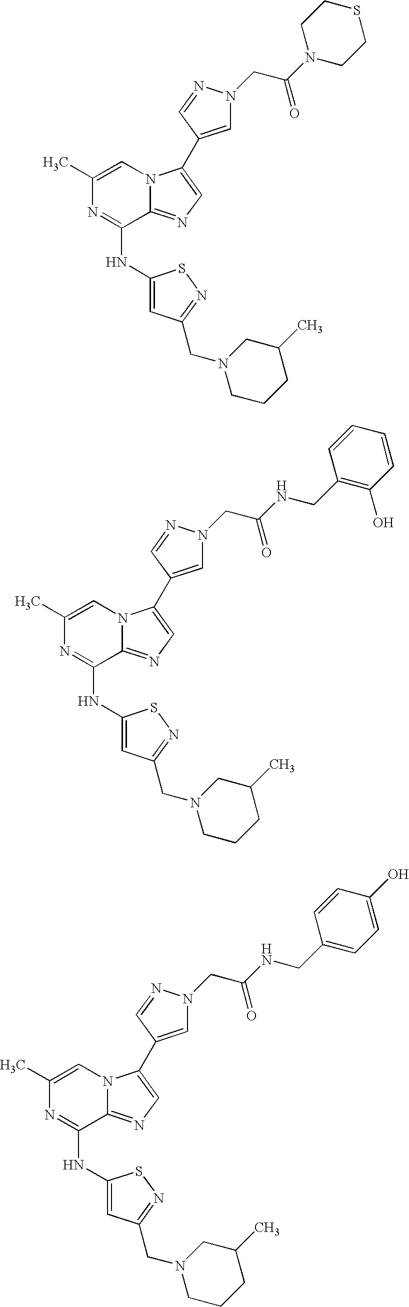 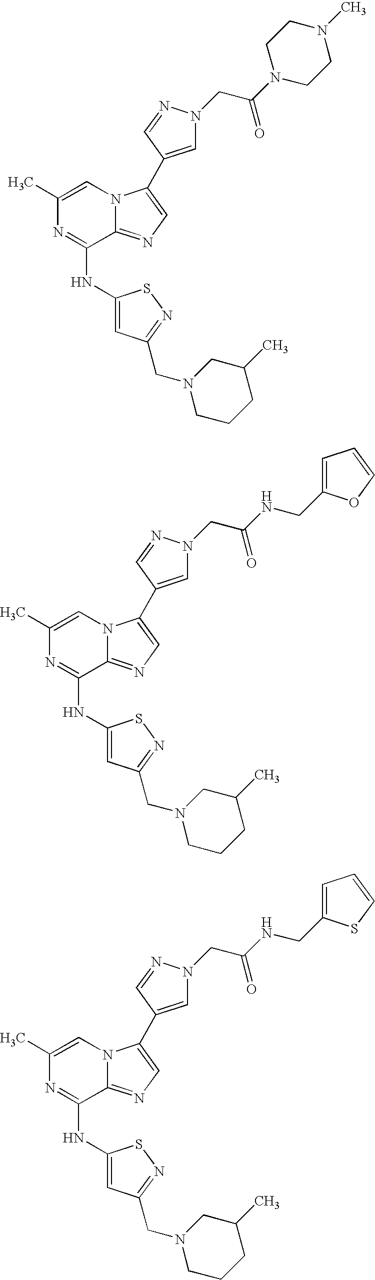 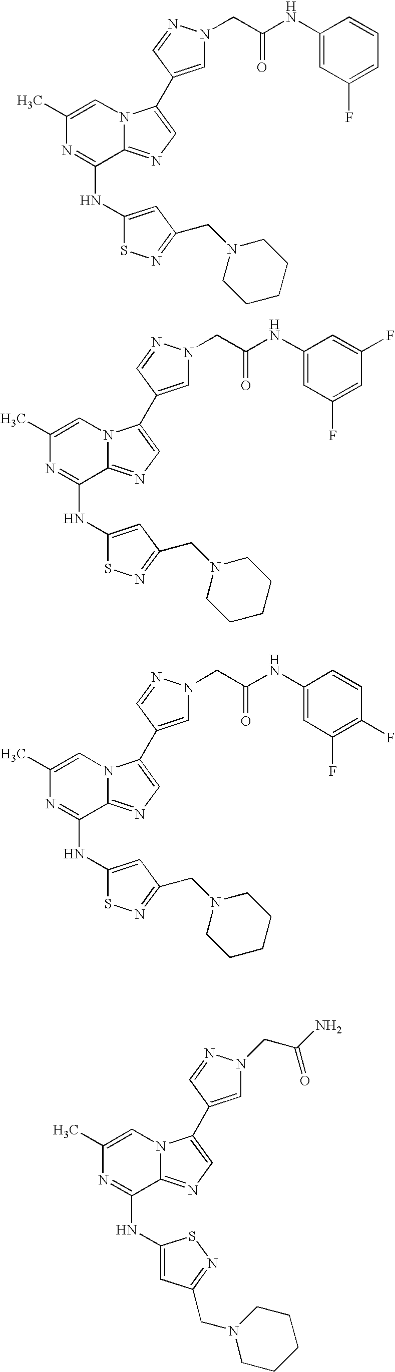 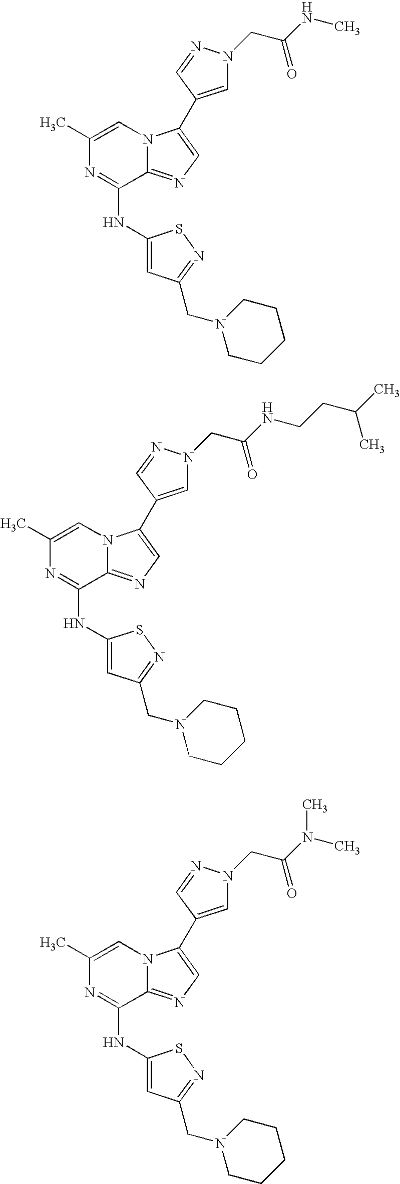 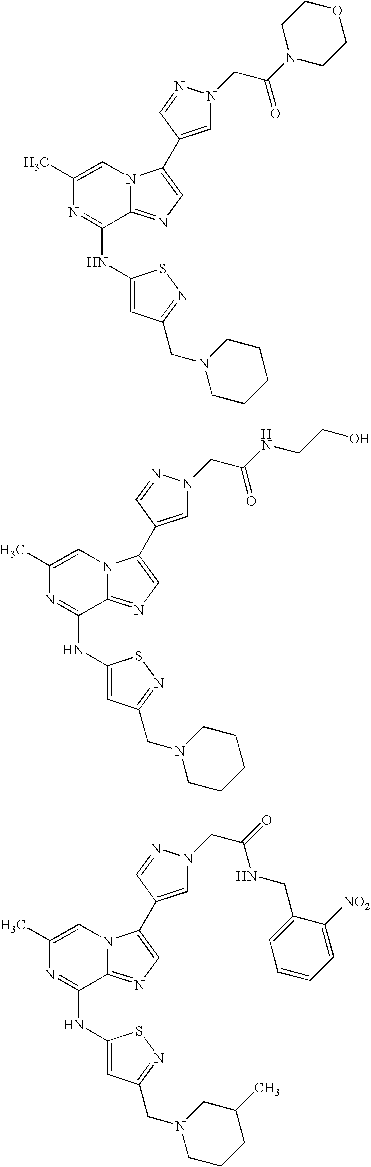 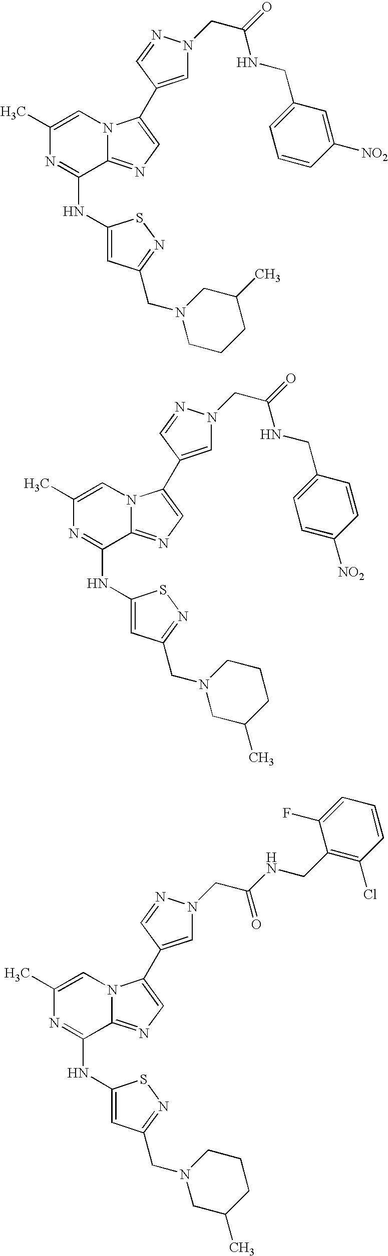 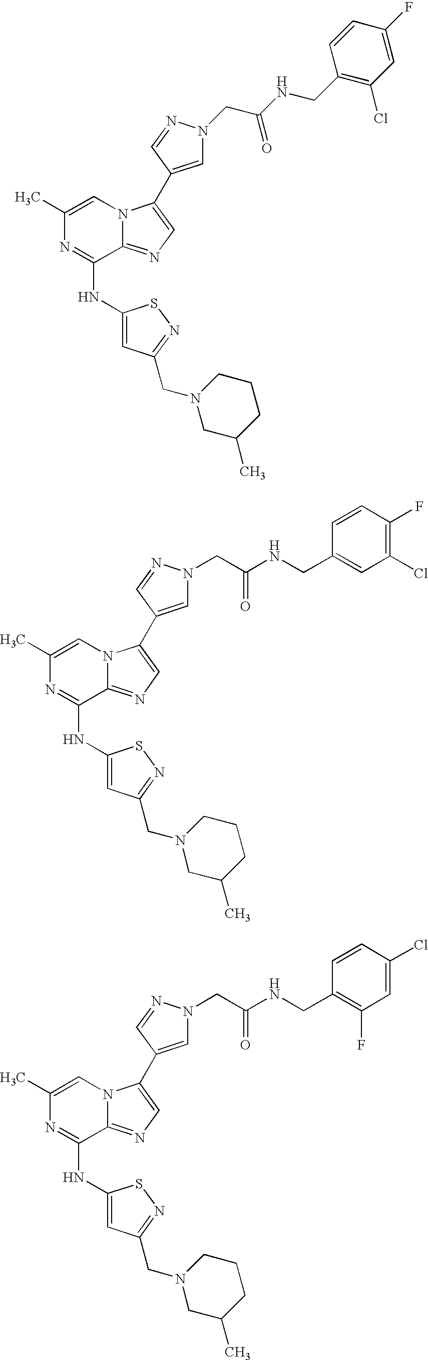 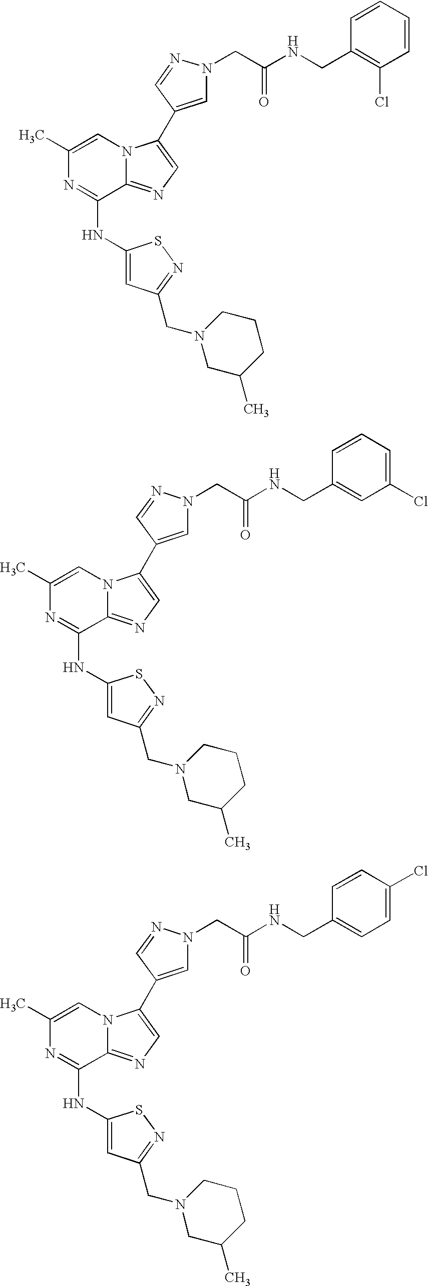 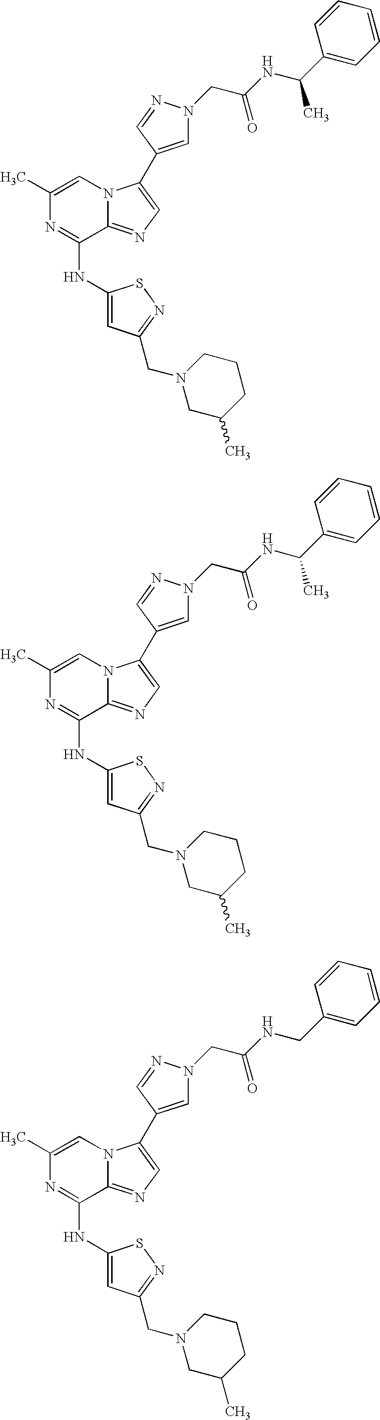 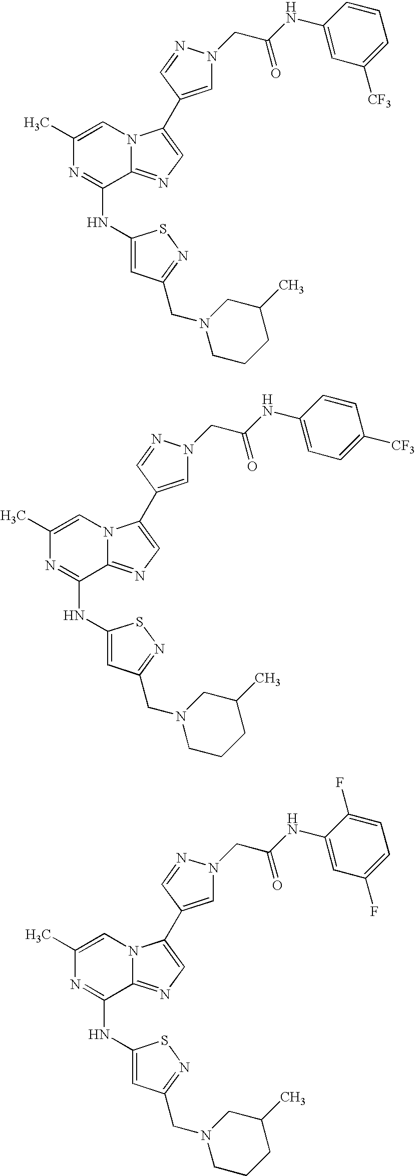 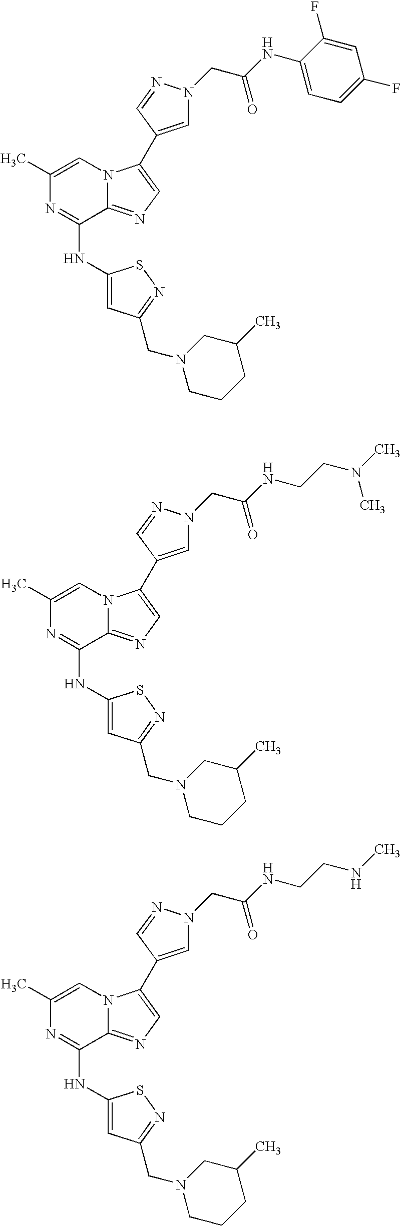 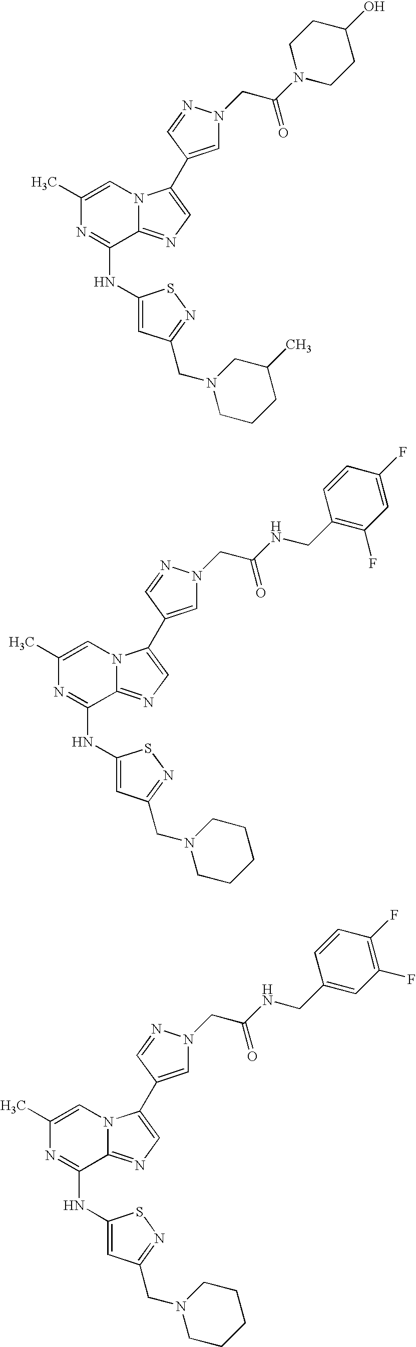 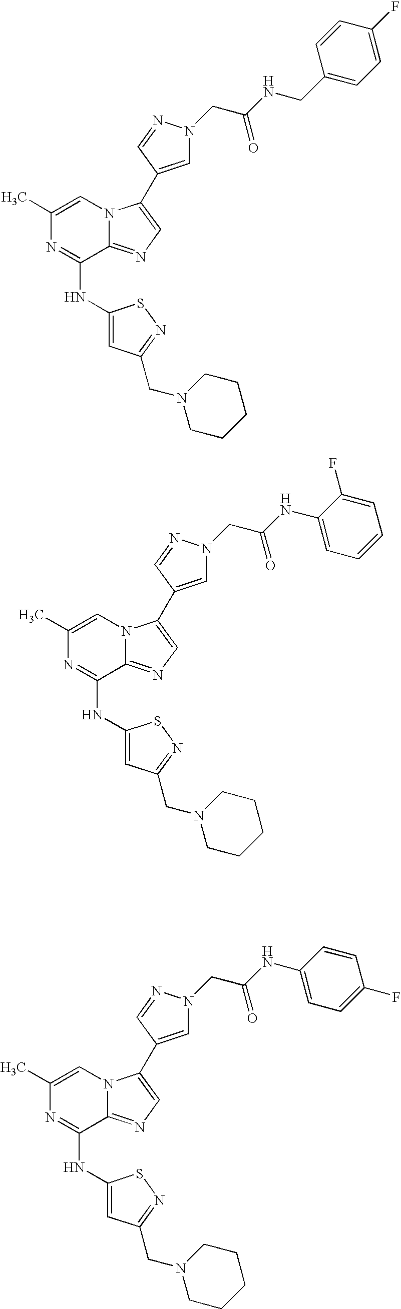 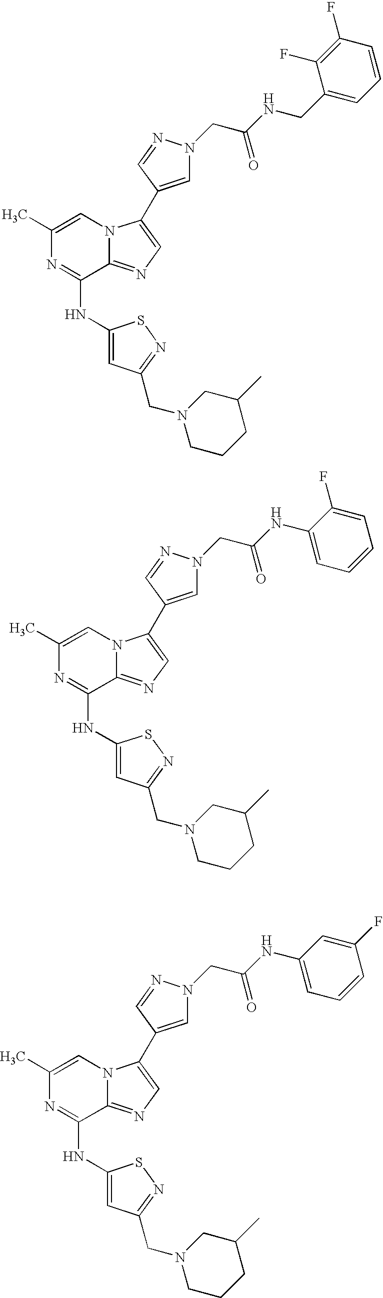 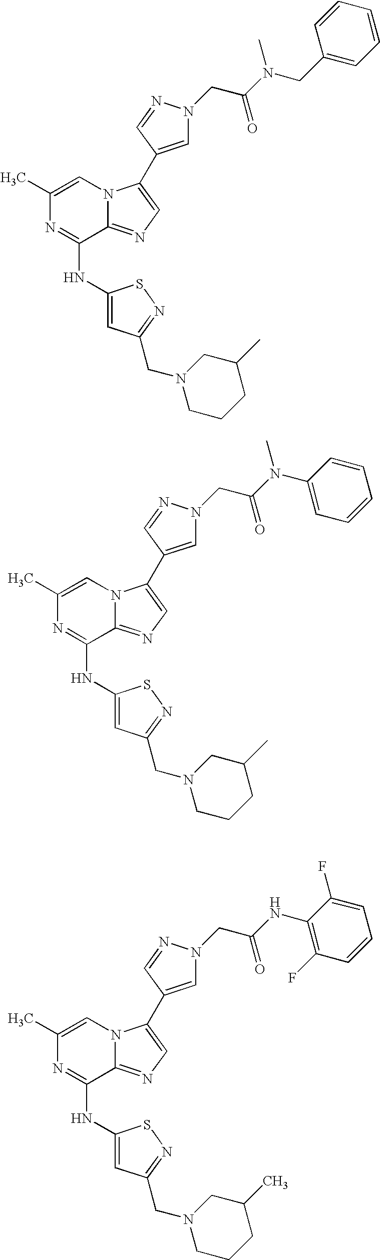 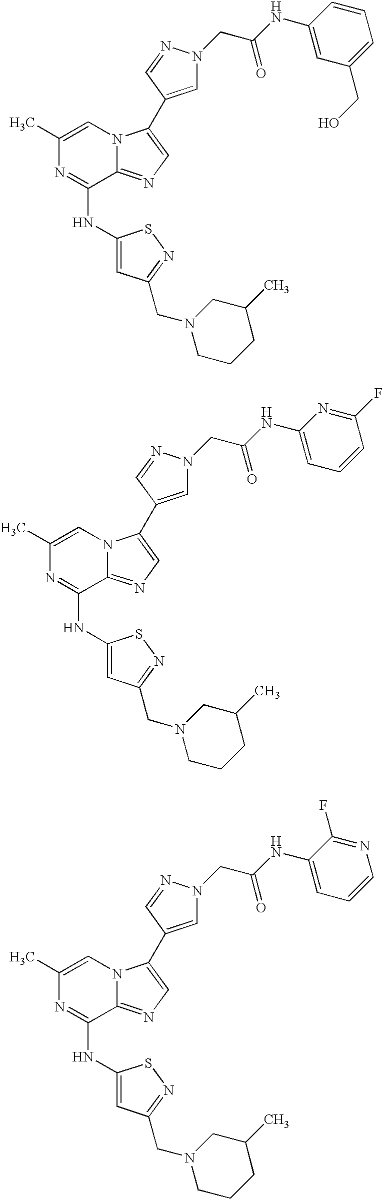 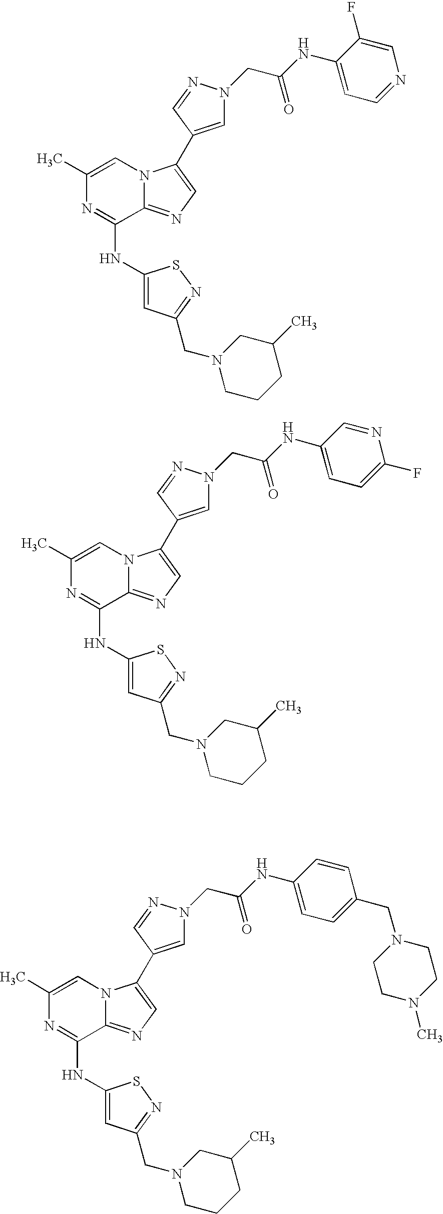 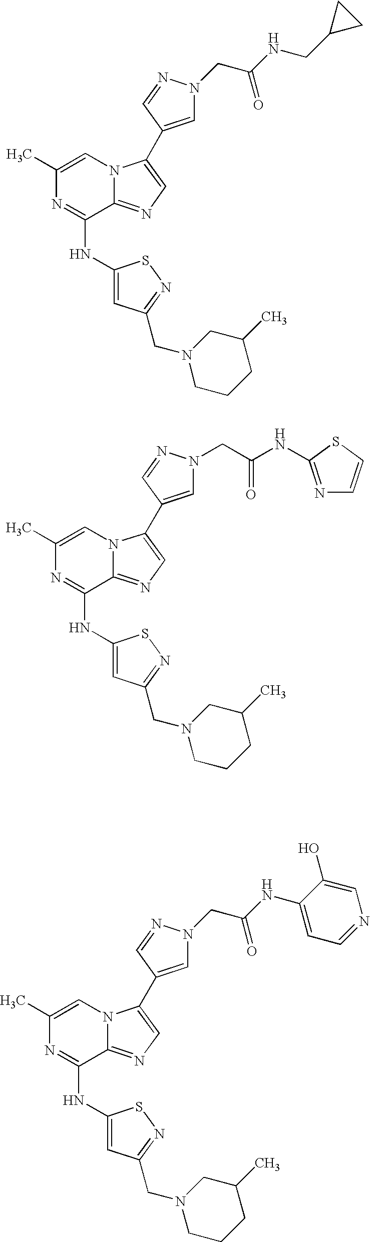 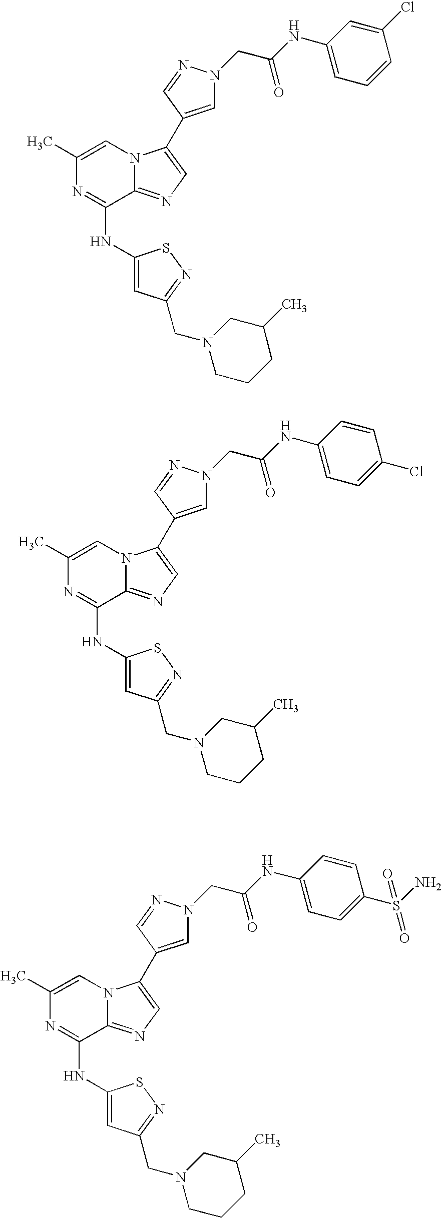 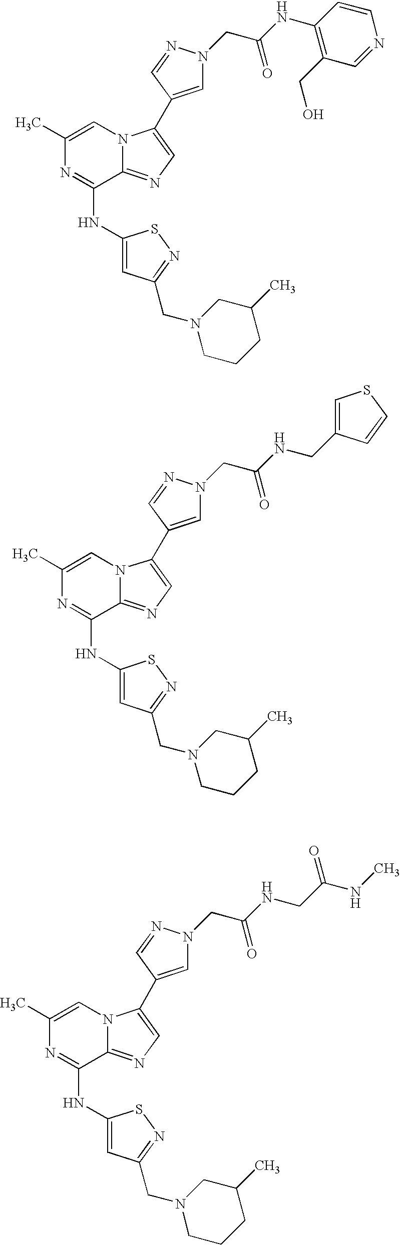 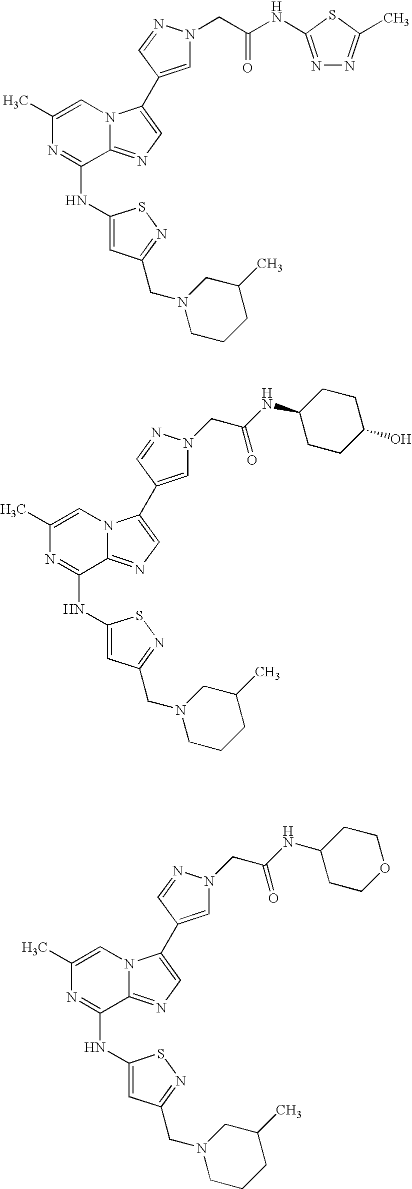 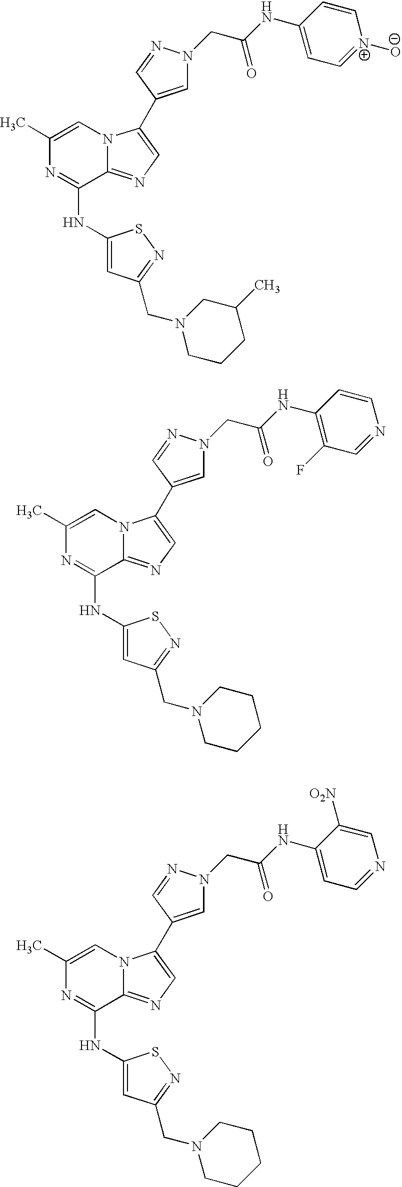 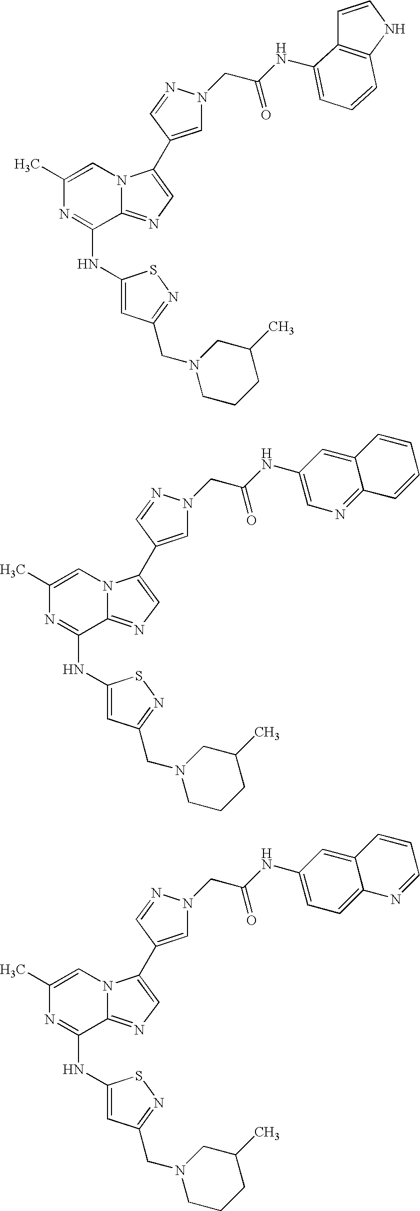 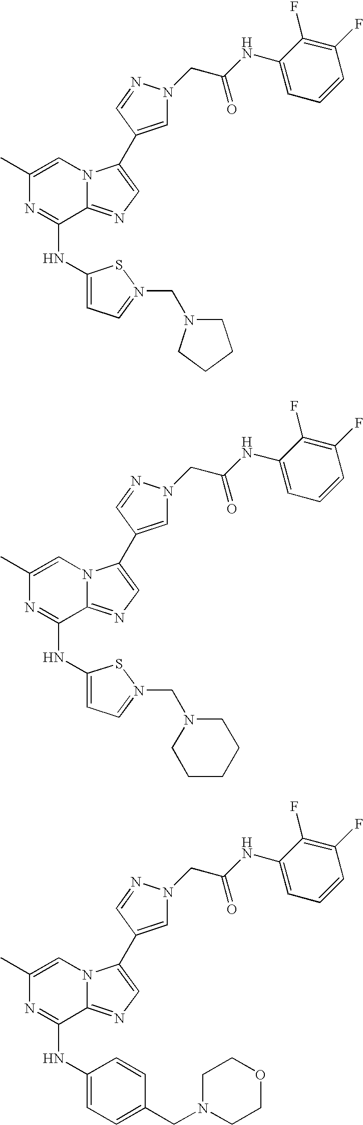 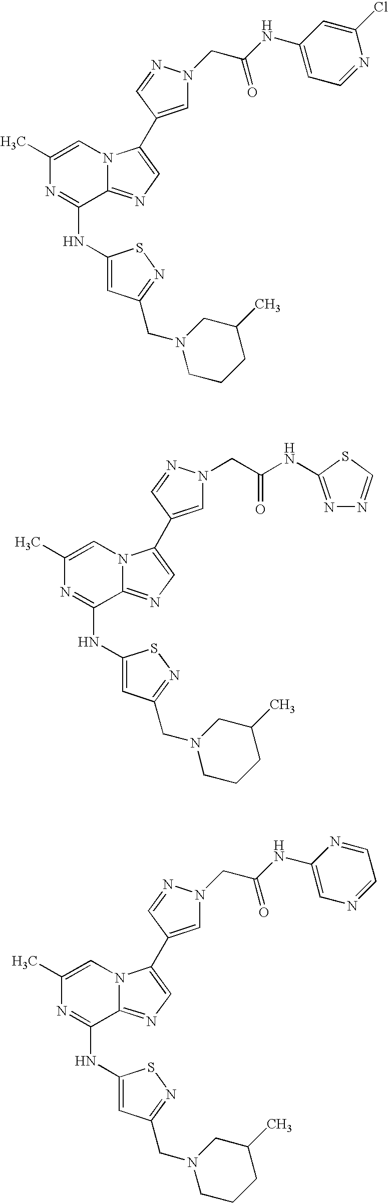 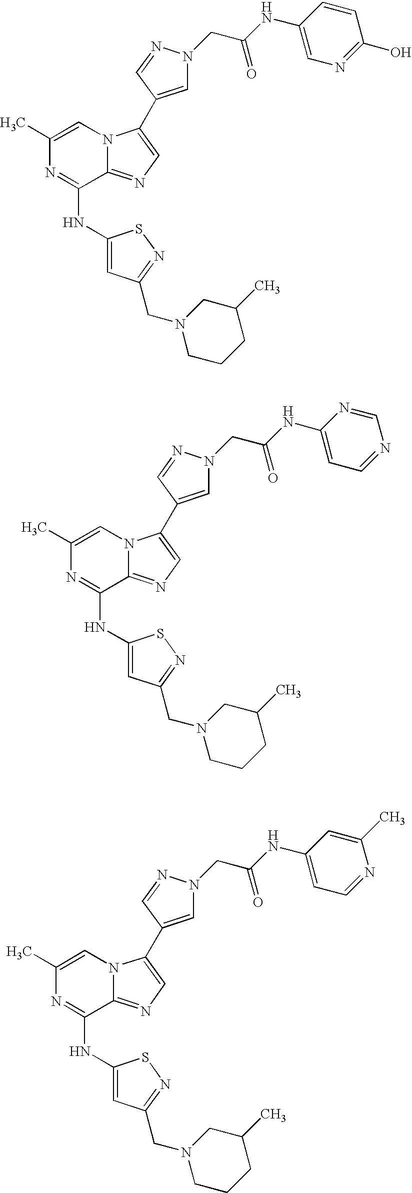 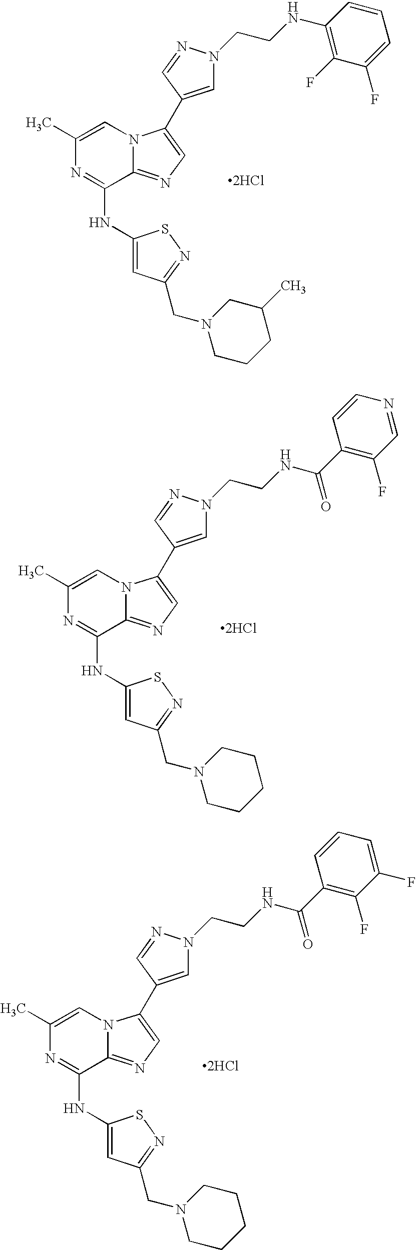 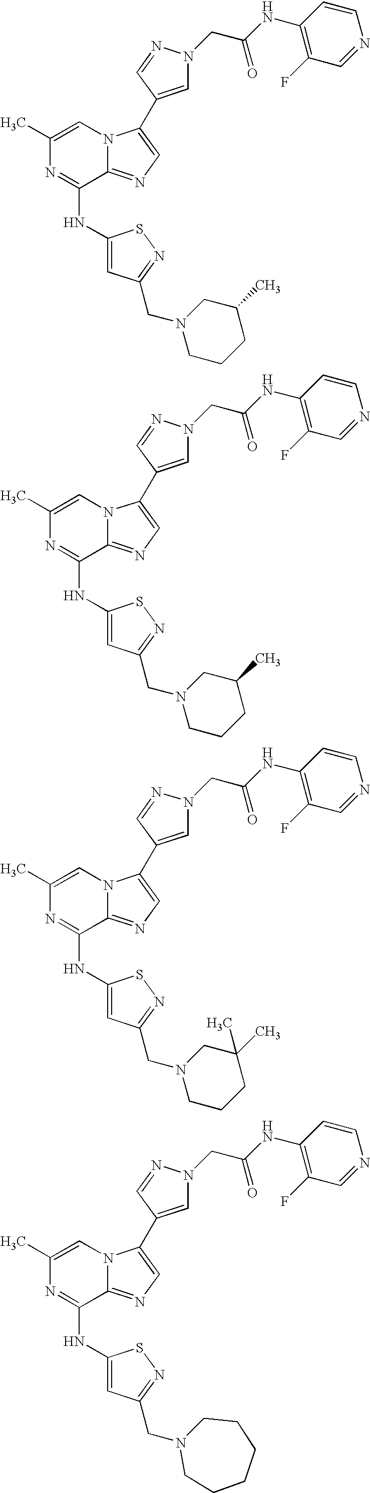 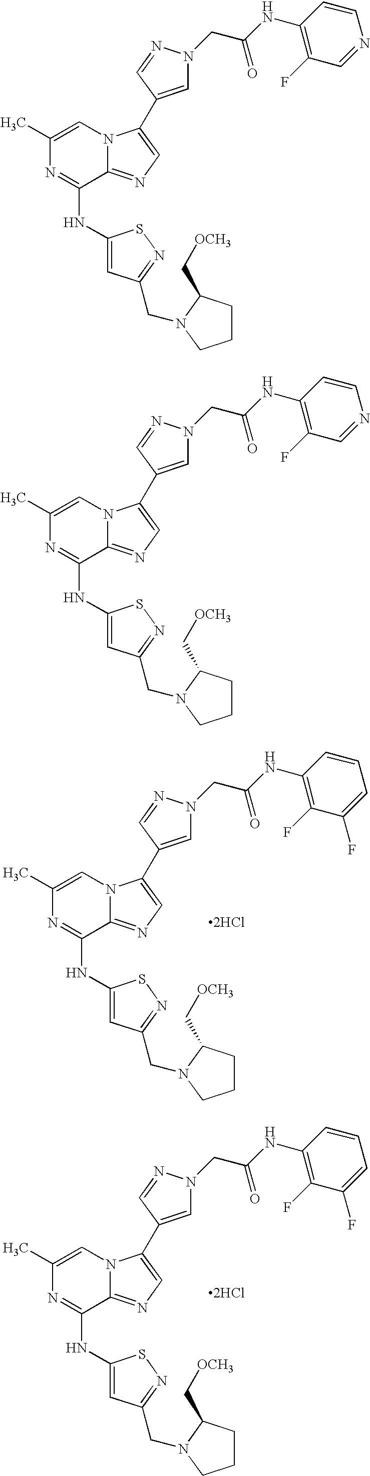 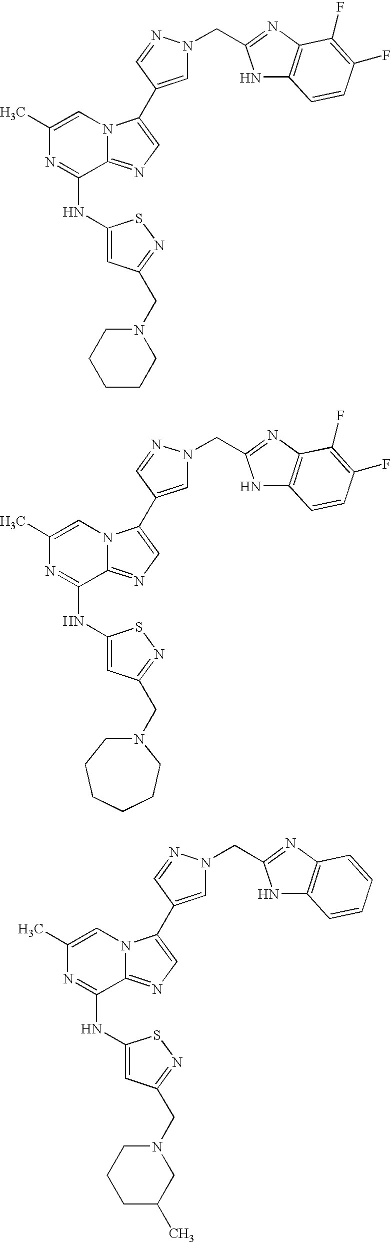 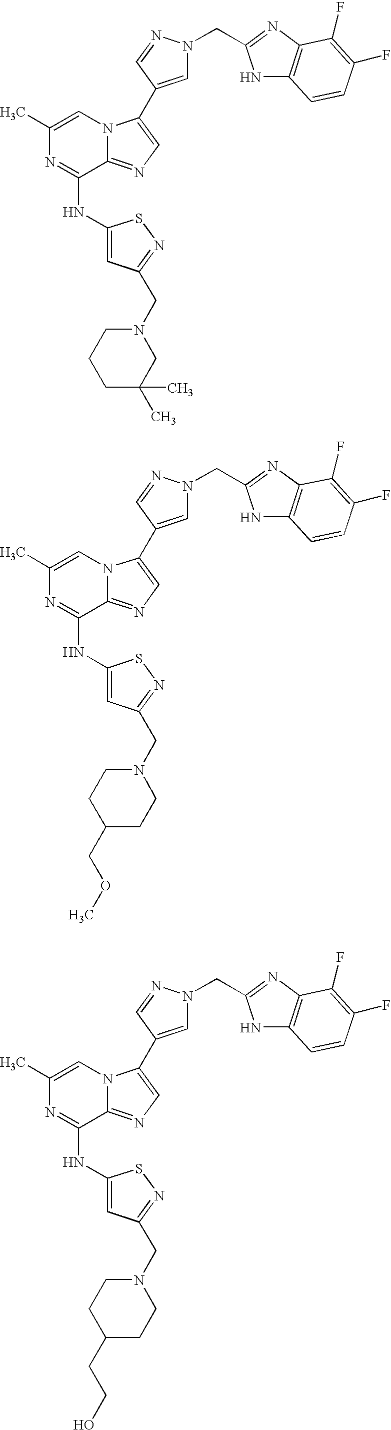 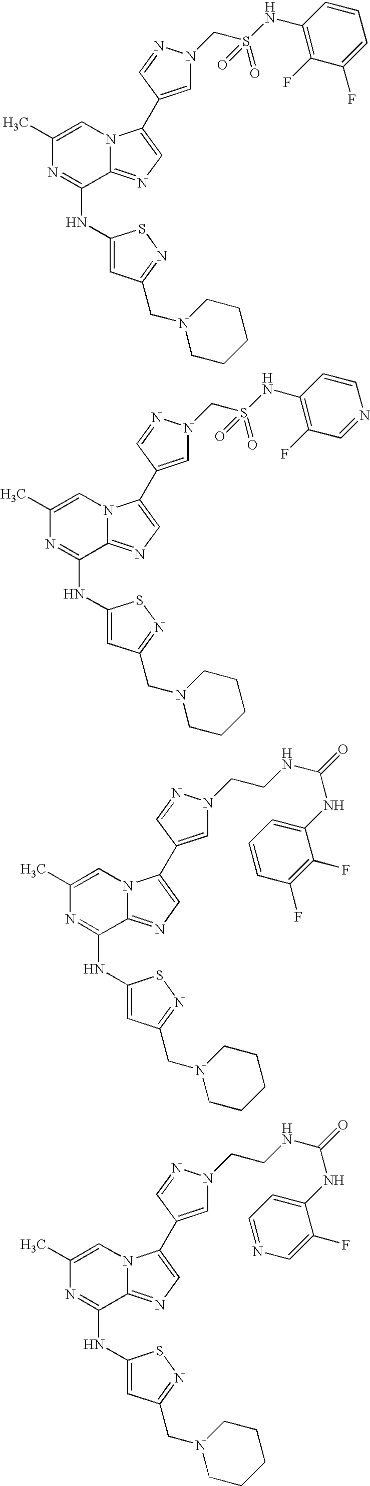 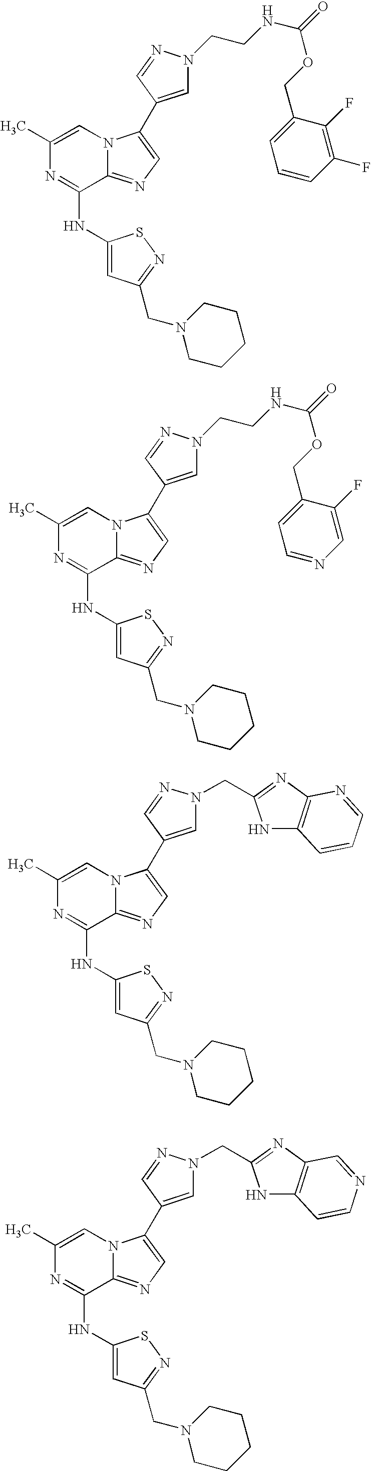 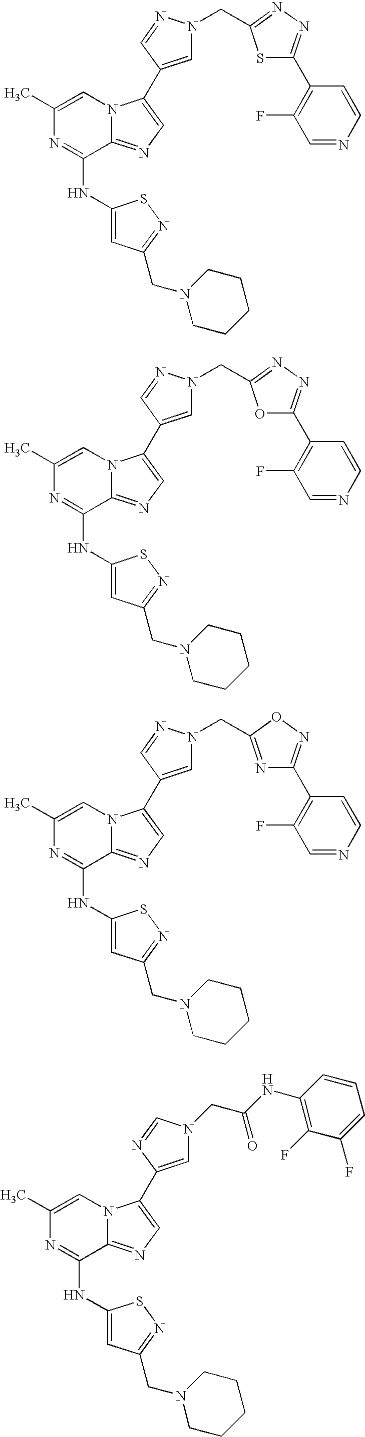 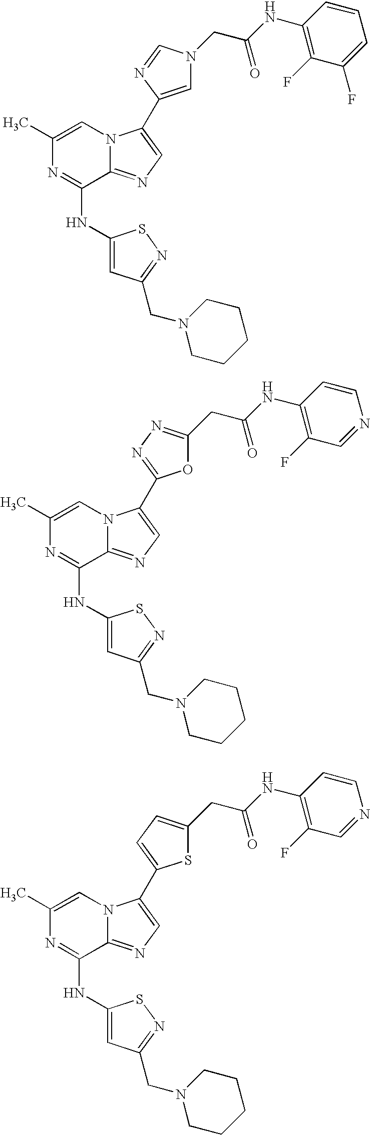 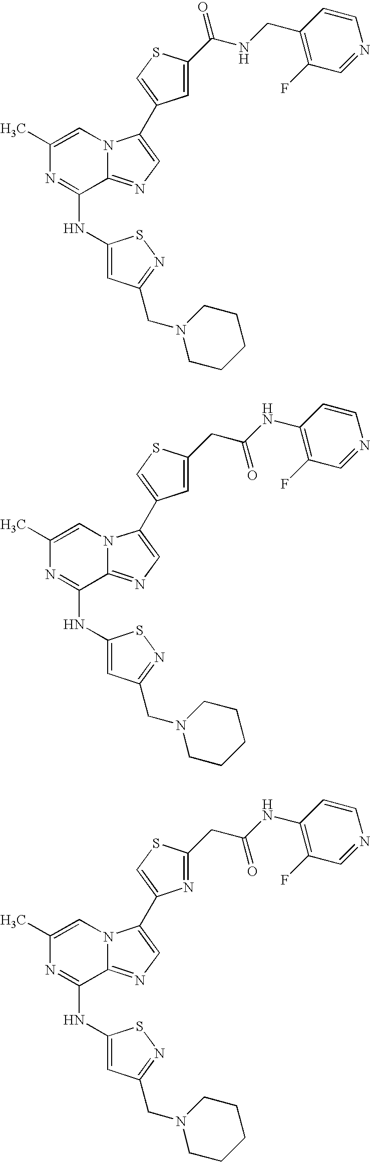 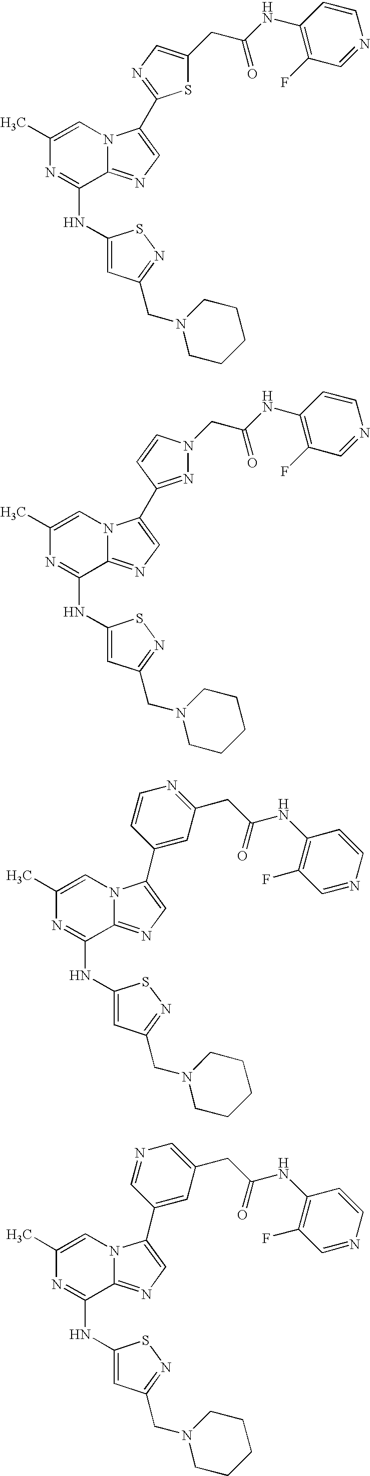 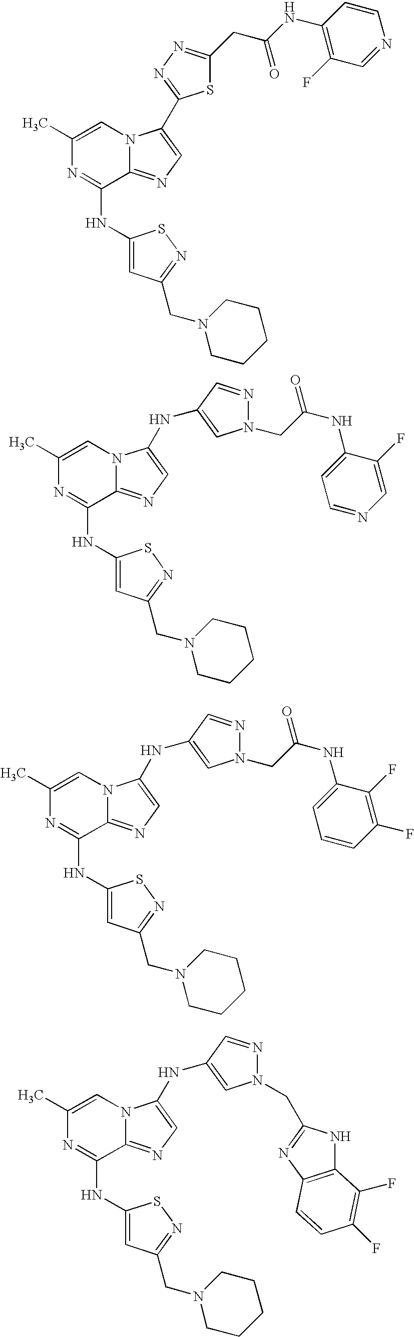 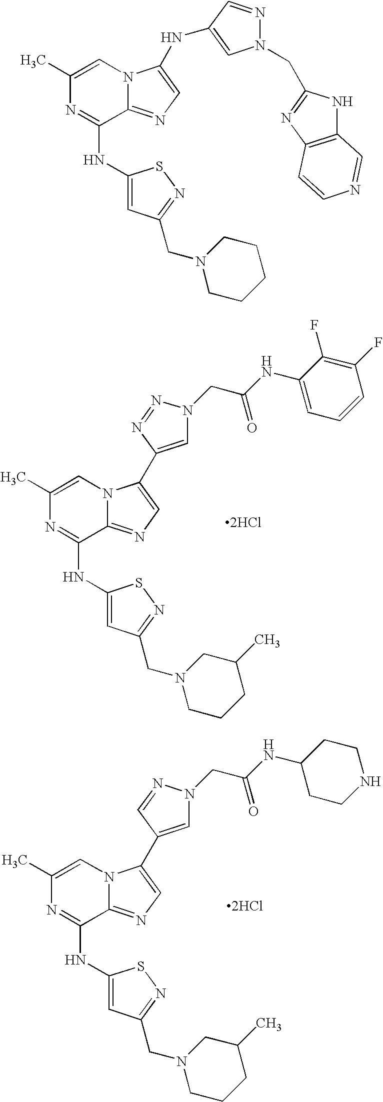 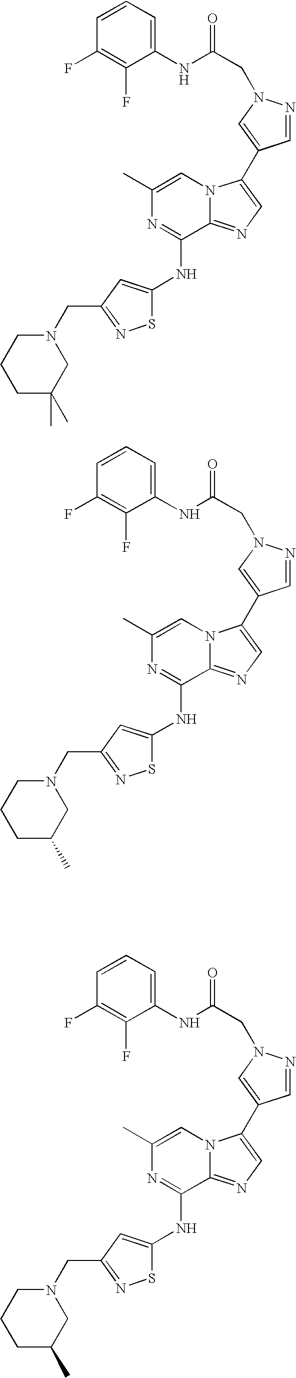
or a pharmaceutically acceptable salt, solvate, ester or prodrug thereof.

16. The pharmaceutical composition of claim 1, further comprising a time release agent, wherein said aurora kinase inhibitor is released after the release of said anti-mitotic agent.

17. The pharmaceutical composition of claim 14, further comprising a time release agent, wherein said aurora kinase inhibitor is released after the release of said anti-mitotic agent.

18. A method of treating or ameliorating cancer comprising administering the pharmaceutical composition of claim 1.

19. A method of treating or ameliorating cancer comprising administering the pharmaceutical composition of claim 17.
